SEQUENCE LISTING (856 sequences)

SEQ ID NO: 1 - skipped

SEQ ID NO: 2 - protein, 120 aa, synthetic construct
QVQLVQSGAEVKKPGESLKVSCKASGYTFTSYYMHWVRQAPGQGLEWMGIINPSGGSTSYAQKFQGRVTMTRDTSTSTVYMELSSLRSEDTAVYYCARGSAYYYDFADYWGQGTLVTVSS

SEQ ID NO: 3 - protein, 106 aa, synthetic construct
SYVLTQPSSVSVAPGQTATISCGGHNIGSKNVHWYQQRPGQSPVLVIYQDNKRPSGIPERFSGSNSGNTATLTISGTQAMDEADYYCQVWDNYSVLFGGGTKLTVL

SEQ ID NO: 4 - protein, 14 aa, synthetic construct
QCIHWDIETLLSCV

SEQ ID NO: 5 - protein, 14 aa, synthetic construct
VYCAEIGEYRVCRQ

SEQ ID NO: 6 - protein, 14 aa, synthetic construct
YMACSSGLSLCRLS

SEQ ID NO: 7 - protein, 18 aa, synthetic construct
GGVPWCTLDPGSLQCAWF

SEQ ID NO: 8 - protein, 20 aa, synthetic construct
QCVHWDLDTLFGCIREQLEL

SEQ ID NO: 9 - protein, 22 aa, synthetic construct
VHRIPWCTLDPGGLQCAWLRQM

SEQ ID NO: 10 - protein, 23 aa, synthetic construct
GGRSCLWQPGALHCTWWAEEEPV

SEQ ID NO: 11 - protein, 22 aa, synthetic construct
GWGIPWCTLDPGSLQCAWLGKH

SEQ ID NO: 12 - protein, 21 aa, synthetic construct
GNYTCEVTELTREGETIIELK

SEQ ID NO: 13 - protein, 32 aa, synthetic construct
SETTTGTSSNSSQSTSNSGLAPNPTNATTKAA

SEQ ID NO: 14 - protein, 5 aa, synthetic construct
LPXTG

SEQ ID NO: 15 - protein, 135 aa, Homo sapiens [sequence omitted]

SEQ ID NO: 16 - protein, 448 aa, synthetic construct
QVQLVESGGGVVQPGRSLRLSCAASGFTFSSYTMHWVRQAPGKGLEWVTFISYDGNNKYYADSVKGRFTISRDNSKNTLYLQMNSLRAEDTAIYYCARTGWLGPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 17 - protein, 215 aa, synthetic construct
EIVLTQSPGTLSLSPGERATLSCRASQSVGSSYLAWYQQKPGQAPRLLIYGAFSRATGIPDRFSGSGSGTDFTLTISRLEPEDFAVYYCQQYGSSPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 18 - protein, 91 aa, Homo sapiens
MKVSAAALAVILIATALCAPASASPYSSDTTPCCFAYIARPLPRAHIKEYFYTSGKCSNPAVVFVTRKNRQVCANPEKKWVREYINSLEMS

SEQ ID NO: 19 - protein, 178 aa, synthetic construct
MTVLAPAWSPTTYLLLLLLLSSGLSGTQDCSFQHSPISSDFAVKIRELSDYLLQDYPVTVASNLQDEELCGGLWRLVLAQRWMERLKTVAGSKMQGLLERVNTEIHFVTKCAFQPPPSCLRFVQTNISRLLQETSEQLVALKPWITRQNFSRCLELQCQPVETVFHRVSQDGLDLLTS

SEQ ID NO: 20 - protein, 219 aa, synthetic construct
MCPARSLLLVATLVLLDHLSLARNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 21 - protein, 328 aa, synthetic construct
MCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 22 - protein, 177 aa, Homo sapiens
MFHVSFRYIFGLPPLILVLLPVASSDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEH

SEQ ID NO: 23 - protein, 153 aa, Homo sapiens
MYRMQLLSCIALSLALVTNSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 24 - protein, 162 aa, Homo sapiens
MRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 25-29 - skipped (5 entries)

SEQ ID NO: 30 - protein, 441 aa, synthetic construct
EVQLLESGGGLVQPGGSLRLSCAASGFTFSSYPMSWVRQAPGKGLEWVSAIGGSGGSLPYADIVKGRFTISRDNSKNTLYLQMNSLRAEDTAVYYCARYWPMDIWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCEVECPECPAPPVAGPSVFLFPPKPKDTLMISRTPEVTCVVVAVSHEDPEVQFNWYVDGVEVHNAKTKPREEQFNSTFRVVSVLTVVHQDWLNGKEYKCKVSNKGLPSSIEKTISKTKGQPREPQVYTLPPSREEMTKNQVSLTCEVKGFYPSDIAVEWESNGQPENNYKTTPPMLDSDGSFFLYSKLTVDKSRWQQGNVFSCSVMHEALHNHYTQKSLSLSPGK

SEQ ID NO: 31 - protein, 215 aa, synthetic construct
EIVLTQSPGTLSLSPGERATLSCRASQSVSSSYLAWYQQKPGQAPRLLMYDASIRATGIPDRFSGSGSGTDFTLTISRLEPEDFAVYYCQQYQSWPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 32 - protein, 446 aa, synthetic construct
EVQLLESGGGLVQPGGSLRLSCAASGFTFSDNAMGWVRQAPGKGLEWVSAISGPGSSTYYADSVKGRFTISRDNSKNTLYLQMNSLRAEDTAVYYCAKVLGWFDYWGQGTLVTVSSASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDEKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 33 - protein, 216 aa, synthetic construct
EIVLTQSPGTLSLSPGERATLSCRASQSVSDEYLSWYQQKPGQAPRLLIHSASTRATGIPDRFSGSGSGTDFTLAISRLEPEDFAVYYCQQYGYPPDFTFGQGTKVEIKRTVAAPSVFIFPPSDRKLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 34 - protein, 446 aa, synthetic construct; identical to SEQ ID NO: 32

SEQ ID NO: 35 - protein, 451 aa, synthetic construct
EVQLVESGGGLVQPGGSLRLSCAASGFTFSNFWMTWVRQAPGKGLEWVANMNQDGSEKYYVDSVKGRFTISRDNAKSSLYLQMNSLRAEDTAVYYCARDREYCISTSCYDDFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 36 - protein, 448 aa, synthetic construct
QLQLQESGPGLVKPSETLSLTCTVSGGSISSGSYFWGWIRQPPGKGLEWIGSIYYSGITYYNPSLKSRVTISVDTSKNQFSLKLSSVTAADTAVYYCARHDGAVAGLFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 37 - protein, 214 aa, synthetic construct
SYVLTQPPSVSVAPGQTARITCGGNNIGSKSVHWYQQPPGQAPVVVVYDDSDRPSGIPERFSGSNSGNTATLTISRVEAGDEAVYYCQVWDSSSDHVVFGGGTKLTVLGQPKAAPSVTLFPPSSEELQANKATLVCLISDFYPGAVTVAWKGDSSPVKAGVETTTPSKQSNNKYAASSYLSLTPEQWKSHRSYSCQVTHEGSTVEKTVAPTECS

SEQ ID NO: 38 - protein, 451 aa, synthetic construct
QVQLVQSGAEVKKPGSSVKVSCKASGGTFSNYWMHWVRQAPGQGLEWMGATYRGHSDTYYNQKFKGRVTITADKSTSTAYMELSSLRSEDTAVYYCARGAIYDGYDVLDNWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKENWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 39 - protein, 214 aa, synthetic construct
DIQMTQSPSSLSASVGDRVTITCSASQDISNYLNWYQQKPGKAPKLLIYYTSNLHSGVPSRFSGSGSGTDFTLTISSLQPEDFATYYCQQYRKLPWTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 40 - protein, 986 aa, synthetic construct
QVQLVQSGAEVKKPGASVKVSCKASGYTFTNHIIHWVRQAPGQCLEWMGYINPYPGYHAYNEKFQGRATMTSDTSTSTVYMELSSLRSEDTAVYYCARDGYYRDTDVLDYWGQGTLVTVSSGGGGSGGGGSGGGGSDIQMTQSPSSLSASVGDRVTITCQASQDISNYLNWYQQKPGKAPKLLIYYTSRLHTGVPSRFSGSGSGTDFTFTISSLEPEDIATYYCQQGNTLPWTFGCGTKVEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 41 - protein, 500 aa, synthetic construct
QVQLVQSGAEVKKPGASVKVSCKASGYTFTNHIIHWVRQAPGQGLEWMGYINPYPGYHAYNEKFQGRATMTSDTSTSTVYMELSSLRSEDTAVYYCARDGYYRDTDVLDYWGQGTLVTVSSGGGGSGGGGSGGGGSDIQMTQSPSSLSASVGDRVTITCQASQDISNYLNWYQQKPGKAPKLLIYYTSRLHTGVPSRFSGSGSGTDFTFTISSLEPEDIATYYCQQGNTLPWTFGQGTKVEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLHH

SEQ ID NO: 42 - protein, 121 aa, synthetic construct
QVQLVQSGAEVKKPGASVKLSCKASGYTFTDYYIHWVRQAPGQGLEWIGYINPNSGYTNYAQKFQGRATMTADKSINTAYVELSRLRSDDTAVYFCTRYMWERVTGFFDFWGQGTMVTVSS

SEQ ID NO: 43 - protein, 107 aa, synthetic construct
DIQMTQSPSSVSASVGDRVTITCLASEDISDDLAWYQQKPGKAPKVLVYTTSSLQSGVPSRFSGSGSGTDFTLTISSLQPEDFATYFCQQTYKFPPTFGGGTKVEIK

SEQ ID NO: 44 - protein, 116 aa, synthetic construct
EVQLVESGGGLVQPGGSLRLSCAASGFTFNDHWMTWVRQAPGKGLVWVSSITNTGGATYYADSVKGRFTISRDNAKNTLYLQMNSLRAEDTAVYYCTSPGLYFDYWGQGTMVTVSS

SEQ ID NO: 45 - protein, 106 aa, synthetic construct
EIVLTQSPGTLSLSPGERATLSCRASSSVSVMYWYQQKPGQAPRLLIYSTSSLASGIPDRFSGSGSGTDFTLTISRLEPEDFAVYYCHQWSSDPPTFGQGTKLEIK

SEQ ID NO: 46 - protein, 446 aa, synthetic construct
QVQLVQSGAEVKKPGASVKLSCKASGYNFKHTYAHWVRQAPGQGLEWIGRIDPANGNTKYDPKFQGRATMTADTASNTAYLELSSLRSEDTAVYYCVADHYGSSLLDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 47 - protein, 219 aa, synthetic construct
DVVMTQTPLSLSVTPGQPASISCKSSQSLLDSDGKTYLNWFQQRPGQSPRRLIYLVSKLDSGVPDRFSGSGSGTDFTLKISRVEAEDVGVYYCWQGTYFPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 48 - protein, 445 aa, synthetic construct
QVQLVQSGAEVKKPGASVKLSCKASGYTFTDYYINWVRQAPGKGLEWIARIYPGSGNTYYNEKFKGRATLTADKSTSTAYMQLSSLRSEDTAVYFCARGVYYFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAQGGPSVFLFPPKPKDTLYITREPEVTCVVVDVSHEDPEVKFNWYVDGVEVHNAKTKPREEQYNSTYRVVSVLTVLHQDWLNGKEYKCQVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 49 - protein, 214 aa, synthetic construct
DIVMTQSPSSLSASVGDRVTITCKASQNVRTNVAWYQQKPGKAPKALIYSASYRYSGVPDRFTGSGSGTDFTLTISSLQPEDFADYFCQQYNSYPRTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 50 - protein, 447 aa, synthetic construct
QVQLVQSGAEVKKPGASVKVSCKASGYTFTSYNMHWVRQAPGQGLEWMGVIYSGNGDTSYNQKFKGRVTITADKSTSTAYMELSSLRSEDTAVYYCARERDTRFGNWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 51 - protein, 218 aa, synthetic construct
DIVMTQSPDSLAVSLGERATINCRASESVDIYGNSFMHWYQQKPGQPPKLLIYLASNLESGVPDRFSGSGSGTDFTLTISSLQAEDVAVYYCQQNNEDPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 52 - protein, 451 aa, synthetic construct
EVQLVESGGGLVQPGGSLRLSCAASGYSFTGHWMNWVRQAPGKGLEWVGMIHPSDSETRYNQKFKDRFTISVDKSKNTLYLQMNSLRAEDTAVYYCARGIYFYGTTYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 53 - protein, 214 aa, synthetic construct
DIQMTQSPSSLSASVGDRVTITCRASKTISKYLAWYQQKPGKAPKLLIYSGSTLQSGVPSRFSGSGSGTDFTLTISSLQPEDFATYYCQQHNEYPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 54 - protein, 122 aa, synthetic construct
QVQLVQSGAEVKKPGASVKVSCKPSGFNIKDIYMQWVRQAPGQRLEWIGRIDPADDKTKYDPKFQGRATITADTSASTAYLELSSLRSEDTAVYYCASEGHYGYDGYAMDYWGQGTTVTVSS

SEQ ID NO: 55 - protein, 113 aa, synthetic construct
DIVMTQSPDSLAVSLGERATINCKSSQNLLYSSNQKNYLAWYQQKPGQPPKLLIYWASTRESGVPDRFSGSGSGTDFTLTISSLQAEDVAVYYCQQYYSYPLTFGQGTKLEIK

SEQ ID NO: 56 - protein, 446 aa, synthetic construct
EVQLVESGGGLVQPGRSLRLSCTASGFSLNDYWMSWVRQAPGKGLEWVGYIDVGGSLYYAASVKGRFTISRDDSKSIAYLQMNSLKTEDTAVYYCARGGLTYGFDLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFEGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSRLTVDKSRWQQGNVFSCSVMHEALHNHYTQKSLSLSPG

SEQ ID NO: 57 - protein, 217 aa, synthetic construct
DIVMTQSPSSLSASVGDRVTITCQASEDISSYLAWYQQKPGKAPKRLIYGASDLASGVPSRFSASGSGTDYTFTISSLQPEDIATYYCHYYATISGLGVAFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 58 - protein, 450 aa, synthetic construct
EVQLVESGGGLVQPGGSLRLSCAASGFSLSTGGVGVGWIRQAPGKGLEWLALIDWADDKYYSPSLKSRLTISRDNSKNTLYLQLNSLRAEDTAVYYCARGGSDTVIGDWFAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 59 - protein, 213 aa, synthetic construct
DIQLTQSPSSLSASVGDRVTITCRASQSIGSYLAWYQQKPGKAPKLLIYDASNLETGVPSRFSGSGSGTDFTLTISSLQPEDFATYYCQQGYYLWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 60 - protein, 640 aa, synthetic construct
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGGGGSGGGGSGGGGSQDSTSDLIPAPPLSKVPLQQNFQDNQFHGKWYVVGQAGNIRLREDKDPIKMMATIYELKEDKSYDVTMVKFDDKKCMYDIWTFVPGSQPGEFTLGKIKSFPGHTSSLVRVVSTNYNQHAMVFFKFVFQNREEFYITLYGRTKELTSELKENFIRFSKSLGLPENHIVFPVPIDQCIDG

SEQ ID NO: 61 - protein, 214 aa, synthetic construct
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 62 - protein, 451 aa, synthetic construct
QVQLVQSGAEVKKPGASVKVSCKASGYTFTDYIMHWVRQAPGQGLEWMGYINPYNDGSKYTEKFQGRVTMTSDTSISTAYMELSRLRSDDTAVYYCARGTYYYGPQLFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 63 - protein, 219 aa, synthetic construct
DIVMTQTPLSLSVTPGQPASISCKSSQSLETSTGTTYLNWYLQKPGQSPQLLIYRVSKRFSGVPDRFSGSGSGTDFTLKISRVEAEDVGVYYCLQLLEDPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 64 - protein, 116 aa, synthetic construct
QVQLVQSGAEVKKPGASVKVSCKASGYTFAGFEMHWVRQAPGQGLEWMGAIDPKTGGTDYNQKFKDRVTMTRDTSISTAYMELSRLRSDDTAVYYCARDLGYFDVWGQGTLVTVSS

SEQ ID NO: 65 - protein, 108 aa, synthetic construct
DIQMTQSPSSLSASVGDRVTITCRASQDIRSNLNWYQQKPGGAVKLLIYYTSRLHSGVPSRFSGSGSGTDYTLTISSLQPEDFATYFCQQSEKLPRTFGGGTKVEIRR

SEQ ID NO: 66 - protein, 120 aa, synthetic construct [sequence omitted]

SEQ ID NO: 67 - protein, 107 aa, synthetic construct [sequence omitted]

SEQ ID NO: 68 - protein, 446 aa, synthetic construct; identical to SEQ ID NO: 56

SEQ ID NO: 69 - protein, 217 aa, synthetic construct; identical to SEQ ID NO: 57

SEQ ID NO: 70 - protein, 448 aa, synthetic construct
QVQLQQWGAGLLKPSETLSLTCAVYGGSFSGYYWSWIRQSPEKGLEWIGEINHGGYVTYNPSLESRVTISVDTSKNQFSLKLSSVTAADTAVYYCARDYGPGNYDWYFDLWGRGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 71 - protein, 216 aa, synthetic construct
EIVLTQSPATLSLSPGERATLSCRASQSVSSYLAWYQQKPGQAPRLLIYDASNRATGIPARFSGSGSGTDFTLTISSLEPEDFAVYYCQQRSNWPPALTFCGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 72 - protein, 442 aa, synthetic construct
EVQLVQSGAEVKKPGESLRISCKGSGYSFSTYWISWVRQMPGKGLEWMGKIYPGDSYTNYSPSFQGQVTISADKSISTAYLQWSSLKASDTAMYYCARGYGIFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 73 - protein, 214 aa, synthetic construct
SYELTQPPSVSVSPGQTASITCSGDNIGDQYAHWYQQKPGQSPVLVIYQDKNRPSGIPERFSGSNSGNTATLTISGTQAMDEADYYCATYTGFGSLAVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 74 - protein, 457 aa, synthetic construct [sequence omitted]

SEQ ID NO: 75 - protein, 250 aa, synthetic construct
MVAGSDAGRALGVLSVVCLLHCFGFISCFSQQIYGVVYGNVTFHVPSNVPLKEVLWKKQKDKVAELENSEFRAFSSFKNRVYLDTVSGSLTIYNLTSSDEDEYEMESPNITDTMKFFLYVLESLPSPTLTCALTNGSIEVQCMIPEHYNSHRGLIMYSWDCPMEQCKRNSTSIYFKMENDLPQKIQCTLSNPLFNTTSSIILTTCIPSSGHSRHRYALIPIPLAVITTCIVLYMNGILKCDRKPDRTNSN

SEQ ID NO: 76 - protein, 118 aa, synthetic construct
EVQLQQSGPELQRPGASVKLSCKASGYIFTEYYMYWVKQRPKQGLELVGRIDPEDGSIDYVEKFKKKATLTADTSSNTAYMQLSSLTSEDTATYFCARGKFNYRFAYWGQGTLVTVSS

SEQ ID NO: 77 - protein, 112 aa, synthetic construct
DVVLTQTPPTLLATIGQSVSISCRSSQSLLHSSGNTYLNWLLQRTGQSPQPLIYLVSKLESGVPNRFSGSGSGTDFTLKISGVEAEDLGVYYCMQFTHYPYTFGAGTKLELK

SEQ ID NO: 78 - protein, 132 aa, synthetic construct
TNALETWGALGQDIELNIPSFQMSDDIDDIKWEKTSDKKKIAQFRKEKETFKEKDGGSGGLGASWHRPDKFCLGYQKRPLPGGSGGTYELDKNGDLDIKHLKTDDQDIYKVSIYDTKGKNVLEKIFDLKIQE

SEQ ID NO: 79 - protein, 123 aa, synthetic construct
QVQLVQSGAEVKKPGASVKVSCKASGYTFTGYYMHWVRQAPGQGLEWMGRINPNSGGTNYAQKFQGRVTMTRDTSISTAYMELSRLRSDDTAVYYCARGRTEYIVVAEGFDYWGQGTLVTVSS

SEQ ID NO: 80 - protein, 112 aa, synthetic construct
DVVMTQSPPSLLVTLGQPASISCRSSQSLLHSSGNTYLNWLLQRPGQSPQPLIYLVSKLESGVPDRFSGSGSGTDFTLKISGVEAEDVGVYYCMQFTHYPYTFGQGTKLEIK

SEQ ID NO: 81 - protein, 121 aa, synthetic construct
QVQLQQPGAELVRPGASVKLSCKASGYTFTTFWMNWVKQRPGQGLEWIGMIDPSDSEAHYNQMFKDKATLTVDKSSSTAYMQLSSLTSEDSAVYYCARGRGYDDGDAMDYWGQGTSVTVSS

SEQ ID NO: 82 - protein, 111 aa, synthetic construct
DIVMTQSPASLAVSLGQRATISYRASKSVSTSGYSYMHWNQQKPGQPPRLLIYLVSNLESGVPARFSGSGSGTDFTLNIHPVEEEDAATYYCMQFTHYPYTFGGGTKLEIK

SEQ ID NO: 83 - protein, 117 aa, synthetic construct
EVQLEESGGGLVMPGGSLKLSCAASGFAFSSYDMSWVRQTPEKRLEWVAYISGGGFTYYPDTVKGRFTLSRDNAKNTLYLQMSSLKSEDTAMYYCARQGANWELVYWGQGTTLTVSS

SEQ ID NO: 84 - protein, 107 aa, synthetic construct
DIVMTQSPATLSVTPGDRVFLSCRASQSISDFLHWYQQKSHESPRLLIKYASQSISGIPSRFSGSGSGSDFTLSINSVEPEDVGVYLCQNGHNFPPTFGGGTKLEIK

SEQ ID NO: 85 - protein, 121 aa, synthetic construct
QVQLQQPGAELVRPGTSVKLSCKASGYTFTSYWMHWIKQRPEQGLEWIGRIDPYDSETHYNEKFKDKAILSVDKSSSTAYIQLSSLTSDDSAVYYCSRRDAKYDGYALDYWGQGTTLTVSS

SEQ ID NO: 86 - protein, 112 aa, synthetic construct
DIVMTQAAPSVPVTPGESVSISCRSSKTLLHSNGNTYLYWFLQRPGQSPQVLIYRMSNLASGVPNRFSGSGSETTFTLRISRVEAEDVGIYYCMQHLEYPYTFGGGTKLEIK

SEQ ID NO: 87 - protein, 442 aa, synthetic construct
QVQLQQSGSELKKPGASVKISCKASGYSFTDYIILWVRQNPGKGLEWIGHIDPYYGSSNYNLKFKGRVTITADQSTTTAYMELSSLRSEDTAVYYCGRSKRDYFDYWGQGTTLTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 88 - protein, 214 aa, synthetic construct
DIQMTQSPSSLSASIGDRVTITCKASQDINSYLSWFQQKPGKAPKLLIYRANRLVDGVPSRFSGSGSGTDYTLTISSLQPEDFAVYYCLQYDEFPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 89 - protein, 447 aa, synthetic construct
EVQLQQSGTVLARPGASVKMSCKASGYSFTRYWMHWIKQRPGQGLEWIGAIYPGNSDTSYNQKFEGKAKLTAVTSASTAYMELSSLTHEDSAVYYCSRDYGYYFDFWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 90 - protein, 211 aa, synthetic construct
QIVLTQSPAIMSASPGEKVTMTCSASSSISYMQWYQQKPGTSPKRWIYDTSKLASGVPARFSGSGSGTSYSLTISSMEAEDAATYYCHQRSSYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 91 - protein, 445 aa, synthetic construct
QVQLVQSGAEVKKPGSSVKVSCKASGGTFSRYIINWVRQAPGQGLEWMGRIIPILGVENYAQKFQGRVTITADKSTSTAYMELSSLRSEDTAVYYCARKDWFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 92 - protein, 214 aa, synthetic construct
EIVLTQSPGTLSLSPGERATLSCRASQSVSSYLAWYQQKPGQAPRLLIYGASSRATGIPDRFSGSGSGTDFTLTISRLEPEDFAVYYCQQYGSSPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 93 - protein, 446 aa, synthetic construct
QVQLVQSGAEVKKPGSSVKVSCKASGYTFTSYRMHWVRQAPGQGLEWIGYINPSTGYTEYNQKFKDKATITADESTNTAYMELSSLRSEDTAVYYCARGGGVFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 94 - protein, 213 aa, synthetic construct
DIQMTQSPSTLSASVGDRVTITCSASSSISYMHWYQQKPGKAPKLLIYTTSNLASGVPARFSGSGSGTEFTLTISSLQPDDFATYYCHQRSTYPLTFGQGTKVEVKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 95 - protein, 452 aa, synthetic construct
EVQLVESGGGLVQPGGSLRLSCAASGFTFSSFGMHWVRQAPGKGLEWVAYISSDSSAIYYADTVKGRFTISRDNAKNSLYLQMNSLRDEDTAVYYCGRGRENIYYGSRLDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELVGGPSVFLLPPKPKDTLMISRTPEVTCVVVDVSHEDPEVKFNWYVDGVEVHNAKTKPPEEQYNSTLRVVSVLTVLHQDWLNGKEYKCKVSNKALPAPIEKTISKAKGQPREPQVYTLPPSREEMTKNQVSLTCLVKGFYPSDIAVEWESNGQPENNYKTTPLVLDSDGSFFLYSKLTVDKSRWQQGNVFSCSVMHEALHNHYTQKSLSLSPGK

SEQ ID NO: 96 - protein, 214 aa, synthetic construct
DIQLTQSPSFLSASVGDRVTITCKASQNVDTNVAWYQQKPGKAPKALIYSASYRYSGVPSRFSGSGSGTDFTLTISSLQPEDFATYYCQQYNNYPFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 97 - protein, 442 aa, synthetic construct
QVQLQQSGAELVKPGASVKLSCKASGYTFTNYDINWVRQRPEQGLEWIGWIFPGDGSTQYNEKFKGKATLTTDTSSSTAYMQLSRLTSEDSAVYFCARQTTATWFAYWGQGTLVTVSAAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 98 - protein, 214 aa, synthetic construct
DIVMTQSPATLSVTPGDRVSLSCRASQSISDYLHWYQQKSHESPRLLIKYASQSISGIPSRFSGSGSGSDFTLSINSVEPEDVGVYYCQNGHSFPLTFGAGTKLELKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 99 - protein, 504 aa, synthetic construct
DIQLTQSPSFLSASVGDRVTITCKASQNVDTNVAWYQQKPGKAPKALIYSASYRYSGVPSRFSGSGSGTDFTLTISSLQPEDFATYYCQQYNNYPFTFGQGTKLEIKGGGSGGGGEVQLVESGGGLVQPGGSLRLSCAASGFTFSTYAMNWVRQAPGKGLEWVGRIRSKYNNYATYYADSVKDRFTISRDDSKNSLYLQMNSLKTEDTAVYYCVRHGNFGNSYVSWFAYWGQGTLVTVSSGGCGGGEVAALEKEVAALEKEVAALEKEVAALEKGGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 100 - protein, 274 aa, synthetic construct
QAVVTQEPSLTVSPGGTVTLTCRSSTGAVTTSNYANWVQQKPGQAPRGLIGGTNKRAPWTPARFSGSLLGGKAALTITGAQAEDEADYYCALWYSNLWVFGGGTKLTVLGGGGSGGGGEVQLVESGGGLVQPGGSLRLSCAASGFTFSSFGMHWVRQAPGKGLEWVAYISSDSSAIYYADTVKGRFTISRDNAKNSLYLQMNSLRDEDTAVYYCGRGRENIYYGSRLDYWGQGTTVTVSSGGCGGGKVAALKEKVAALKEKVAALKEKVAALKE

SEQ ID NO: 101 - protein, 452 aa, synthetic construct
QVQLVQSGAEVKKPGSSVKVSCKASGYTFTNYVMHWVRQAPGQGLEWMGYINPYNDDVKYNEKFKGRVTITADESTSTAYMELSSLRSEDTAVYYCARWGYYGSPLYYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 102 - protein, 213 aa, synthetic construct
EIVLTQSPATLSLSPGERATLSCRASSRLIYMHWYQQKPGQAPRPLIYATSNLASGIPARFSGSGSGTDFTLTISSLEPEDFAVYYCQQWNSNPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 103 - protein, 446 aa, synthetic construct
EVQLQESGPGLVKPSETLSLTCAVTGYSITSGYSWHWIRQFPGNGLEWMGYIHSSGSTNYNPSLKSRISISRDTSKNQFFLKLSSVTAADTAVYYCAGYDDYFEYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 104 - protein, 214 aa, synthetic construct
DIQMTQSPSSLSASVGDRVTITCKASQNVGFNVAWYQQKPGKSPKALIYSASYRYSGVPSRFSGSGSGTDFTLTISSLQPEDFAEYFCQQYNWYPFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 105 - protein, 115 aa, synthetic construct
QVQLVESGGDLVQPGESLRLSCAASGFIFSDAWMVWVRQAPGKGLEWVGRIKTNGDGGTTDLTEPVKGRFTISRDDSKNMVYLQMNNLRTEDTAIYYCTTAPGFWGQGTLVTVSS

SEQ ID NO: 106 - protein, 114 aa, synthetic construct
DIEMTQSPDSLAYSLGERATINCKSSHNLLYKSNNKNYLAWSQQKPGQPPRLLIYWASTRDSGVPDRFSGSGSGTDFTLTISSLQAEDVAVYYCHQYYGTKWTFGQGTRVEIKR

SEQ ID NO: 107 - protein, 447 aa, synthetic construct
EVQLVESGGGLVKPGGSLRLSCAASGFTFSSYGMSWVRQAPGKGLEWVATINSGGSNTYYPDSLKGRFTISRDNAKNSLYLQMNSLRAEDTAVYYCARHDGGAMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 108 - protein, 215 aa, synthetic construct
DIQMTQSPSSLSASVGDRVTITCRASESIYSYLAWYQQKPGKAPKLLVYNTKTLPEGVPSRFSGSGSGTDFTLTISSLQPEDFATYYCQHHYGTPPWTFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 109 - protein, 452 aa, synthetic construct; identical to SEQ ID NO: 101

SEQ ID NO: 110 - protein, 213 aa, synthetic construct; identical to SEQ ID NO: 102

SEQ ID NO: 111 - protein, 454 aa, synthetic construct
EVQLVESGGGVVRPGGSLRLSCVASGVTFDDYGMSWVRQAPGKGLEWVSGINWNGGDTDYSDSVKGRFTISRDNAKNSLYLQMNSLRAEDTALYYCARDFYGSGSYYHVPFDYWGQGILVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 112 - protein, 215 aa, synthetic construct
EIVLTQSPGTLSLSPGERATLSCRASQSVSRSYLAWYQQKRGQAPRLLIYGASSRATGIPDRFSGDGSGTDFTLSISRLEPEDFAVYYCHQYDMSPFTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 113 - protein, 448 aa, synthetic construct
QVQLVQSGAEVKKPGSSVKVSCKASGYTFTDYAMHWVRQAPGQGLEWMGLISIYSDHTNYNQKFQGRVTITADKSTSTAYMELSSLRSEDTAVYYCGRNNYGNYGWYFDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 114 - protein, 213 aa, synthetic construct
EIVLTQSPATLSLSPGERATLSCSASSSVSYMHWYQQKPGQAPRLLIYDTSKLASGIPARFSGSGSGTDYTLTISSLEPEDFAVYYCFQGSGYPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 115 - protein, 454 aa, synthetic construct
QVQLVQSGAEVKKPGASVKVSCKASGYTFTGYYMHWVRQAPGQGLEWMGWINPHSGGTNYAQKFQGRVTMTRDTSISTAYMELSRLRSDDTAVYYCARTYYYDSSGYYHDAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSDTKVDKKVEPKSCDKTHTCPPCPAPPVAGPSVFLFPPKPKDTLMISRTPEVTCVVVDVKHEDPEVKFNWYVDGVEVHNAKTKPREEEYNSTYRVVSVLTVLHQDWLNGKEYKCKVSNKALPAPIEKTISKAKGQPREPQVYTLPPSREEMTKNQVSLTCDVSGFYPSDIAVEWESDGQPENNYKTTPPVLDSDGSFFLYSKLTVDKSRWEQGDVFSCSVLHEALHSHYTQKSLSLSPGK

SEQ ID NO: 116 - protein, 214 aa, synthetic construct
DIQMTQSPSSVSASVGDRVTITCRASQGISRLLAWYQQKPGKAPKLLIYVASSLQSGVPSRFSGSGSGTDFTLTISSLQPEDFATYYCQQANSFPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 117 - protein, 125 aa, synthetic construct
QVQLVQSGAEVKKPGASVKVSCKASGYTFTGYYMHWVRQAPGQGLEWMGWINPHSGGTNYAQKFQGRVTMTRDTSISTAYMELSRLRSDDTAVYYCARTYYYDSSGYYHDAFDIWGQGTMVTVSS

SEQ ID NO: 118 - protein, 107 aa, synthetic construct
DIQMTQSPSSVSASVGDRVTITCRASQGISRLLAWYQQKPGKAPKLLIYVASSLQSGVPSRFSGSGSGTDFTLTISSLQPEDFATYYCQQANSFPWTFGQGTKVEIK

SEQ ID NO: 119 - protein, 447 aa, synthetic construct
EVQLVESGGGLVQPGGSLRLSCAASGFTFSDYWMDWVRQAPGKGLVWVSNIDEDGSITEYSPFVKGRFTISRDNAKNTLYLQMNSLRAEDTAVYYCTRWGRFGFDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 120 - protein, 218 aa, synthetic construct
DIVMTQSPDSLAVSLGERATINCKSSQSLLSGSFNYLTWYQQKPGQPPKLLIFYASTRHTGVPDRFSGSGSGTDFTLTISSLQAEDVAVYYCHHHYNAPPTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 121 - protein, 363 aa, synthetic construct
DTQEKEVRAMVGSDVELSCACPEGSRFDLNDVYVYWQTSESKTVVTYHIPQHSSLEYVDSRYRNRALMSPAGMLRGDFSLRLFNVTPQDEQKFHCLVLSRSLGFQEVLSVEVTLHVAANFSVGGGGSGGGGSEPKSSDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 122 - protein, 120 aa, synthetic construct
VQLQQSGAELKKPGASVKVSCKASGYTFTEYIIHWIKLRSGQGLEWIGWFYPGSNDIQYNAQFKGKATLTADKSSSTVYMELTGLTPEDSAVYFCARRDDFSGYDALPYWGQGTLVTVSA

SEQ ID NO: 123 - protein, 108 aa, synthetic construct
DIQMTQSPSSLSASVGDRVTITCKTNENIYSNLAWYQQKDGKSPQLLIYAATHLVEGVPSRFSGSGSGTQYSLTISSLQPEDFGNYYCQHFWGTPCTFGGGTKLEIKR

SEQ ID NO: 124 - protein, 108 aa, synthetic construct
DIQMTQSPSSLSASVGDRVTITCRASRPIWPFLEWYQQKPGKAPKLLIYFTSRLRHGVPSRFSGSGSGTCFTLTISSLQPEDFATYYCLQNVANPATFSQGTKVEIKR

SEQ ID NO: 125 - protein, 447 aa, synthetic construct
EVQLVESGGGLVQPGGSLRLSCAASGFTFSSYWMSWVRQAPGKGLEWVAYIKQDGNEKYYVDSVKGRFTISRDNAKNSLYLQMNSLRAEDTAVYYCAREGILWFGDLPTFWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 126 - protein, 214 aa, synthetic construct [sequence omitted]

SEQ ID NO: 127 - protein, 452 aa, synthetic construct
QVQLVESGGGVVQPGRSLRLSCAASGFTFSSYDMHWVRQAPGKGLEWVAVIWYDGSNKYYADSVKGRFTISRDNSKNTLYLQMNSLRAEDTAVYYCARGSGNWGFFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGSS

SEQ ID NO: 128 - protein, 214 aa, synthetic construct
DIQMTQSPSSLSASVGDRVTITCRASQGISRWLAWYQQKPEKAPKSLIYAASSLQSGVPSRFSGSGSGTDFTLTISSLQPEDFATYYCQQYNTYPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 129 - protein, 345 aa, synthetic construct
VIHVTKEVKEVATLSCGYNVSVEELEQTRIYWQKDKKMVLTMMSGDLNIWPEYKNRTIFDITNNLSIMILGLRPSDEGTYECVVLKYEKGAFKREHLAEVTLSVKADGSGGGGSEPKSSDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 130 - protein, 119 aa, synthetic construct
QVQLQQSGPELVKPGASVKISCKASGYTFTNYYIHWVKQRPGQGLEWIGWIYPGDGNTKYNEKFKGKATLTADTSSSTAYMQLSSLTSEDSAVYYCARDSYSNYYFDYWGQGTTLTVSS

SEQ ID NO: 131 - protein, 112 aa, synthetic construct
DIVMTQSPSSLAVSAGEKVTMSCKSSQSLLNSRTRKNYLAWYQQKPGQSPKLLIYWASTRESGVPDRFTGSGSGTDFTLTISSVQAEDLAVYYCTQSFILRTFGGGTKLEIK

SEQ ID NO: 132 - protein, 249 aa, synthetic construct
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 133 - protein, 446 aa, synthetic construct; identical to SEQ ID NO: 32

SEQ ID NO: 134 - protein, 232 aa, synthetic construct
EVQLLESGGGLVQPGGSLRLSCAASGFTFSTYAMNWVRQAPGKGLEWVSRIRSKYNNYATYYADSVKGRFTISRDDSKNTLYLQMNSLRAEDTAVYYCVRHGNFGNSYVSWFAYWGQGTLVTVSSASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 135 - protein, 504 aa, synthetic construct
DIQLTQSPASLAVSLGQRATISCKASQSVDYDGDSYLNWYQQIPGQPPKLLIYDASNLVSGIPPRFSGSGSGTDFTLNIHPVEKVDAATYHCQQSTEDPWTFGGGTKLEIKGGGGSGGGGSGGGGSQVQLQQSGAELVRPGSSVKISCKASGYAFSSYWMNWVKQRPGQGLEWIGQIWPGDGDTNYNGKFKGKATLTADESSSTAYMQLSSLASEDSAVYFCARRETTTVGRYYYAMDYWGQGTTVTVSSGGGGSDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELKHHHHHH

SEQ ID NO: 136 - protein, 449 aa, synthetic construct
EVQLVQSGAEVKKPGASVKVSCKASGFTFTSYYIHWVRQAPGQGLEWIGWIYPENDNTKYNEKFKDRVTITADTSTSTAYLELSSLRSEDTAVYYCARDGYSRYYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 137 - protein, 219 aa, synthetic construct
DIVMTQSPDSLAVSLGERATINCKSSQSLLNSRTRKNYLAWYQQKPGQSPKLLIYWTSTRKSGVPDRFSGSGSGTDFTLTISSLQAEDVAVYYCKQSFILRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 138 - protein, 693 aa, synthetic construct
QVQLVQSGAEVKKPGASVKVSCKASGYTFTEFGMNWVRQAPGQGLEWMGWINTKTGEATYVEEFKGRVTFTTDTSTSTAYMELRSLRSDDTAVYYCARWDFAYYVEAMDYWGQGTTVTVSSATKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDGGGGSGGGGSEVQLLESGGGLVQPGGSLRLSCAASGFTFSTYAMNWVRQAPGKGLEWVSRIRSKYNNYATYYADSVKGRFTISRDDSKNTLYLQMNSLRAEDTAVYYCVRHGNFGNSYVSWFAYWGQGTLVTVSSASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 139 - protein, 214 aa, synthetic construct
QAVVTQEPSLTVSPGGTVTLTCGSSTGAVTTSNYANWVQEKPGQAFRGLIGGTNKRAPGTPARFSGSLLGGKAALTLSGAQPEDEAEYYCALWYSNLWVFGGGTKLTVLSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 140 - protein, 456 aa, synthetic construct
QVQLQQSGAELARPGASVKMSCKASGYTFTSYTMHWVKQRPGQGLEWIGYINPSSGYTNYIQRFKDKATLTADKSSSTAYMQVSSLTSEDSAVYYCARGSRYDYYGMDYWGQGTSVTVSSAKTTPPSVYPLAPGCGDTTGSSVTLGCLVKGYFPESVTVTWNSGSLSSSVHTFPALLQSGLYTMSSSVTVPSSTWPSQTVTCSVAHPASSTTVDKKLEPSGPISTINPCPPCKECHKCPAPNLEGGPSVFIFPPNIKDVLMISLTPKVTCVVVDVSEDDPDVQISWFVNNVEVHTAQTQTHREDYNSTIRVVSTLPIQHQDWMSGKEFKCKVNNKDLPSPIERTISKIKGLVRAPQVYILPPPAEQLSRKDVSLTCLVVGFNPGDISVEWTSNGHTEENYKDTAPVLDSDGSYFIYSKLNMKTSKWEKTDSFSCNVRHEGLKNYYLKKTISRSPGK

SEQ ID NO: 141 - protein, 213 aa, synthetic construct
QIVLTQSPAIMSASPGEKVTMTCSASSSVSYMHWYQQKSGTSPKRWIYDTSKLASGVPARFSGSGSGTSYSLTISSMEAEDAATYYCQQWSSNPLTFGAGTKLELKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 142 - protein, 502 aa, synthetic construct
ENVLTQSPATLSVTPGEKATITCRASQSVSYMHWYQQKPGQAPRLLIYDASNRASGVPSRFSGSGSGTDHTLTISSLEAEDAATYYCFQGSVYPFTFGQGTKLEIKGGGSGGGGEVQLVESGGGLVQPGGSLRLSCAASGFTFSTYAMNWVRQAPGKGLEWVGRIRSKYNNYATYYADSVKGRFTISRDDSKNSLYLQMNSLKTEDTAVYYCVRHGNFGNSYVSWFAYWGQGTLVTVSSASTKGEVAACEKEVAALEKEVAALEKEVAALEKGGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 143 - protein, 270 aa, synthetic construct
QAVVTQEPSLTVSPGGTVTLTCRSSTGAVTTSNYANWVQQKPGQAPRGLIGGTNKRAPWTPARFSGSLLGGKAALTITGAQAEDEADYYCALWYSNLWVFGGGTKLTVLGGGGSGGGGQVTLRESGPALVKPTQTLTLTCTFSGFSLSTSGMGVGWIRQPPGKALEWLAHIWWDDDKRYNPALKSRLTISKDTSKNQVFLTMTNMDPVDTATYYCARMELWSYYFDYWGQGTTVTVSSASTKGKVAACKEKVAALKEKVALKEKVAALKE

SEQ ID NO: 144 - protein, 447 aa, synthetic construct
EVQLVESGGGLVQPGGSLRLSCAASGFTFSDYYMTWVRQAPGKGLEWVAFIRNRARGYTSDHNPSVKGRFTISRDNAKNSLYLQMNSLRAEDTAVYYCARDRPSYYVLDYWGQGTTVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCRVRCPRCPAPPVAGPSVFLFPPKPKDTLMISRTPEVTCVVVAVSHEDPEVQFNWYVDGVEVHNAKTKPREEQFNSTFRVVSVLTVVHQDWLNGKEYKCKVSNKGLPSSIEKTISKTKGQPREPQVYTLPPSREEMTKNQVSLTCLVKGFYPSDIAVEWESNGQPENNYKTTPPMLDSDGSFFLYSRLTVDKSRWQQGNVFSCSVMHEALHNHYTQKSLSLSPGK

SEQ ID NO: 145 - protein, 219 aa, synthetic construct
DIVMTQSPDSLAVSLGERATINCKSSQSLFNVRSRKNYLAWYQQKPGQPPKLLISWASTRESGVPDRFSGSGSGTDFTLTISSLQAEDVAVYYCKQSYDLFTFGSGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 146 - protein, 511 aa, synthetic construct
QVQLVQSGAEVKKPGESVKVSCKASGYTFTNYGMNWVKQAPGQGLEWMGWINTYTGEPTYADKFQGRVTMTTDTSTSTAYMEIRNLGGDDTAVYYCARWSWSDGYYVYFDYWGQGTSVTVSSGGGGSGGGGSGGGGSDIVMTQSPDSLTVSLGERTTINCKSSQSVLDSSTNKNSLAWYQQKPGQPPKLLLSWASTRESGIPDRFSGSGSGTDFTLTIDSPQPEDSATYYCQQSAHFPITFGQGTRLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLHHHHHH

SEQ ID NO: 147 - protein, 993 aa, synthetic construct
QVQLVQSGAEVKKPGESVKVSCKASGYTFTNYGMNWVKQAPGQCLEWMGWINTYTGEPTYADKFQGRVTMTTDTSTSTAYMEIRNLGGDDTAVYYCARWSWSDGYYVYFDYWGQGTSVTVSSGGGGSGGGGSGGGGSDIVMTQSPDSLTVSLGERTTINCKSSQSVLDSSTNKNSLAWYQQKPGQPPKLLLSWASTRESGIPDRFSGSGSGTDFTLTIDSPQPEDSATYYCQQSAHFPITFGCGTRLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 148 - protein, 1230 aa, synthetic construct
DVVMTQSPSTLSASVGDRVTINCQASESISSWLAWYQQKPGKAPKLLIYEASKLASGVPSRFSGSGSGTEFTLTISSLQPDDFATYYCQGYFYFISRTYVNSFGGGTKVEIKGGGGSGGGGSGGGGSGGGGSEVQLVESGGGLVQPGGSLRLSCAASGFTISTNAMSWVRQAPGKGLEWIGVITGRDITYYASWAKGRFTISRDNSKNTLYLQMNSLRAEDTAVYYCARDGGSSAITSNNIWGQGTLVTVSSGGGGSGGGGSEVQLVQSGAEVKKPGESLKISCKGSGYSFSSSWIGWVRQAPGKGLEWMGIIYPDDSDTRYSPSFQGQVTISADKSIRTAYLQWSSLKASDTAMYYCARHVTMIWGVIIDFWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSEVQLLESGGGLVQPGGSLRLSCAASGFSFSSGYDMCWVRQAPGKGLEWIACIAAGSAGITYDANWAKGRFTISRDNSKNTLYLQMNSLRAEDTAVYYCARSAFSFDYAMDLWGQGTLVTVSSGGGGSGGGGSGGGGSGGGGSDIQMTQSPSTLSASVGDRVTITCQASQSISSHLNWYQQKPGKAPKLLIYKASTLASGVPSRFSGSGSGTEFTLTISSLQPDDFATYYCQQGYSWGNVDNVFGGGTKVEIKGGGGSGGGGSRSLVESGGGLVQPGGSLRLSCTASGFTISSYHMQWVRQAPGKGLEYIGTISSGGNVYYASSARGRFTISRPSSKNTVDLQMNSLRAEDTAVYYCARDSGYSDPMWGQGTLVTVSSGGGGSGGGGSGGGGSGGGGSDVVMTQSPSSVSASVGDRVTITCQASQNIRTYLSWYQQKPGKAPKLLIYAAANLASGVPSRFSGSGSGTDFTLTISDLEPGDAATYYCQSTYLGTDYVGGAFGGGTKVEIK

SEQ ID NO: 149 - protein, 214 aa, synthetic construct
AIQLTQSPSSLSASVGDRVTITCRASQGISSALAWYQQKPGKAPKLLIYDASSLESGVPSRFSGSGSGTDFTLTISSLQPEDFATYYCQQFNSYPFTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 150 - protein, 989 aa, synthetic construct
QVTLKESGPTLVKPTETLTLTCTLSGFSLNNARMGVSWIRQPPGKCLEWLAHIFSNDEKSYSTSLKNRLTISKDSSKTQVVLTMTNVDPVDTATYYCARIVGYGSGWYGFFDYWGQGTLVTVSSGGGGSGGGGSGGGGSDIQMTQSPSSLSASVGDRVTITCRASQGIRNDLGWYQQKPGKAPKRLIYAASTLQSGVPSRFSGSGSGTEFTLTISSLQPEDFATYYCLQHNSYPLTFGCGTKVEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 151 - protein, 451 aa, synthetic construct
EVQLVESGGGLVQPDRSLRLSCAASGFTFHDYAMHWVRQAPGKGLEWVSTISWNSGTIGYADSVKGRFTISRDNAKNSLYLQMNSLRAEDTALYYCAKDIQYGNYYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFEGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSRLTVDKSRWQQGNVFSCSVMHEALHNHYTQKSLSLSPG

SEQ ID NO: 152 - protein, 214 aa, synthetic construct
EIVLTQSPATLSLSPGERATLSCRASQSVSSYLAWYQQKPGQAPRLLIYDASNRATGIPARFSGSGSGTDFTLTISSLEPEDFAVYYCQQRSNWPITFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 153 - protein, 512 aa, synthetic construct
QVQLVESGGGVVQSGRSLRLSCAASGFTFRNYGMHWVRQAPGKCLEWVAVIWYDGSDKYYADSVRGRFTISRDNSKNTLYLQMNSLRAEDTAVYYCARDGYDILTGNPRDFDYWGQGTLVTVSSGGGGSGGGGSGGGGSDTVMTQTPLSSHVTLGQPASISCRSSQSLVHSDGNTYLSWLQQRPGQPPRLLIYRISRRFSGVPDRFSGSGAGTDFTLEISRVEAEDVGVYYCMQSTHVPRTFGCGTKVEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLHHHHHH

SEQ ID NO: 154 - protein, 123 aa, synthetic construct
EVQLVESGGGLVQPGRSLRLSCAASGFTFDDYAMHWVRQAPGKGLEWVSGISWNSGSIGYADSVKGRFTISRDNAKNSLYLQMNSLRAEDTALYYCAKDSRGYGDYRLGGAYWGQGTLVTVSS

SEQ ID NO: 155 - protein, 107 aa, synthetic construct
EIVMTQSPATLSVSPGERATLSCRASQSVSSNLAWYQQKPGQAPRLLIYGASTRATGIPARFSGSGSGTEFTLTISSLQSEDFAVYYCQQYNNWPWTFGQGTKVEIK

SEQ ID NO: 156 - protein, 280 aa, synthetic construct
DFVMTQSPDSLAVSLGERVTMSCKSSQSLLNSGNQKNYLTWYQQKPGQPPKLLIYWASTRESGVPDRFSGSGSGTDFTLTISSLQAEDVAVYYCQNDYSYPYTFGQGTKLEIKGGGSGGGGEVQLVESGGGLVQPGGSLRLSCAASGFTFSTYAMNWVRQAPGKGLEWVGRIRSKYNNYATYYADSVKDRFTISRDDSKNSLYLQMNSLKTEDTAVYYCVRHGNFGNSYVSWFAYWGQGTLVTVSSGGCGGGKVAALKEKVAALKEKVAALKEKVAALKE

SEQ ID NO: 157 - protein, 272 aa, synthetic construct
QAVVTQEPSLTVSPGGTVTLTCRSSTGAVTTSNYANWVQQKPGQAPRGLIGGTNKRAPWTPARFSGSLLGGKAALTITGAQAEDEADYYCALWYSNLWVFGGGTKLTVLGGGGSGGGGEVQLVQSGAELKKPGASVKVSCKASGYTFTDYYMKWVRQAPGQGLEWIGDIIPSNGATFYNQKFKGRVTITVDKSTSTAYMELSSLRSEDTAVYYCARSHLLRASWFAYWGQGTLVTVSSGGCGGGEVAALEKEVAALEKEVAALEKEVAALEK

SEQ ID NO: 158 - protein, 448 aa, synthetic construct
QVQLVESGGGVVQPGRSLRLSCAASGFKFSGYGMHWVRQAPGKGLEWVAVIWYDGSKKYYVDSVKGRFTISRDNSKNTLYLQMNSLRAEDTAVYYCARQMGYWHFDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 159 - protein, 215 aa, synthetic construct
EIVLTQSPATLSLSPGERATLSCRASQSVSSYLAWYQQKPGQAPRLLIYDASNRATGIPARFSGSGSGTDFTLTISSLEPEDFAVYYCQQRSNWPPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 160 - protein, 449 aa, synthetic construct
QVQLVQSGAEVKKPGSSVKVSCKASGYAFSYSWINWVRQAPGQGLEWMGRIFPGDGDTDYNGKFKGRVTITADKSTSTAYMELSSLRSEDTAVYYCARNVFDGYWLVYWGQGTLVTVSSASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDEKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 161 - protein, 232 aa, synthetic construct; identical to SEQ ID NO: 134

SEQ ID NO: 162 - protein, 990 aa, synthetic construct
QVQLVQSGAEVKKPGASVKVSCKASGYTFTGYYMHWVRQAPGQCLEWMGWINPNSGGTKYAQKFQGRVTMTRDTSISTAYMELSRLRSDDTAVYYCARDRITVAGTYYYYGMDVWGQGTTVTVSSGGGGSGGGGSGGGGSDIQMTQSPSSVSASVGDRVTITCRASQGVNNWLAWYQQKPGKAPKLLIYTASSLQSGVPSRFSGSGSGTDFTLTIRSLQPEDFATYYCQQANSFPITFGCGTRLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 163 - protein, 125 aa, synthetic construct
EVQLVESGGGLVQPGGSLRLSCAASGFTFNTYAMNWVRQAPGKGLEWVGRIRSKYNNYATYYADSVKDRFTISRDNSKNTLYLQMNSLRAEDTAVYYCVRHGNFGNSYVSWFAYWGQGTLVTVSS

SEQ ID NO: 164 - protein, 109 aa, synthetic construct
EAVVTQEPSLTVSPGGTVTLTCRSSTGAVTTSNYANWFQQKPGQAPRTLIYGTNKRAPWTPARFSGSLLGGKAALTLSGAQPEDEAEYYCALWYSNLWVFGGGTKLTVL

SEQ ID NO: 165 - protein, 119 aa, synthetic construct
EVQLVQSGAEVKKPGASVKVSCKASGFTFTSYYIHWVRQAPGQGLEWIGWIYPENDNTKYNEKFKDRVTITADTSTSTAYLELSSLRSEDTAVYYCARDGYSRYYFDYWGQGTLVTVSS

SEQ ID NO: 166 - protein, 112 aa, synthetic construct
DIVMTQSPDSLAVSLGERATINCKSSQSLLNSRTRKNYLAWYQQKPGQSPKLLIYWTSTRKSGVPDRFSGSGSGTDFTLTISSLQAEDVAVYYCKQSFILRTFGQGTKVEIK

SEQ ID NO: 167 - protein, 118 aa, synthetic construct
EVQLVESGGGLVQPGGSLRLSCAASGFNIKDTYIHFVRQAPGKGLEWIGRIDPANDNTLYASKFQGKATISADTSKNTAYLQMNSLRAEDTAVYYCGRGYGYYVFDHWGQGTLVTVSS

SEQ ID NO: 168 - protein, 107 aa, synthetic construct
DVQITQSPSSLSASVGDRVTITCRTSRSISQYLAWYQQKPGKVPKLLIYSGSTLQSGVPSRFSGSGSGTDFTLTISSLQPEDVATYYCQQHNENPLTFGGGTKVEIK

SEQ ID NO: 169 - protein, 993 aa, synthetic construct
QVQLVQSGAEVKKPGASVKVSCKASGYTFISYTMHWVRQAPGQGLEWMGYINPRSGYTHYNQKLKDKATLTADKSASTAYMELSSLRSEDTAVYYCARSAYYDYDGFAYWGQGTLVTVSSGGGGSGGGGSGGGGSDIQMTQSPSSLSASVGDRVTITCSASSSVSYMNWYQQKPGKAPKRLIYDTSKLASGVPSRFSGSGSGTDFTLTISSLQPEDFATYYCQQWSSNPPTFGGGTKVEIKGGGGSGGGGSGGGGSQEDERIVLVDNKCKCARITSRIIRSSEDPNEDIVERNIRIIVPLNNRENISDPTSPLRTRFVYHLSDLCKKCDPTEVELDNQIVTATQSNICDEDSATETCYTYDRNKCYTAVVPLVYGGETKMVETALTPDACYPDGGGGSGGGGSGGGGSDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFKQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 170 - protein, 118 aa, synthetic construct
DIKLQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSS

SEQ ID NO: 171 - protein, 106 aa, synthetic construct
DIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 172 - protein, 450 aa, synthetic construct
EVQLVESGGGLVQPGRSLRLSCAASGFTFDDYSMHWVRQAPGKGLEWVSGISWNSGSKGYADSVKGRFTISRDNAKNSLYLQMNSLRAEDTALYYCAKYGSGYGKFYHYGLDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNRFTQKSLSLSPGK

SEQ ID NO: 173 - protein, 449 aa, synthetic construct
EVQLVQSGAEVKKPGASVKVSCKASGYTFTNYYIHWVRQAPGQGLEWIGWIYPGDGNTKYNEKFKGRATLTADTSTSTAYLELSSLRSEDTAVYYCARDSYSNYYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 174 - protein, 219 aa, synthetic construct
DIVMTQSPDSLAVSLGERATINCKSSQSLLNSRTRKNYLAWYQQKPGQPPKLLIYWASTRESGVPDRFSGSGSGTDFTLTISSLQAEDVAVYYCTQSFILRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 175 - protein, 450 aa, synthetic construct
QVQLQQSGAELARPGASVKMSCKASGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSAKTTAPSVYPLAPVCGGTTGSSVTLGCLVKGYFPEPVTLTWNSGSLSSGVHTFPAVLQSDLYTLSSSVTVTSSTWPSQSITCNVAHPASSTKVDKKIEPRPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 176 - protein, 213 aa, synthetic construct
QIVLTQSPAIMSASPGEKVTMTCSASSSVSYMNWYQQKSGTSPKRWIYDTSKLASGVPAHFRGSGSGTSYSLTISGMEAEDAATYYCQQWSSNPFTFGSGTKLEINRADTAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 177 - protein, 484 aa, synthetic construct
EIVMTQTPATLSVSAGERVTITCKASQSVSNDVTWYQQKPGQAPRLLIYSASNRYSGVPARFSGSGYGTEFTFTISSVQSEDFAVYFCQQDYSSFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECTSGGGGSGGGGSGGGGSQVQLVQSGGGVVQPGRSLRLSCKASGYTFTRYTMHWVRQAPGKCLEWIGYINPSRGYTNYNQKFKDRFTISRDNSKNTAFLQMDSLRPEDTGVYFCARYYDDHYSLDYWGQGTPVTVSSGGGGSGGGGSGGGGSGGGGSGGGGSGGGGSDIQMTQSPSSLSASVGDRVTITCSASSSVSYMNWYQQTPGKAPKRWIYDTSKLASGVPSRFSGSGSGTDYTFTISSLQPEDIATYYCQQWSSNPFTFGCGTKLQITR

SEQ ID NO: 178 - protein, 504 aa, synthetic construct; identical to SEQ ID NO: 99

SEQ ID NO: 179 - protein, 274 aa, synthetic construct; identical to SEQ ID NO: 100

SEQ ID NO: 180 - protein, 449 aa, synthetic construct
EVQLVESGGGLVQPGRSLRLSCAASGFTFDDYTMHWVRQAPGKGLEWVSGISWNSGSIGYADSVKGRFTISRDNAKKSLYLQMNSLRAEDTALYYCAKDNSGYGHYYYGMDVWGQGTTVTVASASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNRFTQKSLSLSLGK

SEQ ID NO: 181 - protein, 449 aa, synthetic construct
EVQLLESGGGLVQPGGSLRLSCAASGFTFSSFPMAWVRQAPGKGLEWVSTISTSGGRTYYRDSVKGRFTISRDNSKNTLYLQMNSLRAEDTAVYYCAKFRQYSGGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 182 - protein, 216 aa, synthetic construct
DIQLTQPNSVSTSLGSTVKLSCTLSSGNIENNYVHWYQLYEGRSPTTMIYDDDKRPDGVPDRFSGSIDRSSNSAFLTIHNVAIEDEAIYFCHSYVSSFNVFGGGTKLTVLRQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 183 - protein, 504 aa, synthetic construct
QVQLVQSGAEVKKPGASVKVSCKASGYTFTNHIIHWVRQAPGQGLEWMGYINPYPGYHAYNEKFQGRATMTSDTSTSTVYMELSSLRSEDTAVYYCARDGYYRDTDVLDYWGQGTLVTVSSGGGGSGGGGSGGGGSDIQMTQSPSSLSASVGDRVTITCQASQDISNYLNWYQQKPGKAPKLLIYYTSRLHTGVPSRFSGSGSGTDFTFTISSLEPEDIATYYCQQGNTLPWTFGQGTKVEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLHHHHHH

SEQ ID NO: 184 - protein, 504 aa, synthetic construct
QVQLVESGGGLVKPGESLRLSCAASGFTFSDYYMYWVRQAPGKGLEWVAIISDGGYYTYYSDIIKGRFTISRDNAKNSLYLQMNSLKAEDTAVYYCARGFPLLRHGAMDYWGQGTLVTVSSGGGGSGGGGSGGGGSDIQMTQSPSSLSASVGDRVTITCKASQNVDTNVAWYQQKPGQAPKSLIYSASYRYSDVPSRFSGSASGTDFTLTISSVQSEDFATYYCQQYDSYPYTFGGGTKLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLHHHHHH

SEQ ID NO: 185 - protein, 986 aa, synthetic construct; identical to SEQ ID NO: 40

SEQ ID NO: 186 - protein, 485 aa, synthetic construct
EVQLVESGGGLVQPGGSLRLSCAASGFTFSTYAMNWVRQAPGKGLEWVGRIRSKYNNYATYYADSVKGRFTISRDDSKNTLYLQMNSLRAEDTAVYYCVRHGNFGDSYVSWFAYWGQGTLVTVSSGKPGSGKPGSGKPGSGKPGSQAVVTQEPSLTVSPGGTVTLTCGSSTGAVTTSNYANWVQQKPGKSPRGLIGGTNKRAPGVPARFSGSLLGGKAALTISGAQPEDEADYYCALWYSNHWVFGGGTKLTVLEPKSSDKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 187 - protein, 449 aa, synthetic construct; identical to SEQ ID NO: 173

SEQ ID NO: 188 - protein, 219 aa, synthetic construct; identical to SEQ ID NO: 174

SEQ ID NO: 189 - protein, 502 aa, synthetic construct
ELVMTQSPSSLTVTAGEKVTMSCKSSQSLLNSGNQKNYLTWYQQKPGQPPKLLIYWASTRESGVPDRFTGSGSGTDFTLTISSVQAEDLAVYYCQNDYSYPLTFGAGTKLEIKGGGGSGGGGSGGGGSEVQLLEQSGAELVRPGTSVKISCKASGYAFTNYWLGWVKQRPGHGLEWIGDIFPGSGNIHYNEKFKGKATLTADKSSSTAYMQLSSLTFEDSAVYFCARLRNWDEPMDYWGQGTTVTVSSGGGGSDVQLVQSGAEVKKPGASVKVSCKASGYTFTRYTMHWVRQAPGQGLEWIGYINPSRGYTNYADSVKGRFTITTDKSTSTAYMELSSLRSEDTATYYCARYYDDHYCLDYWGQGTTVTVSSGEGTSTGSGGSGGSGGADDIVLTQSPATLSLSPGERATLSCRASQSVSYMNWYQQKPGKAPKRWIYDTSKVASGVPARFSGSGSGTDYSLTINSLEAEDAATYYCQQWSSNPLTFGGGTKVEIKHHHHHH

SEQ ID NO: 190 - protein, 125 aa, synthetic construct
EVQLVESGGGLVQPKGSLKLSCAASGFTFNTYAMNWVRQAPGKGLEWVARIRSKYNNYATYYADSVKDRFTISRDDSQSILYLQMNNLKTEDTAMYYCVRHGNFGNSYVSWFAYWGQGTLVTVSA

SEQ ID NO: 191 - protein, 109 aa, synthetic construct
QAVVTQESALTTSPGETVTLTCRSSTGAVTTSNYANWVQEKPDHLFTGLIGGTNKRAPGVPARFSGSLIGDKAALTITGAQTEDEAIYFCALWYSNLWVFGGGTKLTVL

SEQ ID NO: 192 - protein, 452 aa, synthetic construct
EVQLVESGGGLVQPGGSLRLSCAASGFTFNTYAMNWVRQAPGKGLEWVARIRSKYNNYATYYAASVKGRFTISRDDSKNSLYLQMNSLKTEDTAVYYCARHGNFGNSYVSWFAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFLLYSKLTVDKSRWQEGNVFSCSVMHEALHNHYTQKSLSLSLGK

SEQ ID NO: 193 - protein, 215 aa, synthetic construct
QTVVTQEPSLTVSPGGTVTLTCRSSTGAVTTSNYANWVQQKPGQAPRGLIGGTNKRAPGTPARFSGSLLGGKAALTLSGVQPEDEAEYYCALWYSNLWVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 194 - protein, 982 aa, synthetic construct
QVQLQESGPGLVKPSETLSLTCTVSGGSISSYYWSWIRQPPGKCLEWIGYVYYSGTTNYNPSLKSRVTISVDTSKNQFSLKLSSVTAADTAVYYCASIAVTGFYFDYWGQGTLVTVSSGGGGSGGGGSGGGGSEIVLTQSPGTLSLSPGERVTLSCRASQRVNNNYLAWYQQRPGQAPRLLIYGASSRATGIPDRFSGSGSGTDFTLTISRLEPEDFAVYYCQQYDRSPLTFGCGTKLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 195 - protein, 500 aa, synthetic construct
AIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGQGTKVEIKGGGGSGGGGSGGGGSGGGGSGGGSEVQLVESGGGLVQPGGSLRLSCAASGYSFTGYTMNWVRQAPGKGLEWVALINPYKGVSTYNQKFKDRFTISVDKSKNTAYLQMNSLRAEDTAVYYCARSGYYGDSDWYFDVWGQGTLVTVSSGGGGSDGGITQSPKYLFRKEGQNVTLSCEQNLNHDAMYWYRQDPGQGLRLIYYSWAQGDFQKGDIAEGYSVSREKKESFPLTVTSAQKNPTAFYLCASSWGAPYEQYFGPGTRLTVTEDLKNVFPPEVAVFEPSEAEISHTQKATLVCLATGFYPDHVELSWWVNGKEVHSGVCTDPQPLKEQPALNDSRYALSSRLRVSATFWQDPRNHFRCQVQFYGLSENDEWTQDRAKPVTQIVSAEAWGRAD

SEQ ID NO: 196 - protein, 122 aa, synthetic construct [sequence omitted]

SEQ ID NO: 197 - protein, 107 aa, synthetic construct
AIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGQGTKVEIK

SEQ ID NO: 198 - protein, 452 aa, synthetic construct; identical to SEQ ID NO: 192

SEQ ID NO: 199 - protein, 215 aa, synthetic construct; identical to SEQ ID NO: 193

SEQ ID NO: 200 - protein, 449 aa, synthetic construct
QVQLVQSGGGVVQPGRSLRLSCKASGYTFTRYTMHWVRQAPGKGLEWIGYINPSRGYTNYNQKVKDRFTISRDNSKNTAFLQMDSLRPEDTGVYFCARYYDDHYCLDYWGQGTPVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 201 - protein, 213 aa, synthetic construct
DIQMTQSPSSLSASVGDRVTITCSASSSVSYMNWYQQTPGKAPKRWIYDTSKLASGVPSRFSGSGSGTDYTFTISSLQPEDIATYYCQQWSSNPFTFGQGTKLQITRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 202 - protein, 447 aa, synthetic construct
QVQLVQSGGGVVQPGRSLRLSCVASGFTFSSYGMHWVRQAPGKGLEWVAAIWYNARKQDYADSVKGRFTISRDNSKNTLYLQMNSLRAEDTAVYYCTRGTGYNWFDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELGRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTKPPSREEMTKNQVSLKCLVKGFYPSDIAVEWESNGQPENNYKTTPPVLDSDGSFFLYSKLTVDKSRWQQGNVFSCSVMHEALHNHYTQKSLSLSPG

SEQ ID NO: 203 - protein, 485 aa, synthetic construct; identical to SEQ ID NO: 186

SEQ ID NO: 204 - protein, 117 aa, synthetic construct
KLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSS

SEQ ID NO: 205 - protein, 106 aa, synthetic construct
IVLTQSPSIMSVSPGEKVTITCSASSSVSYMHWFQQKPGTSPKLSIYSTSNLASGVPARFSGRGSGTSYSLTISRVAAEDAATYYCQQRSNYPPWTFGGGTKLEIK

SEQ ID NO: 206 - protein, 450 aa, synthetic construct; identical to SEQ ID NO: 172

SEQ ID NO: 207 - protein, 122 aa, synthetic construct [sequence omitted]

SEQ ID NO: 208 - protein, 107 aa, synthetic construct; identical to SEQ ID NO: 197

SEQ ID NO: 209 - protein, 984 aa, synthetic construct
QVQLQQWGAGLLKPSETLSLTCAVYGGSFSGYYWSWIRQPPGKCLEWIGDIDASGSTKYNPSLKSRVTISLDTSKNQFSLKLNSVTAADTAVYFCARKKYSTVWSYFDNWGQGTLVTVSSGGGGSGGGGSGGGGSSYELTQPSSVSVPPGQTASITCSGDKLGDKYASWYQQKPGQSPVLVIYQDRKRPSGVPERFSGSNSGNTATLTISGTQAMDEADYYCQAWGSSTAVFGCGTKLTVLSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 210 - protein, 485 aa, synthetic construct; identical to SEQ ID NO: 186

SEQ ID NO: 211 - protein, 446 aa, synthetic construct
QVQLVQSGAEVKKPGASVKVSCKASGYTFISYTMHWVRQAPGQGLEWMGYINPRSGYTHYNQKLKDKATLTADKSASTAYMELSSLRSEDTAVYYCARSAYYDYDGFAYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPAAAPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPSK

SEQ ID NO: 212 - protein, 213 aa, synthetic construct
DIQMTQSPSSLSASVGDRVTITCSASSSVSYMNWYQQKPGKAPKRLIYDTSKLASGVPSRFSGSGSGTDFTLTISSLQPEDFATYYCQQWSSNPPTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 213 - protein, 483 aa, synthetic construct
DIQMTQSPSSLSASVGDRVTITCRSSTGAVTTSNYANWVQQKPGKAPKALIGGTNKRAPGVPSRFSGSLIGDKATLTISSLQPEDFATYYCALWYSNLWVFGQGTKVEIKGGSGGSQVQLVQSGAEVKKPGASVKVSCKASGYTFTSYDINWVRQAPGQGLEWMGWMNPNSGNTGFAQKFQGRVTMTRDTSTSTVYMELSSLRSEDTAVYYCARDRANTDYSLGMDVWGQGTLVTVSSGGSGQSVLTQPPSASGTPGQRVTISCSGSRSNIGSNTVNWYQQLPGTAPKLLIYGNNQRPSGVPDRFSGSKSGTSASLAISGLQSEDEADYYCATWDDSLIGWVFGGGTKLTVLGGSGGSEVQLVESGGGLVQPGGSLRLSCAASGFTFSTYAMNWVRQAPGKGLEWVGRIRSKYNNYATYYADSVKDRFTISRDDSKNSLYLQMNSLKTEDTAVYYCARHGNFGNSYVSYFAYWGQGTLVTVSS

SEQ ID NO: 214 - protein, 452 aa, synthetic construct; identical to SEQ ID NO: 192

SEQ ID NO: 215 - protein, 215 aa, synthetic construct; identical to SEQ ID NO: 193

SEQ ID NO: 216 - protein, 125 aa, synthetic construct
EVQLVESGGGLVQPGGSLRLSCAASGFTFNTYAMNWVRQAPGKGLEWVARIRSKYNNYATYYADSVKDRFTISRDDSKNSLYLQMNSLKTEDTAVYYCVRHGNFGNSYVSWFAYWGQGTLVTVSS

SEQ ID NO: 217 - protein, 109 aa, synthetic construct
QAVVTQEPSLTVSPGGTVTLTCRSSTGAVTTSNYANWVQQKPGQAPRGLIGGTNKRAPWTPARFSGSLLGGKAALTITGAQAEDEADYYCALWYSNLWVFGGGTKLTVL

SEQ ID NO: 218 - protein, 450 aa, synthetic construct; identical to SEQ ID NO: 175

SEQ ID NO: 219 - protein, 213 aa, synthetic construct; identical to SEQ ID NO: 176

SEQ ID NO: 220 - protein, 449 aa, synthetic construct
QVQLQQSGPEVVKPGASVKMSCKASGYTFTSYVIHWVRQKPGQGLDWIGYINPYNDGTDYDEKFKGKATLTSDTSTSTAYMELSSLRSEDTAVYYCAREKDNYATGAWFAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 221 - protein, 219 aa, synthetic construct
DIVMTQSPDSLAVSLGERVTMNCKSSQSLLYSTNQKNYLAWYQQKPGQSPKLLIYWASTRESGVPDRFSGSGSGTDFTLTISSVQAEDVAVYYCQQYYSYRTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 222 - protein, 698 aa, synthetic construct
EVQLLESGGGLVQPGGSLRLSCAASGFTFSRNAMSWVRQAPGKGLEWVSATGGSGISTYYADSVKGRFTISRDNSKNTLYLQMNSLRAEDTAVYYCARGYSNSWWYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGSGGGGSQVQLVESGGGLVKPGGSLRLSCAASGFTFSDYYMSWIRQAPGKCLEWISYISSSESIIYYVDAVKGRFTISRDNAKNSLYLQMNSLRAEDTAVYYCARDVGSHFDYWGQGTLVTVSSGGGGSGGGGSGGGGSDIQMTQSPSSVSASVGDRVTITCRASQDISRWLAWYQQKPGKAPKLLISAASRLQSGVPSRFSGSGSGTDFTLTISSLQPEDFAIYYCQQAKSFPRTFGCGTKVEIKR

SEQ ID NO: 223 - protein, 217 aa, synthetic construct
QSALTQPASVSGSPGQSITISCTGTSSDVGNYNLVSWYQQHPGKAPKLMIFEVNQRPSGVSNRFSGSKSGTTASLTISGLQAADEADYFCSSYTTSSTYVIFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 224 - protein, 448 aa, synthetic construct
QVQLVQSGPELKKPGASVKVSCKASGYTFTDYVIHWVKQATGQGLEWIGEIYPGSGSAYSNAKFKDRVTMTADKSSNTAYMELSSLTSDDTAVYFCARRGNGTGFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYHSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 225 - protein, 218 aa, synthetic construct
DIVLTQSPASLAVSLGQRATITCKAGQSVDYDGDSYMNWYQQKPGQPPKLLIYVASNLESGIPARFSGSGSGTDFTLNIHPVEENDAATYYCQQSYKDPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKLYACEVTHQGLSSPVTKSFNRGEC

SEQ ID NO: 226 - protein, 454 aa, synthetic construct
EEQLVESGGGLVKPGGSLRLSCAASGFSFSDCRMYWLRQAPGKGLEWIGVISVKSENYGANYAESVRGRFTISRDDSKNTVYLQMNSLKTEDTAVYYCSASYYRYDVGAWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 227 - protein, 218 aa, synthetic construct
DIVMTQSPDSLAVSLGERATINCRASKSVSTSGYSYIYWYQQKPGQPPKLLIYLASILESGVPDRFSGSGSGTDFTLTISSLQAEDVAVYYCQHSRELPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 228 - protein, 448 aa, synthetic construct
QLQLQESGPGLLKPSETLSLTCTVSGGSISSPGYYGGWIRQPPGKGLEWIGSIYKSGSTYHNPSLKSRVTISVDTSKNQFSLKLSSVTAADTAVYYCTRPVVRYFGWFDPWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 229 - protein, 213 aa, synthetic construct
AIQLTQSPSSLSASVGDRVTITCRASQGISSALAWYQQKPGKAPKLLIYDASNLESGVPSRFSGSGSGTDFTLTISSLQPEDFATYYCQQFNSYPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 230 - protein, 444 aa, synthetic construct
EVQLVESGGGLVQPGGSLRLSCAASGYSFTGYYIHWVRQAPGKGLEWVARVIPNAGGTSYNQKFKGRFTLSVDNSKNTAYLQMNSLRAEDTAVYYCAREGIYWWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 231 - protein, 219 aa, synthetic construct
DIQMTQSPSSLSASVGDRVTITCRSSQSLVHSNGNTFLHWYQQKPGKAPKLLIYTVSNRFSGVPSRFSGSGSGTDFTLTISSLQPEDFATYFCSQTTHVPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 232 - protein, 444 aa, synthetic construct; identical to SEQ ID NO: 230

SEQ ID NO: 233 - protein, 219 aa, synthetic construct; identical to SEQ ID NO: 231

SEQ ID NO: 234 - protein, 442 aa, synthetic construct
EVQLQESGPGLVKPSETLSLTCTVSGYSITSNYYWNWIRQPPGKGLEWMGYIRYDGSNNYNPSLKNRVTISRDTSKNQFSLKLSSVTAADTAVYYCARLDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEEKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 235 - protein, 219 aa, synthetic construct
DIVMTQTPLSLSVTPGQPASISCRSSQSLENTNGNTFLNWYLQKPGQSPQLLIYRVSNRFSGVPDRFSGSGSGTDFTLKISRVEAEDVGVYYCLQVTHVPFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 236 - protein, 450 aa, synthetic construct
QVQLVESGGGVVQPGRSLRLSCAASGFTFSSYGMHWVRQAPGKGLEWVAVISYEESNRYHADSVKGRFTISRDNSKITLYLQMNSLRTEDTAVYYCARDGGIAAPGPDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 237 - protein, 219 aa, synthetic construct
DIVMTQSPLSLTVTPGEPASISCRSSQSLLYSNGYNYLDWYLQKPGQSPQVLISLGSNRASGVPDRFSGSGSGTDFTLKISRVEAEDVGVYYCMQARQTPFTFGPGTKVDIRRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 238 - protein, 450 aa, synthetic construct
QVQLVESGGGVVQPGRSLRLSCAASGFTFSSYGMHWVRQAPGKGLEWVAVISYEESNRYHADSVKGRFTISRDNSKITLYLQMNSLRTEDTAVYYCARDGGIAAPGPDYWGQGTLVTVSSASTKGPSVFPLAPASKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 239 - protein, 219 aa, synthetic construct; identical to SEQ ID NO: 237

SEQ ID NO: 240 - protein, 449 aa, synthetic construct
EVQLLESGGGLVQPGGSLRLSCAASGFTFSTYGMHWVRQAPGKGLEWLSYISGGSSYIFYADSVRGRFTISRDNSENALYLQMNSLRAEDTAVYYCARILRGGSGMDLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 241 - protein, 217 aa, synthetic construct
QSVLTQPPSASGTPGQRVTISCTGSSSNIGAGYNVYWYQQLPGTAPKLLIYGNINRPSGVPDRFSGSKSGTSASLAISGLRSEDEADYYCAAWDKSISGLVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 242 - protein, 446 aa, synthetic construct
EVQLVESGGGLVKPGGSLRLSCAASGFTFSDYGMNWVRQAPGKGLEWIAYISSGRGNIYYADTVKGRFTISRDNAKNSLYLQMNSLRAEDTAVYYCARSWGYFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDQLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHNHYTQKSLSLSPGK

SEQ ID NO: 243 - protein, 220 aa, synthetic construct
DIVMTQSPDSLAVSLGERATINCKSSQSLLNRGNQKNYLTWFQQKPGQPPKLLIYWASTRESGVPDRFSGSGSGTDFTLTISSLQAEDVAVYYCQNDYTYPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 244 - protein, 452 aa, synthetic construct
QVQLVQSGAEVKKPGASVKVSCKASGYTFTGYYMHWVRQAPGQGLEWMGWINPDSGGTNYAQKFQGRVTMTRDTSISTAYMELNRLRSDDTAVYYCARDQPLGYCTNGVCSYFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 245 - protein, 214 aa, synthetic construct
DIQMTQSPSSVSASVGDRVTITCRASQGIYSWLAWYQQKPGKAPNLLIYTASTLQSGVPSRFSGSGSGTDFTLTISSLQPEDFATYYCQQANIFPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 246 - protein, 450 aa, synthetic construct
QVQLVESGGGVVQPGRSLRLSCAASGFSFSSTYVCWVRQAPGKGLEWIACIYTGDGTNYSASWAKGRFTISKDSSKNTVYLQMNSLRAEDTAVYFCARPDITYGFAINFWGPGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVEHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 247 - protein, 215 aa, synthetic construct
DIQMTQSPSSLSASVGDRVTIKCQASQSISSRLAWYQQKPGKPPKLLIYRASTLASGVPSRFSGSGSGTDFTLTISSLQPEDVATYYCQCTGYGISWPIGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 248 - protein, 450 aa, synthetic construct
EVQLVQSGAEVKKPGASVKVSCKTSGYTFTEYIMHWVRQAPGQGLEWMGGIIPNNGGTSYNQKFQGRVTMTVDKSTSTGYMELSSLRSEDTAVYYCTRREVYGRNYYALDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFEGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFLLYSKLTVDKSRWQQGNVFSCSVMHEALHNHYTQKSLSLSP

SEQ ID NO: 249 - protein, 214 aa, synthetic construct
DIQMTQSPSSLSASVGDRVTITCSASQGINNYLNWYQQKPGKAVKLLIYYTSSLHSGVPSRFSGSGSGTDYTFTISSLQPEDIATYYCQQYSNLPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 250 - protein, 452 aa, synthetic construct
QIQLVQSGPELKKPGETVRISCKASGYAFTTTGMQWVQEMPGKGLKWIGWINTHSGVPKYVEDFKGRFAFSLETSANTAYLQISNLKNEDTATYFCVRSGNGNYDLAYFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 251 - protein, 214 aa, synthetic construct
DIVLTQSPATLSVTPGDRVSLSCRASQSISDYLHWYQQKSHESPRLLIKYASHSISGIPSRFSGSGSGSDFTLSINSVEPEDVGIYYCQHGHSFPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 252 - protein, 229 aa, synthetic construct
EVQLVESGGGLVQPGGSLRLSCAVSGFSSTNYHVHWVRQAPGKGLEWMGVIWGDGDTSYNSVLKSRFTISRDTSKNTVYLQMNSLRAEDTAVYYCARQLTHYYVLAAWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCAA

SEQ ID NO: 253 - protein, 214 aa, synthetic construct
DIQMTQSPSSLSASVGDRVTITCRASEDLYYNLAWYQRKPGKAPKLLIYDTYRLADGVPSRFSGSGSGTDYTLTISSLQPEDFASYYCQQYYKFPFTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 254 - protein, 450 aa, synthetic construct
EVQLQESGPGLVKPSETLSLTCTVSGDSITNGFWIWIRKPPGNKLEYMGYISYSGSTYYNPSLKSRISISRDTSKNQFSLKLSSVTAADTGVYYCAYRSYGRTPYYFDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKAEPKSCDKTHTCPPCPAPELLGGPSVFLFPPKPKDTLMISRTPEVTCVVVDVSHRDPEVKFNWYVDGVEVHNAKTKPREEQYNSTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 255 - protein, 214 aa, synthetic construct
DIVMTQSPSFLSASVGDRVTITCKASSNLGHAVAWYQQKPGKSPKLLIYSASNRYTGVPDRFSGSGSGTDFTLTISSLQPEDFADYFCQQYDDYPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 256 - protein, 353 aa, synthetic construct
EVQLLESGGGLVQPGGSLRLSCAASGFTFNWELMGWARQAPGKGLEWVSGIEGPGDVTYYADSVKGRFTISRDNSKNTLYLQMNSLRAEDTAVYYCVKVGKDAKSDYRGQGTLVTVSSASTEPKSSDKTHTSPPSPAPELLGGSSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 257 - protein, 447 aa, synthetic construct
QVQLVQSGAEVVKPGASVKLSCKASGYIFTSYYMYWVKQAPGQGLEWIGEINPSNGDTNFNEKFKSKATLTVDKSASTAYMELSSLRSEDTAVYYCTRSDGRNDMDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 258 - protein, 218 aa, synthetic construct
DIVLTQSPATLSVSPGERATISCRASQRVSSSTYSYMHWYQQKPGQPPKLLIKYASNLESGVPARFSGSGSGTDFTLTISSVEPEDFATYYCQHSWEIPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 259 - protein, 118 aa, synthetic construct
QIQLVQSGPELKKPGETVKISCKASGYTFTNYGMNWVKQAPGKGLRWMGWINTHTGEPTYADDFKGRFAFSLETSASTAYLQINNLKNEDTATYFCTRRGYDWYFDVWGAGTTVTVSS

SEQ ID NO: 260 - protein, 107 aa, synthetic construct
DIKMTQSPSSMYASLGERVTITCKASQDINSYLSWFQQKPGKSPKTLIYRANRLVDGVPSRFSGSGSGQDYSLTISSLDYEDMGIYYCQQYDESPWTFGGGTKLEIK

SEQ ID NO: 261 - protein, 120 aa, synthetic construct
QVTLKESGPGILKPSQTLSLTCSFSGFSLSTSGMGVGWIRQPSGKGLEWLAHIWWDDDVYYNPSLKNQLTISKDASRDQVFLKITNLDTADTATYYCVRRRATGTGFDYWGQGTTLTVSS

SEQ ID NO: 262 - protein, 95 aa, synthetic construct
NIVMTQSHKFMSTSVGDRVSIACKASQDVGTAVAWYQQKPGQSPKLLIYWTSTRHTGVPDRFTGSGSGTDFTLTISNVQSEDLADYFCQQYSSYP

SEQ ID NO: 263 - protein, 448 aa, synthetic construct
QVQLVESGGGVVQPGRSLRLSCAASGFTFSSFGMHWVRQAPGKGLEWVAYISSGSFTIYYADSVKGRFTISRDNSKNTLYLQMNSLRAEDTAVYYCARMRKGYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 264 - protein, 219 aa, synthetic construct
DVVMTQSPLSLPVTLGQPASISCRSSQIIIHSDGNTYLEWFQQRPGQSPRRLIYKVSNRFSGVPDRFSGSGSGTDFTLKISRVEAEDVGVYYCFQGSHVPHTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 265 - protein, 120 aa, synthetic construct
QVKLQESGGGLVKPGGSLKLSCAASGFTFSSYAMSWVRQTPEKRLEWVATISSGGSYTYYPDSVKGRFTISRDNAKNTLYLQMSSLRSEDTAMYYCARQDGYYPGWFANWGQGTTVTVSS

SEQ ID NO: 266 - protein, 105 aa, synthetic construct
DIELTQSPAIMSASLGEEITLTCSASSSVSYMHWYQQKSGTSPKLLIYSTSNLASGVPSRFSGSGSGTFYSLTISSVEAEDAADYYCHQWSSYTFGGGTKLEIKR

SEQ ID NO: 267 - protein, 123 aa, synthetic construct
QVQLQESGAELVKPGASVKLSCKASGYTFTSYWMHWVKQRPGQGLEWIGKINPSNGRTNYNEKFKSKATLTVDKSSSTAYMQLSSLTSEDSAVYYCARGGVYYDLYYYALDYWGQGTTVTVSS

SEQ ID NO: 268 - protein, 108 aa, synthetic construct
DIELTQSPATLSVTPGDSVSLSCRASQSISNNLHWYQQKSHESPRLLIKSASQSISGIPSRFSGSGSGTDFTLSINSVETEDFGMYFCQQSNSWPYTFGGGTKLEIKR

SEQ ID NO: 269 - protein, 453 aa, synthetic construct
QIQLVQSGPELKKPGETVKISCKASGYTFTNYGMNWVKQAPGKGLMWLGWINTYTGEPTYADDFKGRFAFSLETSASTAYLQINNLKNEDTATYFCARWAYFYGSSPYFFDYWGQGTTLTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 270 - protein, 215 aa, synthetic construct
QAVVTQESALTTSPGETVTLTCRSSTGAVTTSNYANWVQEKPDHLFTGLIGGTNNRAPGVPARFSGSLIGDKAALTITGAQTEDEAIYFCALWCSNHLVFGGGTKLTVLGQPKSSPSVTLFPPSSEELETNKATLVCTITDFYPGVVTVDWKVDGTPVTQGMETTQPSKQSNNKYMASSYLTLTARAWERHSSYSCQVTHEGHTVEKSLSRADCS

SEQ ID NO: 271 - protein, 125 aa, synthetic construct
DVQLQESGGGLVQAGGSLRLSCAVSGYPYSSYCMGWFRQAPGKEREGVAAIDSDGRTRYADSVKGRFTISQDNAKNTLYLQMNRMKPEDTAMYYCAARFGPMGCVDLSTLSFGHWGQGTQVTVSS

SEQ ID NO: 272 - protein, 123 aa, synthetic construct
QIQLVQSGPELKKPGETVKISCKASGYTFTNYGMNWVKQAPGKGLMWLGWINTYTGEPTYADDFKGRFAFSLETSASTAYLQINNLKNEDTATYFCARWAYFYGSSPYFFDYWGQGTTLTVSS

SEQ ID NO: 273 - protein, 109 aa, synthetic construct
QAVVTQESALTTSPGETVTLTCRSSTGAVTTSNYANWVQEKPDHLFTGLIGGTNNRAPGVPARFSGSLIGDKAALTITGAQTEDEAIYFCALWCSNHLVFGGGTKLTVL

SEQ ID NO: 274 - protein, 123 aa, synthetic construct; identical to SEQ ID NO: 272

SEQ ID NO: 275 - protein, 109 aa, synthetic construct; identical to SEQ ID NO: 273

SEQ ID NO: 276 - protein, 452 aa, synthetic construct
EVQLVESGGGLVQPGGSLRLSCAASGFTFSVYYMNWVRQAPGKGLEWVSDINNEGGTTYYADSVKGRFTISRDNSKNSLYLQMNSLRAEDTAVYYCARDAGYSNHVPIFDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 277 - protein, 216 aa, synthetic construct
QAVVTQEPSLTVSPGGTVTLTCGLKSGSVTSDNFPTWYQQTPGQAPRLLIYNTNTRHSGVPDRFSGSILGNKAALTITGAQADDEAEYFCALFISNPSVEFGGGTQLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 278 - protein, 448 aa, synthetic construct
QVQLVQSGAEVKKPGASVKVSCKASGYTFTNYGMNWVRQAPGQGLKWMGWINTYTGEPTYADAFKGRVTMTRDTSISTAYMELSRLRSDDTAVYYCARDYGDYGMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 279 - protein, 218 aa, synthetic construct
DIVMTQSPDSLAVSLGERATINCRASKSVSTSGYSFMHWYQQKPGQPPKLLIYLASNLESGVPDRFSGSGSGTDFTLTISSLQAEDVAVYYCQHSREVPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 280 - protein, 603 aa, synthetic construct
EVQLVQSGAEVKKPGESLKISCKASGYAFSSSWINWVRQMPGKGLEWMGRIYPRAGDTNYAGKFKDQVTISADKSISTAYLQWSSLKASDTAMYYCASLLDYSMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSGGGGSGIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 281 - protein, 214 aa, synthetic construct
DIQMTQSPSSLSASVGDRVTITCRASQDISNYLNWYQQKPGKAPKLLIYYTSRLHSGVPSRFSGSGSGTDFTFTISSLQPEDIATYYCQQGNTLPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 282 - protein, 451 aa, synthetic construct
QVQLVQSGAEVVKPGASVKVSCKASGYSFTGYTMNWVRQAPGQNLEWIGLINPYNAGTSYNQKFQGKVTLTVDKSTSTAYMELSSLRSEDTAVYYCARSEYRYGGDYFDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 283 - protein, 220 aa, synthetic construct
DIVMTQSPSSLAVSVGERVTISCKSSQSLLNSSNQKNYLAWYQQKPGQAPKLLIYFASTRESGVPDRFSGSGSGTDFTLTISSLQAEDVAVYYCQQHYDTPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 284 - protein, 452 aa, synthetic construct
QVQLVQSGAEVEKPGASVKVSCKASGYTFTSYWITWVRQAPGQGLEWMGDIYPGSGNTNYNEKFKTRVTITADKSTSTAYMELSSLRSEDTAVYYCAKEGGLTTEDYALDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 285 - protein, 218 aa, synthetic construct
EIVLTQSPATLSLSPGERATLSCRASKNVSTSGYSYMHWYQQKPGQAPRLLIYLASNLESGIPPRFSGSGYGTDFTLTINNIESEDAAYYFCQHSRELPFTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 286 - protein, 447 aa, synthetic construct
EVQLLESGGGLVQPGGSLRLSCAASGFTFSSYAYSWVRQAPGKGLEWVSAISGSGGRTYYADSVKGRFTISRDNSKNTLYLQMNSLRAEDTAVYYCARLGYGRVDEWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 287 - protein, 216 aa, synthetic construct
QSVLTQPPSASGTPGQRVTISCSGSLSNIGRNPVNWYQQLPGTAPKLLIYLDNLRLSGVPDRFSGSKSGTSASLAISGLQSEDEADYYCATWDDSHPGWTFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 288 - protein, 450 aa, synthetic construct
EVQLQESGPGLVKPSETLSLTCAVSGYSITSGYYWNWIRQPPGKKLEWMGYINYGGSNGYNPSLKSRITISRDTSKNQFSLKLSSVTAADTAVYYCARDYDAYYEALDDWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 289 - protein, 213 aa, synthetic construct
EIVLSQSPATLSLSPGERATLSCRASSRVNYMHWYQQKPGQSPRPWISATSNLASGVPARFSGSGSGTSYTLTISSLEPEDFAVYYCQQWSSNPPTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 290 - protein, 113 aa, synthetic construct
QVQLVESGGDVVQPGGSLRLSCAASGVAFSNYGMHWVRQAPGKGLEWVAVIWYDGSNKYYADSVKGRFTISRDNSKNMLYLQMNSLRAEDTAMYYCARNDDYWGQGTLVTVSS

SEQ ID NO: 291 - protein, 107 aa, synthetic construct
EIVLTQSPATLSLSPGERATLSCRASQSVSSYLAWYQQKPGQAPRLIIYDASNRATGIPARFSGSGSGTDFTLTISSLEPEDFAVYYCQQRSNWPLTFGGGTKVEIK

SEQ ID NO: 292 - protein, 118 aa, synthetic construct; identical to SEQ ID NO: 167

SEQ ID NO: 293 - protein, 107 aa, synthetic construct; identical to SEQ ID NO: 168

SEQ ID NO: 294 - protein, 118 aa, synthetic construct
EVQLVESGGGLVQPGGSLRLSCAASGFNIKDTYIHWVRQAPGKGLEWVARIDPANDNTLYASKFQGRATISADTSKNTAYLQMNSLRAEDTAVYYCGRGYGYYVFDHWGQGTLVTVSS

SEQ ID NO: 295 - protein, 118 aa, synthetic construct
QVQLVQSGAEVKKPGATVKISCKVSGFNIKDTYIHWVQQAPGKGLEWMGRIDPANDNTLYASKFQGRVTITADTSTDTAYMELSSLRSEDTAVYYCARGYGYYVFDHWGQGTLVTVSS

SEQ ID NO: 296 - protein, 120 aa, synthetic construct
QVQLQQPGAELVRPGTSVKLSCRASGYTFTSYWMHWVKQRPGQGLEWIGVIDPSDSYTNYNQKFRGKATLTVDTSSSTAYMQLSSLTSEDSAVYYCARPYYGSHWFFDVWGTGTTVTVSS

SEQ ID NO: 297 - protein, 106 aa, synthetic construct
QIVLTQSPAIMSASPGEKVTMTCSASSRVSYVHWYQQKSGTSPKRWLYDTSNLASGVPARFGGNGSGTSYSLTISSMEAEDAATYYCQQWSTNPPTFGGGTKLEIK

SEQ ID NO: 298 - protein, 713 aa, synthetic construct
EVQLVESGGGLVQPGGSLRLSCAASGFAFSSYDMSWVRQAPGKGLDWVATISGGGRYTYYPDSVKGRFTISRDNSKNNLYLQMNSLRAEDTALYYCANRYGEAWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSQVQLVESGAEVKKPGASVKVSCKASGYSFTGYTMNWVRQAPGQCLEWIGLINPYNNITNYAQKFQGRVTFTVDTSISTAYMELSRLRSDDTGVYFCARLDYRSYWGQGTLVTVSAGGGGSGGGGSGGGGSGGGGSQAVVTQEPSLTVSPGGTVTLTCGSSTGAVTTSNFPNWVQQKPGQAPRSLIGGTNNKASWTPARFSGSLLGGKAALTISGAQPEDEAEYYCALWYSNHWVFGCGTKLTVLR

SEQ ID NO: 299 - protein, 493 aa, synthetic construct
QVQLVESGGGLVQPGGSLRLSCAASGKMSSRRCMAWFRQAPGKERERVAKLLTTSGSTYLADSVKGRFTISRDNSKNTVYLQMNSLRAEDTAVYYCAADSFEDPTCTLVTSSGAFQYWGQGTLVTVSSGAPQVQLVESGGGLVQPGGSLRLSCAASGYIYSAYCMGWFRQAPGKGLEGVAAIYIGGGSTYYADSVKGRFTISRDNSKNTLYLQMNSLRAEDTAVYYCAADVIPTETCLGGSWSGPFGYWGQGTLVTVSSGSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 300 - protein, 499 aa, synthetic construct
EIVLTQSPATLSLSPGERATLSCRASESVDNYGMSFMNWFQQKPGQPPKLLIHAASNQGSGVPSRFSGSGSGTDFTLTISSLEPEDFAVYFCQQSKEVPYTFGGGTKVEIKGGGSGGGGQVQLVESGGGVVQPGRSLRLSCAASGFTFSSYTMHWVRQAPGKGLEWVTFISYDGSNKHYADSVKGRFTVSRDNSKNTLYLQMNSLRAEDTAIYYCARTGWLGPFDYWGQGTLVTVSSGGCGGGEVAACEKEVAALEKEVAALEKEVAALEKESKYGPPCPPCPAPEFLGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 301 - protein, 269 aa, synthetic construct
EIVLTQSPGTLSLSPGERATLSCRASQSVSSSFLAWYQQKPGQAPRLLIYGASSRATGIPDRFSGSGSGTDFTLTISRLEPEDFAVYYCQQYGSSPWTFGQGTKVEIKGGGSGGGGQVQLVQSGAEVKKPGASVKVSCKASGYSFTSYWMNWVRQAPGQGLEWIGVIHPSDSETWLDQKFKDRVTITVDKSTSTAYMELSSLRSEDTAVYYCAREHYGTSPFAYWGQGTLVTVSSGGCGGGKVAACKEKVAALKEKVAALKEKVAALKE

SEQ ID NO: 302 - protein, 447 aa, synthetic construct
EVQLVESGGGLVKPGGSLRLSCAASGFTFSSYTMHWVRQAPGKGLEWVSFISYDGNNKYYADSVKGRFTISRDNAKNSLYLQMNSLRAEDTAVYYCARTGWLGPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSDTKVDKKVEPKSCDKTHTCPPCPAPPVAGPSVFLFPPKPKDTLMISRTPEVTCVVVDVKHEDPEVKFNWYVDGVEVHNAKTKPREEEYNSTYRVVSVLTVLHQDWLNGKEYKCKVSNKALPAPIEKTISKAKGQPREPQVYTLPPSREEMTKNQVSLTCDVSGFYPSDIAVEWESDGQPENNYKTTPPVLDSDGSFFLYSKLTVDKSRWEQGDVFSCSVLHEALHSHYTQKSLSLSPGK

SEQ ID NO: 303 - protein, 215 aa, synthetic construct
EIVLTQSPGTLSLSPGERATLSCRASQSVSSSYLAWYQQKPGQAPRLLIYGAFSRATGIPDRFSGSGSGTDFTLTISRLEPEDFAVYYCQQYGSSPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 304 - protein, 450 aa, synthetic construct
EVQLVESGGGLVQPGGSLRLSCAASGFTFDDAWMSWVRQAPGKGLEWVAEISTKANNHATYYAESVKGRFTISRDDSKSSVYLQMNSLRAEDTAVYYCTRLATWDWYFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSDTKVDKKVEPKSCDKTHTCPPCPAPPVAGPSVFLFPPKPKDTLMISRTPEVTCVVVDVKHEDPEVKFNWYVDGVEVHNAKTKPREEEYNSTYRVVSVLTVLHQDWLNGKEYKCKVSNKALPAPIEKTISKAKGQPREPQVYTLPPSREEMTKNQVSLTCDVSGFYPSDIAVEWESDGQPENNYKTTPPVLDSDGSFFLYSKLTVDKSRWEQGDVFSCSVLHEALHSHYTQKSLSLSPGK

SEQ ID NO: 305 - protein, 218 aa, synthetic construct
DIVLTQSPSSLSASVGDRVTITCRASQSVDYDGDSYMNWYQQKPGKPPKLLIYAASELESGIPARFSGSGSGTDFTLTISSLQPEDFATYYCQQSNEDPFTFGSGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 306 - protein, 447 aa, synthetic construct
EVQLVESGGGLVKPGGSLRLSCAASGFTFSSYSMNWVRQAPGKGLEWVSSISSSSSYIYYAESVKGRFTISRDNAKNSLYLQMNSLRAEDTAVYYCARVGLFGPFDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPLPEEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 307 - protein, 214 aa, synthetic construct
EIVLTQSPGTLSLSPGERATLSCRASQSVSRYLGWYQQKPGQAPRLLIYGASTRATGIPDRFSGSGSGTDFTLTITRLEPEDFAVYYCQQYGSSPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 308 - protein, 713 aa, synthetic construct; identical to SEQ ID NO: 298

SEQ ID NO: 309 - protein, 214 aa, synthetic construct
DIQMTQSPSSMSASVGDRVTFTCRASQDINTYLSWFQQKPGKSPKTLIYRANRLVSGVPSRFSGSGSGQDYTLTISSLQPEDMATYYCLQYDEFPLTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 310 - protein, 448 aa, synthetic construct; identical to SEQ ID NO: 16

SEQ ID NO: 311 - protein, 215 aa, synthetic construct; identical to SEQ ID NO: 17

SEQ ID NO: 312 - protein, 123 aa, synthetic construct
EVKLVNSGGGLVQPGNSLRLSCATSGFTFTDFYMSWVRQPPGKALEWLGFVRNRANGYTTEYSVSVKGRFIISRDNFQSTLFLQMNTLRAEDSGTYYCVRGPGDTADYTMDYWGQGTSVTVSS

SEQ ID NO: 313 - protein, 107 aa, synthetic construct
QAVVTQESAFTTSPGGTVILTCRSSTGAVTTNNYANWVQEKPDHLFTGLIGGTSNRAPGVPVRFSGSLIGDKAALTITGAQTEDDGMYFCALWYTTHFVFGGGTKVT

SEQ ID NO: 314 - protein, 446 aa, synthetic construct
EVQLVESGGGVVQPGRSLRLSCAASGFTFSSYTMHWVRQAPGKGLEWVTFISYDGNNKYYADSVKGRFTISRDNSKNTLYLQMNSLRAEDTAIYYCARTGWLGPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 315 - protein, 215 aa, synthetic construct; identical to SEQ ID NO: 17

SEQ ID NO: 316 - protein, 445 aa, synthetic construct
EVQLVESGGGLVQPGGSLRLSCAASGFTFSDNWMNWVRQAPGKGLEWLAQIRNKPYNYETYYSASVKGRFTISRDDSKNSVYLQMNSLKTEDTGVYYCTAQFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 317 - protein, 214 aa, synthetic construct
DIQMTQSPSSLSASVGDRVTITCRTSENIYGGLNWYQRKPGKSPKLLIYGATNLASGVSSRFSGSGSGTDYTLTISSLQPEDVATYYCQNVLRSPFTFGSGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 318 - protein, 451 aa, synthetic construct
QVQLVESGGGVVQPGRSLRLSCAASGFTFSSYGMHWVRQAPGKGLEWVAVIWYDGSNKYYADSVKGRFTISRDNSKNTLYLQMNSLRAEDTAVYYCARDPRGATLYYYYYGMDVWGQGTTVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 319 - protein, 214 aa, synthetic construct
DIQMTQSPSSLSASVGDRVTITCRASQSINSYLDWYQQKPGKAPKLLIYAASSLQSGVPSRFSGSGSGTDFTLTISSLQPEDFATYYCQQYYSTPFTFGPGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 320 - protein, 448 aa, synthetic construct
QVQLVESGGGVVEPGRSLRLSCAASGFTFSSYGMHWVRQAPGKGLEWVAVIWYKPSEKDYADSAKGRFTISRDNSKNTLYLQMNSLRAEDTAVYYCARGGLLGYFDYWGQGTLVTVSSASTKGPSVFPLAPSSKSTSGGTAALGCLVDDYFPEPVTVSWNSGALTSGVHTCPACLQSSGLYSLSSVVTVPSSSLGTQTYICNVNHKPSNTKVDKKVEPKSGDKTHTCPPCPAPELLGGPSVFLFPPKPKDTLMISKTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLRSDGSFFLYSELTVDKSRWQQGNVFSCSVMHEALHNHYTQKSLSLSPGK

SEQ ID NO: 321 - protein, 214 aa, synthetic construct
EIVLTQSPGTLSLSPGERATLSCRASQSINSYLAWYQQKPGQAPRPLIYGVSSRATGIPDRFSGSGSGTDFTLTISRLEPEDFAVYYCQQYGRYPFTFGPGTKVDIKRTVAAPSVFIFPPSDEQLKSGTAKVVCLLNNFYPREAKVQWKVDNALQSGNSCECVTEQDSKDSTYSLSSTLTLSKADYEKHKVYACEVTHQGLSSPVTKSFNRGES

SEQ ID NO: 322 - protein, 115 aa, synthetic construct
QVQLVQSGAELKKPGASVKVSCKASGYTFTSYWINWIRQAPGQGLEWIGRIAPGSGTTYYNEVFKGRVTITVDKSTSTAYMELSSLRSEDTAVYFCARGDYGSYWGQGTLVTVSS

SEQ ID NO: 323 - protein, 107 aa, synthetic construct
EIVLTQSPATLSLSPGERATLSCSASSSISYMHWFQQRPGQSPRRWIYDTSKLASGVPARFSGSGSGTDYTLTISSLEPEDFATYYCHQRTSYPLTFGQGTKLEIKR

SEQ ID NO: 324 - protein, 448 aa, synthetic construct
EVQLVESGGGLVKPGGSLRLSCAASGFTFSSYSMNWVRQAPGKGLEWVSSISSSSSYIYYADSVKGRFTISRDNAKNSLYLQMNSLRAEDTAVYYCARVGLMGPFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 325 - protein, 214 aa, synthetic construct; identical to SEQ ID NO: 307

SEQ ID NO: 326 - protein, 443 aa, synthetic construct
EVQLLQSGPELEKPGASVMISCKASGSSFTGYNMNWVRQNIGKSLEWIGAIDPYYGGTSYNQKFKGRATLTVDKSSSTAYMHLKSLTSEDSAVYYCVSGMEYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 327 - protein, 220 aa, synthetic construct
EIVMTQSPATLSVSPGERATLSCRSSQSLVHRNGNTYLHWYLQKPGQSPKLLIHKVSNRFSGVPDRFSGSGSGTDFTLKISRVEAEDLGVYFCSQSTHVPPLTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 328 - protein, 576 aa, synthetic construct
EVQLVQSGAEVEKPGASVKISCKASGSSFTGYNMNWVRQNIGKSLEWIGAIDPYYGGTSYNQKFKGRATLTVDKSTSTAYMHLKSLRSEDTAVYYCVSGMEYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 329 - protein, 220 aa, synthetic construct
DVVMTQTPLSLPVTPGEPASISCRSSQSLVHRNGNTYLHWYLQKPGQSPKLLIHKVSNRFSGVPDRFSGSGSGTDFTLKISRVEAEDLGVYFCSQSTHVPPLTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 330 - protein, 449 aa, synthetic construct
QVQLVESGPGVVQPGRSLRISCAVSGFSVTNYGVHWVRQPPGKGLEWLGVIWAGGITNYNSAFMSRLTISKDNSKNTVYLQMNSLRAEDTAMYYCASRGGHYGYALDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 331 - protein, 211 aa, synthetic construct
EIVMTQTPATLSVSAGERVTITCKASQSVSNDVTWYQQKPGQAPRLLIYSASNRYSGVPARFSGSGYGTEFTFTISSVQSEDFAVYFCQQDYSSFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 332 - protein, 449 aa, synthetic construct
QVQLVESGPGVVQPGRSLRISCAVSGFSVTNYGVHWVRQPPGKGLEWLGVIWAGGITNYNSAFMSRLTISKDNSKNTVYLQMNSLRAEDTAMYYCASRGGHYGYALDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 333 - protein, 484 aa, synthetic construct; identical to SEQ ID NO: 177

SEQ ID NO: 334 - protein, 118 aa, synthetic construct
QVQLVQSGAEVKKPGASVKVSCKGSGYTFTDYAMYWVRQAPGQGLEWIGVIRTYSGDVTYNQKFKDRATMTVDKSISTAYMELSRLRSDDTAVYYCAKSGTVRGFAYWGQGTLVTVSS

SEQ ID NO: 335 - protein, 113 aa, synthetic construct
DIVMTQSPDSLAVSLGERATINCKSSQSLLNSGNQKNYLTWYQQKPGQPPKLLIYWASTRESGVPDRFSGSGSGTDFTLTISSLQAEDVAVYHCQNDYSYPYTFGQGTKLEIK

SEQ ID NO: 336 - protein, 447 aa, synthetic construct
QVQLVQSGAEVKKPGASVKVSCKGSGYTFTDYAMYWVRQAPGQGLEWIGVIRTYSGDVTYNQKFKDRATMTVDKSISTAYMELSRLRSDDTAVYYCAKSGTVRGFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 337 - protein, 220 aa, synthetic construct
DIVMTQSPDSLAVSLGERATINCKSSQSLLNSGNQKNYLTWYQQKPGQPPKLLIYWASTRESGVPDRFSGSGSGTDFTLTISSLQAEDVAVYHCQNDYSYPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 338 - protein, 449 aa, synthetic construct
QVTLRESGPALVKPTQTLTLTCTFSGFSLSTSGMGVGWIRQPPGKALEWLAHIWWDDDKYYNPSLKSRLTISKDTSKNQVVLTMTNMDPVDTATYYCARTRRYFPFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 339 - protein, 214 aa, synthetic construct
EIVMTQSPATLSVSPGERATLSCKASQNVGTNVAWYQQKPGQAPRLLIYSASYRYSGIPARFSGSGSGTEFTLTISSLQSEDFAVYYCQQYNTDPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 340 - protein, 594 aa, synthetic construct
EVQLLESGGGLVQPGGSLRLSCAASGFTFSSYAMSWVRQAPGKGLEWVSAIIGSGASTYYADSVKGRFTISRDNSKNTLYLQMNSLRAEDTAVYYCAKGWFGGFNYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSAPASSSTKKTQLQLEHLLLDLQMILNGINNYKNPKLTRMLTAKFAMPKKATELKHLQCLEEELKPLEEVLNGAQSKNFHLRPRDLISNINVIVLELKGSETTFMCEYADETATIVEFLNRWITFAQSIISTLT

SEQ ID NO: 341 - protein, 215 aa, synthetic construct
EIVLTQSPGTLSLSPGERATLSCRASQSVTSSYLAWYQQKPGQAPRLLINVGSRRATGIPDRFSGSGSGTDFTLTISRLEPEDFAVYYCQQGIMLPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 342 - protein, 447 aa, synthetic construct
EVQLLESGGGLVQPGGSLRLSCAASGFTFSSYAMSWVRQAPGKGLEWVSAIIGSGASTYYADSVKGRFTISRDNSKNTLYLQMNSLRAEDTAVYYCAKGWFGGFNYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 343 - protein, 215 aa, synthetic construct; identical to SEQ ID NO: 341

SEQ ID NO: 344 - protein, 579 aa, synthetic construct
EIQLVQSGAEVKKPGETVKISCKASGYTFTNYGMNWVKQTPGKGLKWMGWINTYTGEPTYADDFKGRFAFSLETSTSTAFLQINNLRSEDTATYFCVRFISKGDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 345 - protein, 213 aa, synthetic construct
EIVLTQSPATLSLSPGERVTLTCSASSSVSYMLWYQQKPGSSPKPWIFDTSNLASGFPARFSGSGSGTSYSLIISSMEAEDAATYYCHQRSGYPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 346 - protein, 449 aa, synthetic construct
QVQLVESGGGLVKPGGSLRLSCAVSGFTLSDYYMAWIRQAPGKGLEWVSTISASGLRTYYPDSVKGRFTISRDNAKNSLYLQMNSLRAEDTAVYYCARPLSAHYGFNYFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPGK

SEQ ID NO: 347 - protein, 214 aa, synthetic construct
DIQMTQSPSSLSASVGDRVTITCRTSEDIYQGLAWYQQKPGKAPKLLLYSANTLHIGVPSRFSGSGSGTDYTLTISSLQPEDFATYYCQQYYDYPLAFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 348 - protein, 450 aa, synthetic construct
EVQLVESGGGLVQPGRSLRLSCAASGFTFDDHAMHWVRQAPGKGLEWVSGISWNSRGIGYADSVKGRFTIFRDNAKNSLYLQMNSLRAEDTALYYCAKDEYSRGYYVLDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 349 - protein, 216 aa, synthetic construct
AIQLTQSPSSLSASVGDRVTITCRASQGISSALAWYQQKPGKAPKLLIYDASSLESGVPSRFSGSGSGTDFTLTISSLQPEDFATYYCQQFNSYPLWITFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 350 - protein, 432 aa, synthetic construct
EVQLVESGGGLVKPGGSLRLSCAASGFTFDDSVMHWVRQAPGKGLEWVSLVGWDGFFTYYADSVKGRFTISRDNAKNSLYLQMNSLRAEDTAVYYCARQGDYMGNNWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 351 - protein, 215 aa, synthetic construct
NFMLTQPHSVSESPGKTVTISCTRSSGSIDSSYVQWYQQRPGSSPTTVIYEDDQRPSGVPDRFSGSIDSSSNSASLTISGLKTEDEADYYCQSYDFHHLVFGGGTKLTVLQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 352 - protein, 454 aa, synthetic construct; identical to SEQ ID NO: 111

SEQ ID NO: 353 - protein, 215 aa, synthetic construct; identical to SEQ ID NO: 112

SEQ ID NO: 354 - protein, 122 aa, synthetic construct
QVTLVESGGGVVQPGRSLRLSCAFSGFSLSTSDMGVGWIRQAPGKGLEWVAHIWWDDVKRYNPALKSRFTISRDNSKNTLYLQMNSLRAEDTAVYFCARIEDYGVSYYFDYWGQGTTVTVSS

SEQ ID NO: 355 - protein, 107 aa, synthetic construct
DIQMTQSPSFLSASVGDRVSITCKASQDVSTAVAWYQQKPGKAPKLLIYSASYRYTGVPDRFSGSGSGTDFTLTISSLQPEDFATYYCQQHYSIPLTFGQGTKLEIK

SEQ ID NO: 356 - protein, 441 aa, synthetic construct
EVQLVQSGAEVKKPGATVKISCKASGFSIKDDYIHWVQQAPGKGLEWMGWIDAMNDDSQYSSKFQGRVTITVDTSTNTAYMKLSSLRSEDTAVYYCTYAFGGYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 357 - protein, 219 aa, synthetic construct
DIVMTQTPLSLSVTPGQPASISCRSSQSLVHSDSNTYLHWYLQKPGQSPQLLIYLVSNRFSGVPDRFSGSGSGTDFTLKISRVEAEDVGVYFCGQSTHVPYAFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 358 - protein, 446 aa, synthetic construct
QMQLVQSGPEVKKPGTSVKVSCKASGYTFTDYNVDWVRQARGQRLEWIGDINPNDGGTIYAQKFQERVTITVDKSTSTAYMELSSLRSEDTAVYYCARNYRWFGAMDHWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 359 - protein, 218 aa, synthetic construct
DIVMTQTPLSLSVTPGQPASISCKASQSLDYEGDSDMNWYLQKPGQPPQLLIYGASNLESGVPDRFSGSGSGTDFTLKISRVEAEDVGVYYCQQSTEDPRTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 360 - protein, 449 aa, synthetic construct
QVQLVESGGGVVQPGRSLRLSCVASGFTFSSYGMHWVRQAPGKGLEWVAIIWYDGSNKYYADSVKGRFTISRDNSKNTQYLQMNSLRAEDTAVYYCASVATSGDFDYYGMDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 361 - protein, 214 aa, synthetic construct
EIVLTQSPATLSLSPGERTTLSCRASQRISTYLAWYQQKPGQAPRLLIYDASKRATGIPARFSGSGSGTGFTLTISSLEPEDFAVYYCQQRSNWPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 362 - protein, 451 aa, synthetic construct
EVQLVESGGGLVQPGRSLRLSCAASGFTFDDYAMHWVRQTPGKGLEWVSGISWKSNIIGYADSVKGRFTISRDNAKNSLYLQMNSLRAEDTALYYCARDITGSGSYGWFDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSWDEPWGEDVSLTCLVKGFYPSDIAVEWESNGQPENNYKTTPPVLDSDGSFFLYSKLTVPYDRWVWPDEFSCSVMHEALHNHYTQKSLSLSPG

SEQ ID NO: 363 - protein, 214 aa, synthetic construct
DIQMTQSPSSLSASVGDRVTITCRASQSISSYLNWYQQKPGKAPKPLIYVASSLQSGVPSSFSGSGSGTDFTLTISSLQPEDFATYYCQQSYSNPITFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 364 - protein, 446 aa, synthetic construct
QVQLQESGPGLVKPSETLSLTCTVSGFSLTAYGVNWIRQPPGKGLEWIGMIWDDGSTDYDSALKSRVTISVDTSKNQFSLKLSSVTAADTAVYYCAREGDVAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 365 - protein, 220 aa, synthetic construct
DIQMTQSPSSLSASVGDRVTITCKSSQSLLNPSNQKNYLAWYQQKPGKAPKLLVYFASTRDSGVPSRFSGSGSGTDFTLTISSLQPEDFATYYCLQHFGTPPTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 366 - protein, 451 aa, synthetic construct
QVQLVQSGAEVKKPGASVKVSCKASGFTLTNYGMNWVRQARGQRLEWIGWINTDTGEPTYADDFKGRFVFSLDTSVSTAYLQISSLKAEDTAVYYCARNPPYYYGTNNAEAMDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 367 - protein, 214 aa, synthetic construct
DIQMTQSPSSLSASVGDRVTITCSSSQDISNYLNWYLQKPGQSPQLLIYYTSTLHLGVPSRFSGSGSGTEFTLTISSLQPDDFATYYCQQYYNLPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 368 - protein, 450 aa, synthetic construct
QVQMVQSGAEVKKPGASVKVSCKASGFNIKDTYIHWVRQAPGQGLEWMGEIDPANDNTKYDPKFQGRVTITADTSTSTVYMELSSLRSEDTAVYYCATYYYKYDVGGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 369 - protein, 215 aa, synthetic construct
EIVLTQSPGTLSLSPGERATLSCSVSSSISSSNLHWYQQKPGQAPRLLIYGTSNLASGIPDRFSGSGSGTDFTLTISRLEPEDFAVYYCQQWSSYPFTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 370 - protein, 119 aa, synthetic construct
QVQLVQSGAEVKKPGASVKVSCKASGYSFTSYWMNWVRQAPGQGLEWIGVIHPSDSETWLDQKFKDRVTITVDKSTSTAYMELSSLRSEDTAVYYCAREHYGTSPFAYWGQGTLVTVSS

SEQ ID NO: 371 - protein, 107 aa, synthetic construct
DIQMTQSPSSLSASVGDRVTITCRASQDVSSVVAWYQQKPGKAPKLLIYSASYRYTGVPSRFSGSGSGTDFTLTISSLQPEDFATYYCQQHYSTPWTFGGGTKLEIK

SEQ ID NO: 372 - protein, 448 aa, synthetic construct
QVTLVESGGGVVQPGRSLRLSCAFSGFSLSTSDMGVGWIRQAPGKGLEWVAHIWWDDVKRYNPALKSRFTISRDNSKNTLYLQMNSLRAEDTAVYFCARIEDYGVSYYFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 373 - protein, 214 aa, synthetic construct
DIQMTQSPSFLSASVGDRVSITCKASQDVSTAVAWYQQKPGKAPKLLIYSASYRYTGVPDRFSGSGSGTDFTLTISSLQPEDFATYYCQQHYSIPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 374 - protein, 119 aa, synthetic construct
QMQLVQSGPEVKKPGTSVKVSCKASGYTFTDYNVDWVRQARGQRLEWIGDINPNDGGTIYAQKFQERVTITVDKSTSTAYMELSSLRSEDTAVYYCARNYRWFGAMDHWGQGTTVTVSS

SEQ ID NO: 375 - protein, 111 aa, synthetic construct
DIVMTQTPLSLSVTPGQPASISCKASQSLDYEGDSDMNWYLQKPGQPPQLLIYGASNLESGVPDRFSGSGSGTDFTLKISRVEAEDVGVYYCQQSTEDPRTFGGGTKVEIK

SEQ ID NO: 376 - protein, 123 aa, synthetic construct
QVQLVESGGGVVQPGRSLRLSCVASGFTFSSYGMHWVRQAPGKGLEWVAIIWYDGSNKYYADSVKGRFTISRDNSKNTQYLQMNSLRAEDTAVYYCASVATSGDFDYYGMDVWGQGTTVTVSS

SEQ ID NO: 377 - protein, 90 aa, synthetic construct
EIVLTQSPATLSLSPGERTTLSCRASQRISTYLAWYQQKPGQAPRLLIYDASKRATGIPARFSGSGSGTGFTLTISSLEPEDFAVYYCQQ

SEQ ID NO: 378 - protein, 447 aa, synthetic construct
QVQLQQWGAGLLKPSETLSLTCAVYGGSFSDYYWNWIRQPPGKGLEWIGEINHRGSTNSNPSLKSRVTLSLDTSKNQFSLKLRSVTAADTAVYYCAFGYSDYEYNWFDPWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 379 - protein, 214 aa, synthetic construct
EIVLTQSPATLSLSPGERATLSCRASQSISSYLAWYQQKPGQAPRLLIYDASNRATGIPARFSGSGSGTDFTLTISSLEPEDFAVYYCQQRSNWPLTFGQGTNLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 380 - protein, 496 aa, synthetic construct
DIQMTQSPSSLSASVGDRVTITCRASQDVSSVVAWYQQKPGKAPKLLIYSASYRYTGVPSRFSGSGSGTDFTLTISSLQPEDFATYYCQQHYSTPWTFGGGTKLEIKGGGSGGGGQVQLVQSGAEVKKPGASVKVSCKASGYSFTSYWMNWVRQAPGQGLEWIGVIHPSDSETWLDQKFKDRVTITVDKSTSTAYMELSSLRSEDTAVYYCAREHYGTSPFAYWGQGTLVTVSSGGCGGGEVAACEKEVAALEKEVAALEKEVAALEKESKYGPPCPPCPAPEFLGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 381 - protein, 271 aa, synthetic construct
EIVLTQSPATLSLSPGERATLSCRASESVDNYGMSFMNWFQQKPGQPPKLLIHAASNQGSGVPSRFSGSGSGTDFTLTISSLEPEDFAVYFCQQSKEVPYTFGGGTKVEIKGGGSGGGGQVQLVQSGAEVKKPGASVKVSCKASGYTFTDYNMDWVRQAPGQGLEWMGDINPDNGVTIYNQKFEGRVTMTTDTSTSTAYMELRSLRSDDTAVYYCAREADYFYFDYWGQGTTLTVSSGGCGGGKVAACKEKVAALKEKVAALKEKVAALKE

SEQ ID NO: 382 - protein, 114 aa, synthetic construct
EVQLVQSGAEVKKPGATVKISCKASGFSIKDDYIHWVQQAPGKGLEWMGWIDAMNDDSQYSSKFQGRVTITVDTSTNTAYMKLSSLRSEDTAVYYCTYAFGGYWGQGTTVTVSS

SEQ ID NO: 383 - protein, 112 aa, synthetic construct
DIVMTQTPLSLSVTPGQPASISCRSSQSLVHSDSNTYLHWYLQKPGQSPQLLIYLVSNRFSGVPDRFSGSGSGTDFTLKISRVEAEDVGVYFCGQSTHVPYAFGGGTKVEIK

SEQ ID NO: 384 - protein, 118 aa, synthetic construct
EVQLVESGGGLVQPGGSLRLSCAASGFTFSSYSMNWVRQAPGKGLEWVSYISSSSSTIDYADSVKGRFTISRDNAKNSLYLQMNSLRDEDTAVYYCARESGWYLFDYWGQGTLVTVSS

SEQ ID NO: 385 - protein, 107 aa, synthetic construct
DIQMTQSPSSLSASVGDRVTITCRASQGISSWLAWYQQKPEKAPKSLIYAASSLQSGVPSRFSGSGSGTDFTLTISSLQPEDFATYYCQQYNSYPPTFGGGTKVEIK

SEQ ID NO: 386 - protein, 453 aa, synthetic construct
QVQLVESGGGVVQPGRSLRLSCAASGFTFSSYGMHWVRQAPGKGLEWVAVISYDGSNKYYADSVKGRFTISRDNSKNTLYLQMNSLRAEDTAVYYCARGRPWYSETGTSAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 387 - protein, 213 aa, synthetic construct
DIQMTQSPSSLSASVGDRVTITCQASQDISNYLNWYQQKPGKAPKLLIYDASNLETGVPSRFSGSGSGTDFTFTISSLQPEDIATYYCQQSDHYPTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 388 - protein, 444 aa, synthetic construct
EVQLVESGGGLVQPGGSLRLSCAASGFTFSSYSMNWVRQAPGKGLEWVSYISSSSSTIDYADSVKGRFTISRDNAKNSLYLQMNSLRDEDTAVYYCARESGWYLFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 389 - protein, 214 aa, synthetic construct
DIQMTQSPSSLSASVGDRVTITCRASQGISSWLAWYQQKPEKAPKSLIYAASSLQSGVPSRFSGSGSGTDFTLTISSLQPEDFATYYCQQYNSYPPTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 390 - protein, 450 aa, synthetic construct
EVQLVESGGGLVQPGGSLRLSCAASGFTFSRYGMSWVRQAPGKGLELVATINSNGGRTYYPDSVKGRFTISRDNAKNSLYLQMNSLRAEDTAVYYCAREGITTAYAMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 391 - protein, 218 aa, synthetic construct
DIVMTQSPDSLAVSLGERATINCKASQSVDYDGDSYMHWYQQKPGQPPKLLIYAASILESGVPDRFSGSGSGTDFTLTISSLQAEDVAVYYCQQSNEDPRTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 392 - protein, 447 aa, synthetic construct
QITLKESGPTLVKPKQTLTLTCTFSGFSLSTSGMGVGWIRQPPGKALEWLAVIYWDDHQLYSPSLKSRLTITKDTSKNQVVLTMTNMDPVDTATYYCAHRRGAFQHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 393 - protein, 214 aa, synthetic construct
EIVLTQSPGTLSLSPGERATLSCRASQSVSSSYLAWYQQKPGQAPRLLIYGASSRATGIPDRFSGSGSGTDFTLTISRLEPEDFAVYYCQQYDSSLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 394 - protein, 451 aa, synthetic construct
QVQLQESGPGLVKPSQTLSLTCAVYGGSFSSGYWNWIRKHPGKGLEYIGYISYNGITYHNPSLKSRITINRDTSKNQYSLQLNSVTPEDTAVYYCARYKYDYDGGHAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 395 - protein, 214 aa, synthetic construct
DIQMTQSPSSLSASVGDRVTITCRASQDISNYLNWYQQKPGKAPKLLIYYTSKLHSGVPSRFSGSGSGTDYTLTISSLQPEDFATYYCQQGSALPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 396 - protein, 448 aa, synthetic construct
QVTLKESGPALVKPTQTLTLTCSFSGFSLSTSGMGVGWIRQPPGKALEWIAHIWWDDDKYYNTALKTRLTISKDTSKNQVVLTMTNMDPVDTATYYCARIDWDGFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 397 - protein, 213 aa, synthetic construct
EIVLTQSPATLSLSPGERATLSCRASSSVSYMHWYQQKPGQAPRPWIYATSNRATGIPARFSGSGSGTDYTLTISSLEPEDFAVYYCQQWSSNPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 398 - protein, 447 aa, synthetic construct
EVQLVQSGAEVKKPGASVKVSCKASGYTFTDSYMSWVRQAPGQGLEWIGDMYPDNGDSSYNQKFRERVTITRDTSTSTAYLELSSLRSEDTAVYYCVLAPRWYFSVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 399 - protein, 214 aa, synthetic construct
DIQMTQSPSSLSASVGDRVTITCRASQDISNYLNWYQQKPGKAPKLLIYYTSRLRSGVPSRFSGSGSGTDFTLTISSLQPEDFATYYCQQGHTLPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 400 - protein, 441 aa, synthetic construct
QVQLVESGGGVVQPGRSLRLSCAASGFTFLRYAMHWVRQAPGKGLEWVAVISYDGRYKYYADSVKGRFTISRDNSKNTLYLQMNSLRAEDTAVYYCTTTTFDSWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 401 - protein, 220 aa, synthetic construct
DIVMTQTPLSLPVTPGEAASISCRSSQSLLDSEDGNTYLDWYLQKPGQSPQLLIYTLSHRASGVPDRFSGSGSGTDFTLEISRVEAEDVGVYYCMQRRDFPFTFGQGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 402 - protein, 440 aa, synthetic construct
QVQLVESGGGVVQPGRSLRLSCAASGFTFSSYGMHWVRQAPGKGLEWVAVIWYDGSNKYYADSVKGRFTISRDNSKNTLYLQMNSLRAEDTAVYYCASNGDHWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 403 - protein, 214 aa, synthetic construct
EIVMTQSPATLSVSPGERATLSCRASQSVSSNLAWYQQKPGQAPRLLIYGASTRATGIPARFSGSGSGTEFTLTISSLQSEDFAVYYCQQYNNWPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 404 - protein, 448 aa, synthetic construct
EIQLVQSGAEVKKPGSSVKVSCKASGYTFTHYGMNWVRQAPGQGLEWVGWVNTYTGEPTYADDFKGRLTFTLDTSTSTAYMELSSLRSEDTAVYYCTREGEGLGFGDWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 405 - protein, 219 aa, synthetic construct
DVVMTQSPLSLPVTPGEPASISCRSSQSIVHSHGDTYLEWYLQKPGQSPQLLIYKVSNRFSGVPDRFSGSGSGTDFTLKISRVEAEDVGVYYCFQGSHIPVTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 406 - protein, 713 aa, synthetic construct; identical to SEQ ID NO: 298

SEQ ID NO: 407 - protein, 214 aa, synthetic construct; identical to SEQ ID NO: 309

SEQ ID NO: 408 - protein, 443 aa, synthetic construct
EVQLVESGGGLVQPGGSLRLSCAASGFTFSSYMMSWVRQAPGKGLEWVATISGGGANTYYPDSVKGRFTISRDNAKNSLYLQMNSLRAEDTAVYYCARQLYYFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 409 - protein, 214 aa, synthetic construct
DIQMTQSPSSLSASVGDRVTITCLASQTIGTWLTWYQQKPGKAPKLLIYTATSLADGVPSRFSGSGSGTDFTLTISSLQPEDFATYYCQQVYSIPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 410 - protein, 444 aa, synthetic construct
EVQLLESGGVLVQPGGSLRLSCAASGFTFSNFGMTWVRQAPGKGLEWVSGISGGGRDTYFADSVKGRFTISRDNSKNTLYLQMNSLKGEDTAVYYCVKWGNIYFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 411 - protein, 214 aa, synthetic construct
DIQMTQSPSSLSASVGDSITITCRASLSINTFLNWYQQKPGKAPNLLIYAASSLHGGVPSRFSGSGSGTDFTLTIRTLQPEDFATYYCQQSSNTPFTFGPGTVVDFRRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 412 - protein, 450 aa, synthetic construct
QVQLVQSGAEVKKPGSSVKVSCKASGGTFSSYAISWVRQAPGQGLEWMGGIIPIFDTANYAQKFQGRVTITADESTSTAYMELSSLRSEDTAVYYCARPGLAAAYDTGSLDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 413 - protein, 214 aa, synthetic construct
EIVLTQSPATLSLSPGERATLSCRASQSVRSYLAWYQQKPGQAPRLLIYDASNRATGIPARFSGSGSGTDFTLTISSLEPEDFAVYYCQQRNYWPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 414 - protein, 443 aa, synthetic construct
EVQLLESGGGLVQPGGSLRLSCAASGFTFSSYDMSWVRQAPGKGLEWVSTISGGGSYTYYQDSVKGRFTISRDNSKNTLYLQMNSLRAEDTAVYYCASPYYAMDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 415 - protein, 214 aa, synthetic construct
DIQLTQSPSFLSAYVGDRVTITCKASQDVGTAVAWYQQKPGKAPKLLIYWASTLHTGVPSRFSGSGSGTEFTLTISSLQPEDFATYYCQHYSSYPWTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 416 - protein, 446 aa, synthetic construct
EVMLVESGGGLVQPGGSLRLSCTASGFTFSKSAMSWVRQAPGKGLEWVAYISGGGGDTYYSSSVKGRFTISRDNAKNSLYLQMNSLRAEDTAVYYCARHSNVNYYAMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 417 - protein, 218 aa, synthetic construct
EIVLTQSPATLSLSPGERATMSCRASENIDVSGISFMNWYQQKPGQAPKLLIYVASNQGSGIPARFSGSGSGTDFTLTISRLEPEDFAVYYCQQSKEVPWTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 418 - protein, 450 aa, synthetic construct
QVQLVQSGAEVKKPGSSVKVSCKASGGTFSSTAISWVRQAPGQGLEWMGGIWPSFGTASYAQKFQGRVTITADESTSTAYMELSSLRSEDTAVYYCARAEYSSTGIFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLLCLVKGFYPSDIAVEWESNGQPENNYKTTPPVLRSDGSFFLYSKLTVDKSRWQQGNVFSCSVMHEALHNHYTQKSLSLSPGK

SEQ ID NO: 419 - protein, 214 aa, synthetic construct
DIQMTQSPSSVSASVGDRVTITCRASQGISSWLAWYQQKPGKAPKLLISAASSLQSGVPSRFSGSGSGTDFTLTISSLQPEDFATYYCQQANHLPFTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 420 - protein, 445 aa, synthetic construct
EVQLVESGGGLVKPGGSLRLSCAASGFTFSSYGMSWVRQAPGKRLEWVATISGGGRDTYYSDSVKGRFTISRDNAKNNLYLQMNSLRAEDTAVYYCSRQYGTVWFFNWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 421 - protein, 218 aa, synthetic construct
EIVLTQSPATLSLSPGERATLSCRASESVDSYGNSFMHWYQQKPGQPPRLLIYAASNQGSGVPARFSGSGSGTDFTLTISSLEPEDFAMYFCQQSKEVPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 422 - protein, 440 aa, synthetic construct
QIQLVQSGSELKKPGASVKVSCKASGYTFTNFGMNWVRQAPGQGLKWMGWISGYTREPTYAADFKGRFVISLDTSVSTAYLQISSLKAEDTAVYYCARDVFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 423 - protein, 217 aa, synthetic construct
DIVLTQSPASLAVSPGQRATITCRASESVDNYGYSFMNWFQQKPGQPPKLLIYRASNLESGVPARFSGSGSRTDFTLTINPVEADDTANYYCQQSNADPTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 424 - protein, 446 aa, synthetic construct
QVQLVQSGAEVKKPGASVKVSCKASGYTFTNYWIHWVRQAPGQGLEWMGEIDPYDSYTNYNQKFKGRVTMTVDKSTSTVYMELSSLRSEDTAVYYCARPGFTYGGMDFWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 425 - protein, 220 aa, synthetic construct
DIQMTQSPSSLSASVGDRVTITCKSSQSLFNSGNQKNYLAWYQQKPGKVPKLLIYGASTRDSGVPYRFSGSGSGTDFTLTISSLQPEDVATYYCQNDHYYPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 426 - protein, 719 aa, synthetic construct
EVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSEVQLVESGGGLVQPGGSLRLSCAASGFAFSSYDMSWVRQAPGKGLDWVATISGGGRYTYYPDSVKGRFTISRDNSKNNLYLQMNSLRAEDTALYYCANRYGEAWFAYWGQGTLVTVSSGGGGSGGGGSGGGGSGGGGSDIQMTQSPSSMSASVGDRVTFTCRASQDINTYLSWFQQKPGKSPKTLIYRANRLVSGVPSRFSGSGSGQDYTLTISSLQPEDMATYYCLQYDEFPLTFGAGTKLELKR

SEQ ID NO: 427 - protein, 480 aa, synthetic construct
EIVLTQSPATLSASPGERVTLTCRASQSVGNDVAWYQQKPGQAPRLLINYASHRYTGVPDRFTGSGYGTEFTLTISSVQSEDFGVYYCQQDFSSPRTFGGGTKVEIKGKPGSGKPGSGKPGSGKPGSEVQLVESGGGLVKPGGSLRLSCVASGFTFSNYWMNWVRQAPGKGLEWVAEIRLYSNNYATHYAESVKGRFTISRDDSKSTLYLQMNNLKTEDTGVYYCTRYYGNYGGYFDVWGRGTLVTVSSEPKSSDKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 428 - protein, 583 aa, synthetic construct
EVQLVESGGSVVRPGGSLRLSCAASGFTVDDYSMSWVRQVPGKGLEWVSGINWNAGRTRYADAVKGRFTISRDSAKNSLYLQMNSLRAEDTALYYCAREFNNFESNWFDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSRKEMTKNQVSLTCLVKGFYPSDIAVEWESNGQPENNYKTTPPVLKSDGSFFLYSKLTVDKSRWQQGNVFSCSVMHEALHNHYTQKSLSLSPGQGQDRHMIEMRQLIDIVDQLKNYVNDLVPEFLPAPEDVETNCEWSAFSCFQKAQLKSANTGNNERIINVSIKKLKAKPPSTNAGRRQKHRLTCPSCDSYEKKPPKEFLERFKSLLQKMIHQHLSSRTHGSEDS

SEQ ID NO: 429 - protein, 214 aa, synthetic construct
SYELTQPPSVSVSPGQTARITCSGDALPKKYAYWYQQKPGQAPVLVISEDAKRPSGIPERFSGSSSGTMATLTISGAQVEDEADYYCYSTDASGNHRVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 430 - protein, 499 aa, synthetic construct; identical to SEQ ID NO: 300

SEQ ID NO: 431 - protein, 269 aa, synthetic construct; identical to SEQ ID NO: 301

SEQ ID NO: 432 - protein, 440 aa, synthetic construct
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 433 - protein, 214 aa, synthetic construct
EIVLTQSPATLSLSPGERATLSCRASQSVSSYLAWYQQKPGQAPRLLIYDASNRATGIPARFSGSGSGTDFTLTISSLEPEDFAVYYCQQSSNWPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 434 - protein, 442 aa, synthetic construct
QVQLVQSGAEVKKPGSSVKVSCKASGFTFTTYYISWVRQAPGQGLEYLGYINMGSGGTNYNEKFKGRVTITADKSTSTAYMELSSLRSEDTAVYYCAIIGYFDYWGQGTMVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 435 - protein, 219 aa, synthetic construct
DVVMTQSPLSLPVTLGQPASISCRSSQSLLDSDGGTYLYWFQQRPGQSPRRLIYLVSTLGSGVPDRFSGSGSGTDFTLKISRVEAEDVGVYYCMQLTHWPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 436 - protein, 447 aa, synthetic construct
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 437 - protein, 218 aa, synthetic construct
EIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 438 - protein, 448 aa, synthetic construct
EVQLVESGGGLVQPGGSLRLSCAASGFAFSSYDMSWVRQAPGKGLDWVATISGGGRYTYYPDSVKGRFTISRDNSKNNLYLQMNSLRAEDTALYYCANRYGEAWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 439 - protein, 214 aa, synthetic construct; identical to SEQ ID NO: 309

SEQ ID NO: 440 - protein, 449 aa, synthetic construct
QVQLVQSGAEVKKPGASVKVSCKVSGYSLSKYDMSWVRQAPGKGLEWMGIIYTSGYTDYAQKFQGRVTMTEDTSTDTAYMELSSLRSEDTAVYYCATGNPYYTNGFNSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 441 - protein, 217 aa, synthetic construct
DIQMTQSPSSLSASVGDRVTITCQASQSPNNLLAWYQQKPGKAPKLLIYGASDLPSGVPSRFSGSGSGTDFTLTISSLQPEDFATYYCQNNYYVGPVSYAFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 442 - protein, 447 aa, synthetic construct
QVQLVQSGSELKKPGASVKISCKASGYTFTNYGMNWVRQAPGQGLQWMGWINTDSGESTYAEEFKGRFVFSLDTSVNTAYLQITSLTAEDTGMYFCVRVGYDALDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 443 - protein, 213 aa, synthetic construct
EIVLTQSPSSLSASVGDRVTITCSARSSVSYMHWFQQKPGKAPKLWIYRTSNLASGVPSRFSGSGSGTSYCLTINSLQPEDFATYYCQQRSSFPLTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 444 - protein, 445 aa, synthetic construct
QVQLVQSGAEVKKPGASVKVSCKASGYTFPSYYMHWVRQAPGQGLEWMGIINPEGGSTAYAQKFQGRVTMTRDTSTSTVYMELSSLRSEDTAVYYCARGGTYYDYTYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 445 - protein, 214 aa, synthetic construct
DIQMTQSPSTLSASVGDRVTITCRASQSISSWLAWYQQKPGKAPKLLIYEASSLESGVPSRFSGSGSGTEFTLTISSLQPDDFATYYCQQYNSFPPTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 446 - protein, 459 aa, synthetic construct
QVQLVQSGGGLVQPGGSLRLSCAASGFTFSSYWMYWVRQVPGKGLEWVSAIDTGGGRTYYADSVKGRFAISRVNAKNTMYLQMNSLRAEDTAVYYCARDEGGGTGWGVLKDWPYGLDAWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 447 - protein, 214 aa, synthetic construct
QPVLTQPLSVSVALGQTARITCGGNNIGSKNVHWYQQKPGQAPVLVIYRDSNRPSGIPERFSGSNSGNTATLTISRAQAGDEADYYCQVWDSSTAVFGTGTKLTVLQRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 448 - protein, 445 aa, synthetic construct
EVQLVQSGGGLVQPGGSLKLSCAASGFTFSSYGMSWVRQAPGKGLDWVATISGGGRDTYYPDSVKGRFTISRDNSKNNLYLQMNSLRAEDTALYYCARQKGEAWFAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMITRTPEVTCVVVDVSQEDPEVQFNWYVDGVEVHNAKTKPREEQFNSTYRVVSVLTPLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHAHYTQKSLSLSLGK

SEQ ID NO: 449 - protein, 218 aa, synthetic construct
DIVLTQSPASLAVSPGQRATITCRASESVDNYGISFMNWFQQKPGQPPKLLIYAASNKGTGVPARFSGSGSGTDFTLNINPMEENDTAMYFCQQSKEVPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 450 - protein, 450 aa, synthetic construct
QVQLVQSGAEVKKPGSSVKVSCKASGGTFSSYAISWVRQAPGQGLEWMGLIIPSFDTAGYAQKFQGRVAITVDESTSTAYMELSSLRSEDTAVYYCARAEHSSTGTFDYWGQGTLVTVSSASTKGPSVFPEAPSSKSTSGGTAALGCLVTDYFPEPVTVSWNSGALTSGVHTFPAVLESSGLYSLSSVVTVPSSSLGTQTYICNVNHKPSNTKVDKRVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVLPPSRDELTKNQVSLLCLVKGFYPSDIAVEWESNGQPENNYLTWPPVLDSDGSFFLYSKLTVDKSRWQQGNVFSCSVMHEALHNHYTQKSLSLSPGK

SEQ ID NO: 451 - protein, 214 aa, synthetic construct
DIQMTQSPSSVSASVGDRVTITCRASQGISSWLAWYQQKPGKAPKLLISAASSLQSGVPSRFSGSGSGTDFTLTISSLQPEDFATYYCQQANHLPFTFGGGTKVEIKRTVAAPSVFIFPPSDEQLKSGTARVGCLLNNFYPREAKVQWKVDNALQSGNSQESVTEQDSKDSTYSLRSTLTLSKADYEKHKVYACEVTHQGLSSPVTKSFNRGEC

SEQ ID NO: 452 - protein, 445 aa, synthetic construct
QVQLVQSGAEVKKPGASVKVSCKASGYSFTSYWMNWVRQAPGQGLEWIGVIHPSDSETWLDQKFKDRVTITVDKSTSTAYMELSSLRSEDTAVYYCAREHYGTSPFAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 453 - protein, 218 aa, synthetic construct
EIVLTQSPATLSLSPGERATLSCRASESVDNYGMSFMNWFQQKPGQPPKLLIHAASNQGSGVPSRFSGSGSGTDFTLTISSLEPEDFAVYFCQQSKEVPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 454 - protein, 451 aa, synthetic construct
QVQLVQSGSELKKPGASVKVSCKASNYTFTDYSMHWVRQAPGQGLEWMGWINIETYYPTYADQFKGRFAFSLDTSVSTAYLQISSLKAEDTAVYYCARDYYGRFYYAMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 455 - protein, 215 aa, synthetic construct
EIVLTQSPATLSLSPGERATLSCTASSSVSSSYFHWYQQKPGQAPRLLIYSTSNLASGIPARFSGSGSGTDFTLTISRLEPEDFAVYYCHQYHRSPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 456 - protein, 448 aa, synthetic construct
EVQLVESGGGLVKPGGSLRLSCAASGFTFSSYGMSWVRQTPEKRLEWVATISGGGRDTYYPDSVKGRFTISRDNAKNNLYLQMSSLRSEDTALYYCARQKDTSWFVHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDQLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHNHYTQKSLSLSPGK

SEQ ID NO: 457 - protein, 218 aa, synthetic construct
EIVLTQSPATLAVSPGERATISCRASESVDDYGISFMNWFQQKPGQPPKLLIYVASNQGSGVPARFSGSGSGTDFTLNIHPMEEDDTAMYFCQQSKEVPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 458 - protein, 444 aa, synthetic construct
QVQLVQSGAEVKKPGASVKVSCKASGYTFTSYWINWVRQAPGQGLEWMGNIYPGSSLTNYNEKFKNRVTMTRDTSTSTVYMELSSLRSEDTAVYYCARLSTGTFAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 459 - protein, 220 aa, synthetic construct
DIVMTQSPDSLAVSLGERATINCKSSQSLWDSGNQKNFLTWYQQKPGQPPKLLIYWTSYRESGVPDRFSGSGSGTDFTLTISSLQAEDVAVYYCQNDYFYPHTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 460 - protein, 443 aa, synthetic construct
QVQLVESGGGLVKPGGSLRLSCAASGFTFSNYGMSWIRQAPGKGLEWVSTISGGGSNIYYADSVKGRFTISRDNAKNSLYLQMNSLRAEDTAVYYCVSYYYGIDFWGQGTSVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 461 - protein, 214 aa, synthetic construct
DIQMTQSPSSLSASVGDRVTITCKASQDVTTAVAWYQQKPGKAPKLLIYWASTRHTGVPSRFSGSGSGTDFTLTISSLQPEDFATYYCQQHYTIPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 462 - protein, 447 aa, synthetic construct
QVQLVQSGAEVKKPGSSVKVSCKASGGTFSSYAISWVRQAPGQGLEWMGLIIPMFDTAGYAQKFQGRVAITVDESTSTAYMELSSLRSEDTAVYYCARAEHSSTGTFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 463 - protein, 214 aa, synthetic construct; identical to SEQ ID NO: 419

SEQ ID NO: 464 - protein, 443 aa, synthetic construct
EVQLVQSGAEVKKPGESLRISCKGSGYTFTTYWMHWVRQATGQGLEWMGNIYPGTGGSNFDEKFKNRVTITADKSTSTAYMELSSLRSEDTAVYYCTRWTTGTGAYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 465 - protein, 220 aa, synthetic construct
EIVLTQSPATLSLSPGERATLSCKSSQSLLDSGNQKNFLTWYQQKPGQAPRLLIYWASTRESGVPSRFSGSGSGTDFTFTISSLEAEDAATYYCQNDYSYPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 466 - protein, 447 aa, synthetic construct
EVQLVESGGGLVQPGGSLRLSCAASGFTFSETWLHWVRQAPGKGLEWVAWVSPFGGSTYYADSVKGRFTISADTSKNTAYLQMNSLRAEDTAVYYCARRHWPGGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 467 - protein, 214 aa, synthetic construct
DIQMTQSPSSLSASVGDRVTITCRASQDVSTAVAWYQQKPGKAPKLLIYSASFLYSGVPSRFSGSGSGTDFTLTISSLQPEDFATYYCQQFLYHPATFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 468 - protein, 445 aa, synthetic construct
QVQLQESGPGLVKPSETLSLTCTVSGFSLTSYGVHWIRQPPGKGLEWIGVIYADGSTNYNPSLKSRVTISKDTSKNQVSLKLSSVTAADTAVYYCARAYGNYWYIDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPPVAGGPSVFLFPPKPKDTLMISRTPEVTCVVVAVSQEDPEVQFNWYVDGVEVHNAKTKPREEQFNSTYRVVSVLTVVHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSKLTVDKSRWQEGNVFSCSVMHEALHNHYTQKSLSLSLGK

SEQ ID NO: 469 - protein, 214 aa, synthetic construct
DIVMTQSPDSLAVSLGERATINCKSSESVSNDVAWYQQKPGQPPKLLINYAFHRFTGVPDRFSGSGYGTDFTLTISSLQAEDVAVYYCHQAYSSPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 470 - protein, 452 aa, synthetic construct
QGQLVQSGAEVKKPGASVKVSCKASGYTFTDYEMHWVRQAPIHGLEWIGVIESETGGTAYNQKFKGRVTITADKSTSTAYMELSSLRSEDTAVYYCAREGITTVATTYYWYFDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 471 - protein, 219 aa, synthetic construct
DVVMTQSPLSLPVTLGQPASISCRSSQSIVHSNGNTYLEWYLQKPGQSPQLLIYKVSNRFSGVPDRFSGSGSGTDFTLKISRVEAEDVGVYYCFQGSHVPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 472 - protein, 447 aa, synthetic construct; identical to SEQ ID NO: 302

SEQ ID NO: 473 - protein, 215 aa, synthetic construct; identical to SEQ ID NO: 303

SEQ ID NO: 474 - protein, 450 aa, synthetic construct
EVQLLESGGGLVQPGGSLRLSCAASGFTFSSYDMSWVRQAPGKGLEWVSLISGGGSQTYYAESVKGRFTISRDNSKNTLYLQMNSLRAEDTAVYFCASPSGHYFYAMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 475 - protein, 214 aa, synthetic construct
DIQMTQSPSSVSASVGDRVTITCRASQGISNWLAWYQQKPGKAPKLLIFAASSLQSGVPSRFSGSGSGTDFTLTISSLQPEDFATYYCQQAESFPHTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 476 - protein, 450 aa, synthetic construct
QLQLQESGPGLVKPSETLTLTCTVSADSISSTTYYWVWIRQPPGKGLEWIGSISYSGSTYYNPSLKSRVTVSVDTSKNQFSLKLNSVAATDTALYYCARHLGYNGRYLPFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 477 - protein, 216 aa, synthetic construct
QSALTQPASVSGSPGQSITISCTGTSSDVGFYNYVSWYQQHPGKAPELMIYDVSNRPSGVSDRFSGSKSGNTASLTISGLQAEDEADYYCSSYTSISTWVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 478 - protein, 496 aa, synthetic construct; identical to SEQ ID NO: 380

SEQ ID NO: 479 - protein, 271 aa, synthetic construct; identical to SEQ ID NO: 381

SEQ ID NO: 480 - protein, 123 aa, synthetic construct
EIQLQQSGVELVRPGASVTLSCKASGYTFTDYDMHWVKQTPVHGLEWIGTIDPETGGTAYNQKFKGKATLTADRSSTTAYMELSSLTSEDSAVYYCTSFYYTYSNYDVGFAYWGQGTLVTVSA

SEQ ID NO: 481 - protein, 112 aa, synthetic construct
DIVMTQAAPSVPVTPGESVSISCRSTKSLLHSNGNTYLYWFLQRPGQSPQLLIYRMSNLASGVPDRFSGSGSGTAFTLRISRVEAEDVGVYYCMQHLEYPFTFGGGTKLEIK

SEQ ID NO: 482 - protein, 115 aa, synthetic construct
QLQLQESGPGLVKPSETLSLTCAVSGASISNWWSWVRQPPGKGLEWIGEVHHSGVTTYKPSLKSRVTISVDNSKNQLSKLTSVTAADTAVYYCAREFADDAFDIWGRGTMVTVSS

SEQ ID NO: 483 - protein, 109 aa, synthetic construct
SSELTQDPAVSVALGQTVRITCRGDSLRKYYASWYQQKPRQAPQLVIYHKNNRASGIPDRFSGSISGNTASLTITGAQAEDEAAYFCNSRDTSGNYLVFGGGTKVTVLG

SEQ ID NO: 484 - protein, 456 aa, synthetic construct
EVQLQQSGPGLVKPSQTLSLTCAISGDSVSSNSAAWNWIRQSPSRGLEWLGKTYYRFKWYSDYAVSVKGRITINPDTSKNQFSLQLNSVTPEDTAVFYCTRESTTYDLLAGPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 485 - protein, 220 aa, synthetic construct
DIVMTQSPDSLAVSLGERATINCKSSQTVLYSSNNKKYLAWYQQKPGQPPNLLIYWASTRESGVPDRFSGSGSGTDFTLTISSLQAEDVAVYYCQQYYSTPFTFGPGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 486 - protein, 448 aa, synthetic construct
QVQLQESGPGLVKPSETLSLTCAVSGYSITSDYAWNWIRQPPGKGLEWIGYISYSGSTSYNPSLRSRVTISRDTSKNQFFLKLSSVTAADTAVYYCARRQVGLGFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 487 - protein, 214 aa, synthetic construct
DIQMTQSPSSLSASVGDRVTITCKASQDVSTAVAWYQQKPGKAPKLLIYSASYRYTGVPSRFSGSGSGTDFTFTISSLQPEDIATYYCQQHYSTPWTFGQGTKVEIKRTVAAPSVFIFPPSDEQLKSGTASVVCLLNNFYPREAKVQWKVDNALQSGNSQESVTEQDSKDSTYSLSNTLTLSKADYEKHKVYACEVTHQGLSSPVTKSFNRGEC

SEQ ID NO: 488 - protein, 449 aa, synthetic construct
EVQLVQSGAEVKKPGSSVKVSCKASGYTFSSYVMHWVRQAPGQGLEWIGYIDPYNDGAKYAQKFQGRVTLTSDKSTSTAYMELSSLRSEDTAVYYCARGGPYGWYFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 489 - protein, 214 aa, synthetic construct
DIQMTQSPSSLSASVGDRVTITCRASEHIYSYLSWYQQKPGKVPKLLIYNAKTLAEGVPSRFSGSGSGTDFTLTISSLQPEDVATYYCQHHFGSPLTFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 490 - protein, 448 aa, synthetic construct
EVQLVESGGGLVQPGGSLRLSCAASGFTFSNFGMHWVRQAPGKGLEWVAFISSGSSSIYYADTVKGRFTISRDNAKNSLYLQMNSLRAEDTAVYYCARMRLDYYAMDYWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 491 - protein, 214 aa, synthetic construct
DIQMTQSPSSLSASVGDRVTITCRASKSISKYLAWYQQKPGKAPKLLIYSGSTLQSGVPSRFSGSGSGTDFTLTISSLQPEDFATYYCQQHNEYPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 492 - protein, 449 aa, synthetic construct
EVQLVESGGGLVQPGGSLRLSCAASGFTFSDYYMYWVRQAPGKGLEWVAYITKGGGSTYYPDSVKGRFTISRDNAKNTLYLQMNSLRAEDTAVYYCARQTNYDFTMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 493 - protein, 214 aa, synthetic construct
EIVMTQSPATLSVSPGERATLSCKASQDVGTSVAWYQQKPGQAPRLLIYWASARHTGIPARFSGSGSGTEFTLTISSLQSEDFAVYYCQQYSSYPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 494 - protein, 451 aa, synthetic construct
QVQLVQSGAEVKKPGASVKVSCKASGYTFTSYYMHWVRQAPGQGLEWMGVIGPSGASTSYAQKFQGRVTLTRDTSTSTVYMELSSLRSEDTAVYYCARDHSDYWSGIMEVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 495 - protein, 215 aa, synthetic construct
EIVLTQSPGTLSLSPGERATLSCRASQSVRSSYLAWYQQKPGQAPRLLIYGASSRATGIPDRFSGSGSGTDFTLTISRLEPEDFAVYYCQQYFSPPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 496 - protein, 451 aa, synthetic construct
QVQLVQSGSELKKPGASVKVSCKASGYTFTSYPMNWVRQAPGQGLEWMGWINTNTGNPTYAQGFTGRFVFSLDTSVSTAYLQISSLKAEDTAVYYCARVGGYSVDEYAFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 497 - protein, 213 aa, synthetic construct
DIQLTQSPSFLSASVGDRVTITCRASQGISSYLAWYQQKPGKAPKLLIYAASTLQSGVPSRFSGSGSGTEFTLTISSLQPEDFATYYCQQLSSYPTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 498 - protein, 451 aa, synthetic construct
QVQLQESGPGLVKPSGTLSLTCAVSGYSITSGYSWHWVRQPPGKGLEWIGYVHYSGSTNYNPSLKSRVTISVDKSKNQFSLKLSSVTAADTAVYYCARMDYGNYGGAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 499 - protein, 214 aa, synthetic construct
EIVMTQSPATLSVSPGERATLSCKASQDVRTAVAWYQQKPGQAPRLLIYSASYRYTGIPARFSGSGSGTEFTLTISSLQSEDFAVYYCQQYYSTQWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 500 - protein, 440 aa, synthetic construct
EVQLLESGGGLVQPGGSLRLSCAAASGFTFSSYDMSWVRQAPGKGLDWVSTISGGGTYTYYQDSVKGRFTISRDNSKNTLYLQMNSLRAEDTAVYYCASMDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 501 - protein, 214 aa, synthetic construct
DIQMTQSPSSLSASVGDRVTITCRASQSIRRYLNWYHQKPGKAPKLLIYGASTLQSGVPSRFSGSGSGTDFTLTISSLQPEDFAVYYCQQSHSAPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 502 - protein, 444 aa, synthetic construct
QVQLVQSGAEVKKPGSSVKVSCKASGYTFTSYNMHWVRQAPGQGLEWMGDIYPGNGDTSYNQKFKGRVTITADKSTSTVYMELSSLRSEDTAVYYCARVGGAFPMDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 503 - protein, 218 aa, synthetic construct
AIQLTQSPSSLSASVGDRVTITCRASESVEYYGTSLMQWYQQKPGKAPKLLIYAASNVESGVPSRFSGSGSGTDFTLTISSLQPEDFATYFCQQSRKDPSTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 504 - protein, 448 aa, synthetic construct
EVQLVESGGGLVQPGGSLRLSCAASGFTFSRYAMSWVRQAPGKGLEWVAAISSGGSLYYPDSVKGRFTISRDNAKNTLYLQMNSLRAEDTAVYYCARGREADGGYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPAAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 505 - protein, 218 aa, synthetic construct
EIVLTQSPATLSVSPGERATLSCRASESVEYYGTSLMQWYQQKPGQAPRLLIYAASNVESGIPARFSGSGSGTEFTLTISSLQSEDFAVYYCQQSLKVPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 506 - protein, 112 aa, synthetic construct
QVQLQQWGAGLLKPSETLSLTCAVYGGSFSGYYWSWIRQPPGKGLEWIGEINHSGSTNYNPSLKSRVTISVDTSKNQFSLKLSSVTAADTAVYYCARVINWFDPWGQGTLVT

SEQ ID NO: 507 - protein, 107 aa, synthetic construct [sequence omitted]

SEQ ID NO: 508 - protein, 118 aa, synthetic construct
EIQLVQSGGGLVKPGGSVRISCAASGYTFTNYGMNWVRQAPGKGLEWMGWINTHTGEPTYADSFKGRFTFSLDDSKNTAYLQINSLRAEDTAVYFCTRRGYDWYFDVWGQGTTVTVSS

SEQ ID NO: 509 - protein, 107 aa, synthetic construct
DIQMTQSPSSLSASVGDRVTITCRASQDINSYLSWFQQKPGKAPKTLIYRANRLESGVPSRFSGSGSGTDYTLTISSLQYEDFGIYYCQQYDESPWTFGGGTKLEIK

SEQ ID NO: 510 - protein, 124 aa, synthetic construct
QVQLVQSGAEVKKPGASVKVSCKTSRYTFTEYTIHWVRQAPGQRLEWIGGINPNNGIPNYNQKFKGRVTITVDTSASTAYMELSSLRSEDTAVYYCARRRIAYGYDEGHAMDYWGQGTLVTVSS

SEQ ID NO: 511 - protein, 113 aa, synthetic construct
DIVMTQSPDSLAVSLGERATINCKSSQSLLYSRNQKNYLAWYQQKPGQPPKLLIFWASTRESGVPDRFSGSGFGTDFTLTISSLQAEDVAVYYCQQYFSYPLTFGQGTKVEIK

SEQ ID NO: 512-519 - skipped (8 entries)

SEQ ID NO: 520 - protein, 444 aa, synthetic construct
QVQLQESGPGLVKPSQTLSLTCTVSGGSISSGEYYWNWIRQHPGKGLEWIGYIYYSGSTYYNPSLKSRVTISVDTSKNQFSLKLSSVTAADTAVYYCARESVAGFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 521 - protein, 215 aa, synthetic construct
EIVLTQSPGTLSLSPGERATLSCRASQSVSSSYLAWYQQKPGQAPRLLIYGTSSRATGIPDRFSGSGSGTDFTLTISRLEPEDFAVYYCQQYGSSPITFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 522 - protein, 448 aa, synthetic construct
QVQLQQSGAEVKKPGSSVKVSCKASGYAFTSYVIHWVRQAPGQGLYWIGYIHPYNGGTKYNEKFKGKATITADESTNTAYMELSSLRSEDTAFYFCARSGGGDPFAYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 523 - protein, 213 aa, synthetic construct
DIQLTQSPSSLSASVGDRVTITCKASQDINKYIGWYQQKPGKAPKLLMHYTSALLPGIPSRFSGSGSGRDYTFTISSLQPEDIATYYCLQYDDLWTFGGGTKLQIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 524 - protein, 445 aa, synthetic construct
EVQLLESGGGLVQPGGSLRLSCAASGFTFSSYAMNWVRQAPGKGLEWVSTTSGSGASTYYADSVKGRFTISRDNSKNTLYLQMNSLRAEDTAVYYCAKIWIAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 525 - protein, 215 aa, synthetic construct
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 526 - protein, 446 aa, synthetic construct
EVQLVESGGGLVQPGGSLRLSCAASGYSITNDYAWNWVRQAPGKGLEWVGYISYSGYTTYNPSLKSRFTISRDTSKNTLYLQMNSLRAEDTAVYYCARWTSGLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 527 - protein, 214 aa, synthetic construct
DIQMTQSPSSLSASVGDRVTITCKASDLIHNWLAWYQQKPGKAPKLLIYGATSLETGVPSRFSGSGSGTDFTLTISSLQPEDFATYYCQQYWTTPFTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 528 - protein, 449 aa, synthetic construct
QVQLVQSGAEVKKPGETVKISCKASDYTFTYYGMNWVKQAPGQGLKWMGWIDTTTGEPTYAQKFQGRIAFSLETSASTAYLQIKSLKSEDTATYFCARRGPYNWYFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 529 - protein, 219 aa, synthetic construct
DIVMTQSPLSVPVTPGEPVSISCRSSKSLLHSNGNTYLYWFLQRPGQSPQLLIYRMSNLVSGVPDRFSGSGSGTAFTLRISRVEAEDVGVYYCLQHLEYPFTFGPGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 530 - protein, 449 aa, synthetic construct
DVKLVESGGGLVKLGGSLKLSCAASGFTFSNYYMSWVRQTPEKRLELVAAINSDGGITYYLDTVKGRFTISRDNAKNTLYLQMSSLKSEDTALFYCARHRSGYFSMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 531 - protein, 214 aa, synthetic construct
DIVMTQSQRFMSTTVGDRVSITCKASQNVVSAVAWYQQKPGQSPKLLIYSASNRYTGVPDRFTGSGSGTDFTLTISNMQSEDLADFFCQQYSNYPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 532 - protein, 442 aa, synthetic construct
EVKLVESGGGLVQPGGSLRLSCATSGFTFTDYYMNWVRQPPGKALEWLGFIGNKANGYTTEYSASVKGRFTISRDKSQSILYLQMNTLRAEDSATYYCTRDRGLRFYFDYWGQGTTLTVSSAKTTPPSVYPLAPGSAAQTNSMVTLGCLVKGYFPEPVTVTWNSGSLSSGVHTFPAVLQSDLYTLSSSVTVPSSPRPSETVTCNVAHPASSTKVDKKIVPRDCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSR

SEQ ID NO: 533 - protein, 213 aa, synthetic construct
QTVLSQSPAILSASPGEKVTMTCRASSSVTYIHWYQQKPGSSPKSWIYATSNLASGVPARFSGSGSGTSYSLTISRVEAEDAATYYCQHWSSKPPTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 534 - protein, 693 aa, synthetic construct; identical to SEQ ID NO: 138

SEQ ID NO: 535 - protein, 451 aa, synthetic construct
QVQLVQSGAEVKKPGASVKVSCKASGYTFTEFGMNWVRQAPGQGLEWMGWINTKTGEATYVEEFKGRVTFTTDTSTSTAYMELRSLRSDDTAVYYCARWDFAYYVEAMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 536 - protein, 215 aa, synthetic construct
DIQMTQSPSSLSASVGDRVTITCKASAAVGTYVAWYQQKPGKAPKLLIYSASYRKRGVPSRFSGSGSGTDFTLTISSLQPEDFATYYCHQYYTYPLFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 537 - protein, 449 aa, synthetic construct
EVQLVESGGDLVQPGRSLRLSCAASGFIFSNYGMSWVRQAPGKGLEWVATISSASTYSYYPDSVKGRFTISRDNAKNSLYLQMNSLRVEDTALYYCGRHSDGNFAFGYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 538 - protein, 219 aa, synthetic construct
DVLMTQSPLSLPVTPGEPASISCRSSRNIVHINGDTYLEWYLQKPGQSPQLLIYKVSNRFSGVPDRFSGSGSGTDFTLKISRVEAEDVGVYYCFQGSLLPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 539 - protein, 449 aa, synthetic construct
EVQLVESGGGLVKPGGSLRLSCAASGYTFSNYWIGWVRQAPGKGLEWIGDIYPGGNYIRNNEKFKDKTTLSADTSKNTAYLQMNSLKTEDTAVYYCGSSFGSNYVFAWFTYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 540 - protein, 219 aa, synthetic construct
DIVMTQSPLSLPVTPGEPASISCRSSQRLLSSYGHTYLHWYLQKPGQSPQLLIYEVSNRFSGVPDRFSGSGSGTDFTLKISRVEAEDVGVYYCSQSTHVPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 541 - protein, 504 aa, synthetic construct; identical to SEQ ID NO: 135

SEQ ID NO: 542 - protein, 450 aa, synthetic construct
QVQLVQPGAEVVKPGASVKLSCKTSGYTFTSNWMHWVKQAPGQGLEWIGEIDPSDSYTNYNQNFQGKAKLTVDKSTSTAYMEVSSLRSDDTAVYYCARGSNPYYYAMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 543 - protein, 211 aa, synthetic construct
EIVLTQSPAIMSASPGERVTMTCSASSGVNYMHWYQQKPGTSPRRWIYDTSKLASGVPARFSGSGSGTDYSLTISSMEPEDAATYYCHQRGSYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 544 - protein, 450 aa, synthetic construct
QVQLQESGPGLVKPSQTLSLTCTVSGGSISTSGMGVGWIRQHPGKGLEWIGHIWWDDDKRYNPALKSRVTISVDTSKNQFSLKLSSVTAADTAVYYCARMELWSYYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 545 - protein, 213 aa, synthetic construct
EIVLTQSPATLSLSPGERATLSCSASSSVSYMHWYQQKPGQAPRLLIYDTSKLASGIPARFSGSGSGTDFTLTISSLEPEDVAVYYCFQGSVYPFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 546 - protein, 502 aa, synthetic construct; identical to SEQ ID NO: 142

SEQ ID NO: 547 - protein, 271 aa, synthetic construct
QAVVTQEPSLTVSPGGTVTLTCRSSTGAVTTSNYANWVQQKPGQAPRGLIGGTNKRAPWTPARFSGSLLGGKAALTITGAQAEDEADYYCALWYSNLWVFGGGTKLTVLGGGGSGGGGQVTLRESGPALVKPTQTLTLTCTFSGFSLSTSGMGVGWIRQPPGKALEWLAHIWWDDDKRYNPALKSRLTISKDTSKNQVFLTMTNMDPVDTATYYCARMELWSYYFDYWGQGTTVTVSSASTKGKVAACKEKVAALKEKVAALKEKVAALKE

SEQ ID NO: 548 - protein, 451 aa, synthetic construct
EVQLVESGGGLVQPGGSLRLSCAASGFTFSSSWMNWVRQAPGKGLEWVGRIYPGDGDTNYNVKFKGRFTISRDDSKNSLYLQMNSLKTEDTAVYYCARSGFITTVRDFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 549 - protein, 218 aa, synthetic construct
EIVLTQSPDFQSVTPKEKVTITCRASESVDTFGISFMNWFQQKPDQSPKLLIHEASNQGSGVPSRFSGSGSGTDFTLTINSLEAEDAATYYCQQSKEVPFTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 550 - protein, 449 aa, synthetic construct
QVQLVQPGAEVVKPGASVKLSCKTSGYTFTSNWMHWVKQAPGQGLEWIGEIDPSDSYTNYNQNFQGKAKLTVDKSTSTAYMEVSSLRSDDTAVYYCARGSNPYYYAMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 551 - protein, 211 aa, synthetic construct
EIVLTQSPAIMSASPGERVTMTCSASSGVNYMHWYQQKPGTSPRRWIYDTSKLASGVPARFSGSGSGTSYSLTISSMEPEDAATYYCHQRGSYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 552 - protein, 451 aa, synthetic construct
EVQLVESGGGLVKPGGSLKLSCAASGYTFTSYVMHWVRQAPGKGLEWIGYINPYNDGTKYNEKFQGRVTISSDKSISTAYMELSSLRSEDTAMYYCARGTYYYGTRVFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPDVFLFPPKPKDTLMISRTPEVTCVVVDVSHEDPEVQFNWYVDGVEVHNAKTKPREEQFNSTFRVVSVLTVVHQDWLNGKEYKCKVSNKALPAPEEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 553 - protein, 219 aa, synthetic construct
DIVMTQSPATLSLSPGERATLSCRSSKSLQNVNGNTYLYWFQQKPGQSPQLLIYRMSNLNSGVPDRFSGSGSGTEFTLTISSLEPEDFAVYYCMQHLEYPITFGAGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 554 - protein, 449 aa, synthetic construct
QVQLVQSGAEVKKPGSSVKVSCKASGYAFSYSWINWVRQAPGQGLEWMGRIFPGDGDTDYNGKFKGRVTITADKSTSTAYMELSSLRSEDTAVYYCARNVFDGYWLVYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 555 - protein, 219 aa, synthetic construct
DIVMTQTPLSLPVTPGEPASISCRSSKSLLHSNGITYLYWYLQKPGQSPQLLIYQMSNLVSGVPDRFSGSGSGTDFTLKISRVEAEDVGVYYCAQNLELPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 556 - protein, 450 aa, synthetic construct
EVQLVQSGAEVKKPGESLKISCKGSGRTFTSYNMHWVRQMPGKGLEWMGAIYPLTGDTSYNQKSKLQVTISADKSISTAYLQWSSLKASDTAMYYCARSTYVGGDWQFDVWGKGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKIKDTLMISRTPEVTCVVVDVSHEDPEVKFNWYVDGVEVHNAKTKPREEQYNSTYRVVSVLTVLHQDWLNGKEYKCKVSNKALPAPIEKTISKQKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 557 - protein, 213 aa, synthetic construct
EIVLTQSPGTLSLSPGERATLSCRASSSVPYIHWYQQKPGQAPRLLIYATSALASGIPDRFSGSGSGTDFTLTISRLEPEDFAVYYCQQWLSNPPTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 558 - protein, 452 aa, synthetic construct
EVQLVESGGGLVQPGGSLRLSCAASGYTFTSYNMHWVRQAPGKGLEWVGAIYPGNGDTSYNQKFKGRFTISVDKSKNTLYLQMNSLRAEDTAVYYCARVVYYSNSYWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 559 - protein, 213 aa, synthetic construct
DIQMTQSPSSLSASVGDRVTITCRASSSVSYMHWYQQKPGKAPKPLIYAPSNLASGVPSRFSGSGSGTDFTLTISSLQPEDFATYYCQQWSFNPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 560 - protein, 452 aa, synthetic construct
EVQLVESGGGLVQPGRSLRLSCAASGFTFNDYAMHWVRQAPGKGLEWVSTISWNSGSIGYADSVKGRFTISRDNAKKSLYLQMNSLRAEDTALYYCAKDIQYGNYYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 561 - protein, 214 aa, synthetic construct; identical to SEQ ID NO: 152

SEQ ID NO: 562 - protein, 451 aa, synthetic construct
QVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 563 - protein, 213 aa, synthetic construct
QIVLSQSPAILSASPGEKVTMTCRASSSVSYIHWFQQKPGSSPKPWIYATSNLASGVPVRFSGSGSGTSYSLTISRVEAEDAATYYCQQWTSNPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 564 - protein, 448 aa, synthetic construct
QAYLQQSGAELVRPGASVKMSCKASGYTFTSYNMHWVKQTPRQGLEWIGGIYPGNGDTSYNQKFKGKATLTVGKSSSTAYMQLSSLTSEDSAVYFCARYDYNYAMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 565 - protein, 213 aa, synthetic construct
QIVLSQSPAILSASPGEKVTMTCRASSSVSYMHWYQQKPGSSPKPWIYATSNLASGVPARFSGSGSGTSYSFTISRVEAEDAATYYCQQWTFNPPTFGGGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 566 - protein, 451 aa, synthetic construct
QVQLQQSGAEVKKPGSSVKVSCKASGYTFTSYNMHWVKQAPGQGLEWIGAIYPGMGDTSYNQKFKGKATLTADESTNTAYMELSSLRSEDTAFYYCARSTYYGGDWYFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 567 - protein, 213 aa, synthetic construct
DIQLTQSPSSLSASVGDRVTMTCRASSSVSYIHWFQQKPGKAPKPWIYATSNLASGVPVRFSGSGSGTDYTFTISSLQPEDIATYYCQQWTSNPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 568 - protein, 452 aa, synthetic construct
EVQLVESGGGLVQPGGSLRLSCAASGYTFTSYNMHWVRQAPGKGLEWVGAIYPGNGDTSYNQKFKGRFTISVDKSKNTLYLQMNSLRAEDTAVYYCARVVYYSNSYWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 569 - protein, 213 aa, synthetic construct; identical to SEQ ID NO: 559

SEQ ID NO: 570 - protein, 451 aa, synthetic construct; identical to SEQ ID NO: 151

SEQ ID NO: 571 - protein, 214 aa, synthetic construct; identical to SEQ ID NO: 152

SEQ ID NO: 572 - protein, 674 aa, synthetic construct
QVQLVQSGAEVKKPGSSVKVSCKASGYAFSYSWINWVRQAPGQGLEWMGRIFPGDGDTDYNGKFKGRVTITADKSTSTAYMELSSLRSEDTAVYYCARNVFDGYWLVYWGQGTLVTVSSASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDEKVEPKSCDGGGGSGGGGSQAVVTQEPSLTVSPGGTVTLTCGSSTGAVTTSNYANWVQEKPGQAFRGLIGGTNKRAPGTPARFSGSLLGGKAALTLSGAQPEDEAEYYCALWYSNLWVFGGGTKLTVLSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 573 - protein, 449 aa, synthetic construct; identical to SEQ ID NO: 160

SEQ ID NO: 574 - protein, 219 aa, synthetic construct
DIVMTQTPLSLPVTPGEPASISCRSSKSLLHSNGITYLYWYLQKPGQSPQLLIYQMSNLVSGVPDRFSGSGSGTDFTLKISRVEAEDVGVYYCAQNLELPYTFGGGTKVEIKRTVAAPSVFIFPPSDRKLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 575 - protein, 453 aa, synthetic construct
EVQLVESGGGLVQPGRSLRLSCVASGFTFNDYAMHWVRQAPGKGLEWVSVISWNSDSIGYADSVKGRFTISRDNAKNSLYLQMHSLRAEDTALYYCAKDNHYGSGSYYYYQYGMDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 576 - protein, 446 aa, synthetic construct
QLVQSGAEVKKPGASVKVSCKASGYRFTNYWIHWVRQAPGQGLEWIGGINPGNNYATYRRKFQGRVTMTADTSTSTVYMELSSLRSEDTAVYYCTREGYGNYGAWFAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 577 - protein, 219 aa, synthetic construct
DVQVTQSPSSLSASVGDRVTITCRSSQSLANSYGNTFLSWYLHKPGKAPQLLIYGISNRFSGVPDRFSGSGSGTDFTLTISSLQPEDFATYYCLQGTHQPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 578 - protein, 476 aa, synthetic construct
MEVQLVESGGGLVKPGGSLKLSCAASGFAFSIYDMSWVRQTPEKCLEWVAYISSGGGTTYYPDTVKGRFTISRDNAKNTLYLQMSSLKSEDTAMYYCARHSGYGTHWGVLFAYWGQGTLVTVSAKASGGPEGGSLAALTAHQACHLPLETFTRHRQPRGWEQLEQCGYPVQRLVALYLAARLSWNQVDQVIRNALASPGSGGDLGEAIREQPEQARLALTLAAAESERFVRQGTGNDEAGAANGPADSGDALLERNYPTGAEFLGDGGDVSFSTRGTQNWTVERLLQAHRQLEERGYVFVGYHGTFLEAAQSIVFGGVRARSQDLDAIWRGFYIAGDPALAYGYAQDQEPDARGRIRNGALLRVYVPRSSLPGFYRTSLTLAAPEAAGEVERLIGHPLPLRLDAITGPEEEGGRLETILGWPLAERTVVIPSAIPTDPRNVGGDLDPSSIPDKEQAISALPDYASQPGKPPREDLK

SEQ ID NO: 579 - protein, 108 aa, synthetic construct
MDIQMTQTTSSLSASLGDRVTISCRASQDISNYLNWYQQKPDGTVKLLIYYTSILHSGVPSRFSGSGSGTDYSLTISNLEQEDFATYFCQQGNTLPWTFGCGTKLEIK

SEQ ID NO: 580 - protein, 450 aa, synthetic construct
EVQLVESGGGLVQPGGSLRLSCAASGYEFSRSWMNWVRQAPGKGLEWVGRIYPGDGDTNYSGKFKGRFTISADTSKNTAYLQMNSLRAEDTAVYYCARDGSSWDWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 581 - protein, 219 aa, synthetic construct
DIQMTQSPSSLSASVGDRVTITCRSSQSIVHSVGNTFLEWYQQKPGKAPKLLIYKVSNRFSGVPSRFSGSGSGTDFTLTISSLQPEDFATYYCFQGSQFPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 582 - protein, 446 aa, synthetic construct
QIQLQQSGPEVVKPGASVKISCKASGYTFTDYYITWVKQKPGQGLEWIGWIYPGSGNTKYNEKFKGKATLTVDTSSSTAFMQLSSLTSEDTAVYFCANYGNYWFAYWGQGTQVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 583 - protein, 218 aa, synthetic construct
DIVLTQSPASLAVSLGQRATISCKASQSVDFDGDSYMNWYQQKPGQPPKVLIYAASNLESGIPARFSGSGSGTDFTLNIHPVEEEDAATYYCQQSNEDPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 584 - protein, 446 aa, synthetic construct
QVQLVQSGAEVKKPGSSVKVSCKASGYTFTDYNMHWVRQAPGQGLEWIGYIYPYNGGTGYNQKFKSKATITADESTNTAYMELSSLRSEDTAVYYCARGRPAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 585 - protein, 218 aa, synthetic construct
DIQMTQSPSSLSASVGDRVTITCRASESVDNYGISFMNWFQQKPGKAPKLLIYAASNQGSGVPSRFSGSGSGTDFTLTISSLQPDDFATYYCQQSKEVPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 586 - protein, 447 aa, synthetic construct
QVQLVQSGAEVKKPGASVKVSCKASGYTFTNYDINWVRQAPGQGLEWIGWIYPGDGSTKYNEKFKAKATLTADTSTSTAYMELRSLRSDDTAVYYCASGYEDAMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPCVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 587 - protein, 214 aa, synthetic construct
DIQMTQSPSSLSASVGDRVTINCKASQDINSYLSWFQQKPGKAPKTLIYRANRLVDGVPSRFSGSGSGQDYTLTISSLQPEDFATYYCLQYDEFPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 588 - protein, 443 aa, synthetic construct
EVQLVQSGAEVKKPGSSVKVSCKASGYTITDSNIHWVRQAPGQSLEWIGYIYPYNGGTDYNQKFKNRATLTVDNPTNTAYMELSSLRSEDTAFYYCVNGNPWLAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 589 - protein, 218 aa, synthetic construct
DIQLTQSPSTLSASVGDRVTITCRASESLDNYGIRFLTWFQQKPGKAPKLLMYAASNQGSGVPSRFSGSGSGTEFTLTISSLQPDDFATYYCQQTKEVPWSFGQGTKVEVKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 590 - protein, 443 aa, synthetic construct
EIQLQQSGPELVKPGASVKVSCKASGYSFTDYNMYWVKQSHGKSLEWIGYIDPYNGDTTYNQKFKGKATLTVDKSSSTAFIHLNSLTSEDSAVYYCARSPYGHYAMDYWGQGTSVTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 591 - protein, 214 aa, synthetic construct
DIVMTQSHKLLSTSVGDRVSITCKASQDVSTAVDWYQQKPGQSPKLLINWASTRHTGVPDRFTGSGSGTDYTLTISSMQAEDLALYYCRQHYSTPFTFGSGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 592 - protein, 444 aa, synthetic construct
QVQVQESGPGLVAPSQTLSITCTVSGFSLTTSGVSWVRQPPGKGLEWLGVIWGDGSTNYHPSLKSRLSIKKDHSKSQVFLKLNSLTAADTATYYCAKGGYSLAHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 593 - protein, 214 aa, synthetic construct
DIQMTQSPSSLSVSVGERVTITCRASENIRSNLAWYQQKPGKSPKLLVNVATNLADGVPSRFSGSGSGTDYSLKINSLQPEDFGTYYCQHYWGTTWTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 594 - protein, 483 aa, synthetic construct
EVQLVQSGAEVKKPGESLKISCKGSGYSFTGYNMNWVRQMPGKGLEWMGNIDPYYGGTTYNRKFKGQVTISADKSISTAYLQWSSLKASDTAMYYCARSVGPFDSWGQGTLVTVSSGGGGSGGGGSGGGGSGGGGSGGGGSEIVLTQSPATLSLSPGERATLSCRASENVYSYLAWYQQKPGQAPRLLIYFAKTLAEGIPARFSGSGSGTDFTLTISSLEPEDFAVYYCQHHSDNPWTFGQGTKVEIKGDQEPKSSDKTHTSPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 595 - protein, 449 aa, synthetic construct
EVQLVESGGGLVQPGGSLRLSCAASGFSLSNYNMGWVRQAPGKGLEWVSVIDASGTTYYATWAKGRFTISRDNSKNTLYLQMNSLRAEDTATYYCARELLYFGSSYYDLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFLLYSKLTVDKSRWQQGNVFSCSVMHGALHNHYTQKSLSLSPG

SEQ ID NO: 596 - protein, 217 aa, synthetic construct
DVVMTQSPSTLSASVGDRVTITCQASQNIDSNLAWYQQKPGKAPKFLIYYASNLPFGVPSRFKGSGSGTEFTLTISSLQPDDFATYYCQSADVGSTYVAAFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 597 - protein, 448 aa, synthetic construct
EVQLVESGGGLVQPGGSLRLSCAASGFSLSYNAMNWVRQAPGKGLEWVSIIFASGRTDYASWAKGRFTISRDNSKNTLYLQMNSLRAEDTAVYYCAREGSTWGDALDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSRLTVDKSRWQQGNVFSCSVMHGALHNHYTQKSLSLSPG

SEQ ID NO: 598 - protein, 217 aa, synthetic construct
AYDMTQSPSTLSASVGDRVTITCQASQNIIDYLAWYQQKPGKAPKLLIHKASTLASGVPSRFKGSGSGTEFTLTISSLQPDDFATYYCQQGYSNSNIDNTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 599 - protein, 450 aa, synthetic construct
QVQLVQSGAEVAKPGTSVKLSCKASGYTFTDYWMQWVKQRPGQGLEWIGTIYPGDGDTGYAQKFQGKATLTADKSSKTVYMHLSSLASEDSAVYYCARGDYYGSNSLDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 600 - protein, 214 aa, synthetic construct
DIVMTQSHLSMSTSLGDPVSITCKASQDVSTVVAWYQQKPGQSPRRLIYSASYRYIGVPDRFTGSGAGTDFTFTISSVQAEDLAVYYCQQHYSPPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 601 - protein, 447 aa, synthetic construct
QVQLQQSGGELAKPGASVKVSCKASGYTFSSFWMHWVRQAPGQGLEWIGYINPRSGYTEYNEIFRDKATMTTDTSTSTAYMELSSLRSEDTAVYYCASFLGRGAMDYWGQGTTVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVEPKSSDKTHTCPPCPAPPVAGPSVFLFPPKPKDTLMISRTPEVTCVVVDVSHEDPEVQFNWYVDGVEVHNAKTKPREEQAQSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 602 - protein, 214 aa, synthetic construct
DIQMTQSPSSLSASVGDRVTITCRASQDISNYLAWYQQKPGKAPKLLIYYTSKIHSGVPSRFSGSGSGTDYTFTISSLQPEDIATYYCQQGNTFPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 603 - protein, 449 aa, synthetic construct
QVQLVESGGGVVQPGRSRRLSCAASGFTFSRYTMHWVRQAPGKGLEWVAVISFDGSNKYYVDSVKGRFTISRDNSENTLYLQVNILRAEDTAVYYCAREARGSYAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 604 - protein, 215 aa, synthetic construct
EIVLTQSPATLSLSPGERATLSCRASQSVSSYLAWYQQKPGQAPRLLIYDASNRATGIPARFSGSGSGTDFTLTISSLEPEDFAVYYCQQRSNWPPFTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 605 - protein, 451 aa, synthetic construct
QVQLQESGPGLVRPSQTLSLTCTVSGFTFTDFYMNWVRQPPGRGLEWIGFIRDKAKGYTTEYNPSVKGRVTMLVDTSKNQFSLRLSSVTAADTAVYYCAREGHTAAPFDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 606 - protein, 214 aa, synthetic construct
DIQMTQSPSSLSASVGDRVTITCKASQNIDKYLNWYQQKPGKAPKLLIYNTNNLQTGVPSRFSGSGSGTDFTFTISSLQPEDIATYYCLQHISRPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 607 - protein, 448 aa, synthetic construct; identical to SEQ ID NO: 263

SEQ ID NO: 608 - protein, 219 aa, synthetic construct; identical to SEQ ID NO: 264

SEQ ID NO: 609 - protein, 452 aa, synthetic construct; identical to SEQ ID NO: 276

SEQ ID NO: 610 - protein, 216 aa, synthetic construct; identical to SEQ ID NO: 277

SEQ ID NO: 611 - protein, 448 aa, synthetic construct; identical to SEQ ID NO: 278

SEQ ID NO: 612 - protein, 218 aa, synthetic construct; identical to SEQ ID NO: 279

SEQ ID NO: 613 - protein, 447 aa, synthetic construct; identical to SEQ ID NO: 286

SEQ ID NO: 614 - protein, 216 aa, synthetic construct; identical to SEQ ID NO: 287

SEQ ID NO: 615 - protein, 450 aa, synthetic construct
QVQLQQSGSELKKPGASVKVSCKASGYTFTNYGVNWIKQAPGQGLQWMGWINPNTGEPTFDDDFKGRFAFSLDTSVSTAYLQISSLKADDTAVYFCSRSRGKNEAWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 616 - protein, 219 aa, synthetic construct
DIQLTQSPLSLPVTLGQPASISCRSSQSLVHRNGNTYLHWFQQRPGQSPRLLIYTVSNRFSGVPDRFSGSGSGTDFTLKISRVEAEDVGVYFCSQSSHVPPTFGAGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 617 - protein, 447 aa, synthetic construct
EVQLVESGGGLVQPGGSLRLSCAASGYTFSSYWIEWVRQAPGKGLEWIGEILPGGGDTNYNEIFKGRATFSADTSKNTAYLQMNSLRAEDTAVYYCTRRVPIRLDYWGQGTLVTVSSCSTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 618 - protein, 218 aa, synthetic construct
DIQLTQSPSSLSASVGDRVTITCKASQSVDYEGDSFLNWYQQKPGKAPKLLIYAASNLESGVPSRFSGSGSGTDFTLTISSLQPEDFATYYCQQSNEDPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 619 - protein, 447 aa, synthetic construct
EVQLVESGGGLVQPGGSLRLSCAASGYTFSSYWIEWVRQAPGKGLEWIGEILPGGGDTNYNEIFKGRATFSADTSKNTAYLQMNSLRAEDTAVYYCTRRVPIRLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 620 - protein, 218 aa, synthetic construct; identical to SEQ ID NO: 618

SEQ ID NO: 621 - protein, 457 aa, synthetic construct
QVQLQESGPGLVKPSETLSLTCAVSGGSISGGYGWGWIRQPPGKGLEWIGSFYSSSGNTYYNPSLKSQVTISTDTSKNQFSLKLNSMTAADTAVYYCVRDRLFSVVGMVYNNWFDVWGPGVLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKAEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 622 - protein, 216 aa, synthetic construct
ESALTQPPSVSGAPGQKVTISCTGSTSNIGGYDLHWYQQLPGTAPKLLIYDINKRPSGISDRFSGSKSGTAASLAITGLQTEDEADYYCQSYDSSLNAQVFGGGTRLTVLGQPKAAPTVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 623 - protein, 450 aa, synthetic construct
EVQLVQSGAEVKKPGESLKISCKGSGYSFTDYYMKWARQMPGKGLEWMGDIIPSNGATFYNQKFKGQVTISADKSISTTYLQWSSLKASDTAMYYCARSHLLRASWFAYWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPDVFLFPPKPKDTLMISRTPEVTCVVVDVSHEDPEVQFNWYVDGVEVHNAKTKPREEQFNSTFRVVSVLTVVHQDWLNGKEYKCKVSNKALPAPEEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 624 - protein, 220 aa, synthetic construct
DIVMTQSPDSLAVSLGERATINCESSQSLLNSGNQKNYLTWYQQKPGQPPKPLIYWASTRESGVPDRFSGSGSGTDFTLTISSLQAEDVAVYYCQNDYSYPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 625 - protein, 452 aa, synthetic construct
QVQLVQSGAEVKKPGASVKVSCKASGYTFTSYWMNWVRQAPGQGLEWMGRIDPYDSETHYAQKLQGRVTMTTDTSTSTAYMELRSLRSDDTAVYYCARGGYDFDVGTLYWFFDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 626 - protein, 214 aa, synthetic construct
DIQMTQSPSSLSASVGDRVTITCRASENIYSYLAWYQQKPGKAPKLLIYNAKTLAEGVPSRFSGSGSGTDFTLTISSLQPEDFATYYCQHHYGTPRTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 627 - protein, 454 aa, synthetic construct
QVQLQESGPGLVRPSQTLSLTCTASGYTFTDYVIHWVKQPPGRGLEWIGYINPYDDDTTYNQKFKGRVTMLVDTSSNTAYLRLSSVTAEDTAVYYCARRGNSYDGYFDYSMDYWGSGTPVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGFFFLYSKLTVDKSRWQQGNVFSCSVMHEALHNHYTQKSLSLSPGK

SEQ ID NO: 628 - protein, 215 aa, synthetic construct
DIQMTQSPSSLSASVGDRVTITCRASQNVGTAVAWLQQTPGKAPKLLIYSASNRYTGVPSRFSGSGSGTDYTFTISSLQPEDIATYYCQQYTNYPMYTFGQGTKVQIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 629 - protein, 452 aa, synthetic construct; identical to SEQ ID NO: 95

SEQ ID NO: 630 - protein, 214 aa, synthetic construct; identical to SEQ ID NO: 96

SEQ ID NO: 631 - protein, 442 aa, synthetic construct; identical to SEQ ID NO: 97

SEQ ID NO: 632 - protein, 214 aa, synthetic construct; identical to SEQ ID NO: 98

SEQ ID NO: 633 - protein, 449 aa, synthetic construct
EVQLVESGGGLVQPGGSLRLSCAASGFDFSRYWMSWVRQAPGKGLEWIGEINPDSSTINYAPSLKDKFIISRDNAKNSLYLQMNSLRAEDTAVYYCARPDGNYWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 634 - protein, 214 aa, synthetic construct
DIQMTQSPSSLSASVGDRVTITCKASQDVGIAVAWYQQKPGKVPKLLIYWASTRHTGVPDRFSGSGSGTDFTLTISSLQPEDVATYYCQQYSSYPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 635 - protein, 447 aa, synthetic construct
EVQLVESGGGLVQPGGSLRLSCAASGFTFSDYYMAWVRQAPGKGLEWVASINYDGSSTYYVDSVKGRFTISRDNAKNSLYLQMNSLRAEDTAVYYCARDRGYYFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 636 - protein, 220 aa, synthetic construct
DVVMTQTPLSLSVTPGQPASISCRSSQSLVHSNGNTYLHWYLQKPGQSPQLLIYKVSNRFSGVPDRFSGSGSGTDFTLKISRVEAEDVGVYFCSQSTHVPPFTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 637 - protein, 448 aa, synthetic construct
QVQLQQPGAELVRPGASVKLSCKASGYTFTSYWINWVKQRPGQGLEWIGNIYPSDSYTNYNQKFKDKATLTVDKSSSTAYMQLSSPTSEDSAVYYCTRSWRGNSFDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 638-639 - skipped (2 entries)

SEQ ID NO: 640 - protein, 220 aa, synthetic construct
DIVMTQSPSSLTVTAGEKVTMSCKSSQSLLNSGNQKNYLTWYQQKPGQPPKLLIYWASTRESGVPDRFTGSGSGTDFTLTISSVQAEDLAVYYCQNDYSYPFTFGSGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 641 - protein, 447 aa, synthetic construct
QVQLVQSGAEVKKPGASVKVSCKASGYTFTSYYMHWVRQAPGQGLEWMGIINPSGGSTSYAQKFQGRVTMTRDTSTSTVYMELSSLRSEDTAVYYCAKGTTGDWFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELGRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTDPPSREEMTKNQVSLTCEVKGFYPSDIAVEWESNGQPENNYKTTPPVLDSDGSFFLYSKLTVDKSRWQQGNVFSCSVMHEALHNHYTQKSLSLSPG

SEQ ID NO: 642 - protein, 451 aa, synthetic construct
EVQLVESGGGLVQPGGSLRLSCAAAGFTFSSYSMNWVRQAPGKGLEWVSYISSRSRTIYYADSVKGRFTISRDNAKNSLYLQMNSLRDEDTAVYYCARDYGGQPPYYYYYGMDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 643 - protein, 214 aa, synthetic construct
DIQMTQSPSSLSASVGDRVTITCRASQGISSWLAWYQQKPEKAPKSLIYAASSLQSGVPSRFSGSGSGTDFTLTISSLQPEDFVTYYCQQYNSYPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 644 - protein, 447 aa, synthetic construct
QVQLVQSGAEVKKPGASVKVSCKASGYTFTNYGMNWVRQAPGQGLEWMGWINTYTGEPTYADDFKGRVTMTTDTSTSTAYMELRSLRSDDTAVYYCARIGDSSPSDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 645 - protein, 214 aa, synthetic construct
EIVMTQSPATLSVSPGERATLSCKASQSVSNDVVWYQQKPGQAPRLLIYYASNRYTGIPARFSGSGSGTEFTLTISSLQSEDFAVYYCQQDYTSPWTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 646 - protein, 444 aa, synthetic construct
QVQLVQSGAEVKKPGASVKISCKASGYSFTAYYIHWVKQAPGQGLEWIGYISSYNGATNYNQKFKGRVTFTTDTSTSTAYMELRSLRSDDTAVYYCARDYDYDVGMDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPG

SEQ ID NO: 647 - protein, 218 aa, synthetic construct
DIVMTQSPDSLAVSLGERATISCRASESVDNYGISFMKWFQQKPGQPPKLLIYAASNQGSGVPDRFSGSGSGTDFTLTISSLQAEDVAVYYCQQSKEVPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 648 - protein, 452 aa, synthetic construct
QVQLVESGGGVVQPGRSLRLSCAASGFTFSSYGMHWVRQAPGKGLEWVSFLWYDGTNKNYVESVKGRFTISRDNSKNMLYLEMNSLRAEDTAVYYCARDHDFRSGYEGWFDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 649 - protein, 214 aa, synthetic construct
EIVLTQSPATLSLSPGERATLSCRASQSVSSYLAWYQQKPGQAPRLLIYDASNRATGIPARFSGSGSGTDFTLTISSLEPEDFAVYYCQHRSNWPPTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 650 - protein, 445 aa, synthetic construct
QVQLVQSGAEVKKPGASVKISCKASGYSFTAYYIHWVKQAPGQGLEWIGYISNYNRATNYNQKFKGRVTFTTDTSTSTAYMELRSLRSDDTAVYYCARDYDYDVGMDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVEGFYPSDIAVEWESNGQPENNYKTTPPMLDSDGSFFLYSELTVDKSRWQQGNVFSCSVMHEALHNHYTQKSLSLSPGK

SEQ ID NO: 651 - protein, 453 aa, synthetic construct
EVQLVESGGGLVQPGGSLRLSCAASGYTFIDYYMNWVRQAPGKGLEWVGDINLDNSGTHYNQKFKGRFTISRDKSKNTAYLQMNSLRAEDTAVYYCAREGVYHDYDDYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 652 - protein, 219 aa, synthetic construct
DIQMTQSPSSLSASVGDRVTITCRTSQSLVHINAITYLHWYQQKPGKAPKLLIYRVSNRFSGVPSRFSGSGSGTDFTLTISSLQPEDFATYYCGQSTHVPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 653 - protein, 448 aa, synthetic construct
EVKLEESGGGLVQPGGSMKLSCAASGFTFSDAWMDWVRQSPEKGLEWVAEIRSKASNHATYYAESVKGRFTISRDDSKSSVYLQMNSLRAEDTGIYYCTRWRRFFDSWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 654 - protein, 213 aa, synthetic construct
QIVLSQSPAILSASPGEKVTMTCRASSSVSYMHWYQQKPGSSPKPWIYATSNLASGVPVRFSGSGSGTSYSLTISRVEAEDAATYYCQQWSSNPLTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 655 - protein, 457 aa, synthetic construct
EVQLLESGGGVVQPGRSLRLSCAASGFTFSSYGMHWVRQAPGKGLEWVAVISYDGSNKYYADSVKGRFTISRDNSKNTLYLQMNSLRAEDTAVYYCAKDMGWGSGWRPYYYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 656 - protein, 214 aa, synthetic construct
ELQMTQSPSSLSASVGDRVTITCRTSQSISSYLNWYQQKPGQPPKLLIYWASTRESGVPDRFSGSGSGTDFTLTISSLQPEDSATYYCQQSYDIPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 657 - protein, 225 aa, synthetic construct
HHHHHHEVQLVQSGPGLVQPGGSVRISCAASGYTFTNYGMNWVKQAPGKGLEWMGWINTYTGESTYADSFKGRFTFSLDTSASAAYLQINSLRAEDTAVYYCARFAIKGDYWGQGTLLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 658 - protein, 481 aa, synthetic construct
DIQMTQSPSSLSASVGDRVTITCRSTKSLLHSNGITYLYWYQQKPGKAPKLLIYQMSNLASGVPSRFSSSGSGTDFTLTISSLQPEDFATYYCAQNLEIPRTFGQGTKVELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECTRHRQPRGWEQLYNTVSFNLGEAYEYPTFIQDLRNELAKGTPVCQLPVTLQTIADDKRFVLVDITTTSKKTVKVAIDVTDVYVVGYQDKWDGKDRAVFLDKVPTVATSKLFPGVTNRVTLTFDGSYQKLVNAAKADRKALELGVNKLEFSIEAIHGKTINGQEAAKFFLIVIQMVSEAARFKYIETEVVDRGLYGSFKPNFKVLNLENNWGDISDAIHKSSPQCTTINPALQLISPSNDPWVVNKVSQISPDMGILKFKSSK

SEQ ID NO: 659 - protein, 647 aa, synthetic construct
HHHHHHDIQMTQSPSSLSASVGDRVTITCRSTKSLLHSNGITYLYWYQQKPGKAPKLLIYQMSNLASGVPSRFSSSGSGTDFTLTISSLQPEDFATYYCAQNLEIPRTFGQGTKVELKRATPSHNSHQVPSAGGPTANSGTSGSEVQLVQSGPGLVQPGGSVRISCAASGYTFTNYGMNWVKQAPGKGLEWMGWINTYTGESTYADSFKGRFTFSLDTSASAAYLQINSLRAEDTAVYYCARFAIKGDYWGQGTLLTVSSEFGGAPEFPKPSTPPGSSGLEGGSLAALTAHQACHLPLETFTRHRQPRGWEQLEQCGYPVQRLVALYLAARLSWNQVDQVIRNALASPGSGGDLGEAIREQPEQARLALTLAAAESERFVRQGTGNDEAGAASADVVSLTCPVAAGECAGPADSGDALLERNYPTGAEFLGDGGDVSFSTRGTQNWTVERLLQAHRQLEERGYVFVGYHGTFLEAAQSIVFGGVRARSQDLDAIWRGFYIAGDPALAYGYAQDQEPDARGRIRNGALLRVYVPRSSLPGFYRTGLTLAAPEAAGEVERLIGHPLPLRLDAITGPEEEGGRLETILGWPLAERTVVIPSAIPTDPRNVGGDLDPSSIPDKEQAISALPDYASQPGKPPHHHHHHKDEL

SEQ ID NO: 660 - protein, 502 aa, synthetic construct; identical to SEQ ID NO: 189

SEQ ID NO: 661 - protein, 579 aa, synthetic construct; identical to SEQ ID NO: 344

SEQ ID NO: 662 - protein, 213 aa, synthetic construct; identical to SEQ ID NO: 345

SEQ ID NO: 663 - protein, 448 aa, synthetic construct
QVQLVQSGAEVKKPGASVKVSCKASGYTFTGYWMNWVRQAPGQGLEWMGDIYPGSGNTNYDEKFQGRVTMTRDTSISTAYMELSRLRSDDTAVYYCARGGYYEDFDSWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 664 - protein, 214 aa, synthetic construct
DIQMTQSPSFLSASVGDRVTITCRASQGIISYLAWYQQKPEKAPKRLIYAASSLQSGVPSRFSGSGSGTEFTLTISSLQPEDFATYYCGQYANYPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 665 - protein, 455 aa, synthetic construct
QVQLVESGGGVVQPGRSLRLSCAASGFTFSTYGMHWVRQAPGKGLEWVAVIWDDGSYKYYGDSVKGRFTISRDNSKNTLYLQMNSLRAEDTAVYYCARDGITMVRGVMKDYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFLLYSKLTVDKSRWQQGNVFSCSVMHEALHNHYTQKSLSLSPGK

SEQ ID NO: 666 - protein, 214 aa, synthetic construct
AIQLTQSPSSLSASVGDRVTITCRASQDISSALVWYQQKPGKAPKLLIYDASSLESGVPSRFSGSESGTDFTLTISSLQPEDFATYYCQQFNSYPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 667 - protein, 449 aa, synthetic construct
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 668 - protein, 214 aa, synthetic construct
DILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 669 - protein, 446 aa, synthetic construct
QVQLQESGPGLVKPSQTLSLTCTVSGYSISSDFAWNWIRQPPGKGLEWMGYISYSGNTRYQPSLKSRITISRDTSKNQFFLKLNSVTAADTATYYCVTAGRGFPYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 670 - protein, 214 aa, synthetic construct
DIQMTQSPSSMSVSVGDRVTITCHSSQDINSNIGWLQQKPGKSFKGLIYHGTNLDDGVPSRFSGSGSGTDYTLTISSLQPEDFATYYCVQYAQFPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 671 - protein, 452 aa, synthetic construct
EVQLQQPGSELVRPGASVKLSCKASGYTFTSYWMHWVKQRPGQGLEWIGNIYPGSRSTNYDEKFKSKATLTVDTSSSTAYMQLSSLTSEDSAVYYCTRNGDYYVSSGDAMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 672 - protein, 214 aa, synthetic construct
DIQMTQTTSSLSASLGDRVTISCRTSQDIGNYLNWYQQKPDGTVKLLIYYTSRLHSGVPSRFSGSGSGTDFSLTINNVEQEDVATYFCQHYNTVPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 673 - protein, 449 aa, synthetic construct
QVQLVQSGAEVKKPGSSVKVSCKASGFTFTDYKIHWVRQAPGQGLEWMGYFNPNSGYSTYAQKFQGRVTITADKSTSTAYMELSSLRSEDTAVYYCARLSPGGYYVMDAWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 674 - protein, 213 aa, synthetic construct
DIQMTQSPSSLSASVGDRVTITCRASQGINNYLNWYQQKPGKAPKRLIYNTNNLQTGVPSRFSGSGSGTEFTLTISSLQPEDFATYYCLQHNSFPTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 675 - protein, 448 aa, synthetic construct
QVQLVQSGAEVAKPGASVKLSCKASGYTFTSYWMQWVKQRPGQGLECIGTIYPGDGDTTYTQKFQGKATLTADKSSSTAYMQLSSLRSEDSAVYYCARYDAPGYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 676 - protein, 214 aa, synthetic construct
DIQMTQSPSSLSASVGDRVTITCRASQDINNYLAWYQHKPGKGPKLLIHYTSTLHPGIPSRFSGSGSGRDYSFSISSLEPEDIATYYCLQYDNLLYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 677 - protein, 446 aa, synthetic construct
EVQLQESGPGLVKPSQTLSLTCTVSGYSISRDFAWNWIRQPPGKGLEWMGYISYNGNTRYQPSLKSRITISRDTSKNQFFLKLNSVTAADTATYYCVTASRGFPYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 678 - protein, 214 aa, synthetic construct; identical to SEQ ID NO: 670

SEQ ID NO: 679 - protein, 448 aa, synthetic construct
QVQLQQPGAELVEPGGSVKLSCKASGYTFTSHWMHWVKQRPGQGLEWIGEINPSSGRNNYNEKFKSKATLTVDKSSSTAYMQFSSLTSEDSAVYYCVRYYGYDEAMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 680 - protein, 219 aa, synthetic construct
DIVMTQAAFSNPVTLGTSASISCRSSKSLLHSNGITYLYWYLQKPGQSPQLLIYQMSNLASGVPDRFSSSGSGTDFTLRISRVEAEDVGVYYCAQNLELPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 681 - protein, 451 aa, synthetic construct
QVQLQESGPGLVKPSQTLSLTCTVSGGSISSGDYYWSWIRQPPGKGLEWIGYIYYSGSTDYNPSLKSRVTMSVDTSKNQFSLKVNSVTAADTAVYYCARVSIFGVGTFDYWGQGTLVTVSSASTKGPSVLPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 682 - protein, 214 aa, synthetic construct
EIVMTQSPATLSLSPGERATLSCRASQSVSSYLAWYQQKPGQAPRLLIYDASNRATGIPARFSGSGSGTDFTLTISSLEPEDFAVYYCHQYGSTPLTFGGGTKAEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 683 - protein, 453 aa, synthetic construct
QVQLQQSGAEVKKPGSSVKVSCKASGYTFTNYYIYWVRQAPGQGLEWIGGINPTSGGSNFNEKFKTRVTITVDESTNTAYMELSSLRSEDTAFYFCARQGLWFDSDGRGFDFWGQGSTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 684 - protein, 219 aa, synthetic construct
DIQMTQSPSSLSASVGDRVTITCRSSQNIVHSNGNTYLDWYQQTPGKAPKLLIYKVSNRFSGVPSRFSGSGSGTDFTFTISSLQPEDIATYYCFQYSHVPWTFGQGTKLQITRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 685 - protein, 445 aa, synthetic construct
QVQLQESGPGLVKPSETLSLTCTVSGGSVSSGDYYWTWIRQSPGKGLEWIGHIYYSGNTNYNPSLKSRLTISIDTSKTQFSLKLSSVTAADTAIYYCVRDRVTGAFDIWGQGTMVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 686 - protein, 214 aa, synthetic construct
DIQMTQSPSSLSASVGDRVTITCQASQDISNYLNWYQQKPGKAPKLLIYDASNLETGVPSRFSGSGSGTDFTFTISSLQPEDIATYFCQHFDHLPLAFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 687 - protein, 449 aa, synthetic construct
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSTASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEGLHNHYTQKSLSLSPGK

SEQ ID NO: 688 - protein, 214 aa, synthetic construct; identical to SEQ ID NO: 668

SEQ ID NO: 689 - protein, 455 aa, synthetic construct
QVQLVESGGGVVQPGRSLRLSCAASGFTFSTYGMHWVRQAPGKGLEWVAVIWDDGSYKYYGDSVKGRFTISRDNSKNTLYLQMNSLRAEDTAVYYCARDGITMVRGVMKDYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 690 - protein, 214 aa, synthetic construct; identical to SEQ ID NO: 666

SEQ ID NO: 691 - protein, 444 aa, synthetic construct
QVQLVQSGAEVKKPGSSVKVSCKASGYIFTTYNVHWVRQAPGQGLEWIGSIYPDNGDTSYNQNFKGRATITADKSTSTAYMELSSLRSEDTAVYYCARGDFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 692 - protein, 214 aa, synthetic construct
DIQMTQSPSSLSASVGDRVTITCKASQGVSNDVAWYQQKPGKAPKLLIYSASYRYTGVPSRFSGSGSGTDFTFTISSLQPEDIATYYCQQHSTTPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 693 - protein, 357 aa, synthetic construct
EVQLLESGGGLVQPGGSLRLSCAASGFTFSSFSMSWVRQAPGKGLEWVSSISGSSGTTYYADSVKGRFTISRDNSKNTLYLQMNSLRAEDTAVYYCAKPFPYFDYWGQGTLVTVSSGDGSSGGSGGASEIVLTQSPGTLSLSPGERATLSCRASQSVSSSFLAWYQQKPGQAPRLLIYYASSRATGIPDRFSGSGSGTDFTLTISRLEPEDFAVYYCQQTGRIPPTFGQGTKVEIKSGGSGGPRAAPEVYAFATPEWPGSRDKRTLACLIQNFMPEDISVQWLHNEVQLPDARHSTTQPRKTKGSGFFVFSRLEVTRAEWEQKDEFICRAVHEAASPSQTVQRAVSVNPESSRRGGC

SEQ ID NO: 694 - protein, 449 aa, synthetic construct
EVQLVESGGGVVQPGRSLRLSCSASGFTFSGYGLSWVRQAPGKGLEWVAMISSGGSYTYYADSVKGRFAISRDNAKNTLFLQMDSLRPEDTGVYFCARHGDDPAWFAYWGQGTPVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 695 - protein, 217 aa, synthetic construct
DIQLTQSPSSLSASVGDRVTITCSVSSSISSNNLHWYQQKPGKAPKPWIYGTSNLASGVPSRFSGSGSGTDYTFTISSLQPEDIATYYCQQWSSYPYMYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 696 - protein, 447 aa, synthetic construct
QVQLVQSGAEVVKPGASVKISCKASGYTFTGYFMNWVKQSPGQSLEWIGRIHPYDGDTFYNQKFQGKATLTVDKSSNTAHMELLSLTSEDFAVYYCTRYDGSRAMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 697 - protein, 218 aa, synthetic construct
DIVLTQSPLSLAVSLGQPAIISCKASQSVSFAGTSLMHWYHQKPGQQPRLLIYRASNLEAGVPDRFSGSGSKTDFTLTISPVEAEDAATYYCQQSREYPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 698 - protein, 443 aa, synthetic construct
EVQLVESGGGLVQPGGSLRLSCAASGFTFSHYTLSWVRQAPGKGLEWVSVISGDGSYTYYADSVKGRFTISSDNSKNTLYLQMNSLRAEDTAVYYCARNFIKYVFANWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPG

SEQ ID NO: 699 - protein, 213 aa, synthetic construct
DIELTQPPSVSVAPGQTARISCSGDNIGSFYVHWYQQKPGQAPVLVIYDKSNRPSGIPERFSGSNSGNTATLTISGTQAEDEADYYCQSYANTLSLVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 700 - protein, 445 aa, synthetic construct
QVQLVQSGAEVKKPGASVKVSCKASGYSFTGYTMNWVRQAPGQGLEWMGLINPYNSDTNYAQKLQGRVTMTTDTSTSTAYMELRSLRSDDTAVYYCARVALRVALDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 701 - protein, 214 aa, synthetic construct
DIQMTQSPSSLSASVGDRVTITCKASQNVATHVGWYQQKPGKAPKRLIYSASYRYSGVPSRFSGSGSGTEFTLTISNLQPEDFATYYCQQYNRYPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 702 - protein, 443 aa, synthetic construct; identical to SEQ ID NO: 326

SEQ ID NO: 703 - protein, 220 aa, synthetic construct; identical to SEQ ID NO: 327

SEQ ID NO: 704 - protein, 449 aa, synthetic construct; identical to SEQ ID NO: 330

SEQ ID NO: 705 - protein, 211 aa, synthetic construct; identical to SEQ ID NO: 331

SEQ ID NO: 706 - protein, 449 aa, synthetic construct
EVTLVESGGDFVKPGGSLKVSCAASGFAFSHYAMSWVRQTPAKRLEWVAYISSGGSGTYYSDSVKGRFTISRDNAKNTLYLQMRSLRSEDSAMYFCTRVKLGTYYFDSWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 707 - protein, 214 aa, synthetic construct
DIQMTQTASSLPASLGDRVTISCSASQDISNYLNWYQQKPDGTVKLLIFYSSNLHSGVPSRFSGGGSGTDYSLTISNLEPEDIATYFCHQYSKLPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 708 - protein, 442 aa, synthetic construct
QVQLQESGPGLVKPSETLSLTCTVSGFSLLSYGVHWVRQPPGKGLEWLGVIWTGGTTNYNSALMSRFTISKDDSKNTVYLKMNSLKTEDTAIYYCARYYYGMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 709 - protein, 214 aa, synthetic construct
DIQMTQSPSSLSASVGDRVTITCKASQDVRNTVAWYQQKPGKAPKLLIYSSSYRNTGVPDRFSGSGSGTDFTLTISSLQAEDVAVYYCQQHYITPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 710 - protein, 445 aa, synthetic construct
QVQLVQSGAEVKKPGASVKVSCKASGYTFTDYEMHWVRQAPGQGLEWMGALDPKTGDTAYSQKFKGRVTLTADKSTSTAYMELSSLTSEDTAVYYCTRFYSYTYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 711 - protein, 219 aa, synthetic construct
DVVMTQSPLSLPVTPGEPASISCRSSQSLVHSNRNTYLHWYLQKPGQSPQLLIYKVSNRFSGVPDRFSGSGSGTDFTLKISRVEAEDVGVYYCSQNTHVPPTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 712 - protein, 449 aa, synthetic construct
QVQLQQWGAGLLKPSETLSLTCAVFGGSFSGYYWSWIRQPPGKGLEWIGEINHRGNTNDNPSLKSRVTISVDTSKNQFALKLSSVTAADTAVYYCARERGYTYGNFDHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 713 - protein, 214 aa, synthetic construct
EIVMTQSPATLSVSPGERATLSCRASQSVSRNLAWYQQKPGQAPRLLIYGASTRATGIPARFSGSGSGTEFTLTIGSLQSEDFAVYYCQQYKTWPRTFGQGTNVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 714 - protein, 441 aa, synthetic construct
QVQLVQSGAEVKKPGASVKVSCKASGYTFTDYYMHWVRQAPGQGLEWMGRVNPNRRGTTYNQKFEGRVTMTTDTSTSTAYMELRSLRSDDTAVYYCARANWLDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 715 - protein, 215 aa, synthetic construct
DIQMTQSPSSLSASVGDRVTITCSVSSSVSSIYLHWYQQKPGKAPKLLIYSTSNLASGVPSRFSGSGSGTDFTLTISSLQPEDFATYYCQVYSGYPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 716 - protein, 449 aa, synthetic construct
QVQLVQSGAEVKKPGASVKVSCETSGYTFTSYGISWVRQAPGHGLEWMGWISAYNGYTNYAQKLQGRVTMTTDTSTSTAYMELRSLRSDDTAVYYCARDLRGTNYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSRLTVDKSRWQQGNVFSCSVMHEALHNHYTQKSLSLSPGK

SEQ ID NO: 717 - protein, 214 aa, synthetic construct
DIQMTQSPSSVSASVGDRVTITCRASQGISNWLAWFQHKPGKAPKLLIYAASSLLSGVPSRFSGSGSGTDFTLTISSLQPEDFATYYCQQANSFPITFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 718 - protein, 448 aa, synthetic construct
QVQLVQPGAEVKKPGTSVKLSCKASGYTFTTYWMHWVRQAPGQGLEWIGEINPTNGHTNYNQKFQGRATLTVDKSTSTAYMELSSLRSEDTAVYYCARNYVGSIFDYWGQGTLLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 719 - protein, 214 aa, synthetic construct
DIVMTQSPDSLAMSLGERVTLNCKASENVVSYVSWYQQKPGQSPKLLIYGASNRESGVPDRFSGSGSATDFTLTISSVQAEDVADYHCGQSYNYPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 720 - protein, 449 aa, synthetic construct
EVQLVESGGGLVQPGGSLRLSCAASGYTFTSYWLHWVRQAPGKGLEWVGMIDPSNSDTRFNPNFKDRFTISADTSKNTAYLQMNSLRAEDTAVYYCATYRSYVTPLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 721 - protein, 220 aa, synthetic construct
DIQMTQSPSSLSASVGDRVTITCKSSQSLLYTSSQKNYLAWYQQKPGKAPKLLIYWASTRESGVPSRFSGSGSGTDFTLTISSLQPEDFATYYCQQYYAYPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 722 - protein, 446 aa, synthetic construct
QVQLQESGPGLVKPSETLSLTCTVSGGSISIYYWSWIRQPPGKGLEWIGYVYYSGSTNYNPSLKSRVTISVDTSKNQFSLKLNSVTAADTAVYYCARGGYDFWSGYFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 723 - protein, 215 aa, synthetic construct
EIVMTQSPATLSVSPGERATLSCRASQSVDSNLAWYRQKPGQAPRLLIYGASTRATGIPARFSGSGSGTEFTLTISSLQSEDFAVYYCQQYINWPPITFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 724 - protein, 445 aa, synthetic construct
QVQLVQSGAEVKKPGASVKVSCKASGYIFTAYTMHWVRQAPGQGLEWMGWIKPNNGLANYAQKFQGRVTMTRDTSISTAYMELSRLRSDDTAVYYCARSEITTEFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDCHCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 725 - protein, 218 aa, synthetic construct
DIVMTQSPDSLAVSLGERATINCKSSESVDSYANSFLHWYQQKPGQPPKLLIYRASTRESGVPDRFSGSGSGTDFTLTISSLQAEDVAVYYCQQSKEDPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 726 - protein, 251 aa, synthetic construct
QVQLVESGGGLVQPGGSLRLSCAASGFTFRSYAMSWVRQAPGKGLEWVSAISGRGDNTYYADSVKGRFTISRDNSKNTLYLQMNSLRAEDTAVYYCAKMTSNAFAFDYWGQGTLVTVSSGGGGSGGGGSGGGGSQSVLTQPPSVSGAPGQRVTISCTGSSSNIGAGYGVHWYQQLPGTAPKLLIYGNTNRPSGVPDRFSGFKSGTSASLAITGLQAEDEADYYCQSYDSSLSGWVFGGGTKLTVLGGSGGC

SEQ ID NO: 727 - protein, 450 aa, synthetic construct
QVQLQQSGPELVKPGASLKLSCTASGFNIKDTYIHWVKQRPEQGLEWIGRIYPTNGYTRYDPKFQDKATITADTSSNTAYLQVSRLTSEDTAVYYCSRWGGDGFYAMDYWGQGASVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELVGGPSVFLLPPKPKDTLMISRTPEVTCVVVDVSHEDPEVKFNWYVDGVEVHNAKTKPREEQYNSTLRVVSVLTVLHQDWLNGKEYKCKVSNKALPAPIEKTISKAKGQPREPQVYTLPPSRDELTKNQVSLTCLVKGFYPSDIAVEWESNGQPENNYKTTPLVLDSDGSFFLYSKLTVDKSRWQQGNVFSCSVMHEALHNHYTQKSLSLSPGK

SEQ ID NO: 728 - protein, 214 aa, synthetic construct
DIVMTQSHKFMSTSVGDRVSITCKASQDVNTAVAWYQQKPGHSPKLLIYSASFRYTGVPDRFTGSRSGTDFTFTISSVQAEDLAVYYCQQHYTTPPTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 729 - protein, 448 aa, synthetic construct
EVQLVESGGGLVQPGGSLRLSCAASGFTFTDYTMDWVRQAPGKGLEWVADVNPNSGGSIYNQRFKGRFTLSVDRSKNTLYLQMNSLRAEDTAVYYCARNLGPSFYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 730 - protein, 214 aa, synthetic construct
DIQMTQSPSSLSASVGDRVTITCKASQDVSIGVAWYQQKPGKAPKLLIYSASYRYTGVPSRFSGSGSGTDFTLTISSLQPEDFATYYCQQYYIYPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 731 - protein, 450 aa, synthetic construct
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEGLHNHYTQKSLSLSPGK

SEQ ID NO: 732 - protein, 214 aa, synthetic construct; identical to SEQ ID NO: 61

SEQ ID NO: 733 - protein, 449 aa, synthetic construct
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 734 - protein, 214 aa, synthetic construct; identical to SEQ ID NO: 61

SEQ ID NO: 735 - protein, 451 aa, synthetic construct
EVQLVESGGGLVQPGGSLRLSCAASGFTLSGDWIHWVRQAPGKGLEWVGEISAAGGYTDYADSVKGRFTISADTSKNTAYLQMNSLRAEDTAVYYCARESRVSFEAAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 736 - protein, 214 aa, synthetic construct
DIQMTQSPSSLSASVGDRVTITCRASQNIATDVAWYQQKPGKAPKLLIYSASFLYSGVPSRFSGSGSGTDFTLTISSLQPEDFATYYCQQSEPEPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 737 - protein, 447 aa, synthetic construct
EVQLLESGGGLVQPGGSLRLSCAASGFTFSSYAMSWVRQAPGKGLEWVSAINSQGKSTYYADSVKGRFTISRDNSKNTLYLQMNSLRAEDTAVYYCARWGDEGFDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 738 - protein, 214 aa, synthetic construct
DIQMTQSPSSLSASVGDRVTITCRASQGISNWLAWYQQKPGKAPKLLIYGASSLQSGVPSRFSGSGSGTDFTLTISSLQPEDFATYYCQQYSSFPTTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 739 - protein, 449 aa, synthetic construct
QVQLVQSGAEVKKPGASVKVSCKASGYTFRSSYISWVRQAPGQGLEWMGWIYAGTGSPSYNQKLQGRVTMTTDTSTSTAYMELRSLRSDDTAVYYCARHRDYYSNSLTYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 740 - protein, 220 aa, synthetic construct
DIVMTQSPDSLAVSLGERATINCKSSQSVLNSGNQKNYLTWYQQKPGQPPKLLIYWASTRESGVPDRFSGSGSGTDFTLTISSLQAEDVAVYYCQSDYSYPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 741 - protein, 447 aa, synthetic construct
QVQLQQWGAGLLKPSETLSLTCAVYGGSFSGYYWSWIRQPPGKGLEWIGEINHSGSTNYNPSLKSRVTISVETSKNQFSLKLSSVTAADTAVYYCARDKWTWYFDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 742 - protein, 220 aa, synthetic construct
DIEMTQSPDSLAVSLGERATINCRSSQSVLYSSSNRNYLAWYQQNPGQPPKLLIYWASTRESGVPDRFSGSGSGTDFTLTISSLQAEDVAVYYCQQYYSTPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 743 - protein, 445 aa, synthetic construct
EVQLLESGGGLVQPGGSLRLSCAASGFTFSHYVMAWVRQAPGKGLEWVSSISSSGGWTLYADSVKGRFTISRDNSKNTLYLQMNSLRAEDTAVYYCTRGLKMATIFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 744 - protein, 217 aa, synthetic construct
QSALTQPASVSGSPGQSITISCTGTSSDVGSYNVVSWYQQHPGKAPKLIIYEVSQRPSGVSNRFSGSKSGNTASLTISGLQTEDEADYYCCSYAGSSIFVIFGGGTKVTVLGQPKAAPSVTLFPPSSEELQANKATLVCLVSDFYPGAVTVAWKADGSPVKVGVETTKPSKQSNNKYAASSYLSLTPEQWKSHRSYSCRVTHEGSTVEKTVAPAECS

SEQ ID NO: 745 - protein, 453 aa, synthetic construct
QVQLVQSGAEVKKPGASVKVSCKASGYTFTGYYMHWVRQAPGQGLEWMGWINPNSGGTNYAQKFQGRVTMTRDTSISTAYMELSRLRSDDTAVYYCARDHGSRHFWSYWGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTKPPSREEMTKNQVSLKCLVKGFYPSDIAVEWESNGQPENNYKTTPPVLDSDGSFFLYSKLTVDKSRWQQGNVFSCSVMHEALHNHYTQKSLSLSPG

SEQ ID NO: 746 - protein, 214 aa, synthetic construct
DIQMTQSPSSLSASVGDRVTITCRASQSISSYLNWYQQKPGKAPKLLIYAASSLQSGVPSRFSGSGSGTDFTLTISSLQPEDFATYYCQQSYSTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 747 - protein, 460 aa, synthetic construct
EVQLVQSGAEVKKPGSSVKVSCKASGGTFSSYAISWVRQAPGQGLEWMGGIIPIFGTANYAQKFQGRVTITADKSTSTAYMELSSLRSEDTAVYYCARAPLRFLEWSTQDHYYYYYMDVWGKGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 748 - protein, 214 aa, synthetic construct
SSELTQDPAVSVALGQTVRITCQGDSLRSYYATWYQQKPGQAPILVIYGENKRPSGIPDRFSGSSSGNTASLTITGAQAEDEADYYCKSRDGSGQHLVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPAECS

SEQ ID NO: 749 - protein, 447 aa, synthetic construct
QVQLQESGPGLVKPSETLSLTCTVSGYSITGGYLWNWIRQPPGKGLEWIGYISYDGTNNYKPSLKDRVTISRDTSKNQFSLKLSSVTAADTAVYYCARYGRVFFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 750 - protein, 219 aa, synthetic construct
DIVMTQSPLSLPVTPGEPASISCRSSQSIVHSNGNTYLQWYLQKPGQSPQLLIYKVSNRLYGVPDRFSGSGSGTDFTLKISRVEAEDVGVYYCFQGSHVPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 751 - protein, 450 aa, synthetic construct
EVQLLESGGGLVQPGGSLRLSCTASGFTFSSYAMNWVRQAPGKGLEWVSAISGSGGTTFYADSVKGRFTISRDNSRTTLYLQMNSLRAEDTAVYYCAKDLGWSDSYYYYYGMDVWGQGTTVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPG

SEQ ID NO: 752 - protein, 214 aa, synthetic construct
DIQMTQFPSSLSASVGDRVTITCRASQGIRNDLGWYQQKPGKAPKRLIYAASRLHRGVPSRFSGSGSGTEFTLTISSLQPEDFATYYCLQHNSYPCSFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 753 - protein, 449 aa, synthetic construct
QVQLQESGPGLVKPSGTLSLTCAVSGGSISSSNWWSWVRQPPGKGLEWIGEIYHSGSTNYNPSLKSRVTISVDKSKNQFSLKLSSVTAADTAVYYCARWTGRTDAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 754 - protein, 219 aa, synthetic construct
DVVMTQSPLSLPVTPGEPASISCRSSQSLLHSNGYNYLDWYLQKPGQSPQLLIYLGSNRASGVPDRFSGSGSGTDFTLKISRVEAEDVGVYYCMQGTHWPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 755 - protein, 448 aa, synthetic construct
EVQLVQSGGGLVKPGGSLRLSCAASGFTFSSFAMHWVRQAPGKGLEWISVIDTRGATYYADSVKGRFTISRDNAKNSLYLQMNSLRAEDTAVYYCARLGNFYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 756 - protein, 214 aa, synthetic construct
EIVLTQSPGTLSVSPGERATLSCRASQSIGSSLHWYQQKPGQAPRLLIKYASQSLSGIPDRFSGSGSGTDFTLTISRLEPEDFAVYYCHQSSRLPHTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 757 - protein, 448 aa, synthetic construct
QVELVESGGGVVQPGRSQRLSCAASGFTFSSYGMHWVRQAPGKGLEWVAIIWFDGSSTYYADSVRGRFTISRDNSKNTLYLQMNSLRAEDTAVYFCARELGRRYFDLWGRGTLVSVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 758 - protein, 215 aa, synthetic construct
EIVLTQSPATLSLSPGERATLSCRASQSVSSYLAWYQQKPGQAPRLLIYDASKRATGIPARFSGSGSGTDFTLTISSLEPEDFAVYYCQQRSKWPPWTFGQGTKVESKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 759 - protein, 447 aa, synthetic construct
QVELVESGGGLVQPGGSLRLSCAASGFTFTSYWMSWVRQAPGKGLELVSSITSYGSFTYYADSVKGRFTISRDNSKNTLYLQMNSLRAEDTAVYYCARNMYTHFDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 760 - protein, 216 aa, synthetic construct
DIVLTQPPSVSGAPGQRVTISCSGSSSNIGSNSVSWYQQLPGTAPKLLIYDNSKRPSGVPDRFSGSKSGTSASLAITGLQSEDEADYYCQSRDTYGYYWVFGGGTKLTVLGQPKAAPSVTLFPPSSEELQANKATLVCLISDFYPGAVTVAWKGDSSPVKAGVETTTPSKQSNNKYAASSYLSLTPEQWKSHRSYSCQVTHEGSTVEKTVAPTECS

SEQ ID NO: 761 - protein, 446 aa, synthetic construct
QVQLVQSGAEVKKPGASVKVSCKASGYTFTSYDINWVRQATGQGLEWMGWMNPNSGNTGYAQKFQGRVTMTRNTSISTAYMELSSLRSEDTAVYYCARDPYYYYYGMDVWGQGTTVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 762 - protein, 217 aa, synthetic construct
QSVLTQPPSVSAAPGQKVTISCSGSSSNIENNHVSWYQQLPGTAPKLLIYDNNKRPSGIPDRFSGSKSGTSATLGITGLQTGDEADYYCETWDTSLSAGRVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 763 - protein, 447 aa, synthetic construct
QVQLVESGGGVVQPGRSLRLSCAASGFTFSSYDMSWVRQAPGKGLEWVAKVSSGGGSTYYLDTVQGRFTISRDNSKNTLYLQMNSLRAEDTAVYYCARHLHGSFASWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 764 - protein, 214 aa, synthetic construct
EIVLTQSPATLSLSPGERATLSCQASQSISNFLHWYQQRPGQAPRLLIRYRSQSISGIPARFSGSGSGTDFTLTISSLEPEDFAVYYCQQSGSWPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 765 - protein, 451 aa, synthetic construct
QVQLKESGPGLVAPSQSLSITCTISGFSLTDYGVHWVRQPPGKGLEWLVVIWSDGSSTYNSALKSRMTIRKDNSKSQVFLIMNSLQTDDSAMYYCARHGTYYGMTTTGDALDYWGQGTSVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 766 - protein, 215 aa, synthetic construct
QIVLTQSPAIMSASLGERVTMTCTASSSVSSNYLHWYQQKPGSAPNLWIYSTSNLASGVPARFSGSGSGTSYSLTISSMEAEDAATYYCHQYLRSPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 767 - protein, 272 aa, synthetic construct; identical to SEQ ID NO: 157

SEQ ID NO: 768 - protein, 280 aa, synthetic construct; identical to SEQ ID NO: 156

SEQ ID NO: 769 - protein, 450 aa, synthetic construct
QVQLVQSGAEVKKPGSSVKVSCKASGGTFSFYAISWVRQAPGQGLEWMGGFIPIFGAANYAQKFQGRVTITADESTSTAYMELSSLRSDDTAVYYCARIPSGSYYYDYDMDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 770 - protein, 214 aa, synthetic construct
EIVLTQSPVTLSLSPGERATLSCRASQSVSSYLAWYQQKPGQAPRLLIYDASNRATGIPARFSGSGSGTDFTLTISSLEPEDFAVYYCQQRSNWMYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 771 - protein, 450 aa, synthetic construct
QVQLVQSGAEVKKPGASVKVSCKASGLTIEDYYMHWVRQAPGQGLEWMGWIDPENGDTEYGPKFQGRVTMTRDTSINTAYMELSRLRSDDTAVYYCAVHNAHYGTWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 772 - protein, 219 aa, synthetic construct
DVVMTQSPLSLPVTLGQPASISCRSSQSLLHSSGNTYLEWYQQRPGQSPRPLIYKISTRFSGVPDRFSGSGSGTDFTLKISRVEAEDVGVYYCFQGSHVPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 773 - protein, 450 aa, synthetic construct
EVQLVQSGAEVKKPGASVKVSCKASGYKFSSYWIEWVKQAPGQGLEWIGEILPGSDTTNYNEKFKDRATFTSDTSINTAYMELSRLRSDDTAVYYCARDRGNYRAWFGYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 774 - protein, 214 aa, synthetic construct
DIQMTQSPSSLSASVGDRVTITCRASQDISNYLNWYQQKPGGAVKFLIYYTSRLHSGVPSRFSGSGSGTDYTLTISSLQPEDFATYFCQQGEALPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 775 - protein, 449 aa, synthetic construct
QVQLQQSGPELEKPGASVKISCKASGYSFTGYTMNWVKQSHGKSLEWIGLITPYNGASSYNQKFRGKATLTVDKSSSTAYMDLLSLTSEDSAVYFCARGGYDGRGFDYWGSGTPVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 776 - protein, 213 aa, synthetic construct
DIELTQSPAIMSASPGEKVTMTCSASSSVSYMHWYQQKSGTSPKRWIYDTSKLASGVPGRFSGSGSGNSYSLTISSVEAEDDATYYCQQWSKHPLTFGSGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 777 - protein, 450 aa, synthetic construct
QVELVQSGAEVKKPGESLKISCKGSGYSFTSYWIGWVRQAPGKGLEWMGIIDPGDSRTRYSPSFQGQVTISADKSISTAYLQWSSLKASDTAMYYCARGQLYGGTYMDGWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 778 - protein, 217 aa, synthetic construct
DIALTQPASVSGSPGQSITISCTGTSSDIGGYNSVSWYQQHPGKAPKLMIYGVNNRPSGVSNRFSGSKSGNTASLTISGLQAEDEADYYCSSYDIESATPVFGGGTKLTVLGQPKAAPSVTLFPPSSEELQANKATLVCLISDFYPGAVTVAWKGDSSPVKAGVETTTPSKQSNNKYAASSYLSLTPEQWKSHRSYSCQVTHEGSTVEKTVAPTECS

SEQ ID NO: 779 - protein, 449 aa, synthetic construct
QVQLQQSGAEVKKPGASVKVSCEASGYTFPSYVLHWVKQAPGQGLEWIGYINPYNDGTQYNEKFKGKATLTRDTSINTAYMELSRLRSDDTAVYYCARGFGGSYGFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 780 - protein, 215 aa, synthetic construct
DIQLTQSPSSLSASVGDRVTMTCSASSSVSSSYLYWYQQKPGKAPKLWIYSTSNLASGVPARFSGSGSGTDFTLTISSLQPEDSASYFCHQWNRYPYTFGGGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 781 - protein, 447 aa, synthetic construct
EVQLVESGGGLVQPGGSMRLSCVASGFPFSNYWMNWVRQAPGKGLEWVGEIRLKSNNYTTHYAESVKGRFTISRDDSKNSLYLQMNSLKTEDTAVYYCTRHYYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEGLHNHYTQKSLSLSPGK

SEQ ID NO: 782 - protein, 219 aa, synthetic construct
DIVMTQSPLSNPVTPGEPASISCRSSKSLLHSNGITYFFWYLQKPGQSPQLLIYQMSNLASGVPDRFSGSGSGTDFTLRISRVEAEDVGVYYCAQNLELPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 783 - protein, 443 aa, synthetic construct
QVQLKESGPDLVAPSQSLSITCTVSGFSLSKFGVNWVRQPPGKGLEWLGVIWGDGSTSYNSGLISRLSISKENSKSQVFLKLNSLQADDTATYYCVKPGGDYWGHGTSVTVSSASTKGPSVFPLAPSSKSTSGGTAALGCLVKDYFPEPVTVSWNSGALTSGVHTFPAVLQSSGPYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTMPPVLDSDGSFFLYSKLTVDKSRWQQGNVFSCSVMHEALHNHYTQKSLSLSPGK

SEQ ID NO: 784 - protein, 213 aa, synthetic construct
QVVLTQSPVIMSASPGEKVTMTCSASSSISYMYWYQQKPGTSPKRWIYDTSKLASGVPARFSGSGSGTSYSLTISNMEAGDAATYYCHQRDSYPWTFGGGTNLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 785 - protein, 447 aa, synthetic construct
EVQLVESGGGLVQPGGSLRLSCAASGFTFSSYNMNWVRQAPGKGLEWVSYISSSSSTIYYADSVKGRFTISRDNAKNSLSLQMNSLRDEDTAVYYCARAYYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 786 - protein, 214 aa, synthetic construct
DIQMTQSPSSVSASVGDRVTITCRASQGISGWLAWYQQKPGKAPKFLIYAASTLQSGVPSRFSGSGSGTDFTLTISSLQPEDFATYYCQQANSFPPTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 787 - protein, 447 aa, synthetic construct
QVQLVQSGAEVKKPGASVKISCKVSGYTLRGYWIEWVRQAPGKGLEWIGQILPGTGRTNYNEKFKGRVTMTADTSTDTAYMELSSLRSEDTAVYYCARFDGNYGYYAMDYWGQGTTVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 788 - protein, 215 aa, synthetic construct
QAVVTQEPSLTVSPGGTVTLTCRSSTGAVTTSNYANWFQQKPGQAPRTLIGGTNNRAPGVPARFSGSLLGGKAALTLSGAQPEDEAEYYCALWYSNHWVFGGGTKLTVLGQPKAAPSVTLFPPSSEELQANKATLVCLVSDFYPGAVTVAWKADGSPVKVGVETTKPSKQSNNKYAASSYLSLTPEQWKSHRSYSCRVTHEGSTVEKTVAPAECS

SEQ ID NO: 789 - protein, 441 aa, synthetic construct
EVQLVESGGGLVQPGGSLRLSCAASGFTFSSSGMSWVRQAPGKGLEWVSVIASSGSNTYYADSVKGRFTISRDNSKNTLYLQMNSLRAEDTAVYYCARSIFYTTWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 790 - protein, 215 aa, synthetic construct
DIVLTQSPATLSLSPGERATLSCRASQSVRSNYLAWYQQKPGQAPRLLIYGASSRATGVPARFSGSGSGTDFTLTISSLEPEDFAVYYCQQYSNFPITFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 791 - protein, 448 aa, synthetic construct
EVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 792 - protein, 214 aa, synthetic construct
DIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 793 - protein, 450 aa, synthetic construct
EVQLLESGGGLVQPGGSLRLSCAASGFTFSSYIMMWVRQAPGKGLEWVSSIYPSGGITFYADTVKGRFTISRDNSKNTLYLQMNSLRAEDTAVYYCARIKLGTVTTVDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 794 - protein, 216 aa, synthetic construct
QSALTQPASVSGSPGQSITISCTGTSSDVGGYNYVSWYQQHPGKAPKLMIYDVSNRPSGVSNRFSGSKSGNTASLTISGLQAEDEADYYCSSYTSSSTRVFGTGTKVTVLGQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 795 - protein, 450 aa, synthetic construct
EVQLVESGGGLVQPGGSLRLSCAASGFSFSDFAMSWVRQAPGKGLEWVATIGRVAFHTYYPDSMKGRFTISRDNSKNTLYLQMNSLRAEDTAVYYCARHRGFDVGHFDFWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 796 - protein, 219 aa, synthetic construct
DIQMTQSPSSLSASVGDRVTITCRSSETLVHSSGNTYLEWYQQKPGKAPKLLIYRVSNRFSGVPSRFSGSGSGTDFTLTISSLQPEDFATYYCFQGSFNPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 797 - protein, 457 aa, synthetic construct
QLQLQESGPGLVKPSETLSLTCTVSGGSINSSSYYWGWLRQSPGKGLEWIGSFFYTGSTYYNPSLRSRLTISVDTSKNQFSLMLSSVTAADTAVYYCARQSTYYYGSGNYYGWFDRWDQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 798 - protein, 214 aa, synthetic construct
EIVLTQSPATLSLSPGERATLSCRASQSVSSYLAWYQQKPGQAPRLLIYDASNRATGIPARFSGSGSGTDFTLTISSLEPEDFAVYYCQQRSNWPPAFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 799 - protein, 446 aa, synthetic construct
QVQLVESGGGLVKPGGSLRLSCAASGFTFSDYYMNWIRQAPGKGLEWVSYISSSGSIIYYADSVKGRFTISRDNAKNSLYLQMNSLRAEDTAVYYCAREGRIAARGMDVWGQGTTVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 800 - protein, 215 aa, synthetic construct
DIQMTQSPSSLSASVGDRVSITCRPSQSFSRYINWYQQKPGKAPKLLIHAASSLVGGVPSRFSGSGSGTDFTLTISSLQPEDFATYYCQQTYSNPPITFGQGTRLEMKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 801 - protein, 448 aa, synthetic construct
QVQLVQSGPEVKKPGASVKVSCKASGYTFTDYAVHWVRQAPGKRLEWIGVISTYNDYTYNNQDFKGRVTMTRDTSASTAYMELSRLRSEDTAVYYCARGNSYFYALDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 802 - protein, 218 aa, synthetic construct
EIVLTQSPATLSLSPGERATLSCRASESVDSYGKSFMHWYQQKPGQAPRLLIYRASNLESGIPARFSGSGSGTDFTLTISSLEPEDFAVYYCQQSNEDPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 803 - protein, 448 aa, synthetic construct
EVQLLESGGGLVQPGGSLRLSCAASGFTFSSYAMSWVRQAPGKGLEWVSGITGSGGSTYYADSVKGRFTISRDNSKNTLYLQMNSLRAEDTAVYYCAKDPGTTVIMSWFDPWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 804 - protein, 215 aa, synthetic construct
EIVLTQSPGTLSLSPGERATLSCRASQSVRGRYLAWYQQKPGQAPRLLIYGASSRATGIPDRFSGSGSGTDFTLTISRLEPEDFAVFYCQQYGSSPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 805 - protein, 447 aa, synthetic construct
QVQLVQSGAEVKKPGASVKVSCKASGYTFTDYSIHWVRQAPGQGLEWIGYIYPSNGDSGYNQKFKNRVTMTRDTSTSTAYMELSRLRSEDTAVYYCATYFANNFDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 806 - protein, 218 aa, synthetic construct
DIQMTQSPSSLSASVGDRVTITCKASQSVDYDGDSYMNWYQQKPGKAPKLLIYAASNLESGVPSRFSGSGSGTDFTLTISPVQAEDFATYYCQQSNEDPLTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 807 - protein, 454 aa, synthetic construct
EVQLVESGGGLVQPGGSLRLSCAVSGYSITSDYAWNWVRQAPGKGLEWVGYISNSGSTSYNPSLKSRFTISRDTSKNTLYLQMNSLRAEDTAVYYCARERNYDYDDYYYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 808 - protein, 220 aa, synthetic construct
DIQMTQSPSSLSASVGDRVTITCKSSQSLLYRSNQKNYLAWYQQKPGKAPKLLIYWASTRESGVPSRFSGSGSGTDFTLTISSLQPEDFATYYCQQYYNYPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 809 - protein, 446 aa, synthetic construct
QVQLVESGGGVVQPGRSLRLSCAASGFTFSSYGMHWVRQAPGKGLEWVAVIWYDGSNQYYADSVKGRFTISRDNSKNTLFLQMHSLRAEDTAVYYCARGLTSGRYGMDVWGQGTTVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 810 - protein, 219 aa, synthetic construct
DIVMTQSPLSLPVTPGEPASISCRSSQSLLLSHGFNYLDWYLQKPGQSPQLLIYLGSSRASGVPDRFSGSGSGTDFTLKISRVEAEDVGLYYCMQPLQIPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 811 - protein, 449 aa, synthetic construct
QVQLQQSGSELMMPGASVKISCKATGYTFSNYWIEWVKQRPGHGLEWIGEILPGTGRTIYNEKFKGKATFTADISSNTVQMQLSSLTSEDSAVYYCARRDYYGNFYYAMDYWGQGTSVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 812 - protein, 214 aa, synthetic construct
DIQMTQSTSSLSASLGDRVTISCSASQGINNYLNWYQQKPDGTVELLIYYTSTLQSGVPSRFSGSGSGTDYSLTISNLEPEDIGTYYCQQYSKLPRTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 813 - protein, 452 aa, synthetic construct
QVQLQESGPGLVKPSQTLSLTCTVSGGSISSGDYFWSWIRQLPGKGLEWIGHIHNSGTTYYNPSLKSRVTISVDTSKKQFSLRLSSVTAADTAVYYCARDRGGDYYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 814 - protein, 215 aa, synthetic construct
EIVLTQSPGTLSLSPGERATLSCRASQGISRSYLAWYQQKPGQAPSLLIYGASSRATGIPDRFSGSGSGTDFTLTISRLEPEDFAVYYCQQFGSSPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 815 - protein, 451 aa, synthetic construct
EVQLVQSGGGVERPGGSLRLSCAASGFTFDDYGMSWVRQAPGKGLEWVSGINWNGGSTGYADSVKGRVTISRDNAKNSLYLQMNSLRAEDTAVYYCAKILGAGRGWYFDLWGKGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 816 - protein, 214 aa, synthetic construct
SSELTQDPAVSVALGQTVRITCQGDSLRSYYASWYQQKPGQAPVLVIYGKNNRPSGIPDRFSGSSSGNTASLTITGAQAEDEADYYCNSRDSSGNHVVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 817 - protein, 449 aa, synthetic construct
EVQLVESGGGLVQPGGSLRLSCAASGFTFSSYVMSWVRQAPGKGLEWVATISSGGSYTYYPDSVKGRFTISRDNAKNTLYLQMNSLRAEDTAVYYCARRGDSMITTDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 818 - protein, 213 aa, synthetic construct
DIQMTQSPSSLSASVGDRVTITCKASQDVGTAVAWYQQKPGKAPKLLIYWASTRHTGVPSRFSGSGSGTDFTLTISSLQPEDFATYYCQQYSSYRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 819 - protein, 445 aa, synthetic construct
QVQLQESGPGLVKPSQTLSLTCTVSGGSISSFNYYWSWIRHHPGKGLEWIGYIYYSGSTYSNPSLKSRVTISVDTSKNQFSLTLSSVTAADTAVYYCARGYNWNYFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 820 - protein, 215 aa, synthetic construct
EIVMTQSPATLSVSPGERATLSCRASQSVDNNLVWYQQKPGQAPRLLIYGASTRATGIPARFSGSGSGTEFTLTISSLQSEDFAVYYCQQYNNWPPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 821 - protein, 458 aa, synthetic construct
EVQLQQSGPDLVKPGASVKISCKASGYSFTGYYMHWVKQSPGKGLEWIGRINPNNGVTLYNQKFKDKATLTVDKSSTTAYMELRSLTSEDSAVYYCARSTMITNYVMDYWGQGTSVTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDSGGPSEKSEEINEKDLRKKSELQGTALGNLKQIYYYNSKAITSSEKSADQFLTNTLLFKGFFTGHPWYNDLLVDLGSTAATSEYEGSSVDLYGAYYGYQCAGGTPNKTACMYGGVTLHDNNRLTEEKKVPINLWIDGKQTTVPIDKVKTSKKEVTVQELDLQARHYLHGKFGLYNSDSFGGKVQRGLIVFHSSEGSTVSYDLFDAQGQYPDTLLRIYRDNTTISSTSLSISLYLYTT

SEQ ID NO: 822 - protein, 214 aa, synthetic construct
SIVMTQTPTSLLVSAGDRVTITCKASQSVSNDVAWYQQKPGQSPKLLISYTSSRYAGVPDRFSGSGYGTDFTLTISSVQAEDAAVYFCQQDYNSPPTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNES

SEQ ID NO: 823 - protein, 451 aa, synthetic construct
QVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYFDVWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 824 - protein, 214 aa, synthetic construct
DIQLTQSPSSLSASVGDRVSITCKASQDVSIAVAWYQQKPGKAPKLLIYSASYRYTGVPDRFSGSGSGTDFTLTISSLQPEDFAVYYCQQHYITPLTFGAGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 825 - protein, 432 aa, synthetic construct
QVQLQQSDAELVKPGASVKISCKASGYTFTDHAIHWAKQKPEQGLEWIGYISPGNDDIKYNEKFKGKATLTADKSSSTAYMQLNSLTSEDSAVYFCKRSYYGHWGQGTTLTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSR

SEQ ID NO: 826 - protein, 210 aa, synthetic construct
DIQMTQSPASLSVSVGETVTITCRASENIYSNLAWYQQKQGKSPQLLVYAATNLADGVPSRFSGSGSGTQYSLKINSLQSEDFGSYYCQHFWGTPYTFGGGTRLEIKRADAAPTVFIFPPSDEQLKSGTASVVCLLNNFYPREAKVQWKVDNALQSGNSQESVTEQDSKDSTYSLSSTLTLSKADYEKHKVYACEVTHQGLSSPVTKSFN

SEQ ID NO: 827 - protein, 449 aa, synthetic construct
EVQLVESGGGLVQPGGSLRLSCAASGFTFSSYWMSWVRQAPGKGLEWVAEIRLKSDNYATHYAESVKGRFTISRDDSKNSLYLQMNSLRAEDTAVYYCTGYYADAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 828 - protein, 218 aa, synthetic construct
DIQMTQSPSSLSASVGDRVTITCRASQSVSTSSYSYMHWYQQKPGKAPKLLIKYASNLESGVPSRFSGSGSGTDFTLTISSLQPEDFATYYCQHSWEIPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 829 - protein, 449 aa, synthetic construct
QVQLVQSGSELKKPGASVKISCKASGYTFTSYAMNWVRQAPGQGLESMGWINTNTGNPTYAQGFTGRFVFSMDTSVSTAYLQISSLKAEDTAIYYCAPRYSSSWYLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 830 - protein, 215 aa, synthetic construct
EIVLTQSPATLSLSPGERATLSCRASQSVSSYLAWYQQKPGQAPRLLIYDASNRATGIPARFSGSGSGTDFTLTISSLEPEDFAVYYCQQRSNWLMYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 831 - protein, 446 aa, synthetic construct
QVQLQESGPGLVKPSQTLSLTCTVSGYAFTAYNIHWVRQAPGQGLEWMGSFDPYDGGSSYNQKFKDRLTISKDTSKNQVVLTMTNMDPVDTATYYCARGWYYFDYWGHGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 832 - protein, 214 aa, synthetic construct
DIVMTQTPLSLPVTPGEPASISCRASKSISKYLAWYQQKPGQAPRLLIYSGSTLQSGIPPRFSGSGYGTDFTLTINNIESEDAAYYFCQQHDESPYTFGEGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 833 - protein, 450 aa, synthetic construct
EVQLLESGGDLVQPGGSLRLSCAASGFTFSNYAMSWVRQAPGKGLEWVSAITPSGGSTNYADSVKGRFTISRDNSQNTLYLQMNSLRVEDTAVYICGRVPYRSTWYPLYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 834 - protein, 214 aa, synthetic construct
DIQMTQSPSSLSASVGDRVTITCRASQDISNYLAWFQQKPGKAPKSLIYAASSLHSKVPTQFSGSGSGTDFTLTISSLQPEDFATYYCLQYSTYPITFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 835 - protein, 449 aa, synthetic construct
EVQLVESGGGVVQPGRSLRLSCSASGFDFTTYWMSWVRQAPGKGLEWIGEIHPDSSTINYAPSLKDRFTISRDNAKNTLFLQMDSLRPEDTGVYFCASLYFGFPWFAYWGQGTPVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 836 - protein, 213 aa, synthetic construct
DIQLTQSPSSLSASVGDRVTITCKASQDVGTSVAWYQQKPGKAPKLLIYWTSTRHTGVPSRFSGSGSGTDFTFTISSLQPEDIATYYCQQYSLYRSFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 837 - protein, 449 aa, synthetic construct
EVQLQESGPGLVKPGGSLSLSCAASGFVFSSYDMSWVRQTPERGLEWVAYISSGGGITYAPSTVKGRFTVSRDNAKNTLYLQMNSLTSEDTAVYYCAAHYFGSSGPFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 838 - protein, 214 aa, synthetic construct
DIQMTQSPASLSASVGDRVTITCRASENIFSYLAWYQQKPGKSPKLLVYNTRTLAEGVPSRFSGSGSGTDFSLTISSLQPEDFATYYCQHHYGTPFTFGSGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 839 - protein, 116 aa, synthetic construct [sequence omitted]

SEQ ID NO: 840 - protein, 112 aa, synthetic construct [sequence omitted]

SEQ ID NO: 841 - protein, 117 aa, synthetic construct
VQLVESGGGLAKPGGSLRLSCAASGFRFTFNNYYMDWVRQAPGQGLEWVSRISSSGDPTWYADSVKGRFTISRENANNTLFLQMNSLRAEDTAVYYCASLTTGSDSWGQGVLVTVSS

SEQ ID NO: 842 - protein, 107 aa, synthetic construct
DIQMTQSPSSLSASVGDRVTITCRASQDIRYYLNWYQQKPGKAPKLLIYVASSLQSGVPSRFSGSGSGTEFTLTVSSLQPEDFATYYCLQVYSTPRTFGQGTKVEIK

SEQ ID NO: 843 - protein, 115 aa, synthetic construct
EVQLVESGGGLVKPGGSLRLSCAASGFTFSSYDMSWVRQAPGKGLEWVSTISSGGSYTYYLDSIKGRFTISRDNAKNSLYLQMNSLRAEDTAVYYCARQGLDYWGRGTLVTVSSA

SEQ ID NO: 844 - protein, 107 aa, synthetic construct
EIVLTQSPATLSLSPGERATLSCSASSSINYIYWYQQKPGQAPRLLIYLTSNLASGVPARFSGSGSGTDFTLTISSLEPEDFAVYYCLQWSSNPLTFGGGTKVEIKR

SEQ ID NO: 845 - protein, 443 aa, synthetic construct
VQLVESGGGLVQPGGSLRLSCAASGFPFSNYWMNWVRQAPGKGLEWVGQIRLKSNNYATHYAESVKGRFTISRDDSKNSLYLQMNSLKTEDTAVYYCTPIDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 846 - protein, 218 aa, synthetic construct
DIVMTQTPLSLSVTPGQPASISCKSSQSLLYSNGKTYLNWVLQKPGQSPQRLIYLVSKLDSGVPDRFSGSGSGTDFTLKISRVEAEDVGVYYCVQGSHFHTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 847 - protein, 451 aa, synthetic construct; identical to SEQ ID NO: 282

SEQ ID NO: 848 - protein, 220 aa, synthetic construct; identical to SEQ ID NO: 283

SEQ ID NO: 849 - protein, 603 aa, synthetic construct; identical to SEQ ID NO: 280

SEQ ID NO: 850 - protein, 214 aa, synthetic construct; identical to SEQ ID NO: 281

SEQ ID NO: 851 - protein, 448 aa, synthetic construct
QVQLVQSGAEVKKPGASVKVSCKASGYTFTGYNMNWVRQAPGQGLEWMGNIDPYYGGTSYNQKFKGRVTMTIDKSTSTVYMELSSLRSEDTAVYYCARMYHGNAFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 852 - protein, 220 aa, synthetic construct
DIVMTQSPDSLAVSLGERATINCKSSQSLLNSGNLKNYLTWYQQKPGQPPKLLIYWASTRKSGVPDRFSGSGSGTDFTLTISSLQAEDVAVYYCQNDYSYPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 853 - protein, 122 aa, synthetic construct
QVQLVQSGAEVKKPGASVKVSCKASGYTFTSHWMHWVRQAPGQGLEWIGEFNPSNGRTNYNEKFKSKATMTVDTSTNTAYMELSSLRSEDTAVYYCASRDYDYAGRYFDYWGQGTLVTVSSA

SEQ ID NO: 854 - protein, 107 aa, synthetic construct
DIQMTQSPSSLSASVGDRVTITCSASSSVTYMYWYQQKPGKAPKLLIYDTSNLASGVPSRFSGSGSGTDYTFTISSLQPEDIATYYCQQWSSHIFTFGQGTKVEIKR

SEQ ID NO: 855 - protein, 121 aa, Synthetic construct
QVQLQQPGAELVKAGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSA

SEQ ID NO: 856 - protein, 106 aa, synthetic construct [sequence omitted]

(19) United States
(12) Patent Application Publication
(10) Pub. No.: US 2026/0102510 A1
(43) Pub. Date: Apr. 16, 2026

(54) CONDITIONING FOR IN VIVO IMMUNE CELL ENGINEERING

(71) Applicant: CAPSTAN THERAPEUTICS, INC., San Diego, CA (US)

(72) Inventors: Haig AGHAJANIAN, Lafayette Hill, PA (US); Adrian lon BOT, Beverly Hills, CA (US)

(21) Appl. No.: 19/103,974

(22) PCT Filed: Aug. 17, 2023
(86) PCT No.: PCT/US2023/072426

§ 371 Date: Feb. 14, 2025

Related U.S. Application Data
(60) Provisional application No. 63/371,742, filed on Aug. 17, 2022.
(60) Provisional application No. 63/382,397, filed on Nov. 4, 2022.
(60) Provisional application No. 63/510,610, filed on Jun. 27, 2023.

(51) Int. Cl. A61K 48/00; A61K 38/17; A61K 38/19; A61K 38/20; A61K 39/00; A61K 39/395; A61K 40/11; A61K 40/30

(52) U.S. Cl. CPC A61K 48/0041; A61K 38/1793; A61K 38/195; A61K 38/208; A61K 38/2086; A61K 39/39541; A61K 40/11; A61K 40/30; A61K 48/0058; A61K 2039/53; A61K 2039/55511; A61K 2039/6093

(57) ABSTRACT

Disclosed are methods of conditioning subjects who receive, are receiving, or have received an agent for in vivo reprogramming of immune cells in order to improve the efficiency of the in vivo reprogramming and/or the overall therapeutic effect of the treatment. Also disclosed are nanoparticle compositions for providing the conditioning agent(s). The conditioning agent can be provided prior to, concurrently with, or after administration of the in vivo reprogramming agent depending on the conditioning agent. The conditioning regimens are useful in combination with in vivo reprogramming of immune cells to treat hematologic cancers and solid tumor, fibrotic disorders, and B cell or T cell mediated autoimmunity, chronic infection. Some conditioning regimens are also useful in combination with other cancer treatments.

### CROSS-REFERENCE TO RELATED APPLICATIONS

This application claims the benefit of U.S. Provisional Patent Application No. 63/371,742, filed on Aug. 17, 2022; U.S. Provisional Patent Application No. 63/382,397, filed on Nov. 4, 2022; and U.S. Provisional Patent Application No. 63/510,610, filed on Jun. 27, 2023. The contents of each of these provisional applications are incorporated by reference in their entirety.

### SEQUENCE LISTING

This application contains a ST.26 compliant Sequence Listing, which was submitted in XML format via Patent Center, and is hereby incorporated by reference in its entirety. The XML copy, created on Aug. 17, 2023, is named 1467588007WO01.xml and is 969,000 bytes in size.

### BACKGROUND

Clinical practice of immune cell engineering currently exists in the form of chimeric antigen receptor (CAR)-T cell therapy. In broad terms, T cells are isolated from the blood by apheresis, engineered typically with a viral vector to express a CAR, expanded, and infused into the patient. Prior to infusion of the engineered cells the patient will undergo conditioning with lymphodepleting chemotherapy, which greatly improves efficacy of the CAR-T therapy. Lymphodepleting chemotherapy has multiple effects on the immune system and tumor microenvironment, likely including elimination of sinks for homeostatic cytokines such as IL-2, IL-7, and IL-15, eradication of immunosuppressive T regulatory cells (Tregs) and myeloid-derived suppressor cells, induction of co-stimulatory molecules, and downregulation of indoleamine 2,3-dioxygenase (an immune checkpoint molecule) in tumor cells, resulting in promotion of expansion, function, and persistence of the CAR-T cells once infused. However, for immunotherapies involving in vivo engineering of immune cells, lymphodepleting chemotherapy is not an appropriate conditioning regimen as it would tend to eliminate the very cells sought to be engineered.

Therefore, this disclosure provides conditioning regimens compatible with in vivo immune cell engineering to satisfy an urgent need in the field.

### SUMMARY

In one aspect, disclosed herein is a method of conditioning a subject who is to receive, is receiving, or has received an agent to engineer an immune cell in vivo, the conditioning comprising administration of a conditioning agent. In some embodiments, the conditioning agent is a biological response modifier (BRM). In some embodiments, the conditioning agent is low-dose cyclophosphamide. In some embodiments, the method comprises administration of at least one dose of the in vivo engineering agent. In some embodiments, the subject is one who receives, is receiving, or has received at least one dose of the in vivo engineering agent. In some embodiments, the BRM is administered as pre-treatment conditioning. In some embodiments, the BRM is administered as concurrent conditioning. In some embodiments, the BRM is administered after the in vivo engineering agent, as adjuvant conditioning. Adjuvant conditioning can also be administered concurrently. In some embodiments, the BRM is administered systemically. In some embodiments, the BRM is administered preferentially to a tumor or other diseased tissue or cells that could be beneficially reduced or eliminated by, for example, CAR-T or TCR-therapy. Examples of such other conditions include regenerative medicine-related conditions where treatment can lead to resolution of fibrosis and autoimmunity. In various instances, the BRM is systemically administered, or administered in a targeted nanoparticle, in a tropic nanoparticle (in which preferential uptake by tumor or other diseased tissue arises from the composition of the nanoparticle with the benefit of a specific binding moiety), or by local injection (such as intratumoral injection or, in the instance of ovarian cancer, intraperitoneal injection) or topical application.

Certain aspects include an activating conditioning regimen for expanding the number of polyfunctional immune effector cells or mobilizing immune effector cells comprising providing an activating conditioning agent prior to or concurrently with an in vivo immune cell engineering agent (see FIG. 1), wherein the activating conditioning agent comprises a γ-chain receptor cytokine, an inflammatory chemokine, a pan-activating cytokine, or a CTLA-4 checkpoint inhibitor. In some embodiments, the activating conditioning agent is provided by administering the activating conditioning agent. In some embodiments, the activating conditioning agent is provided by administering a nanoparticle (NP) comprising a nucleic acid encoding the activating conditioning agent. In this and other activating conditioning aspects, some embodiments are methods of conditioning or preparing or priming a subject to receive an in vivo immune cell engineering agent.

Certain aspects include an adjuvant conditioning regimen for diminishing Treg cell activity or recruiting endogenous immunity comprising providing an adjuvant conditioning agent concurrently with or after administration of an in vivo engineering agent (see FIG. 1), wherein the adjuvant conditioning agent comprises an immune checkpoint inhibitor, a low-dose cyclophosphamide, a γ-chain receptor cytokine, an antigen presenting cell activity enhancer, or a pan-activating cytokine. In some embodiments, the adjuvant conditioning agent is provided by administering the adjuvant conditioning agent. In some embodiments, the adjuvant conditioning agent is provided by administering a nanoparticle comprising a nucleic acid encoding the adjuvant conditioning agent. In this and other adjuvant conditioning aspects, some embodiments are methods of conditioning a subject who has received an in vivo immune cell engineering agent.

Certain aspects include a pre-treatment activating conditioning regimen for priming the immune system prior to in vivo reprogramming comprising systemic administration of a γ-chain receptor cytokine. The γ-chain receptor cytokine is delivered in one or multiple doses prior to administration of an in vivo engineering agent (see FIG. 1). In some embodiments, three weekly administrations of the γ-chain receptor cytokine are made with the final administration three to seven days before scheduled (or actual) administration of the in vivo engineering agent. In various embodiments, the γ-chain receptor cytokine comprises IL-15, IL-2, IL-7, or IL-21 delivered as recombinant protein. In some embodiments, the subject is administered at least an initial dose of the in vivo engineering agent. In some embodiments, the subject is one who receives at least one dose of an in vivo engineering agent. In some embodiments, the subject is one who receives at least one dose of an in vivo engineering agent after having received a last dose of the γ-chain cytokine three to seven days previously. Pre-treatment conditioning with γ-chain cytokines can also be used in combination with a variety of other cancer therapies including other immunotherapies (such as immune checkpoint inhibition therapy or anti-tumor antigen mAb therapy), targeted therapies (such as with kinase inhibitors), chemotherapies, radiotherapies, or cell-based therapies (such as adoptive transfer of CAR- or TCR-modified immune cells, tumor infiltrating lymphocytes (TIL), monocytes, or macrophages). This pre-treatment regimen can also be used in combination with treatments for autoimmune or fibrotic disorders.

Certain aspects include a pre-treatment activating conditioning regimen for priming the immune system prior to in vivo reprogramming comprising targeted or tropic administration of a nucleic acid encoding a γ-chain receptor cytokine. The γ-chain receptor cytokine-encoding nucleic acid is delivered in one or multiple doses prior to administration of an in vivo engineering agent or other therapy (see FIG. 1). In some embodiments, three weekly administrations of the γ-chain receptor cytokine-encoding nucleic acid are made with the final administration three to seven days before scheduled (or actual) administration of the in vivo engineering agent. The effect of the conditioning is finite in length so that with extended treatment with the in vivo engineering agent the treatment may be paused after one to three months, and the immune system reprimed with the conditioning agent. Multiple cycles of priming and reprogramming can be carried out for as long as a therapeutic benefit is expected or obtained. In various embodiments, the γ-chain receptor cytokine comprises IL-15, IL-2, IL-7, or IL-21. In some embodiments, the γ-chain receptor cytokine is provided as encoding mRNA packaged in a targeted or tropic nanoparticle. In other embodiments, the γ-chain receptor cytokine is provided encoded in a (non-mRNA) nucleic acid vector packaged in a targeted nanoparticle (tLNP) or tropic nanoparticle (trLNP). In some embodiments, the nanoparticle is a lipid nanoparticle. The targeted nanoparticle in which the γ-chain receptor cytokine is provided comprises a binding moiety for a tumor antigen expressed by the tumor to be treated. In some instances, the tumor antigen is a surface antigen on a neoplastic tumor cell while in other instances the tumor antigen is a surface antigen on a stromal tumor cell. In some embodiments, the subject is administered at least an initial dose of an in vivo engineering agent. In some embodiments, the subject is one who receives at least one dose of an in vivo engineering agent. In some embodiments, the subject is one who receives at least one dose of an in vivo engineering agent after having received a last dose of the γ-chain cytokine-encoding nucleic acid three to seven days previously. This pre-treatment conditioning regimen can be used in combination with a variety of cancer therapies including other immunotherapies (such as immune checkpoint inhibition therapy), targeted therapies (such a with kinase inhibitors), chemotherapies, radiotherapies, or cell-based therapies (such as adoptive transfer of CAR- or TCR-modified immune cells, tumor infiltrating lymphocytes (TIL), monocytes, or macrophages). This pre-treatment regimen can also be used in combination with treatments for autoimmune or fibrotic disorders.

Certain aspects include a conditioning regimen to facilitate in vivo reprograming of the immune system comprising systemic administration of an immune checkpoint inhibitor. In some embodiments, the immune checkpoint inhibitor comprises an anti-CTLA-4 antibody. In some embodiments, the immune checkpoint inhibitor comprises an anti-PD-1, anti-PD-L1, anti-Tim-3, or anti-LAG-3 antibody. Commonly, immune checkpoint inhibitors are administered by intravenous or subcutaneous infusion of the antibody (or other molecule), however, use of encoding nucleic acid vectors or mRNA are also possible. In some embodiments, the method comprises administration of at least one dose of an in vivo engineering agent. In some embodiments, the subject is one who receives or has received at least one dose of an in vivo engineering agent. Immune checkpoint inhibitor antibodies are often administered from one to two times per month (for example, every three weeks). In some embodiments, the immune checkpoint inhibitor is administered twice, three weeks apart. In various embodiments, such administration can precede, overlap, or follow administration of the in vivo engineering agent (see FIG. 1). In some embodiments, a second or greater administration of the immune checkpoint inhibitor takes place one week prior to a scheduled (or actual) initial administration of the in vivo engineering agent. In some embodiments, the final administration of the immune checkpoint inhibitor takes place the same day (plus or minus one day) as the initial administration of the in vivo engineering agent. In some embodiments, the final administration of the immune checkpoint inhibitor takes place two days to two weeks after the initial administration of the in vivo engineering agent. In some embodiments, a second dose of the immune checkpoint inhibitor is administered one week prior to the initial administration of the in vivo engineering agent and a third dose is administered two weeks after the initial administration of the in vivo engineering agent. In addition to oncologic treatment, this conditioning regimen can also be combined with treatments for autoimmune and fibrotic disorders.

Certain aspects include a conditioning regimen to facilitate in vivo reprograming of the immune system, or to augment other therapies, comprising targeted or tropic administration of a nucleic acid-encoded immune checkpoint inhibitor. In some embodiments, the immune checkpoint inhibitor comprises an anti-CTLA-4 antibody. In some embodiments, the immune checkpoint inhibitor comprises an anti-PD-1, anti-PD-L1, anti-Tim-3, or anti-LAG-3 antibody. In some embodiments, the immune checkpoint inhibitor is provided as encoding mRNA packaged in a targeted or tropic nanoparticle. In other embodiments, the immune checkpoint inhibitor is provided encoded in a (non-mRNA) nucleic acid vector packaged in a targeted or tropic nanoparticle. The targeted nanoparticle in which the encoded immune checkpoint inhibitor is provided comprises a binding moiety for a tumor antigen expressed by the tumor to be treated. In some instances, the tumor antigen is a surface antigen on a neoplastic tumor cell while in other instances the tumor antigen is a surface antigen on a stromal tumor cell.

The nanoparticle comprising the encoded immune checkpoint inhibitor can be administered by intravenous, intraperitoneal, or intralesional infusion or injection. The frequency of administration of the encoded immune checkpoint inhibitor depends in part on the proliferation rate of the targeted cells. When the targeted cells are proliferating slowly or not at all (as may be the case, for example, with some stromal cells), enough antibody can be produced locally so that the encoded immune checkpoint inhibitor can be administered on a similar schedule as the systemically administered immune checkpoint inhibitor itself. When the targeted cells are proliferating rapidly (as will be the case for neoplastic cells of an aggressive cancer), the mRNA will experience substantial dilution and turnover so that more frequent administration of the encoded immune checkpoint inhibitor can be required. Thus, in some embodiments, the encoded immune checkpoint inhibitor is administered one to two times per month (for example, every three weeks) while in other embodiments, the encoded immune checkpoint inhibitor is administered more frequently, for example, every three to four days over a period of one to three weeks.

This targeted immune checkpoint inhibitor conditioning regimen can be used in combination with a variety of cancer therapies including immunotherapies (such as CAR-, TCR-therapy), targeted therapies (such a with kinase inhibitors), chemotherapies, radiotherapy, or cell-based therapy (such as adoptive transfer of CAR- or TCR-modified immune cells, tumor infiltrating lymphocytes (TIL), monocytes, or macrophages) and can be practiced before the treatment, concurrently with the treatment, following the treatment, or some combination thereof. In some embodiments, the method comprises administration of at least one dose of an in vivo engineering agent or other treatment. In some embodiments, the subject is one who receives or has received at least one dose of an in vivo engineering agent or other treatment. In general, the targeted or tropic encoded immune checkpoint inhibitor can be administered on the same schedules, and in combination with the same treatments, as described for the immune checkpoint inhibitor antibodies above.

Certain aspects include a conditioning regimen to facilitate in vivo reprograming of the immune system, or to augment other therapies, comprising targeted administration of inflammatory chemokines. The targeted nanoparticle in which the inflammatory chemokines is provided comprises a binding moiety for a tumor antigen expressed by the tumor to be treated. In some instances, the tumor antigen is a surface antigen on a neoplastic tumor cell while in other instances the tumor antigen is a surface antigen on a stromal tumor cell. In some embodiments, the inflammatory chemokines comprise CCL5. In some embodiments, the inflammatory chemokine comprises CXL9, CXL10, or CXL11. In some embodiments, the chemokine is provided as encoding mRNA packaged in a targeted or tropic nanoparticle. In other embodiments, the chemokine is provided encoded in a (non-mRNA) nucleic acid vector packaged in a targeted or tropic nanoparticle. The targeted nanoparticle in which the chemokine is provided comprises a binding moiety for a tumor antigen expressed by the tumor to be treated. The nanoparticle can be administered by intravenous, intraperitoneal, or intralesional infusion or injection, for example, every three to four days. In some embodiments, the method comprises administration of at least one dose of an in vivo engineering agent. In some embodiments, the subject is one who receives at least one dose of an in vivo engineering agent. In some embodiments, the subject is one who receives at least one dose of an in vivo engineering agent after having received an mRNA encoding an inflammatory chemokine packaged in a targeted nanoparticle one week before.

Certain aspects include a conditioning regimen to facilitate in vivo reprograming of the immune system, or to augment other therapies, comprising systemic or targeted administration of an agent that enhances the activity of antigen presenting cells. In some embodiments, the agent that enhances the activity of antigen presenting cells comprises Flt3 ligand. In some embodiments, the agent that enhances the activity of antigen presenting cells comprises gm-CSF, or IL-18. The agent or mRNA encoding the agent is packaged in a nanoparticle targeted to or has tropism for a tumor cell. The targeted nanoparticle in which the agent that enhances the activity of antigen presenting cells is provided comprises a binding moiety for a tumor antigen expressed by the tumor to be treated. In some instances, the tumor antigen is a surface antigen on a neoplastic tumor cell while in other instances the tumor antigen is a surface antigen on a stromal tumor cell. The agent that enhances the activity of antigen presenting cells can be administered prior to, concurrently with, or subsequent to administration of an in vivo engineering agent (see FIG. 1). Thus, in some embodiments, the subject has received an in vivo engineering agent prior to administration of the antigen presentation enhancing agent. In some embodiments, the subject is receiving an in vivo engineering agent concurrently with administration of the antigen presentation enhancing agent (“concurrently with” can indicate on the same day as a single administration of the in vivo engineering agent or within the interval of time in which multiple administrations of the in vivo engineering agent are received). In some embodiments, the subject is one who receives the in vivo engineering agent after the antigen presentation enhancing agent has been administered. In some embodiments, the nanoparticles in which the antigen presentation enhancing agent or encoding mRNA is packaged is administered intravenously, while in other embodiments the administration is intraperitoneal or intralesional.

Certain aspects include a conditioning regimen to facilitate in vivo reprograming of the immune system, or to augment other therapies, comprising targeted administration of a highly active BRM enhancing the activity of all arms of the cellular immune system (e.g., a pan-activating cytokine). In some embodiments, the BRM is a cytokine that has dose-limiting toxicity if administered systemically. In some embodiments, the highly active BRM is IL-12. In some embodiments, the highly active BRM is IL-18. In some embodiments, the highly active BRM is provided as encoding mRNA packaged in a targeted or tropic nanoparticle. In other embodiments, the highly active BRM is provided encoded in a (non-mRNA) nucleic acid vector packaged in a targeted or tropic nanoparticle. The targeted nanoparticle in which the highly active BRM is provided comprises a binding moiety for a tumor antigen expressed by the tumor to be treated. In some instances, the tumor antigen is a surface antigen on a neoplastic tumor cell while in other instances the tumor antigen is a surface antigen on a stromal tumor cell. In other embodiments, the highly active BRM or mRNA encoding the highly active BRM is packaged in a nanoparticle having tropism for the tumor cell. To further limit systemic toxicity, in some embodiments, to inhibit uptake by untargeted cells the nanoparticle has CD47 or an effective portion thereof anchored to its surface. To further limit systemic toxicity, in some embodiments, the mRNA packaged in the nanoparticle contains a miRNA target domain to inhibit expression in non-target cells. For example, miRNA 122 will suppress translation of an mRNA containing its target domain in liver cells. The nanoparticle can be administered by intravenous, intraperitoneal, or intralesional infusion or injection. In some embodiments, the highly active BRM is administered prior to the subject receiving an in vivo engineering agent. In some embodiments, the highly active BRM is administered to a subject who has previously received an in vivo engineering agent (see FIG. 1). In some embodiments, the method comprises administration of at least one dose of an in vivo engineering agent. In some embodiments, the subject is one who receives or has received at least one dose of an in vivo engineering agent. In some embodiments, the subject is one who receives at least one dose of an in vivo engineering agent after having received a highly active BRM or encoding mRNA packaged in a targeted nanoparticle three to seven days before.

Certain aspects include a conditioning regimen to facilitate in vivo reprograming of the immune system comprising administration of low dose cyclophosphamide. Prior to administration of an in vivo immune engineering agent, a subject who is to receive an in vivo engineering agent is administered metronomic cyclophosphamide, for example 50 mg daily or 100 mg every other day. In some embodiments, the cyclophosphamide is administered over a period of five to eight days. In some embodiments, the method comprises administration of at least one dose of an in vivo engineering agent. In some embodiments, the subject is one who receives at least one dose of an in vivo engineering agent. In some embodiments, the subject is one who receives at least one dose of an in vivo engineering agent after having received a final dose of the cyclophosphamide three to four days previously.

It can also be advantageous to combine two or more of the above aspects in the treatment of an individual. Thus, an activating conditioning regimen and an adjuvant conditioning regimen can both be used in a combined treatment. Similarly, plural activating conditioning regimens utilizing different conditioning agents or plural adjuvant conditioning regiments utilizing different conditioning agents can be used in a combined treatment.

Many aspects are methods of conditioning practiced upon a subject who receives an engineering agent, however, there are parallel method of treatment aspects including an active step of administering the engineering agent.

In one aspect, disclosed herein is a composition comprising a targeted nanoparticle bearing a binding moiety on its surface to target the nanoparticle to a tumor or other diseased tissue and comprising a biological response modifier or a nucleic acid encoding the biological response modifier. In some embodiments, the nanoparticle is a lipid nanoparticle. In some embodiments, the binding moiety comprises an antibody or antigen binding portion thereof. In some embodiments, the binding moiety binds to a tumor antigen expressed on the surface of a tumor cell. In some instances, the tumor cell is a neoplastic cell. In some instances, the tumor cell is a stromal cell. In various embodiments, the BRM comprises a γ-chain receptor cytokine or agonist, an immune checkpoint inhibitor, an inflammatory chemokine, an enhancer of APC activity, or a highly active cytokine.

In another aspect, disclosed herein is a method of making a tLNP, comprising rapid mixing of an aqueous solution of a nucleic acid encoding a BRM and an alcoholic solution of lipids. In some embodiments, the lipid mixture includes functionalized PEG-lipid, for later conjugation to a targeting moiety. In other embodiments, the functionalized PEG-lipid is inserted into an LNP subsequent to initial formation of an LNP from other components. In either type of embodiment, the targeting moiety is conjugated to functionalized PEG-lipid after the functionalized PEG-lipid containing LNP is formed. Protocols for conjugation can be found, for example, in Parhiz et al., J. Controlled Release 291:106-115 (2018) and Tombacz et al., Molecular Therapy 29(11):3293-3304 (2021), each of which is incorporated by reference for all that it teaches about conjugation of PEG-lipids to binding moieties that is not inconsistent with the present disclosure.

### BRIEF DESCRIPTION OF THE DRAWINGS

FIG. 1 depicts a general example of the relative timing and effects of conditioning treatments for in vivo reprogramming of the immune system. A treatment with plural administrations of a tLNP (grey block arrows) for in vivo engineering of immune cells to express a CAR or TCR is shown. This in vivo engineering agent induces transient expression of the CAR or TCR by targeted immune cells (such as T cells; thick black curves). It is scalable and amenable to multiplexing. This contrasts with the permanent expression of the CAR or TCR in current ex vivo engineering of immune cells, such as in marketed CAR-T therapies. Plural administrations of an activating conditioning agent (white block arrows) are shown prior to administration of the in vivo engineering agent, increasing the number of polyfunctional immune cells (such as T cells; thin black curve) available to be reprogrammed. This contrasts with the immunodepleting conditioning carried out prior to current ex vivo engineering of immune cells, such as in marketed CAR-T therapies. Plural administrations of an adjuvant conditioning agent (black block arrows) are shown subsequent to administration of the in vivo engineering agent. The adjuvant conditioning can increase the level of activity of the reprogrammed cells through the suppression of regulatory T cell (Treg) activity or the inhibition of immune checkpoints. This also can increase endogenous anti-tumor activity through those same mechanisms, through recruitment, and through the promotion of epitope spreading (dotted and dashed black curve). The diagonal dotted lines extending from one of the activating conditioning block arrows and one of the adjuvant conditioning block arrows are present to indicate the effect of activating conditioning on the number of polyfunctional T cells generally and of adjuvant conditioning on endogenous immunity generally (and not specifically tied to the particular individual administration). Particular embodiments make use of either activating or adjuvant conditioning or both, using one or more conditioning agents of each type.

FIG. 2 depicts a general example of tLNP transfection where the targeting antibody on the tLNP binds to the target on the surface of the target T cell. After binding, the tLNP is endocytosed, the tLNP escapes the endosome with degradation of the tLNP and release of mRNA into the cytoplasm. The mRNA is then translated, and the protein expressed.

FIGS. 3A-3J show that IL-7 pre-treatment improves transfection efficacy of CD5-mCherry tLNPs in vitro. FIG. 3A illustrates experimental design for in vitro T cell activation in embodiments of the present technology. T cells were magnetically isolated from the spleens of C57BL/6 mice and activated with αCD3/CD28 beads and supplemented with IL-2. Forty-eight hours later, beads were removed and 1 μg of CD5-targeted tLNPs were added per million cells and flow cytometry was performed 24 hours later. FIG. 3B shows representative flow plots of non-activated (media; upper two panels) and activated T cells (lower two panels) untreated (left two panels) and treated (right two panels) with mCherry tLNPs. FIG. 3C illustrates experimental design for testing LNP targeting in vivo in particular embodiments of the present technology. Ten μg of IgG-mCherry or CD5-mCherry targeted LNPs were administered intravenously to mice and flow cytometry was performed on spleen and lymph nodes that were collected 24 hours after treatment. FIGS. 3D-3E show the percent mCherry⁺ CD4⁺ (FIG. 3D) or mCherry⁺ CD8⁺ (FIG. 3E) T cells in the mouse spleen. FIGS. 3F-3G show the percent mCherry⁺ CD4⁺ (FIG. 3F) or mCherry⁺ CD8⁺ (FIG. 3G) T cells in the mouse lymph node. FIG. 3H shows the experimental design for in vitro cytokine treatment of T cells isolated from spleens in particular embodiments of the present technology. T cells from mice were isolated and cultured with either IL-2, IL-7, or IL-15. Cytokines were refreshed daily and tLNPs added on day 2. Flow cytometry was performed on the cells on day 3. FIGS. 3I-3J show the percent of mCherry⁺ CD4⁺ (FIG. 3I) and mCherry⁺ CD8⁺ (FIG. 3J) T cells in vitro after the cells were treated with IL-2, IL-7, or IL-15. Media treated and αCD3/CD28 bead-activated T cells were used as controls. Treated cells were compared to the media-only control using a one-way ANOVA with Sidak's test, which was used for multiple comparisons. *p<0.05, **p<0.01, ***p<0.001, ****p<0.0001

FIGS. 4A-4I show that IL-7 enhances CD5-mCherry tLNP transfection efficiency in vivo. FIG. 4A illustrates experimental design for T cell activation and tLNP targeting after treatment with IL7 in vivo in particular embodiments of the present technology. C57BL/5 mice were injected interperitoneally with 5 μg of recombinant murine IL-7 daily for three days. On the third day mice received 10 μg CD5-mCherry tLNPs intravenously. Twenty-four hours after tLNP treatment spleens and lymph nodes were collected for flow cytometry. FIGS. 4B-4C show the percent of mCherry⁺ CD4⁺ (FIG. 4B) and mCherry⁺ CD8⁺ (FIG. 4C) T cells in the spleen. FIGS. 4D-4E show the proportion of mCherry⁺ CD4⁺ (FIG. 4D) and mCherry⁺ CD8⁺ (FIG. 4E) T cells in lymph nodes. FIGS. 4F-4G show the total number of mCherry⁺ CD4⁺ (FIG. 4F) and mCherry⁺ CD8⁺ (FIG. 4G) T cells in the spleen. FIGS. 4H-4I show the total number of mCherry⁺ CD4⁺ (FIG. 4H) and mCherry⁺ CD8⁺ (FIG. 4I) T cells in the lymph node. One-way ANOVA with Sidak's test was used for multiple comparisons. *p<0.05, **p<0.01, ***p<0.001, ****p<0.0001.

FIGS. 5A-5F show that IL-7 treated CD8⁺ T cells are enriched for translation and metabolism associated pathways. FIG. 5A illustrates experimental design for performing RNA sequencing on IL-7 treated T cells in particular embodiments of the present technology. CD8⁺ T cells were isolated from the spleens of C57BL/6 mice and cultured with T cells media alone or supplemented with IL-2, IL-7, or IL-15. After 48 hours T cells were collected, and bulk RNA sequencing was performed. FIG. 5B shows variance stabilizing transformation (VST)-normalized principal component analysis of T cells treated with each cytokine. FIG. 5C shows the volcano plot showing the differentially expressed genes between IL-7 and IL-15 treated CD8 T cells. Genes with a positive log 2 fold change are upregulated with IL-7 treatment compared to IL-15, while a negative log 2 fold change indicates upregulation with IL-15 treatment compared to IL-7. FIGS. 5D-5F show gene set enrichment analysis using the list of differentially expressed genes between IL-7 and IL-15 treated cells using the hallmarks (FIG. 5D), reactome (FIG. 5E) or gene ontology biological processes (GOBP) (FIG. 5F) databases. Gene sets associated with translation and metabolism are shown from the GOBP analysis. Size of point indicates the false discovery rate (FDR)(log 10padj) with a positive net enrichment score (NES) indicating enrichment in IL-7 treated cells and a negative NES indicating enrichment in IL-15 treated cells.

FIGS. 6A-6C show that IL-7 increases the translation of mRNA in T cells in vitro. FIG. 6A illustrates experimental design for testing the effect of cytokine treatment on electroporation of T cells in particular embodiments of the present technology. T cells were isolated from the spleen C57BL/6 mice and either activated using CD3/CD28 beads or cultured in T cells media supplemented with IL-2, IL-7, or IL-15. After 48 hours, T cells were electroporated with 2 μg of mCherry mRNA per 1 million cells. mCherry expression was measured 24 hours later. FIGS. 6B-6C show the proportion of mCherry⁺ CD4+(FIG. 6B) or mCherry⁺ CD8+(FIG. 6C) T cells after electroporation with mCherry mRNA. One-way ANOVA with Sidak's test was used for multiple comparisons. *p<0.05, **p<0.01. **p<0.001, ****p<0.0001.

FIGS. 7A-7G show that IL-7 pre-treatment of human T cells improves the transfection efficiency of CD5-mCherry tLNPs in vitro. FIG. 7A illustrates experimental design for tLNP transfection in particular embodiments of the technology. T cells were isolated from PBMC and either activated using anti-CD3/CD28 beads+100 IU/ml rhIL-2 or cultured in T cells media supplemented with IL-2, IL-7, or IL-15 and replenished after 48 hours. After 72 hours, T cells were transfected with 0.6 μg of CD5-LNP-mCherry per 2×10⁵ cells. mCherry expression was measured by flow cytometry 24 hours later. The data in FIGS. 7B-7D were generated using tLNPs incorporating ALC-0315 as the ionizable cationic lipid and the data in FIGS. 7E-7G were generated using tLNPs incorporating CICL1 as the ionizable cationic lipid.

FIGS. 7B-7C and 7E-7F show the percent of mCherry⁺ CD4+(FIGS. 7B and 7E) or mCherry⁺ CD8+(FIGS. 7C and 7F) T cells after transfection with mCherry mRNA.

FIGS. 7D and 7G shows representative flow cytometry plots of rested (media; upper two panels) and IL-7-cultured T cells (lower two panels), untransfected (left two panels) or transfected (right two panels) with CD5-LNP mCherry. One-way ANOVA with Sidak's test was used for multiple comparisons. *p<0.05, **p<0.01, ***p<0.001, ****p<0.0001. N=3 from two separate healthy human donors.

FIG. 8 shows a schematic of an example use of IL-7 to enhance ex vivo tLNP transfection efficiency in particular embodiments of the technology. tLNPs may be generated utilizing IL-7 pre-treatment according to two methods. In one method, isolated T cells are cultured and expanded in media containing IL-7. tLNPs are added to transfect T cells and the product infused into the patient. In another method, conventionally generated CAR T cells are cultured in media containing IL-7. These can then be dual transfected by adding tLNPs to the CAR-T culture and then infused into the patient.

FIGS. 9A-9B show mCherry expression level (as geometric mean fluorescence intensity; FIG. 9A) and % transfected (FIG. 9B) following transfection of various T and B cell tumor cell lines and primary cells with tLNP targeted to CD19 (47G4 (CD19[a]) and FMC63 (CD19[b])), CD20 (2.1.2 (CD20[a]) and Leu16 CD20[b]), EGFR (cetuximab), HIV gp120 (teropavimab), CD5 (h5D7), and CD8 (chRPA-T8).

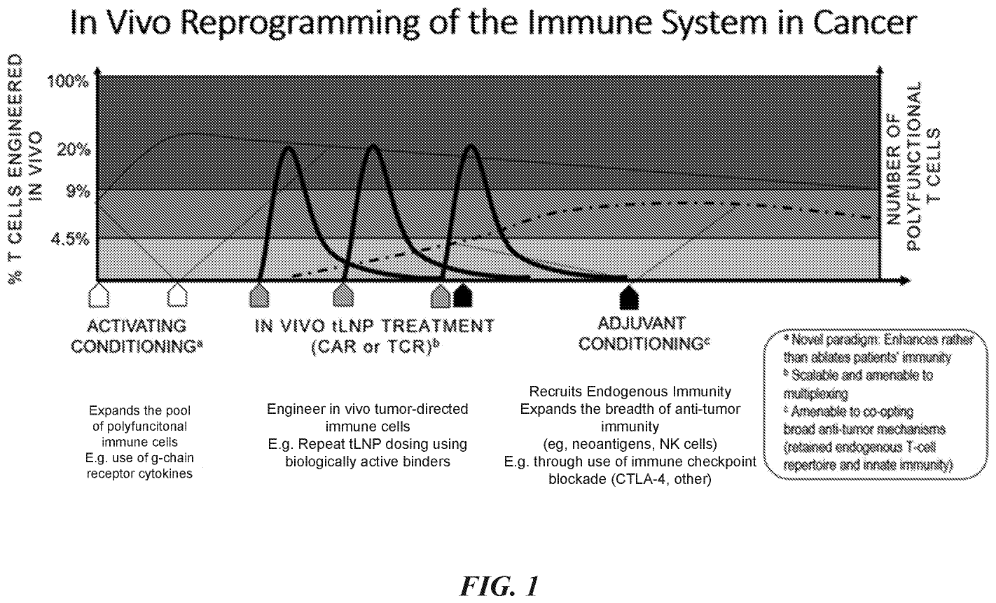

### DETAILED DESCRIPTION

Conditioning of immune cells to be more responsive to in vivo engineering as compared to unconditioned immune cells may be carried out in a variety of broad modes. In some embodiments, a conditioning agent is administered systemically, generally involving administration of the agent itself. In some aspects, a conditioning agent is an exogenous protein, for example, a recombinant protein that is administered systemically.

Alternatively, a conditioning agent can be delivered with a targeted or tropic administration. In some embodiments, targeted administration comprises administration of an encoding mRNA packaged in a nanoparticle bearing a binding moiety on its surface that will target the nanoparticle to a tumor or other diseased tissue. In tropic administration the composition of the nanoparticle itself leads to preferential uptake by the tumor or other diseased tissue without the benefit of a specific binding moiety. However, in some embodiments, a conditioning agent is encoded in DNA and is expressed episomally or after being integrated into the genome of the targeted cell. Integration can be accomplished by including an RNA-guided nuclease or an mRNA encoded RNA-guided nuclease, and a guide RNA in the nanoparticle in order to knock-in the DNA encoding a conditioning agent. In still other embodiments, local administration, such as intratumoral injection, intraperitoneal injection (for ovarian cancer), or topical application, is used to deliver a conditioning agent to the diseased tissue.

In targeted or tropic administration of a conditioning agent, the nanoparticle is often directed to the diseased cell or tissue; that is, to a tumor cell, an autoimmune effector cell, a fibrogenic cell or the affected tissue or organ.

While the present disclosure is capable of being embodied in various forms, the description below of several embodiments is made with the understanding that the present disclosure is to be considered as an exemplification of the invention and is not intended to limit the invention to the specific embodiments illustrated.

Headings are provided for convenience only and are not to be construed to limit the invention in any manner. Embodiments illustrated under any heading may be combined with embodiments illustrated under any other heading.

To the extent any materials incorporated herein by reference conflict with the present disclosure, the present disclosure controls.

Prior to setting forth this disclosure in more detail, it may be helpful to provide abbreviations and definitions of certain terms to be used herein. Additional definitions are set forth throughout this disclosure.

### Definitions

As used in the specification and claims, the singular form “a,” “an,” and “the” includes plural references unless the context clearly dictates otherwise. It should be understood that the terms “a” and “an” as used herein refer to “one or more” of the enumerated components.

The use of the alternative (e.g., “or”) should be understood to mean either one, both, or any combination thereof of the alternatives.

The term “about” as used herein in the context of a number refers to a range centered on that number and spanning 10% less than that number and 10% more than that number. The term “about” used in the context of a range refers to an extended range spanning 10% less than that the lowest number listed in the range and 10% more than the greatest number listed in the range.

Throughout this disclosure, any concentration range, percentage range, ratio range, or integer range is to be understood to include the value of any integer within the recited range and, when appropriate, fractions thereof (such as one tenth and one hundredth of an integer), unless otherwise indicated. Also, any number range of this disclosure relating to any physical feature, such as polymer subunits, size, or thickness, are to be understood to include any integer within the recited range, unless otherwise indicated. Throughout this disclosure, numerical ranges are inclusive of their recited endpoints, unless specifically stated otherwise.

Unless the context requires otherwise, throughout the present specification and claims, the word “comprise” and variations thereof, such as, “comprises” and “comprising” are to be construed in an open, inclusive sense, that is, as “including, but not limited to.” As used herein, the terms “include” and “comprise” are used synonymously.

The phrase “at least one of” when followed by a list of items or elements refers to an open-ended set of one or more of the elements in the list, which may, but does not necessarily, include more than one of the elements.

“Derivative,” as used herein, refers to a chemically or biologically modified version of a compound that is structurally similar to a parent compound and (actually or theoretically) derivable from that parent compound. Generally, a “derivative” differs from an “analogue” in that a parent compound may be the starting material to generate a “derivative,” whereas the parent compound may not necessarily be used as the starting material to generate an “analogue.” A derivative may have different chemical or physical properties than the parent compound. For example, a derivative may be more hydrophilic or hydrophobic, or it may have altered reactivity as compared to the parent compound. Although a derivative can be obtained by physical (for example, biological or chemical) modification of the parent compound, a derivative can also be conceptually derived, for example, as when a protein sequence is designed based on one or more known sequences, an encoding nucleic acid is constructed, and the derived protein obtained by expression of the encoding nucleic acid.

The terms “treatment” “treating”, etc., refer to the medical management of a patient with the intent to cure, mitigate, ameliorate, stabilize, or prevent a disease, pathological condition, or disorder in a human subject or other animals. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder. Various embodiments may specifically include or exclude one or more of these modes of treatment.

Treatment activity includes the administration of the conditioning agents, adjuvant conditioning agents, engineering agents, in vivo engineering agents, or other medicaments, dosage forms, pharmaceutical compositions described herein. As used herein, a “medicament” includes any of the dosage forms of the present technology, including conditioning agents, adjuvant conditioning agents, engineering agents, and in vivo engineering agents. Treatment activity includes administration to a patient, especially according to the various methods of treatment disclosed herein, whether by a healthcare professional, the patient his/herself, or any other person. Treatment activities include the orders, instructions, and advice of healthcare professionals such as physicians, physician's assistants, nurse practitioners, and the like, that are then acted upon by any other person including other healthcare professionals or the patient him/herself. In some embodiments, the orders, instructions, and advice aspect of treatment activity can also include encouraging, inducing, or mandating that a particular medicament, or combination thereof, be chosen for treatment of a condition—and the medicament is actually used—by approving insurance coverage for the medicament, denying coverage for an alternative medicament, including the medicament on, or excluding an alternative medicament, from a drug formulary, or offering a financial incentive to use the medicament, as might be done by an insurance company or a pharmacy benefits management company, and the like. In some embodiments, treatment activity can also include encouraging, inducing, or mandating that a particular medicament be chosen for treatment of a condition—and the medicament is actually used—by a policy or practice standard as might be established by a hospital, clinic, health maintenance organization, medical practice or physicians' group, and the like. All such orders, instructions, and advice are to be seen as conditioning receipt of the benefit of the treatment on compliance with the instruction. In some instances, a financial benefit is also received by the patient for compliance with such orders, instructions, and advice. In some instances, a financial benefit is also received by the healthcare professional for compliance with such orders, instructions, and advice.

As used herein “expansion,” “expanding,” and the like refers to an increase in the number of cells, especially within a tumor or other locus of disease. This increase can be due to proliferation and/or differentiation of the expanding cell type but can also include in-migration of cells into the tumor or other locus of disease due to mobilization. Expansion can increase the number of immune cells amenable to reprogramming both in the immune system generally or in a tumor or other locus of disease.

As used herein an “exogenous protein” refers to a synthetic, recombinant, natural, or other peptide or protein that is not produced by a wild-type cell of that type or is expressed at a lower level in a wild-type cell than in a cell containing the exogenous polypeptide. In some embodiments, an exogenous peptide or protein is a peptide or protein encoded by a nucleic acid that was introduced into the cell, which nucleic acid is optionally not retained by the cell. In some embodiments, an exogenous peptide or protein is a peptide or protein that is administered to an organism.

As used herein “extracorporeal” is used in reference to cells, such as peripheral blood or bone marrow cells, harvested or extracted from the body and the manipulation or modification of those cells prior to their intended return (reinfusion). Manipulation and modification of cells generally relates to cell separation and washing procedures and exposure to activation agents (e.g., biological response modifiers (BRMs)) and transfection agents (e.g., LNPs, tLNPs), over a time interval of several hours, for example, less than 6 hours, less than 5 hours, less than 4 hours, less than 3 hours, less than 2 hours, or less than 1 hour; and in space to a single institution. Extracorporeal is used in contradistinction to ex vivo which, as used herein, includes more extensive manipulation including extended periods of cell culture and expansion, and/or refrigerated or cryogenic storage or shipment, over several days or longer.

As used herein “transfection” or “transfecting” refers to the introduction of nucleic acids into cells by non-viral methods. Transfection can be mediated by calcium phosphate, cationic polymers, magnetic beads, electroporation and lipid-based reagents. In preferred embodiments disclosed herein transfection is mediated by solid lipid nanoparticles (LNP) including targeted LNP (tLNP). The term transfection is used in distinction to transduction—transfer of genetic material from cell to cell or virus to cell—and transformation—the uptake of extracellular genetic material by the natural processes of a cell. As used herein, phrases such as “delivering a nucleic acid into a cell” are synonymous with transfection.

“Reprogramming,” as used herein with respect to immune cells, refers to changing the functionality of an immune cell with respect to antigenic specificity by causing expression of an exogenous T cell receptor (TCR), a chimeric antigen receptor (CAR), or an immune cell engager (“reprogramming agents”). Generally, T lymphocytes and NK cells could be reprogrammed with a TCR, a CAR, or an immune cell engager while only a CAR or an immune cell engager would be used in reprogramming monocytes. Reprogramming can be transient or durable depending on the nature of the engineering agent.

As used herein, “reprogramming agent” (or similar constructions such as an agent to reprogram an immune cell) refers to a protein which changes the function of the immune cell in which it is expressed. In many embodiments the reprogramming agent comprises an antigen receptor, such as a CAR, a TCR, or an immune cell engager (for example a BiTE (a bispecific T cell engager)). Unlike CARs and TCRs, BiTEs and other immune cell engagers are secreted molecules. BiTEs can effectively redirect T cells whether secreted from T cells or other immune cells that take up the in vivo engineering agent, whether due to co-targeting (for example, CD2-targeted nanoparticles will also target NK cells), designed targeting to non-T cells, or off-target delivery, such as to hepatocytes. The same principles apply to immune cell engagers that engage non-T cells, such as NK cells, monocytes, and macrophages. As soluble molecules, immune cells engagers can also be usefully expressed by tumor cells or other pathogenic cells instead of being expressed in the immune cell. The CAR, TCR, or immune cell engager will generally bind to an antigen found on a tumor, autoimmunity-mediating, or other pathogenic cell.

“Engineering agent,” (or similar constructions such as an agent to engineer an immune cell in vivo) as used herein, refers to agents used to modify (engineer) a cell of the immune system. In particular, the engineering agent can confer the expression of a reprogramming agent by an immune cell, particularly a non-B lymphocyte or monocyte. Engineering agents can include nucleic acids, including mRNA, that encode the reprogramming agent. In certain embodiments, an engineering agent is an mRNA encoding a CAR, TCR, or immune cell engager. This mRNA could be linear, or circular, modified using pseudouridine or any other type of modification that would ameliorate its immunotoxicological profile and/or increase efficacy. It could also be a self-replicating RNA. Engineering agents can also include nucleic acids that are or encode components of gene editing systems such as RNA-guided nucleases, guide RNA, and nucleic acid templates for knocking-in a reprogramming agent or knocking-out an endogenous antigen receptor. Gene editing systems comprise base-editors, prime-editors or gene-writers. RNA-guided nucleases include CRISPR nucleases such as Cas9, Cas12, Cas13, Cas3, CasMINI, Cas7-11, and CasX. For transient expression of a reprogramming agent, such as a CAR, an mRNA encoding the reprogramming agent can be used as the engineering agent. For durable expression of the reprogramming agent, such as an exogenous, modified, or corrected gene (and its gene product), the engineering agent can comprise mRNA-encoded RNA-directed nucleases, guide RNAs, nucleic acid templates and other components of gene/genome editing systems. Such engineering agents can also be referred to as a means for engineering an immune cell in vivo.

Examples of gene editing components that are encoded by the nucleic acid include, but are not limited to, an mRNA encoding an RNA-guided nuclease, a gene or base editing protein, a prime editing protein, a Gene Writer protein (e.g., a modified or modularized non-long terminal repeat (LTR) retrotransoposon), a retrotransposase, an RNA writer, a zinc finger nuclease (ZFN), a transcription activator-like effector nuclease (TALEN), a meganuclease, a transposase, a retrotransposon, a reverse transcriptase (e.g., M-MLV reverse transcriptase), a nickase or inactive nuclease (e.g., Cas9, nCas9, dCas9), a DNA recombinase, a CRISPR nuclease (e.g., Cas9, Cas12, Cas13, Cas3, CasMINI, Cas7-11, CasX), a DNA nickase, a Cas9 nickase (e.g., D10A or H840A), or any fusion or combination thereof. Other components include a guide RNA (gRNA), a single guide RNA (sgRNA), a prime editing guide RNA (pegRNA), a clustered regularly interspaced short palindromic repeat (CRISPR) RNA (crRNA), a trans-activating clustered regularly interspaced short palindromic repeat (CRISPR) RNA (tracrRNA), or a DNA molecule to be inserted or serve as a template for double-strand break (DSB) repair at a specific genomic locus.

“Immune cell,” as used herein, can refer to any cell of the immune system. However, particular aspects can exclude polymorphonuclear leukocytes and/or B cells, or be limited to non-B lymphocytes such as T cell and/or NK cells, or to monocytes such as dendritic cells and/or macrophages in their various forms.

As used herein, a BRM (or immunomodulator) is a substance that modifies an immune response. As used in conditioning regimens, the BRM will promote the immune response to diseased tissue (e.g., tumor tissue) or suppress or inhibit regulatory responses that would otherwise diminish or block the immune response to diseased tissue. Such BRMs include cytokines, chemokines, and immune checkpoint inhibitors. BRMs are typically receptor ligands. Cytokines and chemokines are generally receptor agonists, as are some immune checkpoint inhibitors, but some BRMs are antagonists or otherwise block receptor activity, most notably many immune checkpoint inhibitors. Consequently, the various BRMs referred to herein may be substituted with alternative compounds that are also ligands (agonists or antagonists, as appropriate) of the particular BRM's receptor. Such alternative compounds can include peptidomimetics and aptamers. BRMs can also be modified to alter properties such as half-life and biodistribution without disrupting their basic agonistic (or antagonistic) activity and thus in some embodiments, serve as alternative compounds to the herein indicated BRMs. Such modifications can include pegylation or incorporation into fusion proteins (for example, fusion to the Fc portion of an antibody). Accordingly, the various cytokines, chemokines, immune checkpoint inhibitors and other BRMs, together with such alternatives, constitute means for accomplishing the function(s) of the BRMs.

“Conditioning agent,” as used herein, refers to a biological response modifier (BRM) that enhances the efficiency of engineering the immune cell, expands the number of immune cells available to be engineered or the number of engineered cells in the target tissue (for example, a tumor, fibrotic tissue, or tissue undergoing autoimmune attack), promotes presence or activity of the engineered cell in the target tissue, or broadens the range of operative mechanisms contributing to a therapeutic immune reaction. A conditioning agent may be provided by delivering an exogenous BRM itself or as an encoding nucleic acid in a tLNP.

Conditioning may be defined by the timing of its administration in relation to administration of an engineering agent, such as pre-treatment conditioning, concurrent conditioning, and post-treatment conditioning. In pre-treatment conditioning, a conditioning agent is administered prior to administration of an engineering agent. In various embodiments, a conditioning agent is administered one to several times in the week prior to administration of an engineering agent. In some embodiments the last pre-conditioning administration is the day before or the day of administration of an engineering agent. Pre-treatment conditioning is typically an activating conditioning. Post-treatment conditioning takes place subsequent to at least an initial dose of the engineering agent and may not itself be initiated until after a final dose of the engineering agent in a cycle of a set number of multiple doses. While pre-treatment conditioning and post-treatment conditioning can take place outside of the time interval in which an engineering agent is administered, concurrent conditioning extends over the same time interval as that over which an engineering agent is administered. Indeed, in some embodiments, an engineering agent and a conditioning agent are packaged in the same nanoparticle. In other embodiments the conditioning and engineering agents are packaged in separate nanoparticles, or a conditioning agent is administered systemically.

Conditioning can also be classified according to its effect. Activating conditioning leads to the expansion of polyfunctional immune effector cells amenable to in vivo engineering and/or the mobilization of immune effector cells resulting in the localization in tumor or other disease-associated tissue. The γ-chain receptor cytokines promote both effects stimulating both proliferation and migration. Proliferation of immune effector cells will also be stimulated by the highly active, pan-activating cytokines IL-12 and IL-18. Mobilization will also be promoted by inflammatory chemokines and anti-CTLA-4 (an immune checkpoint inhibitor). Activating conditioning is generally carried out prior to administration of the in vivo engineering agent, although it can continue to be given concurrently, especially when the in vivo engineering agent is administered multiple times at intervals of several days. Repeated cycles of activating conditioning followed by treatment with the in vivo engineering agent can also be used.

In some embodiments of activating conditioning the percentage of T cells engineered is ≥4.5%. In other embodiments of activating conditioning the percentage of T cells engineered is ≥9%. In various instances of these embodiments, the percentage of engineered T cells is ≤50%, 40%, 30%, or 20%. Assessment of percentage of engineered T cells is based targeting of the tLNP. If the binding moiety of the tLNP targets pan T cells (for example, by targeting CD2, CD3, CD5 or CD7) then the percentage is of total T cells. If the binding moiety of the tLNP targets a subset of T cells (for example, by targeting CD4 or CD8, etc.) then the percentage is based on total T cells in the subset (for example total CD4+ T cells or total CD8+ T cells, etc.).

Adjuvant conditioning aims to improve the efficacy of treatment and can act through the engineered cells themselves or through the recruitment of other elements of the immune system. Depletion of Treg cells, for example using anti-CTLA-4, an anti CCR4, or low-dose cyclophosphamide, will promote the activity (and thus effectiveness) of both the in vivo engineered cells and any endogenous antigen-specific T cells. Immune checkpoint inhibition can also promote the activity of antigen-specific responses through a reduction in Treg activity. Adjuvant conditioning can also augment the effect of the in vivo engineered cells by the recruitment of innate immunity with IL-15 and inflammatory chemokines, by T cell activation with γ-chain receptor cytokines, IL-12, and IL-18, and by promotion of epitope spreading with anti-CTLA-4. Flow cytometry and immunohistochemistry can be used to detect changes in number and activity of these various cell types.

In adjuvant conditioning, a conditioning agent is administered concurrent with or subsequent to administration of an engineering agent. In some embodiments, the initial dose of the adjuvant conditioning agent is administered on the same day as a first dose of an engineering agent while in other embodiments the initial dose of the adjuvant conditioning agent is only administered one or more days, up to two weeks, after administration of a last dose of an engineering agent. In similar embodiments, the initial administration of the adjuvant conditioning agent is indexed to a second, third, . . . or any subsequent dose, including a last dose of an engineering agent. In some embodiments, an adjuvant conditioning agent can be administered periodically for several weeks (or months). As indicated, adjuvant conditioning is generally carried out after at least an initial dose of the in vivo engineering agent has been administered, although it can proceed concurrently, especially when the in vivo engineering agent is administered multiple times at intervals of several days. When inflammatory chemokines are used for adjuvant conditioning, concurrent use is preferred. Repeated cycles of treatment with the in vivo engineering agent joined or followed by adjuvant conditioning can also be used.

Accordingly, in one aspect, disclosed herein is a method of conditioning a subject who is to receive, is receiving, or has received an agent to engineer an immune cell in vivo, the conditioning comprising administration of a biological response modifier (BRM). In some embodiments, the method comprises administration of at least one dose of the engineering agent. In some embodiments, the BRM is administered as pre-treatment conditioning. In some embodiments, the BRM is administered as concurrent conditioning. In some embodiments, the BRM is administered as adjuvant conditioning. In some embodiments, the BRM is administered only after the engineering agent has been administered. In some embodiments, the BRM is administered systemically. In some embodiments, the BRM is delivered preferentially to a tumor or other diseased tissue. In various instances, the BRM is administered in a targeted nanoparticle, in a tropic nanoparticle, or by local injection or topical application. The conditioning regimens and agents disclosed herein are for the purpose of improving the efficiency of modification and the efficacy of immune response to the targeted cells or tissue in which the engineered immune cell participates.

CARs have become marketed products with established generic structure comprising a signal sequence followed by an antibody-derived antigen binding domain, often but not necessarily a single chain Fv (scFv), a transmembrane domain and intracellular sequences comprising one or more costimulatory domains and an intracellular signaling domain. The signal sequence can be derived from the antibody, a TCR, CD8 or other type 1 membrane proteins, preferably a protein expressed in a T or other immune cell. The transmembrane domain can be one associated with any of the potential intracellular domains or from another type 1 membrane protein, such as TCR α, β, or ζ chain, CD3ε, CD4, CD8, or CD28, among other possibilities known in the art. The transmembrane domain can further comprise a hinge region located between the antigen binding domain and the hydrophobic membrane-spanning region of the transmembrane domain. The intracellular signaling domain can be derived from the CD3ζ chain, FcγRIII, FcsRI, or an immunoreceptor tyrosine-based activation motif (ITAM) bearing cytoplasmic domain, among other possibilities known in the art. The costimulatory domain can be derived from CD28, 4-1 BB, or DAP12, among other possibilities known in the art. Examples of CARs are disclosed in U.S. Pat. No. 7,446,190 (anti-CD19), U.S. Pat. No. 10,287,350 (anti-CD19), US2021/0363245 (anti-CD19 and anti-CD20), U.S. Pat. No. 9,765,342 (anti-BCMA), U.S. Pat. No. 10,543,263 (anti-CD22), U.S. Pat. No. 10,426,797 (anti-CD33), U.S. Pat. No. 10,844,128 (anti-CD123), U.S. Pat. No. 9,272,002 (anti-mesothelin), WO2022086620A1 and WO2023086336A2 (anti-PSMA), WO2021050656A1 (anti-PSCA), U.S. Pat. No. 10,428,141 (anti-ROR1), and US2021/0087295 and WO2022081694A1 (anti-FAP), each of which is incorporated by reference for all that it teaches about CAR structure and function generically and with respect to the CAR's antigenic specificity to the extent that it is not inconsistent with the present disclosure.

Further examples of CARs include those incorporating a CD19 binding moiety derived from the human antibody 47G4 or the mouse antibody FMC63. FMC63 and the derived scFv have been described in Nicholson et al., Mol. Immun. 34(16-17):1157-1165 (1997) and PCT Application Publication Nos. WO2018213337 and WO2015187528, the entire contents of each of which are incorporated by reference herein for all that they teach about anti-CD19 CARs and their use. CAR based on 47G4 are disclosed in U.S. Pat. No. 10,287,350 which is incorporated by reference herein for all that it teaches about anti-CD19 CARs and their use. In some instances, an anti-CD19 CAR is the CAR found in tisagenlecleucel, lisocabtagene maraleucel, axicabtagene ciloleucel, or brexucabtagene autoleucel.

In some embodiments, an engineering agent is a nucleic acid that encodes an immune cell engager, and the reprogramming agent is the immune cell engager, including, for example, a T cell engager (e.g., BiTE, DART), an NK cell engager (e.g., BiKE, TriKE), a macrophage engager (e.g., BiME), and an innate cell engager (e.g., ICE). BiTEs are a class of artificial bispecific monoclonal antibodies that direct a host's immune system, more specifically the T cells' cytotoxic activity, against cells bearing target antigens (e.g., cancer cells). BiTEs usually are fusion proteins having two single-chain variable fragments (scFvs) of different antibodies in a single peptide chain. One of the scFvs binds a T cell via the CD3 receptor, and the other to a target cell (e.g., a cancer cell) via a target cell-specific antigen. Unlike CARs and TCRs, BiTEs are secreted molecules. BiTEs can effectively redirect T cells whether secreted from T cells or other cells, whether due to co-targeting, designed targeting to non-T cells, or off-target delivery (such as to hepatocytes). Further immune cell engagers can be constructed by replacing the anti-CD3 moiety with a binding moiety specific for another immune cell surface molecule as disclosed herein to engage a different segment of the immune system. For example, using an anti-CD8 binding moiety instead of anti-CD3 in a BiTE-like protein would generate an immune cell engager limited to engaging just the CD8+ subset of T cells while using an anti-CD2 binding moiety would lead to engagement of both T cells and NK cells. A DART is a heterodimer of two scFv-like polypeptides, one containing the VL of a first antibody and the VH of a second antibody, and the other containing the VH of the first antibody and the VL of the second antibody, with the VH and VL regions within each chain connected by a short diabody-like linker to promote interchain pairing. A typical DART also has a C-terminal interchain disulfide bond. The two parental antibodies of a DART have different specificities, typically for a target antigen and CD3, so that the DART is bispecific and serves the same function as a BiTE. BiKEs and TriKEs are analogous to BiTEs but replace the anti-CD3 binding domain with an anti-CD16 binding domain so that instead of engaging T cells they engage NK cells. They may also contain in IL-15 linker between the scFv units instead of or in addition to the anti-CD16 binding domain to provide further NK activation. An ICE is a tetravalent, bispecific engager comprising an anti-CD16A diabody (e.g., a dimer of anti-CD16A scFv) and an anti-tumor antigen diabody (e.g., a dimer of anti-tumor antigen scFv) tandemly connected by peptide linkers (e.g., (Gly-Gly-Ser)₃ linker, SEQ ID NO: 1). The anti-CD16A diabody portion has high binding affinity to CD16A expressed on NK cells and macrophages, while the anti-tumor diabody portion specifically recognizes a surface antigen expressed by a tumor of interest. Thus, the ICE functions to connect innate immune cells (e.g., NK cells, macrophages) and their target tumor cells, thereby activating the killing of the tumor cells through processes such as antibody-dependent cellular cytotoxicity (ADCC) and antibody-dependent cellular phagocytosis (ADCP). See, e.g., Ellwanger et al., MAbs (2019) 11(5):899-918; Reusch et al., MAbs (2014) 6(3):727-738, the entire contents of each of which is incorporated by reference herein. In some embodiments, the ICE is one currently in clinical trial for treatment of various cancers, including, for example, AFM13 (targeting CD30-positive lymphomas) and AFM24 (targeting EGFR-expressing tumors). In certain of these embodiments, the anti-CD16A diabody comprises an scFv that binds efficiently and stably to a unique epitope of the CD16A receptor on NK cells and macrophages without competition from the body's own circulating serum IgG, which has the following amino acids sequences of the VH and VL regions, respectively:

(VH region, SEQ ID NO: 2)
QVQLVQSGAEVKKPGESLKVSCKASGYTFTSYYMHWVRQAPGQGL
EWMGIINPSGGSTSYAQKFQGRVTMTRDTSTSTVYMELSSLRSED
TAVYYCARGSAYYYDFADYWGQGTLVTVSS;
(VL region, SEQ ID NO: 3)
SYVLTQPSSVSVAPGQTATISCGGHNIGSKNVHWYQQRPGQSPVL
VIYQDNKRPSGIPERFSGSNSGNTATLTISGTQAMDEADYYCQVW
DNYSVLFGGGTKLTVL.

As used herein, an immune cell engager encoded in nucleic acid packaged in a tLNP refers to a targeted immune cell engager of TICE.

The engineering agents are amendable to multiplexing in a variety of fashions. At the most basic level, one can package multiple agents in a single species of nanoparticle, or one can package each of multiple agents in its own species of nanoparticle that are then combined in a single formulation. More specifically, multiple nucleic acids may be incorporated into a single nanoparticle by packaging multiple mRNAs (as much as about a dozen), by using bi- or polycistronic mRNA, or by also including a DNA template to be knocked-in to the genome and/or guide RNA, or nucleic acids that serve as BRMs. Alternatively, each nucleic acid can be incorporated into its own species of nanoparticle which can be combined in a single formulation. When the in vivo engineering agent comprises an RNA-guided nuclease or encoded RNA-guided nuclease, a guide RNA, and an encoded CAR or TCR to be knocked-in there is a clear advantage to having all of the components packaged in the same nanoparticle, as that will ensure that they are all present in the same cell to interact with each other. When the in vivo engineering agent comprises multiple immune receptors, they can be similarly effective expressed in the same or separate cells. When both the in vivo engineering agent and the conditioning agent are to be delivered by targeted nanoparticle, they will generally need to be packaged in separate nanoparticles if they are being targeted to different cells (for example, to immune cells and tumor cells, respectively), but in some embodiments could be combined in a single formulation. When both the in vivo engineering agent and the conditioning agent are to be delivered by targeted nanoparticle, they can be packaged in the same nanoparticles if they are being targeted to the same cells (that is, to immune cells).

Generally, the immune cell that is to be engineered is a lymphocyte, such as a T cell (in some instances including or being an NKT cell) or an NK cell.

Some embodiments include temporal limitations describing when the conditioning agent is administered relative to the in vivo engineering agent. When the conditioning agent is said to be administered before or prior to the in vivo engineering agent (a “preconditioning” or often, an activating conditioning agent) it can mean before any dose of the in vivo engineering agent is administered in embodiments in which only a single cycle of treatment is contemplated, either because only one or a few administrations of the in vivo engineering agent is required or because the in vivo engineering agent will be administered repeatedly until some clinical milestone occurs (for example, for cancer, progression, complete response, etc.) and then terminated. However, it can also mean before a first dose of a cycle of the in vivo engineering agent in embodiments entailing cycles of treatment in which the in vivo engineering agent is administered one to several times and then administration is suspended for an interval of time and then reinitiated. The suspension may allow for evaluation or diagnostic procedure, to allow the patient to recover from any adverse effects of the treatment, or to provide an opportunity to repeat the conditioning regimen. If the primary target of the in vivo engineering agent is rapidly expanding cells, such as T cells modified to transiently express a CAR or TCR, in some embodiments, the in vivo engineering agent can be administered every three to four days. If the primary target of the in vivo engineering agent is less rapidly expanding cells, such as NK cells modified to transiently express a CAR or TCR, in some embodiments the in vivo engineering agent can be administered every 7 to 14 days. If the in vivo engineering agent modifies cells to permanently express a reprogramming agent (such as a CAR, TCR, or immune cell engager), then repeated administrations of the in vivo engineering agent may only be needed to increase the number of modified cells, if at all. How long before administration of the in vivo engineering agent the conditioning agent can or should be administered will vary to a degree with the conditioning agent depending on its biologic half-life in the body and how quickly its effects are achieved and persist. However, the effects of the activating conditioning agent, if not the agent itself, should persist during the time interval over which at least some of the administrations of the in vivo engineering agent occur. Thus, in many embodiments, the sole or last dose of an activating conditioning agent is administered about three to about seven days prior to an initial administration of the in vivo engineering agent. In other embodiments, administration of the activating conditioning agent is also administered as a concurrent conditioning agent.

When the conditioning agent is said to be administered concurrently with the in vivo engineering agent, in some embodiments, it means that the conditioning agent is administered on the same day or within the same 24-hour period as the in vivo engineering agent. In other embodiments, when the subject is receiving repeated regular doses of the in vivo engineering agent to provide a continuous presence of engineered cells, it means the conditioning agent is administered at any point in the time interval over which the in vivo engineering agent is administered. If administration of the in vivo engineering agent is suspended and later resumed, administration of the conditioning agent during the period of suspension is not considered concurrent. If administration of the in vivo engineering agent is repeated at time points that are so far apart that there is not a continuous presence of engineered cells, administration of the in vivo engineering agent when engineered cells are not present is not considered concurrent. If the conditioning agent is administered when engineered cells are present, then it is considered concurrent. Although Treg cell depleting agents and the γ-chain receptor cytokines are presented as pre-treatment conditioning agents, in some embodiments, their use is continued concurrently with the in vivo engineering agent.

When the conditioning agent is said to be administered after the in vivo engineering agent, in some embodiments, it means that the conditioning agent is administered at least one day after a single administration of the in vivo engineering agent. In other embodiments, when the subject is receiving repeated regular doses of the in vivo engineering agent to provide a continuous presence of engineered cells, it means the conditioning agent is administered after such regular administration is suspended or terminated.

As used herein “mobilization” refers to the movement of immune cells from secondary lymphoid organs into the bloodstream and also from the bloodstream into a tumor or other locus of disease. The herein disclosed conditioning regimens promoting mobilization are primarily concerned with the latter, but the former effect is not excluded and indeed may contribute to the latter effect. The function of mobilizing immune cells has several facets into which it can be subdivided.

In one facet mobilization can bring reprogrammed lymphocytes into a tumor or other locus of disease, where the lymphocyte encountered the in vivo engineering agent elsewhere in the body. This can be accomplished with γ-chain receptor cytokines, inflammatory chemokines, and systemically administered immune checkpoint inhibitors which thus constitute means for mobilizing reprogrammed lymphocytes.

In another facet, mobilization can recruit lymphocytes into a tumor or other locus of disease to be engineered there. This can be accomplished with γ-chain receptor cytokines, inflammatory chemokines, and inhibitors of CTLA-4 which thus constitute means for mobilizing lymphocytes to a locus of disease (including a tumor).

In another facet, mobilization can bring endogenous T lymphocytes with specificity for a relevant antigen (such as a tumor antigen) into a tumor or other locus of disease. Multi-antigen attack will generally be more effective than single antigen attack so that such endogenous immunity will augment the effectiveness of the reprogrammed immune cells. This can be accomplished with γ-chain receptor cytokines, inflammatory chemokines, and targeted and systemically administered immune checkpoint inhibitors which thus constitute means for mobilizing endogenous T cell immunity.

In yet another facet, mobilization can bring NK cells into the locus of disease to act in concert with the reprogrammed T cells. (Depending on the targeting moiety on the in vivo reprogramming agent the NK cells may or may not include reprogrammed NK cells). This can be accomplished with inflammatory chemokines and IL-15 which thus constitute means for mobilizing NK cells.

In still another facet, mobilization can bring monocytes/macrophages into the locus of disease to destroy diseased tissue. This can be accomplished with systemic and targeted immune checkpoint inhibitors and inflammatory chemokines which thus constitute means for mobilizing monocytes/macrophages.

In again another facet, mobilization can recruit antigen presenting cells to the tumor or other locus of disease to promote epitope spreading. This can be accomplished with systemic and targeted immune checkpoint inhibitors and inflammatory chemokines which thus constitute means for mobilizing antigen presenting cells.

Collectively, the BRMs accomplishing each of these facets of mobilization constitute means for mobilizing immune cells.

Certain aspects include a pre-treatment conditioning regimen for priming the immune system prior to in vivo reprogramming comprising systemic administration of a γ-chain receptor agonist. In some embodiments, the agonist is a γ-chain receptor cytokine. In some embodiments, the agonist is peptide ligand of the receptor. In some embodiments, the systemic administration is by intravenous or subcutaneous infusion or injection. The γ-chain receptor cytokine is delivered in one or multiple doses prior to administration of an in vivo engineering agent. In some embodiments, three weekly administrations of the γ-chain receptor cytokine are made with the final administration three to seven days before scheduled (or actual) administration of the in vivo engineering agent. In various embodiments, the γ-chain receptor cytokine comprises IL-15, IL-2, IL-7, or IL-21. The γ-chain receptor cytokines have multiple effects on lymphocytes, such as T cells and NK cells, including expansion, activation to polyfunctionality, and mobilization from secondary lymphoid organs to the bloodstream and into sites of pathologic effect, including tumors. Accordingly, γ-chain receptor cytokines constitute means for expanding, activating to polyfunctionality, and/or mobilizing effector cells, for example, T and/or NK cells. Similarly, they constitute means for γ-chain receptor mediated signaling, means for expanding polyfunctional effector cells, and means for mobilizing immune effector cells in one or more of the disclosed mobilization facets, as appropriate. Some embodiments specifically include one or more of the γ-chain receptor cytokines (IL-2, IL-4, IL-7, IL-9, IL-15, and interleukin-21). Some embodiments specifically exclude include one or more of the γ-chain receptor cytokines (IL-2, IL-4, IL-7, IL-9, IL-15, and interleukin-21). UniProt accessions P60568, P05112, P13232, P15248, P40933, and Q9HBE4, each of which is incorporated by reference in its entirety, provide examples of amino acid sequences for IL-2, IL-4, IL-7, IL-9, IL-15, and IL-21, respectively. In some embodiments, a peptide mimetic can be used instead of the cytokine and in further embodiments the peptide with receptor binding activity can be incorporated into a fusion protein, for example by fusing it to the Fc portion of an antibody. Peptides with affinity for IL-7R, IL-2Rα, IL-2Rβ, and IL-2Rγc (the γ-chain receptor) are disclosed in US Patent Application Publications 20220119493A1, 20220119453A1, and 20220275026A1, each of which is incorporated by reference for all that it teaches about the structure and activity of peptide mimetics that are ligands of cytokine receptors. In some embodiments, the peptide mimetic comprises an IL-7R ligand having the sequence QCIHWDIETLLSCV (SEQ ID NO: 4), VYCAEIGEYRVCRQ (SEQ ID NO: 5), YMACSSGLSLCRLS (SEQ ID NO: 6), GGVPWCTLDPGSLQCAWF (SEQ ID NO: 7), QCVHWDLDTLFGCIREQLEL (SEQ ID NO: 8), VHRIPWCTLDPGGLQCAWLRQM (SEQ ID NO: 9), GGRSCLWQPGALHCTWWAEEEPV (SEQ ID NO: 10), or GWGIPWCTLDPGSLQCAWLGKH (SEQ ID NO: 11). These cytokines and peptide ligand constitute means for activating their particular receptors and means for activating γ-chain receptors generally.

In some embodiments for priming the immune system prior to in vivo reprogramming comprising systemic administration of a γ-chain receptor cytokine, the subject is administered at least an initial dose of the in vivo engineering agent. In some embodiments, the subject is one who receives at least one dose of an in vivo engineering agent. In some embodiments, the subject is one who receives at least one dose of an in vivo engineering agent after having received a last dose of the γ-chain cytokine three to seven days previously. Such schedules may be repeated in multiple cycles of treatment. In some embodiments, the γ-chain receptor cytokine is administered prior to an initial administration of the in vivo engineering agent. In some embodiments, the γ-chain receptor cytokine is administered prior to each individual administration of the in vivo engineering agent or prior to each individual group of administrations (for example, two to five administrations every three to four days) of the in vivo engineering agent. In some embodiments, the γ-chain receptor cytokine is administered prior to any administration of the in vivo engineering agent occurring more than two, three, or four weeks, or one, two, three, or four months after the most recent administration of the γ-chain receptor cytokine.

An in vivo engineering agent administered subsequently to the systemically administered γ-chain cytokine reprograms a greater number of cells, the reprogrammed cells are more effectively deployed due to the increased mobilization, and the proportion of reprogrammed cells that are polyfunctional is increased, as compared to the in vivo engineering agent administered without the prior conditioning. CAR-T therapy has so far been utilized primarily with hematologic cancers such as diffuse large B cell lymphoma. However, with this activating conditioning regimen, increased numbers and percentage of effector cells are observed not only systemically, but in the target tissue such as a solid tumor, as well.

Pre-treatment conditioning with systemically administered γ-chain cytokines can also be used in combination with a variety of other cancer therapies including other immunotherapies (such as immune checkpoint inhibition therapy), targeted therapies (such as with kinase inhibitors), chemotherapies, radiotherapies, or cell-based therapies (such as adoptive transfer of CAR- or TCR-modified immune cells, tumor infiltrating lymphocytes (TIL), monocytes, or macrophages). This pre-treatment regimen can also be used in combination with treatments for autoimmune or fibrotic disorders.

Certain aspects include a pre-treatment conditioning regimen for priming the immune system prior to in vivo reprogramming comprising targeted or tropic administration of a nucleic acid encoding a γ-chain receptor cytokine. The γ-chain receptor cytokine-encoding nucleic acid is delivered in one or multiple doses prior to, administration of an in vivo engineering agent or other therapy. In some embodiments, the γ-chain receptor cytokine-encoding nucleic acid may additionally be administered concurrently with the in vivo engineering agent. In some embodiments, three weekly administrations of the γ-chain receptor cytokine-encoding nucleic acid are made with the final administration three to seven days before scheduled (or actual) administration of the in vivo engineering agent. In various embodiments, the γ-chain receptor cytokine comprises IL-15, IL-2, IL-7, or IL-21. In some embodiments, the γ-chain receptor cytokine is provided as encoding mRNA packaged in a targeted or tropic nanoparticle. In other embodiments, the γ-chain receptor cytokine is provided encoded in a (non-mRNA) nucleic acid vector packaged in a targeted or tropic nanoparticle. In some embodiments, the nanoparticle is a lipid nanoparticle. In some embodiments, the targeted nanoparticle in which the γ-chain receptor cytokine is provided comprises a binding moiety for a tumor antigen expressed by the tumor to be treated. As noted above, the γ-chain receptor cytokines have multiple effects on lymphocytes, such as T cells and NK cells, including expansion, activation to polyfunctionality, and mobilization from secondary lymphoid organs to the bloodstream and into sites of pathologic effect, including tumors. Accordingly, targeted and tropic nanoparticles comprising encoded γ-chain receptor cytokines constitute nanoparticle means for expanding, activating to polyfunctionality, and/or mobilizing T and/or NK cells. Similarly, they constitute means for γ-chain receptor mediated signaling, means for expanding polyfunctional effector cells, and means for mobilizing immune effector cells in one or more of the disclosed mobilization facets, as appropriate. The nucleic acids themselves can be termed encoded means for the same functions. Some embodiments specifically include one or more of the encoded γ-chain receptor cytokines (IL-2, IL-4, IL-7, IL-9, IL-15, and interleukin-21). Some embodiments specifically exclude one or more of the encoded γ-chain receptor cytokines (IL-2, IL-4, IL-7, IL-9, IL-15, and interleukin-21). Some embodiments specifically include or exclude one or more species of tropic or targeted nanoparticle. In some embodiments, the subject is administered at least an initial dose of an in vivo engineering agent. In some embodiments, the subject is one who receives at least one dose of an in vivo engineering agent. In some embodiments, the subject is one who receives at least one dose of an in vivo engineering agent after having received a last dose of the γ-chain cytokine-encoding nucleic acid three to seven days previously. Such schedules may be repeated in multiple cycles of treatment. In some embodiments, the encoded γ-chain receptor cytokine is administered prior to an initial administration of the in vivo engineering agent. In some embodiments, the encoded γ-chain receptor cytokine is administered prior to each individual administration of the in vivo engineering agent or prior to each individual group of administrations (for example, two to five administrations every three to four days) of the in vivo engineering agent. In some embodiments, the encoded γ-chain receptor cytokine is administered prior to any administration of the in vivo engineering agent occurring more than two, three, or four weeks, or one, two, three, or four months after the most recent administration of the encoded γ-chain receptor cytokine.

An in vivo engineering agent administered subsequently to the tropic or targeted administration of γ-chain cytokine reprograms a greater number of cells, the reprogrammed cells are more effectively deployed due to the increased mobilization, and the proportion of reprogrammed cells that are polyfunctional is increased, as compared to the in vivo engineering agent administered without the prior conditioning. CAR-T therapy has so far been utilized primarily with hematologic cancers such as diffuse large B cell lymphoma. However, with this activating conditioning regimen, increased numbers and percentage of effector cells are observed primarily in the target tissue such as a solid tumor, though they may be observed systemically as well.

Pre-treatment conditioning with tropic or targeted administration of γ-chain cytokines can be used in combination with a variety of cancer therapies including other immunotherapies (such as immune checkpoint inhibition therapy), targeted therapies (such a with kinase inhibitors), chemotherapies, radiotherapies, or cell-based therapies (such as adoptive transfer of CAR- or TCR-modified immune cells, tumor infiltrating lymphocytes (TIL), monocytes, or macrophages). This pre-treatment regimen can also be used in combination with treatments for autoimmune or fibrotic disorders.

Certain aspects include a method of conditioning or priming a subject to receive an in vivo engineering agent to reprogram the immune system by expanding, activating to polyfunctionality, and/or redistributing T cells or NK cell by providing a γ-chain receptor cytokine prior to administration of the in vivo engineering agent. In some embodiments, the γ-chain receptor cytokine is provided by systemic administration of the cytokine. In some embodiments, the γ-chain receptor cytokine is provided by administration of a tropic or targeted nanoparticle comprising a nucleic acid encoding the γ-chain receptor cytokine. In some embodiments, the nucleic acid is an mRNA. Other features of these aspects correspond to those described for the γ-chain receptor cytokine aspects above.

One aim in the engineering of immune cells is that a substantial proportion of the cells transformed be polyfunctional effector cells. Polyfunctional effector cells are those that at the single cell level have the ability to secrete multiple cytokines and chemokines and mediate cytolysis (for example, by the secretion of granzymes). Initially these functions were viewed to be exhibited simultaneously, and the functions reflected integrative measurement from assessment at the end of an experiment. More recent studies based on measurements made throughout an experiment reveal that typically the different activities are expressed one at a time in a programmed manner related to the differentiation state of the cells (for example, naïve CD4⁺ T cells versus effector memory cells, etc.) Polyfunctionality can be assessed with proteomics assay systems (such as ISOPLEXIS ISOSPARK), multicolor intracellular cytokine staining in flow cytometry, or single-cell RNA sequencing. Polyfunctionality has been shown to correlate with T cell efficacy and immune protection. Increased potency of the reprogrammed cells can be assessed with the above methods in conjunction with co-culture bioassays as well as immunohistochemical analysis and the like to assess the cells in situ.

The benefits of conditioning related to polyfunctionality can arise from two effects. Simply increasing the number of polyfunctional cells available to be engineered will lead to an increase in the total number of engineered cells even if the intrinsic per cell efficiency of engineering remains unchanged. However, the metabolic activation that occurs when expanding cells and inducing polyfunctionality can also elevate the nanoparticle handling capacity of the cell making them more susceptible to engineering; that is, the intrinsic per cell efficiency of engineering is increased.

Certain aspects include a conditioning regimen to facilitate in vivo reprograming of the immune system comprising systemic administration of an immune checkpoint inhibitor. In some embodiments, the immune checkpoint inhibitor comprises an anti-CTLA-4 antibody. In some embodiments, the immune checkpoint inhibitor comprises an anti-PD-1, anti-PD-L1, and Tim-3 or anti-LAG-3 antibody. In some embodiments, immune checkpoint inhibitors can be referred to as means for releasing an immune checkpoint or means for inhibiting an immune checkpoint. Immune checkpoint inhibition can bring about multiple effects including reduction of the number or functionality of Treg cells mediating immune suppressive effects, activation or functional enablement of immune cells (such as T effector cells), promotion of broader immune responses including epitope spreading, and facilitation of redistribution of immune cells to organs or tissues of interest such as the tumor microenvironment. Accordingly, immune checkpoint inhibitors constitute means for broadening an immune response, means for mobilizing immune effector cells in one or more of the disclosed mobilization facets, as appropriate, and means for reducing immune suppression. Some embodiments specifically include or exclude one or more immune checkpoint inhibitors (inhibitors or the checkpoint associated with CTLA-4, PD-1, PD-L1, Tim-3 LAG-3, OX40, GITR, CD40, CD122, CD137, CD122, CD40, ICOS, TIGIT, Siglec-15, or B7H3).

Commonly, immune checkpoint inhibitors are administered by intravenous or subcutaneous infusion of the antibody (or other molecule), however, use of encoding nucleic acid vectors or mRNA are also possible. In some embodiments, the method comprises administration of at least one dose of an in vivo engineering agent. In some embodiments, the subject is one who receives or has received at least one dose of an in vivo engineering agent. Immune checkpoint inhibitor antibodies are often administered from one to two times per month (for example, every three weeks). In some embodiments, the immune checkpoint inhibitor is administered twice, three weeks apart. In various embodiments, such immune checkpoint inhibitor administration schedules can be implemented to precede, overlap, or follow administration of the in vivo engineering agent. In some embodiments, a second or greater administration of the immune checkpoint inhibitor takes place one week prior to a scheduled (or actual) initial administration of the in vivo engineering agent. In some embodiments, the final administration of the immune checkpoint inhibitor takes place the same day (plus or minus one day) as the initial administration of the in vivo engineering agent. In some embodiments, an initial administration of the immune checkpoint inhibitor takes place two days to two weeks after the initial administration of the in vivo engineering agent. In some embodiments, the final administration of the immune checkpoint inhibitor takes place two days to two weeks after the initial administration of the in vivo engineering agent. In some embodiments, a second dose of the immune checkpoint inhibitor is administered one week prior to the initial administration of the in vivo engineering agent and a third dose is administered two weeks after the initial administration of the in vivo engineering agent. Such schedules may be repeated in multiple cycles of treatment.

An in vivo engineering agent administered in conjunction with immune checkpoint inhibitor conditioning will benefit from activation or functional enablement of the reprogrammed cells, reduction in the number of Treg cells opposing the activity of the reprogrammed cells, recruitment of broader immunity including epitope spreading, and mobilization of immune cells into tissues or organs of interest (where the cells targeted by the in vivo engineering agent reside). Accordingly, the reprogrammed cells have a greater proportion of polyfunctional cells and are deployed to the targeted tissue or organ in greater numbers as compared to subjects not receiving a conditioning regimen. Additionally, the combination of the immune attack from the reprogrammed cells and the immune checkpoint inhibition leads to a more profound (that is, accomplishing greater removal of pathogenic cells, for example, greater tumor regression or reduced autoimmunity) and durable response to the pathogenic cells through both non-antigen-specific and antigen-specific effectors.

In addition to oncologic treatment, this immune checkpoint inhibitor conditioning regimen can also be combined with immune reprogramming treatments for autoimmune and fibrotic disorders.

Certain aspects include a conditioning regimen to facilitate in vivo reprograming of the immune system, or to augment other therapies, comprising targeted or tropic administration of a nucleic acid-encoded immune checkpoint inhibitor. In some embodiments, the immune checkpoint inhibitor comprises an anti-CTLA-4 antibody. In some embodiments, the immune checkpoint inhibitor comprises an anti-PD-1, anti-PD-L1, and Tim-3 or anti-LAG-3 antibody. In some embodiments, the immune checkpoint inhibitor is provided as encoding mRNA packaged in a targeted or tropic nanoparticle. In other embodiments, the immune checkpoint inhibitor is provided encoded in a (non-mRNA) nucleic acid vector packaged in a targeted or tropic nanoparticle. In some embodiments, the nucleic acid encoded immune checkpoint inhibitors can be referred to as encoded means for releasing an immune checkpoint or encoded means for inhibiting an immune checkpoint. Immune checkpoint inhibition can bring about multiple effects including reduction of the number or functionality of Treg cells mediating immune suppressive effects, activation or functional enablement of immune cells (such as T effector cells), promotion of broader immune responses including epitope spreading, and facilitation of redistribution of immune cells to organs or tissues of interest such as the tumor microenvironment. Accordingly, targeted and tropic nanoparticles comprising encoded immune checkpoint inhibitors constitute nanoparticle means for broadening an immune response, means for mobilizing immune effector cells in one or more of the disclosed mobilization facets, as appropriate, and means for reducing immune suppression. Some embodiments specifically include or exclude one or more immune checkpoint inhibitors (inhibitors or the checkpoint associated with CTLA-4, PD-1, PD-L1, Tim-3 LAG-3, OX40, GITR, CD40, CD122, CD137, CD122, CD40, ICOS, TIGIT, Siglec-15, or B7H3). The targeted nanoparticle in which the encoded immune checkpoint inhibitor is provided comprises a binding moiety for a tumor antigen expressed by the tumor, or for a marker expressed by another pathogenic tissue, to be treated.

The nanoparticle in which the encoded immune checkpoint inhibitor is provided can be administered by intravenous, intraperitoneal, or intralesional infusion or injection. In some embodiments, the nanoparticle in which the encoded immune checkpoint inhibitor is provided is administered on a schedule similar to the systemically administered immune checkpoint inhibitor. In other embodiments, the nanoparticle in which the encoded immune checkpoint inhibitor is provided is administered every 3, 4, 5, or 6 days or weekly for a period of as much as one month, for example, for 1, 2, 3, or 4 weeks, to ensure that an adequate concentration of immune checkpoint inhibitors is achieved and maintained within the tumor or other locus of disease. In some embodiments, a first administration of the immune cell in vivo engineering agent occurs about two weeks after the first administration of the nanoparticle comprising the nucleic acid encoding the immune checkpoint inhibitor.

This conditioning regimen comprising administration of a nanoparticle in which the encoded immune checkpoint inhibitor can be used in combination with a variety of cancer therapies including immunotherapies (such as CAR-, TCR-, and immune checkpoint inhibition therapy), targeted therapies (such a with kinase inhibitors), chemotherapies, radiotherapies, or cell-based therapies (such as adoptive transfer of CAR- or TCR-modified immune cells, tumor infiltrating lymphocytes (TIL), monocytes, or macrophages) and can be practiced before the treatment, concurrently with the treatment, following the treatment, or some combination thereof. In some embodiments, the method comprises administration of at least one dose of an in vivo engineering agent or other treatment. In some embodiments, the subject is one who receives or has received at least one dose of an in vivo engineering agent or other treatment. In general, the targeted or tropic encoded immune checkpoint inhibitor can be administered on the same schedules, and in combination with the same treatments, as described for the immune checkpoint inhibitor antibodies above. Moreover, similar if more localized effects can be elicited but with reduced potential for adverse effects than with systemic administration. In particular, local exposure to an immune checkpoint inhibitor within the tumor microenvironment (or other diseased tissue) 1) enables activity of local immune cells; 2) recruits additional immune cells in the microenvironment; and 3) interferes with the activity of Treg cells.

In some embodiments, the BRM is an immune checkpoint inhibitor. In some embodiments, conditioning is combined with immune checkpoint inhibition therapy. Immune checkpoint inhibition therapy refers to the use of pharmaceuticals, typically biologics, that act on regulatory pathways in the differentiation and activation of T cells to promote the passage of T cell development through these checkpoints so that anti-tumor (or other therapeutic) activity can be realized. The agents bringing about immune checkpoint therapy are commonly called immune checkpoint inhibitors and it should be understood that it is the check on T cell development that is being inhibited. Thus, while many immune checkpoint inhibitors also inhibit the interaction of receptor-ligand pairs (e.g., programmed cell death 1 (PD-1) interaction with programmed death ligand 1 (PD-L1)), other checkpoint inhibitors (such as anti-OX40, anti GITR, anti-CD137, anti-CD122, anti-CD40, and anti-ICOS) act as agonists of their targets which release or otherwise inhibit the check on T cell development, ultimately promoting effector function and/or inhibiting regulatory function.

Programed death-1 (PD-1) is a checkpoint protein on T cells. Antibodies against both PD-1 and its binding partner programmed death-ligand 1 (PD-L1) have been used clinically as immune checkpoint inhibitors (PD-1 blockade). Non-limiting examples of monoclonal antibodies (mAbs) that target PD-1/PD-L1 include: the anti-PD-1 mAbs nivolumab (OPDIVO®, Bristol-Myers Squibb), pembrolizumab (KEYTRUDA®, Merck & Co.), cemiplimab-rwlc (LIBTAYO®, Regeneron Pharmaceuticals), and the anti-PD-L1 mAbs durvalumab (MEDI4736, IMFINZI™ Medimmune), atezolizumab (MPDL3280A; TECENTRIQ®, Hoffmann-La Roche), avelumab (BAVENCIO®, EMD Serono), and BMS-936559 (Bristol-Myers Squibb) and others disclosed herein below. These may be referred to as means for PD-1 blockade, means for inhibiting PD-1/PD-L1 binding, or means for immune checkpoint inhibition.

CTLA-4 is an immune checkpoint molecule expressed on the surface of CD4 and CD8 T cells and on CD25+, FOXP3+ T regulatory (Treg) cells. Non-limiting examples of monoclonal antibodies that target CTLA-4 include ipilimumab (YERVOY®; Bristol-Myers Squibb), tremelimumab (Medimmune), bavunalimab, botensilimab, nurulimab, quavonlimab, tuvonralimab, vudalimab, zalifrelimab, JMW-3B3, VH5:VK4, davoceticept, and others disclosed herein below. These may be referred to as means for inhibiting CTLA-4 or means for immune checkpoint inhibition.

TIM-3 (T-cell immunoglobulin and mucin-domain containing-3) is a molecule selectively expressed on IFN-γ-producing CD4⁺ T helper 1 (Th1) and CD8⁺ T cytotoxic 1 (Tc1) T cells. Non-limiting, exemplary antibodies to TIM-3 are disclosed in U.S. Patent Application Publication 20160075783 which is incorporated by reference herein for all it contains regarding anti-TIM-3 antibodies that is not inconsistent with the present disclosure. Other anti-TIM-3 antibodies include TSR-022 (Tesaro) and others disclosed herein below. These may be referred to as means for inhibiting TIM-3 or means for immune checkpoint inhibition.

LAG-3 (lymphocyte-activation gene 3; CD223) negatively regulates cellular proliferation, activation, and homeostasis of T cells, in a similar fashion to CTLA-4 and PD-1 and plays a role in Treg suppressive function. Non-limiting exemplary antibodies to LAG-3 include GSK2831781 (GlaxoSmithKline), relatlimab (BMS-986016, Bristol-Myers Squibb), and others disclosed herein below, as well as the antibodies disclosed in U.S. Patent Application Publication 2011/0150892 which is incorporated by reference herein for all it contains regarding anti-LAG-3 antibodies that is not inconsistent with the present disclosure. These may be referred to as means for inhibiting LAG-3, or means for immune checkpoint inhibition.

TIGIT (T cell immunoreceptor with Ig and ITIM domains) is an immunoreceptor inhibitory checkpoint that has been implicated in tumor immunosurveillance. It competes with immune activating receptor CD226 (DNAM-1) for the same set of ligands: CD155 (PVR or poliovirus receptor) and CD112 (Nectin-2 or PVRL2). Anti-TIGIT antibodies have demonstrated synergy with anti-PD-1/PD-L1 antibodies in pre-clinical models. Tiragolumab (Roche), etigilimab (OncoMed), vibostolimab (MK-7684; Merck), and EOS-448 (iTeos Therapeutics) and others disclosed herein below are non-limiting examples of an anti-TIGIT antibodies. They may be referred to as means for inhibiting TIGIT or means for immune checkpoint inhibition.

GITR (glucocorticoid-induced TNFR-related protein) promotes effector T cell functions and inhibits suppression of immune responses by regulatory T cells. As with OX-40, mentioned above, the checkpoint inhibitor is an agonist of the target, in this case GITR. An agonistic antibody, TRX518 is currently undergoing human clinical trials in cancer. While by itself it may not be sufficient to mediate substantial clinical improvement in advanced cancer, combination with other checkpoint inhibition, such as PD-1 blockade was promising. Further exemplary antibodies to GITR include efaprinermin, efgivanermin, ragifilimab, INCAGN01876 and others disclosed herein below. These antibodies may be referred to as means for inhibiting GITR, or means for immune checkpoint inhibition.

Other immune checkpoint inhibitor targets include, but are not limited to, B- and T-cell attenuator (BTLA), CD40, CD122, inducible T-cell costimulator (ICOS), OX40 (tumor necrosis factor receptor superfamily, member 4), Siglec-15, B7H3, CD137 (4-1 BB; as with CD40 and OX40, checkpoint inhibition is accomplished with an agonist) and others are potentially useful in the disclosed methods. Several anti-OX40 agonistic monoclonal antibodies are in early phase cancer clinical trials including, but not limited to, MEDI0562 and MED16469 (Medimmune), MOXR0916 (Genetech), and PF-04518600 (Pfizer); as is an anti-ICOS agonistic antibody, JTX-2011 (Jounce Therapeutics). Other anti-ICOS (CD278) antibodies include alomfilimab, feladilimab, feladilimab, and the bispecific antibody acazicolcept. Anti-CD40 agonistic antibodies under clinical investigation include dacetuzumab, CP-870,893 (selicrelumab), and Chi Lob 7/4. Anti-siglec-15 antibodies are also known (see, for example, U.S. Pat. No. 8,575,531). Anti-CD137 agonistic antibodies include, but are not limited to, urelumab and utomilumab. Additionally, CD122 has been targeted in cancer clinical trials with bempegaldesleukin (NKTR-214, a pegyltated-IL-2 used as a CD122-biased agonist). B7H3 has been targeted both for immune checkpoint inhibition and as a tumor antigen with reagents such as enoblituzumab, ¹³¹I-omburtamab, ¹⁷⁷Lu-DTPA-omburtamab, ¹³¹I-8H9, ¹²⁴I-8H9, MCG018, and DS-7300a. These may be referred to as means for immune checkpoint inhibition or means for inhibiting (or activating (agonizing), as appropriate) their respective targets. Some embodiments can specifically include or exclude one or more immune checkpoint inhibitor.

Certain aspects include a conditioning regimen to facilitate in vivo reprograming of the immune system, or to augment other therapies, comprising targeted administration of an inflammatory chemokines. Chemokines are generally classified as homeostatic or inflammatory; the latter are generally more appropriate as the conditioning agent in these aspects and include CCL2, CCL3, CCL4, CCL5, CCL11, CXCL1, CXCL2, CXCL-8, CXCL9, CXCL10, and CXCL11. In some embodiments, the chemokine comprises CCL5. In some embodiments, the chemokine comprises CXL9, CXL10 or CXL11. Expression of these inflammatory chemokines results in the local recruitment and expansion of T cells and other immune cells. This expansion can be used to provide cells for reprogramming as well as to augment a variety of other treatments including other immunotherapies (such as immune checkpoint inhibition therapy), targeted therapies (such a with kinase inhibitors), chemotherapies, radiotherapy, or cell-based therapy (such as adoptive transfer of CAR- or TCR-modified immune cells, tumor infiltrating lymphocytes (TIL), monocytes, or macrophages).

In some embodiments, the inflammatory chemokine is provided as encoding mRNA packaged in a targeted or tropic nanoparticle. In other embodiments, the chemokine is provided encoded in a (non-mRNA) nucleic acid vector packaged in a targeted or tropic nanoparticle. In some embodiments, the targeted nanoparticle in which the chemokine is provided comprises a binding moiety for a tumor antigen expressed by the tumor to be treated. In some instances, the tumor antigen is a surface antigen on a neoplastic tumor cell while in other instances the tumor antigen is surface antigen on a stromal tumor cell. In some embodiments the chemokines can be referred to as means for recruiting (or for attracting) and locally expanding immune cells, monocytes/macrophages (CCL2, CCL3, CCL5, CCL7, CCL8, CCL13, CCL17 and CCL22), mast cells (CCL2 and CCL5), neutrophils (CXCL8), eosinophils (CCL11, CCL24, CCL26, CCL5, CCL7, CCL13, and CCL3), or T cells (CCL2, CCL1, CCL22, CCL17, CXCL9, CXCL10 and CXCL11). UniProt accessions P22362, P13500, P10147, P13501, P80098, P80075, P51671, Q99616, 092583, 0006226, 000175, and Q9Y258, each of which is incorporated by reference in its entirety, provide examples of amino acid sequences for CCL1, CCL2, CCL3, CCL5, CCL7, CCL8, CCL11, CCL13, CCL17, CCL22, CCL24, and CCL26, respectively. UniProt accessions P10145, Q07325, P02778, and 014625, each of which is incorporated by reference in its entirety, provide examples of amino acid sequences for CXCL8, CXCL9, CXCL10, and CXCL11, respectively. Some embodiments specifically include or exclude one or more species of chemokine.

The targeted nanoparticle providing the inflammatory chemokine can be administered by intravenous, intraperitoneal, or intralesional infusion or injection. Generally, targeted nanoparticle providing the inflammatory chemokine will be administered several times (for example two, three, or four times) at three- to four-day intervals prior to a first administration of the in vivo engineering agent which will be administered following the last provision of the inflammatory chemokine, for example, on the following day. In some embodiments, the method comprises administration of at least one dose of an in vivo engineering agent. In some embodiments, the subject is one who receives at least one dose of an in vivo engineering agent. In some embodiments, the subject is one who receives at least one dose of an in vivo engineering agent after having received a most recent administration of an mRNA encoding an inflammatory chemokine packaged in a targeted nanoparticle one day before. For extended treatment with the in vivo engineering agent the above schedules can be repeated in multiple cycles, one cycle after another or with pauses for patient rest and evaluation between cycles when the in vivo engineering agent is administered repeatedly, for example in a cluster of doses. Generally, such multiple doses of the in vivo engineering agent are scheduled so they all occur within four to ten days of the most recent provision of the inflammatory chemokine. Alternatively, extended treatment with the in vivo engineering agent can be accomplished by interposing an administration of the targeted nanoparticle providing the chemokine between every 1, 2, or 3 administrations of the in vivo engineering agent, where those administrations occur, for example, every 3 to 4 days. These pre-treatment regimens can be used in combination with treatments for hematologic cancers, solid tumors, autoimmune diseases, and fibrotic disorders.

Certain aspects include a conditioning regimen to facilitate in vivo reprograming of the immune system, or to augment other therapies, comprising systemic or targeted administration of an agent that enhances the activity of antigen presenting cells. In some embodiments, the agent that enhances the activity of antigen presenting cells comprises Flt3 ligand. In some embodiments, the agent that enhances the activity of antigen presenting cells comprises gm-CSF, or IL-18. UniProt accessions P49771, P04141, and Q14116, each of which is incorporated by reference in its entirety, provide examples of amino acid sequences for Flt3 ligand, gm-CSF, and IL-18, respectively.

Flt3 ligand, gm-CSF, and IL-18 constitute means for enhancing the activity of antigen presenting cells or means for recruiting or activating antigen presenting cells. Some embodiments specifically include or exclude one of these classes or species of agent. In some embodiments, the agent that enhances the activity of antigen presenting cells, or mRNA encoding the agent, is packaged in a nanoparticle targeted to or that has tropism for a tumor cell. In some instances, the tumor antigen is a surface antigen on a neoplastic tumor cell while in other instances the tumor antigen is a surface antigen on a stromal tumor cell. For nucleic acid encoded agents an miRNA target domain can be included to restrict or modulate translation of the agent that enhances the activity of antigen presenting cells in non-targeted cells or tissues. For example, miRNA 122 will suppress translation of an mRNA containing its target domain in liver cells (hepatocytes) as will the miRNAs 96, 185, and 223, and miRNA 142 can be exploited in like fashion in Kupffer cells. Expression can be similarly suppressed in myeloid and reticuloendothelial system cells by including a target domain for miRNAs 100, 125a, 125b, 146a, 146b, and 155. The distribution of miRNAs in human tissues is presented in Ludwig et al. Nucleic Acids Research, 44(8): 3865-3877 (2016), and downloadable from the Human miRNA tissue atlas at ccb-webDOTcsDOTuni-saarlandDOTde/tissueatlas/each of which is incorporated by reference in its entirety to the extent that is not inconsistent with the present disclosure.

The agent that enhances the activity of antigen presenting cells can be administered prior to, concurrently with, or subsequent to administration of an in vivo engineering agent. Thus, in some embodiments, the subject has received an in vivo engineering agent prior to administration of the antigen presentation enhancing agent. In some embodiments, the subject is receiving an in vivo engineering agent concurrently with administration of the antigen presentation enhancing agent (concurrently with can indicate on the same day as a single administration of the in vivo engineering agent or within the interval of time in which multiple administrations of the in vivo engineering agent are received). In some embodiments, the subject is one who receives in vivo engineering agent after the antigen presentation enhancing agent has been administered. In some embodiments, the nanoparticles in which the antigen presentation enhancing agent or encoding mRNA is packaged is administered intravenously, while in other embodiments the administration is intraperitoneal or intralesional.

In some embodiments, the nanoparticle comprising a nucleic acid encoding the agent that enhances the activity of antigen presenting cells is administered three to four days and 12 to 24 hours prior to the in vivo immune cell engineering agent. When administered concurrently with the in vivo immune cell engineering agent, in some embodiments, the nanoparticle comprising a nucleic acid encoding the agent that enhances the activity of antigen presenting cells is administered anytime the same day or 12 to 24 hours in advance for each of multiple administrations of the in vivo immune cell engineering agent. When administered subsequent to the in vivo immune cell engineering agent, in some embodiments, the nanoparticle comprising a nucleic acid encoding the agent that enhances the activity of antigen presenting cells it is administered every three to seven days while the tumor is shrinking, thereby promoting epitope spreading. In some instances, the nanoparticle comprising a nucleic acid encoding the agent that enhances the activity of antigen presenting cells is being administered subsequent to a pause in or conclusion of treatment with the in vivo immune cell engineering agent. Systemic administration of the agent that enhances the activity of antigen presenting cells can follow the same schedules.

Activated antigen presenting cells will have heightened capability to activate and expand T cells including polyfunctional effector cells specific for a broad range of antigens. Thus, agents that enhance the activity of antigen presenting cells will not only promote induction of immunity to further tumor (or other disease-associated) antigens (epitope spreading), but as these agents will also increase the number of cells amenable to reprogramming, the number and percentage of such immune effector cells will be increased.

In addition to conditioning subjects who receive, are receiving, or have received an in vivo engineering agent, these conditioning regimens to enhance antigen presentation are also useful in combination with a variety of other cancer therapies including other immunotherapies (such as immune checkpoint inhibition therapy or anti-tumor antigen monoclonal antibody therapy), targeted therapies (such as with kinase inhibitors), and radiotherapies. As any of these therapies can lead to the release of tumor antigens, enhancement of antigen presenting cell activity associated with increased uptake, processing, and presentation of those antigens can result in the broadening the anti-tumor T cell repertoire. Thus, the number and percentage of anti-tumor immune effector cells will be expanded, both locally and systemically. In the context of in vivo reprogramming, when used as pre-conditioning or concurrent conditioning, the number of cells available for reprogramming is increased, while when used concurrently with or after administration of the in vivo reprogramming agent, it can serve as adjuvant conditioning by promoting epitope spreading and recruiting other arms of the immune system.

Certain aspects include a conditioning regimen to facilitate in vivo reprograming of the immune system, or to augment other therapies, comprising targeted administration of a highly active BRM enhancing the activity of all arms of the cellular immune system (for example, a pan-activating cytokine). In some embodiments, the BRM is a cytokine that has dose-limiting toxicity if administered systemically. In some embodiments, the highly active BRM comprises IL-12. In some embodiments, the highly active BRM comprises IL-18. These BRM constitute means for enhancing the activity of all arms of the cellular immune system. In some embodiments, the highly active BRM is provided as encoding mRNA packaged in a targeted or tropic nanoparticle. In other embodiments, the highly active BRM is provided encoded in a (non-mRNA) nucleic acid vector packaged in a targeted or tropic nanoparticle. The targeted nanoparticle in which the highly active BRM is provided comprises a binding moiety for a tumor antigen expressed by the tumor to be treated. In some instances, the tumor antigen is a surface antigen on a neoplastic tumor cell while in other instances the tumor antigen is a surface antigen on a stromal tumor cell. UniProt accessions P29459, P29460, and 014116, each of which is incorporated by reference in its entirety, provide examples of amino acid sequences for IL-12α, IL-12β, and IL-18, respectively.

To further limit systemic toxicity, in some embodiments, the nanoparticle has CD47 or an effective portion to inhibit uptake by untargeted cells anchored on its surface. To avoid uptake by macrophages, and thereby further improve specificity of targeting to the tumor cell, the nanoparticle surface can be decorated with a “don't-eat-me” signal, which inhibits phagocytosis, such as provided by CD47 or active fragments thereof. Accordingly, in some method of treatment embodiments, the targeted nanoparticle is decorated with CD47 or CD47-derived peptides comprising CD47's “don't-eat-me” signal; polypeptides comprising the sequence GNYTCEVTELTREGETIIELK (SEQ ID NO: 12). This will inhibit uptake by Kupffer cells in the liver and by the reticuloendothelial system to optimize the specificity of expression in tumor cells. CD24 or an effective portion thereof also provides a signal to evade phagocytosis. This “don't-eat-me” signal can be delivered by display of the portion of CD24 having the sequence SETTTGTSSNSSQSTSNSGLAPNPTNATTKAA (SEQ ID NO: 13). The external portion of CD47 or CD24, or an effective portion or either CD47 or CD24 to inhibit uptake by untargeted cells can be anchored on the surface by conjugation to a lipid, for example by conjugation to a PEG-lipid as described herein or otherwise known in the art. Their amino acid sequences can be modified with additional non-native amino acids and/or other moieties at the N-terminal end to facilitate attachment to the nanoparticle.

To further limit systemic toxicity, in some embodiments, the mRNA encoding the highly active BRM packaged in the nanoparticle contains an miRNA target domain to inhibit expression in non-target cells. For example, miRNA 122 will suppress translation of an mRNA containing its target domain in liver cells (hepatocytes) as will the miRNAs 96, CD47185, and 223, and miRNA 142 can be exploited in like fashion in Kupffer cells. Expression can be similarly suppressed in myeloid and reticuloendothelial system cells by including a target domain for miRNAs 100, 125a, 125b, 146a, 146b, and 155. The distribution of miRNAs in human tissues is presented in Ludwig et al. Nucleic Acids Research, 44(8): 3865-3877 (2016), and downloadable from the Human miRNA tissue atlas at ccb-webDOTcsDOTuni-saarlandDOTde/tissueatlas/ each of which is incorporated by reference in its entirety to the extent that it is not inconsistent with the present disclosure. The nanoparticle can be administered by intravenous, intraperitoneal, or intralesional infusion or injection. In some embodiments, the highly active BRM is administered prior to the subject receiving an in vivo engineering agent, for example, one or multiple times with the last administration one to seven days beforehand. In some embodiments, the highly active BRM is administered to a subject who has previously received an in vivo engineering agent (for example, within four days). In some embodiments, the method comprises administration of at least one dose of an in vivo engineering agent. In some embodiments, the subject is one who receives or has received at least one dose of an in vivo engineering agent. In some embodiments, the subject is one who receives at least one dose of an in vivo engineering agent after having received a highly active BRM or encoding mRNA packaged in a targeted nanoparticle one to seven days before.

Certain aspects include a conditioning regimen to facilitate in vivo reprograming of the immune system comprising administration of low dose cyclophosphamide. Unlike the other conditioning agents described herein, cyclophosphamide is not a BRM. Rather it is a cytotoxic alkylating agent commonly used as chemotherapeutic in the treatment of several types of cancer. It is also lymphodepletive and used in the treatment of severe autoimmunity and as a conditioning regimen prior to adoptive transfer of T cells such as in bone marrow transplantation and ex vivo generated CAR-T cells. These conditioning protocols use what is considered high-dose cyclophosphamide (260 mg/kg). Low dose metronomic dosing of cyclophosphamide, for example 50 mg daily or 100 mg every other day, does not have the generally lymphodepleting effect of high dose treatment, but does reduce the number or functionality of Treg cells and can also stimulate the activity of antigen presenting cells. Accordingly, the reprogrammed cells can exhibit increased activity and more effective deployment due to the suppression of regulatory T cells. The suppression of Treg cells can also lead to greater effectiveness of endogenous immunity and the increased activity of antigen presenting cells can promote epitope spreading. Altogether this leads to a more profound and durable immune response to the tumor or other targeted cells. This pre-treatment regimen can be used in combination with treatments for hematologic cancers, solid tumors, chronic infections diseases, autoimmune diseases, and fibrotic disorders.

Prior to administration of an in vivo immune engineering agent, a subject who is to receive an in vivo engineering agent is administered metronomic cyclophosphamide, for example 50 mg daily or 100 mg every other day. In some embodiments, the cyclophosphamide is administered over a period of five to eight days, for example, over six days. In other embodiments, the cyclophosphamide is administered at a daily dose of 10-50 mg for up to three days. In some embodiments, the method comprises administration of at least one dose of an in vivo engineering agent. In some embodiments, the subject is one who receives at least one dose of an in vivo engineering agent. In some embodiments, the subject is one who receives at least one dose of an in vivo engineering agent after having received a final dose of the cyclophosphamide three to four days previously.

Unless indicated otherwise above, the various conditioning regimens can be repeated every one to three months as part of repeated cycles of treatment. In some embodiments comprising adjuvant conditioning, the in vivo engineering agent is administered and followed by administration of the adjuvant conditioning agent as a cycle repeated weekly, biweekly, or three to four time a month. Cycles of treatment can be repeated as long as the subject receives a benefit. In some embodiments, disease is eliminated, and treatment is terminated. In some embodiments, disease is not eliminated, but is reduced and held at a stable level (for example, non-progressive cancer) and treatment cycles can be repeated indefinitely. In some embodiments, the treatment ceases to be beneficial and is terminated. In some embodiments, no further improvement in the disease is observed and treatment is suspended but can be resumed if/when disease worsens or recurs.

With respect to the various aspects, in some embodiments, the subject is human.

In many embodiments, targeted nanoparticles are used to deliver the conditioning agent to the tumor or other diseased tissue. Targeted nanoparticles can also be used to deliver the engineering agent to the immune cells to be engineered in vivo. Targeting is accomplished through a specific binding interaction between a binding moiety on the surface of the nanoparticles and a ligand on the surface of the targeted cell. Most often the binding moiety is an antibody or antigen binding portion thereof. Thus, the binding moiety can be a whole antibody, a minibody, an F(ab)2, an F(ab), an scFv, a diabody, a nanobody, and so forth.

A variety of nanoparticles have been used in the art including polymer nanoparticles and lipid nanoparticles (LNPs). Based on lipid composition it has been reported that LNPs can be preferentially directed to specific tissues (although generally less specifically than targeted nanoparticles). Such nanoparticles will be referred to as tropic nanoparticles and represent an alternative to targeted LNPs. The cells or tissue for which the tropic nanoparticle has a tropism will nonetheless be referred to as targeted cells or tissues. Tropic lipid nanoparticles include those comprising SORT lipids as disclosed in U.S. Pat. No. 11,229,609, which is incorporated herein by reference for all that is teaches about lipids conferring tissue tropism and lipid nanoparticles comprising them that is not inconsistent with the present disclosure. US Patent Publication No. 20220218622A1 discloses the adjustment of pKa of ionizable lipids in lipid nanoparticles to effectuate targeted delivery to a specific tissue or organ of the body. US Patent Publication No. 20220218622A1 is incorporated herein by reference for all that it teaches about lipids conferring tissue tropism and lipid nanoparticles comprising them that is not inconsistent with the present disclosure.

With respect to the herein disclosed conditioning regimens utilizing a nanoparticle, the nanoparticle can be a non-viral, synthetic nanoparticle comprising lipids, polymers, and/or lipopolymers. Nucleic acid-based therapeutics (e.g., DNA, siRNA, mRNA, miRNA, ASO, self-replicating RNA) have significant systemic and cellular barriers for efficient delivery into cells. They are highly susceptible to degradation by nucleases in the body and are at risk of rapid clearance by kidneys. Additionally, their negative charge and hydrophilic nature inhibits efficient delivery across the cell membrane. Nanoparticle based delivery systems especially those comprising lipids, polymers and/or lipopolymers help overcome these delivery challenges. The ideal delivery system for nucleic acid should demonstrate efficient encapsulation of the nucleic acid (thus protecting it from nuclease mediated degradation), improving biodistribution and avoiding rapid clearance by kidneys, enabling efficient uptake across the cell membrane and into the cytosol, be biodegradable, and non-immunogenic to be capable of repeat dosing. Non-viral nanoparticle-based systems comprising lipids, polymers or lipopolymers fit these criteria (see for example Yan et al., Journal of Controlled Release 342:241-279 (2022), which is incorporated by reference for all that it teaches about delivery of nucleic acids by non-viral nanoparticles that is not inconsistent with the present disclosure).

Among these, lipid nanoparticle-based systems are most advanced for RNA delivery with 3 currently approved drugs (Onpattro, and two mRNA based COVID vaccines, Comirnaty and Spikevax). Lipid nanoparticles typically consist of an ionizable or a cationic lipid, a phospholipid, cholesterol, and a PEGylated lipid (see for example Hou et al., Nature Reviews Materials 6:1078-1094 (2021), which is incorporated by reference for all that it teaches about delivery of nucleic acids by lipid nanoparticles). Other examples of lipid-based nanoparticles include cationic liposomes or cationic lipoplexes typically comprising a cationic lipid (permanently charged amino lipid) and a co-lipid such as a phospholipid or cholesterol and in some cases a PEGylated lipid or lipid nanoparticles comprising of lipidoids (lipid-like material). Examples of polymer-based nanoparticles for nucleic acid delivery include linear cationic polymers such as polyamino acid-based polymers (e.g., poly-L-Lysine (PLL), polyarginine, polyhistidine), polyethyleneimine (PEI), natural polymers such as chitosan and hyaluronic acid, branched polymer bases systems such as polyamidoamine (PAMAM) dendrimers, and poly-beta amino esters (PBAE). Examples of lipopolymeric or lipid-polymer hybrid nanoparticles include nanoparticles with a polymeric core (e.g., polylactic-co-glycolic acid (PLGA)) and a lipid shell (see for example Byun et al., BioChip J. 2022:1-18 which is incorporated by reference for all that it teaches about delivery of nucleic acids by nanoparticles including polymer and hybrid nanoparticles that is not inconsistent with the present disclosure). Other such hybrid systems include dendrimeric systems like Janus dendrimers that consist of a lipophilic region of linear or branched alkyl chains and polar ionizable amino heads (Zhang et al., J. Am. Chem. Soc. 143: 12315-12327, (2021), which is incorporated by reference for all that it teaches about delivery of nucleic acids by dendrimer nanoparticles that is not inconsistent with the present disclosure).

In some embodiments, the nanoparticle is a lipid nanoparticle (LNP). In some embodiments, the LNP comprises one or more of an ionizable cationic lipid, a phospholipid, a sterol, a co-lipid, and a polyethylene glycol (PEG)-lipid, or combinations thereof, and a functionalized PEG-lipid conjugated to a binding moiety. As used herein, functionalized PEG-lipid refers to a PEG-lipid in which the PEG moiety has been derivatized with a chemically reactive group (such as, maleimide, NHO ester, Cys, azide, alkyne, and the like) that can be used for conjugating a targeting moiety to the PEG-lipid, and thus, to the LNP comprising the PEG-lipid. The functionalized PEG-lipid can be reacted with a binding moiety after the LNP is formed, so that the binding moiety is conjugated to the PEG portion of the lipid. The conjugated binding moiety can thus serve as a targeting domain for the LNP to form a tLNP.

With respect to the LNP or the tLNP, in some embodiments the molar ratio of the lipids is 40 to 60 mol % ionizable cationic lipid: 7 to 30 mol % phospholipid: 20 to 45 mol % sterol: 1 to 30 mol % co-lipid, if present: 0 to 5 mol % PEG-lipid: 0.1 to 5 mol % functionalized PEG-lipid, when present. The functionalized PEG-lipid is conjugated to a binding moiety that specifically binds to CD2, CD5 or CD8, for example.

With respect to the LNP or the tLNP, in various embodiments, the ionizable cationic lipid comprises a lipid with a measured pKa in the LNP of 6 to 7, facilitating ionization in the endosome. In some embodiments the ionizable cationic lipid has a c-pKa from 8 to 11 and c Log D from 9 to 18 or 11-14. In some embodiments, the ionizable cationic lipids have branched structure to give the lipid a conical rather than cylindrical shape. Suitable ionizable cationic lipids are known to those of skill in the art, including those disclosed in US20130022665, US20180170866, US20160095924, US20120264810, U.S. Pat. Nos. 9,061,063, 9,433,681, 9,593,077, 9,642,804, 10,196,637, 10,207,010, 10,383,952, 10,426,737, 11,066,355, 11,246,993, WO2012170952, WO2021026647, WO2017004143, and WO2017075531 each of which is incorporated by reference for all that it teaches about ionizable cationic lipids that is not inconsistent with the present disclosure.

In some embodiments, the ionizable cationic lipid has a structure of Formula 1,

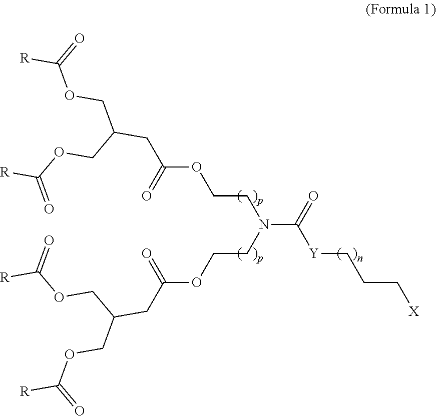

wherein Y is O, NH, N—CH₃, or CH₂,
n is an integer from 0 to 4,
X is

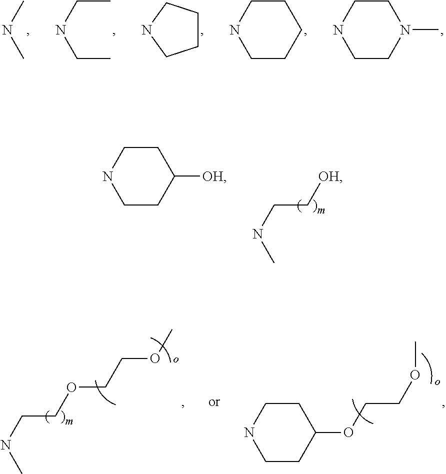

m is an integer from 1 to 3,
is an integer from 1 to 4,
p is an integer from 1 to 4,
wherein when p is 1, each R is independently C₆ to C₁₆ straight-chain alkyl; C₆ to C₁₆ branched alkyl; C₆ to C₁₆ straight-chain alkenyl; C₆ to C₁₆ branched alkenyl; C₉ to C₁₆ cycloalkyl-alkyl in which the cycloalkyl is C₃ to C₈ cycloalkyl positioned at either end or within the alkyl chain; or C₈ to C₁₈ aryl-alkyl in which the aryl is phenyl or naphthalenyl and is positioned at either end or within the alkyl chain,
wherein when p=2, each R is independently C₆ to C₁₄ straight-chain alkyl; C₆ to C₁₄ straight-chain alkenyl; C₆ to C₁₄ branched alkyl; C₆ to C₁₄ branched alkenyl; C₉ to C₁₄ cycloalkyl-alkyl in which the cycloalkyl is C₃ to C₈ cycloalkyl positioned at the either end or within the alkyl chain; or C₈ to C₁₆ aryl-alkyl in which the aryl is phenyl or naphthalenyl and is positioned at either end or within the alkyl chain,
wherein when p=3, each R is independently C₆ to C₁₂ straight-chain alkyl; C₆ to C₁₂ straight-chain alkenyl; C₆ to C₁₂ branched alkyl; C₆ to C₁₂ branched alkenyl; C₉ to C₁₂ cycloalkyl-alkyl in which the cycloalkyl is C₃ to C₈ cycloalkyl positioned at either end or within the alkyl chain; or C₈ to C₁₄ aryl-alkyl in which the aryl is phenyl or naphthalenyl and is positioned at the either end or within the alkyl chain, and
wherein when p=4, each R is independently C₆ to C₁₀ straight-chain alkyl; C₆ to C₁₀ straight-chain alkenyl; C₆ to C₁₀ branched alkyl; C₆ to C₁₀ branched alkenyl; C₉ to C₁₀ cycloalkyl-alkyl in which the cycloalkyl is C₃ to C₈ cycloalkyl positioned at either end or within the alkyl; or C₈ to C₁₂ aryl-alky in which the aryl is phenyl or naphthalenyl and is positioned at the either end or within the alkyl chain.

In some embodiments, the ionizable cationic lipid has the structure below

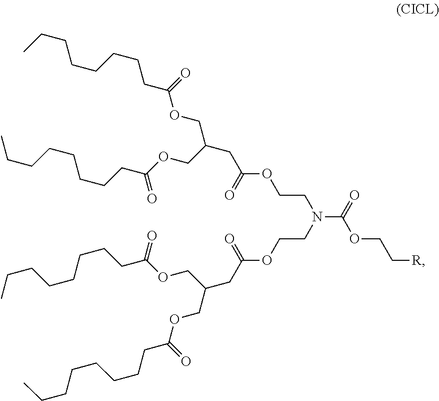

wherein R is

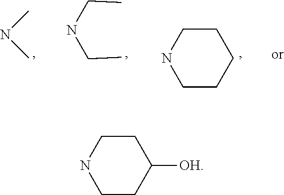

In certain embodiments, the ionizable cationic lipid of CICL is referred to as CICL1 when R is

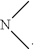

In certain embodiments, the ionizable cationic lipid of CICL is referred to as CICL2 when R is

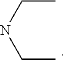

In certain embodiments, the ionizable cationic lipid of CICL is referred to as CICL3 when R is

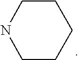

In certain embodiments, the ionizable cationic lipid of CICL is referred to as CICL4 when R is

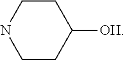

In some embodiments, the ionizable cationic lipid has a structure of Formula 2,

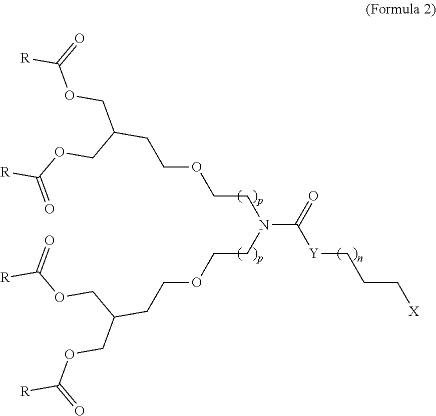

wherein Y is O, NH, N—CH₃, or CH₂,
n is an integer from 0 to 4.
X is

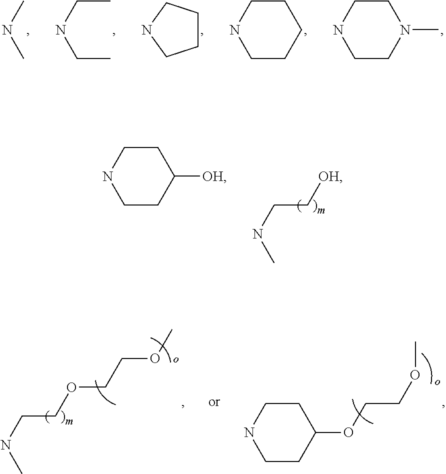

m is an integer from 1 to 3,
is an integer from 1 to 4,
p is an integer from 1 to 4,
wherein when p is 1, each R is independently C₆ to C₁₆ straight-chain alkyl; C₆ to C₁₆ straight-chain alkenyl; C₆ to C₁₆ branched alkyl; C₆ to C₁₆ branched alkenyl; C₉ to C₁₆ cycloalkyl-alkyl in which the cycloalkyl is C₃ to C₈ cycloalkyl positioned at either end or within the alkyl chain; or C₈ to C₁₈ aryl-alkyl in which the aryl is phenyl or naphthalenyl and is positioned at either end or within the alkyl chain,
wherein when p=2, each R is independently C₆ to C₁₄ straight-chain alkyl; C₆ to C₁₄ straight-chain alkenyl; C₆ to C₁₄ branched alkyl; C₆ to C₁₄ branched alkenyl; C₉ to C₁₄ cycloalkyl-alkyl in which the cycloalkyl is C₃ to C₈ cycloalkyl positioned at the either end or within the alkyl chain; or C₈ to C₁₆ aryl-alkyl in which the aryl is phenyl or naphthalenyl and is positioned at either end or within the alkyl chain,
wherein when p=3, each R is independently C₆ to C₁₂ straight-chain alkyl; C₆ to C₁₂ straight-chain alkenyl; C₆ to C₁₂ branched alkyl; branched C₆ to C₁₂ alkenyl; C₉ to C₁₂ cycloalkyl-alkyl in which the cycloalkyl is C₃ to C cycloalkyl positioned at either end or within the alkyl chain; or Ca to C₁₄ aryl-alkyl in which the aryl is phenyl or naphthalenyl and is positioned at the either end or within the alkyl chain, and
wherein when p=4, each R is independently C₆ to C₁₀ straight-chain alkyl; straight-chain C₆ to C₁₀ alkenyl; C₆ to C₁₀ branched alkyl; C₆ to C₁₀ branched alkenyl; C₉ to C₁₀ cycloalkyl-alkyl in which the cycloalkyl is C₃ to C₈ cycloalkyl positioned at either end or within the alkyl; or C₈ to C₁₂ aryl-alky in which the aryl is phenyl or naphthalenyl and is positioned at the either end or within the alkyl chain.

In some embodiments, the ionizable cationic lipid has a structure of Formula 3,

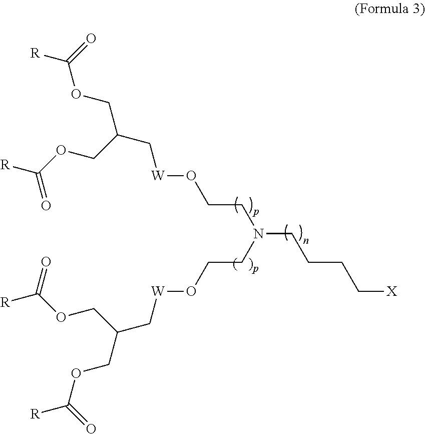

wherein W is C═O or CH₂,
n is an integer from 0 to 4,
X is

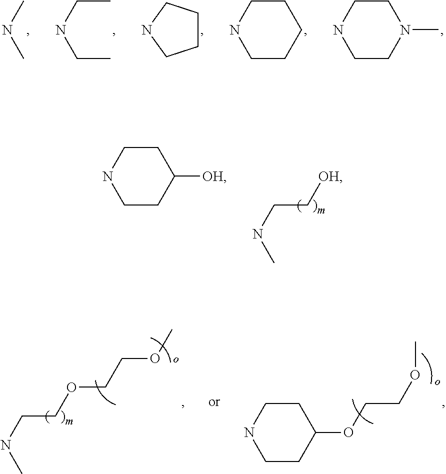

m is an integer from 1 to 3,
is an integer from 1 to 4,
p is an integer from 1 to 4,
wherein when p is 1, each R is independently C₈ to C₁₈ straight-chain alkyl; C₈ to C₁₈ straight-chain alkenyl; C₈ to C₁₈ branched alkyl; C₈ to C₁₈ branched alkenyl; C₁₁ to C₁₈ cycloalkyl-alkyl in which the cycloalkyl is C₃ to C₈ cycloalkyl positioned at either end or within the alkyl chain; or C₁₀ to C₂₀ aryl-alkyl in which the aryl is phenyl or naphthalenyl and is positioned at either end or within the alkyl chain,
wherein when p=2, each R is independently C₈ to C₁₆ straight-chain alkyl; C₈ to C₁₆ straight-chain alkenyl; C₈ to C₁₆ branched alkyl; C₈ to C₁₆ branched alkenyl; C₁₁ to C₁₆ cycloalkyl-alkyl in which the cycloalkyl is C₃ to C₈ cycloalkyl positioned at the either end or within the alkyl chain; or C₁₀ to C₁₈ aryl-alkyl in which the aryl is phenyl or naphthalenyl and is positioned at either end or within the alkyl chain,
wherein when p=3, each R is independently C₈ to C₁₄ straight-chain alkyl; C₈ to C₁₄ straight-chain alkenyl; C₈ to C₁₄ branched alkyl; C₈ to C₁₄ branched alkenyl; C₁₁ to C₁₄ cycloalkyl-alkyl in which the cycloalkyl is C₃ to C₈ cycloalkyl positioned at either end or within the alkyl chain; or C₁₀ to C₁₆ aryl-alkyl in which the aryl is phenyl or naphthalenyl and is positioned at the either end or within the alkyl chain, and
wherein when p=4, each R is independently C₈ to C₁₂ straight-chain alkyl; C₈ to C₁₂ straight-chain alkenyl; C₈ to C₁₂ branched alkyl; C₈ to C₁₂ branched alkenyl; C₁₁ to C₁₂ cycloalkyl-alkyl in which the cycloalkyl is C₃ to C₈ cycloalkyl positioned at either end or within the alkyl; or C₁₀ to C₁₄ aryl-alky in which the aryl is phenyl or naphthalenyl and is positioned at the either end or within the alkyl chain.

Ionizable cationic lipids of Formulae 1, 2, and 3 are more fully described in U.S. patent application Ser. No. 18/296,363 filed Apr. 5, 2023, which is incorporated by reference in its entirety.

With respect to the LNP or the tLNP, in various embodiments, the phospholipid comprises dioleoylphosphatidyl ethanolamine (DOPE), dimyristoylphosphatidyl choline (DMPC), distearoylphosphatidylcholine (DSPC), dimyristoylphosphatidyl glycerol (DMPG), dipalmitoyl phosphatidylcholine (DPPC), or 1,2-diarachidoyl-sn-glycero-3-phosphocholine (DAPC), or a combination thereof. Phospholipids contribute to formation of a membrane, whether monolayer or bilayer, surrounding the core of the LNP or tLNP. Additionally, phospholipids such as DSPC, DMPC, DPPC, DAPC impart stability and rigidity to membrane structure. phospholipids such as DOPE impart fusogenicity. Further phospholipids such as DMPG, which attains negative charge at physiologic pH, facilitates charge modulation. Thus, phospholipids constitute means for membrane formation, means for imparting membrane stability and rigidity, means for imparting fusogenicity, and means for charge modulation.

With respect to the LNP or the tLNP, in various embodiments, the sterol is cholesterol or a phytosterol. In further embodiments the phytosterol comprises campesterol, sitosterol, or stigmasterol, or combinations thereof. In preferred embodiments, the cholesterol is not animal-sourced but is obtained by synthesis using a plant sterol as a starting point. LNPs incorporating C-24 alkyl (such as methyl or ethyl) phytosterols have been reported to provide enhanced gene transfection. The length of the alkyl tail, the flexibility of the sterol ring, and polarity related to a retain C-3 —OH group are important to obtaining high transfection efficiency. While R-sitosterol and stigmasterol performed well, vitamin D2, D3 and calcipotriol, (analogs lacking intact body of cholesterol) and betulin, lupeol ursolic acid and olenolic acid (comprising a 5th ring) should be avoided. Sterols serve to fill space between other lipids in the LNP and influence LNP shape. Sterols also control fluidity of lipid compositions, reducing temperature dependence. Thus, sterols such as cholesterol, campesterol, fucosterol, β-sitosterol, and stigmasterol constitute means for controlling LNP shape and fluidity or sterol means for increasing transfection efficiency.

With respect to the LNP or the tLNP, in some embodiments, the co-lipid is absent or comprises an ionizable lipid, anionic or cationic. The co-lipid can be used to adjust any property of the LNP or tLNP such as surface charge, fluidity, rigidity, size, stability, etc. In some embodiments the ionizable lipid is cholesterol hemisuccinate (CHEMS). In some embodiments, the co-lipid is a charged lipid, such as a quaternary ammonium headgroup containing lipid. In some instances, the quaternary ammonium headgroup containing lipid comprises 1,2-dioleoyl-3-trimethylammonium propane (DOTAP), N-(1-(2,3-dioleyloxy)propyl)-N,N,N-trimethylammonium (DOTMA), or 3β-(N—(N′,N′-Dimethylaminoethane)carbamoyl)cholesterol (DC-Chol), or combinations thereof. These compounds are commonly provided as chloride, bromide, mesylate, or tosylate salts.

With respect to the LNP or the tLNP, in some embodiments, the PEG-lipid (that is, a lipid containing a polyethylene glycol moiety) is a C14-C20 lipid such as a C14, C15, C16, C17, C18, C19, or C20 lipid conjugated with a PEG. PEG-lipids with fatty acid chain lengths less than C14 are too rapidly lost from the (t)LNP while those with chain lengths greater than C20 are prone to difficulties with formulation. In some embodiments, the PEG is of 500-5000 Da molecular weight (MW) such as PEG-500, PEG-1000, PEG-1500, PEG-2000, PEG-2500, PEG-3000, PEG-3500, PEG-4000, PEG-4500, and PEG-5000. In some embodiments, the PEG unit has a MW of 2000 Da. In some instances, the MW2000 PEG-lipid comprises DMG-PEG2000 (1,2-dimyristoyl-rac-glycero-3-methoxypolyethylene glycol-2000), DPG-PEG2000 (1,2-dipalmitoyl-rac-glycero-3-methoxypolyethylene glycol-2000), or DSG-PEG2000 (1,2-distearoyl-rac-glycero-3-methoxypolyethylene glycol-2000), or combinations thereof. Alternatively, optically pure antipodes of the glycerol portion can be employed. They constitute means for preventing aggregation. In embodiments comprising both conjugated and unconjugated PEG-lipids, in some the conjugated and unconjugated PEG-lipid are the same and in others the PEG-lipid is different.

In some embodiments, the LNP comprises a symmetrical PEG-lipid that is a tri-ester PEG-lipid in which an esterified PEG moiety is attached to a central position on a scaffold and two identical fatty acids are esterified to two end positions on the scaffold. In some embodiments, the scaffold has the structure of Formula S1

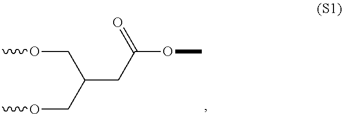

where [symbol] represents the points of esterification of the fatty acids and [symbol] represents the connection to the PEG moiety. In some embodiments, the fatty acid esters are C14-C20 straight-chain alkyl fatty acids. For example, the straight-chain alkyl fatty acid is C14, C15, C16, C17, C18, C19, or C20. In some embodiments, the PEG moiety is functionalized and the fatty acid esters are C16-C20 straight-chain alkyl fatty acids, e.g., C16, C17, C18, C19, or C20 straight-chain alkyl fatty acids. In some embodiments, the fatty acid esters are C14-C20 symmetric branched-chain alkyl fatty acids. For example, the branched-chain alkyl fatty acid is C14, C15, C16, C17, C18, C19, or C20. In some embodiments, the branch is at the 3, 4, 5, 6, or 7 position. In some embodiments, the LNP further comprises a symmetrical di-ester PEG-lipid newly disclosed herein. In some embodiments, the LNP further comprises an asymmetric PEG-lipid newly disclosed herein.

In some embodiments, the LNP comprises a symmetrical PEG-lipid that is a di-ester PEG-lipid in which a PEG-moiety is attached to a central position on a scaffold by an ether linkage and two identical fatty acids are esterified to two end positions on the scaffold. In some embodiments, the scaffold has the structure of Formula S2

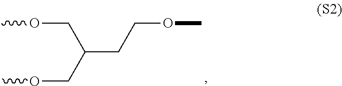

where [symbol] represents the points of esterification of the fatty acids and [symbol] represents the ether linkage to the PEG moiety, and which can be derived from scaffold of formula S2. In some embodiments, the fatty acid esters are C14-C20 straight-chain alkyl fatty acids. In some embodiments, the PEG moiety is functionalized and the fatty acid esters are C16-C20 straight-chain alkyl fatty acids, e.g., C16, C17, C18, C19, or C20 straight-chain alkyl fatty acids. In some embodiments, the fatty acid esters are C14-C20 symmetric branched-chain alkyl fatty acids. For example, the branched-chain alkyl fatty acid is C14, C15, C16, C17, C18, C19, or C20. In some embodiments, the branch is at the 3, 4, 5, 6, or 7 position. In some embodiments, the LNP further comprises a symmetrical tri-ester PEG-lipid, or a symmetrical di-ester PEG-lipid with a glycerol scaffold, newly disclosed herein. In some embodiments, the LNP further comprises an asymmetric PEG-lipid newly disclosed herein.

In some embodiments, the LNP comprises a symmetrical PEG-lipid that is a symmetrical di-ester PEG-lipid, in which a PEG-moiety is attached to a central position on a glycerol scaffold by an ether linkage and two identical fatty acids are esterified to two end positions on the glycerol scaffold. In some embodiments, the scaffold is a glycerol scaffold having the structure of Formula S3

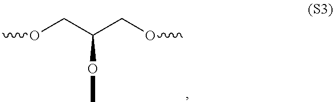

where [symbol] represents the points of esterification of the fatty acids and the [symbol] represents the ether linkage to the PEG moiety. In some embodiments, the fatty acid esters are C14-C20 straight-chain alkyl fatty acids. For example, the straight-chain alkyl fatty acid is C14, C15, C16, C17, C18, C19, or C20. In some embodiments, the PEG moiety is functionalized and the fatty acid esters are C16-C20 straight-chain alkyl fatty acids, e.g., C16, C17, C18, C19, or C20 straight-chain alkyl fatty acids. In some embodiments, the fatty acid esters are C14-C20 symmetric branched-chain alkyl. For example, the branched-chain alkyl fatty acid is C14, C15, C16, C17, C18, C19, or C20. In some embodiments, the branch is at the 3, 4, 5, 6, or 7 position. In some embodiments, the LNP further comprises a symmetrical tri-ester PEG-lipid, or a symmetrical di-ester PEG-lipid with a scaffold of formula S2, newly disclosed herein. In some embodiments, the LNP further comprises an asymmetric PEG-lipid newly disclosed herein.

In some embodiments, the LNP comprises an asymmetric glycerol-based PEG-lipid in which the glycerol scaffold has the structure of Formula S4

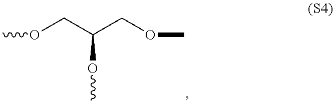

or the enantiomer or racemic mixture thereof, where [symbol] represents the points of esterification with a fatty acid, and [symbol] represents the point of ether formation with the PEG moiety, comprising two identical symmetrically branched fatty acids that each have a total carbon count of C14-C20. For example, the branched fatty acid is C14, C15, C16, C17, C18, C19, or C20. In some embodiments, the branch is at the 3, 4, 5, 6, or 7 position of the ester. In some embodiments, the LNP further comprises a symmetric PEG-lipid newly disclosed herein.

Any suitable chemistry may be used to conjugate the binding moiety to the PEG of the PEG-lipid, including maleimide (see Parhiz et al., Journal of Controlled Release 291:106-115, 2018) and click (see Kolb et al., Angewandte Chemie International Edition 40(11):2004-2021, 2001; and Evans, Australian Journal of Chemistry 60(6):384-395, 2007) chemistries. Reagents for such reactions include Lipid-PEG-maleimide, lipid-peg-cysteine, lipid-PEG-alkyne, and lipid-PEG-azide. If the binding moiety has been modified to comprise an alkyne or an azide group, then the PEG-lipid would carry with it either the azide or the alkyne necessary to participate in a click reaction. In some embodiments, instead of being functionalized with maleimide, azide, or alkyne, the PEG-lipid is functionalized with bromomaleimide, alkynylamide, or alkynylimide which also can form conjugates with an accessible sulfhydryl group in the binding moiety and provide more stable conjugations than maleimide.

PEG-lipids built on scaffolds S1, S2, S3, or S4, and bromomaleimide, alkynylamide, or alkynylimide functionalization and conjugation, are more fully described in POT Patent application PCT/US23/17648 filed Apr. 5, 2023, which is incorporated by reference in its entirety.

Particular compositions for precursors to tLNPs and tLNPs are disclosed in U.S. Provisional Patent application 63/505,424 filed May 31, 2023, 63/510,061 filed Jun. 23, 2023, and 63/520,303 filed Aug. 17, 2023, each of which is incorporated by reference in its entirety. LNP and tLNP compositions can include those of Table 1. In various embodiments, N/P can be from 3 to 9 or any integer-bound sub-range in that range or about any integer in that range

TABLE 1
LNP Compositions

| Composition | | |
|---|---|---|
| Code | lipid composition [ratios] | N/P |
| BF1 | ALC-0315:DSPC:CHOL:DMG-PEG(2k):DSPE-PEG(2k)-MAL | 6 |
| | [50:10:38.5:1.4:0.1] | |
| F1 | CICL1:DSPC:CHOL:DMG-PEG(2k):DSPE-PEG(2k)-MAL | 6 |
| | [50:10:38.5:1.4:0.1] | |
| F2 | CICL1:DSPC:CHOL:DMG-PEG(2k):DSPE-PEG(2k)-MAL | 3 |
| | [50:10:38.5:1.3:0.2] | |
| F3 | CICL1:DSPC:CHOL:DMG-PEG(2k):DSPE-PEG(2k)-MAL | 9 |
| | [50:10:38.5:1.425:0.075] | |
| F4 | CICL1:DSPC:CHOL:DMG-PEG(2k):DSPE-PEG(2k)-MAL | 6 |
| | [42:10:46.5:1.4:0.1] | |
| F5 | CICL1:DSPC:CHOL:DMG-PEG(2k):DSPE-PEG(2k)-MAL | 6 |
| | [58:10:30.5:1.4:0.1] | |
| F6 | CICL1:DSPC:CHOL:DSG-PEG(2k):DSPE-PEG(2k)-MAL | 6 |
| | [35:10:53.5:1.4:0.1] | |
| F7 | CICL1:DSPC:CHOL:DSG-PEG(2k):DSPE-PEG(2k)-MAL | 6 |
| | [42:10:46.5:1.4:0.1] | |
| F8 | CICL1:DSPC:CHOL:DSG-PEG(2k):DSPE-PEG(2k)-MAL | 6 |
| | [50:10:38.5:1.4:0.1] | |
| F9 | CICL1:DSPC:CHOL:DSG-PEG(2k):DSPE-PEG(2k)-MAL | 6 |
| | [58:10:30.5:1.4:0.1] | |
| F10 | CICL1:DSPC:CHOL:DSG-PEG(2k):DSPE-PEG(2k)-MAL | 6 |
| | [62:10:26.5:1.4:0.1] | |
| F11 | CICL1:DSPC:CHOL:DMG-PEG(2k):DSPE-PEG(2k)-MAL | 6 |
| | [58:7:33.5:1.4:0.1] | |
| F12 | CICL1:DSPC:CHOL:DPG-PEG(2k):DSPE-PEG(2k)-MAL | 6 |
| | [58:7:33.5:1.4:0.1] | |
| F13 | CICL1:DSPC:CHOL:DSG-PEG(2k):DSPE-PEG(2k)-MAL | 6 |
| | [58:7:33.5:1.4:0.1] | |
| F14 | CICL1:DSPC:CHOL:DMG-PEG(2k):DSPE-PEG(2k)-MAL | 6 |
| | [58:7:34:0.9:0.1] | |
| F15 | CICL1:DSPC:CHOL:DSG-PEG(2k):DSPE-PEG(2k)-MAL | 6 |
| | [58:10:30:1.9:0.1] | |
| F16 | CICL1:DSPC:CHOL:DSG-PEG(2k):DSPE-PEG(2k)-MAL | 6 |
| | [50:10:39.5:0.4:0.1] | |
| F17 | CICL1:DSPC:CHOL:DSG-PEG(2k):DSPE-PEG(2k)-MAL | 6 |
| | [50:10:39:0.9:0.1] | |
| F18 | CICL1:DSPC:CHOL:DSG-PEG(2k):DSPE-PEG(2k)-MAL | 6 |
| | [50:10:38.5:1.4:0.1] | |
| F19 | CICL1:DSPC:CHOL:DSG-PEG(2k):DSPE-PEG(2k)-MAL | 6 |
| | [50:10:38:1.9:0.1] | |
| F20 | CICL1:DSPC:CHOL:DSG-PEG(2k):DSPE-PEG(2k)-MAL | 6 |
| | [50:10:37.5:2.4:0.1] | |
| F21 | CICL1:DSPC:CHOL:DSG-PEG(2k):DSPE-PEG(2k)-MAL | 6 |
| | [50:10:37:2.9:0.1] | |
| F22 | CICL1:DSPC:CHOL:DSG-PEG(2k):DSPE-PEG(2k)-MAL | 6 |
| | [58:10:31:0.9:0.1] | |
| F23 | CICL1:DSPC:CHOL:DSG-PEG(2k):DSPE-PEG(2k)-MAL | 6 |
| | [58:10:30:1.9:0.1] | |
| F24 | CICL1:DSPC:CHOL:DSG-PEG(2k):DSPE-PEG(2k)-MAL | 6 |
| | [58:10:29.5:2.4:0.1] | |
| F25 | CICL1:DSPC:CHOL:DSPE-PEG(0.75k):DSPE-PEG(2k)-MAL | 6 |
| | [58:10:30.5:1.4:0.1] | |
| F26 | CICL1:DSPC:CHOL:DSPE-PEG(1k):DSPE-PEG(2k)-MAL | 6 |
| | [58:10:30.5:1.4:0.1] | |
| F27 | CICL1:DSPC:CHOL:DMPE-PEG(1k):DSPE-PEG(2k)-MAL | 6 |
| | [58:10:30.5:1.4:0.1] | |
| F29 | CICL1:DSPC:CHOL:DSG-PEG(5k):DSPE-PEG(5k)-MAL | 6 |
| | [58:10:31.4:0.5:0.1] | |
| F30 | CICL1:DSPC:CHOL:DMG-PEG(2k):DSG-PEG(2k)-MAL | 6 |
| | [58:10:30.5:1.4:0.1] | |
| F31 | CICL1:DSPC:CHOL:DSG-PEG(2k):DSG-PEG(2k)-MAL | 6 |
| | [58:10:30.5:1.4:0.1] | |
| F32 | CICL1:DSPC:CHOL:DSPE-PEG(2k):DSPE-PEG(2k)-MAL | 6 |
| | [58:10:30.5:1.4:0.1] | |
| F33 | CICL1:DSPC:CHOL:DSG-PEG(2k):DSPE-PEG(5k)-MAL | 6 |
| | [58:10:30.5:1.4:0.1] | |
| F34 | CICL1:DSPC:CHOL:DSG-PEG(2k):DSPE-PEG(2k)-MAL | 6 |
| | [58:13:27.5:1.4:0.1] | |
| F35 | CICL1:DMPC:CHOL:DSG-PEG(2k):DSPE-PEG(2k)-MAL | 6 |
| | [58:10:30.5:1.4:0.1] | |
| F36 | CICL1:DPPC:CHOL:DSG-PEG(2k):DSPE-PEG(2k)-MAL | 6 |
| | [58:10:30.5:1.4:0.1] | |
| F37 | CICL1:DAPC:CHOL:DSG-PEG(2k):DSPE-PEG(2k)-MAL | 6 |
| | [58:10:30.5:1.4:0.1] | |
| F38 | CICL1:18:1PA:CHOL:DSG-PEG(2k):DSPE-PEG(2k)-MAL | 6 |
| | [58:10:30.5:1.4:0.1] | |
| F40 | CICL1:DSPC:CHOL:20(S)-Hydroxycholesterol:DSG- | 6 |
| | PEG(2k):DSPE-PEG(2k)-MAL [58:10:22.9:7.6:1.4:0.1] | |
| F41 | CICL1:DSPC:CHOL:β-Sitosterol:DSG-PEG(2k):DSPE-PEG(2k)- | 6 |
| | MAL [58:10:22.9:7.6:1.4:0.1] | |
| F42 | CICL1:DSPC:CHOL:β-Sitosterol:DSG-PEG(2k):DSPE-PEG(2k)- | 6 |
| | MAL [58:10:15.25:15.25:1.4:0.1] | |

Certain aspects include a method of making a tLNP comprising rapid mixing of an aqueous solution of a nucleic acid encoding a BRM and an alcoholic solution of the lipids. A variety of appropriate mixers are known in the art including multi-inlet vortex mixers and impingement jet mixers. In some embodiments, the lipid mixture includes functionalized PEG-lipid, for later conjugation to a targeting moiety. In other embodiments, the functionalized PEG-lipid is inserted into and LNP subsequent to initial formation of an LNP from other components. In either type of embodiment, the targeting moiety is conjugated to functionalized PEG-lipid after the functionalized PEG-lipid containing LNP is formed. Protocols for conjugation can be found, for example, in Parhiz et al. J. Controlled Release 291:106-115, 2018, and Tombacz et al., Molecular Therapy 29(11):3293-3304, 2021, each of which is incorporated by reference for all that it teaches about conjugation of PEG-lipids to binding moieties that is not inconsistent with the present disclosure.

After the LNP are formed they are diluted with buffer, for example phosphate, HEPES, or Tris, in a pH range of 6 to 8.5 to reduce the alcohol (ethanol) concentration, The diluted LNP are purified either by dialysis or ultrafiltration or diafiltration using tangential flow filtration (TFF) against a buffer in a pH range of 6 to 8.5 (for example, phosphate, HEPES, or Tris) to remove the alcohol. Alternatively, one can use size exclusion chromatography. Once the alcohol is completely removed the buffer is exchanged with like buffer containing a cryoprotectant (for example, glycerol or a sugar such as sucrose, trehalose, or mannose). The LNP are concentrated to a desired concentrated, followed by 0.2 μm filtration through, for example, a polyethersulfone (PES) filter and filled into glass vials, stoppered, capped, and stored frozen. In alternative embodiments, a lyoprotectant is used and the LNP lyophilized for storage instead of as a frozen liquid. Further methodologies for making LNP can be found, for example, in US20200297634, US20130115274, and WO2017/048770, each of which is incorporated by reference for all that it teaches about the production of LNP that is not inconsistent with the present disclosure.

In various embodiments, the binding moiety of the tNP comprises an antigen binding domain of an antibody, an antigen, a ligand-binding domain of a receptor, or a receptor ligand. In some embodiments, the binding moiety comprising an antigen binding domain of an antibody comprises a complete antibody, an F(ab)2, an Fab, a minibody, a single-chain Fv (scFv), a diabody, a VH domain, or a nanobody, such as a VHH or single domain antibody. In some embodiments, a complete antibody has a modified Fc region to reduce or eliminate secondary functions, such as antibody-dependent cellular cytotoxicity (ADCC), antibody-dependent cellular phagocytosis (ADCP), and/or complement-dependent cytotoxicity (CDC). In some embodiments, binding moieties having more than one specificity are used such as bispecific or multispecific binders. In some embodiments, receptor ligand is a peptide. In some embodiments, the receptor ligand is a carbohydrate, for example, a carbohydrate comprising terminal galactose or N-acetylgalactosamine units, which are bound by the asialoglycoprotein receptor. These binding moieties constitute means for NP targeting. In some embodiments, the binding moiety is a polypeptide comprising a binding domain and an N- or C-terminal extension comprising an accessible thiol group for use in conjugation with a functionalized nanoparticle. Alternatively, one can add an alkyne or an azide to a sulfhydryl or an epsilon amino of a lysine to participate in a click chemistry reaction for a functionalized nanoparticle. In another alternative, an epsilon amino of a lysine can be reacted with N-succinimidyl S-acetylthioacetate (SATA) to introduce a reactive sulfhydryl group which can then be reacted with a maleimide-modified nanoparticle, for example, a nanoparticle comprising a maleimide-modified PEG-lipid, to form the conjugate. Some embodiments specifically include one or more of these binding moieties. Other embodiments specifically exclude one or more of these binding moieties.

In some embodiments, the binding moiety of a tLNP comprises an antibody or an antigen-binding portion thereof. The term “antibody” may refer to a protein comprising an immunoglobulin domain having hypervariable regions determining the specificity with which the antibody binds antigen; so-called complementarity determining regions (CDRs). The term antibody can thus refer to intact or whole antibodies as well as antibody fragments and constructs comprising an antigen binding portion of a whole antibody. While the canonical natural antibody has a pair of heavy and light chains, camelids (camels, alpacas, llamas, etc.) produce antibodies with both the canonical structure and antibodies comprising only heavy chains. The variable region of the camelid heavy chain only antibody has a distinct structure with a lengthened CDR3 referred to as VHH or, when produced as a fragment, a nanobody.

The term antibody may include natural antibodies or genetically engineered or otherwise modified forms of immunoglobulins or portions thereof, including chimeric antibodies, humanized antibodies, human antibodies, or synthetic antibodies. The antibodies may be monoclonal or polyclonal antibodies. The term “monoclonal antibody” arose out of hybridoma technology but is now used to refer to any singular molecular species of antibody regardless of how it was originated or produced. In those embodiments wherein an antibody comprises an antigen-binding portion of an immunoglobulin molecule, the antibody may include, but is not limited to, a single chain variable fragment antibody (scFv), a disulfide linked Fv, a single domain antibody (sdAb), a VHH antibody, an antigen-binding fragment (Fab), a Fab′ fragment, a F(ab′)2 fragment, or a diabody. Specifically, an scFv antibody can be derived from a natural antibody by linking the variable regions of the heavy (V_{H}) and light (V_{L}) chains of the immunoglobulin with a short linker peptide. Similarly, a disulfide linked Fv antibody can be generated by linking the VH and VL using an interdomain disulfide bond. On the other hand, sdAbs consist of only the variable region from either the heavy or light chain and usually are the smallest antigen-binding fragments of antibodies. A VHH antibody is the antigen-binding fragment of heavy chain only. The term “antigen-binding portion” may refer to a portion of an antibody as described that possesses the ability to specifically recognize, associate, unite, or combine with a target molecule. An antigen-binding portion includes any naturally occurring, synthetic, semi-synthetic, or recombinantly produced binding partner for a specific antigen.

Antibodies can be obtained through immunization, selection from a naïve or immunized library (for example, by phage display), alteration of an isolated antibody-encoding sequence, or any combination thereof. Antibody variable regions can be those arising from the germ line of a particular species, or they can be chimeric, containing segments of multiple species possibly further altered to optimize characteristics such as binding affinity or low immunogenicity. For treating humans, it is desirable that the antibody have a human sequence. If a human antibody of the desired specificity is not available, but such an antibody from a non-human species is, the non-human antibody can be humanized, for example, through CDR grafting, in which the CDRs from the non-human antibody are placed into the respective positions in a framework of a compatible human antibody by engineering the encoding DNA. Similar considerations and procedures can be applied mutandis mutatis to antibodies for treating other species. Antibodies and their antigen binding domains may be used variously as or as part of the targeting moiety for a tLNP for delivering an engineering agent or a nucleic acid encoded conditioning agent, a conditioning agent, or a reprogramming agent (such as CAR and immune cell engagers).

Thus, antibodies and antigen-binding portions thereof constitute means for binding to the surface antigen on the immune cell, means for altering signal transduction by the surface antigen, means for promotes transcription and/or translation of the internal payload, and/or means for conditioning the immune cell. Table 2 provides exemplary embodiments of the antibodies or antigen-binding portions thereof described herein. The antibodies provided in Table 2 can be modified to be any form of an antibody as described above, including, for example, scFv, minibodies, Fab, Fab2, diabodies, scFv, and VHH. The antibodies of Table 2 are exemplary antibodies that constitute means for binding to a surface antigen of an immune cell. The sequences in Table 2 include VH variable domains underlined and VL variable domains in bold.

TABLE 2
Exemplary Embodiments of Antibodies or Antigen-binding Portions

| | | Heavy | Light | | |
|---|---|---|---|---|---|
| | | Chain | Chain | | |
| | | SEQ | SEQ | | |
| | Antibody | ID | ID | | |
| Target | Name | NO: | NO: | Heavy Chain (V_{H}) (V_{L}) | Light Chain (V_{L}) |
| BCMA | erlana- | 30 | 31 | EVQLLESGGGLVQPGGSLRLSCAASGFTFSS | EIVLTQSPGTLSLSPG |
| | tamab | | | YPMSWVRQAPGKGLEWVSAIGGSGGSLPYA | ERATLSCRASQSVSSS |
| | | | | DIVKGRFTISRDNSKNTLYLQMNSLRAEDTAV | YLAWYQQKPGQAPRLL |
| | | | | YYCARYWPMDIWGQGTLVTVSSASTKGPSVF | MYDASIRATGIPDRFS |
| | | | | PLAPCSRSTSESTAALGCLVKDYFPEPVTVSW | GSGSGTDFTLTISRLE |
| | | | | NSGALTSGVHTFPAVLQSSGLYSLSSVVTVPS | PEDFAVYYCQQYQSWP |
| | | | | SNFGTQTYTCNVDHKPSNTKVDKTVERKCEV | LTFGQGTKVEIKRTVA |
| | | | | ECPECPAPPVAGPSVFLFPPKPKDTLMISRTP | APSVFIFPPSDEQLKS |
| | | | | EVTCVVVAVSHEDPEVQFNWYVDGVEVHNAK | GTASVVCLLNNFYPRE |
| | | | | TKPREEQFNSTFRVVSVLTVVHQDWLNGKEY | AKVQWKVDNALQSGNS |
| | | | | KCKVSNKGLPSSIEKTISKTKGQPREPQVYTL | QESVTEQDSKDSTYSL |
| | | | | PPSREEMTKNQVSLTCEVKGFYPSDIAVEWES | SSTLTLSKADYEKHKV |
| | | | | NGQPENNYKTTPPMLDSDGSFFLYSKLTVDK | YACEVTHQGLSSPVTK |
| | | | | SRWQQGNVFSCSVMHEALHNHYTQKSLSLSP | SFNRGEC |
| | | | | GK | |
| BCMA | Alnuc- | 32 | 33 | EVQLLESGGGLVQPGGSLRLSCAASGFTFSD | EIVLTQSPGTLSLSPG |
| | tamab- | | | NAMGWVRQAPGKGLEWVSAISGPGSSTYYA | ERATLSCRASQSVSDE |
| | First | | | DSVKGRFTISRDNSKNTLYLQMNSLRAEDTAV | YLSWYQQKPGQAPRLL |
| | heavy | | | YYCAKVLGWFDYWGQGTLVTVSSASTKGPSV | IHSASTRATGIPDRFS |
| | chain | | | FPLAPSSKSTSGGTAALGCLVEDYFPEPVTVS | GSGSGTDFTLAISRLE |
| | and | | | WNSGALTSGVHTFPAVLQSSGLYSLSSVVTVP | PEDFAVYYCQQYGYPP |
| | first | | | SSSLGTQTYICNVNHKPSNTKVDEKVEPKSCD | DFTFGQGTKVEIKRTV |
| | light | | | KTHTCPPCPAPEAAGGPSVFLFPPKPKDTLMI | AAPSVFIFPPSDRKLK |
| | chain | | | SRTPEVTCVVVDVSHEDPEVKFNWYVDGVEV | SGTASVVCLLNNFYPR |
| | | | | HNAKTKPREEQYNSTYRVVSVLTVLHQDWLN | EAKVQWKVDNALQSGN |
| | | | | GKEYKCKVSNKALGAPIEKTISKAKGQPREPQ | SQESVTEQDSKDSTYS |
| | | | | VCTLPPSRDELTKNQVSLSCAVKGFYPSDIAV | LSSTLTLSKADYEKHK |
| | | | | EWESNGQPENNYKTTPPVLDSDGSFFLVSKL | VYACEVTHQGLSSPVT |
| | | | | TVDKSRWQQGNVFSCSVMHEALHNHYTQKS | KSFNRGEC |
| | | | | LSLSPGK | |
| BCMA | Alnuc- | 34 | | EVQLLESGGGLVQPGGSLRLSCAASGFTFSD | |
| | tamab- | | | NAMGWVRQAPGKGLEWVSAISGPGSSTYYA | |
| | Second | | | DSVKGRFTISRDNSKNTLYLQMNSLRAEDTAV | |
| | heavy | | | YYCAKVLGWFDYWGQGTLVTVSSASTKGPSV | |
| | chain | | | FPLAPSSKSTSGGTAALGCLVEDYFPEPVTVS | |
| | | | | WNSGALTSGVHTFPAVLQSSGLYSLSSVVTVP | |
| | | | | SSSLGTQTYICNVNHKPSNTKVDEKVEPKSCD | |
| | | | | KTHTCPPCPAPEAAGGPSVFLFPPKPKDTLMI | |
| | | | | SRTPEVTCVVVDVSHEDPEVKFNWYVDGVEV | |
| | | | | HNAKTKPREEQYNSTYRVVSVLTVLHQDWLN | |
| | | | | GKEYKCKVSNKALGAPIEKTISKAKGQPREPQ | |
| | | | | VCTLPPSRDELTKNQVSLSCAVKGFYPSDIAV | |
| | | | | EWESNGQPENNYKTTPPVLDSDGSFFLVSKL | |
| | | | | TVDKSRWQQGNVFSCSVMHEALHNHYTQKS | |
| | | | | LSLSPGK | |
| BCMA | Linvo- | 35 | | EVQLVESGGGLVQPGGSLRLSCAASGFTFSN | |
| | selta- | | | FWMTWVRQAPGKGLEWVANMNQDGSEKYY | |
| | mab- | | | VDSVKGRFTISRDNAKSSLYLQMNSLRAEDTA | |
| | bi- | | | VYYCARDREYCISTSCYDDFDYWGQGTLVTV | |
| | specific | | | SSASTKGPSVFPLAPCSRSTSESTAALGCLVK | |
| | anti- | | | DYFPEPVTVSWNSGALTSGVHTFPAVLQSSG | |
| | | | | LYSLSSVVTVPSSSLGTKTYTCNVDHKPSNTK | |
| | | | | VDKRVESKYGPPCPPCPAPPVAGPSVFLFPP | |
| | CD3E | | | KPKDTLMISRTPEVTCVVVDVSQEDPEVQFN | |
| | and anti- | | | WYVDGVEVHNAKTKPREEQFNSTYRVVSVLT | |
| | BCMA | | | VLHQDWLNGKEYKCKVSNKGLPSSIEKTISKA | |
| | antibody | | | KGQPREPQVYTLPPSQEEMTKNQVSLTCLVK | |
| | | | | GFYPSDIAVEWESNGQPENNYKTTPPVLDSD | |
| | | | | GSFFLYSRLTVDKSRWQEGNVFSCSVMHEAL | |
| | | | | HNHYTQKSLSLSLGK | |
| BCMA | teclis- | 36 | 37 | QLQLQESGPGLVKPSETLSLTCTVSGGSISSG | SYVLTQPPSVSVAPGQ |
| | tamab | | | SYFWGWIRQPPGKGLEWIGSIYYSGITYYNPS | TARITCGGNNIGSKSV |
| | | | | LKSRVTISVDTSKNQFSLKLSSVTAADTAVYY | HWYQQPPGQAPVVVVY |
| | | | | CARHDGAVAGLFDYWGQGTLVTVSSASTKGPS | DDSDRPSGIPERFSGS |
| | | | | VFPLAPCSRSTSESTAALGCLVKDYFPEPVTV | NSGNTATLTISRVEAG |
| | | | | SWNSGALTSGVHTFPAVLQSSGLYSLSSVVTV | DEAVYYCQVWDSSSDH |
| | | | | PSSSLGTKTYTCNVDHKPSNTKVDKRVESKY | VVFGGGTKLTVLGQPK |
| | | | | GPPCPPCPAPEAAGGPSVFLFPPKPKDTLMIS | AAPSVTLFPPSSEELQ |
| | | | | RTPEVTCVVVDVSQEDPEVQFNWYVDGVEVH | ANKATLVCLISDFYPG |
| | | | | NAKTKPREEQFNSTYRVVSVLTVLHQDWLNG | AVTVAWKGDSSPVKAG |
| | | | | KEYKCKVSNKGLPSSIEKTISKAKGQPREPQV | VETTTPSKQSNNKYAA |
| | | | | YTLPPSQEEMTKNQVSLTCLVKGFYPSDIAVE | SSYLSLTPEQWKSHRS |
| | | | | WESNGQPENNYKTTPPVLDSDGSFFLYSRLT | YSCQVTHEGSTVEKTV |
| | | | | VDKSRWQEGNVFSCSVMHEALHNHYTQKSL | APTECS |
| | | | | SLSLGK | |
| BCMA | belantamab | 38 | 39 | QVQLVQSGAEVKKPGSSVKVSCKASGGTFSN | DIQMTQSPSSLSASVG |
| | | | | YWMHWVRQAPGQGLEWMGATYRGHSDTYY | DRVTITCSASQDISNY |
| | | | | NQKFKGRVTITADKSTSTAYMELSSLRSEDTA | LNWYQQKPGKAPKLLI |
| | | | | VYYCARGAIYDGYDVLDNWGQGTLVTVSSAS | YYTSNLHSGVPSRFSG |
| | | | | TKGPSVFPLAPSSKSTSGGTAALGCLVKDYFP | SGSGTDFTLTISSLQP |
| | | | | EPVTVSWNSGALTSGVHTFPAVLQSSGLYSLS | EDFATYYCQQYRKLPW |
| | | | | SVVTVPSSSLGTQTYICNVNHKPSNTKVDKKV | TFGQGTKLEIKRTVAA |
| | | | | EPKSCDKTHTCPPCPAPELLGGPSVFLFPPKP | PSVFIFPPSDEQLKSG |
| | | | | KDTLMISRTPEVTCVVVDVSHEDPEVKENWYV | TASVVCLLNNFYPREA |
| | | | | DGVEVHNAKTKPREEQYNSTYRVVSVLTVLH | KVQWKVDNALQSGNSQ |
| | | | | QDWLNGKEYKCKVSNKALPAPIEKTISKAKGQ | ESVTEQDSKDSTYSLS |
| | | | | PREPQVYTLPPSRDELTKNQVSLTCLVKGFYP | STLTLSKADYEKHKVY |
| | | | | SDIAVEWESNGQPENNYKTTPPVLDSDGSFFL | ACEVTHQGLSSPVTKS |
| | | | | YSKLTVDKSRWQQGNVFSCSVMHEALHNHYT | FNRGEC |
| | | | | QKSLSLSPGK | |
| BCMA | Pavuru- | 40 | | QVQLVQSGAEVKKPGASVKVSCKASGYTFTN | |
| | tamab- | | | HIIHWVRQAPGQCLEWMGYINPYPGYHAYNE | |
| | BiTE with | | | KFQGRATMTSDTSTSTVYMELSSLRSEDTAVY | |
| | speci- | | | YCARDGYYRDTDVLDYWGQGTLVTVSSGGG | |
| | ficity | | | GSGGGGSGGGGSDIQMTQSPSSLSASVGDR | |
| | against | | | VTITCQASQDISNYLNWYQQKPGKAPKLLIYY | |
| | BCMA | | | TSRLHTGVPSRFSGSGSGTDFTFTISSLEPED | |
| | and CD3 | | | IATYYCQQGNTLPWTFGCGTKVEIKSGGGGS | |
| | | | | EVQLVESGGGLVQPGGSLKLSCAASGFTFNK | |
| | | | | YAMNWVRQAPGKGLEWVARIRSKYNNYATYY | |
| | | | | ADSVKDRFTISRDDSKNTAYLQMNNLKTEDTA | |
| | | | | VYYCVRHGNFGNSYISYWAYWGQGTLVTVSS | |
| | | | | GGGGSGGGGSGGGGSQTVVTQEPSLTVSPG | |
| | | | | GTVTLTCGSSTGAVTSGNYPNWVQQKPGQA | |
| | | | | PRGLIGGTKFLAPGTPARFSGSLLGGKAALTL | |
| | | | | SGVQPEDEAEYYCVLWYSNRWVFGGGTKLT | |
| | | | | VLGGGGDKTHTCPPCPAPELLGGPSVFLFPP | |
| | | | | KPKDTLMISRTPEVTCVVVDVSHEDPEVKFNW | |
| | | | | YVDGVEVHNAKTKPCEEQYGSTYRCVSVLTV | |
| | | | | LHQDWLNGKEYKCKVSNKALPAPIEKTISKAK | |
| | | | | GQPREPQVYTLPPSREEMTKNQVSLTCLVKG | |
| | | | | FYPSDIAVEWESNGQPENNYKTTPPVLDSDG | |
| | | | | SFFLYSKLTVDKSRWQQGNVFSCSVMHEALH | |
| | | | | NHYTQKSLSLSPGKGGGGSGGGGGGGGSG | |
| | | | | GGGSGGGGSGGGGSDKTHTCPPCPAPELLG | |
| | | | | GPSVFLFPPKPKDTLMISRTPEVTCVVVDVSH | |
| | | | | EDPEVKFNWYVDGVEVHNAKTKPCEEQYGST | |
| | | | | YRCVSVLTVLHQDWLNGKEYKCKVSNKALPA | |
| | | | | PIEKTISKAKGQPREPQVYTLPPSREEMTKNQ | |
| | | | | VSLTCLVKGFYPSDIAVEWESNGQPENNYKTT | |
| | | | | PPVLDSDGSFFLYSKLTVDKSRWQQGNVFSC | |
| | | | | SVMHEALHNHYTQKSLSLSPGK | |
| BCMA | Pacanalo- | 41 | | QVQLVQSGAEVKKPGASVKVSCKASGYTFTN | |
| | tabab- | | | HIIHWVRQAPGQGLEWMGYINPYPGYHAYNE | |
| | BiTE with | | | KFQGRATMTSDTSTSTVYMELSSLRSEDTAVY | |
| | affinity | | | YCARDGYYRDTDVLDYWGQGTLVTVSSGGG | |
| | against | | | GSGGGGSGGGGSDIQMTQSPSSLSASVGDR | |
| | BCMA and | | | VTITCQASQDISNYLNWYQQKPGKAPKLLIYY | |
| | CD3E | | | TSRLHTGVPSRFSGSGSGTDFTFTISSLEPED | |
| | | | | IATYYCQQGNTLPWTFGQGTKVEIKSGGGGS | |
| | | | | EVQLVESGGGLVQPGGSLKLSCAASGFTFNK | |
| | | | | YAMNWVRQAPGKGLEWVARIRSKYNNYATYY | |
| | | | | ADSVKDRFTISRDDSKNTAYLQMNNLKTEDTA | |
| | | | | VYYCVRHGNFGNSYISYWAYWGQGTLVTVSS | |
| | | | | GGGGSGGGGSGGGGSQTVVTQEPSLTVSPG | |
| | | | | GTVTLTCGSSTGAVTSGNYPNWVQQKPGQA | |
| | | | | PRGLIGGTKFLAPGTPARFSGSLLGGKAALTL | |
| | | | | SGVQPEDEAEYYCVLWYSNRWVFGGGTKLT | |
| | | | | VLHH | |
| BCMA | hSG16.17_V | 42 | 43 | QVQLVQSGAEVKKPGASVKLSCKASGYTFTD | DIQMTQSPSSVSASVG |
| | H3/VK2- | | | YYIHWVRQAPGQGLEWIGYINPNSGYTNYAQ | DRVTITCLASEDISDD |
| | BCMA | | | KFQGRATMTADKSINTAYVELSRLRSDDTAVY | LAWYQQKPGKAPKVLV |
| | binding | | | FCTRYMWERVTGFFDFWGQGTMVTVSS | YTTSSLQSGVPSRFSG |
| | fragment | | | | SGSGTDFTLTISSLQP |
| | | | | | EDFATYFCQQTYKFPP |
| | | | | | TFGGGTKVEIK |
| BCMA | hSG16.45_V | 44 | 45 | EVQLVESGGGLVQPGGSLRLSCAASGFTFND | EIVLTQSPGTLSLSPG |
| | H5/VK2 B- | | | HWMTWVRQAPGKGLVWVSSITNTGGATYYA | ERATLSCRASSSVSVM |
| | BCMA | | | DSVKGRFTISRDNAKNTLYLQMNSLRAEDTAV | YWYQQKPGQAPRLLIY |
| | binding | | | YYCTSPGLYFDYWGQGTMVTVSS | STSSLASGIPDRFSGS |
| | fragment | | | | GSGTDFTLTISRLEPE |
| | | | | | DFAVYYCHQWSSDPPT |
| | | | | | FGQGTKLEIK |
| BTLA | icatolimab | 46 | 47 | QVQLVQSGAEVKKPGASVKLSCKASGYNFKH | DVVMTQTPLSLSVTPG |
| | | | | TYAHWVRQAPGQGLEWIGRIDPANGNTKYDP | QPASISCKSSQSLLDS |
| | | | | KFQGRATMTADTASNTAYLELSSLRSEDTAVY | DGKTYLNWFQQRPGQS |
| | | | | YCVADHYGSSLLDYWGQGTLVTVSSASTKGP | PRRLIYLVSKLDSGVP |
| | | | | SVFPLAPCSRSTSESTAALGCLVKDYFPEPVT | DRFSGSGSGTDFTLKI |
| | | | | VSWNSGALTSGVHTFPAVLQSSGLYSLSSVVT | SRVEAEDVGVYYCWQG |
| | | | | VPSSSLGTKTYTCNVDHKPSNTKVDKRVESKY | TYFPYTFGQGTKLEIK |
| | | | | GPPCPPCPAPEFLGGPSVFLFPPKPKDTLMIS | RTVAAPSVFIFPPSDE |
| | | | | RTPEVTCVVVDVSQEDPEVQFNWYVDGVEVH | QLKSGTASVVCLLNNF |
| | | | | NAKTKPREEQFNSTYRVVSVLTVLHQDWLNG | YPREAKVQWKVDNALQ |
| | | | | KEYKCKVSNKGLPSSIEKTISKAKGQPREPQV | SGNSQESVTEQDSKDS |
| | | | | YTLPPSQEEMTKNQVSLTCLVKGFYPSDIAVE | TYSLSSTLTLSKADYE |
| | | | | WESNGQPENNYKTTPPVLDSDGSFFLYSRLT | KHKVYACEVTHQGLSS |
| | | | | VDKSRWQEGNVFSCSVMHEALHNHYTQKSL | PVTKSFNRGEC |
| | | | | SLSLGK | |
| CD117 | barzolvo- | 48 | 49 | QVQLVQSGAEVKKPGASVKLSCKASGYTFTD | DIVMTQSPSSLSASVG |
| | limab | | | YYINWVRQAPGKGLEWIARIYPGSGNTYYNEK | DRVTITCKASQNVRTN |
| | | | | FKGRATLTADKSTSTAYMQLSSLRSEDTAVYF | VAWYQQKPGKAPKALI |
| | | | | CARGVYYFDYWGQGTTVTVSSASTKGPSVFP | YSASYRYSGVPDRFTG |
| | | | | LAPSSKSTSGGTAALGCLVKDYFPEPVTVSW | SGSGTDFTLTISSLQP |
| | | | | NSGALTSGVHTFPAVLQSSGLYSLSSVVTVPS | EDFADYFCQQYNSYPR |
| | | | | SSLGTQTYICNVNHKPSNTKVDKKVEPKSCDK | TFGGGTKVEIKRTVAA |
| | | | | THTCPPCPAPEAQGGPSVFLFPPKPKDTLYIT | PSVFIFPPSDEQLKSG |
| | | | | REPEVTCVVVDVSHEDPEVKFNWYVDGVEVH | TASVVCLLNNFYPREA |
| | | | | NAKTKPREEQYNSTYRVVSVLTVLHQDWLNG | KVQWKVDNALQSGNSQ |
| | | | | KEYKCQVSNKALPAPIEKTISKAKGQPREPQV | ESVTEQDSKDSTYSLS |
| | | | | YTLPPSRDELTKNQVSLTCLVKGFYPSDIAVE | STLTLSKADYEKHKVY |
| | | | | WESNGQPENNYKTTPPVLDSDGSFFLYSKLT | ACEVTHQGLSSPVTKS |
| | | | | VDKSRWQQGNVFSCSVMHEALHNHYTQKSL | FNRGEC |
| | | | | SLSPG | |
| CD117 | briqui- | 50 | 51 | QVQLVQSGAEVKKPGASVKVSCKASGYTFTS | DIVMTQSPDSLAVSLG |
| | limab | | | YNMHWVRQAPGQGLEWMGVIYSGNGDTSYN | ERATINCRASESVDIY |
| | | | | QKFKGRVTITADKSTSTAYMELSSLRSEDTAV | GNSFMHWYQQKPGQPP |
| | | | | YYCARERDTRFGNWGQGTLVTVSSASTKGPS | KLLIYLASNLESGVPD |
| | | | | VFPLAPSSKSTSGGTAALGCLVKDYFPEPVTV | RFSGSGSGTDFTLTIS |
| | | | | SWNSGALTSGVHTFPAVLQSSGLYSLSSVVTV | SLQAEDVAVYYCQQNN |
| | | | | PSSSLGTQTYICNVNHKPSNTKVDKKVEPKSC | EDPYTFGGGTKVEIKR |
| | | | | DKTHTCPPCPAPELLGGPSVFLFPPKPKDTLM | TVAAPSVFIFPPSDEQ |
| | | | | ISRTPEVTCVVVDVSHEDPEVKFNWYVDGVE | LKSGTASVVCLLNNFY |
| | | | | VHNAKTKPREEQYQSTYRVVSVLTVLHQDWL | PREAKVQWKVDNALQS |
| | | | | NGKEYKCKVSNKALPAPIEKTISKAKGQPREP | GNSQESVTEQDSKDST |
| | | | | QVYTLPPSRDELTKNQVSLTCLVKGFYPSDIA | YSLSSTLTLSKADYEK |
| | | | | VEWESNGQPENNYKTTPPVLDSDGSFFLYSK | HKVYACEVTHQGLSSP |
| | | | | LTVDKSRWQQGNVFSCSVMHEALHNHYTQK | VTKSFNRGEC |
| | | | | SLSLSPGK | |
| CD11a | efalizumab | 52 | 53 | EVQLVESGGGLVQPGGSLRLSCAASGYSFTG | DIQMTQSPSSLSASVG |
| | | | | HWMNWVRQAPGKGLEWVGMIHPSDSETRYN | DRVTITCRASKTISKY |
| | | | | QKFKDRFTISVDKSKNTLYLQMNSLRAEDTAV | LAWYQQKPGKAPKLLI |
| | | | | YYCARGIYFYGTTYFDYWGQGTLVTVSSASTK | YSGSTLQSGVPSRFSG |
| | | | | GPSVFPLAPSSKSTSGGTAALGCLVKDYFPEP | SGSGTDFTLTISSLQP |
| | | | | VTVSWNSGALTSGVHTFPAVLQSSGLYSLSSV | EDFATYYCQQHNEYPL |
| | | | | VTVPSSSLGTQTYICNVNHKPSNTKVDKKVEP | TFGQGTKVEIKRTVAA |
| | | | | KSCDKTHTCPPCPAPELLGGPSVFLFPPKPKD | PSVFIFPPSDEQLKSG |
| | | | | TLMISRTPEVTCVVVDVSHEDPEVKFNWYVD | TASVVCLLNNFYPREA |
| | | | | GVEVHNAKTKPREEQYNSTYRVVSVLTVLHQ | KVQWKVDNALQSGNSQ |
| | | | | DWLNGKEYKCKVSNKALPAPIEKTISKAKGQP | ESVTEQDSKDSTYSLS |
| | | | | REPQVYTLPPSREEMTKNQVSLTCLVKGFYPS | STLTLSKADYEKHKVY |
| | | | | DIAVEWESNGQPENNYKTTPPVLDSDGSFFLY | ACEVTHQGLSSPVTKS |
| | | | | SKLTVDKSRWQQGNVFSCSVMHEALHNHYT | FNRGEC |
| | | | | QKSLSLSPGK | |
| CD11b | MAB107- | 54 | 55 | QVQLVQSGAEVKKPGASVKVSCKPSGFNIKDI | DIVMTQSPDSLAVSLG |
| | CD11b | | | YMQWVRQAPGQRLEWIGRIDPADDKTKYDPK | ERATINCKSSQNLLYS |
| | binding | | | FQGRATITADTSASTAYLELSSLRSEDTAVYY | SNQKNYLAWYQQKPGQ |
| | fragment | | | CASEGHYGYDGYAMDYWGQGTTVTVSS | PPKLLIYWASTRESGV |
| | | | | | PDRFSGSGSGTDFTLT |
| | | | | | ISSLQAEDVAVYYCQQ |
| | | | | | YYSYPLTFGQGTKLEI |
| | | | | | K |
| CD137 | Acasunli- | 56 | 57 | EVQLVESGGGLVQPGRSLRLSCTASGFSLND | DIVMTQSPSSLSASVG |
| | mab- | | | YWMSWVRQAPGKGLEWVGYIDVGGSLYYAA | DRVTITCQASEDISSY |
| | CD137 | | | SVKGRFTISRDDSKSIAYLQMNSLKTEDTAVY | LAWYQQKPGKAPKRLI |
| | binding | | | YCARGGLTYGFDLWGQGTLVTVSSASTKGPSV | YGASDLASGVPSRFSA |
| | component | | | FPLAPSSKSTSGGTAALGCLVKDYFPEPVTVS | SGSGTDYTFTISSLQP |
| | | | | WNSGALTSGVHTFPAVLQSSGLYSLSSVVTVP | EDIATYYCHYYATISG |
| | | | | SSSLGTQTYICNVNHKPSNTKVDKRVEPKSCD | LGVAFGGGTKVEIKRT |
| | | | | KTHTCPPCPAPEFEGGPSVFLFPPKPKDTLMI | VAAPSVFIFPPSDEQL |
| | | | | SRTPEVTCVVVAVSHEDPEVKFNWYVDGVEV | KSGTASVVCLLNNFYP |
| | | | | HNAKTKPREEQYNSTYRVVSVLTVLHQDWLN | REAKVQWKVDNALQSG |
| | | | | GKEYKCKVSNKALPAPIEKTISKAKGQPREPQ | NSQESVTEQDSKDSTY |
| | | | | VYTLPPSREEMTKNQVSLTCLVKGFYPSDIAV | SLSSTLTLSKADYEKH |
| | | | | EWESNGQPENNYKTTPPVLDSDGSFFLYSRL | KVYACEVTHQGLSSPV |
| | | | | TVDKSRWQQGNVFSCSVMHEALHNHYTQKS | TKSFNRGEC |
| | | | | LSLSPG | |
| CD137 | ADG106 | 58 | 59 | EVQLVESGGGLVQPGGSLRLSCAASGFSLST | DIQLTQSPSSLSASVG |
| | | | | GGVGVGWIRQAPGKGLEWLALIDWADDKYYS | DRVTITCRASQSIGSY |
| | | | | PSLKSRLTISRDNSKNTLYLQLNSLRAEDTAV | LAWYQQKPGKAPKLLI |
| | | | | YYCARGGSDTVIGDWFAYWGQGTLVTVSSAST | YDASNLETGVPSRFSG |
| | | | | KGPSVFPLAPCSRSTSESTAALGCLVKDYFPE | SGSGTDFTLTISSLQP |
| | | | | PVTVSWNSGALTSGVHTFPAVLQSSGLYSLSS | EDFATYYCQQGYYLWT |
| | | | | VVTVPSSSLGTKTYTCNVDHKPSNTKVDKRVE | FGQGTKVEIKRTVAAP |
| | | | | SKYGPPCPPCPAPEFLGGPSVFLFPPKPKDTL | SVFIFPPSDEQLKSGT |
| | | | | MISRTPEVTCVVVDVSQEDPEVQFNWYVDGV | ASVVCLLNNFYPREAK |
| | | | | EVHNAKTKPREEQFNSTYRVVSVLTVLHQDW | VQWKVDNALQSGNSQE |
| | | | | LNGKEYKCKVSNKGLPSSIEKTISKAKGQPRE | SVTEQDSKDSTYSLSS |
| | | | | PQVYTLPPSQEEMTKNQVSLTQLVKGFYPSDI | TLTLSKADYEKHKVYA |
| | | | | AVEWESNGQPENNYKTTPPVLDSDGSFFLYS | CEVTHQGLSSPVTKSF |
| | | | | RLTVDKSRWQEGNVFSCSVMHEALHNHYTQ | NRGEC |
| | | | | KSLSLSLGK | |
| CD137 | cinreba- | 60 | 61 | EVQLVESGGGLVQPGGSLRLSCAASGFNIKDT | DIQMTQSPSSLSASVG |
| | fusp | | | YIHWVRQAPGKGLEWVARIYPTNGYTRYADS | DRVTITCRASQDVNTA |
| | | | | VKGRFTISADTSKNTAYLQMNSLRAEDTAVYY | VAWYQQKPGKAPKLLI |
| | | | | CSRWGGDGFYAMDYWGQGTLVTVSSASTKG | YSASFLYSGVPSRFSG |
| | | | | PSVFPLAPCSRSTSESTAALGCLVKDYFPEPV | SRSGTDFTLTISSLQP |
| | | | | TVSWNSGALTSGVHTFPAVLQSSGLYSLSSVV | EDFATYYCQQHYTTPP |
| | | | | TVPSSSLGTKTYTCNVDHKPSNTKVDKRVESK | TFGQGTKVEIKRTVAA |
| | | | | YGPPCPPCPAPEAAGGPSVFLFPPKPKDTLMI | PSVFIFPPSDEQLKSG |
| | | | | SRTPEVTCVVVDVSQEDPEVQFNWYVDGVEV | TASVVCLLNNFYPREA |
| | | | | HNAKTKPREEQFNSTYRVVSVLTVLHQDWLN | KVQWKVDNALQSGNSQ |
| | | | | GKEYKCKVSNKGLPSSIEKTISKAKGQPREPQ | ESVTEQDSKDSTYSLS |
| | | | | VYTLPPSQEEMTKNQVSLTCLVKGFYPSDIAV | STLTLSKADYEKHKVY |
| | | | | EWESNGQPENNYKTTPPVLDSDGSFFLYSRL | ACEVTHQGLSSPVTKS |
| | | | | TVDKSRWQEGNVFSCSVMHEALHNHYTQKSL | FNRGEC |
| | | | | SLSLGKGGGGSGGGGSGGGGSQDSTSDLIP | |
| | | | | APPLSKVPLQQNFQDNQFHGKWYVVGQAGNI | |
| | | | | RLREDKDPIKMMATIYELKEDKSYDVTMVKFD | |
| | | | | DKKCMYDIWTFVPGSQPGEFTLGKIKSFPGHT | |
| | | | | SSLVRVVSTNYNQHAMVFFKFVFQNREEFYIT | |
| | | | | LYGRTKELTSELKENFIRFSKSLGLPENHIVF | |
| | | | | PVPIDQCIDG | |
| CD137 | ensoma- | 62 | 63 | QVQLVQSGAEVKKPGASVKVSCKASGYTFTD | DIVMTQTPLSLSVTPG |
| | fusp | | | YIMHWVRQAPGQGLEWMGYINPYNDGSKYT | QPASISCKSSQSLETS |
| | | | | EKFQGRVTMTSDTSISTAYMELSRLRSDDTAV | TGTTYLNWYLQKPGQS |
| | | | | YYCARGTYYYGPQLFDYWGQGTTVTVSSAST | PQLLIYRVSKRFSGVP |
| | | | | KGPSVFPLAPSSKSTSGGTAALGCLVKDYFPE | DRFSGSGSGTDFTLKI |
| | | | | PVTVSWNSGALTSGVHTFPAVLQSSGLYSLSS | SRVEAEDVGVYYCLQL |
| | | | | VVTVPSSSLGTQTYICNVNHKPSNTKVDKKVE | LEDPYTFGQGTKLEIK |
| | | | | PKSCDKTHTCPPCPAPEAAGGPSVFLFPPKPK | RTVAAPSVFIFPPSDE |
| | | | | DTLMISRTPEVTCVVVDVSHEDPEVKFNWYVD | QLKSGTASVVCLLNNF |
| | | | | GVEVHNAKTKPREEQYNSTYRVVSVLTVLHQ | YPREAKVQWKVDNALQ |
| | | | | DWLNGKEYKCKVSNKALGAPIEKTISKAKGQP | SGNSQESVTEQDSKDS |
| | | | | REPQVCTLPPSRDELTKNQVSLSCAVKGFYPS | TYSLSSTLTLSKADYE |
| | | | | DIAVEWESNGQPENNYKTTPPVLDSDGSFFLV | KHKVYACEVTHQGLSS |
| | | | | SKLTVDKSRWQQGNVFSCSVMHEALHNHYT | PVTKSFNRGEC |
| | | | | QKSLSLSPGK | |
| CD137 | LVGN6051- | 64 | 65 | QVQLVQSGAEVKKPGASVKVSCKASGYTFAG | DIQMTQSPSSLSASVG |
| | CD137 | | | FEMHWVRQAPGQGLEWMGAIDPKTGGTDYN | DRVTITCRASQDIRSN |
| | binding | | | QKFKDRVTMTRDTSISTAYMELSRLRSDDTAV | LNWYQQKPGGAVKLLI |
| | fragment | | | YYCARDLGYFDVWGQGTLVTVSS | YYTSRLHSGVPSRFSG |
| | | | | | SGSGTDYTLTISSLQP |
| | | | | | EDFATYFCQQSEKLPR |
| | | | | | TFGGGTKVEIRR |
| CD137 | PRS-343 | 66 | 67 | EVQLVESGGGLVQPGGSLRLSCAASGFNIKDT | DIQMTQSPSSLSASVG |
| | component | | | YIHWVRQAPGKGLEWVARIYPTNGYTRYADS | DRVTITCRASQDVNTA |
| | J10 | | | VKGRFTISADTSKNTAYLQMNSLRAEDTAVYY | VAWYQQKPGKAPKLLI |
| | anticalin | | | CSRWGGDGFYAMDYWGQGTLVTVSS | YSASFLYSGVPSRFSG |
| | protein | | | | SRSGTDFTLTISSLQP |
| | | | | | EDFATYYCQQHYTTPP |
| | | | | | TFGQGTKVEIK |
| CD137 | tecagin- | 68 | 69 | EVQLVESGGGLVQPGRSLRLSCTASGFSLND | DIVMTQSPSSLSASVG |
| | limab | | | YWMSWVRQAPGKGLEWVGYIDVGGSLYYAA | DRVTITCQASEDISSY |
| | | | | SVKGRFTISRDDSKSIAYLQMNSLKTEDTAVY | LAWYQQKPGKAPKRLI |
| | | | | YCARGGLTYGFDLWGQGTLVTVSSASTKGPSV | YGASDLASGVPSRFSA |
| | | | | FPLAPSSKSTSGGTAALGCLVKDYFPEPVTVS | SGSGTDYTFTISSLQP |
| | | | | WNSGALTSGVHTFPAVLQSSGLYSLSSVVTVP | EDIATYYCHYYATISG |
| | | | | SSSLGTQTYICNVNHKPSNTKVDKRVEPKSCD | LGVAFGGGTKVEIKRT |
| | | | | KTHTCPPCPAPEFEGGPSVFLFPPKPKDTLMI | VAAPSVFIFPPSDEQL |
| | | | | SRTPEVTCVVVAVSHEDPEVKFNWYVDGVEV | KSGTASVVCLLNNFYP |
| | | | | HNAKTKPREEQYNSTYRVVSVLTVLHQDWLN | REAKVQWKVDNALQSG |
| | | | | GKEYKCKVSNKALPAPIEKTISKAKGQPREPQ | NSQESVTEQDSKDSTY |
| | | | | VYTLPPSREEMTKNQVSLTCLVKGFYPSDIAV | SLSSTLTLSKADYEKH |
| | | | | EWESNGQPENNYKTTPPVLDSDGSFFLYSRL | KVYACEVTHQGLSSPV |
| | | | | TVDKSRWQQGNVFSCSVMHEALHNHYTQKS | TKSFNRGEC |
| | | | | LSLSPG | |
| CD137 | urelumab | 70 | 71 | QVQLQQWGAGLLKPSETLSLTCAVYGGSFSG | EIVLTQSPATLSLSPG |
| | | | | YYWSWIRQSPEKGLEWIGEINHGGYVTYNPSL | ERATLSCRASQSVSSY |
| | | | | ESRVTISVDTSKNQFSLKLSSVTAADTAVYYC | LAWYQQKPGQAPRLLI |
| | | | | ARDYGPGNYDWYFDLWGRGTLVTVSSASTKG | YDASNRATGIPARFSG |
| | | | | PSVFPLAPCSRSTSESTAALGCLVKDYFPEPV | SGSGTDFTLTISSLEP |
| | | | | TVSWNSGALTSGVHTFPAVLQSSGLYSLSSVV | EDFAVYYCQQRSNWPP |
| | | | | TVPSSSLGTKTYTCNVDHKPSNTKVDKRVESK | ALTFCGGTKVEIKRTV |
| | | | | YGPPCPPCPAPEFLGGPSVFLFPPKPKDTLMI | AAPSVFIFPPSDEQLK |
| | | | | SRTPEVTCVVVDVSQEDPEVQFNWYVDGVEV | SGTASVVCLLNNFYPR |
| | | | | HNAKTKPREEQFNSTYRVVSVLTVLHQDWLN | EAKVQWKVDNALQSGN |
| | | | | GKEYKCKVSNKGLPSSIEKTISKAKGQPREPQ | SQESVTEQDSKDSTYS |
| | | | | VYTLPPSQEEMTKNQVSLTCLVKGFYPSDIAV | LSSTLTLSKADYEKHK |
| | | | | EWESNGQPENNYKTTPPVLDSDGSFFLYSRL | VYACEVTHQGLSSPVT |
| | | | | TVDKSRWQEGNVFSCSVMHEALHNHYTQKSL | KSFNRGEC |
| | | | | SLSLGK | |
| CD137 | utomilumab | 72 | 73 | EVQLVQSGAEVKKPGESLRISCKGSGYSFSTY | SYELTQPPSVSVSPGQ |
| | | | | WISWVRQMPGKGLEWMGKIYPGDSYTNYSP | TASITCSGDNIGDQYA |
| | | | | SFQGQVTISADKSISTAYLQWSSLKASDTAMY | HWYQQKPGQSPVLVIY |
| | | | | YCARGYGIFDYWGQGTLVTVSSASTKGPSVF | QDKNRPSGIPERFSGS |
| | | | | PLAPCSRSTSESTAALGCLVKDYFPEPVTVSW | NSGNTATLTISGTQAM |
| | | | | NSGALTSGVHTFPAVLQSSGLYSLSSVVTVPS | DEADYYCATYTGFGSL |
| | | | | SNFGTQTYTCNVDHKPSNTKVDKTVERKCCV | AVFGGGTKLTVLGQPK |
| | | | | ECPPCPAPPVAGPSVFLFPPKPKDTLMISRTP | AAPSVTLFPPSSEELQ |
| | | | | EVTCVVVDVSHEDPEVQFNWYVDGVEVHNAK | ANKATLVCLISDFYPG |
| | | | | TKPREEQFNSTFRVVSVLTVVHQDWLNGKEY | AVTVAWKADSSPVKAG |
| | | | | KCKVSNKGLPAPIEKTISKTKGQPREPQVYTL | VETTTPSKQSNNKYAA |
| | | | | PPSREEMTKNQVSLTCLVKGFYPSDIAVEWES | SSYLSLTPEQWKSHRS |
| | | | | NGQPENNYKTTPPMLDSDGSFFLYSKLTVDKS | YSCQVTHEGSTVEKTV |
| | | | | RWQQGNVFSCSVMHEALHNHYTQKSLSLSP | APTECS |
| | | | | GK | |
| CD2 | Alefacept- | 74 | 75 | CFSQQIYGVVYGNVTFHVPSNVPLKEVLWKK | MVAGSDAGRALGVLSV |
| | fusion of | | | QKDKVAELENSEFRAFSSFKNRVYLDTVSGSL | VCLLHCFGFISCFSQQ |
| | LFA-3 (CD2 | | | TIYNLTSSDEDEYEMESPNITDTMKFFLYVLE | IYGVVYGNVTFHVPSN |
| | ligand) | | | SLPSPTLTCALTNGSIEVQCMIPEHYNSHRGL | VPLKEVLWKKQKDKVA |
| | and | | | IMYSWDCPMEQCKRNSTSIYFKMENDLPQKIQ | ELENSEFRAFSSFKNR |
| | immunoglo- | | | TCLSNPLFNTTSSIILTTCIPSSGHSRHRYAL | VYLDTVSGSLTIYNLT |
| | bulin G1 | | | IPIPLAVITTCIVLYMNGILKCDRKPDRTNSN | SSDEDEYEMESPNITD |
| | | | | RVEPKSCDKTHTCPPCPAPELLGGPSVFLFPP | TMKFFLYVLESLPSPT |
| | | | | KPKDTLMISRTPEVTCVVVDVSHEDPQVKFNW | LTCALTNGSIEVQCMI |
| | | | | YVDGVQVHNAKTKPREQQYNSTYRVVSVLTVL | PEHYNSHRGLIMYSWD |
| | | | | HQNWLDGKEYKCKVSNKALPAPIEKTISKAKG | CPMEQCKRNSTSIYFK |
| | | | | QPREPQVYTLPPSREEMTKNQVSLTCLVKGFY | MENDLPQKIQCTLSNP |
| | | | | PSDIAVEWESNGQPENNYKTTPPVLDSDGSFF | LFNTTSSIILTTCIPS |
| | | | | LYSKLTVDKSRWQQGNVFSCSVMHEALHNHYT | SGHSRHRYALIPIPLA |
| | | | | QKSLSLSPGK | VITTCIVLYMNGILKC |
| | | | | | DRKPDRTNSN |
| CD2 | BTI-322- | 76 | 77 | EVQLQQSGPELQRPGASVKLSCKASGYIFTEY | DVVLTQTPPTLLATIG |
| | CD2 | | | YMYWVKQRPKQGLELVGRIDPEDGSIDYVEK | QSVSISCRSSQSLLHS |
| | binding | | | FKKKATLTADTSSNTAYMQLSSLTSEDTATYF | SGNTYLNWLLQRTGQS |
| | fragment | | | CARGKFNYRFAYWGQGTLVTVSS | PQPLIYLVSKLESGVP |
| | | | | | NRFSGSGSGTDFTLKI |
| | | | | | SGVEAEDLGVYYCMQF |
| | | | | | THYPYTFGAGTKLELK |
| CD2 | HuMCD2- | 78 | | TNALETWGALGQDIELNIPSFQMSDDIDDIKW | |
| | CD2 | | | EKTSDKKKIAQFRKEKETFKEKDGGSGGLGAS | |
| | binding | | | WHRPDKFCLGYQKRPLPGGSGGTYELDKNG | |
| | fragment | | | DLDIKHLKTDDQDIYKVSIYDTKGKNVLEKIF | |
| | | | | DLKIQE | |
| CD2 | Sipli- | 79 | 80 | QVQLVQSGAEVKKPGASVKVSCKASGYTFTG | DVVMTQSPPSLLVTLG |
| | zumab- | | | YYMHWVRQAPGQGLEWMGRINPNSGGTNYA | QPASISCRSSQSLLHS |
| | CD2 | | | QKFQGRVTMTRDTSISTAYMELSRLRSDDTAV | SGNTYLNWLLQRPGQS |
| | binding | | | YYCARGRTEYIVVAEGFDYWGQGTLVTVSS | PQPLIYLVSKLESGVP |
| | fragment | | | | DRFSGSGSGTDFTLKI |
| | | | | | SGVEAEDVGVYYCMQF |
| | | | | | THYPYTFGQGTKLEIK |
| CD2 | T11.2-CD2 | 81 | 82 | QVQLQQPGAELVRPGASVKLSCKASGYTFTT | DIVMTQSPASLAVSLG |
| | binding | | | FWMNWVKQRPGQGLEWIGMIDPSDSEAHYN | QRATISYRASKSVSTS |
| | fragment | | | QMFKDKATLTVDKSSSTAYMQLSSLTSEDSAV | GYSYMHWNQQKPGQPP |
| | | | | YYCARGRGYDDGDAMDYWGQGTSVTVSS | RLLIYLVSNLESGVPA |
| | | | | | RFSGSGSGTDFTLNIH |
| | | | | | PVEEEDAATYYCMQFT |
| | | | | | HYPYTFGGGTKLEIK |
| CD2 | TS2/18-CD2 | 83 | 84 | EVQLEESGGGLVMPGGSLKLSCAASGFAFSS | DIVMTQSPATLSVTPG |
| | binding | | | YDMSWVRQTPEKRLEWVAYISGGGFTYYPDT | DRVFLSCRASQSISDF |
| | fragment | | | VKGRFTLSRDNAKNTLYLQMSSLKSEDTAMY | LHWYQQKSHESPRLLI |
| | | | | YCARQGANWELVYWGQGTTLTVSS | KYASQSISGIPSRFSG |
| | | | | | SGSGSDFTLSINSVEP |
| | | | | | EDVGVYLCQNGHNFPP |
| | | | | | TFGGGTKLEIK |
| CD2 | OKT11-CD2 | 85 | 86 | QVQLQQPGAELVRPGTSVKLSCKASGYTFTS | DIVMTQAAPSVPVTPG |
| | binding | | | YWMHWIKQRPEQGLEWIGRIDPYDSETHYNE | ESVSISCRSSKTLLHS |
| | fragment | | | KFKDKAILSVDKSSSTAYIQLSSLTSDDSAVY | NGNTYLYWFLQRPGQS |
| | | | | YCSRRDAKYDGYALDYWGQGTTLTVSS | PQVLIYRMSNLASGVP |
| | | | | | NRFSGSGSETTFTLRI |
| | | | | | SRVEAEDVGIYYCMQH |
| | | | | | LEYPYTFGGGTKLEIK |
| CD200 | samali- | 87 | 88 | QVQLQQSGSELKKPGASVKISCKASGYSFTDY | DIQMTQSPSSLSASIG |
| | zumab | | | IILWVRQNPGKGLEWIGHIDPYYGSSNYNLKF | DRVTITCKASQDINSY |
| | | | | KGRVTITADQSTTTAYMELSSLRSEDTAVYYC | LSWFQQKPGKAPKLLI |
| | | | | GRSKRDYFDYWGQGTTLTVSSASTKGPSVFPL | YRANRLVDGVPSRFSG |
| | | | | APCSRSTSESTAALGCLVKDYFPEPVTVSWN | SGSGTDYTLTISSLQP |
| | | | | SGALTSGVHTFPAVLQSSGLYSLSSVVTVPSS | EDFAVYYCLQYDEFPY |
| | | | | NFGTQTYTCNVDHKPSNTKVDKTVERKCCVE | TFGGGTKLEIKRTVAA |
| | | | | CPPCPAPPVAGPSVFLFPPKPKDTLMISRTPE | PSVFIFPPSDEQLKSG |
| | | | | VTCVVVDVSQEDPEVQFNWYVDGVEVHNAKT | TASVVCLLNNFYPREA |
| | | | | KPREEQFNSTYRVVSVLTVLHQDWLNGKEYK | KVQWKVDNALQSGNSQ |
| | | | | CKVSNKGLPSSIEKTISKAKGQPREPQVYTLP | ESVTEQDSKDSTYSLS |
| | | | | PSQEEMTKNQVSLTCLVKGFYPSDIAVEWESN | STLTLSKADYEKHKVY |
| | | | | GQPENNYKTTPPVLDSDGSFFLYSRLTVDKSR | ACEVTHQGLSSPVTKS |
| | | | | WQEGNVFSCSVMHEALHNHYTQKSLSLSLG | FNRGEC |
| CD25 | basili- | 89 | 90 | EVQLQQSGTVLARPGASVKMSCKASGYSFTR | QIVLTQSPAIMSASPG |
| | ximab | | | YWMHWIKQRPGQGLEWIGAIYPGNSDTSYNQ | EKVTMTCSASSSISYM |
| | | | | KFEGKAKLTAVTSASTAYMELSSLTHEDSAVY | QWYQQKPGTSPKRWIY |
| | | | | YCSRDYGYYFDFWGQGTTLTVSSASTKGPSV | DTSKLASGVPARFSGS |
| | | | | FPLAPSSKSTSGGTAALGCLVKDYFPEPVTVS | GSGTSYSLTISSMEAE |
| | | | | WNSGALTSGVHTFPAVLQSSGLYSLSSVVTVP | DAATYYCHQRSSYTFG |
| | | | | SSSLGTQTYICNVNHKPSNTKVDKRVEPKSCD | GGTKLEIKRTVAAPSV |
| | | | | KTHTCPPCPAPELLGGPSVFLFPPKPKDTLMI | FIFPPSDEQLKSGTAS |
| | | | | SRTPEVTCVVVDVSHEDPEVKFNWYVDGVEVH | VVCLLNNFYPREAKVQ |
| | | | | NAKTKPREEQYNSTYRVVSVLTVLHQDWLNG | WKVDNALQSGNSQESV |
| | | | | KEYKCKVSNKALPAPIEKTISKAKGQPREPQV | TEQDSKDSTYSLSSTL |
| | | | | YTLPPSREEMTKNQVSLTCLVKGFYPSDIAVE | TLSKADYEKHKVYACE |
| | | | | WESNGQPENNYKTTPPVLDSDGSFFLYSKLT | VTHQGLSSPVTKSFNR |
| | | | | VDKSRWQQGNVFSCSVMHEALHNHYTQKSL | GEC |
| | | | | SLSPGK | |
| CD25 | camidan- | 91 | 92 | QVQLVQSGAEVKKPGSSVKVSCKASGGTFSR | EIVLTQSPGTLSLSPG |
| | lumab | | | YIINWVRQAPGQGLEWMGRIIPILGVENYAQKF | ERATLSCRASQSVSSY |
| | | | | QGRVTITADKSTSTAYMELSSLRSEDTAVYYC | LAWYQQKPGQAPRLLI |
| | | | | ARKDWFDYWGQGTLVTVSSASTKGPSVFPLA | YGASSRATGIPDRFSG |
| | | | | PSSKSTSGGTAALGCLVKDYFPEPVTVSWNS | SGSGTDFTLTISRLEP |
| | | | | GALTSGVHTFPAVLQSSGLYSLSSVVTVPSSS | EDFAVYYCQQYGSSPL |
| | | | | LGTQTYICNVNHKPSNTKVDKRVEPKSCDKTH | TFGGGTKVEIKRTVAA |
| | | | | TCPPCPAPELLGGPSVFLFPPKPKDTLMISRTP | PSVFIFPPSDEQLKSG |
| | | | | EVTCVVVDVSHEDPEVKFNWYVDGVEVHNAK | TASVVCLLNNFYPREA |
| | | | | TKPREEQYNSTYRVVSVLTVLHQDWLNGKEY | KVQWKVDNALQSGNSQ |
| | | | | KCKVSNKALPAPIEKTISKAKGQPREPQVYTLP | ESVTEQDSKDSTYSLS |
| | | | | PSREEMTKNQVSLTCLVKGFYPSDIAVEWESN | STLTLSKADYEKHKVY |
| | | | | GQPENNYKTTPPVLDSDGSFFLYSKLTVDKSR | ACEVTHQGLSSPVTKS |
| | | | | WQQGNVFSCSVMHEALHNHYTQKSLSLSPG | FNRGEC |
| | | | | K | |
| CD25 | daclizumab | 93 | 94 | QVQLVQSGAEVKKPGSSVKVSCKASGYTFTS | DIQMTQSPSTLSASVG |
| | | | | YRMHWVRQAPGQGLEWIGYINPSTGYTEYNQ | DRVTITCSASSSISYM |
| | | | | KFKDKATITADESTNTAYMELSSLRSEDTAVYY | HWYQQKPGKAPKLLIY |
| | | | | CARGGGVFDYWGQGTLVTVSSASTKGPSVFP | TTSNLASGVPARFSGS |
| | | | | LAPSSKSTSGGTAALGCLVKDYFPEPVTVSW | GSGTEFTLTISSLQPD |
| | | | | NSGALTSGVHTFPAVLQSSGLYSLSSVVTVPS | DFATYYCHQRSTYPLT |
| | | | | SSLGTQTYICNVNHKPSNTKVDKKVEPKSCDK | FGQGTKVEVKRTVAAP |
| | | | | THTCPPCPAPELLGGPSVFLFPPKPKDTLMIS | SVFIFPPSDEQLKSGT |
| | | | | RTPEVTCVVVDVSHEDPEVKFNWYVDGVEVH | ASVVCLLNNFYPREAK |
| | | | | NAKTKPREEQYNSTYRVVSVLTVLHQDWLNG | VQWKVDNALQSGNSQE |
| | | | | KEYKCKVSNKALPAPIEKTISKAKGQPREPQV | SVTEQDSKDSTYSLSS |
| | | | | YTLPPSRDELTKNQVSLTCLVKGFYPSDIAVE | TLTLSKADYEKHKVYA |
| | | | | WESNGQPENNYKTTPPVLDSDGSFFLYSKLT | CEVTHQGLSSPVTKSF |
| | | | | VDKSRWQQGNVFSCSVMHEALHNHYTQKSL | NRGEC |
| | | | | SLSPGK | |
| B7H3 | Enobli- | 95 | 96 | EVQLVESGGGLVQPGGSLRLSCAASGFTFSS | DIQLTQSPSFLSASVG |
| (CD276) | tuzumab | | | FGMHWVRQAPGKGLEWVAYISSDSSAIYYAD | DRVTITCKASQNVDTN |
| | | | | TVKGRFTISRDNAKNSLYLQMNSLRDEDTAVY | VAWYQQKPGKAPKALI |
| | | | | YCGRGRENIYYGSRLDYWGQGTTVTVSSAST | YSASYRYSGVPSRFSG |
| | | | | KGPSVFPLAPSSKSTSGGTAALGCLVKDYFPE | SGSGTDFTLTISSLQP |
| | | | | PVTVSWNSGALTSGVHTFPAVLQSSGLYSLSS | EDFATYYCQQYNNYPF |
| | | | | VVTVPSSSLGTQTYICNVNHKPSNTKVDKRVE | TFGQGTKLEIKRTVAA |
| | | | | PKSCDKTHTCPPCPAPELVGGPSVFLLPPKPK | PSVFIFPPSDEQLKSG |
| | | | | DTLMISRTPEVTCVVVDVSHEDPEVKFNWYVD | TASVVCLLNNFYPREA |
| | | | | GVEVHNAKTKPPEEQYNSTLRVVSVLTVLHQD | KVQWKVDNALQSGNSQ |
| | | | | WLNGKEYKCKVSNKALPAPIEKTISKAKGQPR | ESVTEQDSKDSTYSLS |
| | | | | EPQVYTLPPSREEMTKNQVSLTCLVKGFYPSD | STLTLSKADYEKHKVY |
| | | | | IAVEWESNGQPENNYKTTPLVLDSDGSFFLYS | ACEVTHQGLSSPVTKS |
| | | | | KLTVDKSRWQQGNVFSCSVMHEALHNHYTQ | FNRGEC |
| | | | | KSLSLSPGK | |
| B7H3 | Omburtamab | 97 | 98 | QVQLQQSGAELVKPGASVKLSCKASGYTFTN | DIVMTQSPATLSVTPG |
| | | | | YDINWVRQRPEQGLEWIGWIFPGDGSTQYNE | DRVSLSCRASQSISDY |
| | | | | KFKGKATLTTDTSSSTAYMQLSRLTSEDSAVY | LHWYQQKSHESPRLLI |
| | | | | FCARQTTATWFAYWGQGTLVTVSAAKTTPPS | KYASQSISGIPSRFSG |
| | | | | VYPLAPGSAAQTNSMVTLGCLVKGYFPEPVTV | SGSGSDFTLSINSVEP |
| | | | | TWNSGSLSSGVHTFPAVLQSDLYTLSSSVTVP | EDVGVYYCQNGHSFPL |
| | | | | SSTWPSETVTCNVAHPASSTKVDKKIVPRDCG | TFGAGTKLELKRADAA |
| | | | | CKPCICTVPEVSSVFIFPPKPKDVLTITLTPK | PTVSIFPPSSEQLTSG |
| | | | | VTCVVVDISKDDPEVQFSWFVDDVEVHTAQTQ | GASVVCFLNNFYPKDI |
| | | | | PREEQFNSTFRSVSELPIMHQDWLNGKEFKCR | NVKWKIDGSERQNGVL |
| | | | | VNSAAFPAPIEKTISKTKGRPKAPQVYTIPPP | NSWTDQDSKDSTYSMS |
| | | | | KEQMAKDKVSLTCMITDFFPEDITVEWQWNGQ | STLTLTKDEYERHNSY |
| | | | | PAENYKNTQPIMDTDGSYFVYSKLNVQKSNW | TCEATHKTSTSPIVKS |
| | | | | EAGNTFTCSVLHEGLHNHHTEKSLSHSPGK | FNRNEC |
| B7H3 | Obrinda- | 99 | 100 | DIQLTQSPSFLSASVGDRVTITCKASQNVDTN | QAVVTQEPSLTVSPGG |
| | tamab | | | VAWYQQKPGKAPKALIYSASYRYSGVPSRFS | TVTLTCRSSTGAVTTS |
| | | | | GSGSGTDFTLTISSLQPEDFATYYCQQYNNYP | NYANWVQQKPGQAPRG |
| | | | | FTFGQGTKLEIKGGGSGGGGEVQLVESGGGL | LIGGTNKRAPWTPARF |
| | | | | VQPGGSLRLSCAASGFTFSTYAMNWVRQAP | SGSLLGGKAALTITGA |
| | | | | GKGLEWVGRIRSKYNNYATYYADSVKDRFTIS | QAEDEADYYCALWYSN |
| | | | | RDDSKNSLYLQMNSLKTEDTAVYYCVRHGNF | LWVFGGGTKLTVLGGG |
| | | | | GNSYVSWFAYWGQGTLVTVSSGGGGGGEVA | GSGGGGEVQLVESGGG |
| | | | | ALEKEVAALEKEVAALEKEVAALEKGGGDKTH | LVQPGGSLRLSCAASG |
| | | | | TCPPCPAPEAAGGPSVFLFPPKPKDTLMISRT | FTFSSFGMHWVRQAPG |
| | | | | PEVTCVVVDVSHEDPEVKFNWYVDGVEVHNA | KGLEWVAYISSDSSAI |
| | | | | KTKPREEQYNSTYRVVSVLTVLHQDWLNGKE | YYADTVKGRFTISRDN |
| | | | | YKCKVSNKALPAPIEKTISKAKGQPREPQVYT | AKNSLYLQMNSLRDED |
| | | | | LPPSREEMTKNQVSLWCLVKGFYPSDIAVEWE | TAVYYCGRGRENIYYG |
| | | | | SNGQPENNYKTTPPVLDSDGSFFLYSKLTVDK | SRLDYWGQGTTVTVSS |
| | | | | SRWQQGNVFSCSVMHEALHNHYTQKSLSLSP | GGCGGGKVAALKEKVA |
| | | | | Gk | ALKEKVAALKEKVAAL |
| | | | | | KE |
| B7H3 | Ifinatamab | 101 | 102 | QVQLVQSGAEVKKPGSSVKVSCKASGYTFTN | EIVLTQSPATLSLSPG |
| | | | | YVMHWVRQAPGQGLEWMGYINPYNDDVKYN | ERATLSCRASSRLIYM |
| | | | | EKFKGRVTITADESTSTAYMELSSLRSEDTAV | HWYQQKPGQAPRPLIY |
| | | | | YYCARWGYYGSPLYYFDYWGQGTLVTVSSA | ATSNLASGIPARFSGS |
| | | | | STKGPSVFPLAPSSKSTSGGTAALGCLVKDYF | GSGTDFTLTISSLEPE |
| | | | | PEPVTVSWNSGALTSGVHTFPAVLQSSGLYSL | DFAVYYCQQWNSNPPT |
| | | | | SSVVTVPSSSLGTQTYICNVNHKPSNTKVDKR | FGQGTKVEIKRTVAAP |
| | | | | VEPKSCDKTHTCPPCPAPELLGGPSVFLFPPK | SVFIFPPSDEQLKSGT |
| | | | | PKDTLMISRTPEVTCVVVDVSHEDPEVKFNWY | ASVVCLLNNFYPREAK |
| | | | | VDGVEVHNAKTKPREEQYNSTYRVVSVLTVL | VQWKVDNALQSGNSQE |
| | | | | HQDWLNGKEYKCKVSNKALPAPIEKTISKAKG | SVTEQDSKDSTYSLSS |
| | | | | QPREPQVYTLPPSREEMTKNQVSLTCLVKGF | TLTLSKADYEKHKVYA |
| | | | | YPSDIAVEWESNGQPENNYKTTPPVLDSDGS | CEVTHQGLSSPVTKSF |
| | | | | FFLYSKLTVDKSRWQQGNVFSCSVMHEALHN | NRGEC |
| | | | | HYTQKSLSLSPGK | |
| B7H3 | mirzotamab | 103 | 104 | EVQLQESGPGLVKPSETLSLTCAVTGYSITSG | DIQMTQSPSSLSASVG |
| | | | | YSWHWIRQFPGNGLEWMGYIHSSGSTNYNP | DRVTITCKASQNVGFN |
| | | | | SLKSRISISRDTSKNQFFLKLSSVTAADTAVY | VAWYQQKPGKSPKALI |
| | | | | YCAGYDDYFEYWGQGTTVTVSSASTKGPSVFP | YSASYRYSGVPSRFSG |
| | | | | LAPSSKSTSGGTAALGCLVKDYFPEPVTVSW | SGSGTDFTLTISSLQP |
| | | | | NSGALTSGVHTFPAVLQSSGLYSLSSVVTVPS | EDFAEYFCQQYNWYPF |
| | | | | SSLGTQTYICNVNHKPSNTKVDKKVEPKSCDK | TFGQGTKLEIKRTVAA |
| | | | | THTCPPCPAPEAAGGPSVFLFPPKPKDTLMIS | PSVFIFPPSDEQLKSG |
| | | | | RTPEVTCVVVDVSHEDPEVKFNWYVDGVEVH | TASVVCLLNNFYPREA |
| | | | | NAKTKPREEQYNSTYRVVSVLTVLHQDWLNG | KVQWKVDNALQSGNSQ |
| | | | | KEYKCKVSNKALPAPIEKTISKAKGQPREPQV | ESVTEQDSKDSTYSLS |
| | | | | YTLPPSREEMTKNQVSLTCLVKGFYPSDIAVE | STLTLSKADYEKHKVY |
| | | | | WESNGQPENNYKTTPPVLDSDGSFFLYSKLT | ACEVTHQGLSSPVTKS |
| | | | | VDKSRWQQGNVFSCSVMHEALHNHYTQKSL | FNRGEC |
| | | | | SLSPGK | |
| B7H3 | TRL4542 | 105 | 106 | QVQLVESGGDLVQPGESLRLSCAASGFIFSDA | DIEMTQSPDSLAYSLG |
| | | | | WMVWVRQAPGKGLEWVGRIKTNGDGGTTDL | ERATINCKSSHNLLYK |
| | | | | TEPVKGRFTISRDDSKNMVYLQMNNLRTEDTA | SNNKNYLAWSQQKPGQ |
| | | | | IYYCTTAPGFWGQGTLVTVSS | PPRLLIYWASTRDSGV |
| | | | | | PDRFSGSGSGTDFTLT |
| | | | | | ISSLQAEDVAVYYCHQ |
| | | | | | YYGTKWTFGQGTRVEI |
| | | | | | KR |
| B7H3 | MGC018 | 107 | 108 | EVQLVESGGGLVKPGGSLRLSCAASGFTFSS | DIQMTQSPSSLSASVG |
| | | | | YGMSWVRQAPGKGLEWVATINSGGSNTYYP | DRVTITCRASESIYSY |
| | | | | DSLKGRFTISRDNAKNSLYLQMNSLRAEDTAV | LAWYQQKPGKAPKLLV |
| | | | | YYCARHDGGAMDYWGQGTTVTVSSASTKGP | YNTKTLPEGVPSRFSG |
| | | | | SVFPLAPSSKSTSGGTAALGCLVKDYFPEPVT | SGSGTDFTLTISSLQP |
| | | | | VSWNSGALTSGVHTFPAVLQSSGLYSLSSVVT | EDFATYYCQHHYGTPP |
| | | | | VPSSSLGTQTYICNVNHKPSNTKVDKRVEPKS | WTFGQGTRLEIKRTVA |
| | | | | CDKTHTCPPCPAPELLGGPSVFLFPPKPKDTL | APSVFIFPPSDEQLKS |
| | | | | MISRTPEVTCVVVDVSHEDPEVKFNWYVDGV | GTASVVCLLNNFYPRE |
| | | | | EVHNAKTKPREEQYNSTYRVVSVLTVLHQDW | AKVQWKVDNALQSGNS |
| | | | | LNGKEYKCKVSNKALPAPIEKTISKAKGQPRE | QESVTEQDSKDSTYSL |
| | | | | PQVYTLPPSREEMTKNQVSLTCLVKGFYPSDI | SSTLTLSKADYEKHKV |
| | | | | AVEWESNGQPENNYKTTPPVLDSDGSFFLYSK | YACEVTHQGLSSPVTK |
| | | | | LTVDKSRWQQGNVFSCSVMHEALHNHYTQK | SFNRGEC |
| | | | | SLSLSPGK | |
| B7H3 | DS-7300a | 109 | 110 | QVQLVQSGAEVKKPGSSVKVSCKASGYTFTN | EIVLTQSPATLSLSPG |
| | | | | YVMHWVRQAPGQGLEWMGYINPYNDDVKYN | ERATLSCRASSRLIYM |
| | | | | EKFKGRVTITADESTSTAYMELSSLRSEDTAV | HWYQQKPGQAPRPLIY |
| | | | | YYCARWGYYGSPLYYFDYWGQGTLVTVSSA | ATSNLASGIPARFSGS |
| | | | | STKGPSVFPLAPSSKSTSGGTAALGCLVKDYF | GSGTDFTLTISSLEPE |
| | | | | PEPVTVSWNSGALTSGVHTFPAVLQSSGLYSL | DFAVYYCQQWNSNPPT |
| | | | | SSVVTVPSSSLGTQTYICNVNHKPSNTKVDKR | FGQGTKVEIKRTVAAP |
| | | | | VEPKSCDKTHTCPPCPAPELLGGPSVFLFPPK | SVFIFPPSDEQLKSGT |
| | | | | PKDTLMISRTPEVTCVVVDVSHEDPEVKFNWY | ASVVCLLNNFYPREAK |
| | | | | VDGVEVHNAKTKPREEQYNSTYRVVSVLTVL | VQWKVDNALQSGNSQE |
| | | | | HQDWLNGKEYKCKVSNKALPAPIEKTISKAKG | SVTEQDSKDSTYSLSS |
| | | | | QPREPQVYTLPPSREEMTKNQVSLTCLVKGF | TLTLSKADYEKHKVYA |
| | | | | YPSDIAVEWESNGQPENNYKTTPPVLDSDGS | CEVTHQGLSSPVTKSF |
| | | | | FFLYSKLTVDKSRWQQGNVFSCSVMHEALHN | NRGEC |
| | | | | HYTQKSLSLSPGK | |
| CD278 | alomfi- | 111 | 112 | EVQLVESGGGVVRPGGSLRLSCVASGVTFDD | EIVLTQSPGTLSLSPG |
| (ICOS) | limab | | | YGMSWVRQAPGKGLEWVSGINWNGGDTDYS | ERATLSCRASQSVSRS |
| | | | | DSVKGRFTISRDNAKNSLYLQMNSLRAEDTAL | YLAWYQQKRGQAPRLL |
| | | | | YYCARDFYGSGSYYHVPFDYWGQGILVTVSS | IYGASSRATGIPDRFS |
| | | | | ASTKGPSVFPLAPSSKSTSGGTAALGCLVKDY | GDGSGTDFTLSISRLE |
| | | | | FPEPVTVSWNSGALTSGVHTFPAVLQSSGLY | PEDFAVYYCHQYDMSP |
| | | | | SLSSVVTVPSSSLGTQTYICNVNHKPSNTKVD | FTFGPGTKVDIKRTVA |
| | | | | KKVEPKSCDKTHTCPPCPAPELLGGPSVFLFP | APSVFIFPPSDEQLKS |
| | | | | PKPKDTLMISRTPEVTCVVVDVSHEDPEVKFN | GTASVVCLLNNFYPRE |
| | | | | WYVDGVEVHNAKTKPREEQYNSTYRVVSVLT | AKVQWKVDNALQSGNS |
| | | | | VLHQDWLNGKEYKCKVSNKALPAPIEKTISKA | QESVTEQDSKDSTYSL |
| | | | | KGQPREPQVYTLPPSRDELTKNQVSLTCLVK | SSTLTLSKADYEKHKV |
| | | | | GFYPSDIAVEWESNGQPENNYKTTPPVLDSD | YACEVTHQGLSSPVTK |
| | | | | GSFFLYSKLTVDKSRWQQGNVFSCSVMHEAL | SFNRGEC |
| | | | | HNHYTQKSLSLSPGK | |
| CD278 | feladi- | 113 | 114 | QVQLVQSGAEVKKPGSSVKVSCKASGYTFTD | EIVLTQSPATLSLSPG |
| | limab | | | YAMHWVRQAPGQGLEWMGLISIYSDHTNYNQ | ERATLSCSASSSVSYM |
| | | | | KFQGRVTITADKSTSTAYMELSSLRSEDTAVY | HWYQQKPGQAPRLLIY |
| | | | | YCGRNNYGNYGWYFDVWGQGTTVTVSSAST | DTSKLASGIPARFSGS |
| | | | | KGPSVFPLAPCSRSTSESTAALGCLVKDYFPE | GSGTDYTLTISSLEPE |
| | | | | PVTVSWNSGALTSGVHTFPAVLQSSGLYSLSS | DFAVYYCFQGSGYPYT |
| | | | | VVTVPSSSLGTKTYTCNVDHKPSNTKVDKRVE | FGQGTKLEIKRTVAAP |
| | | | | SKYGPPCPPCPAPEFEGGPSVFLFPPKPKDTL | SVFIFPPSDEQLKSGT |
| | | | | MISRTPEVTCVVVDVSQEDPEVQFNWYVDGV | ASVVCLLNNFYPREAK |
| | | | | EVHNAKTKPREEQFNSTYRVVSVLTVLHQDW | VQWKVDNALQSGNSQE |
| | | | | LNGKEYKCKVSNKGLPSSIEKTISKAKGQPRE | SVTEQDSKDSTYSLSS |
| | | | | PQVYTLPPSQEEMTKNQVSLTCLVKGFYPSDI | TLTLSKADYEKHKVYA |
| | | | | AVEWESNGQPENNYKTTPPVLDSDGSFFLYS | CEVTHQGLSSPVTKSF |
| | | | | RLTVDKSRWQEGNVFSCSVMHEALHNHYTQ | NRGEC |
| | | | | KSLSLSLGK | |
| CD278 | izuralimab | 115 | 116 | QVQLVQSGAEVKKPGASVKVSCKASGYTFTG | DIQMTQSPSSVSASVG |
| | | | | YYMHWVRQAPGQGLEWMGWINPHSGGTNY | DRVTITCRASQGISRL |
| | | | | AQKFQGRVTMTRDTSISTAYMELSRLRSDDTA | LAWYQQKPGKAPKLLI |
| | | | | VYYCARTYYYDSSGYYHDAFDIWGQGTMVTV | YVASSLQSGVPSRFSG |
| | | | | SSASTKGPSVFPLAPSSKSTSGGTAALGCLVK | SGSGTDFTLTISSLQP |
| | | | | DYFPEPVTVSWNSGALTSGVHTFPAVLQSSG | EDFATYYCQQANSFPW |
| | | | | LYSLSSVVTVPSSSLGTQTYICNVNHKPSDTK | TFGQGTKVEIKRTVAA |
| | | | | VDKKVEPKSCDKTHTCPPCPAPPVAGPSVFLF | PSVFIFPPSDEQLKSG |
| | | | | PPKPKDTLMISRTPEVTCVVVDVKHEDPEVKF | TASVVCLLNNFYPREA |
| | | | | NWYVDGVEVHNAKTKPREEEYNSTYRVVSVL | KVQWKVDNALQSGNSQ |
| | | | | TVLHQDWLNGKEYKCKVSNKALPAPIEKTISK | ESVTEQDSKDSTYSLS |
| | | | | AKGQPREPQVYTLPPSREEMTKNQVSLTCDV | STLTLSKADYEKHKVY |
| | | | | SGFYPSDIAVEWESDGQPENNYKTTPPVLDS | ACEVTHQGLSSPVTKS |
| | | | | DGSFFLYSKLTVDKSRWEQGDVFSCSVLHEA | FNRGEC |
| | | | | LHSHYTQKSLSLSPGK | |
| CD278 | MEDI-570 | 117 | 118 | QVQLVQSGAEVKKPGASVKVSCKASGYTFTG | DIQMTQSPSSVSASVG |
| | | | | YYMHWVRQAPGQGLEWMGWINPHSGGTNY | DRVTITCRASQGISRL |
| | | | | AQKFQGRVTMTRDTSISTAYMELSRLRSDDTA | LAWYQQKPGKAPKLLI |
| | | | | VYYCARTYYYDSSGYYHDAFDIWGQGTMVTV | YVASSLQSGVPSRFSG |
| | | | | SS | SGSGTDFTLTISSLQP |
| | | | | | EDFATYYCQQANSFPW |
| | | | | | TFGQGTKVEIK |
| CD278 | voprate- | 119 | 120 | EVQLVESGGGLVQPGGSLRLSCAASGFTFSD | DIVMTQSPDSLAVSLG |
| | limab | | | YWMDWVRQAPGKGLVWVSNIDEDGSITEYSP | ERATINCKSSQSLLSG |
| | | | | FVKGRFTISRDNAKNTLYLQMNSLRAEDTAVY | SFNYLTWYQQKPGQPP |
| | | | | YCTRWGRFGFDSWGQGTLVTVSSASTKGPS | KLLIFYASTRHTGVPD |
| | | | | VFPLAPSSKSTSGGTAALGCLVKDYFPEPVTV | RFSGSGSGTDFTLTIS |
| | | | | SWNSGALTSGVHTFPAVLQSSGLYSLSSVVTV | SLQAEDVAVYYCHHHY |
| | | | | PSSSLGTQTYICNVNHKPSNTKVDKKVEPKSC | NAPPTFGPGTKVDIKR |
| | | | | DKTHTCPPCPAPELLGGPSVFLFPPKPKDTLM | TVAAPSVFIFPPSDEQ |
| | | | | ISRTPEVTCVVVDVSHEDPEVKFNWYVDGVE | LKSGTASVVCLLNNFY |
| | | | | VHNAKTKPREEQYNSTYRVVSVLTVLHQDWL | PREAKVQWKVDNALQS |
| | | | | NGKEYKCKVSNKALPAPIEKTISKAKGQPREP | GNSQESVTEQDSKDST |
| | | | | QVYTLPPSREEMTKNQVSLTCLVKGFYPSDIA | YSLSSTLTLSKADYEK |
| | | | | VEWESNGQPENNYKTTPPVLDSDGSFFLYSK | HKVYACEVTHQGLSSP |
| | | | | LTVDKSRWQQGNVFSCSVMHEALHNHYTQK | VTKSFNRGEC |
| | | | | SLSLSPGK | |
| CD278 | acazicol- | 121 | | DTQEKEVRAMVGSDVELSCACPEGSRFDLND | |
| | cept | | | VYVYWQTSESKTVVTYHIPQHSSLEYVDSRYR | |
| | | | | NRALMSPAGMLRGDFSLRLFNVTPQDEQKFH | |
| | | | | CLVLSRSLGFQEVLSVEVTLHVAANFSVGGGG | |
| | | | | SGGGGSEPKSSDKTHTCPPCPAPEAEGAPSV | |
| | | | | FLFPPKPKDTLMISRTPEVTCVVVDVSHEDPE | |
| | | | | VKFNWYVDGVEVHNAKTKPREEQYNSTYRVV | |
| | | | | SVLTVLHQDWLNGKEYKCKVSNKALPAPIEKTI | |
| | | | | SKAKGQPREPQVYTLPPSRDELTKNQVSLTCL | |
| | | | | VKGFYPSDIAVEWESNGQPENNYKTTPPVLD | |
| | | | | SDGSFFLYSKLTVDKSRWQQGNVFSCSVMHE | |
| | | | | ALHNHYTQKSLSLSPG | |
| CD28 | FR104CD- | 122 | 123 | VQLQQSGAELKKPGASVKVSCKASGYTFTEYI | DIQMTQSPSSLSASVG |
| | CD28 | | | IHWIKLRSGQGLEWIGWFYPGSNDIQYNAQFK | DRVTITCKTNENIYSN |
| | binding | | | GKATLTADKSSSTVYMELTGLTPEDSAVYFCA | LAWYQQKDGKSPQLLI |
| | fragment | | | RRDDFSGYDALPYWGQGTLVTVSA | YAATHLVEGVPSRFSG |
| | | | | | SGSGTQYSLTISSLQP |
| | | | | | EDFGNYYCQHFWGTPC |
| | | | | | TFGGGTKLEIKR |
| CD28 | Lulizumab- | 124 | | DIQMTQSPSSLSASVGDRVTITCRASRPIWPFL | |
| | domain | | | EWYQQKPGKAPKLLIYFTSRLRHGVPSRFSGS | |
| | antagonist | | | GSGTCFTLTISSLQPEDFATYYCLQNVANPAT | |
| | antibody | | | FSQGTKVEIKR | |
| CD28 | prezalumab | 125 | 126 | EVQLVESGGGLVQPGGSLRLSCAASGFTFSS | DIQMTQSPSSLSASVG |
| | | | | YWMSWVRQAPGKGLEWVAYIKQDGNEKYYV | DRVTITCRASQGISNW |
| | | | | DSVKGRFTISRDNAKNSLYLQMNSLRAEDTAV | LAWYQQKPEKAPKSLI |
| | | | | YYCAREGILWFGDLPTFWGQGTLVTVSSAST | YAASSLQSGVPSRFSG |
| | | | | KGPSVFPLAPCSRSTSESTAALGCLVKDYFPE | SGSGTDFTLTISSLQP |
| | | | | PVTVSWNSGALTSGVHTFPAVLQSSGLYSLSS | EDFATYYCQQYDSYPR |
| | | | | VVTVPSSNFGTQTYTCNVDHKPSNTKVDKTV | TFGQGTKVEIKRTVAA |
| | | | | ERKCCVECPPCPAPPVAGPSVFLFPPKPKDTL | PSVFIFPPSDEQLKSG |
| | | | | MISRTPEVTCVVVDVSHEDPEVQFNWYVDGV | TASVVCLLNNFYPREA |
| | | | | EVHNAKTKPREEQFNSTFRVVSVLTVVHQDW | KVQWKVDNALQSGNSQ |
| | | | | LNGKEYKCKVSNKGLPAPIEKTISKTKGQPRE | ESVTEQDSKDSTYSLS |
| | | | | PQVYTLPPSREEMTKNQVSLTCLVKGFYPSDI | STLTLSKADYEKHKVY |
| | | | | AVEWESNGQPENNYKTTPPMLDSDGSFFLYS | ACEVTHQGLSSPVTKS |
| | | | | KLTVDKSRWQQGNVFSCSVMHEALHNHYTQ | FNRGEC |
| | | | | KSLSLSPGK | |
| CD28 | TGN1412 | 127 | 128 | QVQLVESGGGVVQPGRSLRLSCAASGFTESS | DIQMTQSPSSLSASVG |
| | (Therali- | | | YDMHWVRQAPGKGLEWVAVIWYDGSNKYYA | DRVTITCRASQGISRW |
| | zumab) | | | DSVKGRFTISRDNSKNTLYLQMNSLRAEDTAV | LAWYQQKPEKAPKSLI |
| | | | | YYCARGSGNWGFFDYWGQGTLVTVSSASTK | YAASSLQSGVPSRFSG |
| | | | | GPSVFPLAPSSKSTSGGTAALGCLVKDYFPEP | SGSGTDFTLTISSLQP |
| | | | | VTVSWNSGALTSGVHTFPAVLQSSGLYSLSSV | EDFATYYCQQYNTYPR |
| | | | | VTVPSSSLGTQTYICNVNHKPSNTKVDKKVEP | TFGQGTKVEIKRTVAA |
| | | | | KSCDKTHTCPPCPAPELLGGPSVFLFPPKPKD | PSVFIFPPSDEQLKSG |
| | | | | TLMISRTPEVTCVVVDVSHEDPEVKFNWYVD | TASVVCLLNNFYPREA |
| | | | | GVEVHNAKTKPREEQYNSTYRVVSVLTVLHQ | KVQWKVDNALQSGNSQ |
| | | | | DWLNGKEYKCKVSNKALPAPIEKTISKAKGQP | ESVTEQDSKDSTYSLS |
| | | | | REPQVYTLPPSRDELTKNQVSLTCLVKGFYPS | STLTLSKADYEKHKVY |
| | | | | DIAVEWESNGQPENNYKTTPPVLDSDGSFFLY | ACEVTHQGLSSPVTKS |
| | | | | SKLTVDKSRWQQGNVFSCSVMHEALHNHYT | FNRGEC |
| | | | | QKSLSLSPGKGSS | |
| CD28/ | davoceti- | 129 | | VIHVTKEVKEVATLSCGYNVSVEELEQTRIYW | |
| PD- | cept | | | QKDKKMVLTMMSGDLNIWPEYKNRTIFDITNN | |
| L1/CTL | | | | LSIMILGLRPSDEGTYECVVLKYEKGAFKREHL | |
| A-4 | | | | AEVTLSVKADGSGGGGSEPKSSDKTHTCPPC | |
| | | | | PAPEAEGAPSVFLFPPKPKDTLMISRTPEVTC | |
| | | | | VVVDVSHEDPEVKFNWYVDGVEVHNAKTKPR | |
| | | | | EEQYNSTYRVVSVLTVLHQDWLNGKEYKCKV | |
| | | | | SNKALPAPIEKTISKAKGQPREPQVYTLPPSRD | |
| | | | | ELTKNQVSLTCLVKGFYPSDIAVEWESNGQPE | |
| | | | | NNYKTTPPVLDSDGSFFLYSKLTVDKSRWQQ | |
| | | | | GNVFSCSVMHEALHNHYTQKSLSLSPG | |
| CD3 | 40G5cv- | 130 | 131 | QVQLQQSGPELVKPGASVKISCKASGYTFTNY | DIVMTQSPSSLAVSAG |
| | CD3 | | | YIHWVKQRPGQGLEWIGWIYPGDGNTKYNEK | EKVTMSCKSSQSLLNS |
| | binding | | | FKGKATLTADTSSSTAYMQLSSLTSEDSAVYY | RTRKNYLAWYQQKPGQ |
| | fragment | | | CARDSYSNYYFDYWGQGTTLTVSS | SPKLLIYWASTRESGV |
| | | | | | PDRFTGSGSGTDFTLT |
| | | | | | ISSVQAEDLAVYYCTQ |
| | | | | | SFILRTFGGGTKLEIK |
| CD3E | Acapatamab | 132 | | EVQLVESGGGLVQPGGSLKLSCAASGFTFNK | |
| | | | | YAMNWVRQAPGKGLEWVARIRSKYNNYATYY | |
| | | | | ADSVKDRFTISRDDSKNTAYLQMNNLKTEDTA | |
| | | | | VYYCVRHGNFGNSYISYWAYWGQGTLVTVSS | |
| | | | | GGGGSGGGGSGGGGSQTVVTQEPSLTVSPG | |
| | | | | GTVTLTCGSSTGAVTSGNYPNWVQQKPGQA | |
| | | | | PRGLIGGTKFLAPGTPARFSGSLLGGKAALTL | |
| | | | | SGVQPEDEAEYYCVLWYSNRWVFGGGTKLT | |
| | | | | VL | |
| CD3 | Alnuc- | 133 | 134 | EVQLLESGGGLVQPGGSLRLSCAASGFTFSD | EVQLLESGGGLVQPGG |
| | tamab- | | | NAMGWVRQAPGKGLEWVSAISGPGSSTYYA | SLRLSCAASGFTFSTY |
| | First | | | DSVKGRFTISRDNSKNTLYLQMNSLRAEDTAV | AMNWVRQAPGKGLEWV |
| | heavy | | | YYCAKVLGWFDYWGQGTLVTVSSASTKGPSV | SRIRSKYNNYATYYAD |
| | chain | | | FPLAPSSKSTSGGTAALGCLVEDYFPEPVTVS | SVKGRFTISRDDSKNT |
| | and | | | WNSGALTSGVHTFPAVLQSSGLYSLSSVVTVP | LYLQMNSLRAEDTAVY |
| | second | | | SSSLGTQTYICNVNHKPSNTKVDEKVEPKSCD | YCVRHGNFGNSYVSWF |
| | light | | | KTHTCPPCPAPEAAGGPSVFLFPPKPKDTLMI | AYWGQGTLVTVSSASV |
| | chain. | | | SRTPEVTCVVVDVSHEDPEVKFNWYVDGVEV | AAPSVFIFPPSDEQLK |
| | | | | HNAKTKPREEQYNSTYRVVSVLTVLHQDWLN | SGTASVVCLLNNFYPR |
| | | | | GKEYKCKVSNKALGAPIEKTISKAKGQPREPQ | EAKVQWKVDNALQSGN |
| | | | | VCTLPPSRDELTKNQVSLSCAVKGFYPSDIAV | SQESVTEQDSKDSTYS |
| | | | | EWESNGQPENNYKTTPPVLDSDGSFFLVSKL | LSSTLTLSKADYEKHK |
| | | | | TVDKSRWQQGNVFSCSVMHEALHNHYTQKS | VYACEVTHQGLSSPVT |
| | | | | LSLSPGK | KSFNRGEC |
| CD3E | blinatu- | 135 | | DIQLTQSPASLAVSLGQRATISCKASQSVDYD | |
| | momab- | | | GDSYLNWYQQIPGQPPKLLIYDASNLVSGIPP | |
| | scFv-scFv | | | RFSGSGSGTDFTLNIHPVEKVDAATYHCQQST | |
| | anti-CD3E | | | EDPWTFGGGTKLEIKGGGGSGGGGSGGGGS | |
| | and anti- | | | QVQLQQSGAELVRPGSSVKISCKASGYAFSS | |
| | CD19 | | | YWMNWVKQRPGQGLEWIGQIWPGDGDTNY | |
| | | | | NGKFKGKATLTADESSSTAYMQLSSLASEDSA | |
| | | | | VYFCARRETTTVGRYYYAMDYWGQGTTVTVS | |
| | | | | SGGGGSDIKLQQSGAELARPGASVKMSCKTS | |
| | | | | GYTFTRYTMHWVKQRPGQGLEWIGYINPSRG | |
| | | | | YTNYNQKFKDKATLTTDKSSSTAYMQLSSLTS | |
| | | | | EDSAVYYCARYYDDHYCLDYWGQGTTLTVSS | |
| | | | | VEGGSGGSGGSGGSGGVDDIQLTQSPAIMSA | |
| | | | | SPGEKVTMTCRASSSVSYMNWYQQKSGTSP | |
| | | | | KRWIYDTSKVASGVPYRFSGSGSGTSYSLTIS | |
| | | | | SMEAEDAATYYCQQWSSNPLTFGAGTKLEL | |
| | | | | KHHHHHH | |
| CD3 | cevostamab | 136 | 137 | EVQLVQSGAEVKKPGASVKVSCKASGFTFTS | DIVMTQSPDSLAVSLG |
| | | | | YYIHWVRQAPGQGLEWIGWIYPENDNTKYNE | ERATINCKSSQSLLNS |
| | | | | KFKDRVTITADTSTSTAYLELSSLRSEDTAVYY | RTRKNYLAWYQQKPGQ |
| | | | | CARDGYSRYYFDYWGQGTLVTVSSASTKGPS | SPKLLIYWTSTRKSGV |
| | | | | VFPLAPSSKSTSGGTAALGCLVKDYFPEPVTV | PDRFSGSGSGTDFTLT |
| | | | | SWNSGALTSGVHTFPAVLQSSGLYSLSSVVTV | ISSLQAEDVAVYYCKQ |
| | | | | PSSSLGTQTYICNVNHKPSNTKVDKKVEPKSC | SFILRTFGQGTKVEIK |
| | | | | DKTHTCPPCPAPELLGGPSVFLFPPKPKDTLM | RTVAAPSVFIFPPSDE |
| | | | | ISRTPEVTCVVVDVSHEDPEVKFNWYVDGVE | QLKSGTASVVCLLNNF |
| | | | | VHNAKTKPREEQYGSTYRVVSVLTVLHQDWL | YPREAKVQWKVDNALQ |
| | | | | NGKEYKCKVSNKALPAPIEKTISKAKGQPREP | SGNSQESVTEQDSKDS |
| | | | | QVYTLPPSREEMTKNQVSLSCAVKGFYPSDIA | TYSLSSTLTLSKADYE |
| | | | | VEWESNGQPENNYKTTPPVLDSDGSFFLVSK | KHKVYACEVTHQGLSS |
| | | | | LTVDKSRWQQGNVFSCSVMHEALHNHYTQK | PVTKSFNRGEC |
| | | | | SLSLSPGK | |
| CD3E | cibisa- | 138 | 139 | QVQLVQSGAEVKKPGASVKVSCKASGYTFTE | QAVVTQEPSLTVSPGG |
| | tamab | | | FGMNWVRQAPGQGLEWMGWINTKTGEATYV | TVTLTCGSSTGAVTTS |
| | | | | EEFKGRVTFTTDTSTSTAYMELRSLRSDDTAV | NYANWVQEKPGQAFRG |
| | | | | YYCARWDFAYYVEAMDYWGQGTTVTVSSAT | LIGGTNKRAPGTPARF |
| | | | | KGPSVFPLAPSSKSTSGGTAALGCLVKDYFPE | SGSLLGGKAALTLSGA |
| | | | | PVTVSWNSGALTSGVHTFPAVLQSSGLYSLSS | QPEDEAEYYCALWYSN |
| | | | | VVTVPSSSLGTQTYICNVNHKPSNTKVDKKVE | LWVFGGGTKLTVLSSA |
| | | | | PKSCDGGGGSGGGGSEVQLLESGGGLVQPG | STKGPSVFPLAPSSKS |
| | | | | GSLRLSCAASGFTFSTYAMNWVRQAPGKGLE | TSGGTAALGCLVKDYF |
| | | | | WVSRIRSKYNNYATYYADSVKGRFTISRDDSK | PEPVTVSWNSGALTSG |
| | | | | NTLYLQMNSLRAEDTAVYYCVRHGNFGNSYV | VHTFPAVLQSSGLYSL |
| | | | | SWFAYWGQGTLVTVSSASVAAPSVFIFPPSDE | SSVVTVPSSSLGTQTY |
| | | | | QLKSGTASVVCLLNNFYPREAKVQWKVDNAL | ICNVNHKPSNTKVDKK |
| | | | | QSGNSQESVTEQDSKDSTYSLSSTLTLSKADY | VEPKSC |
| | | | | EKHKVYACEVTHQGLSSPVTKSFNRGECDKT | |
| | | | | HTCPPCPAPEAAGGPSVFLFPPKPKDTLMISR | |
| | | | | TPEVTCVVVDVSHEDPEVKFNWYVDGVEVHN | |
| | | | | AKTKPREEQYNSTYRVVSVLTVLHQDWLNGK | |
| | | | | EYKCKVSNKALGAPIEKTISKAKGQPREPQVY | |
| | | | | TLPPCRDELTKNQVSLWCLVKGFYPSDIAVEW | |
| | | | | ESNGQPENNYKTTPPVLDSDGSFFLYSKLTVD | |
| | | | | KSRWQQGNVFSCSVMHEALHNHYTQKSLSLS | |
| | | | | PGK | |
| CD3E | dafsolimab | 140 | 141 | QVQLQQSGAELARPGASVKMSCKASGYTFTS | QIVLTQSPAIMSASPG |
| | | | | YTMHWVKQRPGQGLEWIGYINPSSGYTNYIQ | EKVTMTCSASSSVSYM |
| | | | | RFKDKATLTADKSSSTAYMQVSSLTSEDSAVY | HWYQQKSGTSPKRWIY |
| | | | | YCARGSRYDYYGMDYWGQGTSVTVSSAKTT | DTSKLASGVPARFSGS |
| | | | | PPSVYPLAPGCGDTTGSSVTLGCLVKGYFPES | GSGTSYSLTISSMEAE |
| | | | | VTVTWNSGSLSSSVHTFPALLQSGLYTMSSSV | DAATYYCQQWSSNPLT |
| | | | | TVPSSTWPSQTVTCSVAHPASSTTVDKKLEPS | FGAGTKLELKRADAAP |
| | | | | GPISTINPCPPCKECHKCPAPNLEGGPSVFIFP | TVSIFPPSSEQLTSGG |
| | | | | PNIKDVLMISLTPKVTCVVVDVSEDDPDVQISW | ASVVCFLNNFYPKDIN |
| | | | | FVNNVEVHTAQTQTHREDYNSTIRVVSTLPIQ | VKWKIDGSERQNGVLN |
| | | | | HQDWMSGKEFKCKVNNKDLPSPIERTISKIKG | SWTDQDSKDSTYSMSS |
| | | | | LVRAPQVYILPPPAEQLSRKDVSLTCLVVGFN | TLTLTKDEYERHNSYT |
| | | | | PGDISVEWTSNGHTEENYKDTAPVLDSDGSY | CEATHKTSTSPIVKSF |
| | | | | FIYSKLNMKTSKWEKTDSFSCNVRHEGLKNYY | NRNEC |
| | | | | LKKTISRSPGK | |
| CD3E | Duvortux- | 142 | 143 | ENVLTQSPATLSVTPGEKATITCRASQSVSYM | QAVVTQEPSLTVSPGG |
| | izumab- | | | HWYQQKPGQAPRLLIYDASNRASGVPSRFSG | TVTLTCRSSTGAVTTS |
| | bispecific | | | SGSGTDHTLTISSLEAEDAATYYCFQGSVYPF | NYANWVQQKPGQAPRG |
| | anti-CD3E | | | TFGQGTKLEIKGGGSGGGGEVQLVESGGGLV | LIGGTNKRAPWTPARF |
| | and | | | QPGGSLRLSCAASGFTFSTYAMNWVRQAPG | SGSLLGGKAALTITGA |
| | anti-CD19 | | | KGLEWVGRIRSKYNNYATYYADSVKGRFTISR | QAEDEADYYCALWYSN |
| | | | | DDSKNSLYLQMNSLKTEDTAVYYCVRHGNFG | LWVFGGGTKLTVLGGG |
| | | | | NSYVSWFAYWGQGTLVTVSSASTKGEVAACE | GSGGGGQVTLRESGPA |
| | | | | KEVAALEKEVAALEKEVAALEKGGGDKTHTCP | LVKPTQTLTLTCTFSG |
| | | | | PCPAPEAAGGPSVFLFPPKPKDTLMISRTPEV | FSLSTSGMGVGWIRQP |
| | | | | TCVVVDVSHEDPEVKFNWYVDGVEVHNAKTK | PGKALEWLAHIWWDDD |
| | | | | PREEQYNSTYRVVSVLTVLHQDWLNGKEYKC | KRYNPALKSRLTISKD |
| | | | | KVSNKALPAPIEKTISKAKGQPREPQVYTLPPS | TSKNQVFLTMTNMDPV |
| | | | | REEMTKNQVSLWCLVKGFYPSDIAVEWESNG | DTATYYCARMELWSYY |
| | | | | QPENNYKTTPPVLDSDGSFFLYSKLTVDKSR | FDYWGQGTTVTVSSAS |
| | | | | WQQGNVFSCSVMHEALHNHYTQKSLSLSPG | TKGKVAACKEKVAALK |
| | | | | K | EKVALKEKVAALKE |
| CD3 | elrana- | 144 | 145 | EVQLVESGGGLVQPGGSLRLSCAASGFTFSD | DIVMTQSPDSLAVSLG |
| | tamab | | | YYMTWVRQAPGKGLEWVAFIRNRARGYTSD | ERATINCKSSQSLFNV |
| | | | | HNPSVKGRFTISRDNAKNSLYLQMNSLRAEDT | RSRKNYLAWYQQKPGQ |
| | | | | AVYYCARDRPSYYVLDYWGQGTTVTVSSAST | PPKLLISWASTRESGV |
| | | | | KGPSVFPLAPCSRSTSESTAALGCLVKDYFPE | PDRFSGSGSGTDFTLT |
| | | | | PVTVSWNSGALTSGVHTFPAVLQSSGLYSLSS | ISSLQAEDVAVYYCKQ |
| | | | | VVTVPSSNFGTQTYTCNVDHKPSNTKVDKTV | SYDLFTFGSGTKLEIK |
| | | | | ERKCRVRCPRCPAPPVAGPSVFLFPPKPKDTL | RTVAAPSVFIFPPSDE |
| | | | | MISRTPEVTCVVVAVSHEDPEVQFNWYVDGV | QLKSGTASVVCLLNNF |
| | | | | EVHNAKTKPREEQFNSTFRVVSVLTVVHQDW | YPREAKVQWKVDNALQ |
| | | | | LNGKEYKCKVSNKGLPSSIEKTISKTKGQPRE | SGNSQESVTEQDSKDS |
| | | | | PQVYTLPPSREEMTKNQVSLTCLVKGFYPSDI | TYSLSSTLTLSKADYE |
| | | | | AVEWESNGQPENNYKTTPPMLDSDGSFFLYS | KHKVYACEVTHQGLSS |
| | | | | RLTVDKSRWQQGNVFSCSVMHEALHNHYTQ | PVTKSFNRGEC |
| | | | | KSLSLSPGK | |
| CD3E | eluvix- | 146 | | QVQLVQSGAEVKKPGESVKVSCKASGYTFTN | |
| | tamab | | | YGMNWVKQAPGQGLEWMGWINTYTGEPTYA | |
| | | | | DKFQGRVTMTTDTSTSTAYMEIRNLGGDDTA | |
| | | | | VYYCARWSWSDGYYVYFDYWGQGTSVTVSS | |
| | | | | GGGGSGGGGSGGGGSDIVMTQSPDSLTVSL | |
| | | | | GERTTINCKSSQSVLDSSTNKNSLAWYQQKP | |
| | | | | GQPPKLLLSWASTRESGIPDRFSGSGSGTDFT | |
| | | | | LTIDSPQPEDSATYYCQQSAHFPITFGQGTRL | |
| | | | | EIKSGGGGSEVQLVESGGGLVQPGGSLKLSC | |
| | | | | AASGFTFNKYAMNWVRQAPGKGLEWVARIRS | |
| | | | | KYNNYATYYADSVKDRFTISRDDSKNTAYLQM | |
| | | | | NNLKTEDTAVYYCVRHGNFGNSYISYWAYWG | |
| | | | | QGTLVTVSSGGGGSGGGGSGGGGSQTVVTQ | |
| | | | | EPSLTVSPGGTVTLTCGSSTGAVTSGNYPNW | |
| | | | | VQQKPGQAPRGLIGGTKFLAPGTPARFSGSLL | |
| | | | | GGKAALTLSGVQPEDEAEYYCVLWYSNRWVF | |
| | | | | GGGTKLTVLHHHHHH | |
| CD3E | emerfe- | 147 | | QVQLVQSGAEVKKPGESVKVSCKASGYTFTN | |
| | tamab | | | YGMNWVKQAPGQCLEWMGWINTYTGEPTYA | |
| | | | | DKFQGRVTMTTDTSTSTAYMEIRNLGGDDTA | |
| | | | | VYYCARWSWSDGYYVYFDYWGQGTSVTVSS | |
| | | | | GGGGSGGGGSGGGGSDIVMTQSPDSLTVSL | |
| | | | | GERTTINCKSSQSVLDSSTNKNSLAWYQQKP | |
| | | | | GQPPKLLLSWASTRESGIPDRFSGSGSGTDFT | |
| | | | | LTIDSPQPEDSATYYCQQSAHFPITFGCGTRL | |
| | | | | EIKSGGGGSEVQLVESGGGLVQPGGSLKLSC | |
| | | | | AASGFTFNKYAMNWVRQAPGKGLEWVARIRS | |
| | | | | KYNNYATYYADSVKDRFTISRDDSKNTAYLQM | |
| | | | | NNLKTEDTAVYYCVRHGNFGNSYISYWAYWG | |
| | | | | QGTLVTVSSGGGGSGGGGSGGGGSQTVVTQ | |
| | | | | EPSLTVSPGGTVTLTCGSSTGAVTSGNYPNW | |
| | | | | VQQKPGQAPRGLIGGTKFLAPGTPARFSGSLL | |
| | | | | GGKAALTLSGVQPEDEAEYYCVLWYSNRWVF | |
| | | | | GGGTKLTVLGGGGDKTHTCPPCPAPELLGGP | |
| | | | | SVFLFPPKPKDTLMISRTPEVTCVVVDVSHED | |
| | | | | PEVKFNWYVDGVEVHNAKTKPCEEQYGSTYR | |
| | | | | CVSVLTVLHQDWLNGKEYKCKVSNKALPAPIE | |
| | | | | KTISKAKGQPREPQVYTLPPSREEMTKNQVSL | |
| | | | | TCLVKGFYPSDIAVEWESNGQPENNYKTTPPV | |
| | | | | LDSDGSFFLYSKLTVDKSRWQQGNVFSCSVM | |
| | | | | HEALHNHYTQKSLSLSPGKGGGGSGGGGSG | |
| | | | | GGGSGGGGSGGGGSGGGGSDKTHTCPPCP | |
| | | | | APELLGGPSVFLFPPKPKDTLMISRTPEVTCVV | |
| | | | | VDVSHEDPEVKFNWYVDGVEVHNAKTKPCEE | |
| | | | | QYGSTYRCVSVLTVLHQDWLNGKEYKCKVSN | |
| | | | | KALPAPIEKTISKAKGQPREPQVYTLPPSREEM | |
| | | | | TKNQVSLTCLVKGFYPSDIAVEWESNGQPEN | |
| | | | | NYKTTPPVLDSDGSFFLYSKLTVDKSRWQQG | |
| | | | | NVFSCSVMHEALHNHYTQKSLSLSPGK | |
| CD3 | emfiza- | 148 | 149 | DVVMTQSPSTLSASVGDRVTINCQASESISSW | AIQLTQSPSSLSASVG |
| | tamab | | | LAWYQQKPGKAPKLLIYEASKLASGVPSRFS | DRVTITCRASQGISSA |
| | | | | GSGSGTEFTLTISSLQPDDFATYYCQGYFYFI | LAWYQQKPGKAPKLLI |
| | | | | SRTYVNSFGGGTKVEIKGGGGSGGGGSGGG | YDASSLESGVPSRFSG |
| | | | | GSGGGGSEVQLVESGGGLVQPGGSLRLSCA | SGSGTDFTLTISSLQP |
| | | | | ASGFTISTNAMSWVRQAPGKGLEWIGVITGRD | EDFATYYCQQFNSYPF |
| | | | | ITYYASWAKGRFTISRDNSKNTLYLQMNSLRA | TFGPGTKVDIKRTVAA |
| | | | | EDTAVYYCARDGGSSAITSNNIWGQGTLVTVS | PSVFIFPPSDEQLKSG |
| | | | | SGGGGSGGGGSEVQLVQSGAEVKKPGESLKI | TASVVCLLNNFYPREA |
| | | | | SCKGSGYSFSSSWIGWVRQAPGKGLEWMGII | KVQWKVDNALQSGNSQ |
| | | | | YPDDSDTRYSPSFQGQVTISADKSIRTAYLQW | ESVTEQDSKDSTYSLS |
| | | | | SSLKASDTAMYYCARHVTMIWGVIIDFWGQGT | STLTLSKADYEKHKVY |
| | | | | LVTVSSASTKGPSVFPLAPSSKSTSGGTAALG | ACEVTHQGLSSPVTKS |
| | | | | CLVKDYFPEPVTVSWNSGALTSGVHTFPAVL | FNRGEC |
| | | | | QSSGLYSLSSVVTVPSSSLGTQTYICNVNHKP | |
| | | | | SNTKVDKRVEPKSCDKTHTCPPCPAPEAAGA | |
| | | | | PSVFLFPPKPKDTLMISRTPEVTCVVVDVSHE | |
| | | | | DPEVKFNWYVDGVEVHNAKTKPREEQYNSTY | |
| | | | | RVVSVLTVLHQDWLNGKEYKCAVSNKALPAPI | |
| | | | | EKTISKAKGQPREPQVYTLPPSRDELTKNQVS | |
| | | | | LTCLVKGFYPSDIAVEWESNGQPENNYKTTPP | |
| | | | | VLDSDGSFFLYSKLTVDKSRWQQGNVFSCSV | |
| | | | | MHEALHNHYTQKSLSLSPGGGGGSGGGGSE | |
| | | | | VQLLESGGGLVQPGGSLRLSCAASGFSFSSG | |
| | | | | YDMCWVRQAPGKGLEWIACIAAGSAGITYDA | |
| | | | | NWAKGRFTISRDNSKNTLYLQMNSLRAEDTA | |
| | | | | VYYCARSAFSFDYAMDLWGQGTLVTVSSGG | |
| | | | | GGSGGGGSGGGGSGGGGSDIQMTQSPSTLS | |
| | | | | ASVGDRVTITCQASQSISSHLNWYQQKPGKA | |
| | | | | PKLLIYKASTLASGVPSRFSGSGSGTEFTLTIS | |
| | | | | SLQPDDFATYYCQQGYSWGNVDNVFGGGTK | |
| | | | | VEIKGGGGSGGGGSRSLVESGGGLVQPGGS | |
| | | | | LRLSCTASGFTISSYHMQWVRQAPGKGLEYIG | |
| | | | | TISSGGNVYYASSARGRFTISRPSSKNTVDLQ | |
| | | | | MNSLRAEDTAVYYCARDSGYSDPMWGQGTL | |
| | | | | VTVSSGGGGSGGGGSGGGGSGGGGSDVVM | |
| | | | | TQSPSSVSASVGDRVTITCQASQNIRTYLSWY | |
| | | | | QQKPGKAPKLLIYAAANLASGVPSRFSGSGSG | |
| | | | | TDFTLTISDLEPGDAATYYCQSTYLGTDYVGG | |
| | | | | AFGGGTKVEIK | |
| CD3 | Emiroda- | 150 | | QVTLKESGPTLVKPTETLTLTCTLSGFSLNNAR | |
| | tamab- | | | MGVSWIRQPPGKCLEWLAHIFSNDEKSYSTSL | |
| | bispecific | | | KNRLTISKDSSKTQVVLTMTNVDPVDTATYYC | |
| | anti-FLT3 | | | ARIVGYGSGWYGFFDYWGQGTLVTVSSGGG | |
| | and | | | GSGGGGSGGGGSDIQMTQSPSSLSASVGDR | |
| | anti-CD3 | | | VTITCRASQGIRNDLGWYQQKPGKAPKRLIYA | |
| | | | | ASTLQSGVPSRFSGSGSGTEFTLTISSLQPED | |
| | | | | FATYYCLQHNSYPLTFGCGTKVEIKSGGGGSE | |
| | | | | VQLVESGGGLVQPGGSLKLSCAASGFTFNKY | |
| | | | | AMNWVRQAPGKGLEWVARIRSKYNNYATYYA | |
| | | | | DSVKDRFTISRDDSKNTAYLQMNNLKTEDTAV | |
| | | | | YYCVRHGNFGNSYISYWAYWGQGTLVTVSSG | |
| | | | | GGGSGGGGSGGGGSQTVVTQEPSLTVSPGG | |
| | | | | TVTLTCGSSTGAVTSGNYPNWVQQKPGQAP | |
| | | | | RGLIGGTKFLAPGTPARFSGSLLGGKAALTL | |
| | | | | SGVQPEDEAEYYCVLWYSNRWVFGGGTKLT | |
| | | | | VLGGGGDKTHTCPPCPAPELLGGPSVFLFPP | |
| | | | | KPKDTLMISRTPEVTCVVVDVSHEDPEVKFNW | |
| | | | | YVDGVEVHNAKTKPCEEQYGSTYRCVSVLTV | |
| | | | | LHQDWLNGKEYKCKVSNKALPAPIEKTISKAK | |
| | | | | GQPREPQVYTLPPSREEMTKNQVSLTCLVKG | |
| | | | | FYPSDIAVEWESNGQPENNYKTTPPVLDSDG | |
| | | | | SFFLYSKLTVDKSRWQQGNVFSCSVMHEALH | |
| | | | | NHYTQKSLSLSPGKGGGGSGGGGSGGGGSG | |
| | | | | GGGSGGGGSGGGGSDKTHTCPPCPAPELLG | |
| | | | | GPSVFLFPPKPKDTLMISRTPEVTCVVVDVSH | |
| | | | | EDPEVKFNWYVDGVEVHNAKTKPCEEQYGST | |
| | | | | YRCVSVLTVLHQDWLNGKEYKCKVSNKALPA | |
| | | | | PIEKTISKAKGQPREPQVYTLPPSREEMTKNQ | |
| | | | | VSLTCLVKGFYPSDIAVEWESNGQPENNYKTT | |
| | | | | PPVLDSDGSFFLYSKLTVDKSRWQQGNVFSC | |
| | | | | SVMHEALHNHYTQKSLSLSPGK | |
| CD3 | Epcori- | 151 | 152 | EVQLVESGGGLVQPDRSLRLSCAASGFTFHD | EIVLTQSPATLSLSPG |
| | tamab- | | | YAMHWVRQAPGKGLEWVSTISWNSGTIGYAD | ERATLSCRASQSVSSY |
| | bispecific | | | SVKGRFTISRDNAKNSLYLQMNSLRAEDTALY | LAWYQQKPGQAPRLLI |
| | anti-CD3 | | | YCAKDIQYGNYYYGMDVWGQGTTVTVSSAST | YDASNRATGIPARFSG |
| | and anti- | | | KGPSVFPLAPSSKSTSGGTAALGCLVKDYFPE | SGSGTDFTLTISSLEP |
| | CD20 | | | PVTVSWNSGALTSGVHTFPAVLQSSGLYSLSS | EDFAVYYCQQRSNWPI |
| | antibody | | | VVTVPSSSLGTQTYICNVNHKPSNTKVDKRVE | TFGQGTRLEIKRTVAA |
| | | | | PKSCDKTHTCPPCPAPEFEGGPSVFLFPPKPK | PSVFIFPPSDEQLKSG |
| | | | | DTLMISRTPEVTCVVVAVSHEDPEVKFNWYVD | TASVVCLLNNFYPREA |
| | | | | GVEVHNAKTKPREEQYNSTYRVVSVLTVLHQ | KVQWKVDNALQSGNSQ |
| | | | | DWLNGKEYKCKVSNKALPAPIEKTISKAKGQP | ESVTEQDSKDSTYSLS |
| | | | | REPQVYTLPPSREEMTKNQVSLTCLVKGFYPS | STLTLSKADYEKHKVY |
| | | | | DIAVEWESNGQPENNYKTTPPVLDSDGSFFLY | ACEVTHQGLSSPVTKS |
| | | | | SRLTVDKSRWQQGNVFSCSVMHEALHNHYT | FNRGEC |
| | | | | QKSLSLSPG | |
| CD3E | Etevri- | 153 | | QVQLVESGGGVVQSGRSLRLSCAASGFTFRN | |
| | tamab- | | | YGMHWVRQAPGKCLEWVAVIWYDGSDKYYA | |
| | bispecific | | | DSVRGRFTISRDNSKNTLYLQMNSLRAEDTAV | |
| | anti- | | | YYCARDGYDILTGNPRDFDYWGQGTLVTVSS | |
| | EGFR and | | | GGGGSGGGGSGGGGSDTVMTQTPLSSHVTL | |
| | anti-CD3 | | | GQPASISCRSSQSLVHSDGNTYLSWLQQRPG | |
| | | | | QPPRLLIYRISRRFSGVPDRFSGSGAGTDFTL | |
| | | | | EISRVEAEDVGVYYCMQSTHVPRTFGCGTKV | |
| | | | | EIKSGGGGSEVQLVESGGGLVQPGGSLKLSC | |
| | | | | AASGFTFNKYAMNWVRQAPGKGLEWVARIRS | |
| | | | | KYNNYATYYADSVKDRFTISRDDSKNTAYLQM | |
| | | | | NNLKTEDTAVYYCVRHGNFGNSYISYWAYWG | |
| | | | | QGTLVTVSSGGGGSGGGGSGGGGSQTVVTQ | |
| | | | | EPSLTVSPGGTVTLTCGSSTGAVTSGNYPNW | |
| | | | | VQQKPGQAPRGLIGGTKFLAPGTPARFSGSL | |
| | | | | LGGKAALTLSGVQPEDEAEYYCVLWYSNRW | |
| | | | | VFGGGTKLTVLHHHHHH | |
| CD3 | F2B-CD3 | 154 | 155 | EVQLVESGGGLVQPGRSLRLSCAASGFTFDD | EIVMTQSPATLSVSPG |
| | binding | | | YAMHWVRQAPGKGLEWVSGISWNSGSIGYA | ERATLSCRASQSVSSN |
| | fragment | | | DSVKGRFTISRDNAKNSLYLQMNSLRAEDTAL | LAWYQQKPGQAPRLLI |
| | | | | YYCAKDSRGYGDYRLGGAYWGQGTLVTVSS | YGASTRATGIPARFSG |
| | | | | | SGSGTEFTLTISSLQS |
| | | | | | EDFAVYYCQQYNNWPW |
| | | | | | TFGQGTKVEIK |
| CD3E | Flotetu- | 156 | 157 | DFVMTQSPDSLAVSLGERVTMSCKSSQSLLN | QAVVTQEPSLTVSPGG |
| | zumab- | | | SGNQKNYLTWYQQKPGQPPKLLIYWASTRE | TVTLTCRSSTGAVTTS |
| | bispecific | | | SGVPDRFSGSGSGTDFTLTISSLQAEDVAVYY | NYANWVQQKPGQAPRG |
| | anti-IL3RA | | | CQNDYSYPYTFGQGTKLEIKGGGSGGGGEV | LIGGTNKRAPWTPARF |
| | and | | | QLVESGGGLVQPGGSLRLSCAASGFTFSTYA | SGSLLGGKAALTITGA |
| | anti-CD3 | | | MNWVRQAPGKGLEWVGRIRSKYNNYATYYA | QAEDEADYYCALWYSN |
| | | | | DSVKDRFTISRDDSKNSLYLQMNSLKTEDTAV | LWVFGGGTKLTVLGGG |
| | | | | YYCVRHGNFGNSYVSWFAYWGQGTLVTVSS | GSGGGGEVQLVQSGAE |
| | | | | GGCGGGKVAALKEKVAALKEKVAALKEKVAAL | LKKPGASVKVSCKASG |
| | | | | KE | YTFTDYYMKWVRQAPG |
| | | | | | QGLEWIGDIIPSNGAT |
| | | | | | FYNQKFKGRVTITVDK |
| | | | | | STSTAYMELSSLRSED |
| | | | | | TAVYYCARSHLLRASW |
| | | | | | FAYWGQGTLVTVSSGG |
| | | | | | CGGGEVAALEKEVAAL |
| | | | | | EKEVAALEKEVAALEK |
| CD3E | foralumab | 158 | 159 | QVQLVESGGGVVQPGRSLRLSCAASGFKFSG | EIVLTQSPATLSLSPG |
| | | | | YGMHWVRQAPGKGLEWVAVIWYDGSKKYYV | ERATLSCRASQSVSSY |
| | | | | DSVKGRFTISRDNSKNTLYLQMNSLRAEDTAV | LAWYQQKPGQAPRLLI |
| | | | | YYCARQMGYWHFDLWGRGTLVTVSSASTKG | YDASNRATGIPARFSG |
| | | | | PSVFPLAPSSKSTSGGTAALGCLVKDYFPEPV | SGSGTDFTLTISSLEP |
| | | | | TVSWNSGALTSGVHTFPAVLQSSGLYSLSSVV | EDFAVYYCQQRSNWPP |
| | | | | TVPSSSLGTQTYICNVNHKPSNTKVDKRVEPK | LTFGGGTKVEIKRTVA |
| | | | | SCDKTHTCPPCPAPEAEGGPSVFLFPPKPKDT | APSVFIFPPSDEQLKS |
| | | | | LMISRTPEVTCVVVDVSHEDPEVKFNWYVDG | GTASVVCLLNNFYPRE |
| | | | | VEVHNAKTKPREEQYNSTYRVVSVLTVLHQD | AKVQWKVDNALQSGNS |
| | | | | WLNGKEYKCKVSNKALPAPIEKTISKAKGQPR | QESVTEQDSKDSTYSL |
| | | | | EPQVYTLPPSREEMTKNQVSLTCLVKGFYPSD | SSTLTLSKADYEKHKV |
| | | | | IAVEWESNGQPENNYKTTPPVLDSDGSFFLYS | YACEVTHQGLSSPVTK |
| | | | | KLTVDKSRWQQGNVFSCSVMHEALHNHYTQ | SFNRGEC |
| | | | | KSLSLSPGK | |
| CD3E | Glofi- | 160 | 161 | QVQLVQSGAEVKKPGSSVKVSCKASGYAFSY | EVQLLESGGGLVQPGG |
| | tamab- | | | SWINWVRQAPGQGLEWMGRIFPGDGDTDYN | SLRLSCAASGFTFSTY |
| | bispecific | | | GKFKGRVTITADKSTSTAYMELSSLRSEDTAV | AMNWVRQAPGKGLEWV |
| | anti-CD3 | | | YYCARNVFDGYWLVYWGQGTLVTVSSASTK | SRIRSKYNNYATYYAD |
| | and | | | GPSVFPLAPSSKSTSGGTAALGCLVEDYFPEP | SVKGRFTISRDDSKNT |
| | anti-CD20 | | | VTVSWNSGALTSGVHTFPAVLQSSGLYSLSSV | LYLQMNSLRAEDTAVY |
| | antibody | | | VTVPSSSLGTQTYICNVNHKPSNTKVDEKVEP | YCVRHGNFGNSYVSWF |
| | | | | KSCDGGGGSGGGGSQAVVTQEPSLTVSPGG | AYWGQGTLVTVSSASV |
| | | | | TVTLTCGSSTGAVTTSNYANWVQEKPGQAFR | AAPSVFIFPPSDEQLK |
| | | | | GLIGGTNKRAPGTPARFSGSLLGGKAALTLSG | SGTASVVCLLNNFYPR |
| | | | | AQPEDEAEYYCALWYSNLWVFGGGTKLTVLS | EAKVQWKVDNALQSGN |
| | | | | SASTKGPSVFPLAPSSKSTSGGTAALGCLVKD | SQESVTEQDSKDSTYS |
| | | | | YFPEPVTVSWNSGALTSGVHTFPAVLQSSGL | LSSTLTLSKADYEKHK |
| | | | | YSLSSVVTVPSSSLGTQTYICNVNHKPSNTKV | VYACEVTHQGLSSPVT |
| | | | | DKKVEPKSCDKTHTCPPCPAPEAAGGPSVFL | KSFNRGEC |
| | | | | FPPKPKDTLMISRTPEVTCVVVDVSHEDPEVK | |
| | | | | FNWYVDGVEVHNAKTKPREEQYNSTYRVVSV | |
| | | | | LTVLHQDWLNGKEYKCKVSNKALGAPIEKTIS | |
| | | | | KAKGQPREPQVYTLPPCRDELTKNQVSLWCL | |
| | | | | VKGFYPSDIAVEWESNGQPENNYKTTPPVLD | |
| | | | | SDGSFFLYSKLTVDKSRWQQGNVFSCSVMHE | |
| | | | | ALHNHYTQKSLSLSPGK | |
| CD3E | Gresoni- | 162 | | QVQLVQSGAEVKKPGASVKVSCKASGYTFTG | |
| | tamab- | | | YYMHWVRQAPGQCLEWMGWINPNSGGTKY | |
| | bispecific | | | AQKFQGRVTMTRDTSISTAYMELSRLRSDDTA | |
| | anti-CD3E | | | VYYCARDRITVAGTYYYYGMDVWGQGTTVTV | |
| | and anti- | | | SSGGGGSGGGGSGGGGSDIQMTQSPSSVSA | |
| | CLDN18 | | | SVGDRVTITCRASQGVNNWLAWYQQKPGKA | |
| | | | | PKLLIYTASSLQSGVPSRFSGSGSGTDFTLTIR | |
| | | | | SLQPEDFATYYCQQANSFPITFGCGTRLEIKS | |
| | | | | GGGGSEVQLVESGGGLVQPGGSLKLSCAAS | |
| | | | | GFTFNKYAMNWVRQAPGKGLEWVARIRSKYN | |
| | | | | NYATYYADSVKDRFTISRDDSKNTAYLQMNNL | |
| | | | | KTEDTAVYYCVRHGNFGNSYISYWAYWGQGT | |
| | | | | LVTVSSGGGGSGGGGSGGGGSQTVVTQEPS | |
| | | | | LTVSPGGTVTLTCGSSTGAVTSGNYPNWVQQ | |
| | | | | KPGQAPRGLIGGTKFLAPGTPARFSGSLLGG | |
| | | | | KAALTLSGVQPEDEAEYYCVLWYSNRWVFG | |
| | | | | GGTKLTVLGGGGDKTHTCPPCPAPELLGGPS | |
| | | | | VFLFPPKPKDTLMISRTPEVTCVVVDVSHEDP | |
| | | | | EVKFNWYVDGVEVHNAKTKPCEEQYGSTYRC | |
| | | | | VSVLTVLHQDWLNGKEYKCKVSNKALPAPIEK | |
| | | | | TISKAKGQPREPQVYTLPPSREEMTKNQVSLT | |
| | | | | CLVKGFYPSDIAVEWESNGQPENNYKTTPPVL | |
| | | | | DSDGSFFLYSKLTVDKSRWQQGNVFSCSVMH | |
| | | | | EALHNHYTQKSLSLSPGKGGGGSGGGGSGG | |
| | | | | GGSGGGGSGGGGSGGGGSDKTHTCPPCPA | |
| | | | | PELLGGPSVFLFPPKPKDTLMISRTPEVTCVVV | |
| | | | | DVSHEDPEVKFNWYVDGVEVHNAKTKPCEEQ | |
| | | | | YGSTYRCVSVLTVLHQDWLNGKEYKCKVSNK | |
| | | | | ALPAPIEKTISKAKGQPREPQVYTLPPSREEMT | |
| | | | | KNQVSLTCLVKGFYPSDIAVEWESNGQPENN | |
| | | | | YKTTPPVLDSDGSFFLYSKLTVDKSRWQQGN | |
| | | | | VFSCSVMHEALHNHYTQKSLSLSPGK | |
| CD3 | hSP34.v52- | 163 | 164 | EVQLVESGGGLVQPGGSLRLSCAASGFTFNT | EAVVTQEPSLTVSPGG |
| | CD3 | | | YAMNWVRQAPGKGLEWVGRIRSKYNNYATY | TVTLTCRSSTGAVTTS |
| | binding | | | YADSVKDRFTISRDNSKNTLYLQMNSLRAEDT | NYANWFQQKPGQAPRT |
| | fragment | | | AVYYCVRHGNFGNSYVSWFAYWGQGTLVTV | LIYGTNKRAPWTPARF |
| | | | | SS | SGSLLGGKAALTLSGA |
| | | | | | QPEDEAEYYCALWYSN |
| | | | | | LWVFGGGTKLTVL |
| CD3 | hu38E4v1- | 165 | 166 | EVQLVQSGAEVKKPGASVKVSCKASGFTFTS | DIVMTQSPDSLAVSLG |
| | CD3 | | | YYIHWVRQAPGQGLEWIGWIYPENDNTKYNE | ERATINCKSSQSLLNS |
| | binding | | | KFKDRVTITADTSTSTAYLELSSLRSEDTAVYY | RTRKNYLAWYQQKPGQ |
| | fragment | | | CARDGYSRYYFDYWGQGTLVTVSS | SPKLLIYWTSTRKSGV |
| | | | | | PDRFSGSGSGTDFTLT |
| | | | | | ISSLQAEDVAVYYCKQ |
| | | | | | SFILRTFGQGTKVEIK |
| CD3 | IAB25M- | 167 | 168 | EVQLVESGGGLVQPGGSLRLSCAASGFNIKDT | DVQITQSPSSLSASVG |
| | CD3 | | | YIHFVRQAPGKGLEWIGRIDPANDNTLYASKF | DRVTITCRTSRSISQY |
| | binding | | | QGKATISADTSKNTAYLQMNSLRAEDTAVYYC | LAWYQQKPGKVPKLLI |
| | fragment | | | GRGYGYYVFDHWGQGTLVTVSS | YSGSTLQSGVPSRFSG |
| | | | | | SGSGTDFTLTISSLQP |
| | | | | | EDVATYYCQQHNENPL |
| | | | | | TFGGGTKVEIK |
| CD3E | Imvotamab- | 169 | | QVQLVQSGAEVKKPGASVKVSCKASGYTFISY | |
| | bispecific | | | TMHWVRQAPGQGLEWMGYINPRSGYTHYNQ | |
| | anti-CD3 | | | KLKDKATLTADKSASTAYMELSSLRSEDTAVY | |
| | and | | | YCARSAYYDYDGFAYWGQGTLVTVSSGGGG | |
| | anti-CD20 | | | SGGGGSGGGGSDIQMTQSPSSLSASVGDRV | |
| | antibody | | | TITCSASSSVSYMNWYQQKPGKAPKRLIYDT | |
| | | | | SKLASGVPSRFSGSGSGTDFTLTISSLQPEDF | |
| | | | | ATYYCQQWSSNPPTFGGGTKVEIKGGGGSG | |
| | | | | GGGSGGGGSQEDERIVLVDNKCKCARITSRII | |
| | | | | RSSEDPNEDIVERNIRIIVPLNNRENISDPTSPL | |
| | | | | RTRFVYHLSDLCKKCDPTEVELDNQIVTATQS | |
| | | | | NICDEDSATETCYTYDRNKCYTAVVPLVYGGE | |
| | | | | TKMVETALTPDACYPDGGGGSGGGGSGGGG | |
| | | | | SDAHKSEVAHRFKDLGEENFKALVLIAFAQYL | |
| | | | | QQCPFEDHVKLVNEVTEFAKTCVADESAENC | |
| | | | | DKSLHTLFGDKLCTVATLRETYGEMADCCAK | |
| | | | | QEPERNECFLQHKDDNPNLPRLVRPEVDVMC | |
| | | | | TAFHDNEETFLKKYLYEIARRHPYFYAPELLFF | |
| | | | | AKRYKAAFTECCQAADKAACLLPKLDELRDEG | |
| | | | | KASSAKQRLKCASLQKFGERAFKAWAVARLS | |
| | | | | QRFPKAEFAEVSKLVTDLTKVHTECCHGDLLE | |
| | | | | CADDRADLAKYICENQDSISSKLKECCEKPLLE | |
| | | | | KSHCIAEVENDEMPADLPSLAADFVESKDVCK | |
| | | | | NYAEAKDVFLGMFLYEYARRHPDYSVVLLLRL | |
| | | | | AKTYETTLEKCCAAADPHECYAKVFDEFKPLV | |
| | | | | EEPQNLIKQNCELFKQLGEYKFQNALLVRYTK | |
| | | | | KVPQVSTPTLVEVSRNLGKVGSKCCKHPEAK | |
| | | | | RMPCAEDYLSVVLNQLCVLHEKTPVSDRVTK | |
| | | | | CCTESLVNRRPCFSALEVDETYVPKEFNAETF | |
| | | | | TFHADICTLSEKERQIKKQTALVELVKHKPKAT | |
| | | | | KEQLKAVMDDFAAFVEKCCKADDKETCFAEE | |
| | | | | GKKLVAASQAALGL | |
| CD3 | L2K-CD3 | 170 | 171 | DIKLQSGAELARPGASVKMSCKTSGYTFTRYT | DIQLTQSPAIMSASPG |
| | binding | | | MHWVKQRPGQGLEWIGYINPSRGYTNYNQKF | EKVTMTCRASSSVSYM |
| | fragment | | | KDKATLTTDKSSSTAYMQLSSLTSEDSAVYYC | NWYQQKSGTSPKRWIY |
| | | | | ARYYDDHYCLDYWGQGTTLTVSS | DTSKVASGVPYRFSGS |
| | | | | | GSGTSYSLTISSMEAE |
| | | | | | DAATYYCQQWSSNPLT |
| | | | | | FGAGTKLELK |
| CD3E | Linvosel- | 172 | | EVQLVESGGGLVQPGRSLRLSCAASGFTFDD | |
| | tamab- | | | YSMHWVRQAPGKGLEWVSGISWNSGSKGYA | |
| | bispecific | | | DSVKGRFTISRDNAKNSLYLQMNSLRAEDTAL | |
| | anti-CD3E | | | YYCAKYGSGYGKFYHYGLDVWGQGTTVTVS | |
| | and anti- | | | SASTKGPSVFPLAPCSRSTSESTAALGCLVKD | |
| | BCMA | | | YFPEPVTVSWNSGALTSGVHTFPAVLQSSGL | |
| | antibody | | | YSLSSVVTVPSSSLGTKTYTCNVDHKPSNTKV | |
| | | | | DKRVESKYGPPCPPCPAPPVAGPSVFLFPPK | |
| | | | | PKDTLMISRTPEVTCVVVDVSQEDPEVQFNW | |
| | | | | YVDGVEVHNAKTKPREEQFNSTYRVVSVLTVL | |
| | | | | HQDWLNGKEYKCKVSNKGLPSSIEKTISKAKG | |
| | | | | QPREPQVYTLPPSQEEMTKNQVSLTCLVKGF | |
| | | | | YPSDIAVEWESNGQPENNYKTTPPVLDSDGS | |
| | | | | FFLYSRLTVDKSRWQEGNVFSCSVMHEALHN | |
| | | | | RFTQKSLSLSPGK | |
| CD3 | Mosunetu- | 173 | 174 | EVQLVQSGAEVKKPGASVKVSCKASGYTFTN | DIVMTQSPDSLAVSLG |
| | zumab- | | | YYIHWVRQAPGQGLEWIGWIYPGDGNTKYNE | ERATINCKSSQSLLNS |
| | bispecific | | | KFKGRATLTADTSTSTAYLELSSLRSEDTAVYY | RTRKNYLAWYQQKPGQ |
| | anti-CD3 | | | CARDSYSNYYFDYWGQGTLVTVSSASTKGPS | PPKLLIYWASTRESGV |
| | and | | | VFPLAPSSKSTSGGTAALGCLVKDYFPEPVTV | PDRFSGSGSGTDFTLT |
| | anti-CD20 | | | SWNSGALTSGVHTFPAVLQSSGLYSLSSVVTV | ISSLQAEDVAVYYCTQ |
| | antibody | | | PSSSLGTQTYICNVNHKPSNTKVDKKVEPKSC | SFILRTFGQGTKVEIK |
| | | | | DKTHTCPPCPAPELLGGPSVFLFPPKPKDTLM | RTVAAPSVFIFPPSDE |
| | | | | ISRTPEVTCVVVDVSHEDPEVKFNWYVDGVE | QLKSGTASVVCLLNNF |
| | | | | VHNAKTKPREEQYGSTYRVVSVLTVLHQDWL | YPREAKVQWKVDNALQ |
| | | | | NGKEYKCKVSNKALPAPIEKTISKAKGQPREP | SGNSQESVTEQDSKDS |
| | | | | QVYTLPPSREEMTKNQVSLSCAVKGFYPSDIA | TYSLSSTLTLSKADYE |
| | | | | VEWESNGQPENNYKTTPPVLDSDGSFFLVSK | KHKVYACEVTHQGLSS |
| | | | | LTVDKSRWQQGNVFSCSVMHEALHNHYTQK | PVTKSFNRGEC |
| | | | | SLSLSPGK | |
| CD3E | muromonab | 175 | 176 | QVQLQQSGAELARPGASVKMSCKASGYTFTR | QIVLTQSPAIMSASPG |
| | | | | YTMHWVKQRPGQGLEWIGYINPSRGYTNYNQ | EKVTMTCSASSSVSYM |
| | | | | KFKDKATLTTDKSSSTAYMQLSSLTSEDSAVY | NWYQQKSGTSPKRWIY |
| | | | | YCARYYDDHYCLDYWGQGTTLTVSSAKTTAP | DTSKLASGVPAHFRGS |
| | | | | SVYPLAPVCGGTTGSSVTLGCLVKGYFPEPVT | GSGTSYSLTISGMEAE |
| | | | | LTWNSGSLSSGVHTFPAVLQSDLYTLSSSVTV | DAATYYCQQWSSNPFT |
| | | | | TSSTWPSQSITCNVAHPASSTKVDKKIEPRPK | FGSGTKLEINRADTAP |
| | | | | SCDKTHTCPPCPAPELLGGPSVFLFPPKPKDT | TVSIFPPSSEQLTSGG |
| | | | | LMISRTPEVTCVVVDVSHEDPEVKFNWYVDG | ASVVCFLNNFYPKDIN |
| | | | | VEVHNAKTKPREEQYNSTYRVVSVLTVLHQD | VKWKIDGSERQNGVLN |
| | | | | WLNGKEYKCKVSNKALPAPIEKTISKAKGQPR | SWTDQDSKDSTYSMSS |
| | | | | EPQVYTLPPSRDELTKNQVSLTCLVKGFYPSD | TLTLTKDEYERHNSYT |
| | | | | IAVEWESNGQPENNYKTTPPVLDSDGSFFLYS | CEATHKTSTSPIVKSF |
| | | | | KLTVDKSRWQQGNVFSCSVMHEALHNHYTQ | NRNEC |
| | | | | KSLSLSPGK | |
| CD3 | nivatro- | | 177 | | EIVMTQTPATLSVSAG |
| | tamab- | | | | ERVTITCKASQSVSND |
| | bispecific | | | | VTWYQQKPGQAPRLLI |
| | anti-CD3E | | | | YSASNRYSGVPARFSG |
| | and | | | | SGYGTEFTFTISSVQS |
| | anti-GD2 | | | | EDFAVYFCQQDYSSFG |
| | antibody | | | | QGTKLEIKRTVAAPSV |
| | (anti-CD3E | | | | FIFPPSDEQLKSGTAS |
| | binding | | | | VVCLLNNFYPREAKVQ |
| | regions | | | | WKVDNALQSGNSQESV |
| | are on | | | | TEQDSKDSTYSLSSTL |
| | light | | | | TLSKADYEKHKVYACE |
| | chain | | | | VTHQGLSSPVTKSFNR |
| | only) | | | | GECTSGGGGSGGGGSG |
| | | | | | GGGSQVQLVQSGGGVV |
| | | | | | QPGRSLRLSCKASGYT |
| | | | | | FTRYTMHWVRQAPGKC |
| | | | | | LEWIGYINPSRGYTNY |
| | | | | | NQKFKDRFTISRDNSK |
| | | | | | NTAFLQMDSLRPEDTG |
| | | | | | VYFCARYYDDHYSLDY |
| | | | | | WGQGTPVTVSSGGGGS |
| | | | | | GGGGSGGGGSGGGGSG |
| | | | | | GGGSGGGGSDIQMTQS |
| | | | | | PSSLSASVGDRVTITC |
| | | | | | SASSSVSYMNWYQQTP |
| | | | | | GKAPKRWIYDTSKLAS |
| | | | | | GVPSRFSGSGSGTDYT |
| | | | | | FTISSLQPEDIATYYC |
| | | | | | QQWSSNPFTFGCGTKL |
| | | | | | QITR |
| CD3 | obrinda- | 178 | 179 | DIQLTQSPSFLSASVGDRVTITCKASQNVDTN | QAVVTQEPSLTVSPGG |
| | tamab | | | VAWYQQKPGKAPKALIYSASYRYSGVPSRFS | TVTLTCRSSTGAVTTS |
| | | | | GSGSGTDFTLTISSLQPEDFATYYCQQYNNYP | NYANWVQQKPGQAPRG |
| | | | | FTFGQGTKLEIKGGGSGGGGEVQLVESGGGL | LIGGTNKRAPWTPARF |
| | | | | VQPGGSLRLSCAASGFTFSTYAMNWVRQAP | SGSLLGGKAALTITGA |
| | | | | GKGLEWVGRIRSKYNNYATYYADSVKDRFTIS | QAEDEADYYCALWYSN |
| | | | | RDDSKNSLYLQMNSLKTEDTAVYYCVRHGNF | LWVFGGGTKLTVLGGG |
| | | | | GNSYVSWFAYWGQGTLVTVSSGGGGGEVA | GSGGGGEVQLVESGGG |
| | | | | ALEKEVAALEKEVAALEKEVAALEKGGGDKTH | LVQPGGSLRLSCAASG |
| | | | | TCPPCPAPEAAGGPSVFLFPPKPKDTLMISRT | FTFSSFGMHWVRQAPG |
| | | | | PEVTCVVVDVSHEDPEVKFNWYVDGVEVHNA | KGLEWVAYISSDSSAI |
| | | | | KTKPREEQYNSTYRVVSVLTVLHQDWLNGKE | YYADTVKGRFTISRDN |
| | | | | YKCKVSNKALPAPIEKTISKAKGQPREPQVYTL | AKNSLYLQMNSLRDED |
| | | | | PPSREEMTKNQVSLWCLVKGFYPSDIAVEWE | TAVYYCGRGRENIYYG |
| | | | | SNGQPENNYKTTPPVLDSDGSFFLYSKLTVDK | SRLDYWGQGTTVTVSS |
| | | | | SRWQQGNVFSCSVMHEALHNHYTQKSLSLSP | GGCGGGKVAALKEKVA |
| | | | | Gk | ALKEKVAALKEKVAAL |
| | | | | | KE |
| CD3E | Odronex- | 180 | | EVQLVESGGGLVQPGRSLRLSCAASGFTFDD | |
| | tamab- | | | YTMHWVRQAPGKGLEWVSGISWNSGSIGYA | |
| | bispecific | | | DSVKGRFTISRDNAKKSLYLQMNSLRAEDTAL | |
| | anti-CD20 | | | YYCAKDNSGYGHYYYGMDVWGQGTTVTVAS | |
| | anti-CD3 | | | ASTKGPSVFPLAPCSRSTSESTAALGCLVKDY | |
| | antibody | | | FPEPVTVSWNSGALTSGVHTFPAVLQSSGLY | |
| | | | | SLSSVVTVPSSSLGTKTYTCNVDHKPSNTKVD | |
| | | | | KRVESKYGPPCPPCPAPPVAGPSVFLFPPKPK | |
| | | | | DTLMISRTPEVTCVVVDVSQEDPEVQFNWYV | |
| | | | | DGVEVHNAKTKPREEQFNSTYRVVSVLTVLH | |
| | | | | QDWLNGKEYKCKVSNKGLPSSIEKTISKAKGQ | |
| | | | | PREPQVYTLPPSQEEMTKNQVSLTCLVKGFY | |
| | | | | PSDIAVEWESNGQPENNYKTTPPVLDSDGSF | |
| | | | | FLYSRLTVDKSRWQEGNVFSCSVMHEALHNR | |
| | | | | FTQKSLSLSLGK | |
| CD3E | otelixi- | 181 | 182 | EVQLLESGGGLVQPGGSLRLSCAASGFTFSS | DIQLTQPNSVSTSLGS |
| | zumab | | | FPMAWVRQAPGKGLEWVSTISTSGGRTYYRD | TVKLSCTLSSGNIENN |
| | | | | SVKGRFTISRDNSKNTLYLQMNSLRAEDTAVY | YVHWYQLYEGRSPTTM |
| | | | | YCAKFRQYSGGFDYWGQGTLVTVSSASTKGP | IYDDDKRPDGVPDRFS |
| | | | | SVFPLAPSSKSTSGGTAALGCLVKDYFPEPVT | GSIDRSSNSAFLTIHN |
| | | | | VSWNSGALTSGVHTFPAVLQSSGLYSLSSVVT | VAIEDEAIYFCHSYVS |
| | | | | VPSSSLGTQTYICNVNHKPSNTKVDKKVEPKS | SFNVFGGGTKLTVLRQ |
| | | | | CDKTHTCPPCPAPELLGGPSVFLFPPKPKDTL | PKAAPSVTLFPPSSEE |
| | | | | MISRTPEVTCVVVDVSHEDPEVKFNWYVDGV | LQANKATLVCLISDFY |
| | | | | EVHNAKTKPREEQYASTYRVVSVLTVLHQDW | PGAVTVAWKADSSPVK |
| | | | | LNGKEYKCKVSNKALPAPIEKTISKAKGQPREP | AGVETTTPSKQSNNKY |
| | | | | QVYTLPPSRDELTKNQVSLTCLVKGFYPSDIA | AASSYLSLTPEQWKSH |
| | | | | VEWESNGQPENNYKTTPPVLDSDGSFFLYSK | RSYSCQVTHEGSTVEK |
| | | | | LTVDKSRWQQGNVFSCSVMHEALHNHYTQK | TVAPTECS |
| | | | | SLSLSPGK | |
| CD3 | Pacanalo- | 183 | | QVQLVQSGAEVKKPGASVKVSCKASGYTFTN | |
| | tabab- | | | HIIHWVRQAPGQGLEWMGYINPYPGYHAYNE | |
| | BiTE with | | | KFQGRATMTSDTSTSTVYMELSSLRSEDTAVY | |
| | affinity | | | YCARDGYYRDTDVLDYWGQGTLVTVSSGGG | |
| | against | | | GSGGGGSGGGGSDIQMTQSPSSLSASVGDR | |
| | BCMA and | | | VTITCQASQDISNYLNWYQQKPGKAPKLLIYYT | |
| | CD3E | | | SRLHTGVPSRFSGSGSGTDFTFTISSLEPEDIA | |
| | | | | TYYCQQGNTLPWTFGQGTKVEIKSGGGGSEV | |
| | | | | QLVESGGGLVQPGGSLKLSCAASGFTFNKYA | |
| | | | | MNWVRQAPGKGLEWVARIRSKYNNYATYYA | |
| | | | | DSVKDRFTISRDDSKNTAYLQMNNLKTEDTAV | |
| | | | | YYCVRHGNFGNSYISYWAYWGQGTLVTVSSG | |
| | | | | GGGSGGGGSGGGGSQTVVTQEPSLTVSPGG | |
| | | | | TVTLTCGSSTGAVTSGNYPNWVQQKPGQAP | |
| | | | | RGLIGGTKFLAPGTPARFSGSLLGGKAALTL | |
| | | | | SGVQPEDEAEYYCVLWYSNRWVFGGGTKLT | |
| | | | | VLHHHHHH | |
| CD3E | Pasotuxi- | 184 | | QVQLVESGGGLVKPGESLRLSCAASGFTFSD | |
| | zumab- | | | YYMYWVRQAPGKGLEWVAIISDGGYYTYYSDI | |
| | bispecific | | | IKGRFTISRDNAKNSLYLQMNSLKAEDTAVYY | |
| | anti-CD3E | | | CARGFPLLRHGAMDYWGQGTLVTVSSGGGG | |
| | and anti- | | | SGGGGSGGGGSDIQMTQSPSSLSASVGDRV | |
| | FOLH1 | | | TITCKASQNVDTNVAWYQQKPGQAPKSLIYSA | |
| | antibody | | | SYRYSDVPSRFSGSASGTDFTLTISSVQSEDF | |
| | | | | ATYYCQQYDSYPYTFGGGTKLEIKSGGGGSE | |
| | | | | VQLVESGGGLVQPGGSLKLSCAASGFTFNKY | |
| | | | | AMNWVRQAPGKGLEWVARIRSKYNNYATYYA | |
| | | | | DSVKDRFTISRDDSKNTAYLQMNNLKTEDTAV | |
| | | | | YYCVRHGNFGNSYISYWAYWGQGTLVTVSSG | |
| | | | | GGGSGGGGSGGGGSQTVVTQEPSLTVSPGG | |
| | | | | TVTLTCGSSTGAVTSGNYPNWVQQKPGQAP | |
| | | | | RGLIGGTKFLAPGTPARFSGSLLGGKAALTL | |
| | | | | SGVQPEDEAEYYCVLWYSNRWVFGGGTKLT | |
| | | | | VLHHHHHH | |
| CD3 | Pavuru- | 185 | | QVQLVQSGAEVKKPGASVKVSCKASGYTFTN | |
| | tamab- | | | HIIHWVRQAPGQCLEWMGYINPYPGYHAYNE | |
| | BITE with | | | KFQGRATMTSDTSTSTVYMELSSLRSEDTAVY | |
| | affinity | | | YCARDGYYRDTDVLDYWGQGTLVTVSSGGG | |
| | to BCMA | | | GSGGGGGGGGSDIQMTQSPSSLSASVGDR | |
| | and CD3E | | | VTITCQASQDISNYLNWYQQKPGKAPKLLIYYT | |
| | | | | SRLHTGVPSRFSGSGSGTDFTFTISSLEPEDIA | |
| | | | | TYYCQQGNTLPWTFGCGTKVEIKSGGGGSEV | |
| | | | | QLVESGGGLVQPGGSLKLSCAASGFTENKYA | |
| | | | | MNWVRQAPGKGLEWVARIRSKYNNYATYYA | |
| | | | | DSVKDRFTISRDDSKNTAYLQMNNLKTEDTAV | |
| | | | | YYCVRHGNFGNSYISYWAYWGQGTLVTVSSG | |
| | | | | GGGSGGGGSGGGGSQTVVTQEPSLTVSPGG | |
| | | | | TVTLTCGSSTGAVTSGNYPNWVQQKPGQAP | |
| | | | | RGLIGGTKFLAPGTPARFSGSLLGGKAALTL | |
| | | | | SGVQPEDEAEYYCVLWYSNRWVFGGGTKLT | |
| | | | | VLGGGGDKTHTCPPCPAPELLGGPSVFLFPP | |
| | | | | KPKDTLMISRTPEVTCVVVDVSHEDPEVKFNW | |
| | | | | YVDGVEVHNAKTKPCEEQYGSTYRCVSVLTV | |
| | | | | LHQDWLNGKEYKCKVSNKALPAPIEKTISKAK | |
| | | | | GQPREPQVYTLPPSREEMTKNQVSLTCLVKG | |
| | | | | FYPSDIAVEWESNGQPENNYKTTPPVLDSDG | |
| | | | | SFFLYSKLTVDKSRWQQGNVFSCSVMHEALH | |
| | | | | NHYTQKSLSLSPGKGGGGSGGGGSGGGGSG | |
| | | | | GGGSGGGGSGGGGSDKTHTCPPCPAPELLG | |
| | | | | GPSVFLFPPKPKDTLMISRTPEVTCVVVDVSH | |
| | | | | EDPEVKFNWYVDGVEVHNAKTKPCEEQYGST | |
| | | | | YRCVSVLTVLHQDWLNGKEYKCKVSNKALPA | |
| | | | | PIEKTISKAKGQPREPQVYTLPPSREEMTKNQ | |
| | | | | VSLTCLVKGFYPSDIAVEWESNGQPENNYKTT | |
| | | | | PPVLDSDGSFFLYSKLTVDKSRWQQGNVFSC | |
| | | | | SVMHEALHNHYTQKSLSLSPGK | |
| CD3E | Plamo- | 186 | | EVQLVESGGGLVQPGGSLRLSCAASGFTFST | |
| | tamab- | | | YAMNWVRQAPGKGLEWVGRIRSKYNNYATY | |
| | bispecific | | | YADSVKGRFTISRDDSKNTLYLQMNSLRAEDT | |
| | anti-CD3 | | | AVYYCVRHGNFGDSYVSWFAYWGQGTLVTV | |
| | and | | | SSGKPGSGKPGSGKPGSGKPGSQAVVTQEP | |
| | anti-CD20 | | | SLTVSPGGTVTLTCGSSTGAVTTSNYANWVQ | |
| | antibody | | | QKPGKSPRGLIGGTNKRAPGVPARFSGSLLG | |
| | | | | GKAALTISGAQPEDEADYYCALWYSNHWVF | |
| | | | | GGGTKLTVLEPKSSDKTHTCPPCPAPPVAGP | |
| | | | | SVFLFPPKPKDTLMISRTPEVTCVVVDVKHED | |
| | | | | PEVKFNWYVDGVEVHNAKTKPREEQYNSTYR | |
| | | | | VVSVLTVLHQDWLNGKEYKCKVSNKALPAPIE | |
| | | | | KTISKAKGQPREPQVYTLPPSREQMTKNQVKL | |
| | | | | TCLVKGFYPSDIAVEWESNGQPENNYKTTPPV | |
| | | | | LDSDGSFFLYSKLTVDKSRWQQGNVFSCSVM | |
| | | | | HEALHNHYTQKSLSLSPGK | |
| CD3E | Runimo- | 187 | 188 | EVQLVQSGAEVKKPGASVKVSCKASGYTFTN | DIVMTQSPDSLAVSLG |
| | tamab- | | | YYIHWVRQAPGQGLEWIGWIYPGDGNTKYNE | ERATINCKSSQSLLNS |
| | bispecific | | | KFKGRATLTADTSTSTAYLELSSLRSEDTAVYY | RTRKNYLAWYQQKPGQ |
| | anti-HER2 | | | CARDSYSNYYFDYWGQGTLVTVSSASTKGPS | PPKLLIYWASTRESGV |
| | and | | | VFPLAPSSKSTSGGTAALGCLVKDYFPEPVTV | PDRFSGSGSGTDFTLT |
| | anti-CD3 | | | SWNSGALTSGVHTFPAVLQSSGLYSLSSVVTV | ISSLQAEDVAVYYCTQ |
| | | | | PSSSLGTQTYICNVNHKPSNTKVDKKVEPKSC | SFILRTFGQGTKVEIK |
| | | | | DKTHTCPPCPAPELLGGPSVFLFPPKPKDTLM | RTVAAPSVFIFPPSDE |
| | | | | ISRTPEVTCVVVDVSHEDPEVKFNWYVDGVE | QLKSGTASVVCLLNNF |
| | | | | VHNAKTKPREEQYGSTYRVVSVLTVLHQDWL | YPREAKVQWKVDNALQ |
| | | | | NGKEYKCKVSNKALPAPIEKTISKAKGQPREP | SGNSQESVTEQDSKDS |
| | | | | QVYTLPPSREEMTKNQVSLSCAVKGFYPSDIA | TYSLSSTLTLSKADYE |
| | | | | VEWESNGQPENNYKTTPPVLDSDGSFFLVSK | KHKVYACEVTHQGLSS |
| | | | | LTVDKSRWQQGNVFSCSVMHEALHNHYTQK | PVTKSFNRGEC |
| | | | | SLSLSPGK | |
| CD3 | Solitomab- | 189 | | ELVMTQSPSSLTVTAGEKVTMSCKSSQSLLNS | |
| | bispecific | | | GNQKNYLTWYQQKPGQPPKLLIYWASTRESG | |
| | anti-CD3 | | | VPDRFTGSGSGTDFTLTISSVQAEDLAVYYCQ | |
| | and anti- | | | NDYSYPLTFGAGTKLEIKGGGGSGGGGSGGG | |
| | EPCAM | | | GSEVQLLEQSGAELVRPGTSVKISCKASGYAF | |
| | antibody | | | TNYWLGWVKQRPGHGLEWIGDIFPGSGNIHY | |
| | | | | NEKFKGKATLTADKSSSTAYMQLSSLTFEDSA | |
| | | | | VYFCARLRNWDEPMDYWGQGTTVTVSSGGG | |
| | | | | GSDVQLVQSGAEVKKPGASVKVSCKASGYT | |
| | | | | FTRYTMHWVRQAPGQGLEWIGYINPSRGYTN | |
| | | | | YADSVKGRFTITTDKSTSTAYMELSSLRSEDT | |
| | | | | ATYYCARYYDDHYCLDYWGQGTTVTVSSGE | |
| | | | | GTSTGSGGSGGSGGADDIVLTQSPATLSLSP | |
| | | | | GERATLSCRASQSVSYMNWYQQKPGKAPKR | |
| | | | | WIYDTSKVASGVPARFSGSGSGTDYSLTINSL | |
| | | | | EAEDAATYYCQQWSSNPLTFGGGTKVEIKHH | |
| | | | | HHHH | |
| CD3 | SP34-CD3 | 190 | 191 | EVQLVESGGGLVQPKGSLKLSCAASGFTFNT | QAVVTQESALTTSPGE |
| | binding | | | YAMNWVRQAPGKGLEWVARIRSKYNNYATYY | TVTLTCRSSTGAVTTS |
| | fragment | | | ADSVKDRFTISRDDSQSILYLQMNNLKTEDTA | NYANWVQEKPDHLFTG |
| | | | | MYYCVRHGNFGNSYVSWFAYWGQGTLVTVS | LIGGTNKRAPGVPARF |
| | | | | A | SGSLIGDKAALTITGA |
| | | | | | QTEDEAIYFCALWYSN |
| | | | | | LWVFGGGTKLTVL |
| CD3E | Talque- | 192 | 193 | EVQLVESGGGLVQPGGSLRLSCAASGFTFNT | QTVVTQEPSLTVSPGG |
| | tamab- | | | YAMNWVRQAPGKGLEWVARIRSKYNNYATYY | TVTLTCRSSTGAVTTS |
| | | | | AASVKGRFTISRDDSKNSLYLQMNSLKTEDTA | NYANWVQQKPGQAPRG |
| | | | | VYYCARHGNFGNSYVSWFAYWGQGTLVTVS | LIGGTNKRAPGTPARF |
| | | | | SASTKGPSVFPLAPCSRSTSESTAALGCLVKD | SGSLLGGKAALTLSGV |
| | bispecific | | | YFPEPVTVSWNSGALTSGVHTFPAVLQSSGL | QPEDEAEYYCALWYSN |
| | anti- | | | YSLSSVVTVPSSSLGTKTYTCNVDHKPSNTKV | LWVFGGGTKLTVLGQP |
| | GPRC5D | | | DKRVESKYGPPCPPCPAPEAAGGPSVFLFPP | KAAPSVTLFPPSSEEL |
| | and | | | KPKDTLMISRTPEVTCVVVDVSQEDPEVQFN | QANKATLVCLISDFYP |
| | anti-CD3 | | | WYVDGVEVHNAKTKPREEQFNSTYRVVSVLT | GAVTVAWKADSSPVKA |
| | | | | VLHQDWLNGKEYKCKVSNKGLPSSIEKTISKA | GVETTTPSKQSNNKYA |
| | | | | KGQPREPQVYTLPPSQEEMTKNQVSLTCLVK | ASSYLSLTPEQWKSHR |
| | | | | GFYPSDIAVEWESNGQPENNYKTTPPVLDSD | SYSCQVTHEGSTVEKT |
| | | | | GSFLLYSKLTVDKSRWQEGNVFSCSVMHEAL | VAPTECS |
| | | | | HNHYTQKSLSLSLGK | |
| CD3E | Tarla- | 194 | | QVQLQESGPGLVKPSETLSLTCTVSGGSISSY | |
| | tamab- | | | YWSWIRQPPGKCLEWIGYVYYSGTTNYNPSL | |
| | bispecific | | | KSRVTISVDTSKNQFSLKLSSVTAADTAVYYCA | |
| | anti-CD3 | | | SIAVTGFYFDYWGQGTLVTVSSGGGGSGGG | |
| | and | | | GSGGGGSEIVLTQSPGTLSLSPGERVTLSCRA | |
| | anti-DLL3 | | | SQRVNNNYLAWYQQRPGQAPRLLIYGASSRA | |
| | | | | TGIPDRFSGSGSGTDFTLTISRLEPEDFAVYYC | |
| | | | | QQYDRSPLTFGCGTKLEIKSGGGGSEVQLVE | |
| | | | | SGGGLVQPGGSLKLSCAASGFTFNKYAMNW | |
| | | | | VRQAPGKGLEWVARIRSKYNNYATYYADSVK | |
| | | | | DRFTISRDDSKNTAYLQMNNLKTEDTAVYYCV | |
| | | | | RHGNFGNSYISYWAYWGQGTLVTVSSGGGG | |
| | | | | SGGGGSGGGGSQTVVTQEPSLTVSPGGTVT | |
| | | | | LTCGSSTGAVTSGNYPNWVQQKPGQAPRGL | |
| | | | | IGGTKFLAPGTPARFSGSLLGGKAALTLSGV | |
| | | | | QPEDEAEYYCVLWYSNRWVFGGGTKLTVLG | |
| | | | | GGGDKTHTCPPCPAPELLGGPSVFLFPPKPK | |
| | | | | DTLMISRTPEVTCVVVDVSHEDPEVKFNWYVD | |
| | | | | GVEVHNAKTKPCEEQYGSTYRCVSVLTVLHQ | |
| | | | | DWLNGKEYKCKVSNKALPAPIEKTISKAKGQP | |
| | | | | REPQVYTLPPSREEMTKNQVSLTCLVKGFYPS | |
| | | | | DIAVEWESNGQPENNYKTTPPVLDSDGSFFLY | |
| | | | | SKLTVDKSRWQQGNVFSCSVMHEALHNHYT | |
| | | | | QKSLSLSPGGGGSGGGGSGGGGSGGGGSG | |
| | | | | GGGSGGGGSDKTHTCPPCPAPELLGGPSVFL | |
| | | | | FPPKPKDTLMISRTPEVTCVVVDVSHEDPEVK | |
| | | | | FNWYVDGVEVHNAKTKPCEEQYGSTYRCVSV | |
| | | | | LTVLHQDWLNGKEYKCKVSNKALPAPIEKTISK | |
| | | | | AKGQPREPQVYTLPPSREEMTKNQVSLTCLV | |
| | | | | KGFYPSDIAVEWESNGQPENNYKTTPPVLDS | |
| | | | | DGSFFLYSKLTVDKSRWQQGNVFSCSVMHEA | |
| | | | | LHNHYTQKSLSLSPGK | |
| CD3 | Tebenta- | 195 | | AIQMTQSPSSLSASVGDRVTITCRASQDIRNY | |
| | fusp- | | | LNWYQQKPGKAPKLLIYYTSRLESGVPSRFS | |
| | anti-CD3 | | | GSGSGTDYTLTISSLQPEDFATYYCQQGNTLP | |
| | fused to | | | WTFGQGTKVEIKGGGGSGGGGSGGGGSGG | |
| | engineered | | | GGSGGGSEVQLVESGGGLVQPGGSLRLSCA | |
| | T cell | | | ASGYSFTGYTMNWVRQAPGKGLEWVALINPY | |
| | receptor | | | KGVSTYNQKFKDRFTISVDKSKNTAYLQMNSL | |
| | | | | RAEDTAVYYCARSGYYGDSDWYFDVWGQGT | |
| | | | | LVTVSSGGGGSDGGITQSPKYLFRKEGQNVT | |
| | | | | LSCEQNLNHDAMYWYRQDPGQGLRLIYYSW | |
| | | | | AQGDFQKGDIAEGYSVSREKKESFPLTVTSAQ | |
| | | | | KNPTAFYLCASSWGAPYEQYFGPGTRLTVTE | |
| | | | | DLKNVFPPEVAVFEPSEAEISHTQKATLVCLAT | |
| | | | | GFYPDHVELSWWVNGKEVHSGVCTDPQPLK | |
| | | | | EQPALNDSRYALSSRLRVSATFWQDPRNHFR | |
| | | | | CQVQFYGLSENDEWTQDRAKPVTQIVSAEAW | |
| | | | | GRAD | |
| CD3 | Teben- | 196 | 197 | EVQLVESGGGLVQPGGSLRLSCAASGYSFTG | AIQMTQSPSSLSASVG |
| | tausp- | | | YTMNWVRQAPGKGLEWVALINPYKGVSTYNQ | DRVTITCRASQDIRNY |
| | CD3 | | | KFKDRFTISVDKSKNTAYLQMNSLRAEDTAVY | LNWYQQKPGKAPKLLI |
| | binding | | | YCARSGYYGDSDWYFDVWGQGTLVTVSS | YYTSRLESGVPSRFSG |
| | fragment | | | | SGSGTDYTLTISSLQP |
| | | | | | EDFATYYCQQGNTLP |
| | | | | | WTFGQGTKVEIK |
| CD3E | Teclis- | 198 | 199 | EVQLVESGGGLVQPGGSLRLSCAASGFTFNT | QTVVTQEPSLTVSPGG |
| | tamab- | | | YAMNWVRQAPGKGLEWVARIRSKYNNYATYY | TVTLTCRSSTGAVTTS |
| | bispecific | | | AASVKGRFTISRDDSKNSLYLQMNSLKTEDTA | NYANWVQQKPGQAPRG |
| | anti-TNFR | | | VYYCARHGNFGNSYVSWFAYWGQGTLVTVS | LIGGTNKRAPGTPARF |
| | and | | | SASTKGPSVFPLAPCSRSTSESTAALGCLVKD | SGSLLGGKAALTLSGV |
| | anti-CD3 | | | YFPEPVTVSWNSGALTSGVHTFPAVLQSSGL | QPEDEAEYYCALWYSN |
| | antibody | | | YSLSSVVTVPSSSLGTKTYTCNVDHKPSNTKV | LWVFGGGTKLTVLGQP |
| | | | | DKRVESKYGPPCPPCPAPEAAGGPSVFLFPP | KAAPSVTLFPPSSEEL |
| | | | | KPKDTLMISRTPEVTCVVVDVSQEDPEVQFN | QANKATLVCLISDFYP |
| | | | | WYVDGVEVHNAKTKPREEQFNSTYRVVSVLT | GAVTVAWKADSSPVKA |
| | | | | VLHQDWLNGKEYKCKVSNKGLPSSIEKTISKA | GVETTTPSKQSNNKYA |
| | | | | KGQPREPQVYTLPPSQEEMTKNQVSLTCLVK | ASSYLSLTPEQWKSHR |
| | | | | GFYPSDIAVEWESNGQPENNYKTTPPVLDSD | SYSCQVTHEGSTVEKT |
| | | | | GSFLLYSKLTVDKSRWQEGNVFSCSVMHEAL | VAPTECS |
| | | | | HNHYTQKSLSLSLGK | |
| CD3 | teplizumab | 200 | 201 | QVQLVQSGGGVVQPGRSLRLSCKASGYTFTR | DIQMTQSPSSLSASVG |
| | | | | YTMHWVRQAPGKGLEWIGYINPSRGYTNYNQ | DRVTITCSASSSVSYM |
| | | | | KVKDRFTISRDNSKNTAFLQMDSLRPEDTGVY | NWYQQTPGKAPKRWIY |
| | | | | FCARYYDDHYCLDYWGQGTPVTVSSASTKGP | DTSKLASGVPSRFSGS |
| | | | | SVFPLAPSSKSTSGGTAALGCLVKDYFPEPVT | GSGTDYTFTISSLQPE |
| | | | | VSWNSGALTSGVHTFPAVLQSSGLYSLSSVVT | DIATYYCQQWSSNPFT |
| | | | | VPSSSLGTQTYICNVNHKPSNTKVDKKVEPKS | FGQGTKLQITRTVAAP |
| | | | | CDKTHTCPPCPAPEAAGGPSVFLFPPKPKDTL | SVFIFPPSDEQLKSGT |
| | | | | MISRTPEVTCVVVDVSHEDPEVKFNWYVDGV | ASVVCLLNNFYPREAK |
| | | | | EVHNAKTKPREEQYNSTYRVVSVLTVLHQDW | VQWKVDNALQSGNSQE |
| | | | | LNGKEYKCKVSNKALPAPIEKTISKAKGQPREP | SVTEQDSKDSTYSLSS |
| | | | | QVYTLPPSRDELTKNQVSLTCLVKGFYPSDIA | TLTLSKADYEKHKVYA |
| | | | | VEWESNGQPENNYKTTPPVLDSDGSFFLYSK | CEVTHQGLSSPVTKSF |
| | | | | LTVDKSRWQQGNVFSCSVMHEALHNHYTQK | NRGEC |
| | | | | SLSLSPGK | |
| CD3E | Tepodi- | 202 | | QVQLVQSGGGVVQPGRSLRLSCVASGFTFSS | |
| | tamab- | | | YGMHWVRQAPGKGLEWVAAIWYNARKQDYA | |
| | bispecfic | | | DSVKGRFTISRDNSKNTLYLQMNSLRAEDTAV | |
| | anti- | | | YYCTRGTGYNWFDPWGQGTLVTVSSASTKG | |
| | CLEC12A | | | PSVFPLAPSSKSTSGGTAALGCLVKDYFPEPV | |
| | and | | | TVSWNSGALTSGVHTFPAVLQSSGLYSLSSVV | |
| | anti-CD3E | | | TVPSSSLGTQTYICNVNHKPSNTKVDKRVEPK | |
| | | | | SCDKTHTCPPCPAPELGRGPSVFLFPPKPKDT | |
| | | | | LMISRTPEVTCVVVDVSHEDPEVKFNWYVDG | |
| | | | | VEVHNAKTKPREEQYNSTYRVVSVLTVLHQD | |
| | | | | WLNGKEYKCKVSNKALPAPIEKTISKAKGQPR | |
| | | | | EPQVYTKPPSREEMTKNQVSLKCLVKGFYPS | |
| | | | | DIAVEWESNGQPENNYKTTPPVLDSDGSFFLY | |
| | | | | SKLTVDKSRWQQGNVFSCSVMHEALHNHYT | |
| | | | | QKSLSLSPG | |
| CD3E | Tidutamab- | 203 | | EVQLVESGGGLVQPGGSLRLSCAASGFTFST | |
| | bi- | | | YAMNWVRQAPGKGLEWVGRIRSKYNNYATY | |
| | speicific | | | YADSVKGRFTISRDDSKNTLYLQMNSLRAEDT | |
| | anti-SSTR2 | | | AVYYCVRHGNFGDSYVSWFAYWGQGTLVTV | |
| | and anti- | | | SSGKPGSGKPGSGKPGSGKPGSQAVVTQEP | |
| | CD3E | | | SLTVSPGGTVTLTCGSSTGAVTTSNYANWVQ | |
| | | | | QKPGKSPRGLIGGTNKRAPGVPARFSGSLLG | |
| | | | | GKAALTISGAQPEDEADYYCALWYSNHWVF | |
| | | | | GGGTKLTVLEPKSSDKTHTCPPCPAPPVAGP | |
| | | | | SVFLFPPKPKDTLMISRTPEVTCVVVDVKHED | |
| | | | | PEVKFNWYVDGVEVHNAKTKPREEQYNSTYR | |
| | | | | VVSVLTVLHQDWLNGKEYKCKVSNKALPAPIE | |
| | | | | KTISKAKGQPREPQVYTLPPSREQMTKNQVKL | |
| | | | | TCLVKGFYPSDIAVEWESNGQPENNYKTTPPV | |
| | | | | LDSDGSFFLYSKLTVDKSRWQQGNVFSCSVM | |
| | | | | HEALHNHYTQKSLSLSPGK | |
| CD3 | TR66-CD3 | 204 | 205 | KLQQSGAELARPGASVKMSCKTSGYTFTRYT | IVLTQSPSIMSVSPGE |
| | binding | | | MHWVKQRPGQGLEWIGYINPSRGYTNYNQKF | KVTITCSASSSVSYMH |
| | fragment | | | KDKATLTTDKSSSTAYMQLSSLTSEDSAVYYC | WFQQKPGTSPKLSIYS |
| | | | | ARYYDDHYCLDYWGQGTTLTVSS | TSNLASGVPARFSGRG |
| | | | | | SGTSYSLTISRVAAED |
| | | | | | AATYYCQQRSNYPPWT |
| | | | | | FGGGTKLEIK |
| CD3 | Ubama- | 206 | | EVQLVESGGGLVQPGRSLRLSCAASGFTFDD | |
| | tamab- | | | YSMHWVRQAPGKGLEWVSGISWNSGSKGYA | |
| | bispecific | | | DSVKGRFTISRDNAKNSLYLQMNSLRAEDTAL | |
| | anti-MUC6 | | | YYCAKYGSGYGKFYHYGLDVWGQGTTVTVS | |
| | and | | | SASTKGPSVFPLAPCSRSTSESTAALGCLVKD | |
| | anti-CD3 | | | YFPEPVTVSWNSGALTSGVHTFPAVLQSSGL | |
| | | | | YSLSSVVTVPSSSLGTKTYTCNVDHKPSNTKV | |
| | | | | DKRVESKYGPPCPPCPAPPVAGPSVFLFPPK | |
| | | | | PKDTLMISRTPEVTCVVVDVSQEDPEVQFNW | |
| | | | | YVDGVEVHNAKTKPREEQFNSTYRVVSVLTVL | |
| | | | | HQDWLNGKEYKCKVSNKGLPSSIEKTISKAKG | |
| | | | | QPREPQVYTLPPSQEEMTKNQVSLTCLVKGF | |
| | | | | YPSDIAVEWESNGQPENNYKTTPPVLDSDGS | |
| | | | | FFLYSRLTVDKSRWQEGNVFSCSVMHEALHN | |
| | | | | RFTQKSLSLSPGK | |
| CD3 | UCHT1v9- | 207 | 208 | EVQLVESGGGLVQPGGSLRLSCAASGYSFTG | AIQMTQSPSSLSASVG |
| | CD3 | | | YTMNWVRQAPGKGLEWVALINPYKGVSTYNQ | DRVTITCRASQDIRNY |
| | binding | | | KFKDRFTISVDKSKNTAYLQMNSLRAEDTAVY | LNWYQQKPGKAPKLLI |
| | fragment | | | YCARSGYYGDSDWYFDVWGQGTLVTVSS | YYTSRLESGVPSRFSG |
| | | | | | SGSGTDYTLTISSLQP |
| | | | | | EDFATYYCQQGNTLPW |
| | | | | | TFGQGTKVEIK |
| CD3 | Vepsi- | 209 | | QVQLQQWGAGLLKPSETLSLTCAVYGGSFSG | |
| | tamab- | | | YYWSWIRQPPGKCLEWIGDIDASGSTKYNPSL | |
| | MUC17 | | | KSRVTISLDTSKNQFSLKLNSVTAADTAVYFCA | |
| | and | | | RKKYSTVWSYFDNWGQGTLVTVSSGGGGSG | |
| | CD3 BITE | | | GGGSGGGGSSYELTQPSSVSVPPGQTASITC | |
| | | | | SGDKLGDKYASWYQQKPGQSPVLVIYQDRKR | |
| | | | | PSGVPERFSGSNSGNTATLTISGTQAMDEAD | |
| | | | | YYCQAWGSSTAVFGCGTKLTVLSGGGGSEV | |
| | | | | QLVESGGGLVQPGGSLKLSCAASGFTFNKYA | |
| | | | | MNWVRQAPGKGLEWVARIRSKYNNYATYYA | |
| | | | | DSVKDRFTISRDDSKNTAYLQMNNLKTEDTAV | |
| | | | | YYCVRHGNFGNSYISYWAYWGQGTLVTVSSG | |
| | | | | GGGSGGGGSGGGGSQTVVTQEPSLTVSPGG | |
| | | | | TVTLTCGSSTGAVTSGNYPNWVQQKPGQAP | |
| | | | | RGLIGGTKFLAPGTPARFSGSLLGGKAALTL | |
| | | | | SGVQPEDEAEYYCVLWYSNRWVFGGGTKLT | |
| | | | | VLGGGGDKTHTCPPCPAPELLGGPSVFLFPP | |
| | | | | KPKDTLMISRTPEVTCVVVDVSHEDPEVKFNW | |
| | | | | YVDGVEVHNAKTKPCEEQYGSTYRCVSVLTV | |
| | | | | LHQDWLNGKEYKCKVSNKALPAPIEKTISKAK | |
| | | | | GQPREPQVYTLPPSREEMTKNQVSLTCLVKG | |
| | | | | FYPSDIAVEWESNGQPENNYKTTPPVLDSDG | |
| | | | | SFFLYSKLTVDKSRWQQGNVFSCSVMHEALH | |
| | | | | NHYTQKSLSLSPGKGGGGSGGGGSGGGGSG | |
| | | | | GGGSGGGGSGGGGSDKTHTCPPCPAPELLG | |
| | | | | GPSVFLFPPKPKDTLMISRTPEVTCVVVDVSH | |
| | | | | EDPEVKFNWYVDGVEVHNAKTKPCEEQYGST | |
| | | | | YRCVSVLTVLHQDWLNGKEYKCKVSNKALPA | |
| | | | | PIEKTISKAKGQPREPQVYTLPPSREEMTKNQ | |
| | | | | VSLTCLVKGFYPSDIAVEWESNGQPENNYKTT | |
| | | | | PPVLDSDGSFFLYSKLTVDKSRWQQGNVFSC | |
| | | | | SVMHEALHNHYTQKSLSLSPGK | |
| CD3E | Vibeco- | 210 | | EVQLVESGGGLVQPGGSLRLSCAASGFTFST | |
| | tamab- | | | YAMNWVRQAPGKGLEWVGRIRSKYNNYATY | |
| | bispecific | | | YADSVKGRFTISRDDSKNTLYLQMNSLRAEDT | |
| | anti-IL3R | | | AVYYCVRHGNFGDSYVSWFAYWGQGTLVTV | |
| | and | | | SSGKPGSGKPGSGKPGSGKPGSQAVVTQEP | |
| | anti-CD3E | | | SLTVSPGGTVTLTCGSSTGAVTTSNYANWVQ | |
| | antibody | | | QKPGKSPRGLIGGTNKRAPGVPARFSGSLLG | |
| | | | | GKAALTISGAQPEDEADYYCALWYSNHWVF | |
| | | | | GGGTKLTVLEPKSSDKTHTCPPCPAPPVAGP | |
| | | | | SVFLFPPKPKDTLMISRTPEVTCVVVDVKHED | |
| | | | | PEVKFNWYVDGVEVHNAKTKPREEQYNSTYR | |
| | | | | VVSVLTVLHQDWLNGKEYKCKVSNKALPAPIE | |
| | | | | KTISKAKGQPREPQVYTLPPSREQMTKNQVKL | |
| | | | | TCLVKGFYPSDIAVEWESNGQPENNYKTTPPV | |
| | | | | LDSDGSFFLYSKLTVDKSRWQQGNVFSCSVM | |
| | | | | HEALHNHYTQKSLSLSPGK | |
| CD3 | visili- | 211 | 212 | QVQLVQSGAEVKKPGASVKVSCKASGYTFISY | DIQMTQSPSSLSASVG |
| | zumab | | | TMHWVRQAPGQGLEWMGYINPRSGYTHYNQ | DRVTITCSASSSVSYM |
| | | | | KLKDKATLTADKSASTAYMELSSLRSEDTAVY | NWYQQKPGKAPKRLIY |
| | | | | YCARSAYYDYDGFAYWGQGTLVTVSSASTKG | DTSKLASGVPSRFSGS |
| | | | | PSVFPLAPCSRSTSESTAALGCLVKDYFPEPV | GSGTDFTLTISSLQPE |
| | | | | TVSWNSGALTSGVHTFPAVLQSSGLYSLSSVV | DFATYYCQQWSSNPPT |
| | | | | TVPSSNFGTQTYTCNVDHKPSNTKVDKTVER | FGGGTKVEIKRTVAAP |
| | | | | KCCVECPPCPAPPAAAPSVFLFPPKPKDTLMI | SVFIFPPSDEQLKSGT |
| | | | | SRTPEVTCVVVDVSHEDPEVQFNWYVDGVEV | ASVVCLLNNFYPREAK |
| | | | | HNAKTKPREEQFNSTFRVVSVLTVVHQDWLN | VQWKVDNALQSGNSQE |
| | | | | GKEYKCKVSNKGLPAPIEKTISKTKGQPREPQ | SVTEQDSKDSTYSLSS |
| | | | | VYTLPPSREEMTKNQVSLTCLVKGFYPSDIAV | TLTLSKADYEKHKVYA |
| | | | | EWESNGQPENNYKTTPPMLDSDGSFFLYSKL | CEVTHQGLSSPVTKSF |
| | | | | TVDKSRWQQGNVFSCSVMHEALHNHYTQKS | NRGEC |
| | | | | LSLSPSK | |
| CD3 | Vixtimo- | 213 | | DIQMTQSPSSLSASVGDRVTITCRSSTGAVTT | |
| | tamab- | | | SNYANWVQQKPGKAPKALIGGTNKRAPGVP | |
| | bispecific | | | SRFSGSLIGDKATLTISSLQPEDFATYYCALW | |
| | anti-CD3 | | | YSNLWVFGQGTKVEIKGGSGGSQVQLVQSG | |
| | anti-CD33 | | | AEVKKPGASVKVSCKASGYTFTSYDINWVRQ | |
| | antibody | | | APGQGLEWMGWMNPNSGNTGFAQKFQGRV | |
| | | | | TMTRDTSTSTVYMELSSLRSEDTAVYYCARD | |
| | | | | RANTDYSLGMDVWGQGTLVTVSSGGSGQSV | |
| | | | | LTQPPSASGTPGQRVTISCSGSRSNIGSNTVN | |
| | | | | WYQQLPGTAPKLLIYGNNQRPSGVPDRFSGS | |
| | | | | KSGTSASLAISGLQSEDEADYYCATWDDSLIG | |
| | | | | WVFGGGTKLTVLGGSGGSEVQLVESGGGLV | |
| | | | | QPGGSLRLSCAASGFTFSTYAMNWVRQAPG | |
| | | | | KGLEWVGRIRSKYNNYATYYADSVKDRFTISR | |
| | | | | DDSKNSLYLQMNSLKTEDTAVYYCARHGNFG | |
| | | | | NSYVSYFAYWGQGTLVTVSS | |
| CD3E | Voxala- | 214 | 215 | EVQLVESGGGLVQPGGSLRLSCAASGFTFNT | QTVVTQEPSLTVSPGG |
| | tamab- | | | YAMNWVRQAPGKGLEWVARIRSKYNNYATYY | TVTLTCRSSTGAVTTS |
| | bispecific | | | AASVKGRFTISRDDSKNSLYLQMNSLKTEDTA | NYANWVQQKPGQAPRG |
| | anti-PSMA | | | VYYCARHGNFGNSYVSWFAYWGQGTLVTVS | LIGGTNKRAPGTPARF |
| | and anti- | | | SASTKGPSVFPLAPCSRSTSESTAALGCLVKD | SGSLLGGKAALTLSGV |
| | CD3E | | | YFPEPVTVSWNSGALTSGVHTFPAVLQSSGL | QPEDEAEYYCALWYSN |
| | | | | YSLSSVVTVPSSSLGTKTYTCNVDHKPSNTKV | LWVFGGGTKLTVLGQP |
| | | | | DKRVESKYGPPCPPCPAPEAAGGPSVFLFPP | KAAPSVTLFPPSSEEL |
| | | | | KPKDTLMISRTPEVTCVVVDVSQEDPEVQFN | QANKATLVCLISDFYP |
| | | | | WYVDGVEVHNAKTKPREEQFNSTYRVVSVLT | GAVTVAWKADSSPVKA |
| | | | | VLHQDWLNGKEYKCKVSNKGLPSSIEKTISKA | GVETTTPSKQSNNKYA |
| | | | | KGQPREPQVYTLPPSQEEMTKNQVSLTCLVK | ASSYLSLTPEQWKSHR |
| | | | | GFYPSDIAVEWESNGQPENNYKTTPPVLDSD | SYSCQVTHEGSTVEKT |
| | | | | GSFLLYSKLTVDKSRWQEGNVFSCSVMHEAL | VAPTECS |
| | | | | HNHYTQKSLSLSLGK | |
| CD3 | XR32 | 216 | 217 | EVQLVESGGGLVQPGGSLRLSCAASGFTFNT | QAVVTQEPSLTVSPGG |
| | | | | YAMNWVRQAPGKGLEWVARIRSKYNNYATYY | TVTLTCRSSTGAVTTS |
| | | | | ADSVKDRFTISRDDSKNSLYLQMNSLKTEDTA | NYANWVQQKPGQAPRG |
| | | | | VYYCVRHGNFGNSYVSWFAYWGQGTLVTVS | LIGGTNKRAPWTPARF |
| | | | | S | SGSLLGGKAALTITGA |
| | | | | | QAEDEADYYCALWYSN |
| | | | | | LWVFGGGTKLTVL |
| CD3 | OKT3 | 218 | 219 | QVQLQQSGAELARPGASVKMSCKASGYTFTR | QIVLTQSPAIMSASPG |
| | | | | YTMHWVKQRPGQGLEWIGYINPSRGYTNYNQ | EKVTMTCSASSSVSYM |
| | | | | KFKDKATLTTDKSSSTAYMQLSSLTSEDSAVY | NWYQQKSGTSPKRWIY |
| | | | | YCARYYDDHYCLDYWGQGTTLTVSSAKTTAP | DTSKLASGVPAHFRGS |
| | | | | SVYPLAPVCGGTTGSSVTLGCLVKGYFPEPVT | GSGTSYSLTISGMEAE |
| | | | | LTWNSGSLSSGVHTFPAVLQSDLYTLSSSVTV | DAATYYCQQWSSNPFT |
| | | | | TSSTWPSQSITCNVAHPASSTKVDKKIEPRPK | FGSGTKLEINRADTAP |
| | | | | SCDKTHTCPPCPAPELLGGPSVFLFPPKPKDT | TVSIFPPSSEQLTSGG |
| | | | | LMISRTPEVTCVVVDVSHEDPEVKFNWYVDG | ASVVCFLNNFYPKDIN |
| | | | | VEVHNAKTKPREEQYNSTYRVVSVLTVLHQD | VKWKIDGSERQNGVLN |
| | | | | WLNGKEYKCKVSNKALPAPIEKTISKAKGQPR | SWTDQDSKDSTYSMSS |
| | | | | EPQVYTLPPSRDELTKNQVSLTCLVKGFYPSD | TLTLTKDEYERHNSYT |
| | | | | IAVEWESNGQPENNYKTTPPVLDSDGSFFLYS | CEATHKTSTSPIVKSF |
| | | | | KLTVDKSRWQQGNVFSCSVMHEALHNHYTQ | NRNEC |
| | | | | KSLSLSPGK | |
| CD4 | ibalizumab | 220 | 221 | QVQLQQSGPEVVKPGASVKMSCKASGYTFTS | DIVMTQSPDSLAVSLG |
| | | | | YVIHWVRQKPGQGLDWIGYINPYNDGTDYDE | ERVTMNCKSSQSLLYS |
| | | | | KFKGKATLTSDTSTSTAYMELSSLRSEDTAVY | TNQKNYLAWYQQKPGQ |
| | | | | YCAREKDNYATGAWFAYWGQGTLVTVSSAST | SPKLLIYWASTRESGV |
| | | | | KGPSVFPLAPCSRSTSESTAALGCLVKDYFPE | PDRFSGSGSGTDFTLT |
| | | | | PVTVSWNSGALTSGVHTFPAVLQSSGLYSLSS | ISSVQAEDVAVYYCQQ |
| | | | | VVTVPSSSLGTKTYTCNVDHKPSNTKVDKRVE | YYSYRTFGGGTKLEIK |
| | | | | SKYGPPCPSCPAPEFLGGPSVFLFPPKPKDTL | RTVAAPSVFIFPPSDE |
| | | | | MISRTPEVTCVVVDVSQEDPEVQFNWYVDGV | QLKSGTASVVCLLNNF |
| | | | | EVHNAKTKPREEQFNSTYRVVSVLTVLHQDW | YPREAKVQWKVDNALQ |
| | | | | LNGKEYKCKVSNKGLPSSIEKTISKAKGQPRE | SGNSQESVTEQDSKDS |
| | | | | PQVYTLPPSQEEMTKNQVSLTCLVKGFYPSDI | TYSLSSTLTLSKADYE |
| | | | | AVEWESNGQPENNYKTTPPVLDSDGSFFLYS | KHKVYACEVTHQGLSS |
| | | | | RLTVDKSRWQEGNVFSCSVMHEALHNHYTQ | PVTKSFNRGEC |
| | | | | KSLSLSLGK | |
| CD4 | inezetamab | 222 | 223 | EVQLLESGGGLVQPGGSLRLSCAASGFTFSR | QSALTQPASVSGSPGQ |
| | | | | NAMSWVRQAPGKGLEWVSATGGSGISTYYA | SITISCTGTSSDVGNY |
| | | | | DSVKGRFTISRDNSKNTLYLQMNSLRAEDTAV | NLVSWYQQHPGKAPKL |
| | | | | YYCARGYSNSWWYFDYWGQGTLVTVSSAST | MIFEVNQRPSGVSNRF |
| | | | | KGPSVFPLAPSSKSTSGGTAALGCLVKDYFPE | SGSKSGTTASLTISGL |
| | | | | PVTVSWNSGALTSGVHTFPAVLQSSGLYSLSS | QAADEADYFCSSYTTS |
| | | | | VVTVPSSSLGTQTYICNVNHKPSNTKVDKKVE | STYVIFGGGTKLTVLG |
| | | | | PKSCDKTHTCPPCPAPELLGGPSVFLFPPKPK | QPKAAPSVTLFPPSSE |
| | | | | DTLMISRTPEVTCVVVDVSHEDPEVKFNWYVD | ELQANKATLVCLISDF |
| | | | | GVEVHNAKTKPCEEQYGSTYRCVSVLTVLHQ | YPGAVTVAWKADSSPV |
| | | | | DWLNGKEYKCKVSNKALPAPIEKTISKAKGQP | KAGVETTTPSKQSNNK |
| | | | | REPQVYTLPPSREEMTKNQVSLTCLVKGFYPS | YAASSYLSLTPEQWKS |
| | | | | DIAVEWESNGQPENNYKTTPPVLDSDGSFFLY | HRSYSCQVTHEGSTVE |
| | | | | SKLTVDKSRWQQGNVFSCSVMHEALHNHYT | KTVAPTECS |
| | | | | QKSLSLSPGGGGSGGGGSQVQLVESGGGLV | |
| | | | | KPGGSLRLSCAASGFTFSDYYMSWIRQAPGK | |
| | | | | CLEWISYISSSESIIYYVDAVKGRFTISRDNAK | |
| | | | | NSLYLQMNSLRAEDTAVYYCARDVGSHFDYWG | |
| | | | | QGTLVTVSSGGGGSGGGGSGGGGSDIQMTQ | |
| | | | | SPSSVSASVGDRVTITCRASQDISRWLAWYQ | |
| | | | | QKPGKAPKLLISAASRLQSGVPSRFSGSGSGT | |
| | | | | DFTLTISSLQPEDFAIYYCQQAKSFPRTFGCGT | |
| | | | | KVEIKR | |
| CD4 | semzuvo- | 224 | 225 | QVQLVQSGPELKKPGASVKVSCKASGYTFTD | DIVLTQSPASLAVSLG |
| | limab | | | YVIHWVKQATGQGLEWIGEIYPGSGSAYSNAK | QRATITCKAGQSVDYD |
| | | | | FKDRVTMTADKSSNTAYMELSSLTSDDTAVYF | GDSYMNWYQQKPGQPP |
| | | | | CARRGNGTGFAYWGQGTLVTVSSASTKGPS | KLLIYVASNLESGIPA |
| | | | | VFPLAPSSKSTSGGTAALGCLVKDYFPEPVTV | RFSGSGSGTDFTLNIH |
| | | | | SWNSGALTSGVHTFPAVLQSSGLYSLSSVVTV | PVEENDAATYYCQQSY |
| | | | | PSSSLGTQTYICNVNHKPSNTKVDKKVEPKSC | KDPLTFGQGTKLEIKR |
| | | | | DKTHTCPPCPAPELLGGPSVFLFPPKPKDTLM | TVAAPSVFIFPPSDEQ |
| | | | | ISRTPEVTCVVVDVSHEDPEVKFNWYVDGVE | LKSGTASVVCLLNNFY |
| | | | | VHNAKTKPREEQYHSTYRVVSVLTVLHQDWL | PREAKVQWKVDNALQS |
| | | | | NGKEYKCKVSNKALPAPIEKTISKAKGQPREP | GNSQESVTEQDSKDST |
| | | | | QVYTLPPSRDELTKNQVSLTCLVKGFYPSDIA | YSLSSTLTLSKADYEK |
| | | | | VEWESNGQPENNYKTTPPVLDSDGSFFLYSK | HKLYACEVTHQGLSSP |
| | | | | LTVDKSRWQQGNVFSCSVMHEALHNHYTQK | VTKSFNRGEC |
| | | | | SLSLSPGK | |
| CD4 | tregali- | 226 | 227 | EEQLVESGGGLVKPGGSLRLSCAASGFSFSD | DIVMTQSPDSLAVSLG |
| | zumab | | | CRMYWLRQAPGKGLEWIGVISVKSENYGANY | ERATINCRASKSVSTS |
| | | | | AESVRGRFTISRDDSKNTVYLQMNSLKTEDTA | GYSYIYWYQQKPGQPP |
| | | | | VYYCSASYYRYDVGAWFAYWGQGTLVTVSS | KLLIYLASILESGVPD |
| | | | | ASTKGPSVFPLAPSSKSTSGGTAALGCLVKDY | RFSGSGSGTDFTLTIS |
| | | | | FPEPVTVSWNSGALTSGVHTFPAVLQSSGLY | SLQAEDVAVYYCQHSR |
| | | | | SLSSVVTVPSSSLGTQTYICNVNHKPSNTKVD | ELPWTFGQGTKVEIKR |
| | | | | KKVEPKSCDKTHTCPPCPAPELLGGPSVFLFP | TVAAPSVFIFPPSDEQ |
| | | | | PKPKDTLMISRTPEVTCVVVDVSHEDPEVKFN | LKSGTASVVCLLNNFY |
| | | | | WYVDGVEVHNAKTKPREEQYNSTYRVVSVLT | PREAKVQWKVDNALQS |
| | | | | VLHQDWLNGKEYKCKVSNKALPAPIEKTISKA | GNSQESVTEQDSKDST |
| | | | | KGQPREPQVYTLPPSRDELTKNQVSLTCLVK | YSLSSTLTLSKADYEK |
| | | | | GFYPSDIAVEWESNGQPENNYKTTPPVLDSD | HKVYACEVTHQGLSSP |
| | | | | GSFFLYSKLTVDKSRWQQGNVFSCSVMHEAL | VTKSFNRGEC |
| | | | | HNHYTQKSLSLSPGK | |
| CD4 | Zanoli- | 506 | 507 | QVQLQQWGAGLLKPSETLSLTCAVYGGSFSG | DIQMTQSPSSVSASVG |
| | mumab- | | | YYWSWIRQPPGKGLEWIGEINHSGSTNYNPS | DRVTITCRASQDISSW |
| | CD4 | | | LKSRVTISVDTSKNQFSLKLSSVTAADTAVYYC | LAWYQHKPGKAPKLLI |
| | binding | | | ARVINWFDPWGQGTLVT | YAASSLQSGVPSRFSG |
| | fragment | | | | SGSGTDFTLTISSLQP |
| | | | | | EDFATYYCQQANSFPY |
| | | | | | TFGQGTKLEIK |
| CD40 | bleselumab | 228 | 229 | QLQLQESGPGLLKPSETLSLTCTVSGGSISSP | AIQLTQSPSSLSASVG |
| | | | | GYYGGWIRQPPGKGLEWIGSIYKSGSTYHNP | DRVTITCRASQGISSA |
| | | | | SLKSRVTISVDTSKNQFSLKLSSVTAADTAVYY | LAWYQQKPGKAPKLLI |
| | | | | CTRPVVRYFGWFDPWGQGTLVTVSSASTKG | YDASNLESGVPSRFSG |
| | | | | PSVFPLAPCSRSTSESTAALGCLVKDYFPEPV | SGSGTDFTLTISSLQP |
| | | | | TVSWNSGALTSGVHTFPAVLQSSGLYSLSSVV | EDFATYYCQQFNSYPT |
| | | | | TVPSSSLGTKTYTCNVDHKPSNTKVDKRVESK | FGQGTKVEIKRTVAAP |
| | | | | YGPPCPPCPAPEFEGGPSVFLFPPKPKDTLMI | SVFIFPPSDEQLKSGT |
| | | | | SRTPEVTCVVVDVSQEDPEVQFNWYVDGVEV | ASVVCLLNNFYPREAK |
| | | | | HNAKTKPREEQFNSTYRVVSVLTVLHQDWLN | VQWKVDNALQSGNSQE |
| | | | | GKEYKCKVSNKGLPSSIEKTISKAKGQPREPQ | SVTEQDSKDSTYSLSS |
| | | | | VYTLPPSQEEMTKNQVSLTCLVKGFYPSDIAV | TLTLSKADYEKHKVYA |
| | | | | EWESNGQPENNYKTTPPVLDSDGSFFLYSRL | CEVTHQGLSSPVTKSF |
| | | | | TVDKSRWQEGNVFSCSVMHEALHNHYTQKSL | NRGEC |
| | | | | SLSLGK | |
| CD40 | cifurti- | 230 | 231 | EVQLVESGGGLVQPGGSLRLSCAASGYSFTG | DIQMTQSPSSLSASVG |
| | limab | | | YYIHWVRQAPGKGLEWVARVIPNAGGTSYNQ | DRVTITCRSSQSLVHS |
| | | | | KFKGRFTLSVDNSKNTAYLQMNSLRAEDTAVY | NGNTFLHWYQQKPGKA |
| | | | | YCAREGIYWWGQGTLVTVSSASTKGPSVFPL | PKLLIYTVSNRFSGVP |
| | | | | APSSKSTSGGTAALGCLVKDYFPEPVTVSWN | SRFSGSGSGTDFTLTI |
| | | | | SGALTSGVHTFPAVLQSSGLYSLSSVVTVPSS | SSLQPEDFATYFCSQT |
| | | | | SLGTQTYICNVNHKPSNTKVDKKVEPKSCDKT | THVPWTFGQGTKVEIK |
| | | | | HTCPPCPAPELLGGPSVFLFPPKPKDTLMISR | RTVAAPSVFIFPPSDE |
| | | | | TPEVTCVVVDVSHEDPEVKFNWYVDGVEVHN | QLKSGTASVVCLLNNF |
| | | | | AKTKPREEQYNSTYRVVSVLTVLHQDWLNGK | YPREAKVQWKVDNALQ |
| | | | | EYKCKVSNKALPAPIEKTISKAKGQPREPQVYT | SGNSQESVTEQDSKDS |
| | | | | LPPSREEMTKNQVSLTCLVKGFYPSDIAVEWE | TYSLSSTLTLSKADYE |
| | | | | SNGQPENNYKTTPPVLDSDGSFFLYSKLTVDK | KHKVYACEVTHQGLSS |
| | | | | SRWQQGNVFSCSVMHEALHNHYTQKSLSLSP | PVTKSFNRGEC |
| | | | | GK | |
| CD40 | dacetu- | 232 | 233 | EVQLVESGGGLVQPGGSLRLSCAASGYSFTG | DIQMTQSPSSLSASVG |
| | zumab | | | YYIHWVRQAPGKGLEWVARVIPNAGGTSYNQ | DRVTITCRSSQSLVHS |
| | | | | KFKGRFTLSVDNSKNTAYLQMNSLRAEDTAVY | NGNTFLHWYQQKPGKA |
| | | | | YCAREGIYWWGQGTLVTVSSASTKGPSVFPL | PKLLIYTVSNRFSGVP |
| | | | | APSSKSTSGGTAALGCLVKDYFPEPVTVSWN | SRFSGSGSGTDFTLTI |
| | | | | SGALTSGVHTFPAVLQSSGLYSLSSVVTVPSS | SSLQPEDFATYFCSQT |
| | | | | SLGTQTYICNVNHKPSNTKVDKKVEPKSCDKT | THVPWTFGQGTKVEIK |
| | | | | HTCPPCPAPELLGGPSVFLFPPKPKDTLMISR | RTVAAPSVFIFPPSDE |
| | | | | TPEVTCVVVDVSHEDPEVKFNWYVDGVEVHN | QLKSGTASVVCLLNNF |
| | | | | AKTKPREEQYNSTYRVVSVLTVLHQDWLNGK | YPREAKVQWKVDNALQ |
| | | | | EYKCKVSNKALPAPIEKTISKAKGQPREPQVYT | SGNSQESVTEQDSKDS |
| | | | | LPPSREEMTKNQVSLTCLVKGFYPSDIAVEWE | TYSLSSTLTLSKADYE |
| | | | | SNGQPENNYKTTPPVLDSDGSFFLYSKLTVDK | KHKVYACEVTHQGLSS |
| | | | | SRWQQGNVFSCSVMHEALHNHYTQKSLSLSP | PVTKSFNRGEC |
| | | | | GK | |
| CD40 | gilora- | 234 | 235 | EVQLQESGPGLVKPSETLSLTCTVSGYSITSN | DIVMTQTPLSLSVTPG |
| | limab | | | YYWNWIRQPPGKGLEWMGYIRYDGSNNYNP | QPASISCRSSQSLENT |
| | | | | SLKNRVTISRDTSKNQFSLKLSSVTAADTAVYY | NGNTFLNWYLQKPGQS |
| | | | | CARLDYWGQGTTVTVSSASTKGPSVFPLAPS | PQLLIYRVSNRFSGVP |
| | | | | SKSTSGGTAALGCLVKDYFPEPVTVSWNSGA | DRFSGSGSGTDFTLKI |
| | | | | LTSGVHTFPAVLQSSGLYSLSSVVTVPSSSLG | SRVEAEDVGVYYCLQV |
| | | | | TQTYICNVNHKPSNTKVDKKVEPKSCDKTHTC | THVPFTFGQGTKLEIK |
| | | | | PPCPAPELLGGPSVFLFPPKPKDTLMISRTPEV | RTVAAPSVFIFPPSDE |
| | | | | TCVVVDVSHEDPEEKFNWYVDGVEVHNAKTK | QLKSGTASVVCLLNNF |
| | | | | PREEQYNSTYRVVSVLTVLHQDWLNGKEYKC | YPREAKVQWKVDNALQ |
| | | | | KVSNKALPAPIEKTISKAKGQPREPQVYTLPPS | SGNSQESVTEQDSKDS |
| | | | | REEMTKNQVSLTCLVKGFYPSDIAVEWESNG | TYSLSSTLTLSKADYE |
| | | | | QPENNYKTTPPVLDSDGSFFLYSKLTVDKSR | KHKVYACEVTHQGLSS |
| | | | | WQQGNVFSCSVMHEALHNHYTQKSLSLSPG | PVTKSFNRGEC |
| | | | | K | |
| CD40 | iscalimab | 236 | 237 | QVQLVESGGGVVQPGRSLRLSCAASGFTFSS | DIVMTQSPLSLTVTPG |
| | | | | YGMHWVRQAPGKGLEWVAVISYEESNRYHA | EPASISCRSSQSLLYS |
| | | | | DSVKGRFTISRDNSKITLYLQMNSLRTEDTAVY | NGYNYLDWYLQKPGQS |
| | | | | YCARDGGIAAPGPDYWGQGTLVTVSSASTKG | PQVLISLGSNRASGVP |
| | | | | PSVFPLAPSSKSTSGGTAALGCLVKDYFPEPV | DRFSGSGSGTDFTLKI |
| | | | | TVSWNSGALTSGVHTFPAVLQSSGLYSLSSVV | SRVEAEDVGVYYCMQA |
| | | | | TVPSSSLGTQTYICNVNHKPSNTKVDKRVEPK | RQTPFTFGPGTKVDIR |
| | | | | SCDKTHTCPPCPAPELLGGPSVFLFPPKPKDT | RTVAAPSVFIFPPSDE |
| | | | | LMISRTPEVTCVVVDVSHEDPEVKFNWYVDG | QLKSGTASVVCLLNNF |
| | | | | VEVHNAKTKPREEQYASTYRVVSVLTVLHQD | YPREAKVQWKVDNALQ |
| | | | | WLNGKEYKCKVSNKALPAPIEKTISKAKGQPR | SGNSQESVTEQDSKDS |
| | | | | EPQVYTLPPSREEMTKNQVSLTCLVKGFYPSD | TYSLSSTLTLSKADYE |
| | | | | IAVEWESNGQPENNYKTTPPVLDSDGSFFLYS | KHKVYACEVTHQGLSS |
| | | | | KLTVDKSRWQQGNVFSCSVMHEALHNHYTQ | PVTKSFNRGEC |
| | | | | KSLSLSPGK | |
| CD40 | lucatu- | 238 | 239 | QVQLVESGGGVVQPGRSLRLSCAASGFTFSS | DIVMTQSPLSLTVTPG |
| | mumab | | | YGMHWVRQAPGKGLEWVAVISYEESNRYHA | EPASISCRSSQSLLYS |
| | | | | DSVKGRFTISRDNSKITLYLQMNSLRTEDTAVY | NGYNYLDWYLQKPGQS |
| | | | | YCARDGGIAAPGPDYWGQGTLVTVSSASTKG | PQVLISLGSNRASGVP |
| | | | | PSVFPLAPASKSTSGGTAALGCLVKDYFPEPV | DRFSGSGSGTDFTLKI |
| | | | | TVSWNSGALTSGVHTFPAVLQSSGLYSLSSVV | SRVEAEDVGVYYCMQA |
| | | | | TVPSSSLGTQTYICNVNHKPSNTKVDKRVEPK | RQTPFTFGPGTKVDIR |
| | | | | SCDKTHTCPPCPAPELLGGPSVFLFPPKPKDT | RTVAAPSVFIFPPSDE |
| | | | | LMISRTPEVTCVVVDVSHEDPEVKFNWYVDG | QLKSGTASVVCLLNNF |
| | | | | VEVHNAKTKPREEQYNSTYRVVSVLTVLHQD | YPREAKVQWKVDNALQ |
| | | | | WLNGKEYKCKVSNKALPAPIEKTISKAKGQPR | SGNSQESVTEQDSKDS |
| | | | | EPQVYTLPPSREEMTKNQVSLTCLVKGFYPSD | TYSLSSTLTLSKADYE |
| | | | | IAVEWESNGQPENNYKTTPPVLDSDGSFFLYS | KHKVYACEVTHQGLSS |
| | | | | KLTVDKSRWQQGNVFSCSVMHEALHNHYTQ | PVTKSFNRGEC |
| | | | | KSLSLSPGK | |
| CD40 | mitaza- | 240 | 241 | EVQLLESGGGLVQPGGSLRLSCAASGFTFST | QSVLTQPPSASGTPGQ |
| | limab | | | YGMHWVRQAPGKGLEWLSYISGGSSYIFYAD | RVTISCTGSSSNIGAG |
| | | | | SVRGRFTISRDNSENALYLQMNSLRAEDTAVY | YNVYWYQQLPGTAPKL |
| | | | | YCARILRGGSGMDLWGQGTLVTVSSASTKGP | LIYGNINRPSGVPDRF |
| | | | | SVFPLAPSSKSTSGGTAALGCLVKDYFPEPVT | SGSKSGTSASLAISGL |
| | | | | VSWNSGALTSGVHTFPAVLQSSGLYSLSSVVT | RSEDEADYYCAAWDKS |
| | | | | VPSSSLGTQTYICNVNHKPSNTKVDKKVEPKS | ISGLVFGGGTKLTVLG |
| | | | | CDKTHTCPPCPAPELLGGPSVFLFPPKPKDTL | QPKAAPSVTLFPPSSE |
| | | | | MISRTPEVTCVVVDVSHEDPEVKFNWYVDGV | ELQANKATLVCLISDF |
| | | | | EVHNAKTKPREEQYNSTYRVVSVLTVLHQDW | YPGAVTVAWKADSSPV |
| | | | | LNGKEYKCKVSNKALPAPIEKTISKAKGQPREP | KAGVETTTPSKQSNNK |
| | | | | QVYTLPPSRDELTKNQVSLTCLVKGFYPSDIA | YAASSYLSLTPEQWKS |
| | | | | VEWESNGQPENNYKTTPPVLDSDGSFFLYSK | HRSYSCQVTHEGSTVE |
| | | | | LTVDKSRWQQGNVFSCSVMHEALHNHYTQK | KTVAPTECS |
| | | | | SLSLSPGK | |
| CD40 | ravaga- | 242 | 243 | EVQLVESGGGLVKPGGSLRLSCAASGFTFSD | DIVMTQSPDSLAVSLG |
| | limab | | | YGMNWVRQAPGKGLEWIAYISSGRGNIYYAD | ERATINCKSSQSLLNR |
| | | | | TVKGRFTISRDNAKNSLYLQMNSLRAEDTAVY | GNQKNYLTWFQQKPGQ |
| | | | | YCARSWGYFDVWGQGTTVTVSSASTKGPSV | PPKLLIYWASTRESGV |
| | | | | FPLAPSSKSTSGGTAALGCLVKDYFPEPVTVS | PDRFSGSGSGTDFTLT |
| | | | | WNSGALTSGVHTFPAVLQSSGLYSLSSVVTVP | ISSLQAEDVAVYYCQN |
| | | | | SSSLGTQTYICNVNHKPSNTKVDKKVEPKSCD | DYTYPLTFGQGTKLEI |
| | | | | KTHTCPPCPAPEAAGGPSVFLFPPKPKDQLMI | KRTVAAPSVFIFPPSD |
| | | | | SRTPEVTCVVVDVSHEDPEVKFNWYVDGVEV | EQLKSGTASVVCLLNN |
| | | | | HNAKTKPREEQYNSTYRVVSVLTVLHQDWLN | FYPREAKVQWKVDNAL |
| | | | | GKEYKCKVSNKALPAPIEKTISKAKGQPREPQ | QSGNSQESVTEQDSKD |
| | | | | VYTLPPSREEMTKNQVSLTCLVKGFYPSDIAV | STYSLSSTLTLSKADY |
| | | | | EWESNGQPENNYKTTPPVLDSDGSFFLYSKL | EKHKVYACEVTHQGLS |
| | | | | TVDKSRWQQGNVFSCSVLHEALHNHYTQKSL | SPVTKSFNRGEC |
| | | | | SLSPGK | |
| CD40 | selicre- | 244 | 245 | QVQLVQSGAEVKKPGASVKVSCKASGYTFTG | DIQMTQSPSSVSASVG |
| | lumab | | | YYMHWVRQAPGQGLEWMGWINPDSGGTNY | DRVTITCRASQGIYSW |
| | | | | AQKFQGRVTMTRDTSISTAYMELNRLRSDDTA | LAWYQQKPGKAPNLLI |
| | | | | VYYCARDQPLGYCTNGVCSYFDYWGQGTLV | YTASTLQSGVPSRFSG |
| | | | | TVSSASTKGPSVFPLAPCSRSTSESTAALGCL | SGSGTDFTLTISSLQP |
| | | | | VKDYFPEPVTVSWNSGALTSGVHTFPAVLQS | EDFATYYCQQANIFPL |
| | | | | SGLYSLSSVVTVPSSNFGTQTYTCNVDHKPSN | TFGGGTKVEIKRTVAA |
| | | | | TKVDKTVERKCCVECPPCPAPPVAGPSVFLFP | PSVFIFPPSDEQLKSG |
| | | | | PKPKDTLMISRTPEVTCVVVDVSHEDPEVQFN | TASVVCLLNNFYPREA |
| | | | | WYVDGVEVHNAKTKPREEQFNSTFRVVSVLT | KVQWKVDNALQSGNSQ |
| | | | | VVHQDWLNGKEYKCKVSNKGLPAPIEKTISKT | ESVTEQDSKDSTYSLS |
| | | | | KGQPREPQVYTLPPSREEMTKNQVSLTCLVK | STLTLSKADYEKHKVY |
| | | | | GFYPSDIAVEWESNGQPENNYKTTPPMLDSD | ACEVTHQGLSSPVTKS |
| | | | | GSFFLYSKLTVDKSRWQQGNVFSCSVMHEAL | FNRGEC |
| | | | | HNHYTQKSLSLSPGK | |
| CD40 | sotiga- | 246 | 247 | QVQLVESGGGVVQPGRSLRLSCAASGFSFSS | DIQMTQSPSSLSASVG |
| | limab | | | TYVCWVRQAPGKGLEWIACIYTGDGTNYSAS | DRVTIKCQASQSISSR |
| | | | | WAKGRFTISKDSSKNTVYLQMNSLRAEDTAVY | LAWYQQKPGKPPKLLI |
| | | | | FCARPDITYGFAINFWGPGTLVTVSSASTKGP | YRASTLASGVPSRFSG |
| | | | | SVFPLAPSSKSTSGGTAALGCLVKDYFPEPVT | SGSGTDFTLTISSLQP |
| | | | | VSWNSGALTSGVHTFPAVLQSSGLYSLSSVVT | EDVATYYCQCTGYGIS |
| | | | | VPSSSLGTQTYICNVNHKPSNTKVDKKVEPKS | WPIGGGTKVEIKRTVA |
| | | | | CDKTHTCPPCPAPELLGGPSVFLFPPKPKDTL | APSVFIFPPSDEQLKS |
| | | | | MISRTPEVTCVVVDVEHEDPEVKFNWYVDGV | GTASVVCLLNNFYPRE |
| | | | | EVHNAKTKPREEQYNSTYRVVSVLTVLHQDW | AKVQWKVDNALQSGNS |
| | | | | LNGKEYKCKVSNKALPAPIEKTISKAKGQPREP | QESVTEQDSKDSTYSL |
| | | | | QVYTLPPSRDELTKNQVSLTCLVKGFYPSDIA | SSTLTLSKADYEKHKV |
| | | | | VEWESNGQPENNYKTTPPVLDSDGSFFLYSK | YACEVTHQGLSSPVTK |
| | | | | LTVDKSRWQQGNVFSCSVMHEALHNHYTQK | SFNRGEC |
| | | | | SLSLSPGK | |
| CD40 | tecagin- | 248 | 249 | EVQLVQSGAEVKKPGASVKVSCKTSGYTFTE | DIQMTQSPSSLSASVG |
| | limab | | | YIMHWVRQAPGQGLEWMGGIIPNNGGTSYNQ | DRVTITCSASQGINNY |
| | | | | KFQGRVTMTVDKSTSTGYMELSSLRSEDTAV | LNWYQQKPGKAVKLLI |
| | | | | YYCTRREVYGRNYYALDYWGQGTLVTVSSAS | YYTSSLHSGVPSRFSG |
| | | | | TKGPSVFPLAPSSKSTSGGTAALGCLVKDYFP | SGSGTDYTFTISSLQP |
| | | | | EPVTVSWNSGALTSGVHTFPAVLQSSGLYSLS | EDIATYYCQQYSNLPY |
| | | | | SVVTVPSSSLGTQTYICNVNHKPSNTKVDKRV | TFGGGTKVEIKRTVAA |
| | | | | EPKSCDKTHTCPPCPAPEFEGGPSVFLFPPKP | PSVFIFPPSDEQLKSG |
| | | | | KDTLMISRTPEVTCVVVAVSHEDPEVKFNWYV | TASVVCLLNNFYPREA |
| | | | | DGVEVHNAKTKPREEQYNSTYRVVSVLTVLH | KVQWKVDNALQSGNSQ |
| | | | | QDWLNGKEYKCKVSNKALPAPIEKTISKAKGQ | ESVTEQDSKDSTYSLS |
| | | | | PREPQVYTLPPSREEMTKNQVSLTCLVKGFYP | STLTLSKADYEKHKVY |
| | | | | SDIAVEWESNGQPENNYKTTPPVLDSDGSFLL | ACEVTHQGLSSPVTKS |
| | | | | YSKLTVDKSRWQQGNVFSCSVMHEALHNHYT | FNRGEC |
| | | | | QKSLSLSP | |
| CD40 | tenelix- | 250 | 251 | QIQLVQSGPELKKPGETVRISCKASGYAFTTT | DIVLTQSPATLSVTPG |
| | imab | | | GMQWVQEMPGKGLKWIGWINTHSGVPKYVE | DRVSLSCRASQSISDY |
| | | | | DFKGRFAFSLETSANTAYLQISNLKNEDTATYF | LHWYQQKSHESPRLLI |
| | | | | CVRSGNGNYDLAYFAYWGQGTLVTVSAASTK | KYASHSISGIPSRFSG |
| | | | | GPSVFPLAPSSKSTSGGTAALGCLVKDYFPEP | SGSGSDFTLSINSVEP |
| | | | | VTVSWNSGALTSGVHTFPAVLQSSGLYSLSSV | EDVGIYYCQHGHSFPW |
| | | | | VTVPSSSLGTQTYICNVNHKPSNTKVDKKVEP | TFGGGTKLEIKRTVAA |
| | | | | KSCDKTHTCPPCPAPELLGGPSVFLFPPKPKD | PSVFIFPPSDEQLKSG |
| | | | | TLMISRTPEVTCVVVDVSHEDPEVKFNWYVD | TASVVCLLNNFYPREA |
| | | | | GVEVHNAKTKPREEQYNSTYRVVSVLTVLHQ | KVQWKVDNALQSGNSQ |
| | | | | DWLNGKEYKCKVSNKALPAPIEKTISKAKGQP | ESVTEQDSKDSTYSLS |
| | | | | REPQVYTLPPSRDELTKNQVSLTCLVKGFYPS | STLTLSKADYEKHKVY |
| | | | | DIAVEWESNGQPENNYKTTPPVLDSDGSFFLY | ACEVTHQGLSSPVTKS |
| | | | | SKLTVDKSRWQQGNVFSCSVMHEALHNHYT | FNRGEC |
| | | | | QKSLSLSPGK | |
| CD40L | dapirol- | 252 | 253 | EVQLVESGGGLVQPGGSLRLSCAVSGFSSTN | DIQMTQSPSSLSASVG |
| | izumab | | | YHVHWVRQAPGKGLEWMGVIWGDGDTSYNS | DRVTITCRASEDLYYN |
| | | | | VLKSRFTISRDTSKNTVYLQMNSLRAEDTAVY | LAWYQRKPGKAPKLLI |
| | | | | YCARQLTHYYVLAAWGQGTLVTVSSASTKGP | YDTYRLADGVPSRFSG |
| | | | | SVFPLAPSSKSTSGGTAALGCLVKDYFPEPVT | SGSGTDYTLTISSLQP |
| | | | | VSWNSGALTSGVHTFPAVLQSSGLYSLSSVVT | EDFASYYCQQYYKFPF |
| | | | | VPSSSLGTQTYICNVNHKPSNTKVDKKVEPKS | TFGQGTKVEIKRTVAA |
| | | | | CDKTHTCAA | PSVFIFPPSDEQLKSG |
| | | | | | TASVVCLLNNFYPREA |
| | | | | | KVQWKVDNALQSGNSQ |
| | | | | | ESVTEQDSKDSTYSLS |
| | | | | | STLTLSKADYEKHKVY |
| | | | | | ACEVTHQGLSSPVTKS |
| | | | | | FNRGEC |
| CD40L | frexalimab | 254 | 255 | EVQLQESGPGLVKPSETLSLTCTVSGDSITNG | DIVMTQSPSFLSASVG |
| | | | | FWIWIRKPPGNKLEYMGYISYSGSTYYNPSLK | DRVTITCKASSNLGHA |
| | | | | SRISISRDTSKNQFSLKLSSVTAADTGVYYCAY | VAWYQQKPGKSPKLLI |
| | | | | RSYGRTPYYFDYWGQGTTLTVSSASTKGPSV | YSASNRYTGVPDRFSG |
| | | | | FPLAPSSKSTSGGTAALGCLVKDYFPEPVTVS | SGSGTDFTLTISSLQP |
| | | | | WNSGALTSGVHTFPAVLQSSGLYSLSSVVTVP | EDFADYFCQQYDDYPY |
| | | | | SSSLGTQTYICNVNHKPSNTKVDKKAEPKSCD | TFGGGTKLEIKRTVAA |
| | | | | KTHTCPPCPAPELLGGPSVFLFPPKPKDTLMIS | PSVFIFPPSDEQLKSG |
| | | | | RTPEVTCVVVDVSHRDPEVKFNWYVDGVEVH | TASVVCLLNNFYPREA |
| | | | | NAKTKPREEQYNSTYRVVSVLTVLHQDWLNG | KVQWKVDNALQSGNSQ |
| | | | | KEYKCAVSNKALPAPIEKTISKAKGQPREPQV | ESVTEQDSKDSTYSLS |
| | | | | YTLPPSRDELTKNQVSLTCLVKGFYPSDIAVE | STLTLSKADYEKHKVY |
| | | | | WESNGQPENNYKTTPPVLDSDGSFFLYSKLT | ACEVTHQGLSSPVTKS |
| | | | | VDKSRWQQGNVFSCSVMHEALHNHYTQKSL | FNRGEC |
| | | | | SLSPGK | |
| CD40L | letoli- | 256 | | EVQLLESGGGLVQPGGSLRLSCAASGFTFNW | |
| | zumab | | | ELMGWARQAPGKGLEWVSGIEGPGDVTYYA | |
| | | | | DSVKGRFTISRDNSKNTLYLQMNSLRAEDTAV | |
| | | | | YYCVKVGKDAKSDYRGQGTLVTVSSASTEPK | |
| | | | | SSDKTHTSPPSPAPELLGGSSVFLFPPKPKDT | |
| | | | | LMISRTPEVTCVVVDVSHEDPEVKFNWYVDG | |
| | | | | VEVHNAKTKPREEQYNSTYRVVSVLTVLHQD | |
| | | | | WLNGKEYKCKVSNKALPAPIEKTISKAKGQPR | |
| | | | | EPQVYTLPPSRDELTKNQVSLTCLVKGFYPSD | |
| | | | | IAVEWESNGQPENNYKTTPPVLDSDGSFFLYS | |
| | | | | KLTVDKSRWQQGNVFSCSVMHEALHNHYTQ | |
| | | | | KSLSLSPGK | |
| CD40L | ruplizumab | 257 | 258 | QVQLVQSGAEVVKPGASVKLSCKASGYIFTSY | DIVLTQSPATLSVSPG |
| | | | | YMYWVKQAPGQGLEWIGEINPSNGDTNFNEK | ERATISCRASQRVSSS |
| | | | | FKSKATLTVDKSASTAYMELSSLRSEDTAVYY | TYSYMHWYQQKPGQPP |
| | | | | CTRSDGRNDMDSWGQGTLVTVSSASTKGPS | KLLIKYASNLESGVPA |
| | | | | VFPLAPSSKSTSGGTAALGCLVKDYFPEPVTV | RFSGSGSGTDFTLTIS |
| | | | | SWNSGALTSGVHTFPAVLQSSGLYSLSSVVTV | SVEPEDFATYYCQHSW |
| | | | | PSSSLGTQTYICNVNHKPSNTKVDKKVEPKSC | EIPPTFGGGTKLEIKR |
| | | | | DKTHTCPPCPAPELLGGPSVFLFPPKPKDTLM | TVAAPSVFIFPPSDEQ |
| | | | | ISRTPEVTCVVVDVSHEDPEVKFNWYVDGVE | LKSGTASVVCLLNNFY |
| | | | | VHNAKTKPREEQYNSTYRVVSVLTVLHQDWL | PREAKVQWKVDNALQS |
| | | | | NGKEYKCKVSNKALPAPIEKTISKAKGQPREP | GNSQESVTEQDSKDST |
| | | | | QVYTLPPSRDELTKNQVSLTCLVKGFYPSDIA | YSLSSTLTLSKADYEK |
| | | | | VEWESNGQPENNYKTTPPVLDSDGSFFLYSK | HKVYACEVTHQGLSSP |
| | | | | LTVDKSRWQQGNVFSCSVMHEALHNHYTQK | VTKSFNRGEC |
| | | | | SLSLSPG | |
| CD5 | H65-CD5 | 259 | 260 | QIQLVQSGPELKKPGETVKISCKASGYTFTNY | DIKMTQSPSSMYASLG |
| | binding | | | GMNWVKQAPGKGLRWMGWINTHTGEPTYAD | ERVTITCKASQDINSY |
| | fragment | | | DFKGRFAFSLETSASTAYLQINNLKNEDTATYF | LSWFQQKPGKSPKTLI |
| | | | | CTRRGYDWYFDVWGAGTTVTVSS | YRANRLVDGVPSRFSG |
| | | | | | SGSGQDYSLTISSLDY |
| | | | | | EDMGIYYCQQYDESPW |
| | | | | | TFGGGTKLEIK |
| CD5 | 5D7-CD5 | 261 | 262 | QVTLKESGPGILKPSQTLSLTCSFSGFSLSTSG | NIVMTQSHKFMSTSVG |
| | binding | | | MGVGWIRQPSGKGLEWLAHIWWDDDVYYNP | DRVSIACKASQDVGTA |
| | fragment | | | SLKNQLTISKDASRDQVFLKITNLDTADTATYY | VAWYQQKPGQSPKLLI |
| | | | | CVRRRATGTGFDYWGQGTTLTVSS | YWTSTRHTGVPDRFTG |
| | | | | | SGSGTDFTLTISNVQS |
| | | | | | EDLADYFCQQYSSYP |
| CD5 | HE3-CD5 | 508 | 509 | EIQLVQSGGGLVKPGGSVRISCAASGYTFTNY | DIQMTQSPSSLSASVG |
| | binding | | | GMNWVRQAPGKGLEWMGWINTHTGEPTYAD | DRVTITCRASQDINSY |
| | fragment | | | SFKGRFTFSLDDSKNTAYLQINSLRAEDTAVYF | LSWFQQKPGKAPKTLI |
| | | | | CTRRGYDWYFDVWGQGTTVTVSS | YRANRLESGVPSRFSG |
| | | | | | SGSGTDYTLTISSLQY |
| | | | | | EDFGIYYCQQYDESPW |
| | | | | | TFGGGTKLEIK |
| CD56 | lorvotu- | 263 | 264 | QVQLVESGGGVVQPGRSLRLSCAASGFTFSS | DVVMTQSPLSLPVTLG |
| | zumab | | | FGMHWVRQAPGKGLEWVAYISSGSFTIYYAD | QPASISCRSSQIIIHS |
| | | | | SVKGRFTISRDNSKNTLYLQMNSLRAEDTAVY | DGNTYLEWFQQRPGQS |
| | | | | YCARMRKGYAMDYWGQGTLVTVSSASTKGP | PRRLIYKVSNRFSGVP |
| | | | | SVFPLAPSSKSTSGGTAALGCLVKDYFPEPVT | DRFSGSGSGTDFTLKI |
| | | | | VSWNSGALTSGVHTFPAVLQSSGLYSLSSVVT | SRVEAEDVGVYYCFQG |
| | | | | VPSSSLGTQTYICNVNHKPSNTKVDKKVEPKS | SHVPHTFGQGTKVEIK |
| | | | | CDKTHTCPPCPAPELLGGPSVFLFPPKPKDTL | RTVAAPSVFIFPPSDE |
| | | | | MISRTPEVTCVVVDVSHEDPEVKFNWYVDGV | QLKSGTASVVCLLNNF |
| | | | | EVHNAKTKPREEQYNSTYRVVSVLTVLHQDW | YPREAKVQWKVDNALQ |
| | | | | LNGKEYKCKVSNKALPAPIEKTISKAKGQPREP | SGNSQESVTEQDSKDS |
| | | | | QVYTLPPSRDELTKNQVSLTCLVKGFYPSDIA | TYSLSSTLTLSKADYE |
| | | | | VEWESNGQPENNYKTTPPVLDSDGSFFLYSK | KHKVYACEVTHQGLSS |
| | | | | LTVDKSRWQQGNVFSCSVMHEALHNHYTQK | PVTKSFNRGEC |
| | | | | SLSLSPGK | |
| CD7 | 3A1E | 265 | 266 | QVKLQESGGGLVKPGGSLKLSCAASGFTFSS | DIELTQSPAIMSASLG |
| | | | | YAMSWVRQTPEKRLEWVATISSGGSYTYYPD | EEITLTCSASSSVSYM |
| | | | | SVKGRFTISRDNAKNTLYLQMSSLRSEDTAMY | HWYQQKSGTSPKLLIY |
| | | | | YCARQDGYYPGWFANWGQGTTVTVSS | STSNLASGVPSRFSGS |
| | | | | | GSGTFYSLTISSVEAE |
| | | | | | DAADYYCHQWSSYTFG |
| | | | | | GGTKLEIKR |
| CD7 | 3A1F | 267 | 268 | QVQLQESGAELVKPGASVKLSCKASGYTFTS | DIELTQSPATLSVTPG |
| | | | | YWMHWVKQRPGQGLEWIGKINPSNGRTNYN | DSVSLSCRASQSISNN |
| | | | | EKFKSKATLTVDKSSSTAYMQLSSLTSEDSAV | LHWYQQKSHESPRLLI |
| | | | | YYCARGGVYYDLYYYALDYWGQGTTVTVSS | KSASQSISGIPSRFSG |
| | | | | | SGSGTDFTLSINSVET |
| | | | | | EDFGMYFCQQSNSWPY |
| | | | | | TFGGGTKLEIKR |
| CD7 | grisni- | 269 | 270 | QIQLVQSGPELKKPGETVKISCKASGYTFTNY | QAVVTQESALTTSPGE |
| | limab | | | GMNWVKQAPGKGLMWLGWINTYTGEPTYAD | TVTLTCRSSTGAVTTS |
| | | | | DFKGRFAFSLETSASTAYLQINNLKNEDTATYF | NYANWVQEKPDHLFTG |
| | | | | CARWAYFYGSSPYFFDYWGQGTTLTVSSAKT | LIGGTNNRAPGVPARF |
| | | | | TAPSVYPLAPVCGDTTGSSVTLGCLVKGYFPE | SGSLIGDKAALTITGA |
| | | | | PVTLTWNSGSLSSGVHTFPAVLQSDLYTLSSS | QTEDEAIYFCALWCSN |
| | | | | VTVTSSTWPSQSITCNVAHPASSTKVDKKIEP | HLVFGGGTKLTVLGQP |
| | | | | RGPTIKPCPPCKCPAPNLLGGPSVFIFPPKIKD | KSSPSVTLFPPSSEEL |
| | | | | VLMISLSPIVTCVVVDVSEDDPDVQISWFVNNV | ETNKATLVCTITDFYP |
| | | | | EVHTAQTQTHREDYNSTLRVVSALPIQHQDW | GVVTVDWKVDGTPVTQ |
| | | | | MSGKEFKCKVNNKDLPAPIERTISKPKGSVRA | GMETTQPSKQSNNKYM |
| | | | | PQVYVLPPPEEEMTKKQVTLTCMVTDFMPEDI | ASSYLTLTARAWERHS |
| | | | | YVEWTNNGKTELNYKNTEPVLDSDGSYFMYS | SYSCQVTHEGHTVEKS |
| | | | | KLRVEKKNWVERNSYSCSVVHEGLHNHHTTK | LSRADCS |
| | | | | SFSRTPGK | |
| CD7 | VHH-6- | 271 | | DVQLQESGGGLVQAGGSLRLSCAVSGYPYSS | |
| | camel | | | YCMGWFRQAPGKEREGVAAIDSDGRTRYAD | |
| | nanobody | | | SVKGRFTISQDNAKNTLYLQMNRMKPEDTAM | |
| | CD7 | | | YYCAARFGPMGCVDLSTLSFGHWGQGTQVT | |
| | binding | | | VSS | |
| | fragment | | | | |
| CD7 | WT1-CD7 | 272 | 273 | QIQLVQSGPELKKPGETVKISCKASGYTFTNY | QAVVTQESALTTSPGE |
| | binding | | | GMNWVKQAPGKGLMWLGWINTYTGEPTYAD | TVTLTCRSSTGAVTTS |
| | fragment | | | DFKGRFAFSLETSASTAYLQINNLKNEDTATYF | NYANWVQEKPDHLFTG |
| | | | | CARWAYFYGSSPYFFDYWGQGTTLTVSS | LIGGTNNRAPGVPARF |
| | | | | | SGSLIGDKAALTITGA |
| | | | | | QTEDEAIYFCALWCSN |
| | | | | | HLVFGGGTKLTVL |
| CD7 | TH-69-CD7 | 274 | 275 | QIQLVQSGPELKKPGETVKISCKASGYTFTNY | QAVVTQESALTTSPGE |
| | binding | | | GMNWVKQAPGKGLMWLGWINTYTGEPTYAD | TVTLTCRSSTGAVTTS |
| | fragment | | | DFKGRFAFSLETSASTAYLQINNLKNEDTATYF | NYANWVQEKPDHLFTG |
| | | | | CARWAYFYGSSPYFFDYWGQGTTLTVSS | LIGGTNNRAPGVPARF |
| | | | | | SGSLIGDKAALTITGA |
| | | | | | QTEDEAIYFCALWSNC |
| | | | | | HLVFGGGTKLTVL |
| CD70 | cusatu- | 276 | 277 | EVQLVESGGGLVQPGGSLRLSCAASGFTFSV | QAVVTQEPSLTVSPGG |
| | zumab | | | YYMNWVRQAPGKGLEWVSDINNEGGTTYYA | TVTLTCGLKSGSVTSD |
| | | | | DSVKGRFTISRDNSKNSLYLQMNSLRAEDTAV | NFPTWYQQTPGQAPRL |
| | | | | YYCARDAGYSNHVPIFDSWGQGTLVTVSSAS | LIYNTNTRHSGVPDRF |
| | | | | TKGPSVFPLAPSSKSTSGGTAALGCLVKDYFP | SGSILGNKAALTITGA |
| | | | | EPVTVSWNSGALTSGVHTFPAVLQSSGLYSLS | QADDEAEYFCALFISN |
| | | | | SVVTVPSSSLGTQTYICNVNHKPSNTKVDKKV | PSVEFGGGTQLTVLGQ |
| | | | | EPKSCDKTHTCPPCPAPELLGGPSVFLFPPKP | PKAAPSVTLFPPSSEE |
| | | | | KDTLMISRTPEVTCVVVDVSHEDPEVKFNWYV | LQANKATLVCLISDFY |
| | | | | DGVEVHNAKTKPREEQYNSTYRVVSVLTVLH | PGAVTVAWKADSSPVK |
| | | | | QDWLNGKEYKCKVSNKALPAPIEKTISKAKGQ | AGVETTTPSKQSNNKY |
| | | | | PREPQVYTLPPSRDELTKNQVSLTCLVKGFYP | AASSYLSLTPEQWKSH |
| | | | | SDIAVEWESNGQPENNYKTTPPVLDSDGSFFL | RSYSCQVTHEGSTVEK |
| | | | | YSKLTVDKSRWQQGNVFSCSVMHEALHNHYT | TVAPTECS |
| | | | | QKSLSLSPGK | |
| CD70 | vorsetu- | 278 | 279 | QVQLVQSGAEVKKPGASVKVSCKASGYTFTN | DIVMTQSPDSLAVSLG |
| | zumab | | | YGMNWVRQAPGQGLKWMGWINTYTGEPTYA | ERATINCRASKSVSTS |
| | | | | DAFKGRVTMTRDTSISTAYMELSRLRSDDTAV | GYSFMHWYQQKPGQPP |
| | | | | YYCARDYGDYGMDYWGQGTTVTVSSASTKG | KLLIYLASNLESGVPD |
| | | | | PSVFPLAPSSKSTSGGTAALGCLVKDYFPEPV | RFSGSGSGTDFTLTIS |
| | | | | TVSWNSGALTSGVHTFPAVLQSSGLYSLSSVV | SLQAEDVAVYYCQHSR |
| | | | | TVPSSSLGTQTYICNVNHKPSNTKVDKKVEPK | EVPWTFGQGTKVEIKR |
| | | | | SCDKTHTCPPCPAPELLGGPSVFLFPPKPKDT | TVAAPSVFIFPPSDEQ |
| | | | | LMISRTPEVTCVVVDVSHEDPEVKFNWYVDG | LKSGTASVVCLLNNFY |
| | | | | VEVHNAKTKPREEQYNSTYRVVSVLTVLHQD | PREAKVQWKVDNALQS |
| | | | | WLNGKEYKCKVSNKALPAPIEKTISKAKGQPR | GNSQESVTEQDSKDST |
| | | | | EPQVYTLPPSRDELTKNQVSLTCLVKGFYPSD | YSLSSTLTLSKADYEK |
| | | | | IAVEWESNGQPENNYKTTPPVLDSDGSFFLYS | HKVYACEVTHQGLSSP |
| | | | | KLTVDKSRWQQGNVFSCSVMHEALHNHYTQ | VTKSFNRGEC |
| | | | | KSLSLSPGK | |
| CD73 | dalutra- | 280 | 281 | EVQLVQSGAEVKKPGESLKISCKASGYAFSSS | DIQMTQSPSSLSASVG |
| | fusp | | | WINWVRQMPGKGLEWMGRIYPRAGDTNYAG | DRVTITCRASQDISNY |
| | | | | KFKDQVTISADKSISTAYLQWSSLKASDTAMY | LNWYQQKPGKAPKLLI |
| | | | | YCASLLDYSMDYWGQGTLVTVSSASTKGPSV | YYTSRLHSGVPSRFSG |
| | | | | FPLAPSSKSTSGGTAALGCLVKDYFPEPVTVS | SGSGTDFTFTISSLQP |
| | | | | WNSGALTSGVHTFPAVLQSSGLYSLSSVVTVP | EDIATYYCQQGNTLPL |
| | | | | SSSLGTQTYICNVNHKPSNTKVDKRVEPKSCD | TFGQGTKVEIKRTVAA |
| | | | | KTHTCPPCPAPELLGGPSVFLFPPKPKDTLMIS | PSVFIFPPSDEQLKSG |
| | | | | RTPEVTCVVVDVSHEDPEVKFNWYVDGVEVH | TASVVCLLNNFYPREA |
| | | | | NAKTKPREEQYASTYRVVSVLTVLHQDWLNG | KVQWKVDNALQSGNSQ |
| | | | | KEYKCKVSNKALPAPIEKTISKAKGQPREPQV | ESVTEQDSKDSTYSLS |
| | | | | YTLPPSREEMTKNQVSLTCLVKGFYPSDIAVE | STLTLSKADYEKHKVY |
| | | | | WESNGQPENNYKTTPPVLDSDGSFFLYSKLT | ACEVTHQGLSSPVTKS |
| | | | | VDKSRWQQGNVFSCSVMHEALHNHYTQKSL | FNRGEC |
| | | | | SLSPGGGGGSGGGGSGGGGSGGGGSGIPP | |
| | | | | HVQKSVNNDMIVTDNNGAVKFPQLCKFCDVR | |
| | | | | FSTCDNQKSCMSNCSITSICEKPQEVCVAVW | |
| | | | | RKNDENITLETVCHDPKLPYHDFILEDAASPKC | |
| | | | | IMKEKKKPGETFFMCSCSSDECNDNIIFSEEYN | |
| | | | | TSNPD | |
| CD73 | dresbuxe- | 282 | 283 | QVQLVQSGAEVVKPGASVKVSCKASGYSFTG | DIVMTQSPSSLAVSVG |
| | limab | | | YTMNWVRQAPGQNLEWIGLINPYNAGTSYNQ | ERVTISCKSSQSLLNS |
| | | | | KFQGKVTLTVDKSTSTAYMELSSLRSEDTAVY | SNQKNYLAWYQQKPGQ |
| | | | | YCARSEYRYGGDYFDYWGQGTTLTVSSASTK | APKLLIYFASTRESGV |
| | | | | GPSVFPLAPSSKSTSGGTAALGCLVKDYFPEP | PDRFSGSGSGTDFTLT |
| | | | | VTVSWNSGALTSGVHTFPAVLQSSGLYSLSSV | ISSLQAEDVAVYYCQQ |
| | | | | VTVPSSSLGTQTYICNVNHKPSNTKVDKKVEP | HYDTPYTFGGGTKLEI |
| | | | | KSCDKTHTCPPCPAPEAAGGPSVFLFPPKPK | KRTVAAPSVFIFPPSD |
| | | | | DTLMISRTPEVTCVVVDVSHEDPEVKFNWYVD | EQLKSGTASVVCLLNN |
| | | | | GVEVHNAKTKPREEQYNSTYRVVSVLTVLHQ | FYPREAKVQWKVDNAL |
| | | | | DWLNGKEYKCKVSNKALPAPIEKTISKAKGQP | QSGNSQESVTEQDSKD |
| | | | | REPQVYTLPPSRDELTKNQVSLTCLVKGFYPS | STYSLSSTLTLSKADY |
| | | | | DIAVEWESNGQPENNYKTTPPVLDSDGSFFLY | EKHKVYACEVTHQGLS |
| | | | | SKLTVDKSRWQQGNVFSCSVMHEALHNHYT | SPVTKSFNRGEC |
| | | | | QKSLSLSPGK | |
| CD73 | mupado- | 284 | 285 | QVQLVQSGAEVEKPGASVKVSCKASGYTFTS | EIVLTQSPATLSLSPG |
| | limab | | | YWITWVRQAPGQGLEWMGDIYPGSGNTNYN | ERATLSCRASKNVSTS |
| | | | | EKFKTRVTITADKSTSTAYMELSSLRSEDTAVY | GYSYMHWYQQKPGQAP |
| | | | | YCAKEGGLTTEDYALDYWGQGTLVTVSSAST | RLLIYLASNLESGIPP |
| | | | | KGPSVFPLAPSSKSTSGGTAALGCLVKDYFPE | RFSGSGYGTDFTLTIN |
| | | | | PVTVSWNSGALTSGVHTFPAVLQSSGLYSLSS | NIESEDAAYYFCQHSR |
| | | | | VVTVPSSSLGTQTYICNVNHKPSNTKVDKKVE | ELPFTFGQGTKVEIKR |
| | | | | PKSCDKTHTCPPCPAPELLGGPSVFLFPPKPK | TVAAPSVFIFPPSDEQ |
| | | | | DTLMISRTPEVTCVVVDVSHEDPEVKFNWYVD | LKSGTASVVCLLNNFY |
| | | | | GVEVHNAKTKPREEQYQSTYRVVSVLTVLHQ | PREAKVQWKVDNALQS |
| | | | | DWLNGKEYKCKVSNKALPAPIEKTISKAKGQP | GNSQESVTEQDSKDST |
| | | | | REPQVYTLPPSRDELTKNQVSLTCLVKGFYPS | YSLSSTLTLSKADYEK |
| | | | | DIAVEWESNGQPENNYKTTPPVLDSDGSFFLY | HKVYACEVTHQGLSSP |
| | | | | SKLTVDKSRWQQGNVFSCSVMHEALHNHYT | VTKSFNRGEC |
| | | | | QKSLSLSPGK | |
| CD73 | oleclumab | 286 | 287 | EVQLLESGGGLVQPGGSLRLSCAASGFTFSS | QSVLTQPPSASGTPGQ |
| | | | | YAYSWVRQAPGKGLEWVSAISGSGGRTYYAD | RVTISCSGSLSNIGRN |
| | | | | SVKGRFTISRDNSKNTLYLQMNSLRAEDTAVY | PVNWYQQLPGTAPKLL |
| | | | | YCARLGYGRVDEWGRGTLVTVSSASTKGPSV | IYLDNLRLSGVPDRFS |
| | | | | FPLAPSSKSTSGGTAALGCLVKDYFPEPVTVS | GSKSGTSASLAISGLQ |
| | | | | WNSGALTSGVHTFPAVLQSSGLYSLSSVVTVP | SEDEADYYCATWDDSH |
| | | | | SSSLGTQTYICNVNHKPSNTKVDKRVEPKSCD | PGWTFGGGTKLTVLGQ |
| | | | | KTHTCPPCPAPEFEGGPSVFLFPPKPKDTLMI | PKAAPSVTLFPPSSEE |
| | | | | SRTPEVTCVVVDVSHEDPEVKFNWYVDGVEV | LQANKATLVCLISDFY |
| | | | | HNAKTKPREEQYNSTYRVVSVLTVLHQDWLN | PGAVTVAWKADSSPVK |
| | | | | GKEYKCKVSNKALPASIEKTISKAKGQPREPQ | AGVETTTPSKQSNNKY |
| | | | | VYTLPPSREEMTKNQVSLTCLVKGFYPSDIAV | AASSYLSLTPEQWKSH |
| | | | | EWESNGQPENNYKTTPPVLDSDGSFFLYSKL | RSYSCQVTHEGSTVEK |
| | | | | TVDKSRWQQGNVFSCSVMHEALHNHYTQKS | TVAPTECS |
| | | | | LSLSPGK | |
| CD73 | uliled- | 288 | 289 | EVQLQESGPGLVKPSETLSLTCAVSGYSITSG | EIVLSQSPATLSLSPG |
| | limab | | | YYWNWIRQPPGKKLEWMGYINYGGSNGYNP | ERATLSCRASSRVNYM |
| | | | | SLKSRITISRDTSKNQFSLKLSSVTAADTAVYY | HWYQQKPGQSPRPWIS |
| | | | | CARDYDAYYEALDDWGQGTTVTVSSASTKGP | ATSNLASGVPARFSGS |
| | | | | SVFPLAPSSKSTSGGTAALGCLVKDYFPEPVT | GSGTSYTLTISSLEPE |
| | | | | VSWNSGALTSGVHTFPAVLQSSGLYSLSSVVT | DFAVYYCQQWSSNPPT |
| | | | | VPSSSLGTQTYICNVNHKPSNTKVDKKVEPKS | FGGGTKVEIKRTVAAP |
| | | | | CDKTHTCPPCPAPELLGGPSVFLFPPKPKDTL | SVFIFPPSDEQLKSGT |
| | | | | MISRTPEVTCVVVDVSHEDPEVKFNWYVDGV | ASVVCLLNNFYPREAK |
| | | | | EVHNAKTKPREEQYASTYRVVSVLTVLHQDW | VQWKVDNALQSGNSQE |
| | | | | LNGKEYKCKVSNKALPAPIEKTISKAKGQPREP | SVTEQDSKDSTYSLSS |
| | | | | QVYTLPPSREEMTKNQVSLTCLVKGFYPSDIA | TLTLSKADYEKHKVYA |
| | | | | VEWESNGQPENNYKTTPPVLDSDGSFFLYSK | CEVTHQGLSSPVTKSF |
| | | | | LTVDKSRWQQGNVFSCSVMHEALHNHYTQK | NRGEC |
| | | | | SLSLSPGK | |
| CD8 | 17D8-CD8 | 290 | 291 | QVQLVESGGDVVQPGGSLRLSCAASGVAFSN | EIVLTQSPATLSLSPG |
| | binding | | | YGMHWVRQAPGKGLEWVAVIWYDGSNKYYA | ERATLSCRASQSVSSY |
| | fragment | | | DSVKGRFTISRDNSKNMLYLQMNSLRAEDTA | LAWYQQKPGQAPRLII |
| | | | | MYYCARNDDYWGQGTLVTVSS | YDASNRATGIPARFSG |
| | | | | | SGSGTDFTLTISSLEP |
| | | | | | EDFAVYYCQQRSNWPL |
| | | | | | TFGGGTKVEIK |
| CD8 | IAB22M- | 292 | 293 | EVQLVESGGGLVQPGGSLRLSCAASGFNIKDT | DVQITQSPSSLSASVG |
| | CD8 | | | YIHFVRQAPGKGLEWIGRIDPANDNTLYASKF | DRVTITCRTSRSISQY |
| | binding | | | QGKATISADTSKNTAYLQMNSLRAEDTAVYYC | LAWYQQKPGKVPKLLI |
| | fragment | | | GRGYGYYVFDHWGQGTLVTVSS | YSGSTLQSGVPSRFSG |
| | | | | | SGSGTDFTLTISSLQP |
| | | | | | EDVATYYCQQHNENPL |
| | | | | | TFGGGTKVEIK |
| CD8 | OKT8 | 294 | 295 | EVQLVESGGGLVQPGGSLRLSCAASGFNIKDT | QVQLVQSGAEVKKPGA |
| | | | | YIHWVRQAPGKGLEWVARIDPANDNTLYASKF | TVKISCKVSGFNIKDT |
| | | | | QGRATISADTSKNTAYLQMNSLRAEDTAVYYC | YIHWVQQAPGKGLEWM |
| | | | | GRGYGYYVFDHWGQGTLVTVSS | GRIDPANDNTLYASKF |
| | | | | | QGRVTITADTSTDTAY |
| | | | | | MELSSLRSEDTAVYYC |
| | | | | | ARGYGYYVFDHWGQGT |
| | | | | | LVTVSS |
| CD8 | 3B5 | 296 | 297 | QVQLQQPGAELVRPGTSVKLSCRASGYTFTS | QIVLTQSPAIMSASPG |
| | | | | YWMHWVKQRPGQGLEWIGVIDPSDSYTNYN | EKVTMTCSASSRVSYV |
| | | | | QKFRGKATLTVDTSSSTAYMQLSSLTSEDSAV | HWYQQKSGTSPKRWLY |
| | | | | YYCARPYYGSHWFFDVWGTGTTVTVSS | DTSNLASGVPARFGGN |
| | | | | | GSGTSYSLTISSMEAE |
| | | | | | DAATYYCQQWSTNPPT |
| | | | | | FGGGTKLEIK |
| CTLA4 | Cadoni- | 298 | | EVQLVESGGGLVQPGGSLRLSCAASGFAFSS | |
| | limab- | | | YDMSWVRQAPGKGLDWVATISGGGRYTYYP | |
| | anti-PD1 | | | DSVKGRFTISRDNSKNNLYLQMNSLRAEDTAL | |
| | (heavy | | | YYCANRYGEAWFAYWGQGTLVTVSSASTKG | |
| | and | | | PSVFPLAPSSKSTSGGTAALGCLVKDYFPEPV | |
| | light | | | TVSWNSGALTSGVHTFPAVLQSSGLYSLSSVV | |
| | chain) | | | TVPSSSLGTQTYICNVNHKPSNTKVDKKVEPK | |
| | and | | | SCDKTHTCPPCPAPEAAGAPSVFLFPPKPKDT | |
| | anti- | | | LMISRTPEVTCVVVDVSHEDPEVKFNWYVDG | |
| | CTLA4 | | | VEVHNAKTKPREEQYNSTYRVVSVLTVLHQD | |
| | (heavy | | | WLNGKEYKCKVSNKALPAPIEKTISKAKGQPR | |
| | chain | | | EPQVYTLPPSRDELTKNQVSLTCLVKGFYPSD | |
| | only) | | | IAVEWESNGQPENNYKTTPPVLDSDGSFFLYS | |
| | | | | KLTVDKSRWQQGNVFSCSVMHEALHNHYTQ | |
| | | | | KSLSLSPGKGGGGSGGGGSGGGGSGGGGS | |
| | | | | QVQLVESGAEVKKPGASVKVSCKASGYSFTG | |
| | | | | YTMNWVRQAPGQCLEWIGLINPYNNITNYAQK | |
| | | | | FQGRVTFTVDTSISTAYMELSRLRSDDTGVYF | |
| | | | | CARLDYRSYWGQGTLVTVSAGGGGSGGGGS | |
| | | | | GGGGSGGGGSQAVVTQEPSLTVSPGGTVTL | |
| | | | | TCGSSTGAVTTSNFPNWVQQKPGQAPRSLIG | |
| | | | | GTNNKASWTPARFSGSLLGGKAALTISGAQP | |
| | | | | EDEAEYYCALWYSNHWVFGCGTKLTVLR | |
| CTLA4 | Erfonri- | 299 | | QVQLVESGGGLVQPGGSLRLSCAASGKMSS | |
| | limab- | | | RRCMAWFRQAPGKERERVAKLLTTSGSTYLA | |
| | bispecific | | | DSVKGRFTISRDNSKNTVYLQMNSLRAEDTAV | |
| | anti-CD274 | | | YYCAADSFEDPTCTLVTSSGAFQYWGQGTLV | |
| | and | | | TVSSGAPQVQLVESGGGLVQPGGSLRLSCAA | |
| | anti-CTLA4 | | | SGYIYSAYCMGWFRQAPGKGLEGVAAIYIGG | |
| | antibody | | | GSTYYADSVKGRFTISRDNSKNTLYLQMNSLR | |
| | | | | AEDTAVYYCAADVIPTETCLGGSWSGPFGYW | |
| | | | | GQGTLVTVSSGSEPKSSDKTHTCPPCPAPELL | |
| | | | | GGPSVFLFPPKPKDTLMISRTPEVTCVVVDVS | |
| | | | | HEDPEVKFNWYVDGVEVHNAKTKPREEQYNS | |
| | | | | TYRVVSVLTVLHQDWLNGKEYKCKVSNKALP | |
| | | | | APIEKTISKAKGQPREPQVYTLPPSRDELTKNQ | |
| | | | | VSLTCLVKGFYPSDIAVEWESNGQPENNYKTT | |
| | | | | PPVLDSDGSFFLYSKLTVDKSRWQQGNVFSC | |
| | | | | SVMHEALHNHYTQKSLSLSPGK | |
| CTLA4 | Loriger- | 300 | 301 | EIVLTQSPATLSLSPGERATLSCRASESVDNY | EIVLTQSPGTLSLSPG |
| | limab- | | | GMSFMNWFQQKPGQPPKLLIHAASNQGSGV | ERATLSCRASQSVSSS |
| | bispecific | | | PSRFSGSGSGTDFTLTISSLEPEDFAVYFCQQ | FLAWYQQKPGQAPRLL |
| | anti-PD-1 | | | SKEVPYTFGGGTKVEIKGGGSGGGGQVQLVE | IYGASSRATGIPDRFS |
| | and | | | SGGGVVQPGRSLRLSCAASGFTFSSYTMHW | GSGSGTDFTLTISRLE |
| | anti-CTLA4 | | | VRQAPGKGLEWVTFISYDGSNKHYADSVKGR | PEDFAVYYCQQYGSSP |
| | antibody | | | FTVSRDNSKNTLYLQMNSLRAEDTAIYYCART | WTFGQGTKVEIKGGGS |
| | | | | GWLGPFDYWGQGTLVTVSSGGCGGGEVAAC | GGGGQVQLVQSGAEVK |
| | | | | EKEVAALEKEVAALEKEVAALEKESKYGPPCP | KPGASVKVSCKASGYS |
| | | | | PCPAPEFLGGPSVFLFPPKPKDTLYITREPEVT | FTSYWMNWVRQAPGQG |
| | | | | CVVVDVSQEDPEVQFNWYVDGVEVHNAKTK | LEWIGVIHPSDSETWL |
| | | | | PREEQFNSTYRVVSVLTVLHQDWLNGKEYKC | DQKFKDRVTITVDKST |
| | | | | KVSNKGLPSSIEKTISKAKGQPREPQVYTLPPS | STAYMELSSLRSEDTA |
| | | | | QEEMTKNQVSLTCLVKGFYPSDIAVEWESNG | VYYCAREHYGTSPFAY |
| | | | | QPENNYKTTPPVLDSDGSFFLYSRLTVDKSR | WGQGTLVTVSSGGGGG |
| | | | | WQEGNVFSCSVMHEALHNHYTQKSLSLSLG | GKVAACKEKVAALKEK |
| | | | | | VAALKEKVAALKE |
| CTLA4 | vudalimab | 302 | 303 | EVQLVESGGGLVKPGGSLRLSCAASGFTFSS | EIVLTQSPGTLSLSPG |
| | | | | YTMHWVRQAPGKGLEWVSFISYDGNNKYYA | ERATLSCRASQSVSSS |
| | | | | DSVKGRFTISRDNAKNSLYLQMNSLRAEDTAV | YLAWYQQKPGQAPRLL |
| | | | | YYCARTGWLGPFDYWGQGTLVTVSSASTKG | IYGAFSRATGIPDRFS |
| | | | | PSVFPLAPSSKSTSGGTAALGCLVKDYFPEPV | GSGSGTDFTLTISRLE |
| | | | | TVSWNSGALTSGVHTFPAVLQSSGLYSLSSVV | PEDFAVYYCQQYGSSP |
| | | | | TVPSSSLGTQTYICNVNHKPSDTKVDKKVEPK | WTFGQGTKVEIKRTVA |
| | | | | SCDKTHTCPPCPAPPVAGPSVFLFPPKPKDTL | APSVFIFPPSDEQLKS |
| | | | | MISRTPEVTCVVVDVKHEDPEVKFNWYVDGV | GTASVVCLLNNFYPRE |
| | | | | EVHNAKTKPREEEYNSTYRVVSVLTVLHQDW | AKVQWKVDNALQSGNS |
| | | | | LNGKEYKCKVSNKALPAPIEKTISKAKGQPREP | QESVTEQDSKDSTYSL |
| | | | | QVYTLPPSREEMTKNQVSLTCDVSGFYPSDIA | SSTLTLSKADYEKHKV |
| | | | | VEWESDGQPENNYKTTPPVLDSDGSFFLYSK | YACEVTHQGLSSPVTK |
| | | | | LTVDKSRWEQGDVFSCSVLHEALHSHYTQKS | SFNRGEC |
| | | | | LSLSPGK | |
| CTLA-4 | bavuna- | 304 | 305 | EVQLVESGGGLVQPGGSLRLSCAASGFTFDD | DIVLTQSPSSLSASVG |
| | limab | | | AWMSWVRQAPGKGLEWVAEISTKANNHATY | DRVTITCRASQSVDYD |
| | | | | YAESVKGRFTISRDDSKSSVYLQMNSLRAEDT | GDSYMNWYQQKPGKPP |
| | | | | AVYYCTRLATWDWYFDVWGQGTTVTVSSAS | KLLIYAASELESGIPA |
| | | | | TKGPSVFPLAPSSKSTSGGTAALGCLVKDYFP | RFSGSGSGTDFTLTIS |
| | | | | EPVTVSWNSGALTSGVHTFPAVLQSSGLYSLS | SLQPEDFATYYCQQSN |
| | | | | SVVTVPSSSLGTQTYICNVNHKPSDTKVDKKV | EDPFTFGSGTKLEIKR |
| | | | | EPKSCDKTHTCPPCPAPPVAGPSVFLFPPKPK | TVAAPSVFIFPPSDEQ |
| | | | | DTLMISRTPEVTCVVVDVKHEDPEVKFNWYVD | LKSGTASVVCLLNNFY |
| | | | | GVEVHNAKTKPREEEYNSTYRVVSVLTVLHQ | PREAKVQWKVDNALQS |
| | | | | DWLNGKEYKCKVSNKALPAPIEKTISKAKGQP | GNSQESVTEQDSKDST |
| | | | | REPQVYTLPPSREEMTKNQVSLTCDVSGFYP | YSLSSTLTLSKADYEK |
| | | | | SDIAVEWESDGQPENNYKTTPPVLDSDGSFFL | HKVYACEVTHQGLSSP |
| | | | | YSKLTVDKSRWEQGDVFSCSVLHEALHSHYT | VTKSFNRGEC |
| | | | | QKSLSLSPGK | |
| CTLA-4 | botensi- | 306 | 307 | EVQLVESGGGLVKPGGSLRLSCAASGFTFSS | EIVLTQSPGTLSLSPG |
| | limab | | | YSMNWVRQAPGKGLEWVSSISSSSSYIYYAE | ERATLSCRASQSVSRY |
| | | | | SVKGRFTISRDNAKNSLYLQMNSLRAEDTAVY | LGWYQQKPGQAPRLLI |
| | | | | YCARVGLFGPFDIWGQGTLVTVSSASTKGPS | YGASTRATGIPDRFSG |
| | | | | VFPLAPSSKSTSGGTAALGCLVKDYFPEPVTV | SGSGTDFTLTITRLEP |
| | | | | SWNSGALTSGVHTFPAVLQSSGLYSLSSVVTV | EDFAVYYCQQYGSSPW |
| | | | | PSSSLGTQTYICNVNHKPSNTKVDKRVEPKSC | TFGQGTKVEIKRTVAA |
| | | | | DKTHTCPPCPAPELLGGPDVFLFPPKPKDTLM | PSVFIFPPSDEQLKSG |
| | | | | ISRTPEVTCVVVDVSHEDPEVKFNWYVDGVE | TASVVCLLNNFYPREA |
| | | | | VHNAKTKPREEQYNSTYRVVSVLTVLHQDWL | KVQWKVDNALQSGNSQ |
| | | | | NGKEYKCKVSNKALPLPEEKTISKAKGQPREP | ESVTEQDSKDSTYSLS |
| | | | | QVYTLPPSREEMTKNQVSLTCLVKGFYPSDIA | STLTLSKADYEKHKVY |
| | | | | VEWESNGQPENNYKTTPPVLDSDGSFFLYSK | ACEVTHQGLSSPVTKS |
| | | | | LTVDKSRWQQGNVFSCSVMHEALHNHYTQK | FNRGEC |
| | | | | SLSLSPG | |
| CTLA-4 | Cadoni- | 308 | 309 | EVQLVESGGGLVQPGGSLRLSCAASGFAFSS | DIQMTQSPSSMSASVG |
| | limab- | | | YDMSWVRQAPGKGLDWVATISGGGRYTYYP | DRVTFTCRASQDINTY |
| | bispecific | | | DSVKGRFTISRDNSKNNLYLQMNSLRAEDTAL | LSWFQQKPGKSPKTLI |
| | anti-PD1 | | | YYCANRYGEAWFAYWGQGTLVTVSSASTKG | YRANRLVSGVPSRFSG |
| | and | | | PSVFPLAPSSKSTSGGTAALGCLVKDYFPEPV | SGSGQDYTLTISSLQP |
| | anti- | | | TVSWNSGALTSGVHTFPAVLQSSGLYSLSSVV | EDMATYYCLQYDEFPL |
| | CTLA-4 | | | TVPSSSLGTQTYICNVNHKPSNTKVDKKVEPK | TFGAGTKLELKRTVAA |
| | antibody | | | SCDKTHTCPPCPAPEAAGAPSVFLFPPKPKDT | PSVFIFPPSDEQLKSG |
| | | | | LMISRTPEVTCVVVDVSHEDPEVKFNWYVDG | TASVVCLLNNFYPREA |
| | | | | VEVHNAKTKPREEQYNSTYRVVSVLTVLHQD | KVQWKVDNALQSGNSQ |
| | | | | WLNGKEYKCKVSNKALPAPIEKTISKAKGQPR | ESVTEQDSKDSTYSLS |
| | | | | EPQVYTLPPSRDELTKNQVSLTCLVKGFYPSD | STLTLSKADYEKHKVY |
| | | | | IAVEWESNGQPENNYKTTPPVLDSDGSFFLYS | ACEVTHQGLSSPVTKS |
| | | | | KLTVDKSRWQQGNVFSCSVMHEALHNHYTQ | FNRGEC |
| | | | | KSLSLSPGKGGGGSGGGGSGGGGSGGGGS | |
| | | | | QVQLVESGAEVKKPGASVKVSCKASGYSFTG | |
| | | | | YTMNWVRQAPGQCLEWIGLINPYNNITNYAQK | |
| | | | | FQGRVTFTVDTSISTAYMELSRLRSDDTGVYF | |
| | | | | CARLDYRSYWGQGTLVTVSAGGGGSGGGGS | |
| | | | | GGGGSGGGGSQAVVTQEPSLTVSPGGTVTL | |
| | | | | TCGSSTGAVTTSNFPNWVQQKPGQAPRSLIG | |
| | | | | GTNNKASWTPARFSGSLLGGKAALTISGAQP | |
| | | | | EDEAEYYCALWYSNHWVFGCGTKLTVLR | |
| CTLA-4 | ipilimumab | 310 | 311 | QVQLVESGGGVVQPGRSLRLSCAASGFTFSS | EIVLTQSPGTLSLSPG |
| | | | | YTMHWVRQAPGKGLEWVTFISYDGNNKYYAD | ERATLSCRASQSVGSS |
| | | | | SVKGRFTISRDNSKNTLYLQMNSLRAEDTAIYY | YLAWYQQKPGQAPRLL |
| | | | | CARTGWLGPFDYWGQGTLVTVSSASTKGPS | IYGAFSRATGIPDRFS |
| | | | | VFPLAPSSKSTSGGTAALGCLVKDYFPEPVTV | GSGSGTDFTLTISRLE |
| | | | | SWNSGALTSGVHTFPAVLQSSGLYSLSSVVTV | PEDFAVYYCQQYGSSP |
| | | | | PSSSLGTQTYICNVNHKPSNTKVDKRVEPKSC | WTFGQGTKVEIKRTVA |
| | | | | DKTHTCPPCPAPELLGGPSVFLFPPKPKDTLM | APSVFIFPPSDEQLKS |
| | | | | ISRTPEVTCVVVDVSHEDPEVKFNWYVDGVE | GTASVVCLLNNFYPRE |
| | | | | VHNAKTKPREEQYNSTYRVVSVLTVLHQDWL | AKVQWKVDNALQSGNS |
| | | | | NGKEYKCKVSNKALPAPIEKTISKAKGQPREP | QESVTEQDSKDSTYSL |
| | | | | QVYTLPPSRDELTKNQVSLTCLVKGFYPSDIA | SSTLTLSKADYEKHKV |
| | | | | VEWESNGQPENNYKTTPPVLDSDGSFFLYSK | YACEVTHQGLSSPVTK |
| | | | | LTVDKSRWQQGNVFSCSVMHEALHNHYTQK | SFNRGEC |
| | | | | SLSLSPGK | |
| CTLA-4 | JMW-3B3- | 312 | 313 | EVKLVNSGGGLVQPGNSLRLSCATSGFTFTDF | QAVVTQESAFTTSPGG |
| | CTLA-4 | | | YMSWVRQPPGKALEWLGFVRNRANGYTTEY | TVILTCRSSTGAVTTN |
| | binding | | | SVSVKGRFIISRDNFQSTLFLQMNTLRAEDSG | NYANWVQEKPDHLFTG |
| | fragment | | | TYYCVRGPGDTADYTMDYWGQGTSVTVSS | LIGGTSNRAPGVPVRF |
| | | | | | SGSLIGDKAALTITGA |
| | | | | | QTEDDGMYFCALWYTT |
| | | | | | HFVFGGGTKVT |
| CTLA-4 | nurulimab | 314 | 315 | EVQLVESGGGVVQPGRSLRLSCAASGFTFSS | EIVLTQSPGTLSLSPG |
| | | | | YTMHWVRQAPGKGLEWVTFISYDGNNKYYAD | ERATLSCRASQSVGSS |
| | | | | SVKGRFTISRDNSKNTLYLQMNSLRAEDTAIYY | YLAWYQQKPGQAPRLL |
| | | | | CARTGWLGPFDYWGQGTLVTVSSASTKGPS | IYGAFSRATGIPDRFS |
| | | | | VFPLAPSSKSTSGGTAALGCLVKDYFPEPVTV | GSGSGTDFTLTISRLE |
| | | | | SWNSGALTSGVHTFPAVLQSSGLYSLSSVVTV | PEDFAVYYCQQYGSSP |
| | | | | PSSSLGTQTYICNVNHKPSNTKVDKRVEPKSC | WTFGQGTKVEIKRTVA |
| | | | | DKTHTCPPCPAPELLGGPSVFLFPPKPKDTLM | APSVFIFPPSDEQLKS |
| | | | | ISRTPEVTCVVVDVSHEDPEVKFNWYVDGVE | GTASVVCLLNNFYPRE |
| | | | | VHNAKTKPREEQYNSTYRVVSVLTVLHQDWL | AKVQWKVDNALQSGNS |
| | | | | NGKEYKCKVSNKALPAPIEKTISKAKGQPREP | QESVTEQDSKDSTYSL |
| | | | | QVYTLPPSRDELTKNQVSLTCLVKGFYPSDIA | SSTLTLSKADYEKHKV |
| | | | | VEWESNGQPENNYKTTPPVLDSDGSFFLYSK | YACEVTHQGLSSPVTK |
| | | | | LTVDKSRWQQGNVFSCSVMHEALHNHYTQK | SFNRGEC |
| | | | | SLSLSP | |
| CTLA-4 | quavon- | 316 | 317 | EVQLVESGGGLVQPGGSLRLSCAASGFTFSD | DIQMTQSPSSLSASVG |
| | limab | | | NWMNWVRQAPGKGLEWLAQIRNKPYNYETY | DRVTITCRTSENIYGG |
| | | | | YSASVKGRFTISRDDSKNSVYLQMNSLKTEDT | LNWYQRKPGKSPKLLI |
| | | | | GVYYCTAQFAYWGQGTLVTVSSASTKGPSVF | YGATNLASGVSSRFSG |
| | | | | PLAPSSKSTSGGTAALGCLVKDYFPEPVTVSW | SGSGTDYTLTISSLQP |
| | | | | NSGALTSGVHTFPAVLQSSGLYSLSSVVTVPS | EDVATYYCQNVLRSPF |
| | | | | SSLGTQTYICNVNHKPSNTKVDKKVEPKSCDK | TFGSGTKLEIKRTVAA |
| | | | | THTCPPCPAPELLGGPSVFLFPPKPKDTLMIS | PSVFIFPPSDEQLKSG |
| | | | | RTPEVTCVVVDVSHEDPEVKFNWYVDGVEVH | TASVVCLLNNFYPREA |
| | | | | NAKTKPREEQYNSTYRVVSVLTVLHQDWLNG | KVQWKVDNALQSGNSQ |
| | | | | KEYKCKVSNKALPAPIEKTISKAKGQPREPQV | ESVTEQDSKDSTYSLS |
| | | | | YTLPPSRDELTKNQVSLTCLVKGFYPSDIAVE | STLTLSKADYEKHKVY |
| | | | | WESNGQPENNYKTTPPVLDSDGSFFLYSKLT | ACEVTHQGLSSPVTKS |
| | | | | VDKSRWQQGNVFSCSVMHEALHNHYTQKSL | FNRGEC |
| | | | | SLSPGK | |
| CTLA-4 | tremeli- | 318 | 319 | QVQLVESGGGVVQPGRSLRLSCAASGFTFSS | DIQMTQSPSSLSASVG |
| | mumab | | | YGMHWVRQAPGKGLEWVAVIWYDGSNKYYA | DRVTITCRASQSINSY |
| | | | | DSVKGRFTISRDNSKNTLYLQMNSLRAEDTAV | LDWYQQKPGKAPKLLI |
| | | | | YYCARDPRGATLYYYYYGMDVWGQGTTVTV | YAASSLQSGVPSRFSG |
| | | | | SSASTKGPSVFPLAPCSRSTSESTAALGCLVK | SGSGTDFTLTISSLQP |
| | | | | DYFPEPVTVSWNSGALTSGVHTFPAVLQSSG | EDFATYYCQQYYSTPF |
| | | | | LYSLSSVVTVPSSNFGTQTYTCNVDHKPSNTK | TFGPGTKVEIKRTVAA |
| | | | | VDKTVERKCCVECPPCPAPPVAGPSVFLFPPK | PSVFIFPPSDEQLKSG |
| | | | | PKDTLMISRTPEVTCVVVDVSHEDPEVQFNW | TASVVCLLNNFYPREA |
| | | | | YVDGVEVHNAKTKPREEQFNSTFRVVSVLTVV | KVQWKVDNALQSGNSQ |
| | | | | HQDWLNGKEYKCKVSNKGLPAPIEKTISKTKG | ESVTEQDSKDSTYSLS |
| | | | | QPREPQVYTLPPSREEMTKNQVSLTCLVKGF | STLTLSKADYEKHKVY |
| | | | | YPSDIAVEWESNGQPENNYKTTPPMLDSDGS | ACEVTHQGLSSPVTKS |
| | | | | FFLYSKLTVDKSRWQQGNVFSCSVMHEALHN | FNRGEC |
| | | | | HYTQKSLSLSPGK | |
| CTLA-4 | tuvonra- | 320 | 321 | QVQLVESGGGVVEPGRSLRLSCAASGFTFSS | EIVLTQSPGTLSLSPG |
| | limab | | | YGMHWVRQAPGKGLEWVAVIWYKPSEKDYA | ERATLSCRASQSINSY |
| | | | | DSAKGRFTISRDNSKNTLYLQMNSLRAEDTAV | LAWYQQKPGQAPRPLI |
| | | | | YYCARGGLLGYFDYWGQGTLVTVSSASTKGP | YGVSSRATGIPDRFSG |
| | | | | SVFPLAPSSKSTSGGTAALGCLVDDYFPEPVT | SGSGTDFTLTISRLEP |
| | | | | VSWNSGALTSGVHTCPACLQSSGLYSLSSVV | EDFAVYYCQQYGRYPF |
| | | | | TVPSSSLGTQTYICNVNHKPSNTKVDKKVEPK | TFGPGTKVDIKRTVAA |
| | | | | SGDKTHTCPPCPAPELLGGPSVFLFPPKPKDT | PSVFIFPPSDEQLKSG |
| | | | | LMISKTPEVTCVVVDVSHEDPEVKFNWYVDG | TAKVVCLLNNFYPREA |
| | | | | VEVHNAKTKPREEQYNSTYRVVSVLTVLHQD | KVQWKVDNALQSGNSC |
| | | | | WLNGKEYKCKVSNKALPAPIEKTISKAKGQPR | ECVTEQDSKDSTYSLS |
| | | | | EPQVYTLPPSREEMTKNQVSLTCLVKGFYPSD | STLTLSKADYEKHKVY |
| | | | | IAVEWESNGQPENNYKTTPPVLRSDGSFFLYS | ACEVTHQGLSSPVTKS |
| | | | | ELTVDKSRWQQGNVFSCSVMHEALHNHYTQ | FNRGES |
| | | | | KSLSLSPGK | |
| CTLA-4 | VH5:VK4 | 322 | 323 | QVQLVQSGAELKKPGASVKVSCKASGYTFTS | EIVLTQSPATLSLSPG |
| | | | | YWINWIRQAPGQGLEWIGRIAPGSGTTYYNEV | ERATLSCSASSSISYM |
| | | | | FKGRVTITVDKSTSTAYMELSSLRSEDTAVYF | HWFQQRPGQSPRRWIY |
| | | | | CARGDYGSYWGQGTLVTVSS | DTSKLASGVPARFSGS |
| | | | | | GSGTDYTLTISSLEPE |
| | | | | | DFATYYCHQRTSYPLT |
| | | | | | FGQGTKLEIKR |
| CTLA-4 | zalifre- | 324 | 325 | EVQLVESGGGLVKPGGSLRLSCAASGFTFSS | EIVLTQSPGTLSLSPG |
| | limab | | | YSMNWVRQAPGKGLEWVSSISSSSSYIYYAD | ERATLSCRASQSVSRY |
| | | | | SVKGRFTISRDNAKNSLYLQMNSLRAEDTAVY | LGWYQQKPGQAPRLLI |
| | | | | YCARVGLMGPFDIWGQGTMVTVSSASTKGPS | YGASTRATGIPDRFSG |
| | | | | VFPLAPSSKSTSGGTAALGCLVKDYFPEPVTV | SGSGTDFTLTITRLEP |
| | | | | SWNSGALTSGVHTFPAVLQSSGLYSLSSVVTV | EDFAVYYCQQYGSSPW |
| | | | | PSSSLGTQTYICNVNHKPSNTKVDKRVEPKSC | TFGQGTKVEIKRTVAA |
| | | | | DKTHTCPPCPAPELLGGPSVFLFPPKPKDTLM | PSVFIFPPSDEQLKSG |
| | | | | ISRTPEVTCVVVDVSHEDPEVKFNWYVDGVE | TASVVCLLNNFYPREA |
| | | | | VHNAKTKPREEQYNSTYRVVSVLTVLHQDWL | KVQWKVDNALQSGNSQ |
| | | | | NGKEYKCKVSNKALPAPIEKTISKAKGQPREP | ESVTEQDSKDSTYSLS |
| | | | | QVYTLPPSREEMTKNQVSLTCLVKGFYPSDIA | STLTLSKADYEKHKVY |
| | | | | VEWESNGQPENNYKTTPPVLDSDGSFFLYSK | ACEVTHQGLSSPVTKS |
| | | | | LTVDKSRWQQGNVFSCSVMHEALHNHYTQK | FNRGEC |
| | | | | SLSLSPGK | |
| GD2 | dinutux- | 326 | 327 | EVQLLQSGPELEKPGASVMISCKASGSSFTGY | EIVMTQSPATLSVSPG |
| | imab | | | NMNWVRQNIGKSLEWIGAIDPYYGGTSYNQK | ERATLSCRSSQSLVHR |
| | | | | FKGRATLTVDKSSSTAYMHLKSLTSEDSAVYY | NGNTYLHWYLQKPGQS |
| | | | | CVSGMEYWGQGTSVTVSSASTKGPSVFPLAP | PKLLIHKVSNRFSGVP |
| | | | | SSKSTSGGTAALGCLVKDYFPEPVTVSWNSG | DRFSGSGSGTDFTLKI |
| | | | | ALTSGVHTFPAVLQSSGLYSLSSVVTVPSSSL | SRVEAEDLGVYFCSQS |
| | | | | GTQTYICNVNHKPSNTKVDKRVEPKSCDKTHT | THVPPLTFGAGTKLEL |
| | | | | CPPCPAPELLGGPSVFLFPPKPKDTLMISRTP | KRTVAAPSVFIFPPSD |
| | | | | EVTCVVVDVSHEDPEVKFNWYVDGVEVHNAK | EQLKSGTASVVCLLNN |
| | | | | TKPREEQYNSTYRVVSVLTVLHQDWLNGKEY | FYPREAKVQWKVDNAL |
| | | | | KCKVSNKALPAPIEKTISKAKGQPREPQVYTLP | QSGNSQESVTEQDSKD |
| | | | | PSREEMTKNQVSLTCLVKGFYPSDIAVEWESN | STYSLSSTLTLSKADY |
| | | | | GQPENNYKTTPPVLDSDGSFFLYSKLTVDKSR | EKHKVYACEVTHQGLS |
| | | | | WQQGNVFSCSVMHEALHNHYTQKSLSLSPG | SPVTKSFNRGEC |
| | | | | K | |
| GD2 | lorukafusp | 328 | 329 | EVQLVQSGAEVEKPGASVKISCKASGSSFTGY | DVVMTQTPLSLPVTPG |
| | | | | NMNWVRQNIGKSLEWIGAIDPYYGGTSYNQK | EPASISCRSSQSLVHR |
| | | | | FKGRATLTVDKSTSTAYMHLKSLRSEDTAVYY | NGNTYLHWYLQKPGQS |
| | | | | CVSGMEYWGQGTSVTVSSASTKGPSVFPLAP | PKLLIHKVSNRFSGVP |
| | | | | SSKSTSGGTAALGCLVKDYFPEPVTVSWNSG | DRFSGSGSGTDFTLKI |
| | | | | ALTSGVHTFPAVLQSSGLYSLSSVVTVPSSSL | SRVEAEDLGVYFCSQS |
| | | | | GTQTYICNVNHKPSNTKVDKRVEPKSCDKTHT | THVPPLTFGAGTKLEL |
| | | | | CPPCPAPELLGGPSVFLFPPKPKDTLMISRTP | KRTVAAPSVFIFPPSD |
| | | | | EVTCVVVDVSHEDPEVKFNWYVDGVEVHNAK | EQLKSGTASVVCLLNN |
| | | | | TKPREEQYNSTYRVVSVLTVLHQDWLNGKEY | FYPREAKVQWKVDNAL |
| | | | | KCKVSNKALPAPIEKTISKAKGQPREPQVYTLP | QSGNSQESVTEQDSKD |
| | | | | PSREEMTKNQVSLTCLVKGFYPSDIAVEWESN | STYSLSSTLTLSKADY |
| | | | | GQPENNYKTTPPVLDSDGSFFLYSKLTVDKSR | EKHKVYACEVTHQGLS |
| | | | | WQQGNVFSCSVMHEALHNHYTQKSLSLSPG | SPVTKSFNRGEC |
| | | | | KAPTSSSTKKTQLQLEHLLLDLQMILNGINNYK | |
| | | | | NPKLTRMLTFKFYMPKKATELKHLQCLEEELK | |
| | | | | PLEEVLNLAQSKNFHLRPRDLISNINVIVLELK | |
| | | | | GSETTFMCEYADETATIVEFLNRWITFCQSIIS | |
| | | | | TLT | |
| GD2 | naxitamab | 330 | 331 | QVQLVESGPGVVQPGRSLRISCAVSGFSVTN | EIVMTQTPATLSVSAG |
| | | | | YGVHWVRQPPGKGLEWLGVIWAGGITNYNSA | ERVTITCKASQSVSND |
| | | | | FMSRLTISKDNSKNTVYLQMNSLRAEDTAMYY | VTWYQQKPGQAPRLLI |
| | | | | CASRGGHYGYALDYWGQGTLVTVSSASTKG | YSASNRYSGVPARFSG |
| | | | | PSVFPLAPSSKSTSGGTAALGCLVKDYFPEPV | SGYGTEFTFTISSVQS |
| | | | | TVSWNSGALTSGVHTFPAVLQSSGLYSLSSVV | EDFAVYFCQQDYSSFG |
| | | | | TVPSSSLGTQTYICNVNHKPSNTKVDKRVEPK | QGTKLEIKRTVAAPSV |
| | | | | SCDKTHTCPPCPAPELLGGPSVFLFPPKPKDT | FIFPPSDEQLKSGTAS |
| | | | | LMISRTPEVTCVVVDVSHEDPEVKFNWYVDG | VVCLLNNFYPREAKVQ |
| | | | | VEVHNAKTKPREEQYNSTYRVVSVLTVLHQD | WKVDNALQSGNSQESV |
| | | | | WLNGKEYKCKVSNKALPAPIEKTISKAKGQPR | TEQDSKDSTYSLSSTL |
| | | | | EPQVYTLPPSRDELTKNQVSLTCLVKGFYPSD | TLSKADYEKHKVYACE |
| | | | | IAVEWESNGQPENNYKTTPPVLDSDGSFFLYS | VTHQGLSSPVTKSFNR |
| | | | | KLTVDKSRWQQGNVFSCSVMHEALHNHYTQ | GEC |
| | | | | KSLSLSPGK | |
| GD2 | Nivatro | 332 | 333 | QVQLVESGPGVVQPGRSLRISCAVSGFSVTN | EIVMTQTPATLSVSAG |
| | tamab- | | | YGVHWVRQPPGKGLEWLGVIWAGGITNYNSA | ERVTITCKASQSVSND |
| | bispecific | | | FMSRLTISKDNSKNTVYLQMNSLRAEDTAMYY | VTWYQQKPGQAPRLLI |
| | anti-CD3E | | | CASRGGHYGYALDYWGQGTLVTVSSASTKG | YSASNRYSGVPARFSG |
| | and | | | PSVFPLAPSSKSTSGGTAALGCLVKDYFPEPV | SGYGTEFTFTISSVQS |
| | anti-GD2 | | | TVSWNSGALTSGVHTFPAVLQSSGLYSLSSVV | EDFAVYFCQQDYSSFG |
| | antibody | | | TVPSSSLGTQTYICNVNHKPSNTKVDKRVEPK | QGTKLEIKRTVAAPSV |
| | | | | SCDKTHTCPPCPAPELLGGPSVFLFPPKPKDT | FIFPPSDEQLKSGTAS |
| | | | | LMISRTPEVTCVVVDVSHEDPEVKFNWYVDG | VVCLLNNFYPREAKVQ |
| | | | | VEVHNAKTKPREEQYASTYRVVSVLTVLHQD | WKVDNALQSGNSQESV |
| | | | | WLNGKEYKCAVSNKALPAPIEKTISKAKGQPR | TEQDSKDSTYSLSSTL |
| | | | | EPQVYTLPPSRDELTKNQVSLTCLVKGFYPSD | TLSKADYEKHKVYACE |
| | | | | IAVEWESNGQPENNYKTTPPVLDSDGSFFLYS | VTHQGLSSPVTKSFNR |
| | | | | KLTVDKSRWQQGNVFSCSVMHEALHNHYTQ | GECTSGGGGSGGGGSG |
| | | | | KSLSLSPGK | GGGSQVQLVQSGGGVV |
| | | | | | QPGRSLRLSCKASGYT |
| | | | | | FTRYTMHWVRQAPGKC |
| | | | | | LEWIGYINPSRGYTNY |
| | | | | | NQKFKDRFTISRDNSK |
| | | | | | NTAFLQMDSLRPEDTG |
| | | | | | VYFCARYYDDHYSLDY |
| | | | | | WGQGTPVTVSSGGGGS |
| | | | | | GGGGSGGGGSGGGGSG |
| | | | | | GGGSGGGGSDIQMTQS |
| | | | | | PSSLSASVGDRVTITC |
| | | | | | SASSSVSYMNWYQQTP |
| | | | | | GKAPKRWIYDTSKLAS |
| | | | | | GVPSRFSGSGSGTDYT |
| | | | | | FTISSLQPEDIATYYC |
| | | | | | QQWSSNPFTFGCGTKL |
| | | | | | QITR |
| GITR | INCAGN0187 | 334 | 335 | QVQLVQSGAEVKKPGASVKVSCKGSGYTFTD | DIVMTQSPDSLAVSLG |
| | 6-GITR | | | YAMYWVRQAPGQGLEWIGVIRTYSGDVTYNQ | ERATINCKSSQSLLNS |
| | binding | | | KFKDRATMTVDKSISTAYMELSRLRSDDTAVY | GNQKNYLTWYQQKPGQ |
| | fragment | | | YCAKSGTVRGFAYWGQGTLVTVSS | PPKLLIYWASTRESGV |
| | | | | | PDRFSGSGSGTDFTLT |
| | | | | | ISSLQAEDVAVYHCQN |
| | | | | | DYSYPYTFGQGTKLEI |
| | | | | | K |
| GITR | ragifi- | 336 | 337 | QVQLVQSGAEVKKPGASVKVSCKGSGYTFTD | DIVMTQSPDSLAVSLG |
| | limab | | | YAMYWVRQAPGQGLEWIGVIRTYSGDVTYNQ | ERATINCKSSQSLLNS |
| | | | | KFKDRATMTVDKSISTAYMELSRLRSDDTAVY | GNQKNYLTWYQQKPGQ |
| | | | | YCAKSGTVRGFAYWGQGTLVTVSSASTKGPS | PPKLLIYWASTRESGV |
| | | | | VFPLAPSSKSTSGGTAALGCLVKDYFPEPVTV | PDRFSGSGSGTDFTLT |
| | | | | SWNSGALTSGVHTFPAVLQSSGLYSLSSVVTV | ISSLQAEDVAVYHCQN |
| | | | | PSSSLGTQTYICNVNHKPSNTKVDKRVEPKSC | DYSYPYTFGQGTKLEI |
| | | | | DKTHTCPPCPAPELLGGPSVFLFPPKPKDTLM | KRTVAAPSVFIFPPSD |
| | | | | ISRTPEVTCVVVDVSHEDPEVKFNWYVDGVE | EQLKSGTASVVCLLNN |
| | | | | VHNAKTKPREEQYNSTYRVVSVLTVLHQDWL | FYPREAKVQWKVDNAL |
| | | | | NGKEYKCKVSNKALPAPIEKTISKAKGQPREP | QSGNSQESVTEQDSKD |
| | | | | QVYTLPPSREEMTKNQVSLTCLVKGFYPSDIA | STYSLSSTLTLSKADY |
| | | | | VEWESNGQPENNYKTTPPVLDSDGSFFLYSK | EKHKVYACEVTHQGLS |
| | | | | LTVDKSRWQQGNVFSCSVMHEALHNHYTQK | SPVTKSFNRGEC |
| | | | | SLSLSPG | |
| GITR | TRX518 | 338 | 339 | QVTLRESGPALVKPTQTLTLTCTFSGFSLSTS | EIVMTQSPATLSVSPG |
| | | | | GMGVGWIRQPPGKALEWLAHIWWDDDKYYN | ERATLSCKASQNVGTN |
| | | | | PSLKSRLTISKDTSKNQVVLTMTNMDPVDTAT | VAWYQQKPGQAPRLLI |
| | | | | YYCARTRRYFPFAYWGQGTLVTVSSASTKGP | YSASYRYSGIPARFSG |
| | | | | SVFPLAPSSKSTSGGTAALGCLVKDYFPEPVT | SGSGTEFTLTISSLQS |
| | | | | VSWNSGALTSGVHTFPAVLQSSGLYSLSSVVT | EDFAVYYCQQYNTDPL |
| | | | | VPSSSLGTQTYICNVNHKPSNTKVDKKVEPKS | TFGGGTKVEIKRTVAA |
| | | | | CDKTHTCPPCPAPELLGGPSVFLFPPKPKDTL | PSVFIFPPSDEQLKSG |
| | | | | MISRTPEVTCVVVDVSHEDPEVKFNWYVDGV | TASVVCLLNNFYPREA |
| | | | | EVHNAKTKPREEQYNSTYRVVSVLTVLHQDW | KVQWKVDNALQSGNSQ |
| | | | | LNGKEYKCKVSNKALPAPIEKTISKAKGQPREP | ESVTEQDSKDSTYSLS |
| | | | | QVYTLPPSRDELTKNQVSLTCLVKGFYPSDIA | STLTLSKADYEKHKVY |
| | | | | VEWESNGQPENNYKTTPPVLDSDGSFFLYSK | ACEVTHQGLSSPVTKS |
| | | | | LTVDKSRWQQGNVFSCSVMHEALHNHYTQK | FNRGEC |
| | | | | SLSLSPGK | |
| FAP | Sibrotu- | 510 | 511 | QVQLVQSGAEVKKPGASVKVSCKTSRYTFTE | DIVMTQSPDSLAVSLG |
| | zumab-FAP | | | YTIHWVRQAPGQRLEWIGGINPNNGIPNYNQK | ERATINCKSSQSLLYS |
| | binding | | | FKGRVTITVDTSASTAYMELSSLRSEDTAVYY | RNQKNYLAWYQQKPGQ |
| | portion | | | CARRRIAYGYDEGHAMDYWGQGTLVTVSS | PPKLLIFWASTRESGV |
| | | | | | PDRFSGSGFGTDFTLT |
| | | | | | ISSLQAEDVAVYYCQQ |
| | | | | | YFSYPLTFGQGTKVEI |
| | | | | | K |
| FAP | Simluka- | 340 | 341 | EVQLLESGGGLVQPGGSLRLSCAASGFTFSS | EIVLTQSPGTLSLSPG |
| | fusp- | | | YAMSWVRQAPGKGLEWVSAIIGSGASTYYAD | ERATLSCRASQSVTSS |
| | heavy | | | SVKGRFTISRDNSKNTLYLQMNSLRAEDTAVY | YLAWYQQKPGQAPRLL |
| | chain 1: | | | YCAKGWFGGFNYWGQGTLVTVSSASTKGPS | INVGSRRATGIPDRFS |
| | anti-FAP | | | VFPLAPSSKSTSGGTAALGCLVKDYFPEPVTV | GSGSGTDFTLTISRLE |
| | fused to | | | SWNSGALTSGVHTFPAVLQSSGLYSLSSVVTV | PEDFAVYYCQQGIMLP |
| | IL-2 | | | PSSSLGTQTYICNVNHKPSNTKVDKKVEPKSC | PTFGQGTKVEIKRTVA |
| | | | | DKTHTCPPCPAPEAAGGPSVFLFPPKPKDTLM | APSVFIFPPSDEQLKS |
| | | | | ISRTPEVTCVVVDVSHEDPEVKFNWYVDGVE | GTASVVCLLNNFYPRE |
| | | | | VHNAKTKPREEQYNSTYRVVSVLTVLHQDWL | AKVQWKVDNALQSGNS |
| | | | | NGKEYKCKVSNKALGAPIEKTISKAKGQPREP | QESVTEQDSKDSTYSL |
| | | | | QVYTLPPCRDELTKNQVSLWCLVKGFYPSDIA | SSTLTLSKADYEKHKV |
| | | | | VEWESNGQPENNYKTTPPVLDSDGSFFLYSK | YACEVTHQGLSSPVTK |
| | | | | LTVDKSRWQQGNVFSCSVMHEALHNHYTQK | SFNRGEC |
| | | | | SLSLSPGGGGGSGGGGSGGGGSAPASSSTK | |
| | | | | KTQLQLEHLLLDLQMILNGINNYKNPKLTRMLT | |
| | | | | AKFAMPKKATELKHLQCLEEELKPLEEVLNGA | |
| | | | | QSKNFHLRPRDLISNINVIVLELKGSETTFMCE | |
| | | | | YADETATIVEFLNRWITFAQSIISTLT | |
| FAP | Simluka- | 342 | 343 | EVQLLESGGGLVQPGGSLRLSCAASGFTFSS | EIVLTQSPGTLSLSPG |
| | fusp- | | | YAMSWVRQAPGKGLEWVSAIIGSGASTYYAD | ERATLSCRASQSVTSS |
| | heavy | | | SVKGRFTISRDNSKNTLYLQMNSLRAEDTAVY | YLAWYQQKPGQAPRLL |
| | chain 2: | | | YCAKGWFGGFNYWGQGTLVTVSSASTKGPS | INVGSRRATGIPDRFS |
| | anti-FAP | | | VFPLAPSSKSTSGGTAALGCLVKDYFPEPVTV | GSGSGTDFTLTISRLE |
| | | | | SWNSGALTSGVHTFPAVLQSSGLYSLSSVVTV | PEDFAVYYCQQGIMLP |
| | | | | PSSSLGTQTYICNVNHKPSNTKVDKKVEPKSC | PTFGQGTKVEIKRTVA |
| | | | | DKTHTCPPCPAPEAAGGPSVFLFPPKPKDTLM | APSVFIFPPSDEQLKS |
| | | | | ISRTPEVTCVVVDVSHEDPEVKFNWYVDGVE | GTASVVCLLNNFYPRE |
| | | | | VHNAKTKPREEQYNSTYRVVSVLTVLHQDWL | AKVQWKVDNALQSGNS |
| | | | | NGKEYKCKVSNKALGAPIEKTISKAKGQPREP | QESVTEQDSKDSTYSL |
| | | | | QVCTLPPSRDELTKNQVSLSCAVKGFYPSDIA | SSTLTLSKADYEKHKV |
| | | | | VEWESNGQPENNYKTTPPVLDSDGSFFLVSK | YACEVTHQGLSSPVTK |
| | | | | LTVDKSRWQQGNVFSCSVMHEALHNHYTQK | SFNRGEC |
| | | | | SLSLSPGK | |
| EpCAM | tucotu- | 344 | 345 | EIQLVQSGAEVKKPGETVKISCKASGYTFTNY | EIVLTQSPATLSLSPG |
| | zumab- | | | GMNWVKQTPGKGLKWMGWINTYTGEPTYAD | ERVTLTCSASSSVSYM |
| | fusion | | | DFKGRFAFSLETSTSTAFLQINNLRSEDTATYF | LWYQQKPGSSPKPWIF |
| | of anti- | | | CVRFISKGDYWGQGTSVTVSSASTKGPSVFP | DTSNLASGFPARFSGS |
| | EpCAM | | | LAPSSKSTSGGTAALGCLVKDYFPEPVTVSW | GSGTSYSLIISSMEAE |
| | and IL-2 | | | NSGALTSGVHTFPAVLQSSGLYSLSSVVTVPS | DAATYYCHQRSGYPYT |
| | | | | SSLGTQTYICNVNHKPSNTKVDKRVEPKSCDK | FGGGTKLEIKRTVAAP |
| | | | | THTCPPCPAPELLGGPSVFLFPPKPKDTLMIS | SVFIFPPSDEQLKSGT |
| | | | | RTPEVTCVVVDVSHEDPEVKFNWYVDGVEVH | ASVVCLLNNFYPREAK |
| | | | | NAKTKPREEQYNSTYRVVSVLTVLHQDWLNG | VQWKVDNALQSGNSQE |
| | | | | KEYKCKVSNKALPAPIEKTISKAKGQPREPQV | SVTEQDSKDSTYSLSS |
| | | | | YTLPPSREEMTKNQVSLTCLVKGFYPSDIAVE | TLTLSKADYEKHKVYA |
| | | | | WESNGQPENNYKTTPPVLDSDGSFFLYSKLT | CEVTHQGLSSPVTKSF |
| | | | | VDKSRWQQGNVFSCSVMHEALHNHYTQKSL | NRGEC |
| | | | | SLSPGKAPTSSSTKKTQLQLEHLLLDLQMILNG | |
| | | | | INNYKNPKLTRMLTFKFYMPKKATELKHLQCL | |
| | | | | EEELKPLEEVLNLAQSKNFHLRPRDLISNINVI | |
| | | | | VLELKGSETTFMCEYADETATIVEFLNRWITFC | |
| | | | | QSIISTLT | |
| IL-7R | Lusverti- | 346 | 347 | QVQLVESGGGLVKPGGSLRLSCAVSGFTLSD | DIQMTQSPSSLSASVG |
| (CD127) | kimab | | | YYMAWIRQAPGKGLEWVSTISASGLRTYYPD | DRVTITCRTSEDIYQG |
| | | | | SVKGRFTISRDNAKNSLYLQMNSLRAEDTAVY | LAWYQQKPGKAPKLLL |
| | | | | YCARPLSAHYGFNYFDYWGQGTLVTVSSAST | YSANTLHIGVPSRFSG |
| | | | | KGPSVFPLAPCSRSTSESTAALGCLVKDYFPE | SGSGTDYTLTISSLQP |
| | | | | PVTVSWNSGALTSGVHTFPAVLQSSGLYSLSS | EDFATYYCQQYYDYPL |
| | | | | VVTVPSSSLGTKTYTCNVDHKPSNTKVDKRVE | AFGGGTKVEIKRTVAA |
| | | | | SKYGPPCPPCPAPEFLGGPSVFLFPPKPKDTL | PSVFIFPPSDEQLKSG |
| | | | | MISRTPEVTCVVVDVSQEDPEVQFNWYVDGV | TASVVCLLNNFYPREA |
| | | | | EVHNAKTKPREEQFNSTYRVVSVLTVLHQDW | KVQWKVDNALQSGNSQ |
| | | | | LNGKEYKCKVSNKGLPSSIEKTISKAKGQPRE | ESVTEQDSKDSTYSLS |
| | | | | PQVYTLPPSQEEMTKNQVSLTCLVKGFYPSDI | STLTLSKADYEKHKVY |
| | | | | AVEWESNGQPENNYKTTPPVLDSDGSFFLYS | ACEVTHQGLSSPVTKS |
| | | | | RLTVDKSRWQEGNVFSCSVMHEALHNHYTQ | FNRGEC |
| | | | | KSLSLSPGK | |
| IL-7R | bempiki- | 348 | 349 | EVQLVESGGGLVQPGRSLRLSCAASGFTFDD | AIQLTQSPSSLSASVG |
| | bart | | | HAMHWVRQAPGKGLEWVSGISWNSRGIGYA | DRVTITCRASQGISSA |
| | | | | DSVKGRFTIFRDNAKNSLYLQMNSLRAEDTAL | LAWYQQKPGKAPKLLI |
| | | | | YYCAKDEYSRGYYVLDVWGQGTTVTVSSAST | YDASSLESGVPSRFSG |
| | | | | KGPSVFPLAPSSKSTSGGTAALGCLVKDYFPE | SGSGTDFTLTISSLQP |
| | | | | PVTVSWNSGALTSGVHTFPAVLQSSGLYSLSS | EDFATYYCQQFNSYPL |
| | | | | VVTVPSSSLGTQTYICNVNHKPSNTKVDKRVE | WITFGQGTRLEIKRTV |
| | | | | PKSCDKTHTCPPCPAPEAEGAPSVFLFPPKPK | AAPSVFIFPPSDEQLK |
| | | | | DTLMISRTPEVTCVVVDVSHEDPEVKFNWYVD | SGTASVVCLLNNFYPR |
| | | | | GVEVHNAKTKPREEQYNSTYRVVSVLTVLHQ | EAKVQWKVDNALQSGN |
| | | | | DWLNGKEYKCKVSNKALPAPIEKTISKAKGQP | SQESVTEQDSKDSTYS |
| | | | | REPQVYTLPPSREEMTKNQVSLTCLVKGFYPS | LSSTLTLSKADYEKHK |
| | | | | DIAVEWESNGQPENNYKTTPPVLDSDGSFFLY | VYACEVTHQGLSSPVT |
| | | | | SKLTVDKSRWQQGNVFSCSVMHEALHNHYT | KSFNRGEC |
| | | | | QKSLSLSPG | |
| IL-7R | PF- | 350 | 351 | EVQLVESGGGLVKPGGSLRLSCAASGFTFDD | NFMLTQPHSVSESPGK |
| | 06342647 | | | SVMHWVRQAPGKGLEWVSLVGWDGFFTYYA | TVTISCTRSSGSIDSS |
| | | | | DSVKGRFTISRDNAKNSLYLQMNSLRAEDTAV | YVQWYQQRPGSSPTTV |
| | | | | YYCARQGDYMGNNWGQGTLVTVSSASTKGP | IYEDDQRPSGVPDRFS |
| | | | | SVFPLAPSSKSTSGGTAALGCLVKDYFPEPVT | GSIDSSSNSASLTISG |
| | | | | VSWNSGALTSGVHTFPAVLQSSGLYSLSSVVT | LKTEDEADYYCQSYDF |
| | | | | VPSSSLGTQTYICNVNHKPSNTKVDKKVAPEL | HHLVFGGGTKLTVLQP |
| | | | | LGGPSVFLFPPKPKDTLMISRTPEVTCVVVDV | KAAPSVTLFPPSSEEL |
| | | | | SHEDPEVKFNWYVDGVEVHNAKTKPREEQYN | QANKATLVCLISDFYP |
| | | | | STYRVVSVLTVLHQDWLNGKEYKCKVSNKAL | GAVTVAWKADSSPVKA |
| | | | | PAPIEKTISKAKGQPREPQVYTLPPSREEMTK | GVETTTPSKQSNNKYA |
| | | | | NQVSLTCLVKGFYPSDIAVEWESNGQPENNY | ASSYLSLTPEQWKSHR |
| | | | | KTTPPVLDSDGSFFLYSKLTVDKSRWQQGNV | SYSCQVTHEGSTVEKT |
| | | | | FSCSVMHEALHNHYTQKSLSLSPGK | VAPTECS |
| IL-7R | alomfi- | 352 | 353 | EVQLVESGGGVVRPGGSLRLSCVASGVTFDD | EIVLTQSPGTLSLSPG |
| | limab | | | YGMSWVRQAPGKGLEWVSGINWNGGDTDYS | ERATLSCRASQSVSRS |
| | | | | DSVKGRFTISRDNAKNSLYLQMNSLRAEDTAL | YLAWYQQKRGQAPRLL |
| | | | | YYCARDFYGSGSYYHVPFDYWGQGILVTVSS | IYGASSRATGIPDRFS |
| | | | | ASTKGPSVFPLAPSSKSTSGGTAALGCLVKDY | GDGSGTDFTLSISRLE |
| | | | | FPEPVTVSWNSGALTSGVHTFPAVLQSSGLY | PEDFAVYYCHQYDMSP |
| | | | | SLSSVVTVPSSSLGTQTYICNVNHKPSNTKVD | FTFGPGTKVDIKRTVA |
| | | | | KKVEPKSCDKTHTCPPCPAPELLGGPSVFLFP | APSVFIFPPSDEQLKS |
| | | | | PKPKDTLMISRTPEVTCVVVDVSHEDPEVKFN | GTASVVCLLNNFYPRE |
| | | | | WYVDGVEVHNAKTKPREEQYNSTYRVVSVLT | AKVQWKVDNALQSGNS |
| | | | | VLHQDWLNGKEYKCKVSNKALPAPIEKTISKA | QESVTEQDSKDSTYSL |
| | | | | KGQPREPQVYTLPPSRDELTKNQVSLTCLVK | SSTLTLSKADYEKHKV |
| | | | | GFYPSDIAVEWESNGQPENNYKTTPPVLDSD | YACEVTHQGLSSPVTK |
| | | | | GSFFLYSKLTVDKSRWQQGNVFSCSVMHEAL | SFNRGEC |
| | | | | HNHYTQKSLSLSPGK | |
| LAG-3 | BI754111 | 354 | 355 | QVTLVESGGGVVQPGRSLRLSCAFSGFSLST | DIQMTQSPSFLSASVG |
| | | | | SDMGVGWIRQAPGKGLEWVAHIWWDDVKRY | DRVSITCKASQDVSTA |
| | | | | NPALKSRFTISRDNSKNTLYLQMNSLRAEDTA | VAWYQQKPGKAPKLLI |
| | | | | VYFCARIEDYGVSYYFDYWGQGTTVTVSS | YSASYRYTGVPDRFSG |
| | | | | | SGSGTDFTLTISSLQP |
| | | | | | EDFATYYCQQHYSIPL |
| | | | | | TFGQGTKLEIK |
| LAG-3 | encelimab | 356 | 357 | EVQLVQSGAEVKKPGATVKISCKASGFSIKDD | DIVMTQTPLSLSVTPG |
| | | | | YIHWVQQAPGKGLEWMGWIDAMNDDSQYSS | QPASISCRSSQSLVHS |
| | | | | KFQGRVTITVDTSTNTAYMKLSSLRSEDTAVY | DSNTYLHWYLQKPGQS |
| | | | | YCTYAFGGYWGQGTTVTVSSASTKGPSVFPL | PQLLIYLVSNRFSGVP |
| | | | | APCSRSTSESTAALGCLVKDYFPEPVTVSWN | DRFSGSGSGTDFTLKI |
| | | | | SGALTSGVHTFPAVLQSSGLYSLSSVVTVPSS | SRVEAEDVGVYFCGQS |
| | | | | SLGTKTYTCNVDHKPSNTKVDKRVESKYGPP | THVPYAFGGGTKVEIK |
| | | | | CPPCPAPEFLGGPSVFLFPPKPKDTLMISRTP | RTVAAPSVFIFPPSDE |
| | | | | EVTCVVVDVSQEDPEVQFNWYVDGVEVHNA | QLKSGTASVVCLLNNF |
| | | | | KTKPREEQFNSTYRVVSVLTVLHQDWLNGKE | YPREAKVQWKVDNALQ |
| | | | | YKCKVSNKGLPSSIEKTISKAKGQPREPQVYTL | SGNSQESVTEQDSKDS |
| | | | | PPSQEEMTKNQVSLTCLVKGFYPSDIAVEWES | TYSLSSTLTLSKADYE |
| | | | | NGQPENNYKTTPPVLDSDGSFFLYSRLTVDKS | KHKVYACEVTHQGLSS |
| | | | | RWQEGNVFSCSVMHEALHNHYTQKSLSLSLG | PVTKSFNRGEC |
| | | | | K | |
| LAG-3 | faveze- | 358 | 359 | QMQLVQSGPEVKKPGTSVKVSCKASGYTFTD | DIVMTQTPLSLSVTPG |
| | limab | | | YNVDWVRQARGQRLEWIGDINPNDGGTIYAQ | QPASISCKASQSLDYE |
| | | | | KFQERVTITVDKSTSTAYMELSSLRSEDTAVY | GDSDMNWYLQKPGQPP |
| | | | | YCARNYRWFGAMDHWGQGTTVTVSSASTKG | QLLIYGASNLESGVPD |
| | | | | PSVFPLAPCSRSTSESTAALGCLVKDYFPEPV | RFSGSGSGTDFTLKIS |
| | | | | TVSWNSGALTSGVHTFPAVLQSSGLYSLSSVV | RVEAEDVGVYYCQQST |
| | | | | TVPSSSLGTKTYTCNVDHKPSNTKVDKRVESK | EDPRTFGGGTKVEIKR |
| | | | | YGPPCPPCPAPEFLGGPSVFLFPPKPKDTLMI | TVAAPSVFIFPPSDEQ |
| | | | | SRTPEVTCVVVDVSQEDPEVQFNWYVDGVEV | LKSGTASVVCLLNNFY |
| | | | | HNAKTKPREEQFNSTYRVVSVLTVLHQDWLN | PREAKVQWKVDNALQS |
| | | | | GKEYKCKVSNKGLPSSIEKTISKAKGQPREPQ | GNSQESVTEQDSKDST |
| | | | | VYTLPPSQEEMTKNQVSLTCLVKGFYPSDIAV | YSLSSTLTLSKADYEK |
| | | | | EWESNGQPENNYKTTPPVLDSDGSFFLYSRL | HKVYACEVTHQGLSSP |
| | | | | TVDKSRWQEGNVFSCSVMHEALHNHYTQKSL | VTKSFNRGEC |
| | | | | SLSLGK | |
| LAG-3 | fianlimab | 360 | 361 | QVQLVESGGGVVQPGRSLRLSCVASGFTFSS | EIVLTQSPATLSLSPG |
| | | | | YGMHWVRQAPGKGLEWVAIIWYDGSNKYYA | ERTTLSCRASQRISTY |
| | | | | DSVKGRFTISRDNSKNTQYLQMNSLRAEDTAV | LAWYQQKPGQAPRLLI |
| | | | | YYCASVATSGDFDYYGMDVWGQGTTVTVSS | YDASKRATGIPARFSG |
| | | | | ASTKGPSVFPLAPCSRSTSESTAALGCLVKDY | SGSGTGFTLTISSLEP |
| | | | | FPEPVTVSWNSGALTSGVHTFPAVLQSSGLY | EDFAVYYCQQRSNWPL |
| | | | | SLSSVVTVPSSSLGTKTYTCNVDHKPSNTKVD | TFGGGTKVEIKRTVAA |
| | | | | KRVESKYGPPCPPCPAPPVAGPSVFLFPPKPK | PSVFIFPPSDEQLKSG |
| | | | | DTLMISRTPEVTCVVVDVSQEDPEVQFNWYV | TASVVCLLNNFYPREA |
| | | | | DGVEVHNAKTKPREEQFNSTYRVVSVLTVLH | KVQWKVDNALQSGNSQ |
| | | | | QDWLNGKEYKCKVSNKGLPSSIEKTISKAKGQ | ESVTEQDSKDSTYSLS |
| | | | | PREPQVYTLPPSQEEMTKNQVSLTCLVKGFY | STLTLSKADYEKHKVY |
| | | | | PSDIAVEWESNGQPENNYKTTPPVLDSDGSF | ACEVTHQGLSSPVTKS |
| | | | | FLYSRLTVDKSRWQEGNVFSCSVMHEALHNH | FNRGEC |
| | | | | YTQKSLSLSLGK | |
| LAG-3 | FS118 | 362 | 363 | EVQLVESGGGLVQPGRSLRLSCAASGFTFDD | DIQMTQSPSSLSASVG |
| | | | | YAMHWVRQTPGKGLEWVSGISWKSNIIGYAD | DRVTITCRASQSISSY |
| | | | | SVKGRFTISRDNAKNSLYLQMNSLRAEDTALY | LNWYQQKPGKAPKPLI |
| | | | | YCARDITGSGSYGWFDPWGQGTLVTVSSAST | YVASSLQSGVPSSFSG |
| | | | | KGPSVFPLAPSSKSTSGGTAALGCLVKDYFPE | SGSGTDFTLTISSLQP |
| | | | | PVTVSWNSGALTSGVHTFPAVLQSSGLYSLSS | EDFATYYCQQSYSNPI |
| | | | | VVTVPSSSLGTQTYICNVNHKPSNTKVDKKVE | TFGQGTRLEIKRTVAA |
| | | | | PKSCDKTHTCPPCPAPEAAGGPSVFLFPPKPK | PSVFIFPPSDEQLKSG |
| | | | | DTLMISRTPEVTCVVVDVSHEDPEVKFNWYVD | TASVVCLLNNFYPREA |
| | | | | GVEVHNAKTKPREEQYNSTYRVVSVLTVLHQ | KVQWKVDNALQSGNSQ |
| | | | | DWLNGKEYKCKVSNKALPAPIEKTISKAKGQP | ESVTEQDSKDSTYSLS |
| | | | | REPQVYTLPPSWDEPWGEDVSLTCLVKGFYP | STLTLSKADYEKHKVY |
| | | | | SDIAVEWESNGQPENNYKTTPPVLDSDGSFFL | ACEVTHQGLSSPVTKS |
| | | | | YSKLTVPYDRWVWPDEFSCSVMHEALHNHYT | FNRGEC |
| | | | | QKSLSLSPG | |
| LAG-3 | GSK | 364 | 365 | QVQLQESGPGLVKPSETLSLTCTVSGFSLTAY | DIQMTQSPSSLSASVG |
| | 2831781 | | | GVNWIRQPPGKGLEWIGMIWDDGSTDYDSAL | DRVTITCKSSQSLLNP |
| | | | | KSRVTISVDTSKNQFSLKLSSVTAADTAVYYCA | SNQKNYLAWYQQKPGK |
| | | | | REGDVAFDYWGQGTLVTVSSASTKGPSVFPL | APKLLVYFASTRDSGV |
| | | | | APSSKSTSGGTAALGCLVKDYFPEPVTVSWN | PSRFSGSGSGTDFTLT |
| | | | | SGALTSGVHTFPAVLQSSGLYSLSSVVTVPSS | ISSLQPEDFATYYCLQ |
| | | | | SLGTQTYICNVNHKPSNTKVDKKVEPKSCDKT | HFGTPPTFGQGTKLEI |
| | | | | HTCPPCPAPELLGGPSVFLFPPKPKDTLMISR | KRTVAAPSVFIFPPSD |
| | | | | TPEVTCVVVDVSHEDPEVKFNWYVDGVEVHN | EQLKSGTASVVCLLNN |
| | | | | AKTKPREEQYNSTYRVVSVLTVLHQDWLNGK | FYPREAKVQWKVDNAL |
| | | | | EYKCKVSNKALPAPIEKTISKAKGQPREPQVYT | QSGNSQESVTEQDSKD |
| | | | | LPPSRDELTKNQVSLTCLVKGFYPSDIAVEWE | STYSLSSTLTLSKADY |
| | | | | SNGQPENNYKTTPPVLDSDGSFFLYSKLTVDK | EKHKVYACEVTHQGLS |
| | | | | SRWQQGNVFSCSVMHEALHNHYTQKSLSLSP | SPVTKSFNRGEC |
| | | | | GK | |
| LAG-3 | ierami- | 366 | 367 | QVQLVQSGAEVKKPGASVKVSCKASGFTLTN | DIQMTQSPSSLSASVG |
| | limab | | | YGMNWVRQARGQRLEWIGWINTDTGEPTYA | DRVTITCSSSQDISNY |
| | | | | DDFKGRFVFSLDTSVSTAYLQISSLKAEDTAVY | LNWYLQKPGQSPQLLI |
| | | | | YCARNPPYYYGTNNAEAMDYWGQGTTVTVS | YYTSTLHLGVPSRFSG |
| | | | | SASTKGPSVFPLAPCSRSTSESTAALGCLVKD | SGSGTEFTLTISSLQP |
| | | | | YFPEPVTVSWNSGALTSGVHTFPAVLQSSGL | DDFATYYCQQYYNLPW |
| | | | | YSLSSVVTVPSSSLGTKTYTCNVDHKPSNTKV | TFGQGTKVEIKRTVAA |
| | | | | DKRVESKYGPPCPPCPAPEFLGGPSVFLFPPK | PSVFIFPPSDEQLKSG |
| | | | | PKDTLMISRTPEVTCVVVDVSQEDPEVQFNW | TASVVCLLNNFYPREA |
| | | | | YVDGVEVHNAKTKPREEQFNSTYRVVSVLTVL | KVQWKVDNALQSGNSQ |
| | | | | HQDWLNGKEYKCKVSNKGLPSSIEKTISKAKG | ESVTEQDSKDSTYSLS |
| | | | | QPREPQVYTLPPSQEEMTKNQVSLTCLVKGF | STLTLSKADYEKHKVY |
| | | | | YPSDIAVEWESNGQPENNYKTTPPVLDSDGS | ACEVTHQGLSSPVTKS |
| | | | | FFLYSRLTVDKSRWQEGNVFSCSVMHEALHN | FNRGEC |
| | | | | HYTQKSLSLSLG | |
| LAG-3 | tuparst- | 368 | 369 | QVQMVQSGAEVKKPGASVKVSCKASGFNIKD | EIVLTQSPGTLSLSPG |
| | obart | | | TYIHWVRQAPGQGLEWMGEIDPANDNTKYDP | ERATLSCSVSSSISSS |
| | | | | KFQGRVTITADTSTSTVYMELSSLRSEDTAVY | NLHWYQQKPGQAPRLL |
| | | | | YCATYYYKYDVGGFDYWGQGTLVTVSSASTK | IYGTSNLASGIPDRFS |
| | | | | GPSVFPLAPSSKSTSGGTAALGCLVKDYFPEP | GSGSGTDFTLTISRLE |
| | | | | VTVSWNSGALTSGVHTFPAVLQSSGLYSLSSV | PEDFAVYYCQQWSSYP |
| | | | | VTVPSSSLGTQTYICNVNHKPSNTKVDKRVEP | FTFGQGTKVEIKRTVA |
| | | | | KSCDKTHTCPPCPAPELLGGPSVFLFPPKPKD | APSVFIFPPSDEQLKS |
| | | | | TLMISRTPEVTCVVVDVSHEDPEVKFNWYVD | GTASVVCLLNNFYPRE |
| | | | | GVEVHNAKTKPREEQYASTYRVVSVLTVLHQ | AKVQWKVDNALQSGNS |
| | | | | DWLNGKEYKCKVSNKALPAPIEKTISKAKGQP | QESVTEQDSKDSTYSL |
| | | | | REPQVYTLPPSREEMTKNQVSLTCLVKGFYPS | SSTLTLSKADYEKHKV |
| | | | | DIAVEWESNGQPENNYKTTPPVLDSDGSFFLY | YACEVTHQGLSSPVTK |
| | | | | SKLTVDKSRWQQGNVFSCSVMHEALHNHYT | SF NRGEC |
| | | | | QKSLSLSPG | |
| LAG-3 | MGD013- | 370 | 371 | QVQLVQSGAEVKKPGASVKVSCKASGYSFTS | DIQMTQSPSSLSASVG |
| | LAG-3 | | | YWMNWVRQAPGQGLEWIGVIHPSDSETWLD | DRVTITCRASQDVSSV |
| | binding | | | QKFKDRVTITVDKSTSTAYMELSSLRSEDTAV | VAWYQQKPGKAPKLLI |
| | fragment | | | YYCAREHYGTSPFAYWGQGTLVTVSS | YSASYRYTGVPSRFSG |
| | | | | | SGSGTDFTLTISSLQP |
| | | | | | EDFATYYCQQHYSTPW |
| | | | | | TFGGGTKLEIK |
| LAG-3 | miptena- | 372 | 373 | QVTLVESGGGVVQPGRSLRLSCAFSGFSLST | DIQMTQSPSFLSASVG |
| | limab | | | SDMGVGWIRQAPGKGLEWVAHIWWDDVKRY | DRVSITCKASQDVSTA |
| | | | | NPALKSRFTISRDNSKNTLYLQMNSLRAEDTA | VAWYQQKPGKAPKLLI |
| | | | | VYFCARIEDYGVSYYFDYWGQGTTVTVSSAS | YSASYRYTGVPDRFSG |
| | | | | TKGPSVFPLAPCSRSTSESTAALGCLVKDYFP | SGSGTDFTLTISSLQP |
| | | | | EPVTVSWNSGALTSGVHTFPAVLQSSGLYSLS | EDFATYYCQQHYSIPL |
| | | | | SVVTVPSSSLGTKTYTCNVDHKPSNTKVDKRV | TFGQGTKLEIKRTVAA |
| | | | | ESKYGPPCPPCPAPEFLGGPSVFLFPPKPKDT | PSVFIFPPSDEQLKSG |
| | | | | LMISRTPEVTCVVVDVSQEDPEVQFNWYVDG | TASVVCLLNNFYPREA |
| | | | | VEVHNAKTKPREEQFNSTYRVVSVLTVLHQD | KVQWKVDNALQSGNSQ |
| | | | | WLNGKEYKCKVSNKGLPSSIEKTISKAKGQPR | ESVTEQDSKDSTYSLS |
| | | | | EPQVYTLPPSQEEMTKNQVSLTCLVKGFYPS | STLTLSKADYEKHKVY |
| | | | | DIAVEWESNGQPENNYKTTPPVLDSDGSFFLY | ACEVTHQGLSSPVTKS |
| | | | | SRLTVDKSRWQEGNVFSCSVMHEALHNHYT | FNRGEC |
| | | | | QKSLSLSLG | |
| LAG-3 | MK-4280- | 374 | 375 | QMQLVQSGPEVKKPGTSVKVSCKASGYTFTD | DIVMTQTPLSLSVTPG |
| | LAG-3 | | | YNVDWVRQARGQRLEWIGDINPNDGGTIYAQ | QPASISCKASQSLDYE |
| | binding | | | KFQERVTITVDKSTSTAYMELSSLRSEDTAVY | GDSDMNWYLQKPGQPP |
| | fragment | | | YCARNYRWFGAMDHWGQGTTVTVSS | QLLIYGASNLESGVPD |
| | | | | | RFSGSGSGTDFTLKIS |
| | | | | | RVEAEDVGVYYCQQST |
| | | | | | EDPRTFGGGTKVEIK |
| LAG-3 | REGN3767- | 376 | 377 | QVQLVESGGGVVQPGRSLRLSCVASGFTFSS | EIVLTQSPATLSLSPG |
| | LAG-3 | | | YGMHWVRQAPGKGLEWVAIIWYDGSNKYYA | ERTTLSCRASQRISTY |
| | binding | | | DSVKGRFTISRDNSKNTQYLQMNSLRAEDTAV | LAWYQQKPGQAPRLLI |
| | fragment | | | YYCASVATSGDFDYYGMDVWGQGTTVTVSS | YDASKRATGIPARFSG |
| | | | | | SGSGTGFTLTISSLEP |
| | | | | | EDFAVYYCQQ |
| LAG-3 | relatl- | 378 | 379 | QVQLQQWGAGLLKPSETLSLTCAVYGGSFSD | EIVLTQSPATLSLSPG |
| | imab | | | YYWNWIRQPPGKGLEWIGEINHRGSTNSNPS | ERATLSCRASQSISSY |
| | | | | LKSRVTLSLDTSKNQFSLKLRSVTAADTAVYY | LAWYQQKPGQAPRLLI |
| | | | | CAFGYSDYEYNWFDPWGQGTLVTVSSASTK | YDASNRATGIPARFSG |
| | | | | GPSVFPLAPCSRSTSESTAALGCLVKDYFPEP | SGSGTDFTLTISSLEP |
| | | | | VTVSWNSGALTSGVHTFPAVLQSSGLYSLSSV | EDFAVYYCQQRSNWPL |
| | | | | VTVPSSSLGTKTYTCNVDHKPSNTKVDKRVES | TFGQGTNLEIKRTVAA |
| | | | | KYGPPCPPCPAPEFLGGPSVFLFPPKPKDTLM | PSVFIFPPSDEQLKSG |
| | | | | ISRTPEVTCVVVDVSQEDPEVQFNWYVDGVE | TASVVCLLNNFYPREA |
| | | | | VHNAKTKPREEQFNSTYRVVSVLTVLHQDWL | KVQWKVDNALQSGNSQ |
| | | | | NGKEYKCKVSNKGLPSSIEKTISKAKGQPREP | ESVTEQDSKDSTYSLS |
| | | | | QVYTLPPSQEEMTKNQVSLTCLVKGFYPSDIA | STLTLSKADYEKHKVY |
| | | | | VEWESNGQPENNYKTTPPVLDSDGSFFLYSR | ACEVTHQGLSSPVTKS |
| | | | | LTVDKSRWQEGNVFSCSVMHEALHNHYTQKS | FNRGEC |
| | | | | LSLSLGK | |
| LAG-3 | Tebote- | 380 | 381 | DIQMTQSPSSLSASVGDRVTITCRASQDVSSV | EIVLTQSPATLSLSPG |
| | limab- | | | VAWYQQKPGKAPKLLIYSASYRYTGVPSRFS | ERATLSCRASESVDNY |
| | bispe- | | | GSGSGTDFTLTISSLQPEDFATYYCQQHYSTP | GMSFMNWFQQKPGQPP |
| | cific | | | WTFGGGTKLEIKGGGSGGGGQVQLVQSGAE | KLLIHAASNQGSGVPS |
| | anti- | | | VKKPGASVKVSCKASGYSFTSYWMNWVRQA | RFSGSGSGTDFTLTIS |
| | PD1 | | | PGQGLEWIGVIHPSDSETWLDQKFKDRVTITV | SLEPEDFAVYFCQQSK |
| | and | | | DKSTSTAYMELSSLRSEDTAVYYCAREHYGTS | EVPYTFGGGTKVEIKG |
| | anti- | | | PFAYWGQGTLVTVSSGGCGGGEVAACEKEV | GGSGGGGQVQLVQSGA |
| | LAG-3 | | | AALEKEVAALEKEVAALEKESKYGPPCPPCPA | EVKKPGASVKVSCKAS |
| | antibody | | | PEFLGGPSVFLFPPKPKDTLYITREPEVTCVVV | GYTFTDYNMDWVRQAP |
| | | | | DVSQEDPEVQFNWYVDGVEVHNAKTKPREE | GQGLEWMGDINPDNGV |
| | | | | QFNSTYRVVSVLTVLHQDWLNGKEYKCKVSN | TIYNQKFEGRVTMTTD |
| | | | | KGLPSSIEKTISKAKGQPREPQVYTLPPSQEE | TSTSTAYMELRSLRSD |
| | | | | MTKNQVSLTCLVKGFYPSDIAVEWESNGQPE | DTAVYYCAREADYFYF |
| | | | | NNYKTTPPVLDSDGSFFLYSRLTVDKSRWQE | DYWGQGTTLTVSSGGG |
| | | | | | GGGKVAACKEKVAALK |
| | | | | GNVFSCSVMHEALHNHYTQKSLSLSLG | EKVAALKEKVAALKE |
| LAG-3 | TSR-033 | 382 | 383 | EVQLVQSGAEVKKPGATVKISCKASGFSIKDD | DIVMTQTPLSLSVTPG |
| | | | | YIHWVQQAPGKGLEWMGWIDAMNDDSQYSS | QPASISCRSSQSLVHS |
| | | | | KFQGRVTITVDTSTNTAYMKLSSLRSEDTAVY | DSNTYLHWYLQKPGQS |
| | | | | YCTYAFGGYWGQGTTVTVSS | PQLLIYLVSNRFSGVP |
| | | | | | DRFSGSGSGTDFTLKI |
| | | | | | SRVEAEDVGVYFCGQS |
| | | | | | THVPYAFGGGTKVEIK |
| OX40 | BMS- | 384 | 385 | EVQLVESGGGLVQPGGSLRLSCAASGFTFSS | DIQMTQSPSSLSASVG |
| | 986178 | | | YSMNWVRQAPGKGLEWVSYISSSSSTIDYAD | DRVTITCRASQGISSW |
| | | | | SVKGRFTISRDNAKNSLYLQMNSLRDEDTAVY | LAWYQQKPEKAPKSLI |
| | | | | YCARESGWYLFDYWGQGTLVTVSS | YAASSLQSGVPSRFSG |
| | | | | | SGSGTDFTLTISSLQP |
| | | | | | EDFATYYCQQYNSYPP |
| | | | | | TFGGGTKVEIK |
| OX40 | cudaro- | 386 | 387 | QVQLVESGGGVVQPGRSLRLSCAASGFTFSS | DIQMTQSPSSLSASVG |
| | limab | | | YGMHWVRQAPGKGLEWVAVISYDGSNKYYA | DRVTITCQASQDISNY |
| | | | | DSVKGRFTISRDNSKNTLYLQMNSLRAEDTAV | LNWYQQKPGKAPKLLI |
| | | | | YYCARGRPWYSETGTSAFDIWGQGTMVTVSS | YDASNLETGVPSRFSG |
| | | | | ASTKGPSVFPLAPSSKSTSGGTAALGCLVKDY | SGSGTDFTFTISSLQP |
| | | | | FPEPVTVSWNSGALTSGVHTFPAVLQSSGLY | EDIATYYCQQSDHYPT |
| | | | | SLSSVVTVPSSSLGTQTYICNVNHKPSNTKVD | FGGGTKVEIKRTVAAP |
| | | | | KKVEPKSCDKTHTCPPCPAPELLGGPSVFLFP | SVFIFPPSDEQLKSGT |
| | | | | PKPKDTLMISRTPEVTCVVVDVSHEDPEVKFN | ASVVCLLNNFYPREAK |
| | | | | WYVDGVEVHNAKTKPREEQYNSTYRVVSVLT | VQWKVDNALQSGNSQE |
| | | | | VLHQDWLNGKEYKCKVSNKALPAPIEKTISKA | SVTEQDSKDSTYSLSS |
| | | | | KGQPREPQVYTLPPSRDELTKNQVSLTCLVK | TLTLSKADYEKHKVYA |
| | | | | GFYPSDIAVEWESNGQPENNYKTTPPVLDSD | CEVTHQGLSSPVTKSF |
| | | | | GSFFLYSKLTVDKSRWQQGNVFSCSVMHEAL | NRGEC |
| | | | | HNHYTQKSLSLSPG | |
| OX40 | ivuxo- | 388 | 389 | EVQLVESGGGLVQPGGSLRLSCAASGFTFSS | DIQMTQSPSSLSASVG |
| | limab | | | YSMNWVRQAPGKGLEWVSYISSSSSTIDYAD | DRVTITCRASQGISSW |
| | | | | SVKGRFTISRDNAKNSLYLQMNSLRDEDTAVY | LAWYQQKPEKAPKSLI |
| | | | | YCARESGWYLFDYWGQGTLVTVSSASTKGPS | YAASSLQSGVPSRFSG |
| | | | | VFPLAPCSRSTSESTAALGCLVKDYFPEPVTV | SGSGTDFTLTISSLQP |
| | | | | SWNSGALTSGVHTFPAVLQSSGLYSLSSVVTV | EDFATYYCQQYNSYPP |
| | | | | PSSNFGTQTYTCNVDHKPSNTKVDKTVERKC | TFGGGTKVEIKRTVAA |
| | | | | CVECPPCPAPPVAGPSVFLFPPKPKDTLMISR | PSVFIFPPSDEQLKSG |
| | | | | TPEVTCVVVDVSHEDPEVQFNWYVDGVEVHN | TASVVCLLNNFYPREA |
| | | | | AKTKPREEQFNSTFRVVSVLTVVHQDWLNGK | KVQWKVDNALQSGNSQ |
| | | | | EYKCKVSNKGLPAPIEKTISKTKGQPREPQVY | ESVTEQDSKDSTYSLS |
| | | | | TLPPSREEMTKNQVSLTCLVKGFYPSDIAVEW | STLTLSKADYEKHKVY |
| | | | | ESNGQPENNYKTTPPMLDSDGSFFLYSKLTV | ACEVTHQGLSSPVTKS |
| | | | | DKSRWQQGNVFSCSVMHEALHNHYTQKSLS | FNRGEC |
| | | | | LSPGK | |
| OX40 | revdofi- | 390 | 391 | EVQLVESGGGLVQPGGSLRLSCAASGFTFSR | DIVMTQSPDSLAVSLG |
| | limab | | | YGMSWVRQAPGKGLELVATINSNGGRTYYPD | ERATINCKASQSVDYD |
| | | | | SVKGRFTISRDNAKNSLYLQMNSLRAEDTAVY | GDSYMHWYQQKPGQPP |
| | | | | YCAREGITTAYAMDYWGQGTTVTVSSASTKG | KLLIYAASILESGVPD |
| | | | | PSVFPLAPSSKSTSGGTAALGCLVKDYFPEPV | RFSGSGSGTDFTLTIS |
| | | | | TVSWNSGALTSGVHTFPAVLQSSGLYSLSSVV | SLQAEDVAVYYCQQSN |
| | | | | TVPSSSLGTQTYICNVNHKPSNTKVDKKVEPK | EDPRTFGGGTKVEIKR |
| | | | | SCDKTHTCPPCPAPELLGGPSVFLFPPKPKDT | TVAAPSVFIFPPSDEQ |
| | | | | LMISRTPEVTCVVVDVSHEDPEVKFNWYVDG | LKSGTASVVCLLNNFY |
| | | | | VEVHNAKTKPREEQYNSTYRVVSVLTVLHQD | PREAKVQWKVDNALQS |
| | | | | WLNGKEYKCKVSNKALPAPIEKTISKAKGQPR | GNSQESVTEQDSKDST |
| | | | | EPQVYTLPPSREEMTKNQVSLTCLVKGFYPSD | YSLSSTLTLSKADYEK |
| | | | | IAVEWESNGQPENNYKTTPPVLDSDGSFFLYS | HKVYACEVTHQGLSSP |
| | | | | KLTVDKSRWQQGNVFSCSVMHEALHNHYTQ | VTKSFNRGEC |
| | | | | KSLSLSPGK | |
| OX40 | rocatin- | 392 | 393 | QITLKESGPTLVKPKQTLTLTCTFSGFSLSTSG | EIVLTQSPGTLSLSPG |
| | limab | | | MGVGWIRQPPGKALEWLAVIYWDDHQLYSPS | ERATLSCRASQSVSSS |
| | | | | LKSRLTITKDTSKNQVVLTMTNMDPVDTATYY | YLAWYQQKPGQAPRLL |
| | | | | CAHRRGAFQHWGQGTLVTVSSASTKGPSVFP | IYGASSRATGIPDRFS |
| | | | | LAPSSKSTSGGTAALGCLVKDYFPEPVTVSW | GSGSGTDFTLTISRLE |
| | | | | NSGALTSGVHTFPAVLQSSGLYSLSSVVTVPS | PEDFAVYYCQQYDSSL |
| | | | | SSLGTQTYICNVNHKPSNTKVDKKVEPKSCDK | TFGGGTKVEIKRTVAA |
| | | | | THTCPPCPAPELLGGPSVFLFPPKPKDTLMIS | PSVFIFPPSDEQLKSG |
| | | | | RTPEVTCVVVDVSHEDPEVKFNWYVDGVEVH | TASVVCLLNNFYPREA |
| | | | | NAKTKPREEQYNSTYRVVSVLTVLHQDWLNG | KVQWKVDNALQSGNSQ |
| | | | | KEYKCKVSNKALPAPIEKTISKAKGQPREPQV | ESVTEQDSKDSTYSLS |
| | | | | YTLPPSRDELTKNQVSLTCLVKGFYPSDIAVE | STLTLSKADYEKHKVY |
| | | | | WESNGQPENNYKTTPPVLDSDGSFFLYSKLT | ACEVTHQGLSSPVTKS |
| | | | | VDKSRWQQGNVFSCSVMHEALHNHYTQKSL | FNRGEC |
| | | | | SLSPGK | |
| OX40 | tavolimab | 394 | 395 | QVQLQESGPGLVKPSQTLSLTCAVYGGSFSS | DIQMTQSPSSLSASVG |
| | | | | GYWNWIRKHPGKGLEYIGYISYNGITYHNPSL | DRVTITCRASQDISNY |
| | | | | KSRITINRDTSKNQYSLQLNSVTPEDTAVYYCA | LNWYQQKPGKAPKLLI |
| | | | | RYKYDYDGGHAMDYWGQGTLVTVSSASTKG | YYTSKLHSGVPSRFSG |
| | | | | PSVFPLAPSSKSTSGGTAALGCLVKDYFPEPV | SGSGTDYTLTISSLQP |
| | | | | TVSWNSGALTSGVHTFPAVLQSSGLYSLSSVV | EDFATYYCQQGSALPW |
| | | | | TVPSSSLGTQTYICNVNHKPSNTKVDKRVEPK | TFGQGTKVEIKRTVAA |
| | | | | SCDKTHTCPPCPAPELLGGPSVFLFPPKPKDT | PSVFIFPPSDEQLKSG |
| | | | | LMISRTPEVTCVVVDVSHEDPEVKFNWYVDG | TASVVCLLNNFYPREA |
| | | | | VEVHNAKTKPREEQYNSTYRVVSVLTVLHQD | KVQWKVDNALQSGNSQ |
| | | | | WLNGKEYKCKVSNKALPAPIEKTISKAKGQPR | ESVTEQDSKDSTYSLS |
| | | | | EPQVYTLPPSREEMTKNQVSLTCLVKGFYPSD | STLTLSKADYEKHKVY |
| | | | | IAVEWESNGQPENNYKTTPPVLDSDGSFFLYS | ACEVTHQGLSSPVTKS |
| | | | | KLTVDKSRWQQGNVFSCSVMHEALHNHYTQ | FNRGEC |
| | | | | KSLSLSPGK | |
| OX40 | telazor- | 396 | 397 | QVTLKESGPALVKPTQTLTLTCSFSGFSLSTS | EIVLTQSPATLSLSPG |
| | limab | | | GMGVGWIRQPPGKALEWIAHIWWDDDKYYNT | ERATLSCRASSSVSYM |
| | | | | ALKTRLTISKDTSKNQVVLTMTNMDPVDTATY | HWYQQKPGQAPRPWIY |
| | | | | YCARIDWDGFAYWGQGTLVTVSSASTKGPSV | ATSNRATGIPARFSGS |
| | | | | FPLAPSSKSTSGGTAALGCLVKDYFPEPVTVS | GSGTDYTLTISSLEPE |
| | | | | WNSGALTSGVHTFPAVLQSSGLYSLSSVVTVP | DFAVYYCQQWSSNPWT |
| | | | | SSSLGTQTYICNVNHKPSNTKVDKKVEPKSCD | FGQGTKVEIKRTVAAP |
| | | | | KTHTCPPCPAPELLGGPSVFLFPPKPKDTLMIS | SVFIFPPSDEQLKSGT |
| | | | | RTPEVTCVVVDVSHEDPEVKFNWYVDGVEVH | ASVVCLLNNFYPREAK |
| | | | | NAKTKPREEQYNSTYRVVSVLTVLHQDWLNG | VQWKVDNALQSGNSQE |
| | | | | KEYKCKVSNKALPAPIEKTISKAKGQPREPQV | SVTEQDSKDSTYSLSS |
| | | | | YTLPPSRDELTKNQVSLTCLVKGFYPSDIAVE | TLTLSKADYEKHKVYA |
| | | | | WESNGQPENNYKTTPPVLDSDGSFFLYSKLT | CEVTHQGLSSPVTKSF |
| | | | | VDKSRWQQGNVFSCSVMHEALHNHYTQKSL | NRGEC |
| | | | | SLSPGK | |
| OX40 | Vonlero- | 398 | 399 | EVQLVQSGAEVKKPGASVKVSCKASGYTFTD | DIQMTQSPSSLSASVG |
| | lizumab | | | SYMSWVRQAPGQGLEWIGDMYPDNGDSSYN | DRVTITCRASQDISNY |
| | (MOXR0916) | | | QKFRERVTITRDTSTSTAYLELSSLRSEDTAVY | LNWYQQKPGKAPKLLI |
| | | | | YCVLAPRWYFSVWGQGTLVTVSSASTKGPSV | YYTSRLRSGVPSRFSG |
| | | | | FPLAPSSKSTSGGTAALGCLVKDYFPEPVTVS | SGSGTDFTLTISSLQP |
| | | | | WNSGALTSGVHTFPAVLQSSGLYSLSSVVTVP | EDFATYYCQQGHTLPP |
| | | | | SSSLGTQTYICNVNHKPSNTKVDKKVEPKSCD | TFGQGTKVEIKRTVAA |
| | | | | KTHTCPPCPAPELLGGPSVFLFPPKPKDTLMIS | PSVFIFPPSDEQLKSG |
| | | | | RTPEVTCVVVDVSHEDPEVKFNWYVDGVEVH | TASVVCLLNNFYPREA |
| | | | | NAKTKPREEQYNSTYRVVSVLTVLHQDWLNG | KVQWKVDNALQSGNSQ |
| | | | | KEYKCKVSNKALPAPIEKTISKAKGQPREPQV | ESVTEQDSKDSTYSLS |
| | | | | YTLPPSREEMTKNQVSLTCLVKGFYPSDIAVE | STLTLSKADYEKHKVY |
| | | | | WESNGQPENNYKTTPPVLDSDGSFFLYSKLT | ACEVTHQGLSSPVTKS |
| | | | | VDKSRWQQGNVFSCSVMHEALHNHYTQKSL | FNRGEC |
| | | | | SLSPGK | |
| PD-1 | acrixo- | 400 | 401 | QVQLVESGGGVVQPGRSLRLSCAASGFTFLR | DIVMTQTPLSLPVTPG |
| | limab | | | YAMHWVRQAPGKGLEWVAVISYDGRYKYYA | EAASISCRSSQSLLDS |
| | | | | DSVKGRFTISRDNSKNTLYLQMNSLRAEDTAV | EDGNTYLDWYLQKPGQ |
| | | | | YYCTTTTFDSWGQGTLVTVSSASTKGPSVFPL | SPQLLIYTLSHRASGV |
| | | | | APCSRSTSESTAALGCLVKDYFPEPVTVSWN | PDRFSGSGSGTDFTLE |
| | | | | SGALTSGVHTFPAVLQSSGLYSLSSVVTVPSS | ISRVEAEDVGVYYCMQ |
| | | | | SLGTKTYTCNVDHKPSNTKVDKRVESKYGPP | RRDFPFTFGQGTKVDI |
| | | | | CPPCPAPEFLGGPSVFLFPPKPKDTLMISRTP | KRTVAAPSVFIFPPSD |
| | | | | EVTCVVVDVSQEDPEVQFNWYVDGVEVHNA | EQLKSGTASVVCLLNN |
| | | | | KTKPREEQFNSTYRVVSVLTVLHQDWLNGKE | FYPREAKVQWKVDNAL |
| | | | | YKCKVSNKGLPSSIEKTISKAKGQPREPQVYTL | QSGNSQESVTEQDSKD |
| | | | | PPSQEEMTKNQVSLTCLVKGFYPSDIAVEWES | STYSLSSTLTLSKADY |
| | | | | NGQPENNYKTTPPVLDSDGSFFLYSRLTVDKS | EKHKVYACEVTHQGLS |
| | | | | RWQEGNVFSCSVMHEALHNHYTQKSLSLSLG | SPVTKSFNRGEC |
| | | | | K | |
| PD-1 | balsti- | 402 | 403 | QVQLVESGGGVVQPGRSLRLSCAASGFTFSS | EIVMTQSPATLSVSPG |
| | limab | | | YGMHWVRQAPGKGLEWVAVIWYDGSNKYYA | ERATLSCRASQSVSSN |
| | | | | DSVKGRFTISRDNSKNTLYLQMNSLRAEDTAV | LAWYQQKPGQAPRLLI |
| | | | | YYCASNGDHWGQGTLVTVSSASTKGPSVFPL | YGASTRATGIPARFSG |
| | | | | APCSRSTSESTAALGCLVKDYFPEPVTVSWN | SGSGTEFTLTISSLQS |
| | | | | SGALTSGVHTFPAVLQSSGLYSLSSVVTVPSS | EDFAVYYCQQYNNWPR |
| | | | | SLGTKTYTCNVDHKPSNTKVDKRVESKYGPP | TFGQGTKVEIKRTVAA |
| | | | | CPPCPAPEFLGGPSVFLFPPKPKDTLMISRTP | PSVFIFPPSDEQLKSG |
| | | | | EVTCVVVDVSQEDPEVQFNWYVDGVEVHNA | TASVVCLLNNFYPREA |
| | | | | KTKPREEQFNSTYRVVSVLTVLHQDWLNGKE | KVQWKVDNALQSGNSQ |
| | | | | YKCKVSNKGLPSSIEKTISKAKGQPREPQVYTL | ESVTEQDSKDSTYSLS |
| | | | | PPSQEEMTKNQVSLTCLVKGFYPSDIAVEWES | STLTLSKADYEKHKVY |
| | | | | NGQPENNYKTTPPVLDSDGSFFLYSRLTVDKS | ACEVTHQGLSSPVTKS |
| | | | | RWQEGNVFSCSVMHEALHNHYTQKSLSLSLG | FNRGEC |
| | | | | K | |
| PD-1 | budiga- | 404 | 405 | EIQLVQSGAEVKKPGSSVKVSCKASGYTFTHY | DVVMTQSPLSLPVTPG |
| | limab | | | GMNWVRQAPGQGLEWVGWVNTYTGEPTYA | EPASISCRSSQSIVHS |
| | | | | DDFKGRLTFTLDTSTSTAYMELSSLRSEDTAV | HGDTYLEWYLQKPGQS |
| | | | | YYCTREGEGLGFGDWGQGTTVTVSSASTKG | PQLLIYKVSNRFSGVP |
| | | | | PSVFPLAPSSKSTSGGTAALGCLVKDYFPEPV | DRFSGSGSGTDFTLKI |
| | | | | TVSWNSGALTSGVHTFPAVLQSSGLYSLSSVV | SRVEAEDVGVYYCFQG |
| | | | | TVPSSSLGTQTYICNVNHKPSNTKVDKKVEPK | SHIPVTFGQGTKLEIK |
| | | | | SCDKTHTCPPCPAPEAAGGPSVFLFPPKPKDT | RTVAAPSVFIFPPSDE |
| | | | | LMISRTPEVTCVVVDVSHEDPEVKFNWYVDG | QLKSGTASVVCLLNNF |
| | | | | VEVHNAKTKPREEQYNSTYRVVSVLTVLHQD | YPREAKVQWKVDNALQ |
| | | | | WLNGKEYKCKVSNKALPAPIEKTISKAKGQPR | SGNSQESVTEQDSKDS |
| | | | | EPQVYTLPPSREEMTKNQVSLTCLVKGFYPSD | TYSLSSTLTLSKADYE |
| | | | | IAVEWESNGQPENNYKTTPPVLDSDGSFFLYS | KHKVYACEVTHQGLSS |
| | | | | KLTVDKSRWQQGNVFSCSVMHEALHNHYTQ | PVTKSFNRGEC |
| | | | | KSLSLSPGK | |
| PD-1 | Cadoni- | 406 | 407 | EVQLVESGGGLVQPGGSLRLSCAASGFAFSS | DIQMTQSPSSMSASVG |
| | limab- | | | YDMSWVRQAPGKGLDWVATISGGGRYTYYP | DRVTFTCRASQDINTY |
| | bispecific | | | DSVKGRFTISRDNSKNNLYLQMNSLRAEDTAL | LSWFQQKPGKSPKTLI |
| | anti-PD1 | | | YYCANRYGEAWFAYWGQGTLVTVSSASTKG | YRANRLVSGVPSRFSG |
| | and | | | PSVFPLAPSSKSTSGGTAALGCLVKDYFPEPV | SGSGQDYTLTISSLQP |
| | anti-CTLA- | | | TVSWNSGALTSGVHTFPAVLQSSGLYSLSSVV | EDMATYYCLQYDEFPL |
| | 4 | | | TVPSSSLGTQTYICNVNHKPSNTKVDKKVEPK | TFGAGTKLELKRTVAA |
| | antibody | | | SCDKTHTCPPCPAPEAAGAPSVFLFPPKPKDT | PSVFIFPPSDEQLKSG |
| | | | | LMISRTPEVTCVVVDVSHEDPEVKFNWYVDG | TASVVCLLNNFYPREA |
| | | | | VEVHNAKTKPREEQYNSTYRVVSVLTVLHQD | KVQWKVDNALQSGNSQ |
| | | | | WLNGKEYKCKVSNKALPAPIEKTISKAKGQPR | ESVTEQDSKDSTYSLS |
| | | | | EPQVYTLPPSRDELTKNQVSLTCLVKGFYPSD | STLTLSKADYEKHKVY |
| | | | | IAVEWESNGQPENNYKTTPPVLDSDGSFFLYS | ACEVTHQGLSSPVTKS |
| | | | | KLTVDKSRWQQGNVFSCSVMHEALHNHYTQ | FNRGEC |
| | | | | KSLSLSPGKGGGGSGGGGSGGGGSGGGGS | |
| | | | | QVQLVESGAEVKKPGASVKVSCKASGYSFTG | |
| | | | | YTMNWVRQAPGQCLEWIGLINPYNNITNYAQK | |
| | | | | FQGRVTFTVDTSISTAYMELSRLRSDDTGVYF | |
| | | | | CARLDYRSYWGQGTLVTVSAGGGGSGGGGS | |
| | | | | GGGGSGGGGSQAVVTQEPSLTVSPGGTVTLT | |
| | | | | CGSSTGAVTTSNFPNWVQQKPGQAPRSLIGG | |
| | | | | TNNKASWTPARFSGSLLGGKAALTISGAQPED | |
| | | | | EAEYYCALWYSNHWVFGCGTKLTVLR | |
| PD-1 | camrel- | 408 | 409 | EVQLVESGGGLVQPGGSLRLSCAASGFTFSS | DIQMTQSPSSLSASVG |
| | izumab | | | YMMSWVRQAPGKGLDWVATISGGGANTYYP | DRVTITCLASQTIGTW |
| | | | | DSVKGRFTISRDNSKNNLYLQMNSLRAEDTAL | LTWYQQKPGKAPKLLI |
| | | | | YYCARQLYYFDYWGQGTTVTVSSASTKGPSV | YTATSLADGVPSRFSG |
| | | | | FPLAPCSRSTSESTAALGCLVKDYFPEPVTVS | SGSGTDFTLTISSLQP |
| | | | | WNSGALTSGVHTFPAVLQSSGLYSLSSVVTVP | EDFATYYCQQVYSIPW |
| | | | | SSSLGTKTYTCNVDHKPSNTKVDKRVESKYG | TFGGGTKVEIKRTVAA |
| | | | | PPCPPCPAPEFLGGPSVFLFPPKPKDTLMISR | PSVFIFPPSDEQLKSG |
| | | | | TPEVTCVVVDVSQEDPEVQFNWYVDGVEVHN | TASVVCLLNNFYPREA |
| | | | | AKTKPREEQFNSTYRVVSVLTVLHQDWLNGK | KVQWKVDNALQSGNSQ |
| | | | | EYKCKVSNKGLPSSIEKTISKAKGQPREPQVY | ESVTEQDSKDSTYSLS |
| | | | | TLPPSQEEMTKNQVSLTCLVKGFYPSDIAVEW | STLTLSKADYEKHKVY |
| | | | | ESNGQPENNYKTTPPVLDSDGSFFLYSRLTVD | ACEVTHQGLSSPVTKS |
| | | | | KSRWQEGNVFSCSVMHEALHNHYTQKSLSLS | FNRGEC |
| | | | | LGK | |
| PD-1 | cemiplimab | 410 | 411 | EVQLLESGGVLVQPGGSLRLSCAASGFTFSNF | DIQMTQSPSSLSASVG |
| | | | | GMTWVRQAPGKGLEWVSGISGGGRDTYFAD | DSITITCRASLSINTF |
| | | | | SVKGRFTISRDNSKNTLYLQMNSLKGEDTAVY | LNWYQQKPGKAPNLLI |
| | | | | YCVKWGNIFDYWGQGTLVTVSSASTKGPSV | YAASSLHGGVPSRFSG |
| | | | | FPLAPCSRSTSESTAALGCLVKDYFPEPVTVS | SGSGTDFTLTIRTLQP |
| | | | | WNSGALTSGVHTFPAVLQSSGLYSLSSVVTVP | EDFATYYCQQSSNTPF |
| | | | | SSSLGTKTYTCNVDHKPSNTKVDKRVESKYG | TFGPGTVVDFRRTVAA |
| | | | | PPCPPCPAPEFLGGPSVFLFPPKPKDTLMISR | PSVFIFPPSDEQLKSG |
| | | | | TPEVTCVVVDVSQEDPEVQFNWYVDGVEVHN | TASVVCLLNNFYPREA |
| | | | | AKTKPREEQFNSTYRVVSVLTVLHQDWLNGK | KVQWKVDNALQSGNSQ |
| | | | | EYKCKVSNKGLPSSIEKTISKAKGQPREPQVY | ESVTEQDSKDSTYSLS |
| | | | | TLPPSQEEMTKNQVSLTCLVKGFYPSDIAVEW | STLTLSKADYEKHKVY |
| | | | | ESNGQPENNYKTTPPVLDSDGSFFLYSRLTVD | ACEVTHQGLSSPVTKS |
| | | | | KSRWQEGNVFSCSVMHEALHNHYTQKSLSLS | FNRGEC |
| | | | | LGK | |
| PD-1 | cetrelimab | 412 | 413 | QVQLVQSGAEVKKPGSSVKVSCKASGGTFSS | EIVLTQSPATLSLSPG |
| | | | | YAISWVRQAPGKGLEWMGGIIPIFDTANYAQK | ERATLSCRASQSVRSY |
| | | | | FQGRVTITADESTSTAYMELSSLRSEDTAVYY | LAWYQQKPGQAPRLLI |
| | | | | CARPGLAAAYDTGSLDYWGQGTLVTVSSAST | YDASNRATGIPARFSG |
| | | | | KGPSVFPLAPCSRSTSESTAALGCLVKDYFPE | SGSGTDFTLTISSLEP |
| | | | | PVTVSWNSGALTSGVHTFPAVLQSSGLYSLSS | EDFAVYYCQQRNYWPL |
| | | | | VVTVPSSSLGTKTYTCNVDHKPSNTKVDKRVE | TFGQGTKVEIKRTVAA |
| | | | | SKYGPPCPPCPAPEFLGGPSVFLFPPKPKDTL | PSVFIFPPSDEQLKSG |
| | | | | MISRTPEVTCVVVDVSQEDPEVQFNWYVDGV | TASVVCLLNNFYPREA |
| | | | | EVHNAKTKPREEQFNSTYRVVSVLTVLHQDW | KVQWKVDNALQSGNSQ |
| | | | | LNGKEYKCKVSNKGLPSSIEKTISKAKGQPRE | ESVTEQDSKDSTYSLS |
| | | | | PQVYTLPPSQEEMTKNQVSLTCLVKGFYPSDI | STLTLSKADYEKHKVY |
| | | | | AVEWESNGQPENNYKTTPPVLDSDGSFFLYS | ACEVTHQGLSSPVTKS |
| | | | | RLTVDKSRWQEGNVFSCSVMHEALHNHYTQ | FNRGEC |
| | | | | KSLSLSLGK | |
| PD-1 | dostarl- | 414 | 415 | EVQLLESGGGLVQPGGSLRLSCAASGFTFSS | DIQLTQSPSFLSAYVG |
| | imab | | | YDMSWVRQAPGKGLEWVGSTISGGGSYTYYQ | DRVTITCKASQDVGTA |
| | | | | DSVKGRFTISRDNSKNTLYLQMNSLRAEDTAV | VAWYQQKPGKAPKLLI |
| | | | | YYCASPYYAMDYWGQGTTVTVSSASTKGPSV | YWASTLHTGVPSRFSG |
| | | | | FPLAPCSRSTSESTAALGCLVKDYFPEPVTVS | SGSGTEFTLTISSLQP |
| | | | | WNSGALTSGVHTFPAVLQSSGLYSLSSVVTVP | EDFATYYCQHYSSYPW |
| | | | | SSSLGTKTYTCNVDHKPSNTKVDKRVESKYG | TFGQGTKLEIKRTVAA |
| | | | | PPCPPCPAPEFLGGPSVFLFPPKPKDTLMISR | PSVFIFPPSDEQLKSG |
| | | | | TPEVTCVVVDVSQEDPEVQFNWYVDGVEVHN | TASVVCLLNNFYPREA |
| | | | | AKTKPREEQFNSTYRVVSVLTVLHQDWLNGK | KVQWKVDNALQSGNSQ |
| | | | | EYKCKVSNKGLPSSIEKTISKAKGQPREPQVY | ESVTEQDSKDSTYSLS |
| | | | | TLPPSQEEMTKNQVSLTCLVKGFYPSDIAVEW | STLTLSKADYEKHKVY |
| | | | | ESNGQPENNYKTTPPVLDSDGSFFLYSRLTVD | ACEVTHQGLSSPVTKS |
| | | | | KSRWQEGNVFSCSVMHEALHNHYTQKSLSLS | FNRGEC |
| | | | | LGK | |
| PD-1 | ezabenl- | 416 | 417 | EVMLVESGGGLVQPGGSLRLSCTASGFTFSK | EIVLTQSPATLSLSPG |
| | imab | | | SAMSWVRQAPGKGLEWVAYISGGGGDTYYS | ERATMSCRASENIDVS |
| | | | | SSVKGRFTISRDNAKNSLYLQMNSLRAEDTAV | GISFMNWYQQKPGQAP |
| | | | | YYCARHSNVNYYAMDYWGQGTLVTVSSASTK | KLLIYVASNQGSGIPA |
| | | | | GPSVFPLAPCSRSTSESTAALGCLVKDYFPEP | RFSGSGSGTDFTLTIS |
| | | | | VTVSWNSGALTSGVHTFPAVLQSSGLYSLSSV | RLEPEDFAVYYCQQSK |
| | | | | VTVPSSSLGTKTYTCNVDHKPSNTKVDKRVES | EVPWTFGQGTKLEIKR |
| | | | | KYGPPCPPCPAPEFLGGPSVFLFPPKPKDTLM | TVAAPSVFIFPPSDEQ |
| | | | | ISRTPEVTCVVVDVSQEDPEVQFNWYVDGVE | LKSGTASVVCLLNNFY |
| | | | | VHNAKTKPREEQFNSTYRVVSVLTVLHQDWL | PREAKVQWKVDNALQS |
| | | | | NGKEYKCKVSNKGLPSSIEKTISKAKGQPREP | GNSQESVTEQDSKDST |
| | | | | QVYTLPPSQEEMTKNQVSLTCLVKGFYPSDIA | YSLSSTLTLSKADYEK |
| | | | | VEWESNGQPENNYKTTPPVLDSDGSFFLYSR | HKVYACEVTHQGLSSP |
| | | | | LTVDKSRWQEGNVFSCSVMHEALHNHYTQKS | VTKSFNRGEC |
| | | | | LSLSLG | |
| PD-1 | fidasim- | 418 | 419 | QVQLVQSGAEVKKPGSSVKVSCKASGGTFSS | DIQMTQSPSSVSASVG |
| | tamab | | | TAISWVRQAPGQGLEWMGGIWPSFGTASYA | DRVTITCRASQGISSW |
| | | | | QKFQGRVTITADESTSTAYMELSSLRSEDTAV | LAWYQQKPGKAPKLLI |
| | | | | YYCARAEYSSTGIFDYWGQGTLVTVSSASTK | SAASSLQSGVPSRFSG |
| | | | | GPSVFPLAPSSKSTSGGTAALGCLVKDYFPEP | SGSGTDFTLTISSLQP |
| | | | | VTVSWNSGALTSGVHTFPAVLQSSGLYSLSSV | EDFATYYCQQANHLPF |
| | | | | VTVPSSSLGTQTYICNVNHKPSNTKVDKKVEP | TFGGGTKVEIKRTVAA |
| | | | | KSCDKTHTCPPCPAPELLGGPSVFLFPPKPKD | PSVFIFPPSDEQLKSG |
| | | | | TLMISRTPEVTCVVVDVSHEDPEVKFNWYVD | TASVVCLLNNFYPREA |
| | | | | GVEVHNAKTKPREEQYNSTYRVVSVLTVLHQ | KVQWKVDNALQSGNSQ |
| | | | | DWLNGKEYKCKVSNKALPAPIEKTISKAKGQP | ESVTEQDSKDSTYSLS |
| | | | | REPQVYTLPPSRDELTKNQVSLLCLVKGFYPS | STLTLSKADYEKHKVY |
| | | | | DIAVEWESNGQPENNYKTTPPVLRSDGSFFLY | ACEVTHQGLSSPVTKS |
| | | | | SKLTVDKSRWQQGNVFSCSVMHEALHNHYT | FNRGEC |
| | | | | QKSLSLSPGK | |
| PD-1 | finoton- | 420 | 421 | EVQLVESGGGLVKPGGSLRLSCAASGFTFSS | EIVLTQSPATLSLSPG |
| | limab | | | YGMSWVRQAPGKGLEWVATISGGGRDTYYS | ERATLSCRASESVDSY |
| | | | | DSVKGRFTISRDNAKNNLYLQMNSLRAEDTAV | GNSFMHWYQQKPGQPP |
| | | | | YYCSRQYGTVWFFNWGQGTLVTVSSASTKG | RLLIYAASNQGSGVPA |
| | | | | PSVFPLAPCSRSTSESTAALGCLVKDYFPEPV | RFSGSGSGTDFTLTIS |
| | | | | TVSWNSGALTSGVHTFPAVLQSSGLYSLSSVV | SLEPEDFAMYFCQQSK |
| | | | | TVPSSSLGTKTYTCNVDHKPSNTKVDKRVESK | EVPWTFGQGTKVEIKR |
| | | | | YGPPCPPCPAPEFLGGPSVFLFPPKPKDTLMI | TVAAPSVFIFPPSDEQ |
| | | | | SRTPEVTCVVVDVSQEDPEVQFNWYVDGVEV | LKSGTASVVCLLNNFY |
| | | | | HNAKTKPREEQFNSTYRVVSVLTVLHQDWLN | PREAKVQWKVDNALQS |
| | | | | GKEYKCKVSNKGLPSSIEKTISKAKGQPREPQ | GNSQESVTEQDSKDST |
| | | | | VYTLPPSQEEMTKNQVSLTCLVKGFYPSDIAV | YSLSSTLTLSKADYEK |
| | | | | EWESNGQPENNYKTTPPVLDSDGSFFLYSRL | HKVYACEVTHQGLSSP |
| | | | | TVDKSRWQEGNVFSCSVMHEALHNHYTQKSL | VTKSFNRGEC |
| | | | | SLSLGK | |
| PD-1 | geptano- | 422 | 423 | QIQLVQSGSELKKPGASVKVSCKASGYTFTNF | DIVLTQSPASLAVSPG |
| | limab | | | GMNWVRQAPGQGLKWMGWISGYTREPTYAA | QRATITCRASESVDNY |
| | | | | DFKGRFVISLDTSVSTAYLQISSLKAEDTAVYY | GYSFMNWFQQKPGQPP |
| | | | | CARDVFDYWGQGTLVTVSSASTKGPSVFPLA | KLLIYRASNLESGVPA |
| | | | | PCSRSTSESTAALGCLVKDYFPEPVTVSWNS | RFSGSGSRTDFTLTIN |
| | | | | GALTSGVHTFPAVLQSSGLYSLSSVVTVPSSS | PVEADDTANYYCQQSN |
| | | | | LGTKTYTCNVDHKPSNTKVDKRVESKYGPPC | ADPTFGQGTKLEIKRT |
| | | | | PPCPAPEFLGGPSVFLFPPKPKDTLMISRTPE | VAAPSVFIFPPSDEQL |
| | | | | VTCVVVDVSQEDPEVQFNWYVDGVEVHNAKT | KSGTASVVCLLNNFYP |
| | | | | KPREEQFNSTYRVVSVLTVLHQDWLNGKEYK | REAKVQWKVDNALQSG |
| | | | | CKVSNKGLPSSIEKTISKAKGQPREPQVYTLPP | NSQESVTEQDSKDSTY |
| | | | | SQEEMTKNQVSLTCLVKGFYPSDIAVEWESN | SLSSTLTLSKADYEKH |
| | | | | GQPENNYKTTPPVLDSDGSFFLYSRLTVDKSR | KVYACEVTHQGLSSPV |
| | | | | WQEGNVFSCSVMHEALHNHYTQKSLSLSLG | TKSFNRGEC |
| PD-1 | iparom- | 424 | 425 | QVQLVQSGAEVKKPGASVKVSCKASGYTFTN | DIQMTQSPSSLSASVG |
| | limab | | | YWIHWVRQAPGQGLEWMGEIDPYDSYTNYN | DRVTITCKSSQSLFNS |
| | | | | QKFKGRVTMTVDKSTSTVYMELSSLRSEDTA | GNQKNYLAWYQQKPGK |
| | | | | VYYCARPGFTYGGMDFWGQGTLVTVSSASTK | VPKLLIYGASTRDSGV |
| | | | | GPSVFPLAPCSRSTSESTAALGCLVKDYFPEP | PYRFSGSGSGTDFTLT |
| | | | | VTVSWNSGALTSGVHTFPAVLQSSGLYSLSSV | ISSLQPEDVATYYCQN |
| | | | | VTVPSSSLGTKTYTCNVDHKPSNTKVDKRVES | DHYYPYTFGGGTKVEI |
| | | | | KYGPPCPPCPAPEFLGGPSVFLFPPKPKDTLM | KRTVAAPSVFIFPPSD |
| | | | | ISRTPEVTCVVVDVSQEDPEVQFNWYVDGVE | EQLKSGTASVVCLLNN |
| | | | | VHNAKTKPREEQFNSTYRVVSVLTVLHQDWL | FYPREAKVQWKVDNAL |
| | | | | NGKEYKCKVSNKGLPSSIEKTISKAKGQPREP | QSGNSQESVTEQDSKD |
| | | | | QVYTLPPSQEEMTKNQVSLTCLVKGFYPSDIA | STYSLSSTLTLSKADY |
| | | | | VEWESNGQPENNYKTTPPVLDSDGSFFLYSR | EKHKVYACEVTHQGLS |
| | | | | LTVDKSRWQEGNVFSCSVMHEALHNHYTQKS | SPVTKSFNRGEC |
| | | | | LSLSLGK | |
| PD-1 | Ivonesc- | 426 | | EVQLVESGGGLVQPGGSLRLSCAASGYTFTN | |
| | imab- | | | YGMNWVRQAPGKGLEWVGWINTYTGEPTYA | |
| | bispecific | | | ADFKRRFTFSLDTSKSTAYLQMNSLRAEDTAV | |
| | anti-VEGF | | | YYCAKYPHYYGSSHWYFDVWGQGTLVTVSS | |
| | and | | | ASTKGPSVFPLAPSSKSTSGGTAALGCLVKDY | |
| | anti-PD-1 | | | FPEPVTVSWNSGALTSGVHTFPAVLQSSGLY | |
| | antibody | | | SLSSVVTVPSSSLGTQTYICNVNHKPSNTKVD | |
| | | | | KKVEPKSCDKTHTCPPCPAPEAAGGPSVFLFP | |
| | | | | PKPKDTLMISRTPEVTCVVVDVSHEDPEVKFN | |
| | | | | WYVDGVEVHNAKTKPREEQYNSTYRVVSVLT | |
| | | | | VLHQDWLNGKEYKCKVSNKALPAPIEKTISKA | |
| | | | | KGQPREPQVYTLPPSRDELTKNQVSLTCLVK | |
| | | | | GFYPSDIAVEWESNGQPENNYKTTPPVLDSD | |
| | | | | GSFFLYSKLTVDKSRWQQGNVFSCSVMHEAL | |
| | | | | HNHYTQKSLSLSPGKGGGGSGGGGSGGGGS | |
| | | | | GGGGSEVQLVESGGGLVQPGGSLRLSCAAS | |
| | | | | GFAFSSYDMSWVRQAPGKGLDWVATISGGG | |
| | | | | RYTYYPDSVKGRFTISRDNSKNNLYLQMNSLR | |
| | | | | AEDTALYYCANRYGEAWFAYWGQGTLVTVSS | |
| | | | | GGGGSGGGGSGGGGSGGGGSDIQMTQSPS | |
| | | | | SMSASVGDRVTFTCRASQDINTYLSWFQQKP | |
| | | | | GKSPKTLIYRANRLVSGVPSRFSGSGSGQDY | |
| | | | | TLTISSLQPEDMATYYCLQYDEFPLTFGAGTK | |
| | | | | LELKR | |
| PD-1 | Izurali- | 427 | | EIVLTQSPATLSASPGERVTLTCRASQSVGND | |
| | mab- | | | VAWYQQKPGQAPRLLINYASHRYTGVPDRFT | |
| | anti-PD1 | | | GSGYGTEFTLTISSVQSEDFGVYYCQQDFSSP | |
| | and | | | RTFGGGTKVEIKGKPGSGKPGSGKPGSGKPG | |
| | anti-ICOS | | | SEVQLVESGGGLVKPGGSLRLSCVASGFTFS | |
| | (anti-PD-1 | | | NYWMNWVRQAPGKGLEWVAEIRLYSNNYAT | |
| | portion on | | | HYAESVKGRFTISRDDSKSTLYLQMNNLKTED | |
| | heavy | | | TGVYYCTRYYGNYGGYFDVWGRGTLVTVSSE | |
| | chain only | | | PKSSDKTHTCPPCPAPPVAGPSVFLFPPKPKD | |
| | | | | TLMISRTPEVTCVVVDVKHEDPEVKFNWYVD | |
| | | | | GVEVHNAKTKPREEQYNSTYRVVSVLTVLHQ | |
| | | | | DWLNGKEYKCKVSNKALPAPIEKTISKAKGQP | |
| | | | | REPQVYTLPPSREQMTKNQVKLTCLVKGFYP | |
| | | | | SDIAVEWESNGQPENNYKTTPPVLDSDGSFFL | |
| | | | | YSKLTVDKSRWQQGNVFSCSVLHEALHSHYT | |
| | | | | QKSLSLSPGK | |
| PD-1 | latikafusp | 428 | 429 | EVQLVESGGSVVRPGGSLRLSCAASGFTVDD | SYELTQPPSVSVSPGQ |
| | | | | YSMSWVRQVPGKGLEWVSGINWNAGRTRYA | TARITCSGDALPKKYA |
| | | | | DAVKGRFTISRDSAKNSLYLQMNSLRAEDTAL | YWYQQKPGQAPVLVIS |
| | | | | YYCAREFNNFESNWFDPWGQGTLVTVSSAST | EDAKRPSGIPERFSGS |
| | | | | KGPSVFPLAPSSKSTSGGTAALGCLVKDYFPE | SSGTMATLTISGAQVE |
| | | | | PVTVSWNSGALTSGVHTFPAVLQSSGLYSLSS | DEADYYCYSTDASGNH |
| | | | | VVTVPSSSLGTQTYICNVNHKPSNTKVDKKVE | RVFGGGTKLTVLGQPK |
| | | | | PKSCDKTHTCPPCPAPELLGGPSVFLFPPKPK | AAPSVTLFPPSSEELQ |
| | | | | DTLMISRTPEVTCVVVDVSHEDPEVKFNWYVD | ANKATLVCLISDFYPG |
| | | | | GVEVHNAKTKPCEEQYGSTYRCVSVLTVLHQ | AVTVAWKADSSPVKAG |
| | | | | DWLNGKEYKCKVSNKALPAPIEKTISKAKGQP | VETTTPSKQSNNKYAA |
| | | | | REPQVYTLPPSRKEMTKNQVSLTCLVKGFYPS | SSYLSLTPEQWKSHRS |
| | | | | DIAVEWESNGQPENNYKTTPPVLKSDGSFFLY | YSCQVTHEGSTVEKTV |
| | | | | SKLTVDKSRWQQGNVFSCSVMHEALHNHYT | APTECS |
| | | | | QKSLSLSPGQGQDRHMIEMRQLIDIVDQLKNY | |
| | | | | VNDLVPEFLPAPEDVETNCEWSAFSCFQKAQ | |
| | | | | LKSANTGNNERIINVSIKKLKAKPPSTNAGRRQ | |
| | | | | KHRLTCPSCDSYEKKPPKEFLERFKSLLQKMI | |
| | | | | HQHLSSRTHGSEDS | |
| PD-1 | Loriger- | 430 | 431 | EIVLTQSPATLSLSPGERATLSCRASESVDNY | EIVLTQSPGTLSLSPG |
| | limab- | | | GMSFMNWFQQKPGQPPKLLIHAASNQGSGV | ERATLSCRASQSVSSS |
| | bispecific | | | PSRFSGSGSGTDFTLTISSLEPEDFAVYFCQQ | FLAWYQQKPGQAPRLL |
| | anti-PD-1 | | | SKEVPYTFGGGTKVEIKGGGSGGGGQVQLVE | IYGASSRATGIPDRFS |
| | and | | | SGGGVVQPGRSLRLSCAASGFTFSSYTMHW | GSGSGTDFTLTISRLE |
| | anti-CTLA4 | | | VRQAPGKGLEWVTFISYDGSNKHYADSVKGR | PEDFAVYYCQQYGSSP |
| | antibody | | | FTVSRDNSKNTLYLQMNSLRAEDTAIYYCART | WTFGQGTKVEIKGGGS |
| | | | | GWLGPFDYWGQGTLVTVSSGGCGGGEVAAC | GGGGQVQLVQSGAEVK |
| | | | | EKEVAALEKEVAALEKEVAALEKESKYGPPCP | KPGASVKVSCKASGYS |
| | | | | PCPAPEFLGGPSVFLFPPKPKDTLYITREPEVT | FTSYWMNWVRQAPGQG |
| | | | | CVVVDVSQEDPEVQFNWYVDGVEVHNAKTK | LEWIGVIHPSDSETWL |
| | | | | PREEQFNSTYRVVSVLTVLHQDWLNGKEYKC | DQKFKDRVTITVDKST |
| | | | | KVSNKGLPSSIEKTISKAKGQPREPQVYTLPPS | STAYMELSSLRSEDTA |
| | | | | QEEMTKNQVSLTCLVKGFYPSDIAVEWESNG | VYYCAREHYGTSPFAY |
| | | | | QPENNYKTTPPVLDSDGSFFLYSRLTVDKSR | WGQGTLVTVSSGGCGG |
| | | | | WQEGNVFSCSVMHEALHNHYTQKSLSLSLG | GKVAACKEKVAALKEK |
| | | | | | VAALKEKVAALKE |
| PD-1 | nivolumab | 432 | 433 | QVQLVESGGGVVQPGRSLRLDCKASGITFSN | EIVLTQSPATLSLSPG |
| | | | | SGMHWVRQAPGKGLEWVAVIWYDGSKRYYA | ERATLSCRASQSVSSY |
| | | | | DSVKGRFTISRDNSKNTLFLQMNSLRAEDTAV | LAWYQQKPGQAPRLLI |
| | | | | YYCATNDDYWGQGTLVTVSSASTKGPSVFPL | YDASNRATGIPARFSG |
| | | | | APCSRSTSESTAALGCLVKDYFPEPVTVSWN | SGSGTDFTLTISSLEP |
| | | | | SGALTSGVHTFPAVLQSSGLYSLSSVVTVPSS | EDFAVYYCQQSSNWPR |
| | | | | SLGTKTYTCNVDHKPSNTKVDKRVESKYGPP | TFGQGTKVEIKRTVAA |
| | | | | CPPCPAPEFLGGPSVFLFPPKPKDTLMISRTP | PSVFIFPPSDEQLKSG |
| | | | | EVTCVVVDVSQEDPEVQFNWYVDGVEVHNA | TASVVCLLNNFYPREA |
| | | | | KTKPREEQFNSTYRVVSVLTVLHQDWLNGKE | KVQWKVDNALQSGNSQ |
| | | | | YKCKVSNKGLPSSIEKTISKAKGQPREPQVYTL | ESVTEQDSKDSTYSLS |
| | | | | PPSQEEMTKNQVSLTCLVKGFYPSDIAVEWES | STLTLSKADYEKHKVY |
| | | | | NGQPENNYKTTPPVLDSDGSFFLYSRLTVDKS | ACEVTHQGLSSPVTKS |
| | | | | RWQEGNVFSCSVMHEALHNHYTQKSLSLSLG | FNRGEC |
| | | | | K | |
| PD-1 | nofazin- | 434 | 435 | QVQLVQSGAEVKKPGSSVKVSCKASGFTFTT | DVVMTQSPLSLPVTLG |
| | limab | | | YYISWVRQAPGQGLEYLGYINMGSGGTNYNE | QPASISCRSSQSLLDS |
| | | | | KFKGRVTITADKSTSTAYMELSSLRSEDTAVY | DGGTYLYWFQQRPGQS |
| | | | | YCAIIGYFDYWGQGTMVTVSSASTKGPSVFPL | PRRLIYLVSTLGSGVP |
| | | | | APCSRSTSESTAALGCLVKDYFPEPVTVSWN | DRFSGSGSGTDFTLKI |
| | | | | SGALTSGVHTFPAVLQSSGLYSLSSVVTVPSS | SRVEAEDVGVYYCMQL |
| | | | | SLGTKTYTCNVDHKPSNTKVDKRVESKYGPP | THWPYTFGQGTKLEIK |
| | | | | CPPCPAPEFLGGPSVFLFPPKPKDTLMISRTP | RTVAAPSVFIFPPSDE |
| | | | | EVTCVVVDVSQEDPEVQFNWYVDGVEVHNA | QLKSGTASVVCLLNNF |
| | | | | KTKPREEQFNSTYRVVSVLTVLHQDWLNGKE | YPREAKVQWKVDNALQ |
| | | | | YKCKVSNKGLPSSIEKTISKAKGQPREPQVYTL | SGNSQESVTEQDSKDS |
| | | | | PPSQEEMTKNQVSLTCLVKGFYPSDIAVEWES | TYSLSSTLTLSKADYE |
| | | | | NGQPENNYKTTPPVLDSDGSFFLYSRLTVDKS | KHKVYACEVTHQGLSS |
| | | | | RWQEGNVFSCSVMHEALHNHYTQKSLSLSLG | PVTKSFNRGEC |
| | | | | K | |
| PD-1 | pembro- | 436 | 437 | QVQLVQSGVEVKKPGASVKVSCKASGYTFTN | EIVLTQSPATLSLSPG |
| | lizumab | | | YYMYWVRQAPGQGLEWMGGINPSNGGTNFN | ERATLSCRASKGVSTS |
| | | | | EKFKNRVTLTTDSSTTTAYMELKSLQFDDTAV | GYSYLHWYQQKPGQAP |
| | | | | YYCARRDYRFDMGFDYWGQGTTVTVSSAST | RLLIYLASYLESGVPA |
| | | | | KGPSVFPLAPCSRSTSESTAALGCLVKDYFPE | RFSGSGSGTDFTLTIS |
| | | | | PVTVSWNSGALTSGVHTFPAVLQSSGLYSLSS | SLEPEDFAVYYCQHSR |
| | | | | VVTVPSSSLGTKTYTCNVDHKPSNTKVDKRVE | DLPLTFGGGTKVEIKR |
| | | | | SKYGPPCPPCPAPEFLGGPSVFLFPPKPKDTL | TVAAPSVFIFPPSDEQ |
| | | | | MISRTPEVTCVVVDVSQEDPEVQFNWYVDGV | LKSGTASVVCLLNNFY |
| | | | | EVHNAKTKPREEQFNSTYRVVSVLTVLHQDW | PREAKVQWKVDNALQS |
| | | | | LNGKEYKCKVSNKGLPSSIEKTISKAKGQPRE | GNSQESVTEQDSKDST |
| | | | | PQVYTLPPSQEEMTKNQVSLTCLVKGFYPSDI | YSLSSTLTLSKADYEK |
| | | | | AVEWESNGQPENNYKTTPPVLDSDGSFFLYS | HKVYACEVTHQGLSSP |
| | | | | RLTVDKSRWQEGNVFSCSVMHEALHNHYTQ | VTKSFNRGEC |
| | | | | KSLSLSLGK | |
| PD-1 | penpu- | 438 | 439 | EVQLVESGGGLVQPGGSLRLSCAASGFAFSS | DIQMTQSPSSMSASVG |
| | limab | | | YDMSWVRQAPGKGLDWVATISGGGRYTYYP | DRVTFTCRASQDINTY |
| | | | | DSVKGRFTISRDNSKNNLYLQMNSLRAEDTAL | LSWFQQKPGKSPKTLI |
| | | | | YYCANRYGEAWFAYWGQGTLVTVSSASTKG | YRANRLVSGVPSRFSG |
| | | | | PSVFPLAPSSKSTSGGTAALGCLVKDYFPEPV | SGSGQDYTLTISSLQP |
| | | | | TVSWNSGALTSGVHTFPAVLQSSGLYSLSSVV | EDMATYYCLQYDEFPL |
| | | | | TVPSSSLGTQTYICNVNHKPSNTKVDKKVEPK | TFGAGTKLELKRTVAA |
| | | | | SCDKTHTCPPCPAPEAAGAPSVFLFPPKPKDT | PSVFIFPPSDEQLKSG |
| | | | | LMISRTPEVTCVVVDVSHEDPEVKFNWYVDG | TASVVCLLNNFYPREA |
| | | | | VEVHNAKTKPREEQYNSTYRVVSVLTVLHQD | KVQWKVDNALQSGNSQ |
| | | | | WLNGKEYKCKVSNKALPAPIEKTISKAKGQPR | ESVTEQDSKDSTYSLS |
| | | | | EPQVYTLPPSRDELTKNQVSLTCLVKGFYPSD | STLTLSKADYEKHKVY |
| | | | | IAVEWESNGQPENNYKTTPPVLDSDGSFFLYS | ACEVTHQGLSSPVTKS |
| | | | | KLTVDKSRWQQGNVFSCSVMHEALHNHYTQ | FNRGEC |
| | | | | KSLSLSPGK | |
| PD-1 | pereso- | 440 | 441 | QVQLVQSGAEVKKPGASVKVSCKVSGYSLSK | DIQMTQSPSSLSASVG |
| | limab | | | YDMSWVRQAPGKGLEWMGIIYTSGYTDYAQK | DRVTITCQASQSPNNL |
| | | | | FQGRVTMTEDTSTDTAYMELSSLRSEDTAVY | LAWYQQKPGKAPKLLI |
| | | | | YCATGNPYYTNGFNSWGQGTLVTVSSASTKG | YGASDLPSGVPSRFSG |
| | | | | PSVFPLAPSSKSTSGGTAALGCLVKDYFPEPV | SGSGTDFTLTISSLQP |
| | | | | TVSWNSGALTSGVHTFPAVLQSSGLYSLSSVV | EDFATYYCQNNYYVGP |
| | | | | TVPSSSLGTQTYICNVNHKPSNTKVDKKVEPK | VSYAFGGGTKVEIKRT |
| | | | | SCDKTHTCPPCPAPELLGGPSVFLFPPKPKDT | VAAPSVFIFPPSDEQL |
| | | | | LMISRTPEVTCVVVDVSHEDPEVKFNWYVDG | KSGTASVVCLLNNFYP |
| | | | | VEVHNAKTKPREEQYNSTYRVVSVLTVLHQD | REAKVQWKVDNALQSG |
| | | | | WLNGKEYKCKVSNKALPAPIEKTISKAKGQPR | NSQESVTEQDSKDSTY |
| | | | | EPQVYTLPPSRDELTKNQVSLTCLVKGFYPSD | SLSSTLTLSKADYEKH |
| | | | | IAVEWESNGQPENNYKTTPPVLDSDGSFFLYS | KVYACEVTHQGLSSPV |
| | | | | KLTVDKSRWQQGNVFSCSVMHEALHNHYTQ | TKSFNRGEC |
| | | | | KSLSLSPGK | |
| PD-1 | pidili- | 442 | 443 | QVQLVQSGSELKKPGASVKISCKASGYTFTNY | EIVLTQSPSSLSASVG |
| | zumab | | | GMNWVRQAPGQGLQWMGWINTDSGESTYA | DRVTITCSARSSVSYM |
| | | | | EEFKGRFVFSLDTSVNTAYLQITSLTAEDTGM | HWFQQKPGKAPKLWIY |
| | | | | YFCVRVGYDALDYWGQGTLVTVSSASTKGPS | RTSNLASGVPSRFSGS |
| | | | | VFPLAPSSKSTSGGTAALGCLVKDYFPEPVTV | GSGTSYCLTINSLQPE |
| | | | | SWNSGALTSGVHTFPAVLQSSGLYSLSSVVTV | DFATYYCQQRSSFPLT |
| | | | | PSSSLGTQTYICNVNHKPSNTKVDKRVEPKSC | FGGGTKLEIKRTVAAP |
| | | | | DKTHTCPPCPAPELLGGPSVFLFPPKPKDTLM | SVFIFPPSDEQLKSGT |
| | | | | ISRTPEVTCVVVDVSHEDPEVKFNWYVDGVE | ASVVCLLNNFYPREAK |
| | | | | VHNAKTKPREEQYNSTYRVVSVLTVLHQDWL | VQWKVDNALQSGNSQE |
| | | | | NGKEYKCKVSNKALPAPIEKTISKAKGQPREP | SVTEQDSKDSTYSLSS |
| | | | | QVYTLPPSREEMTKNQVSLTCLVKGFYPSDIA | TLTLSKADYEKHKVYA |
| | | | | VEWESNGQPENNYKTTPPVLDSDGSFFLYSK | CEVTHQGLSSPVTKSF |
| | | | | LTVDKSRWQQGNVFSCSVMHEALHNHYTQK | NRGEC |
| | | | | SLSLSPGK | |
| PD-1 | pimiva- | 444 | 445 | QVQLVQSGAEVKKPGASVKVSCKASGYTFPS | DIQMTQSPSTLSASVG |
| | limab | | | YYMHWVRQAPGQGLEWMGIINPEGGSTAYA | DRVTITCRASQSISSW |
| | | | | QKFQGRVTMTRDTSTSTVYMELSSLRSEDTA | LAWYQQKPGKAPKLLI |
| | | | | VYYCARGGTYYDYTYWGQGTLVTVSSASTKG | YEASSLESGVPSRFSG |
| | | | | PSVFPLAPCSRSTSESTAALGCLVKDYFPEPV | SGSGTEFTLTISSLQP |
| | | | | TVSWNSGALTSGVHTFPAVLQSSGLYSLSSVV | DDFATYYCQQYNSFPP |
| | | | | TVPSSSLGTKTYTCNVDHKPSNTKVDKRVESK | TFGGGTKVEIKRTVAA |
| | | | | YGPPCPPCPAPEFLGGPSVFLFPPKPKDTLMI | PSVFIFPPSDEQLKSG |
| | | | | SRTPEVTCVVVDVSQEDPEVQFNWYVDGVEV | TASVVCLLNNFYPREA |
| | | | | HNAKTKPREEQFNSTYRVVSVLTVLHQDWLN | KVQWKVDNALQSGNSQ |
| | | | | GKEYKCKVSNKGLPSSIEKTISKAKGQPREPQ | ESVTEQDSKDSTYSLS |
| | | | | VYTLPPSQEEMTKNQVSLTCLVKGFYPSDIAV | STLTLSKADYEKHKVY |
| | | | | EWESNGQPENNYKTTPPVLDSDGSFFLYSRL | ACEVTHQGLSSPVTKS |
| | | | | TVDKSRWQEGNVFSCSVMHEALHNHYTQKSL | FNRGEC |
| | | | | SLSLGK | |
| PD-1 | prolgo- | 446 | 447 | QVQLVQSGGGLVQPGGSLRLSCAASGFTFSS | QPVLTQPLSVSVALGQ |
| | limab | | | YWMYWVRQVPGKGLEWVSAIDTGGGRTYYA | TARITCGGNNIGSKNV |
| | | | | DSVKGRFAISRVNAKNTMYLQMNSLRAEDTA | HWYQQKPGQAPVLVIY |
| | | | | VYYCARDEGGGTGWGVLKDWPYGLDAWGQ | RDSNRPSGIPERFSGS |
| | | | | GTLVTVSSASTKGPSVFPLAPSSKSTSGGTAA | NSGNTATLTISRAQAG |
| | | | | LGCLVKDYFPEPVTVSWNSGALTSGVHTFPA | DEADYYCQVWDSSTAV |
| | | | | VLQSSGLYSLSSVVTVPSSSLGTQTYICNVNH | FGTGTKLTVLQRTVAA |
| | | | | KPSNTKVDKRVEPKSCDKTHTCPPCPAPEAA | PSVFIFPPSDEQLKSG |
| | | | | GGPSVFLFPPKPKDTLMISRTPEVTCVVVDVS | TASVVCLLNNFYPREA |
| | | | | HEDPEVKFNWYVDGVEVHNAKTKPREEQYNS | KVQWKVDNALQSGNSQ |
| | | | | TYRVVSVLTVLHQDWLNGKEYKCKVSNKALP | ESVTEQDSKDSTYSLS |
| | | | | APIEKTISKAKGQPREPQVYTLPPSREEMTKN | STLTLSKADYEKHKVY |
| | | | | QVSLTCLVKGFYPSDIAVEWESNGQPENNYK | ACEVTHQGLSSPVTKS |
| | | | | TTPPVLDSDGSFFLYSKLTVDKSRWQQGNVF | FNRGEC |
| | | | | SCSVMHEALHNHYTQKSLSLSPGK | |
| PD-1 | pucoten- | 448 | 449 | EVQLVQSGGGLVQPGGSLKLSCAASGFTFSS | DIVLTQSPASLAVSPG |
| | limab | | | YGMSWVRQAPGKGLDWVATISGGGRDTYYP | QRATITCRASESVDNY |
| | | | | DSVKGRFTISRDNSKNNLYLQMNSLRAEDTAL | GISFMNWFQQKPGQPP |
| | | | | YYCARQKGEAWFAYWGQGTLVTVSSASTKG | KLLIYAASNKGTGVPA |
| | | | | PSVFPLAPCSRSTSESTAALGCLVKDYFPEPV | RFSGSGSGTDFTLNIN |
| | | | | TVSWNSGALTSGVHTFPAVLQSSGLYSLSSVV | PMEENDTAMYFCQQSK |
| | | | | TVPSSSLGTKTYTCNVDHKPSNTKVDKRVESK | EVPWTFGGGTKLEIKR |
| | | | | YGPPCPPCPAPEFLGGPSVFLFPPKPKDTLMI | TVAAPSVFIFPPSDEQ |
| | | | | TRTPEVTCVVVDVSQEDPEVQFNWYVDGVEV | LKSGTASVVCLLNNFY |
| | | | | HNAKTKPREEQFNSTYRVVSVLTPLHQDWLN | PREAKVQWKVDNALQS |
| | | | | GKEYKCKVSNKGLPSSIEKTISKAKGQPREPQ | GNSQESVTEQDSKDST |
| | | | | VYTLPPSQEEMTKNQVSLTCLVKGFYPSDIAV | YSLSSTLTLSKADYEK |
| | | | | EWESNGQPENNYKTTPPVLDSDGSFFLYSRL | HKVYACEVTHQGLSSP |
| | | | | TVDKSRWQEGNVFSCSVMHEALHAHYTQKSL | VTKSFNRGEC |
| | | | | SLSLGK | |
| PD-1 | reozalimab | 450 | 451 | QVQLVQSGAEVKKPGSSVKVSCKASGGTFSS | DIQMTQSPSSVSASVG |
| | | | | YAISWVRQAPGQGLEWMGLIIPSFDTAGYAQK | DRVTITCRASQGISSW |
| | | | | FQGRVAITVDESTSTAYMELSSLRSEDTAVYY | LAWYQQKPGKAPKLLI |
| | | | | CARAEHSSTGTFDYWGQGTLVTVSSASTKGP | SAASSLQSGVPSRFSG |
| | | | | SVFPEAPSSKSTSGGTAALGCLVTDYFPEPVT | SGSGTDFTLTISSLQP |
| | | | | VSWNSGALTSGVHTFPAVLESSGLYSLSSVVT | EDFATYYCQQANHLPF |
| | | | | VPSSSLGTQTYICNVNHKPSNTKVDKRVEPKS | TFGGGTKVEIKRTVAA |
| | | | | CDKTHTCPPCPAPEAAGGPSVFLFPPKPKDTL | PSVFIFPPSDEQLKSG |
| | | | | MISRTPEVTCVVVSVSHEDPEVKFNWYVDGV | TARVGCLLNNFYPREA |
| | | | | EVHNAKTKPREEQYNSTYRVVSVLTVLHQDW | KVQWKVDNALQSGNSQ |
| | | | | LNGKEYKCKVSNKALPAPIEKTISKAKGQPREP | ESVTEQDSKDSTYSLR |
| | | | | QVYVLPPSRDELTKNQVSLLCLVKGFYPSDIA | STLTLSKADYEKHKVY |
| | | | | VEWESNGQPENNYLTWPPVLDSDGSFFLYSK | ACEVTHQGLSSPVTKS |
| | | | | LTVDKSRWQQGNVFSCSVMHEALHNHYTQK | FNRGEC |
| | | | | SLSLSPGK | |
| PD-1 | retifan- | 452 | 453 | QVQLVQSGAEVKKPGASVKVSCKASGYSFTS | EIVLTQSPATLSLSPG |
| | limab | | | YWMNWVRQAPGQGLEWIGVIHPSDSETWLD | ERATLSCRASESVDNY |
| | | | | QKFKDRVTITVDKSTSTAYMELSSLRSEDTAV | GMSFMNWFQQKPGQPP |
| | | | | YYCAREHYGTSPFAYWGQGTLVTVSSASTKG | KLLIHAASNQGSGVPS |
| | | | | PSVFPLAPCSRSTSESTAALGCLVKDYFPEPV | RFSGSGSGTDFTLTIS |
| | | | | TVSWNSGALTSGVHTFPAVLQSSGLYSLSSVV | SLEPEDFAVYFCQQSK |
| | | | | TVPSSSLGTKTYTCNVDHKPSNTKVDKRVESK | EVPYTFGGGTKVEIKR |
| | | | | YGPPCPPCPAPEFLGGPSVFLFPPKPKDTLMI | TVAAPSVFIFPPSDEQ |
| | | | | SRTPEVTCVVVDVSQEDPEVQFNWYVDGVEV | LKSGTASVVCLLNNFY |
| | | | | HNAKTKPREEQFNSTYRVVSVLTVLHQDWLN | PREAKVQWKVDNALQS |
| | | | | GKEYKCKVSNKGLPSSIEKTISKAKGQPREPQ | GNSQESVTEQDSKDST |
| | | | | VYTLPPSQEEMTKNQVSLTCLVKGFYPSDIAV | YSLSSTLTLSKADYEK |
| | | | | EWESNGQPENNYKTTPPVLDSDGSFFLYSRL | HKVYACEVTHQGLSS |
| | | | | TVDKSRWQEGNVFSCSVMHEALHNHYTQKSL | PVTKSFNRGEC |
| | | | | SLSLG | |
| PD-1 | rosnilimab | 454 | 455 | QVQLVQSGSELKKPGASVKVSCKASNYTFTD | EIVLTQSPATLSLSPG |
| | | | | YSMHWVRQAPGQGLEWMGWINIETYYPTYA | ERATLSCTASSSVSSS |
| | | | | DQFKGRFAFSLDTSVSTAYLQISSLKAEDTAVY | YFHWYQQKPGQAPRLL |
| | | | | YCARDYYGRFYYAMDYWGQGTTVTVSSAST | IYSTSNLASGIPARFS |
| | | | | KGPSVFPLAPSSKSTSGGTAALGCLVKDYFPE | GSGSGTDFTLTISRLE |
| | | | | PVTVSWNSGALTSGVHTFPAVLQSSGLYSLSS | PEDFAVYYCHQYHRSP |
| | | | | VVTVPSSSLGTQTYICNVNHKPSNTKVDKKVE | LTFGGGTKVEIKRTVA |
| | | | | PKSCDKTHTCPPCPAPELLGGPSVFLFPPKPK | APSVFIFPPSDEQLKS |
| | | | | DTLMISRTPEVTCVVVDVSHEDPEVKFNWYVD | GTASVVCLLNNFYPRE |
| | | | | GVEVHNAKTKPREEQYNSTYRVVSVLTVLHQ | AKVQWKVDNALQSGNS |
| | | | | DWLNGKEYKCKVSNKALPAPIEKTISKAKGQP | QESVTEQDSKDSTYSL |
| | | | | REPQVYTLPPSRDELTKNQVSLTCLVKGFYPS | SSTLTLSKADYEKHKV |
| | | | | DIAVEWESNGQPENNYKTTPPVLDSDGSFFLY | YACEVTHQGLSSPVTK |
| | | | | SKLTVDKSRWQQGNVFSCSVMHEALHNHYT | SFNRGEC |
| | | | | QKSLSLSPGK | |
| PD-1 | ruloni- | 456 | 457 | EVQLVESGGGLVKPGGSLRLSCAASGFTFSS | EIVLTQSPATLAVSPG |
| | limab | | | YGMSWVRQTPEKRLEWVATISGGGRDTYYP | ERATISCRASESVDDY |
| | | | | DSVKGRFTISRDNAKNNLYLQMSSLRSEDTAL | GISFMNWFQQKPGQPP |
| | | | | YYCARQKDTSWFVHWGQGTLVTVSSASTKG | KLLIYVASNQGSGVPA |
| | | | | PSVFPLAPSSKSTSGGTAALGCLVKDYFPEPV | RFSGSGSGTDFTLNIH |
| | | | | TVSWNSGALTSGVHTFPAVLQSSGLYSLSSVV | PMEEDDTAMYFCQQSK |
| | | | | TVPSSSLGTQTYICNVNHKPSNTKVDKKVEPK | EVPWTFGGGTKLEIKR |
| | | | | SCDKTHTCPPCPAPELLGGPSVFLFPPKPKDQ | TVAAPSVFIFPPSDEQ |
| | | | | LMISRTPEVTCVVVDVSHEDPEVKFNWYVDG | LKSGTASVVCLLNNFY |
| | | | | VEVHNAKTKPREEQYASTYRVVSVLTVLHQD | PREAKVQWKVDNALQS |
| | | | | WLNGKEYKCKVSNKALPAPIEKTISKAKGQPR | GNSQESVTEQDSKDST |
| | | | | EPQVYTLPPSREEMTKNQVSLTCLVKGFYPSD | YSLSSTLTLSKADYEK |
| | | | | IAVEWESNGQPENNYKTTPPVLDSDGSFFLYS | HKVYACEVTHQGLSSP |
| | | | | KLTVDKSRWQQGNVFSCSVLHEALHNHYTQK | VTKSFNRGEC |
| | | | | SLSLSPGK | |
| PD-1 | sasan- | 458 | 459 | QVQLVQSGAEVKKPGASVKVSCKASGYTFTS | DIVMTQSPDSLAVSLG |
| | limab | | | YWINWVRQAPGQGLEWMGNIYPGSSLTNYN | ERATINCKSSQSLWDS |
| | | | | EKFKNRVTMTRDTSTSTVYMELSSLRSEDTAV | GNQKNFLTWYQQKPGQ |
| | | | | YYCARLSTGTFAYWGQGTLVTVSSASTKGPS | PPKLLIYWTSYRESGV |
| | | | | VFPLAPCSRSTSESTAALGCLVKDYFPEPVTV | PDRFSGSGSGTDFTLT |
| | | | | SWNSGALTSGVHTFPAVLQSSGLYSLSSVVTV | ISSLQAEDVAVYYCQN |
| | | | | PSSSLGTKTYTCNVDHKPSNTKVDKRVESKY | DYFYPHTFGGGTKVEI |
| | | | | GPPCPPCPAPEFLGGPSVFLFPPKPKDTLMIS | KRTVAAPSVFIFPPSD |
| | | | | RTPEVTCVVVDVSQEDPEVQFNWYVDGVEVH | EQLKSGTASVVCLLNN |
| | | | | NAKTKPREEQFNSTYRVVSVLTVLHQDWLNG | FYPREAKVQWKVDNAL |
| | | | | KEYKCKVSNKGLPSSIEKTISKAKGQPREPQV | QSGNSQESVTEQDSKD |
| | | | | YTLPPSQEEMTKNQVSLTCLVKGFYPSDIAVE | STYSLSSTLTLSKADY |
| | | | | WESNGQPENNYKTTPPVLDSDGSFFLYSRLT | EKHKVYACEVTHQGLS |
| | | | | VDKSRWQEGNVFSCSVMHEALHNHYTQKSL | SPVTKSFNRGEC |
| | | | | SLSLGK | |
| PD-1 | serplu- | 460 | 461 | QVQLVESGGGLVKPGGSLRLSCAASGFTFSN | DIQMTQSPSSLSASVG |
| | limab | | | YGMSWIRQAPGKGLEWVSTISGGGSNIYYAD | DRVTITCKASQDVTTA |
| | | | | SVKGRFTISRDNAKNSLYQMNSLRAEDTAVY | VAWYQQKPGKAPKLLI |
| | | | | YCVSYYYGIDFWGQGTSVTVSSASTKGPSVF | YWASTRHTGVPSRFSG |
| | | | | PLAPCSRSTSESTAALGCLVKDYFPEPVTVSW | SGSGTDFTLTISSLQP |
| | | | | NSGALTSGVHTFPAVLQSSGLYSLSSVVTVPS | EDFATYYCQQHYTIPW |
| | | | | SSLGTKTYTCNVDHKPSNTKVDKRVESKYGP | TFGGGTKLEIKRTVAA |
| | | | | PCPPCPAPEFLGGPSVFLFPPKPKDTLMISRT | PSVFIFPPSDEQLKSG |
| | | | | PEVTCVVVDVSQEDPEVQFNWYVDGVEVHN | TASVVCLLNNFYPREA |
| | | | | AKTKPREEQFNSTYRVVSVLTVLHQDWLNGK | KVQWKVDNALQSGNSQ |
| | | | | EYKCKVSNKGLPSSIEKTISKAKGQPREPQVY | ESVTEQDSKDSTYSLS |
| | | | | TLPPSQEEMTKNQVSLTCLVKGFYPSDIAVEW | STLTLSKADYEKHKVY |
| | | | | ESNGQPENNYKTTPPVLDSDGSFFLYSRLTVD | ACEVTHQGLSSPVTKS |
| | | | | KSRWQEGNVFSCSVMHEALHNHYTQKSLSLS | FNRGEC |
| | | | | LGK | |
| PD-1 | sintilimab | 462 | 463 | QVQLVQSGAEVKKPGSSVKVSCKASGGTFSS | DIQMTQSPSSVSASVG |
| | | | | YAISWVRQAPGQGLEWMGLIIPMFDTAGYAQ | DRVTITCRASQGISSW |
| | | | | KFQGRVAITVDESTSTAYMELSSLRSEDTAVY | LAWYQQKPGKAPKLLI |
| | | | | YCARAEHSSTGTFDYWGQGTLVTVSSASTKG | SAASSLQSGVPSRFSG |
| | | | | PSVFPLAPCSRSTSESTAALGCLVKDYFPEPV | SGSGTDFTLTISSLQP |
| | | | | TVSWNSGALTSGVHTFPAVLQSSGLYSLSSVV | EDFATYYCQQANHLPF |
| | | | | TVPSSSLGTKTYTCNVDHKPSNTKVDKRVESK | TFGGGTKVEIKRTVAA |
| | | | | YGPPCPPCPAPEFLGGPSVFLFPPKPKDTLMI | PSVFIFPPSDEQLKSG |
| | | | | SRTPEVTCVVVDVSQEDPEVQFNWYVDGVEV | TASVVCLLNNFYPREA |
| | | | | HNAKTKPREEQFNSTYRVVSVLTVLHQDWLN | KVQWKVDNALQSGNSQ |
| | | | | GKEYKCKVSNKGLPSSIEKTISKAKGQPREPQ | ESVTEQDSKDSTYSLS |
| | | | | VYTLPPSQEEMTKNQVSLTCLVKGFYPSDIAV | STLTLSKADYEKHKVY |
| | | | | EWESNGQPENNYKTTPPVLDSDGSFFLYSRL | ACEVTHQGLSSPVTKS |
| | | | | TVDKSRWQEGNVFSCSVMHEALHNHYTQKSL | FNRGEC |
| | | | | SLSLGK | |
| PD-1 | spartal- | 464 | 465 | EVQLVQSGAEVKKPGESLRISCKGSGYTFTTY | EIVLTQSPATLSLSPG |
| | izumab | | | WMHWVRQATGQGLEWMGNIYPGTGGSNFD | ERATLSCKSSQSLLDS |
| | | | | EKFKNRVTITADKSTSTAYMELSSLRSEDTAVY | GNQKNFLTWYQQKPGQ |
| | | | | YCTRWTTGTGAYWGQGTTVTVSSASTKGPS | APRLLIYWASTRESGV |
| | | | | VFPLAPCSRSTSESTAALGCLVKDYFPEPVTV | PSRFSGSGSGTDFTFT |
| | | | | SWNSGALTSGVHTFPAVLQSSGLYSLSSVVTV | ISSLEAEDAATYYCQN |
| | | | | PSSSLGTKTYTCNVDHKPSNTKVDKRVESKY | DYSYPYTFGQGTKVEI |
| | | | | GPPCPPCPAPEFLGGPSVFLFPPKPKDTLMIS | KRTVAAPSVFIFPPSD |
| | | | | RTPEVTCVVVDVSQEDPEVQFNWYVDGVEVH | EQLKSGTASVVCLLNN |
| | | | | NAKTKPREEQFNSTYRVVSVLTVLHQDWLNG | FYPREAKVQWKVDNAL |
| | | | | KEYKCKVSNKGLPSSIEKTISKAKGQPREPQV | QSGNSQESVTEQDSKD |
| | | | | YTLPPSQEEMTKNQVSLTCLVKGFYPSDIAVE | STYSLSSTLTLSKADY |
| | | | | WESNGQPENNYKTTPPVLDSDGSFFLYSRLT | EKHKVYACEVTHQGLS |
| | | | | VDKSRWQEGNVFSCSVMHEALHNHYTQKSL | SPVTKSFNRGEC |
| | | | | SLSLG | |
| PD-1 | sudubril- | 466 | 467 | EVQLVESGGGLVQPGGSLRLSCAASGFTFSE | DIQMTQSPSSLSASVG |
| | imab | | | TWLHWVRQAPGKGLEWVAWVSPFGGSTYYA | DRVTITCRASQDVSTA |
| | | | | DSVKGRFTISADTSKNTAYLQMNSLRAEDTAV | VAWYQQKPGKAPKLLI |
| | | | | YYCARRHWPGGFDYWGQGTLVTVSSASTKG | YSASFLYSGVPSRFSG |
| | | | | PSVFPLAPSSKSTSGGTAALGCLVKDYFPEPV | SGSGTDFTLTISSLQP |
| | | | | TVSWNSGALTSGVHTFPAVLQSSGLYSLSSVV | EDFATYYCQQFLYHPA |
| | | | | TVPSSSLGTQTYICNVNHKPSNTKVDKKVEPK | TFGQGTKVEIKRTVAA |
| | | | | SCDKTHTCPPCPAPELLGGPSVFLFPPKPKDT | PSVFIFPPSDEQLKSG |
| | | | | LMISRTPEVTCVVVDVSHEDPEVKFNWYVDG | TASVVCLLNNFYPREA |
| | | | | VEVHNAKTKPREEQYASTYRVVSVLTVLHQD | KVQWKVDNALQSGNSQ |
| | | | | WLNGKEYKCKVSNKALPAPIEKTISKAKGQPR | ESVTEQDSKDSTYSLS |
| | | | | EPQVYTLPPSREEMTKNQVSLTCLVKGFYPSD | STLTLSKADYEKHKVY |
| | | | | IAVEWESNGQPENNYKTTPPVLDSDGSFFLYS | ACEVTHQGLSSPVTKS |
| | | | | KLTVDKSRWQQGNVFSCSVMHEALHNHYTQ | FNRGEC |
| | | | | KSLSLSPG | |
| PD-1 | tisleli- | 468 | 469 | QVQLQESGPGLVKPSETLSLTCTVSGFSLTSY | DIVMTQSPDSLAVSLG |
| | zumab | | | GVHWIRQPPGKGLEWIGVIYADGSTNYNPSLK | ERATINCKSSESVSND |
| | | | | SRVTISKDTSKNQVSLKSSVTAADTAVYYCA | VAWYQQKPGQPPKLLI |
| | | | | RAYGNYWYIDVWGQGTTVTVSSASTKGPSVF | NYAFHRFTGVPDRFSG |
| | | | | PLAPCSRSTSESTAALGCLVKDYFPEPVTVSW | SGYGTDFTLTISSLQA |
| | | | | NSGALTSGVHTFPAVLQSSGLYSLSSVVTVPS | EDVAVYYCHQAYSSPY |
| | | | | SSLGTKTYTCNVDHKPSNTKVDKRVESKYGP | TFGQGTKLEIKRTVAA |
| | | | | PCPPCPAPPVAGGPSVFLFPPKPKDTLMISRT | PSVFIFPPSDEQLKSG |
| | | | | PEVTCVVVAVSQEDPEVQFNWYVDGVEVHNA | TASVVCLLNNFYPREA |
| | | | | KTKPREEQFNSTYRVVSVLTVVHQDWLNGKE | KVQWKVDNALQSGNSQ |
| | | | | YKCKVSNKGLPSSIEKTISKAKGQPREPQVYTL | ESVTEQDSKDSTYSLS |
| | | | | PPSQEEMTKNQVSLTCLVKGFYPSDIAVEWES | STLTLSKADYEKHKVY |
| | | | | NGQPENNYKTTPPVLDSDGSFFLYSKLTVDKS | ACEVTHQGLSSPVTKS |
| | | | | RWQEGNVFSCSVMHEALHNHYTQKSLSLSLG | FNRGEC |
| | | | | K | |
| PD-1 | toripal- | 470 | 471 | QGQLVQSGAEVKKPGASVKVSCKASGYTFTD | DVVMTQSPLSLPVTLG |
| | imab | | | YEMHWVRQAPIHGLEWIGVIESETGGTAYNQ | QPASISCRSSQSIVHS |
| | | | | KFKGRVTITADKSTSTAYMELSSLRSEDTAVY | NGNTYLEWYLQKPGQS |
| | | | | YCAREGITTVATTYYWYFDVWGQGTTVTVSS | PQLLIYKVSNRFSGVP |
| | | | | ASTKGPSVFPLAPCSRSTSESTAALGCLVKDY | DRFSGSGSGTDFTLKI |
| | | | | FPEPVTVSWNSGALTSGVHTFPAVLQSSGLY | SRVEAEDVGVYYCFQG |
| | | | | SLSSVVTVPSSSLGTKTYTCNVDHKPSNTKVD | SHVPLTFGQGTKLEIK |
| | | | | KRVESKYGPPCPPCPAPEFLGGPSVFLFPPKP | RTVAAPSVFIFPPSDE |
| | | | | KDTLMISRTPEVTCVVVDVSQEDPEVQFNWY | QLKSGTASVVCLLNNF |
| | | | | VDGVEVHNAKTKPREEQFNSTYRVVSVLTVLH | YPREAKVQWKVDNALQ |
| | | | | QDWLNGKEYKCKVSNKGLPSSIEKTISKAKGQ | SGNSQESVTEQDSKDS |
| | | | | PREPQVYTLPPSQEEMTKNQVSLTCLVKGFY | TYSLSSTLTLSKADYE |
| | | | | PSDIAVEWESNGQPENNYKTTPPVLDSDGSF | KHKVYACEVTHQGLSS |
| | | | | FLYSRLTVDKSRWQEGNVFSCSVMHEALHNH | PVTKSFNRGEC |
| | | | | YTQKSLSLSLGK | |
| PD-1 | vudalimab | 472 | 473 | EVQLVESGGGLVKPGGSLRLSCAASGFTFSS | EIVLTQSPGTLSLSPG |
| | | | | YTMHWVRQAPGKGLEWVSFISYDGNNKYYA | ERATLSCRASQSVSSS |
| | | | | DSVKGRFTISRDNAKNSLYLQMNSLRAEDTAV | YLAWYQQKPGQAPRLL |
| | | | | YYCARTGWLGPFDYWGQGTLVTVSSASTKG | IYGAFSRATGIPDRFS |
| | | | | PSVFPLAPSSKSTSGGTAALGCLVKDYFPEPV | GSGSGTDFTLTISRLE |
| | | | | TVSWNSGALTSGVHTFPAVLQSSGLYSLSSVV | PEDFAVYYCQQYGSSP |
| | | | | TVPSSSLGTQTYICNVNHKPSDTKVDKKVEPK | WTFGQGTKVEIKRTVA |
| | | | | SCDKTHTCPPCPAPPVAGPSVFLFPPKPKDTL | APSVFIFPPSDEQLKS |
| | | | | MISRTPEVTCVVVDVKHEDPEVKFNWYVDGV | GTASVVCLLNNFYPRE |
| | | | | EVHNAKTKPREEEYNSTYRVVSVLTVLHQDW | AKVQWKVDNALQSGNS |
| | | | | LNGKEYKCKVSNKALPAPIEKTISKAKGQPREP | QESVTEQDSKDSTYSL |
| | | | | QVYTLPPSREEMTKNQVSLTCDVSGFYPSDIA | SSTLTLSKADYEKHKV |
| | | | | VEWESDGQPENNYKTTPPVLDSDGSFFLYSK | YACEVTHQGLSSPVTK |
| | | | | LTVDKSRWEQGDVFSCSVLHEALHSHYTQKS | SFNRGEC |
| | | | | LSLSPGK | |
| PD-1 | zeluva- | 474 | 475 | EVQLLESGGGLVQPGGSLRLSCAASGFTFSS | DIQMTQSPSSVSASVG |
| | limab | | | YDMSWVRQAPGKGLEWVSLISGGGSQTYYA | DRVTITCRASQGISNW |
| | | | | ESVKGRFTISRDNSKNTLYLQMNSLRAEDTAV | LAWYQQKPGKAPKLLI |
| | | | | YFCASPSGHYFYAMDVWGQGTTVTVSSASTK | FAASSLQSGVPSRFSG |
| | | | | GPSVFPLAPSSKSTSGGTAALGCLVKDYFPEP | SGSGTDFTLTISSLQP |
| | | | | VTVSWNSGALTSGVHTFPAVLQSSGLYSLSSV | EDFATYYCQQAESFPH |
| | | | | VTVPSSSLGTQTYICNVNHKPSNTKVDKKVEP | TFGGGTKVEIKRTVAA |
| | | | | KSCDKTHTCPPCPAPELLGGPSVFLFPPKPKD | PSVFIFPPSDEQLKSG |
| | | | | TLMISRTPEVTCVVVDVSHEDPEVKFNWYVD | TASVVCLLNNFYPREA |
| | | | | GVEVHNAKTKPCEEQYGSTYRCVSVLTVLHQ | KVQWKVDNALQSGNSQ |
| | | | | DWLNGKEYKCKVSNKALPAPIEKTISKAKGQP | ESVTEQDSKDSTYSLS |
| | | | | REPQVYTLPPSREEMTKNQVSLTCLVKGFYPS | STLTLSKADYEKHKVY |
| | | | | DIAVEWESNGQPENNYKTTPPVLDSDGSFFLY | ACEVTHQGLSSPVTKS |
| | | | | SKLTVDKSRWQQGNVFSCSVMHEALHNHYT | FNRGEC |
| | | | | QKSLSLSPGK | |
| PD-1 | zimbere- | 476 | 477 | QLQLQESGPGLVKPSETLTLTCTVSADSISSTT | QSALTQPASVSGSPGQ |
| | limab | | | YYWVWIRQPPGKGLEWIGSISYSGSTYYNPSL | SITISCTGTSSDVGFY |
| | | | | KSRVTVSVDTSKNQFSLKLNSVAATDTALYYC | NYVSWYQQHPGKAPEL |
| | | | | ARHLGYNGRYLPFDYWGQGTLVTVSSASTKG | MIYDVSNRPSGVSDRF |
| | | | | PSVFPLAPCSRSTSESTAALGCLVKDYFPEPV | SGSKSGNTASLTISGL |
| | | | | TVSWNSGALTSGVHTFPAVLQSSGLYSLSSVV | QAEDEADYYCSSYTSI |
| | | | | TVPSSSLGTKTYTCNVDHKPSNTKVDKRVESK | STWVFGGGTKLTVLGQ |
| | | | | YGPPCPPCPAPEFLGGPSVFLFPPKPKDTLMI | PKAAPSVTLFPPSSEE |
| | | | | SRTPEVTCVVVDVSQEDPEVQFNWYVDGVEV | LQANKATLVCLISDFY |
| | | | | HNAKTKPREEQFNSTYRVVSVLTVLHQDWLN | PGAVTVAWKADSSPVK |
| | | | | GKEYKCKVSNKGLPSSIEKTISKAKGQPREPQ | AGVETTTPSKQSNNKY |
| | | | | VYTLPPSQEEMTKNQVSLTCLVKGFYPSDIAV | AASSYLSLTPEQWKSH |
| | | | | EWESNGQPENNYKTTPPVLDSDGSFFLYSRL | RSYSCQVTHEGSTVEK |
| | | | | TVDKSRWQEGNVFSCSVMHEALHNHYTQKSL | TVAPTECS |
| | | | | SLSLGK | |
| PD-1 | tebote- | 478 | 479 | DIQMTQSPSSLSASVGDRVTITCRASQDVSSV | EIVLTQSPATLSLSPG |
| | limab | | | VAWYQQKPGKAPKLLIYSASYRYTGVPSRFS | ERATLSCRASESVDNY |
| | | | | GSGSGTDFTLTISSLQPEDFATYYCQQHYSTP | GMSFMNWFQQKPGQPP |
| | | | | WTFGGGTKLEIKGGGSGGGGQVQLVQSGAE | KLLIHAASNQGSGVPS |
| | | | | VKKPGASVKVSCKASGYSFTSYWMNWVRQA | RFSGSGSGTDFTLTIS |
| | | | | PGQGLEWIGVIHPSDSETWLDQKFKDRVTITV | SLEPEDFAVYFCQQSK |
| | | | | DKSTSTAYMELSSLRSEDTAVYYCAREHYGTS | EVPYTFGGGTKVEIKG |
| | | | | PFAYWGQGTLVTVSSGGGGGGEVAACEKEV | GGSGGGGQVQLVQSGA |
| | | | | AALEKEVAALEKEVAALEKESKYGPPCPPCPA | EVKKPGASVKVSCKAS |
| | | | | PEFLGGPSVFLFPPKPKDTLYITREPEVTCVVV | GYTFTDYNMDWVRQAP |
| | | | | DVSQEDPEVQFNWYVDGVEVHNAKTKPREE | GQGLEWMGDINPDNGV |
| | | | | QFNSTYRVVSVLTVLHQDWLNGKEYKCKVSN | TIYNQKFEGRVTMTTD |
| | | | | KGLPSSIEKTISKAKGQPREPQVYTLPPSQEE | TSTSTAYMELRSLRSD |
| | | | | MTKNQVSLTCLVKGFYPSDIAVEWESNGQPE | DTAVYYCAREADYFYF |
| | | | | NNYKTTPPVLDSDGSFFLYSRLTVDKSRWQE | DYWGQGTTLTVSSGGG |
| | | | | GNVFSCSVMHEALHNHYTQKSLSLSLG | GGKVAACKEKVAALKE |
| | | | | | KVAALKEKVAALKE |
| Siglec- | AB-25E9 | 480 | 481 | EIQLQQSGVELVRPGASVTLSCKASGYTFTDY | DIVMTQAAPSVPVTPG |
| 15 | | | | DMHWVKQTPVHGLEWIGTIDPETGGTAYNQK | ESVSISCRSTKSLLHS |
| | | | | FKGKATLTADRSSTTAYMELSSLTSEDSAVYY | NGNTYLYWFLQRPGQS |
| | | | | CTSFYYTYSNYDVGFAYWGQGTLVTVSA | PQLLIYRMSNLASGVP |
| | | | | | DRFSGSGSGTAFTLRI |
| | | | | | SRVEAEDVGVYYCMQH |
| | | | | | LEYPFTFGGGTKLEIK |
| Siglec- | A9E8 | 482 | 483 | QLQLQESGPGLVKPSETLSLTCAVSGASISNW | SSELTQDPAVSVALGQ |
| 15 | | | | WSWVRQPPGKGLEWIGEVHHSGVTTYKPSLK | TVRITCRGDSLRKYYA |
| | | | | SRVTISVDNSKNQLSKLTSVTAADTAVYYCAR | SWYQQKPRQAPQLVIY |
| | | | | EFADDAFDIWGRGTMVTVSS | HKNNRASGIPDRFSGS |
| | | | | | ISGNTASLTITGAQAE |
| | | | | | DEAAYFCNSRDTSGNY |
| | | | | | LVFGGGTKVTVLG |
| TIGIT | tirago- | 484 | 485 | EVQLQQSGPGLVKPSQTLSLTCAISGDSVSSN | DIVMTQSPDSLAVSLG |
| | lumab | | | SAAWMWIRQSPSRGLEWLGKTYYRFKWYSD | ERATINCKSSQTVLYS |
| | | | | YAVSVKGRITINPDTSKNQFSLQLNSVTPEDTA | SNNKKYLAWYQQKPGQ |
| | | | | VFYCTRESTTYDLLAGPFDYWGQGTLVTVSS | PPNLLIYWASTRESGV |
| | | | | ASTKGPSVFPLAPSSKSTSGGTAALGCLVKDY | PDRFSGSGSGTDFTLT |
| | | | | FPEPVTVSWNSGALTSGVHTFPAVLQSSGLY | ISSLQAEDVAVYYCQQ |
| | | | | SLSSVVTVPSSSLGTQTYICNVNHKPSNTKVD | YYSTPFTFGPGTKVEI |
| | | | | KKVEPKSCDKTHTCPPCPAPELLGGPSVFLFP | KRTVAAPSVFIFPPSD |
| | | | | PKPKDTLMISRTPEVTCVVVDVSHEDPEVKFN | EQLKSGTASVVCLLNN |
| | | | | WYVDGVEVHNAKTKPREEQYNSTYRVVSVLT | FYPREAKVQWKVDNAL |
| | | | | VLHQDWLNGKEYKCKVSNKALPAPIEKTISKA | QSGNSQESVTEQDSKD |
| | | | | KGQPREPQVYTLPPSREEMTKNQVSLTCLVK | STYSLSSTLTLSKADY |
| | | | | GFYPSDIAVEWESNGQPENNYKTTPPVLDSD | EKHKVYACEVTHQGLS |
| | | | | GSFFLYSKLTVDKSRWQQGNVFSCSVMHEAL | SPVTKSFNRGEC |
| | | | | HNHYTQKSLSLSPGK | |
| TIGIT | etigi- | 486 | 487 | QVQLQESGPGLVKPSETLSLTCAVSGYSITSD | DIQMTQSPSSLSASVG |
| | limab | | | YAWNWIRQPPGKGLEWIGYISYSGSTSYNPSL | DRVTITCKASQDVSTA |
| | | | | RSRVTISRDTSKNQFFLKLSSVTAADTAVYYC | VAWYQQKPGKAPKLLI |
| | | | | ARRQVGLGFAYWGQGTLVTVSSASTKGPSVF | YSASYRYTGVPSRFSG |
| | | | | PLAPSSKSTSGGTAALGCLVKDYFPEPVTVSW | SGSGTDFTFTISSLQP |
| | | | | NSGALTSGVHTFPAVLQSSGLYSLSSVVTVPS | EDIATYYCQQHYSTPW |
| | | | | SSLGTQTYICNVNHKPSNTKVDKRVEPKSCDK | TFGQGTKVEIKRTVAA |
| | | | | THTCPPCPAPELLGGPSVFLFPPKPKDTLMIS | PSVFIFPPSDEQLKSG |
| | | | | RTPEVTCVVVDVSHEDPEVKFNWYVDGVEVH | TASVVCLLNNFYPREA |
| | | | | NAKTKPREEQYNSTYRVVSVLTVLHQDWLNG | KVQWKVDNALQSGNSQ |
| | | | | KEYKCKVSNKALPAPIEKTISKAKGQPREPQV | ESVTEQDSKDSTYSLS |
| | | | | YTLPPSREEMTKNQVSLTCLVKGFYPSDIAVE | NTLTLSKADYEKHKVY |
| | | | | WESNGQPENNYKTTPPVLDSDGSFFLYSKLT | ACEVTHQGLSSPVTKS |
| | | | | VDKSRWQQGNVFSCSVMHEALHNHYTQKSL | FNRGEC |
| | | | | SLSPGK | |
| TIGIT | vibosto- | 488 | 489 | EVQLVQSGAEVKKPGSSVKVSCKASGYTFSS | DIQMTQSPSSLSASVG |
| | limab | | | YVMHWVRQAPGQGLEWIGYIDPYNDGAKYA | DRVTITCRASEHIYSY |
| | | | | QKFQGRVTLTSDKSTSTAYMELSSLRSEDTAV | LSWYQQKPGKVPKLLI |
| | | | | YYCARGGPYGWYFDVWGQGTTVTVSSASTK | YNAKTLAEGVPSRFSG |
| | | | | GPSVFPLAPSSKSTSGGTAALGCLVKDYFPEP | SGSGTDFTLTISSLQP |
| | | | | VTVSWNSGALTSGVHTFPAVLQSSGLYSLSSV | EDVATYYCQHHFGSPL |
| | | | | VTVPSSSLGTQTYICNVNHKPSNTKVDKKVEP | TFGQGTRLEIKRTVAA |
| | | | | KSCDKTHTCPPCPAPELLGGPSVFLFPPKPKD | PSVFIFPPSDEQLKSG |
| | | | | TLMISRTPEVTCVVVDVSHEDPEVKFNWYVD | TASVVCLLNNFYPREA |
| | | | | GVEVHNAKTKPREEQYNSTYRVVSVLTVLHQ | KVQWKVDNALQSGNSQ |
| | | | | DWLNGKEYKCKVSNKALPAPIEKTISKAKGQP | ESVTEQDSKDSTYSLS |
| | | | | REPQVYTLPPSRDELTKNQVSLTCLVKGFYPS | STLTLSKADYEKHKVY |
| | | | | DIAVEWESNGQPENNYKTTPPVLDSDGSFFLY | ACEVTHQGLSSPVTKS |
| | | | | SKLTVDKSRWQQGNVFSCSVMHEALHNHYT | FNRGEC |
| | | | | QKSLSLSPGK | |
| TIGIT | domvana- | 490 | 491 | EVQLVESGGGLVQPGGSLRLSCAASGFTFSN | DIQMTQSPSSLSASVG |
| | limab | | | FGMHWVRQAPGKGLEWVAFISSGSSSIYYAD | DRVTITCRASKSISKY |
| | | | | TVKGRFTISRDNAKNSLYLQMNSLRAEDTAVY | LAWYQQKPGKAPKLLI |
| | | | | YCARMRLDYYAMDYWGQGTMVTVSSASTKG | YSGSTLQSGVPSRFSG |
| | | | | PSVFPLAPSSKSTSGGTAALGCLVKDYFPEPV | SGSGTDFTLTISSLQP |
| | | | | TVSWNSGALTSGVHTFPAVLQSSGLYSLSSVV | EDFATYYCQQHNEYPW |
| | | | | TVPSSSLGTQTYICNVNHKPSNTKVDKKVEPK | TFGGGTKVEIKRTVAA |
| | | | | SCDKTHTCPPCPAPEAAGGPSVFLFPPKPKDT | PSVFIFPPSDEQLKSG |
| | | | | LMISRTPEVTCVVVDVSHEDPEVKFNWYVDG | TASVVCLLNNFYPREA |
| | | | | VEVHNAKTKPREEQYNSTYRVVSVLTVLHQD | KVQWKVDNALQSGNSQ |
| | | | | WLNGKEYKCKVSNKALPAPIEKTISKAKGQPR | ESVTEQDSKDSTYSLS |
| | | | | EPQVYTLPPSRDELTKNQVSLTCLVKGFYPSD | STLTLSKADYEKHKVY |
| | | | | IAVEWESNGQPENNYKTTPPVLDSDGSFFLYS | ACEVTHQGLSSPVTKS |
| | | | | KLTVDKSRWQQGNVFSCSVMHEALHNHYTQ | FNRGEC |
| | | | | KSLSLSPG | |
| TIGIT | ociper- | 492 | 493 | EVQLVESGGGLVQPGGSLRLSCAASGFTFSD | EIVMTQSPATLSVSPG |
| | limab | | | YYMYWVRQAPGKGLEWVAYITKGGGSTYYP | ERATLSCKASQDVGTS |
| | | | | DSVKGRFTISRDNAKNTLYLQMNSLRAEDTAV | VAWYQQKPGQAPRLLI |
| | | | | YYCARQTNYDFTMDYWGQGTLVTVSSASTKG | YWASARHTGIPARFSG |
| | | | | PSVFPLAPSSKSTSGGTAALGCLVKDYFPEPV | SGSGTEFTLTISSLQS |
| | | | | TVSWNSGALTSGVHTFPAVLQSSGLYSLSSVV | EDFAVYYCQQYSSYPL |
| | | | | TVPSSSLGTQTYICNVNHKPSNTKVDKKVEPK | TFGGGTKVEIKRTVAA |
| | | | | SCDKTHTCPPCPAPELLGGPSVFLFPPKPKDT | PSVFIFPPSDEQLKSG |
| | | | | LMISRTPEVTCVVVDVSHEDPEVKFNWYVDG | TASVVCLLNNFYPREA |
| | | | | VEVHNAKTKPREEQYNSTYRVVSVLTVLHQD | KVQWKVDNALQSGNSQ |
| | | | | WLNGKEYKCKVSNKALPAPIEKTISKAKGQPR | ESVTEQDSKDSTYSLS |
| | | | | EPQVYTLPPSREEMTKNQVSLTCLVKGFYPSD | STLTLSKADYEKHKVY |
| | | | | IAVEWESNGQPENNYKTTPPVLDSDGSFFLYS | ACEVTHQGLSSPVTKS |
| | | | | KLTVDKSRWQQGNVFSCSVMHEALHNHYTQ | FNRGEC |
| | | | | KSLSLSPGK | |
| TIGIT | belres- | 494 | 495 | QVQLVQSGAEVKKPGASVKVSCKASGYTFTS | EIVLTQSPGTLSLSPG |
| | totug | | | YYMHWVRQAPGQGLEWMGVIGPSGASTSYA | ERATLSCRASQSVRSS |
| | | | | QKFQGRVTLTRDTSTSTVYMELSSLRSEDTAV | YLAWYQQKPGQAPRLL |
| | | | | YYCARDHSDYWSGIMEVWGQGTTVTVSSAST | IYGASSRATGIPDRFS |
| | | | | KGPSVFPLAPSSKSTSGGTAALGCLVKDYFPE | GSGSGTDFTLTISRLE |
| | | | | PVTVSWNSGALTSGVHTFPAVLQSSGLYSLSS | PEDFAVYYCQQYFSPP |
| | | | | VVTVPSSSLGTQTYICNVNHKPSNTKVDKKVE | WTFGGGTKVEIKRTVA |
| | | | | PKSCDKTHTCPPCPAPELLGGPSVFLFPPKPK | APSVFIFPPSDEQLKS |
| | | | | DTLMISRTPEVTCVVVDVSHEDPEVKFNWYVD | GTASVVCLLNNFYPRE |
| | | | | GVEVHNAKTKPREEQYNSTYRVVSVLTVLHQ | AKVQWKVDNALQSGNS |
| | | | | DWLNGKEYKCKVSNKALPAPIEKTISKAKGQP | QESVTEQDSKDSTYSL |
| | | | | REPQVYTLPPSRDELTKNQVSLTCLVKGFYPS | SSTLTLSKADYEKHKV |
| | | | | DIAVEWESNGQPENNYKTTPPVLDSDGSFFLY | YACEVTHQGLSSPVTK |
| | | | | SKLTVDKSRWQQGNVFSCSVMHEALHNHYT | SFNRGEC |
| | | | | QKSLSLSPGK | |
| TIGIT | dargis- | 496 | 497 | QVQLVQSGSELKKPGASVKVSCKASGYTFTS | DIQLTQSPSFLSASVG |
| | totug | | | YPMNWVRQAPGQGLEWMGWINTNTGNPTYA | DRVTITCRASQGISSY |
| | | | | QGFTGRFVFSLDTSVSTAYLQISSLKAEDTAVY | LAWYQQKPGKAPKLLI |
| | | | | YCARVGGYSVDEYAFDVWGQGTLVTVSSAST | YAASTLQSGVPSRFSG |
| | | | | KGPSVFPLAPSSKSTSGGTAALGCLVKDYFPE | SGSGTEFTLTISSLQP |
| | | | | PVTVSWNSGALTSGVHTFPAVLQSSGLYSLSS | EDFATYYCQQLSSYPT |
| | | | | VVTVPSSSLGTQTYICNVNHKPSNTKVDKRVE | FGGGTKVEIKRTVAAP |
| | | | | PKSCDKTHTCPPCPAPELLGGPSVFLFPPKPK | SVFIFPPSDEQLKSGT |
| | | | | DTLMISRTPEVTCVVVDVSHEDPEVKFNWYVD | ASVVCLLNNFYPREAK |
| | | | | GVEVHNAKTKPREEQYNSTYRVVSVLTVLHQ | VQWKVDNALQSGNSQE |
| | | | | DWLNGKEYKCKVSNKALPAPIEKTISKAKGQP | SVTEQDSKDSTYSLSS |
| | | | | REPQVYTLPPSREEMTKNQVSLTCLVKGFYPS | TLTLSKADYEKHKVYA |
| | | | | DIAVEWESNGQPENNYKTTPPVLDSDGSFFLY | CEVTHQGLSSPVTKSF |
| | | | | SKLTVDKSRWQQGNVFSCSVMHEALHNHYT | NRGEC |
| | | | | QKSLSLSPG | |
| TIGIT | ralzapas- | 498 | 499 | QVQLQESGPGLVKPSGTLSLTCAVSGYSITSG | EIVMTQSPATLSVSPG |
| | totug | | | YSWHWVRQPPGKGLEWIGYVHYSGSTNYNP | ERATLSCKASQDVRTA |
| | | | | SLKSRVTISVDKSKNQFSLKLSSVTAADTAVYY | VAWYQQKPGQAPRLLI |
| | | | | CARMDYGNYGGAMDYWGQGTLVTVSSASTK | YSASYRYTGIPARFSG |
| | | | | GPSVFPLAPSSKSTSGGTAALGCLVKDYFPEP | SGSGTEFTLTISSLQS |
| | | | | VTVSWNSGALTSGVHTFPAVLQSSGLYSLSSV | EDFAVYYCQQYYSTQW |
| | | | | VTVPSSSLGTQTYICNVNHKPSNTKVDKKVEP | TFGGGTKVEIKRTVAA |
| | | | | KSCDKTHTCPPCPAPELLGGPSVFLFPPKPKD | PSVFIFPPSDEQLKSG |
| | | | | TLMISRTPEVTCVVVDVSHEDPEVKFNWYVD | TASVVCLLNNFYPREA |
| | | | | GVEVHNAKTKPREEQYNSTYRVVSVLTVLHQ | KVQWKVDNALQSGNSQ |
| | | | | DWLNGKEYKCKVSNKALPAPIEKTISKAKGQP | ESVTEQDSKDSTYSLS |
| | | | | REPQVYTLPPSRDELTKNQVSLTCLVKGFYPS | STLTLSKADYEKHKVY |
| | | | | DIAVEWESNGQPENNYKTTPPVLDSDGSFFLY | ACEVTHQGLSSPVTKS |
| | | | | SKLTVDKSRWQQGNVFSCSVMHEALHNHYT | FNRGEC |
| | | | | QKSLSLSPGK | |
| TIM-3 | cobolimab | 500 | 501 | EVQLLESGGGLVQPGGSLRLSCAAASGFTFS | DIQMTQSPSSLSASVG |
| | | | | SYDMSWVRQAPGKGLDWVSTISGGGTYTYY | DRVTITCRASQSIRRY |
| | | | | QDSVKGRFTISRDNSKNTLYLQMNSLRAEDTA | LNWYHQKPGKAPKLLI |
| | | | | VYYCASMDYWGQGTTVTVSSASTKGPSVFPL | YGASTLQSGVPSRFSG |
| | | | | APCSRSTSESTAALGCLVKDYFPEPVTVSWN | SGSGTDFTLTISSLQP |
| | | | | SGALTSGVHTFPAVLQSSGLYSLSSVVTVPSS | EDFAVYYCQQSHSAPL |
| | | | | SLGTKTYTCNVDHKPSNTKVDKRVESKYGPP | TFGGGTKVEIKRTVAA |
| | | | | CPPCPAPEFLGGPSVFLFPPKPKDTLMISRTP | PSVFIFPPSDEQLKSG |
| | | | | EVTCVVVDVSQEDPEVQFNWYVDGVEVHNA | TASVVCLLNNFYPREA |
| | | | | KTKPREEQFNSTYRVVSVLTVLHQDWLNGKE | KVQWKVDNALQSGNSQ |
| | | | | YKCKVSNKGLPSSIEKTISKAKGQPREPQVYTL | ESVTEQDSKDSTYSLS |
| | | | | PPSQEEMTKNQVSLTCLVKGFYPSDIAVEWES | STLTLSKADYEKHKVY |
| | | | | NGQPENNYKTTPPVLDSDGSFFLYSRLTVDKS | ACEVTHQGLSSPVTKS |
| | | | | RWQEGNVFSCSVMHEALHNHYTQKSLSLSLG | FNRGEC |
| | | | | K | |
| TIM-3 | sabato- | 502 | 503 | QVQLVQSGAEVKKPGSSVKVSCKASGYTFTS | AIQLTQSPSSLSASVG |
| | limab | | | YNMHWVRQAPGLGLEWMGDIYPGNGDTSYN | DRVTITCRASESVEYY |
| | | | | QKFKGRFTITADKSTSTVYMELSSLRSEDTAV | GTSLMQWYQQKPGKAP |
| | | | | YYCARVGGAFPMDYWGQGTTVTVSSASTKG | KLLIYAASNVESGVPS |
| | | | | PSVFPLAPCSRSTSESTAALGCLVKDYFPEPV | RFSGSGSGTDFTLTIS |
| | | | | TVSWNSGALTSGVHTFPAVLQSSGLYSLSSVV | SLQPEDFATYFCQQSR |
| | | | | TVPSSSLGTKTYTCNVDHKPSNTKVDKRVESK | KDPSTFGGGTKVEIKR |
| | | | | YGPPCPPCPAPEFLGGPSVFLFPPKPKDTLMI | TVAAPSVFIFPPSDEQ |
| | | | | SRTPEVTCVVVDVSQEDPEVQFNWYVDGVEV | LKSGTASVVCLLNNFY |
| | | | | HNAKTKPREEQFNSTYRVVSVLTVLHQDWLN | PREAKVQWKVDNALQS |
| | | | | GKEYKCKVSNKGLPSSIEKTISKAKGQPREPQ | GNSQESVTEQDSKDST |
| | | | | VYTLPPSQEEMTKNQVSLTCLVKGFYPSDIAV | YSLSSTLTLSKADYEK |
| | | | | EWESNGQPENNYKTTPPVLDSDGSFFLYSRL | HKVYACEVTHQGLSSP |
| | | | | TVDKSRWQEGNVFSCSVMHEALHNHYTQKSL | VTKSFNRGEC |
| | | | | SLSLG | |
| TIM-3 | surzebi- | 504 | 505 | EVQLLESGGGLVQPGGSLRLSCAAASGFTFS | EIVLTQSPATLSVSPGERATLSCRASESVEY |
| | climab | | | YAMSWVRQAPGKGLEWVAAISSGGSLYYPDS | YGTSLMQWYQQKPGQAPRLLIYAASNVESG |
| | | | | VKGRFTISRDNAKNTLYLQMNSLRAEDTAVYY | IPARFSGSGSGTEFTLTISSLQSEDFAVYYCQ |
| | | | | CARGREADGGYFDYWGQGTLVTVSSASTKG | QSLKVPLTFGGGTKVEIKRTVAAPSVFIFPPS |
| | | | | PSVFPLAPSSKSTSGGTAALGCLVKDYFPEPV | DEQLKSGTASVVCLLNNFYPREAKVQWKVD |
| | | | | TVSWNSGALTSGVHTFPAVLQSSGLYSLSSVV | NALQSGNSQESVTEQDSKDSTYSLSSTLTLS |
| | | | | TVPSSSLGTQTYICNVNHKPSNTKVDKKVEPK | KADYEKHKVYACEVTHQGLSSPVTKSFNRGE |
| | | | | SCDKTHTCPPCPAPPAAGPSVFLFPPKPKDTL | C |
| | | | | MISRTPEVTCVVVDVSHEDPEVKFNWYVDGV | |
| | | | | EVHNAKTKPREEQYNSTYRVVSVLTVLHQDW | |
| | | | | LNGKEYKCKVSNKALAAPIEKTISKAKGQPREP | |
| | | | | QVYTLPPSRDELTKNQVSLTCLVKGFYPSDIA | |
| | | | | VEWESNGQPENNYKTTPPVLDSDGSFFLYSK | |
| | | | | LTVDKSRWQQGNVFSCSVMHEALHNHYTQK | |
| | | | | SLSLSPGK | |

In some embodiments, the antibody or antigen-binding portion thereof may be derived from a mammalian species, for example, mice, rats, rabbits, a camelid (for example, llama), or human. Antibody variable regions can be those arising from one species, or they can be chimeric, containing segments of multiple species possibly further altered to optimize characteristics such as binding affinity or low immunogenicity. For human applications, it is desirable that the antibody has a human sequence. In the cases where the antibody or antigen-binding portion thereof is derived from a non-human species, the antibody or antigen-binding portion thereof may be humanized to reduce immunogenicity in a human subject. For example, if a human antibody of the desired specificity is not available, but such an antibody from a non-human species is, the non-human antibody can be humanized, e.g., through CDR grafting, in which the CDRs from the non-human antibody are placed into the respective positions in a framework of a compatible human antibody.

The following paragraphs provide non-exhaustive examples of known antibodies that bind to cell surface markers on immune cells (e.g., lymphocytes and monocytes). These antibodies or the antigen binding domains thereof can be used as binding moieties to target the disclosed tLNP. Collectively these antibodies and polypeptides comprising the antigen binding domains thereof constitute means for binding cell surface markers or means for binding immune cells.

In some embodiments, the tLNP is targeted to CD2+ cells and the binding moiety comprises the antigen binding domain of an anti-CD2 antibody. Accordingly, in some such embodiments, the antibody comprises OKT11, RPA-2.10, T111 (3T4-8B5), T112 (1OLD2-4C1), T113 (1Mono2A6), siplizumab, HuMCD2, TS2/18, TS1/8, AB75, LT-2, T6.3, MEM-65, OTI4E4, 9.1, 9.6, BTI-322, or an antigen-binding portion thereof. Each of these constitutes a means for binding CD2.

In some embodiments, the tLNP is targeted to CD3+ cells and the binding moiety comprises the antigen binding domain of an anti-CD3 antibody. In some embodiments, the surface antigen expressed by the target immune cell and recognized by the binding moiety of the tLNP is CD3 or a subunit thereof. CD3 is a T cell co-receptor involved in activating both the cytotoxic T cell (CD8+ T cells) and T helper cells (CD4+ T cells). In mammals, CD3 may comprise a CD3 gamma chain, a CD3 delta chain, two CD3 epsilon chains, and a homodimer of CD3 zeta chains. A TCR complex is formed by the association of CD3 with a TCR. Thus, a TCR complex may be composed of a CD3 gamma chain, a CD3 delta chain, two CD3 epsilon chains, a homodimer of CD3 zeta chains, a TCR alpha chain, and a TCR beta chain. Alternatively, a TCR complex may be composed of a CD3 gamma chain, a CD3 delta chain, two CD3 epsilon chains, a homodimer of CD3 zeta chains, a TCR gamma chain, and a TCR delta chain. In some embodiments, the CD3 subunit is CD3 epsilon chain. Accordingly, in some embodiments, the binding moiety is an antibody or an antigen-binding portion thereof specific to CD3 or a subunit thereof. Non-limiting examples of anti-CD3 antibodies or antigen-binding portions thereof include muromonab-CD3 (OKT3, non-humanized parental antibody of teplizumab; targets CD3 epsilon chain), alnuctamab (targets CD3 epsilon chain), teplizumab (TZIELD™, PRV-031, or MGA031; targets CD3 epsilon chain), otelixizumab (TRX4; targets epsilon chain), visilizumab (Nuvion®; targets CD3 epsilon chain), cevostamab (BFCR4350A; a CD3/FcRH5 bispecific antibody), teclistamab (TECVAYLI™; a CD3/B-cell maturation antigen (BCMA) bispecific antibody), elranatamab (PF-06863135; a CD3/BCMA bispecific antibody), pavurutamab (AMG 701; a CD3/BCMA bispecific antibody), vibecotamab (XmAb® 14045; a CD3/CD123 bispecific antibody), odronextamab (REGN1979; a CD3/CD20 bispecific antibody), foralumab (TZLS-401; targets CD3 epsilon chain), TR66 (targets CD3 epsilon chain), UCHT1v9, SP34, L2K, and any combinations or fragments thereof. Further anti-CD3 antibodies include acapatamab, alnuctamab, blinatumomab, and others listed in Table 2. In certain of these embodiments, the anti-CD3 antibodies or antigen-binding portions thereof may constitute means for binding to CD3 expressed on the surface of the immune cell, means for altering CD3 signal transduction, means for promotes transcription and/or translation of the internal payload, and/or means for conditioning the immune cell.

In some embodiments, the tLNP is targeted to CD4+ cells and the binding moiety comprises the antigen binding domain of an anti-CD4 antibody. Accordingly, in some such embodiments, the antibody comprises ibalizumab, inezetamab, semzuvolimab, zanolimumab, tregalizumab, UB-421, priliximab, MTRX1011A, cedelizumab, clenoliximab, keliximab, M-T413, TRX1, hB-F5, MAX.16H5, IT208, or an antigen-binding portion thereof. Each of these constitutes a means for binding CD4.

In some embodiments, the tLNP is targeted to CD5+ cells and the binding moiety comprises the antigen binding domain of an anti-CD5 antibody. Accordingly, in some such embodiments, the antibody comprises 5D7, UCHT2, L17F12, H65, HE3, OKT1, CRIS-1, MAT304, as well as those disclosed in WO1989006968, WO2008121160, U.S. Pat. No. 8,679,500, WO2010022737, WO2019108863, WO2022040608, or WO2022127844, each of which is incorporated by reference for all that they teach about anti-CD5 antibodies and their properties, or an antigen-binding portion thereof. Each of these constitutes a means for binding CD5.

In some embodiments, the tLNP is targeted to CD7+ cells and the binding moiety comprises the antigen binding domain of an anti-CD7 antibody. Accordingly, in some such embodiments, the antibody comprises TH-69, 3A1E, 3A1F, Huly-m2, WT1, YTH3.2.6, T3-3A1, grisnilimab, VHH-6, as well as those disclosed in U.S. Pat. No. 10,106,609, WO2017213979, WO2018098306, U.S. Pat. No. 11,447,548, WO2022136888, WO2020212710, WO2021160267, WO2022095802, WO2022095803, WO2022151851, or WO2022257835 each of which is incorporated by reference for all that they teach about anti-CD7 antibodies and their properties, or an antigen-binding portion thereof. Each of these constitutes a means for binding CD7.

In some embodiments, the tLNP is targeted to CD8+ cells and the binding moiety comprises the antigen binding domain of an anti-CD8 antibody. Accordingly, in some such embodiments, the antibody comprises crefmirlimab, 3B5, SP-16, LT8, 17D8, MEM-31, MEM-87, RIV11, UCHT4, YTC182.20, RPA-T8, OKT8, SK1, YTC182.20, 51.1, TRX2, MT807, IAB22M, HIT8a, C8/144B, RAVB3, SIDI8BEE, BU88, EPR26538-16, 2ST8.5H7, as well as those disclosed in U.S. Pat. No. 10,414,820, WO2015184203, WO2017134306, WO2019032661, WO2020060924, U.S. Pat. No. 10,730,944, WO2019033043, WO2021046159, WO2021127088, WO2022081516, U.S. Pat. No. 11,535,869, or WO2023004304 each of which is incorporated by reference for all that they teach about anti-CD8 antibodies and their properties, or an antigen-binding portion thereof. Each of these constitutes a means for binding CD8.

In some embodiments, the tLNP is targeted to CD10+ cells and the binding moiety comprises the antigen binding domain of an anti-CD10 antibody. Accordingly, in some such embodiments, the antibody comprises the one produced by the hybridoma represented by Accession No. NITE BP-02489 (disclosed in WO2018235247 which is incorporated by reference for all that they teach about anti-CD10 antibodies and their properties), FR4D11, or REA877, or an antigen-binding portion thereof. Each of these constitutes a means for binding CD10.

In some embodiments, the tLNP is targeted to CD11a+ cells and the binding moiety comprises the antigen binding domain of an anti-CD11a antibody. Accordingly, in some such embodiments, the antibody comprises odulimomab, efalizumab, MAB107, or A122pAcF. Each of these antibodies constitutes a means for binding CD11a.

In some embodiments, the tLNP is targeted to CD11 b+ cells and the binding moiety comprises the antigen binding domain of an anti-CD11 b antibody. Accordingly, in some such embodiments, the antibody comprises ASD141 or MAB107 as well as those disclosed in US20150337039, U.S. Pat. No. 10,738,121, WO2016197974, U.S. Pat. No. 10,919,967, or WO2022147338 each of which is incorporated by reference for all that they teach about anti-CD11 b antibodies and their properties, or an antigen-binding portion thereof. Each of these constitutes a means for binding CD11 b.

In some embodiments, the tLNP is targeted to CD13+ cells and the binding moiety comprises the antigen binding domain of an anti-CD13 antibody. CD13 is also known as aminopeptidase N (APN). Accordingly, in some such embodiments, the antibody comprises MT95-4 or Nbl57 (disclosed in WO2021072312 which is incorporated by reference for all that they teach about anti-CD13 antibodies and their properties), as well as those disclosed in WO2023037015 which is incorporated by reference for all that it teaches about anti-CD13 antibodies and their properties, or an antigen-binding portion thereof. Each of these constitutes a means for binding CD13.

In some embodiments, the tLNP is targeted to CD14+ cells and the binding moiety comprises the antigen binding domain of an anti-CD14 antibody. Accordingly, in some such embodiments, the antibody comprises atibuclimab or r18D11 as well as those disclosed in WO2018191786 or WO2015140591 each of which is incorporated by reference for all that they teach about anti-CD14 antibodies and their properties, or an antigen-binding portion thereof. Each of these constitutes a means for binding CD14.

In some embodiments, the tLNP is targeted to CD16a+ cells and the binding moiety comprises the antigen binding domain of an anti-CD16a antibody. Accordingly, in some such embodiments, the antibody comprises AFM13, sdA1, sdA2, or hu3G8-5.1-N297Q as well as those disclosed in U.S. Pat. No. 11,535,672, WO2018158349, WO2007009065, U.S. Pat. No. 10,385,137, WO2017064221, U.S. Pat. No. 10,758,625, WO2018039626, WO2018152516, WO2021076564, WO2022161314, or WO2023274183 each of which is incorporated by reference for all that they teach about anti-CD16A antibodies and their properties, or an antigen-binding portion thereof. Each of these constitutes a means for binding CD16a.

In some embodiments, the tLNP is targeted to CD25+ cells and the binding moiety comprises the antigen binding domain of an anti-CD25 antibody. Accordingly, in some such embodiments, the antibody comprises daclizumab, basiliximab, camidanlumab, tesirine, inolimomab, RO7296682, HuMax-TAC, CYT-91000, STI-003, RTX-003, or an antigen-binding portion thereof. Each of these constitutes a means for binding CD25.

In some embodiments, the tLNP is targeted to CD28+ cells and the binding moiety comprises the antigen binding domain of an anti-CD28 antibody. Accordingly, in some such embodiments, the antibody comprises GN1412, acazicolcept, lulizumab, prezalumab, theralizumab, FR104CD, and davoceticept, as well as those disclosed in U.S. Pat. Nos. 8,454,959, 8,785,604, 11,548,947, 11,530,268, 11,453,721, 11,591,401, WO2002030459, WO2002047721, US20170335016, US20200181260, U.S. Pat. No. 11,608,376, WO2020127618, WO2021155071, or WO2022056199 each of which is incorporated by reference for all that they teach about anti-CD28 antibodies and their properties, or an antigen-binding portion thereof. Each of these constitutes a means for binding CD28.

In some embodiments, the tLNP is targeted to CD29+ cells and the binding moiety comprises the antigen binding domain of an anti-CD29 antibody. Accordingly, in some such embodiments, the antibody comprises OS2966, 6D276, 12G10, REA1060, as well as those disclosed in US20220372132 which is incorporated by reference for all that it teaches about anti-CD29 antibodies and their properties, or an antigen-binding portion thereof. Each of these constitutes a means for binding CD29.

In some embodiments, the tLNP is targeted to CD32A+ cells and the binding moiety comprises the antigen binding domain of an anti-CD32A antibody. Accordingly, in some such embodiments, the antibody comprises VIB9600, humanized IV.3, humanized AT-10, or MDE-8 as well as those disclosed in U.S. Pat. Nos. 9,688,755, 9,284,375, 9,382,321, 11,306,145, or WO2022067394 each of which is incorporated by reference for all that they teach about anti-CD32A antibodies and their properties, or an antigen-binding portion thereof. Each of these constitutes a means for binding CD32A.

In some embodiments, the tLNP is targeted to CD34+ cells and the binding moiety comprises the antigen binding domain of an anti-CD34 antibody. Accordingly, in some such embodiments, the antibody comprises h4C8, 9C5, 2E10, 5B12, REA1164, C5B12, C2e10, My10, OBend/10, as well as those disclosed in WO2009079922, WO2023141297, WO2015121383, U.S. Pat. No. 8,927,696, or 8,399,249, each of which is incorporated by reference for all that they teach about anti-CD34 antibodies and their properties, or an antigen-binding portion thereof. Each of these constitutes a means for binding CD34.

In some embodiments, the tLNP is targeted to CD40+ cells and the binding moiety comprises the antigen binding domain of an anti-CD40 antibody. Accordingly, in some such embodiments, the antibody comprises cifurtilimab, sotigalimab, iscalimab, dacetuzumab, selicrelumab, bleselumab, lucatumumab, giloralimab, ravagalimab, tecaginlimab, teneliximab, or mitazalimab as well as those disclosed in U.S. Pat. No. 10,633,444, each of which is incorporated by reference for all that they teach about anti-CD40 antibodies and their properties, or an antigen-binding portion thereof. Each of these constitutes a means for binding CD40.

In some embodiments, the tLNP is targeted to CD44+ cells and the binding moiety comprises the antigen binding domain of an anti-CD44 antibody. Accordingly, in some such embodiments, the antibody comprises RO5429083, VB6-008, PF-03475952, or RG7356, as well as those disclosed in WO2008144890, U.S. Pat. No. 8,383,117, WO2008079246, US20100040540, WO2015076425, U.S. Pat. No. 9,220,772, US20140308301, WO2020159754, WO2021160269, WO2021178896, WO2022022749, WO2022022720, or WO2022243838, each of which is incorporated by reference for all that they teach about anti-CD44 antibodies and their properties, or an antigen-binding portion thereof. Each of these constitutes a means for binding CD44.

In some embodiments, the tLNP is targeted to CD56+ cells and the binding moiety comprises the antigen binding domain of an anti-CD56 antibody. Accordingly, in some such embodiments, the antibody comprises lorvotuzumab, adcitmer, or promiximab, as well as those disclosed in WO2012138537, U.S. Pat. Nos. 10,548,987, 10,730,941, or US20230144142, each of which is incorporated by reference for all that they teach about anti-CD56 antibodies and their properties, or an antigen-binding portion thereof. Each of these constitutes a means for binding CD56.

In some embodiments, the tLNP is targeted to CD64+ cells and the binding moiety comprises the antigen binding domain of an anti-CD64 antibody. Accordingly, in some such embodiments, the antibody comprises HuMAb 611 or H22 as well as those disclosed in U.S. Pat. No. 7,378,504, WO2014083379, US20170166638, or WO2022155608 each of which is incorporated by reference for all that they teach about anti-CD64 antibodies and their properties, or an antigen-binding portion thereof. Each of these constitutes a means for binding CD64.

In some embodiments, the tLNP is targeted to CD68+ cells and the binding moiety comprises the antigen binding domain of an anti-CD68 antibody. Accordingly, in some such embodiments, the antibody comprises Ki-M7, PG-M1, 514H12, ABM53F5, 3F7C6, 3F7D3, Y1/82A, EPR20545, CDLA68-1, LAMP4-824, or an antigen-binding portion thereof. Each of these constitutes a means for binding CD68.

In some embodiments, the tLNP is targeted to CD70+ cells and the binding moiety comprises the antigen binding domain of an anti-CD70 antibody. Accordingly, in some such embodiments, the antibody comprises cusatuzumab, vorsetuzumab, MDX-1203, MDX-1411, AMG-172, SGN-CD70A, ARX305, PRO1160, as well as those disclosed in U.S. Pat. Nos. 9,765,148, 8,124,738, IS10,266,604, WO2021138264, U.S. Pat. Nos. 9,701,752, 10,108,123, WO2014158821, U.S. Pat. No. 10,689,456, WO2017062271, U.S. Pat. Nos. 11,046,775, 11,377,500, WO2021055437, WO2021245603, WO2022002019, WO2022078344, WO2022105914, WO2022143951, WO2023278520, WO2022226317, WO2022262101, U.S. Pat. No. 11,613,584, or WO2023072307, each of which is incorporated by reference for all that they teach about anti-CD70 antibodies and their properties, or an antigen-binding portion thereof. Each of these constitutes a means for binding CD70.

In some embodiments, the tLNP is targeted to CD73+ cells and the binding moiety comprises the antigen binding domain of an anti-CD73 antibody. Accordingly, in some such embodiments, the antibody comprises oleclumab, uliledlimab, mupadolimab, dalutrafusp, dresbuxelimab, AK119, IBI325, BMS-986179, NZV930, JAB-BX102, Sym024, TB19, TB38, HBM1007, 3F7, mAb19, Hu001-MMAE, IPH5301, or INCA00186, as well as those disclosed in U.S. Pat. Nos. 9,938,356, 10,584,169, WO2022083723, WO2022037531, WO2021213466, WO2022083049, U.S. Pat. No. 10,822,426, WO2021259199, U.S. Pat. Nos. 10,100,129, 11,312,783, 11,174,319, 11,634,500, WO2021138467, WO2017118613, U.S. Pat. No. 9,388,249, WO2020216697, U.S. Pat. Nos. 11,180,554, 11,530,273, WO2019173692, WO2019170131, U.S. Pat. No. 11,312,785, WO2020098599, WO2020143836, WO2020143710, U.S. Pat. Nos. 11,034,771, 11,299,550, WO2020253568, WO2021017892, WO2021032173, WO2021032173, WO2021097223, WO2021205383, WO2021227307, WO2021241729, WO2022096020, WO2022105881, WO2022179039, WO2022214677, or WO2022242758, each of which is incorporated by reference for all that they teach about anti-CD73 antibodies and their properties, or an antigen-binding portion thereof. Each of these constitutes a means for binding CD73.

In some embodiments, the tLNP is targeted to CD90+ cells and the binding moiety comprises the antigen binding domain of an anti-CD90 antibody. Accordingly, in some such embodiments, the antibody comprises REA897, OX7, 5E10, K117, L127, or an antigen-binding portion thereof. Each of these constitutes a means for binding CD90.

In some embodiments, the tLNP is targeted to CD105+ cells and the binding moiety comprises the antigen binding domain of an anti-CD105 antibody. Accordingly, in some such embodiments, the antibody comprises carotuximab, TRC205, or huRH105, as well as those disclosed in U.S. Pat. Nos. 8,221,753, 9,926,375, WO2010039873, WO2010032059, WO2012149412, WO2015118031, WO2021118955, US20220233591, or US20230075244, each of which is incorporated by reference for all that they teach about anti-CD105 antibodies and their properties, or an antigen-binding portion thereof. Each of these constitutes a means for binding CD105.

In some embodiments, the tLNP is targeted to CD117+ cells and the binding moiety comprises the antigen binding domain of an anti-CD117 antibody. Accordingly, in some such embodiments, the antibody comprises briquilimab, barzolvolimab, CDX-0158, LOP628, MGTA-117, NN2101, CK6, Ab85 (HIST1H2BC), 104D2, or SR1, as well as those disclosed in U.S. Pat. No. 7,915,391, WO2022159737, U.S. Pat. No. 9,540,443, WO2015050959, U.S. Pat. Nos. 9,789,203, 8,552,157, 10,406,179, 9,932,410, WO2019084067, WO2020219770, U.S. Pat. No. 10,611,838, WO2020076105, WO2021107566, U.S. Pat. No. 11,208,482, WO2021044008, WO2021099418, WO2022187050, or WO2023026791, WO2021188590, each of which is incorporated by reference for all that they teach about anti-CD117 antibodies and their properties, or an antigen-binding portion thereof. Each of these constitutes a means for binding CD117.

In some embodiments, the tLNP is targeted to CD133+ cells and the binding moiety comprises the antigen binding domain of an anti-CD133 antibody. Accordingly, in some such embodiments, the antibody comprises AC133, 293C3, CMab-43, or RW03, as well as those disclosed in WO2018045880, U.S. Pat. Nos. 8,722,858, 9,249,225, WO2014128185, U.S. Pat. Nos. 10,711,068, 10,106,623, WO2018072025, or WO2022022718, each of which is incorporated by reference for all that they teach about anti-CD133 antibodies and their properties, or an antigen-binding portion thereof. Each of these constitutes a means for binding CD133.

In some embodiments, the tLNP is targeted to CD137+ cells and the binding moiety comprises the antigen binding domain of an anti-CD137 antibody. CD137 is also known as 4-11BB. Accordingly, in some such embodiments, the antibody comprises acasunlimab, cinrebafusp, ensomafusp, tecaginlimab, YH004, urelumab (BMS-663513), utomilumab (PF-05082566), ADG106, LVGN6051, PRS-343, as well as those disclosed in WO2005035584, WO2012032433, WO2017123650, U.S. Pat. Nos. 11,203,643, 11,242,395, 11,555,077, US20230067770, U.S. Pat. Nos. 11,535,678, 11,440,966, WO2019092451, U.S. Pat. Nos. 10,174,122, 11,242,385, 10,716,851, WO2020011966, WO2020011964, or U.S. Pat. No. 11,447,558, each of which is incorporated by reference for all that they teach about anti-CD137 antibodies and their properties, or an antigen-binding portion thereof. Each of these constitutes a means for binding CD137.

In some embodiments, the tLNP is targeted to CD146+ cells and the binding moiety comprises the antigen binding domain of an anti-CD146 antibody. Accordingly, in some such embodiments, the antibody comprises imaprelimab, ABX-MA1, huAA98, M2H, or IM1-24-3, as well as those disclosed in U.S. Pat. Nos. 10,407,506, 10,414,825, 6,924,360, 9,447,190, WO2014000338, U.S. Pat. No. 9,782,500, WO2018220467, U.S. Pat. No. 11,427,648, WO2019133639, WO2019137309, WO2020132190, or WO2022082073, each of which is incorporated by reference for all that they teach about anti-CD146 antibodies and their properties, or an antigen-binding portion thereof. Each of these constitutes a means for binding CD146.

In some embodiments, the tLNP is targeted to CD166+ cells and the binding moiety comprises the antigen binding domain of an anti-CD166 antibody. Accordingly, in some such embodiments, the antibody comprises praluzatamab, AZN-L50, REA442, or AT002, as well as those disclosed in U.S. Pat. Nos. 10,745,481, 11,220,544, or WO2008117049, each of which is incorporated by reference for all that they teach about anti-CD166 antibodies and their properties, or an antigen-binding portion thereof. Each of these constitutes a means for binding CD166.

In some embodiments, the tLNP is targeted to CD200+ cells and the binding moiety comprises the antigen binding domain of an anti-CD200 antibody. Accordingly, in some such embodiments, the antibody comprises samalizumab, OX-104, REA1067, B7V3V2, HPAB-0260-YJ, or TTI-CD200, as well as those disclosed in WO2007084321 or WO2019126536, each of which is incorporated by reference for all that they teach about anti-CD200 antibodies and their properties, or an antigen-binding portion thereof. Each of these constitutes a means for binding CD200.

In some embodiments, the tLNP is targeted to CD205+ cells and the binding moiety comprises the antigen binding domain of an anti-CD205 antibody. CD205 is also known as DEC205. Accordingly, in some such embodiments, the antibody comprises 3G9-2D2 (a component of CDX-1401) or LY75_A1 (a component of MEN1309) as well as those disclosed in U.S. Pat. No. 8,236,318, 10,081,682, or 11,365,258, each of which is incorporated by reference for all that they teach about anti-CD205 antibodies and their properties, or an antigen-binding portion thereof. Each of these constitutes a means for binding CD205.

In some embodiments, the tLNP is targeted to CD271+ cells and the binding moiety comprises the antigen binding domain of an anti-CD271 antibody. Accordingly, in some such embodiments, the antibody comprises REA844 or REAL709 as well as those disclosed in WO2022166802 which is incorporated by reference for all that it teaches about anti-CD271 antibodies and their properties, or an antigen-binding portion thereof. Each of these constitutes a means for binding CD271.

In some embodiments, the tLNP is targeted to BMPR2+ cells and the binding moiety comprises the antigen binding domain of an anti-BMPR2 antibody. Accordingly, in some such embodiments, the antibody comprises TAB-071CL (Creative Biolabs) as well as those disclosed in U.S. Pat. No. 11,292,846 or WO2021174198, each of which is incorporated by reference for all that they teach about anti-BMPR2 antibodies and their properties, or an antigen-binding portion thereof. Each of these constitutes a means for binding BMPR2.

In some embodiments, the tLNP is targeted to CTLA-4+ cells and the binding moiety comprises the antigen binding domain of an anti-CTLA-4 antibody. Accordingly, in some such embodiments, the antibody comprises botensilimab, ipilimumab, nurulimab, quavonlimab, tremelimumab, zalifrelimab, ADG116, ADG126, ADU-1604, AGEN1181, BCD-145, BMS-986218, BMS-986249, BT-007, CS1002, GIGA-564, HBM4003, IBI310, JK08, JMW-3B3, JS007, KD6001, KN044, ONC-392, REGN4659, TG6050, XTX101, YH001, or an antigen-binding portion thereof. Each of these constitutes a means for binding CTLA-4.

In some embodiments, the tLNP is targeted to GD2+ cells and the binding moiety comprises the antigen binding domain of an anti-GD2 antibody. Accordingly, in some such embodiments, the antibody comprises dinutuximab, ganglidiximab, lorukafusp, naxitamab, nivatrotamab EMD 273063, hu14.18k322A, MORAb-028, 3F8BiAb, BCD-245, KM666, ATL301, Ektomab, as well as those disclosed in U.S. Pat. Nos. 9,777,068, 9,315,585, WO2004055056, U.S. Pat. Nos. 9,617,349, 9,493,740, US20210002384, U.S. Pat. No. 8,507,657, WO2001023573, WO2012071216, WO2018010846, U.S. Pat. No. 8,951,524, WO2023280880, U.S. Pat. No. 9,856,324, WO2015132604, WO2017055385, WO2019059771, WO2020020194, or an antigen-binding portion thereof. Each of these constitutes a means for binding GD2.

In some embodiments, the tLNP is targeted to GITR+ cells and the binding moiety comprises the antigen binding domain of an anti-GITR antibody. Accordingly, in some such embodiments, the antibody comprises ragifilimab, efaprinermin, efgivanermin, TRX518, INCAGN01876, MK-4166, AMG 228, MEDI1873, BMS-986156, REGN6569, ASP1951, MK-1248, FRA154, GWN323, JNJ-64164711, ATOR-1144, or an antigen-binding portion thereof. Each of these constitutes a means for binding GITR.

In some embodiments, the tLNP is targeted to BTLA+ cells and the binding moiety comprises the antigen binding domain of an anti-BTLA antibody. Accordingly, in some such embodiments, the antibody comprises icatolimab, LY3361237, ANB032, HFB200603, as well as those disclosed in WO2020024897, U.S. Pat. Nos. 11,396,545, 8,563,694, 8,580,259, 11,253,590, 9,896,507, 11,421,030, 11,384,146, and 11,352,428, or an antigen-binding portion thereof. Each of these constitutes a means for binding BTLA.

In some embodiments, the tLNP is targeted to low affinity IL-2 receptor+ cells (CD122+) and the binding moiety comprises the antigen binding domain of an anti-IL-2 receptor antibody. Accordingly, in some such embodiments, the antibody comprises ANV419, MiK-Beta-1, as well as those disclosed in U.S. Pat. Nos. 9,028,830, 10,472,423, WO2022212848, WO2022221409, WO2022258673, or WO2023078113, each of which is incorporated by reference for all that they teach about anti-CD122 antibodies and their properties, or an antigen-binding portion thereof. Each of these constitutes a means for binding IL-2 receptor or CD122.

In some embodiments, the tLNP is targeted to CD25+(IL-2Rα+) cells and the binding moiety comprises the antigen binding domain of an anti-CD25 receptor antibody. Accordingly, in some such embodiments, the antibody comprises daclizumab, basiliximab, camidanlumab (HuMax-TAC), inolimomab, RO7296682, CYT-91000, xenopax, Sti-003, RA8, RTX-003, as well as those disclosed in WO2006108670, U.S. Pat. Nos. 7,438,907, 10,752,691, 8,314,213, US20150010539, WO2017062271, WO2020102591, WO2022104009, US20220251232, WO2020145209, US20220195055, US20220289855, US20230174670, WO2022040417, US20220143205, WO2023016455, US20230159646, WO2023067194 each of which is incorporated by reference for all that they teach about anti-CD25 antibodies and their properties, or an antigen-binding portion thereof. Each of these constitutes a means for binding CD25.

In some embodiments, the tLNP is targeted to CD132+ (cytokine receptor common γ-chain+) cells and the binding moiety comprises the antigen binding domain of an anti-CD132 receptor antibody. Accordingly, in some such embodiments, the antibody comprises REGN7257, as well as those disclosed in U.S. Pat. Nos. 11,629,195, 10,246,512, WO2022150788, WO2022212848, WO2023078113, or an antigen-binding portion thereof. Each of these constitutes a means for binding CD132.

In some embodiments, the tLNP is targeted to IL-7 receptor+ cells and the binding moiety comprises the antigen binding domain of an anti-IL-7 receptor antibody (anti-CD127). Accordingly, in some such embodiments, the antibody compriseslusvertikimab, bempikibart, PF-06342647, GSK2618960, OSE-127, as well as those disclosed in WO2021222227, WO2020254827, U.S. Pat. No. 11,008,395, 10,392,441, 9,447,182, 9,150,653, or 8,298,535, each of which is incorporated by reference for all that they teach about anti-CD127 antibodies and their properties, or an antigen-binding portion thereof. Each of these constitutes a means for binding the low affinity IL-2 receptor, CD127.

In some embodiments, the tLNP is targeted to IL-12 receptor+ cells and the binding moiety comprises the antigen binding domain of an anti-IL-12 receptor antibody. The receptor comprises β1 (CD212) and β2 chains. Accordingly, in some such embodiments, the antibody comprises CBYY-10413, REA333, as well as those disclosed in U.S. Pat. Nos. 8,715,657, 8,574,573, WO2022031929, US20220177567, each of which is incorporated by reference for all that they teach about anti-IL-12 receptor antibodies and their properties, or an antigen-binding portion thereof. Each of these constitutes a means for binding the IL-12 receptor.

In some embodiments, the tLNP is targeted to IL-15 receptor α+ cells and the binding moiety comprises the antigen binding domain of an anti-IL-15 receptor a antibody. Accordingly, in some such embodiments, the antibody comprises MAB1472-100, MAB5511, JM7A4, 5E3E1, JM7A4, 2639B, or an antigen-binding portion thereof. Each of these constitutes a means for binding the IL-15 receptor α.

In some embodiments, the tLNP is targeted to IL-18 receptor α+ cells and the binding moiety comprises the antigen binding domain of an anti-IL-18 receptor a antibody. Accordingly, in some such embodiments, the antibody comprises H44, as well as those disclosed in U.S. Pat. No. 8,003,103, 8,257,707, or 8,883,975, each of which is incorporated by reference for all that they teach about anti-IL-18 receptor a antibodies and their properties, or an antigen-binding portion thereof. Each of these constitutes a means for binding the IL-18 receptor α.

In some embodiments, the tLNP is targeted to IL-21 receptor+ cells and the binding moiety comprises the antigen binding domain of an anti-IL-21 receptor antibody. Accordingly, in some such embodiments, the antibody comprises PF-05230900, 1D1C2, 19F5, 18A5, REA233, as well as those disclosed in U.S. Pat. No. 8,790,643, WO2007114861, U.S. Pat. No. 7,495,085, WO2004083249, WO2009143523, or U.S. Pat. No. 9,309,318, each of which is incorporated by reference for all that they teach about anti-IL-21 receptor antibodies and their properties, or an antigen-binding portion thereof. Each of these constitutes a means for binding the IL-21 receptor (CD360).

In some embodiments, the tLNP is targeted to LAG-3+ cells and the binding moiety comprises the antigen binding domain of an anti-LAG-3 antibody. Accordingly, in some such embodiments, the antibody comprises relatlimab, tebotelimab, favezelimab, fianlimab, miptenalimab, HLX26, ieramilimab, GSK2831781, INCAGN2385, RO7247669, encelimab, FS118, SHR-1802, Sym022, IBI110, IBI323, bavunalimab, tuparstobart, EMB-02, ABL501, INCA32459, AK129, B1754111, MGD013, MK-4280, REGN3767, TSR-033, or an antigen-binding portion thereof. Each of these constitutes a means for binding LAG-3.

In some embodiments, the tLNP is targeted to TIGIT+ cells and the binding moiety comprises the antigen binding domain of an anti-TIGIT antibody. Accordingly, in some such embodiments, the antibody comprises tiragolumab, etigilimab, vibostolimab (MK-7684), domvanalimab, ociperlimab, belresotug, dargistotug, ralzapastotug, BMS986207, ASP8374, IBI939, IBI321, JS006, AZD2936, HLX301, CON902, SEA-TGT, AGEN1777, BAT6021, BAT6005, and EOS-448, each of which is incorporated by reference for all that they teach about anti-TIGIT antibodies and their properties, or an antigen-binding portion thereof. Each of these constitutes a means for binding TIGIT.

In some embodiments, the tLNP is targeted to ICOS+ and the binding moiety comprises the antigen binding domain of an anti-ICOS (anti-CD278) antibody. Accordingly, in some such embodiments, the antibody comprises alomfilimab, feladilimab, vopratelimab, izuralimab, MEDI-570, as well as those disclosed in U.S. Pat. Nos. 9,376,493, 9,695,247, 10,023,635, 9,193,789, 9,957,323, 10,793,632, US20080199466, U.S. Pat. Nos. 11,629,189, 10,898,556, US20220098305, each of which is incorporated by reference for all that they teach about anti-ICOS antibodies and their properties, or an antigen-binding portion thereof. Each of these constitutes a means for binding ICOS.

In some embodiments, the tLNP is targeted to siglec-15+ and the binding moiety comprises the antigen binding domain of an anti-siglec-15 antibody. Accordingly, in some such embodiments, the antibody comprises NC318, AB-25E9, A9E8, DS-1501, as well as those disclosed in U.S. Pat. Nos. 11,390,675, 9,493,562, 8,575,316, WO2013147212, U.S. Pat. No. 9,447,192, WO2021190622, WO20212514132, WO2022095934, WO2022179466, WO2022198040, WO2022223004, WO2022228183, WO2022237819, WO2023093816, each of which is incorporated by reference for all that they teach about anti-siglet-15 antibodies and their properties, or an antigen-binding portion thereof. Each of these constitutes a means for binding siglec-15.

In some embodiments, the tLNP is targeted to B7H3+ and the binding moiety comprises the antigen binding domain of an anti-B7H3 antibody. Accordingly, in some such embodiments, the antibody comprises enoblituzumab, omburtamab, obrindatamab, ifinatamab, mitzotamab, TRL4542, MGC018, DS-7300a, MHB088C, XmAb808, BAT8009, or an antigen-binding portion thereof. Each of these constitutes a means for binding B7H3.

In some embodiments, the tLNP is targeted to MSCA-1+ cells and the binding moiety comprises the antigen binding domain of an anti-MSCA-1 antibody. Accordingly, in some such embodiments, the antibody comprises REAL219, W8B2, X9C₃, or an antigen-binding portion thereof. Each of these constitutes a means for binding MSCA-1.

In some embodiments, the tLNP is targeted to OX40+ cells and the binding moiety comprises the antigen binding domain of an anti-OX40 antibody. Accordingly, in some such embodiments, the antibody comprises MEDI6469, ivuxolimab, rocatinlimab, GSK3174998, BMS-986178, vonlerizumab, INCAGN1949, tavolimab, BGB-A445, INBRX-106, BAT6026, telazorlimab, ATOR-1015, efizonerimod (MEDI 6383), revdofilimab, cudarolimab, FS120, HFB301001, EMB-09, HLX51, Hu222, ABM193, vonlerolizumab (MOXR0916), or an antigen-binding portion thereof. Each of these constitutes a means for binding OX40.

In some embodiments, the tLNP is targeted to PD-1+ cells and the binding moiety comprises the antigen binding domain of an anti-PD-1 antibody. Accordingly, in some such embodiments, the antibody comprises nivolumab, pembrolizumab, acrixolimab, balstilimab, budigalimab, camrelizumab, fidasimtamab, finotonlimab, iparomlimab, ivonescimab, izuralimab, latikafusp, reozalimab, rosnilimab, sudubrilimab, toripalimab, sintilimab, tislelizumab, cemiplimab, spartalizumab, serplulimab, cadonilimab, penpulimab, dostarlimab, zeluvalimab, zimberelimab, retifanlimab, pucotenlimab, pidilizumab, pidilizumab, balstilimab, ezabenlimab, AK112, geptanolimab, cetrelimab, prolgolimab, tebotelimab, sasanlimab, SG001, vudalimab, MEDI5752, rulonilimab, peresolimab, IBI318, budigalimab, MEDI0680, pimivalimab, QL1706, AMG 404, RO7121661, lorigerlimab, nofazinlimab, sindelizumab, or an antigen-binding portion thereof. Each of these constitutes a means for binding PD-1.

In some embodiments, the tLNP is targeted to PODXL+ cells and the binding moiety comprises the antigen binding domain of an anti-PODXL antibody. Accordingly, in some such embodiments, the antibody comprises MA11738, HPAB-3334LY, HPAB-M0612-YC, REA246, REA157, as well as those disclosed in U.S. Pat. No. 9,334,324 or 11,267,898 each of which is incorporated by reference for all that they teach about anti-PODXL antibodies and their properties, or an antigen-binding portion thereof. Each of these constitutes a means for binding PODXL.

In some embodiments, the tLNP is targeted to Sca-1+ cells and the binding moiety comprises the antigen binding domain of an anti-Sca-1 antibody. Accordingly, in some such embodiments, the antibody comprises CPP32 4-1-18, 2D4-C9-F1, AMM22070N, or an antigen-binding portion thereof. Each of these constitutes a means for binding SCA-1.

In some embodiments, the tLNP is targeted to SSEA-3+ cells and the binding moiety comprises the antigen binding domain of an anti-SSEA-3 antibody. Accordingly, in some such embodiments, the antibody comprises MC631, 2A9, 8A7, ND-742, 3H420, as well as those disclosed in U.S. Pat. No. 11,643,456 or WO2021138378, each of which is incorporated by reference for all that they teach about anti-SSEA-3 antibodies and their properties, or an antigen-binding portion thereof. Each of these constitutes a means for binding SSEA-3.

In some embodiments, the tLNP is targeted to SSEA-4+ cells and the binding moiety comprises the antigen binding domain of an anti-SSEA-4 antibody. Accordingly, in some such embodiments, the antibody comprises ch28/11, REA101, MC-813-70, ND-942-80, as well as those disclosed in U.S. Pat. Nos. 11,446,379, 10,273,295, 11,643,456, WO2019190952, or WO2021044039, each of which is incorporated by reference for all that they teach about anti-SSEA-4 antibodies and their properties, or an antigen-binding portion thereof. Each of these constitutes a means for binding SSEA-4.

In some embodiments, the tLNP is targeted to Stro-1+ cells and the binding moiety comprises the antigen binding domain of an anti-Stro-1 antibody. Accordingly, in some such embodiments, the antibody comprises STRO-1, TUSP-2, as well as those disclosed in US20130122022, which is incorporated by reference for all that it teaches about anti-Stro-1 antibodies and their properties, or an antigen-binding portion thereof. Each of these constitutes a means for binding Stro-1.

In some embodiments, the tLNP is targeted to Stro-4+ cells and the binding moiety comprises the antigen binding domain of an anti-Stro-4 antibody. Accordingly, in some such embodiments, the antibody comprises STRO-4, efungumab, 4C5, as well as those disclosed in U.S. Pat. No. 7,722,869, US20110280881, U.S. Pat. Nos. 9,115,192, 10,273,294, 10,457,726, WO2023091148, each of which is incorporated by reference for all that they teach about anti-Stro-4 antibodies and their properties, or an antigen-binding portion thereof. Each of these constitutes a means for binding Stro-4 (also known as heat shock protein-90).

In some embodiments, the tLNP is targeted to SUSD2+ cells and the binding moiety comprises the antigen binding domain of an anti-SUSD2 antibody. Accordingly, in some such embodiments, the antibody comprises REA795, CBXS-3571, CBXS-1650, CBXS-1989, CBXS-1671, CBXS-1990, CBXS-3676, 1279B, EPR8913(2), W5C5, or an antigen-binding portion thereof. Each of these constitutes a means for binding SUSD2.

In some embodiments, the tLNP is targeted to TIM-3+ cells and the binding moiety comprises the antigen binding domain of an anti-TIM-3 antibody. Accordingly, in some such embodiments, the antibody comprises TQB2618, sabatolimab, cobolimab, RO7121661, INCAGN02390, AZD7789, surzebiclimab, LY3321367, Sym023, BMS-986258, SHR-1702, LY3415244, LB1410, or an antigen-binding portion thereof. Each of these constitutes a means for binding TIM-3.

In some embodiments, the tLNP is targeted to TREM2+ cells and the binding moiety comprises the antigen binding domain of an anti-TREM2 antibody. Accordingly, in some such embodiments, the antibody comprises PI37012 as well as those disclosed in U.S. Pat. Nos. 10,508,148, 10,676,525, WO2017058866, U.S. Pat. Nos. 11,186,636, 11,124,567, WO2020055975, U.S. Pat. No. 11,492,402, WO2020121195, WO2023012802, WO2021101823, WO2023047100, WO2022032293, WO2022241082, WO2023039450, or WO2023039612, each of which is incorporated by reference for all that they teach about anti-TREM2 antibodies and their properties, or an antigen-binding portion thereof. Each of these constitutes a means for binding TREM2.

The conditioning agent is to be delivered to 1) tumor cells (cancerous cells or stromal cells) for cancer treatments, 2) B lineage cells in the treatment of antibody-mediated autoimmunity, 3) CD4⁺ T cells, including Th17 cells, in the treatment of T cell-mediated autoimmunity, and 4) fibrotic or fibrogenic cells for the treatment of fibrosis. Accordingly, a tLNP will need a binding moiety expressed on the surface of the desired cell type.

Binding moieties arise in multiple contexts in the disclosed aspects and embodiments. Binding moieties are used to target nanoparticles which can be used to provide the conditioning agent, the reprogramming agent or both. Any receptor-ligand pair where one of the partners is expressed at the surface of the targeted cell can be used but most commonly an antibody, or an antigen binding portion thereof, is used. Antibody antigen binding domains can also be adapted to serve as the antigen binding domain of a reprogramming agent such as a CAR or immune cell engager, although many of the herein disclosed embodiments are indifferent to CAR specificity and thus relate to a generic CAR without a stated antigen specificity. Similarly, antibody antigen binding domains can be incorporated into BiTEs or other immune cell engagers, and as with the CAR the disclosed embodiments are generally indifferent to specificity and thus relate to a generic immune cell engager without a stated antigen specificity. Nonetheless, many of the antibodies discussed herein for targeting tumor cells, B cells, T cells, NK cells, monocytes, macrophages, and so on, are also suitable for providing the antigen binding portion of a CAR or immune cell engager. Throughout this disclosure particular antibodies and antibody specificities are disclosed in relation to one or more of these uses. That a particular antibody or antibody specificity is not mentioned explicitly in connection with one or another of these uses does not mean that there is not an embodiment in which it could be applied to that use. For example, an antibody or antibody specificity disclosed as useful in the targeting moiety of a tLNP to deliver a nucleic acid encoded conditioning agent can also be useful in the targeting moiety of a tLNP to deliver an engineering agent when the targeted antigen is expressed on an immune cell it would be advantageous to reprogram. Similarly, an antibody or antibody specificity disclosed as useful in the targeting moiety of a tLNP to deliver a nucleic acid encoded conditioning agent can also be useful to provide specificity to a reprogramming agent such as a CAR or immune cell engager when the targeted antigen is expressed on a tumor, autoimmunity-mediating, or other pathogenic cell. With the guidance of the present disclosure the skilled artisan will recognize such further applications of the disclosed antibodies and antibody specificities.

Targeting T cells by using binding moieties recognizing CD4, CD5, and other T cell markers (surface antigens) is discussed in WO2022/081702, WO2022/081694, WO2022/081699, each of which is incorporated by reference herein for all that they teach about tLNP and their use for targeting T cells in vivo that is not inconsistent with the present disclosure.

Binding moieties for targeting CD8 can be based, for example, on the anti-CD8 antibodies SP-16, 3B5, SP-16, LT8, 17D8, MEM-31, MEM-87, RIV11, UCHT4, or YTC182.20, the anti-CD8a antibodies OKT8, SK1, RPA-T8, IAB22M (a set of humanized antibody fragments derived from OKT8), (MT-807R1), TRX2, HIT8a, C8/144B, or RAVB3, or the anti-CD8@antibodies SIDI8BEE, BU88, EPR26538-16, or 2ST8.5H7. U.S. Pat. No. 10,414,820, which discloses minibodies and diabodies based on humanized OKT8 including minibodies IAb_M1b_CD8 and IAb_M2b_CD8, and the diabody IAb_CysDb3b_CD8, is incorporated by reference for all that it teaches about humanized anti-CD8 antibodies that is not inconsistent with the present disclosure.

Binding moieties for targeting CD2 can be based, for example, on the anti-CD2 antibodies alefacept, siplizumab (also known as MEDI-507, a humanized form of LO-CD2a), OKT11, TS2/18, TS1/8, AB75, RPA-2.10, LT-2, T6.3MEM-65, BTI-322, HuMCD2, T11.2, or OT14E4.

CD2 and CD8 binding moieties can also be used for targeting NK cells. To target NK cells but not T cells, binding moieties for the CD56 or CD16 surface antigens can be used. Anti-CD56 antibodies include lorvotuzumab and promiximab, both humanized antibodies. Anti-CD16 monoclonal antibodies include 3G8 (mouse), and A9, the human anti-CD16A binding moiety of AFM13 (see Reusch et al., mAbs 6(3): 727-738, 2014 and Wu et al., J Hematology & Oncology 8:Article 96, 2015).

There are also several approaches to site-specific conjugation. Particularly but not exclusively suitable for truncated forms of antibody, C-terminal extensions of native or artificial sequences containing a particularly accessible cysteine residue are commonly used. Partial reduction of cystine bonds in an antibody, for example, with tris(2-carboxy)phosphine (TCEP), can also generate thiol groups for conjugation which can be site-specific under defined conditions with an amenable antibody fragment. Alternatively, the C-terminal extension can contain a sortase A substrate sequence, LPXTG (SEQ ID NO: 14) which can then be functionalized in a reaction catalyzed by sortase A and conjugated to the PEG-lipid, including through click chemistry reactions (see, for example, Moliner-Morro et al., Biomolecules 10(12):1661, 2020 which is incorporated by reference herein for all that it teaches about antibody conjugations mediated by the sortase A reaction and/or click chemistry). For whole antibody and other forms comprising an Fc region, site-specific conjugation to either (or both) of two specific lysine residues (Lys248 and Lys288) can be accomplished without any change to or extension of the native antibody sequence by use of one of the AJICAP® reagents (see, for example, Matsuda et al., Molecular Pharmaceutics 18:4058-4066, 2021 and Fujii et al., Bioconjugate Chemistry https://doi.org/10.1021/acs.bioconjchem.3.c00040, 2023, which are incorporated by reference herein for all that they teach about conjugation of antibodies with AJICAP reagents). The AJICAP reagents are modified affinity peptides that bind to specific loci on the Fc and react with an adjacent lysine residue. The peptide is then cleaved with base to leave behind a thiol-functionalized lysine residue which can then undergo conjugation through maleimide or haloamide reactions, for example). Functionalization with azide or dibenzocyclooctyne (DBCO) for conjugation by click chemistry is also possible.

Accordingly, in some embodiments the binding moiety is conjugated to the PEG moiety of the PEG-lipid through a thiol modified lysine residue. In some embodiments the conjugation is through a cysteine residue in a native or added antibody sequence. In other embodiments, the conjugation is through a sortase A substrate sequence. In still other embodiments, the conjugation is through a specific lysine residue (Lys248 or Lys288) in the Fc region.

A number of monoclonal antibodies (mAb) that recognize tumor surface antigens are used in the art for cancer treatment either as investigational agents or as approved products. The antigens recognized by these mAb are good targets for the nanoparticle targeting moiety. In addition to providing a targeting domain for a targeted nanoparticle, these mAbs have binding domains that can be utilized in a CAR or as an anti-tumor specificity in a BiTE or other immune cell engager. Some of these antigens are also useful targets in the treatment of antibody-mediated or T cell-mediated autoimmunity or fibrosis. It should be noted that the level of tumor specificity sought for an anti-tumor treatment is not necessarily required for a tLNP delivering an encoded conditioning agent or soluble reprogramming agent (such as an immune cell engager) for expression in a tumor cell as expression of these agents in non-tumor cells would lead to those cells being factories for the agent but generally would not direct immune attack against these non-tumor cells. Nonetheless, in some embodiments, the targeting moiety for a tLNP delivering an encoded conditioning agent binds to the same antigen on the tumor, autoimmunity-mediating, or other pathogenic cell (and may be derived from the same antibody) as bound by an immune cell reprogramming agent (such as a CAR, TCR, or immune cell engager). In other embodiments, the targeting moiety for a tLNP delivering an encoded conditioning agent binds to a different antigen.

The mAbs themselves can be adapted to serve as the binding/targeting moiety itself, either as whole antibody, single chain Fv (scFv), F(ab), minibody, diabody, nanobody and the like, as can other antibodies with the same specificity. If the mAb is not human or humanized it will be preferred to humanize the antibody for use as the binding/targeting moiety so as to avoid inducing a human anti-animal antibody response and thereby facilitate repeat dosing. Some of the antibodies below have more than one specificity, so only the portion of the antibody having the indicated reactivity would be useful for targeting nanoparticles as described herein. The listing below does not necessarily identify every cancer or other condition in which the antigen can sometimes be found nor are the antigens necessarily found universally on the indicated cancer types. Accordingly, pairing of a targeting moiety and a cancer type will generally need to be confirmed for individual patients and cannot be made on the basis of cancer type alone. Some of the antibodies below have not been successful in clinical trials as cancer therapy, but this does not necessarily detract from their usefulness as a binding/target moiety. Most of the antigens listed below are expressed by the cancerous cells of the tumor, however a few, such as fibroblast activation protein (FAP), are expressed by stromal cells of the tumor. With respect to cancer treatment, expression of the conditioning agent in the stromal cells can still promote infiltration of immune cells into the tumor and will also alter the generally immunosuppressive tumor microenvironment to be more conducive to a productive immune response whether based on the engineering agent, a suppressed pre-existing antitumor response, a newly generated response to antigens of the cancerous cells, or some combination thereof. FAP is also a useful target in the treatment of fibrosis. With respect to the treatment of antibody-meditated autoimmunity, B cell lineage antigens are useful targets. With respect to T cell mediated autoimmunity CD4 and Th17 lineage antigens are useful targets. The antigens include:
Activin receptor-like kinase found in colorectal, liver, urogenital cancers and other solid tumors and bound by ascrinvacumab.
Adenocarcinoma antigen found on adenocarcinomas and bound by pintumomab.
α-fetoprotein found on liver cancer and bound by tacatuzumab.
AXL receptor tyrosine kinase found in multiple types of solid tumors: ovarian, cervical, endometrial, thyroid, non-small cell lung cancer, melanoma and sarcoma, and bound by enapotamab.
B cell maturation antigen (BCMA) found in multiple myeloma and bound by belantamab, elranatamab, and teclistamab. This B cell lineage antigen is also a useful target in the treatment of antibody-meditated autoimmunity.
CA-125 found on ovarian cancer and bound by igovomab, oregovomab, and sofituzumab.
CanAg (a glycoform of MUC1) found on colorectal and other cancers and bound by cantuzumab.
Carbonic anhydrase 9 found in clear cell renal carcinoma and bound by girentuximab.
Carcinoembryonic antigen (CEA) found on colorectal and gastrointestinal cancers and bound by altumomab and arcitumomab.
Carcinoembryonic antigen-related cell adhesion molecule 5 (CEACAM5) found in colorectal cancer and bound by tusamitamab, labetuzumab, and cibisatamab.
C—C chemokine receptor 4 (CCR4) found on adult T cell leukemia/lymphoma and bound by mogamulizumab.
C—C chemokine receptor 5 (CCR5) found on various solid tumors such as melanoma, pancreatic, breast (including triple negative breast cancer), prostate, colon, lung, liver, and stomach cancers and bound by Leronlimab.
CD4 found on T cells, including those mediating T cell-mediated autoimmunity and bound by tregalizumab, IT1208, UB-421 (humanized), and zanolimumab (human).
CD5 is a pan-T cell marker, the expression of which is often maintained in T cell cancers. It is bound by 5D7, HE3, telimomab and zolimomab.
CD19 found in acute lymphoblastic leukemia (ALL), large B cell lymphoma, diffuse large B cell lymphoma (DLBCL), and B cell non-Hodgkin lymphoma and bound by blinatumomab, coltuximab, denintuzumab, duvortuxizumab, inebilizumab, loncastuximab, tafasitamab, taplitumomab, and XMAB-5574. This B cell lineage antigen is also a useful target in the treatment of antibody-meditated autoimmunity.
CD20 found on B cell lymphoid cancers and bound by ibritumomab, obinutuzumab, ocaratuzumab, ocrelizumab, ofatumumab, rituximab, tositumumab, ublituximab, veltuzumab, mosentuzumab, FBTA05, epcoritamab, glofitamab, and odronextamab. This B cell lineage antigen is also a useful target in the treatment of antibody-meditated autoimmunity.
CD22 found in non-Hodgkin's lymphoma, hairy cell leukemia, and acute lymphoblastic leukemia and bound by bectumomab, epratuzumab, inotuzumab, moxetumomab, and pinatuzumab. This B cell lineage antigen is also a useful target in the treatment of antibody-meditated autoimmunity.
CD23 found in chronic lymphocytic leukemia and bound by lumiliximab and gomiliximab.
CD25 found in B-cell Hodgkin's lymphoma, non-Hodgkin lymphoma, acute lymphoblastic leukemia, and acute myeloid leukemia and bound by basiliximab, camidanlumab, daclizumab, and inolimomab.
CD28 found in chronic lymphocytic leukemia and bound by TGN1412 and lulizumab.
CD30 found in Hodgkin's lymphoma and bound by brentuximab.
CD33 found in acute myeloid leukemia and other myeloproliferative diseases and bound by lintuzumab, vadastuximab, and gemtuzumab.
CD37 found in B cell malignancies including Hodgkin's and non-Hodgkin's lymphoma and bound by lilotomab, naratuximab, otlertuzumab and tetulomab. This B cell lineage antigen is also a useful target in the treatment of antibody-meditated autoimmunity.
CD38 found in multiple myeloma and bound by daratumumab and isatuximab.
CD40 found on hematalogic cancers and bound by decetuzumab, bleselumab, iscalimab, lucatumumab, ravagalimab, selicrelumab, teneliximab, and vanalimab, and CD40L bound by toralizumab.
CD44 found in squamous cell carcinoma and bound by bivatuzumab.
CD51 found in metastatic prostate cancer and other solid tumors (including melanoma) and bound by abituzumab and intetumumab.
CD52 (CAMPATH-1) found on lymphatic cancers and bound by ALLO-647, gatralimab, and alemtuzumab.
CD56 found on small-cell lung and ovarian cancers, and Merkel cell carcinoma, and bound by lorvotuzumab.
CD70 found in renal cell carcinoma, non-Hodgkin's lymphoma, acute myeloid leukemia (AML) and myelodysplastic syndrome (MDS) and bound by cusatuzumab and vorsetuzumab.
CD73 (5′-nucleotidase) found on pancreatic, colorectal, and other cancers and bound by oleclumab, dresbuxelimab, and dalutrafusp.
CD74 found on multiple myeloma and other hematological malignancies and bound by milatuzumab.
CD79B found in B cell malignancies (such a non-Hodgkin's lymphoma) and bound by iladatuzumab and polatuzumab. This B cell lineage antigen is also a useful target in the treatment of antibody-meditated autoimmunity.
CD80 found in B cell lymphoma and bound by galiximab.
CD123 (IL-3Rα) found in leukemia including myeloid malignancies and bound by flotetuzumab, vibecotamab, and talacotuzumab.
CD159 found in gynecologic malignancies and other cancers and bound by monalizumab.
CD248 (endosialin) found on tumor stroma including in sarcomas and bound by ontuxizumab.
CD276 (B7-H3) found in head and neck cancer, melanoma, squamous cell cancer of the head and neck (SCCHN) and non-small cell lung cancer (NSCLC) and bound by enoblituzumab and omburtamab.
CD319 (SLAMF7) found in various hematologic cancers including multiple myeloma and bound by elotuzumab and azintuxizumab.
Claudin-18 isoform 2 is found on gastric tumors and bound by osemitamab zolbetuximab.
CLL1 is found on acute myeloid leukemia (AML) cells and leukemic stem cells and is bound by 27H4 (human), MCLL0517A (humanized) and CLT030 (an antibody drug conjugate using a humanized anti-CLL1 mAb).
C-type lectin domain family 12 member A (CLEC12A) found on myeloid blasts, atypical progenitor cells and leukemic stem cells and bound by epoditamab.
C—X—C chemokine receptor type 4 (CXCR-4) found on various types of cancer including breast cancer, ovarian cancer, melanoma, and prostate cancer, and bound by ulocuplumab.
Delta-like 3 (DLL3) found on small cell lung cancer and bound by rovalpituzumab.
Delta-like 4 (DLL4) found on pancreatic and non-small cell lung cancers and bound by demcizumab, enoticumab, and navicixizumab.
Epidermal Growth Factor like domain 7 (Egfl7) is found in colorectal cancer, hepatocellular carcinoma, and glioma and bound by parsatuzumab.
Endoglin found on angiosarcoma and bound by carotuximab.
EpCAM found in malignant ascites, colorectal, bladder, prostate, gastric, lung, breast, and ovarian cancers and bound by adecatumumab, catumaxomab, citatuzumab, edrecolomab, oportuzumab, solitomab, and tucotuzumab.
Eph receptor A3 (EPHA3) found on melanoma, breast, prostate, pancreatic, gastric, esophageal, and colon cancer, as well as hematopoietic tumors and bound by ifabotuzumab.
Epidermal growth factor receptor (EGFR) found in squamous cell carcinoma, head and neck cancer, glioma, glioblastoma, nasopharyngeal, colorectal, stomach, and non-small cell lung cancer and is bound by amivantamab, cetuximab, depatuxizumab, futuximab, imgatuzumab, laprituximab, losatuxizumab, matuzumab, modotuximab, necitumumab, nimotuzumab, panitumumab, tomuzotuximab, and zalutumumab.
Fibroblast activation protein (FAP) found on fibroblasts in tumor stroma and bound by sibrotuzumab, 4G5, OTMX005 and OTMX705. FAP is also a useful target in some fibrotic diseases.
Fibroblast growth factor receptor 2 (FGFR2) found in gastric and gastroesophageal junction cancers and adenocarcinomas and bound by bemarituzumab.
Fibronectin extra domain-B found on Hodgkin's lymphoma and bound by radretumab.fc
Folate receptor 1 found in epithelial-derived tumors including ovarian, breast, renal, lung (including non-small cell lung cancers and mesothelioma), colorectal, and brain and bound by farletuzumab and mirvetuximab.
Frizzled receptor (FZD1, 2, 5, 7, and 8 receptors) found on breast and pancreatic cancers, and cancer stem cells, and bound by vantictumab.
G protein-coupled receptor family C group 5-member D (GPRC5D) found in multiple myeloma and bound by talquetamab.
Ganglioside GD2 found on neuroblastoma and bound by dinutuximab and naxitamab.
Ganglioside GD3 found on malignant melanoma and small cell lung cancer and bound by ecromeximab and mitumomab.
Gelatinase B found in gastric and gastroesophageal junction cancers and adenocarcinomas and bound by andecaliximab.
Glutamate carboxypeptidase II found on prostate cancer and bound by capromab.
Glypican 3 found in hepatocellular carcinoma and bound by codrituzumab.
Guanate cyclase 2C (GUCY2C) found on pancreatic and other gastrointestinal cancers and bound by indusatumab.
Hepatocyte growth factor receptor mesenchymal-epithelial transition (MET) found in non-small cell lung cancer and bound by emibetuzumab, ficlatuzumab, onartuzumab, rilotumumab, and telisotuzumab.
Human epidermal growth factor receptor 2 (HER2, ErbB2) found on breast, ovarian, and stomach cancers, adenocarcinoma of the lung, and aggressive forms of uterine cancer, such as uterine serous endometrial carcinoma, and bound by DS-8201, ertumaxomab, gancotamab, margetuximab, pertuzumab, timigutuzumab, trastuzumab, and TRBSO7.
Human epidermal growth factor receptor B3 (ErbB3, HER3) found on breast, testicular, squamous and non-squamous non-small cell lung cancers and bound by duligotuzumab, elgemtumab, lumretuzumab, patritumab, serebantumab, zenocutuzumab.
Insulin-like growth factor 1 (IGF-1) found on solid tumors, including adrenocortical and small lung cell carcinomas and bound by cixutumumab, dalotuzumab, figitumumab, ganitumab, robatumumab, teprotumumab, and xentuzumab.
Insulin-like growth factor 2 (IGF-2) found on breast and liver cancers and other solid tumors and bound by dusigitumab and xentuzumab.
Integrin αvβ3 found in melanoma, prostate, ovarian and other cancers and bound by etaracizumab.
Integrin α₅β₁ found in solid tumors and bound by volociximab.
Killer-cell immunoglobulin-like receptor 2D (KIR2D) found on solid (including squamous cell carcinoma of the head and neck) and hematological cancers (including AML) and bound by lirilumab.
Lewis Y antigen found on lung, breast, colon, pancreatic, and other cancers and bound by cBR96 and C242 (nacolomab).
LIV-1 found on metastatic breast cancer as well as in melanoma, and prostate, ovarian, uterine, and cervical cancers and bound by ladiratuzumab.
Leucine-rich repeat containing 15 (LRRC15) found on tumor cells (including triple-negative breast cancer, non-small cell lung cancer, colorectal cancer) and cancer-associated fibroblasts and bound by samrotamab.
Mesothelin found in mesothelioma, lung cancer, ovarian cancer, and pancreatic cancer, and bound by amatuximab and anetumab.
Mucin 1 (MUC1) found in pancreatic, breast, and ovarian cancers and bound by clivatuzumab, gatipotuzumab, and pemtumomab.
Mucin 5AC (Muc5AC) found in colorectal and pancreatic carcinomas and bound by ensituximab.
Nectin 4 found in urothelial cancer and bound by enfortumab.
Notch 1 found in chemoresistant cancers and bound by brontictuzumab.
Notch 2/3 receptor found on pancreatic and lung cancers and bound by tarextumab.
PD-L1 found on urothelial carcinoma, non-small cell lung cancer (NSCLC), triple-negative breast cancer (TNBC), small cell lung cancer (SCLC), hepatocellular carcinoma (HCC), and melanoma and bound by atezolizumab, avelumab.
Phosphate-sodium co-transporter found on breast, thyroid, ovarian and non-small cell lung cancers and bound by lifastuzumab.
Platelet-derived growth factor receptor a (PDGF-Ra) found on solid tumors, particularly soft tissue sarcomas, glioblastoma, and non-small cell lung cancer, and bound by olaratumab and tovetumab. Also a useful target in some fibrotic diseases.
Prostate-specific membrane antigen (PSMA) found on prostate cancer and bound by pasotuxizumab.
PTK7 (tyrosine protein kinase-like 7) found on ovarian cancer, breast cancer, non-small cell lung and other cancers and bound by cofetuzumab.
Receptor activator of nuclear factor kappa-B ligand (RANKL) found in prostate and breast cancer (and bone metastases thereof) and multiple myeloma and bound by denosumab.
R-spondin 3 (RSPO3) found on solid tumors and bound by rosmantuzumab.
Six Transmembrane Epithelial Antigen of The Prostate 1 (STEAPI) found in prostate cancer and bound by vandortuzumab.
SLIT and NTRK-like protein 6 (SLITRK6) found on neural and brain tumor tissue and bound by sirtratumab.
Syndecan1 (SDC1; CD138) found on multiple myeloma and bound by indatuximab.
TRAIL-R1 found on multiple myeloma, and solid tumors including non-small cell lung cancer, colorectal cancer and liver cancer and bound by mapatumumab.
TRAIL-R2 found on pancreatic cancer, gastric, colorectal cancer, non-small cell lung cancer, cervical and ovarian cancer and bound by conatumumab, lexatumumab, and tigatuzumab.
Transmembrane glycoprotein NMB (GPNMB) found in melanoma and breast cancer and bound by glembatumumab.
Trophoblast glycoprotein (5T4) found on colorectal, ovarian, lung, renal, and gastric cancers and bound by naptumomab.
Tumor antigen CTAA16.88 found on colorectal tumors and bound by votumumab.
Tumor-associated calcium signal transducer 2 (also known as Trop-2) found on carcinomas, including triple negative breast cancer and metastatic urothelial caner, and bound by sacituzumab.
Tumor-associated glycoprotein 72 (TAG-72) found on breast, colon, lung, and pancreatic cancers and bound by anatumomab, minretumomab, and satumomab.
Tumor necrosis factor receptor superfamily member 12A (TWEAKR) found on solid tumors and bound by enavatuzumab.
Tyrosinase-related protein 1 (TYRP1) found in melanoma and bound by flanvotumab.
Tyrosine-protein kinase transmembrane receptor ROR1 found on chronic lymphocytic leukemia (CLL) and other cancers and bound by cirmtuzumab and zilovertamab.
Vimentin found on glioma and bound by pritumumab.

TABLE 3
Additional Exemplary Embodiments of Antibodies or Antigen-binding Portions

| | Antibody | Heavy Chain | Light Chain | | |
|---|---|---|---|---|---|
| Target | Name | SEQ ID NO: | SEQ ID NO: | Heavy Chain (V_{H}) (VL) | Light Chain (VL) |
| Activin | ascrinvacumab | 520 | 521 | QVQLQESGPGLVKPSQTLSLTCTVSGGSISSG | EIVLTQSPGTLSLSPGERATLSCRASQSVSS |
| | | | | EYYWNWIRQHPGKGLEWIGYIYYSGSTYYNP | SYLAWYQQKPGQAPRLLIYGTSSRATGIPDR |
| | | | | SLKSRVTISVDTSKNQFSLKLSSVTAADTAVYY | FSGSGSGTDFTLTISRLEPEDFAVYYCQQYG |
| | | | | CARESVAGFDYWGQGTLVTVSSASTKGPSVF | SSPITFGQGTRLEIKRTVAAPSVFIFPPSDEQL |
| | | | | PLAPCSRSTSESTAALGCLVKDYFPEPVTVSW | KSGTASVVCLLNNFYPREAKVQWKVDNALQ |
| | | | | NSGALTSGVHTFPAVLQSSGLYSLSSVVTVPS | SGNSQESVTEQDSKDSTYSLSSTLTLSKADY |
| | | | | SNFGTQTYTCNVDHKPSNTKVDKTVERKCCV | EKHKVYACEVTHQGLSSPVTKSFNRGEC |
| | | | | ECPPCPAPPVAGPSVFLFPPKPKDTLMISRTP | |
| | | | | EVTCVVVDVSHEDPEVQFNWYVDGVEVHNAK | |
| | | | | TKPREEQFNSTFRVVSVLTVVHQDWLNGKEY | |
| | | | | KCKVSNKGLPAPIEKTISKTKGQPREPQVYTLP | |
| | | | | PSREEMTKNQVSLTCLVKGFYPSDIAVEWESN | |
| | | | | GQPENNYKTTPPMLDSDGSFFLYSKLTVDKS | |
| | | | | RWQQGNVFSCSVMHEALHNHYTQKSLSLSP | |
| | | | | GK | |
| Alpha- | Tacatuzumab | 522 | 523 | QVQLQQSGAEVKKPGSSVKVSCKASGYAFTS | DIQLTQSPSSLSASVGDRVTITCKASQDINKY |
| fetoprotein | | | | YVIHWVRQAPGQGLYWIGYIHPYNGGTKYNE | IGWYQQKPGKAPKLLMHYTSALLPGIPSRFS |
| | | | | KFKGKATITADESTNTAYMELSSLRSEDTAFYF | GSGSGRDYTFTISSLQPEDIATYYCLQYDDL |
| | | | | CARSGGGDPFAYWGQGSLVTVSSASTKGPS | WTFGGGTKLQIKRTVAAPSVFIFPPSDEQLKS |
| | | | | VFPLAPSSKSTSGGTAALGCLVKDYFPEPVTV | GTASVVCLLNNFYPREAKVQWKVDNALQSG |
| | | | | SWNSGALTSGVHTFPAVLQSSGLYSLSSVVTV | NSQESVTEQDSKDSTYSLSSTLTLSKADYEK |
| | | | | PSSSLGTQTYICNVNHKPSNTKVDKRVEPKSC | HKVYACEVTHQGLSSPVTKSFNRGEC |
| | | | | DKTHTCPPCPAPELLGGPSVFLFPPKPKDTLM | |
| | | | | ISRTPEVTCVVVDVSHEDPEVKFNWYVDGVE | |
| | | | | VHNAKTKPREEQYNSTYRVVSVLTVLHQDWL | |
| | | | | NGKEYKCKVSNKALPAPIEKTISKAKGQPREP | |
| | | | | QVYTLPPSREEMTKNQVSLTCLVKGFYPSDIA | |
| | | | | VEWESNGQPENNYKTTPPVLDSDGSFFLYSK | |
| | | | | LTVDKSRWQQGNVFSCSVMHEALHNHYTQK | |
| | | | | SLSLSPGK | |
| AXL | Enapotamab | 524 | 525 | EVQLLESGGGLVQPGGSLRLSCAASGFTFSS | EIVLTQSPGTLSLSPGERATLSCRASQSVSS |
| receptor | | | | YAMNWVRQAPGKGLEWVSTTSGSGASTYYA | SYLAWYQQKPGQAPRLLIYGASSRATGIPDR |
| tyrosine | | | | DSVKGRFTISRDNSKNTLYLQMNSLRAEDTAV | FSGSGSGTDFTLTISRLEPEDFAVYYCQQYG |
| kinase | | | | YYCAKIWIAFDIWGQGTMVTVSSASTKGPSVF | SSPYTFGQGTKLEIKRTVAAPSVFIFPPSDEQ |
| | | | | PLAPSSKSTSGGTAALGCLVKDYFPEPVTVSW | LKSGTASVVCLLNNFYPREAKVQWKVDNALQ |
| | | | | NSGALTSGVHTFPAVLQSSGLYSLSSVVTVPS | SGNSQESVTEQDSKDSTYSLSSTLTLSKADY |
| | | | | SSLGTQTYICNVNHKPSNTKVDKRVEPKSCDK | EKHKVYACEVTHQGLSSPVTKSFNRGEC |
| | | | | THTCPPCPAPELLGGPSVFLFPPKPKDTLMIS | |
| | | | | RTPEVTCVVVDVSHEDPEVKFNWYVDGVEVH | |
| | | | | NAKTKPREEQYNSTYRVVSVLTVLHQDWLNG | |
| | | | | KEYKCKVSNKALPAPIEKTISKAKGQPREPQV | |
| | | | | YTLPPSREEMTKNQVSLTCLVKGFYPSDIAVE | |
| | | | | WESNGQPENNYKTTPPVLDSDGSFFLYSKLT | |
| | | | | VDKSRWQQGNVFSCSVMHEALHNHYTQKSL | |
| | | | | SLSPG | |
| CA125 | | 526 | 527 | EVQLVESGGGLVQPGGSLRLSCAASGYSITN | DIQMTQSPSSLSASVGDRVTITCKASDLIHN |
| | | | | DYAWNWVRQAPGKGLEWVGYISYSGYTTYN | WLAWYQQKPGKAPKLLIYGATSLETGVPSR |
| | | | | PSLKSRFTISRDTSKNTLYLQMNSLRAEDTAV | FSGSGSGTDFTLTISSLQPEDFATYYCQQYW |
| | | | | YYCARWTSGLDYWGQGTLVTVSSASTKGPSV | TTPFTFGQGTKVEIKRTVAAPSVFIFPPSDEQ |
| | | | | FPLAPSSKSTSGGTAALGCLVKDYFPEPVTVS | LKSGTASVVCLLNNFYPREAKVQWKVDNALQ |
| | | | | WNSGALTSGVHTFPAVLQSSGLYSLSSVVTVP | SGNSQESVTEQDSKDSTYSLSSTLTLSKADY |
| | | | | SSSLGTQTYICNVNHKPSNTKVDKKVEPKSCD | EKHKVYACEVTHQGLSSPVTKSFNRGEC |
| | | | | KTHTCPPCPAPELLGGPSVFLFPPKPKDTLMIS | |
| | | | | RTPEVTCVVVDVSHEDPEVKFNWYVDGVEVH | |
| | | | | NAKTKPREEQYNSTYRVVSVLTVLHQDWLNG | |
| | | | | KEYKCKVSNKALPAPIEKTISKAKGQPREPQV | |
| | | | | YTLPPSREEMTKNQVSLTCLVKGFYPSDIAVE | |
| | | | | WESNGQPENNYKTTPPVLDSDGSFFLYSKLT | |
| | | | | VDKSRWQQGNVFSCSVMHEALHNHYTQKSL | |
| | | | | SLSPGK | |
| CanAg | Cantuzumab | 528 | 529 | QVQLVQSGAEVKKPGETVKISCKASDYTFTYY | DIVMTQSPLSVPVTPGEPVSISCRSSKSLLHS |
| | | | | GMNWVKQAPGQGLKWMGWIDTTTGEPTYAQ | NGNTYLYWFLQRPGQSPQLLIYRMSNLVSG |
| | | | | KFQGRIAFSLETSASTAYLQIKSLKSEDTATYE | VPDRFSGSGSGTAFTLRISRVEAEDVGVYYC |
| | | | | CARRGPYNWYFDVWGQGTTVTVSSASTKGP | LQHLEYPFTFGPGTKLELKRTVAAPSVFIFPP |
| | | | | SVFPLAPSSKSTSGGTAALGCLVKDYFPEPVT | SDEQLKSGTASVVCLLNNFYPREAKVQWKV |
| | | | | VSWNSGALTSGVHTFPAVLQSSGLYSLSSVVT | DNALQSGNSQESVTEQDSKDSTYSLSSTLTL |
| | | | | VPSSSLGTQTYICNVNHKPSNTKVDKKVEPKS | SKADYEKHKVYACEVTHQGLSSPVTKSFNRG |
| | | | | CDKTHTCPPCPAPELLGGPSVFLFPPKPKDTL | EC |
| | | | | MISRTPEVTCVVVDVSHEDPEVKFNWYVDGV | |
| | | | | EVHNAKTKPREEQYNSTYRVVSVLTVLHQDW | |
| | | | | LNGKEYKCKVSNKALPAPIEKTISKAKGQPREP | |
| | | | | QVYTLPPSRDELTKNQVSLTCLVKGFYPSDIA | |
| | | | | VEWESNGQPENNYKTTPPVLDSDGSFFLYSK | |
| | | | | LTVDKSRWQQGNVFSCSVMHEALHNHYTQK | |
| | | | | SLSLSPGK | |
| Carbonic | girentuximab | 530 | 531 | DVKLVESGGGLVKLGGSLKLSCAASGFTFSNY | DIVMTQSQRFMSTTVGDRVSITCKASQNVVS |
| anhydrase | | | | YMSWVRQTPEKRLELVAAINSDGGITYYLDTV | AVAWYQQKPGQSPKLLIYSASNRYTGVPDR |
| 9 | | | | KGRFTISRDNAKNTLYLQMSSLKSEDTALFYC | FTGSGSGTDFTLTISNMQSEDLADFFCQQYS |
| | | | | ARHRSGYFSMDYWGQGTSVTVSSASTKGPS | NYPWTFGGGTKLEIKRTVAAPSVFIFPPSDE |
| | | | | VFPLAPSSKSTSGGTAALGCLVKDYFPEPVTV | QLKSGTASVVCLLNNFYPREAKVQWKVDNAL |
| | | | | SWNSGALTSGVHTFPAVLQSSGLYSLSSVVTV | QSGNSQESVTEQDSKDSTYSLSSTLTLSKAD |
| | | | | PSSSLGTQTYICNVNHKPSNTKVDKKVEPKSC | YEKHKVYACEVTHQGLSSPVTKSFNRGEC |
| | | | | DKTHTCPPCPAPELLGGPSVFLFPPKPKDTLM | |
| | | | | ISRTPEVTCVVVDVSHEDPEVKFNWYVDGVE | |
| | | | | VHNAKTKPREEQYNSTYRVVSVLTVLHQDWL | |
| | | | | NGKEYKCKVSNKALPAPIEKTISKAKGQPREP | |
| | | | | QVYTLPPSRDELTKNQVSLTCLVKGFYPSDIA | |
| | | | | VEWESNGQPENNYKTTPPVLDSDGSFFLYSK | |
| | | | | LTVDKSRWQQGNVFSCSVMHEALHNHYTQK | |
| | | | | SLSLSPGK | |
| CEA | Arcitumomab | 532 | 533 | EVKLVESGGGLVQPGGSLRLSCATSGFTFTD | QTVLSQSPAILSASPGEKVTMTCRASSSVTYI |
| | | | | YYMNWVRQPPGKALEWLGFIGNKANGYTTEY | HWYQQKPGSSPKSWIYATSNLASGVPARFS |
| | | | | SASVKGRFTISRDKSQSILYLQMNTLRAEDSAT | GSGSGTSYSLTISRVEAEDAATYYCQHWSS |
| | | | | YYCTRDRGLRFYFDYWGQGTTLTVSSAKTTP | KPPTFGGGTKLEIKRADAAPTVSIFPPSSEQL |
| | | | | PSVYPLAPGSAAQTNSMVTLGCLVKGYFPEP | TSGGASVVCFLNNFYPKDINVKWKIDGSERQ |
| | | | | VTVTWNSGSLSSGVHTFPAVLQSDLYTLSSSV | NGVLNSWTDQDSKDSTYSMSSTLTLTKDEYE |
| | | | | TVPSSPRPSETVTCNVAHPASSTKVDKKIVPR | RHNSYTCEATHKTSTSPIVKSFNRNEC |
| | | | | DCPPCKCPAPNLLGGPSVFIFPPKIKDVLMISL | |
| | | | | SPIVTCVVVDVSEDDPDVQISWFVNNVEVHTA | |
| | | | | QTQTHREDYNSTLRVVSALPIQHQDWMSGKE | |
| | | | | FKCKVNNKDLPAPIERTISKPKGSVRAPQVYVL | |
| | | | | PPPEEEMTKKQVTLTCMVTDFMPEDIYVEWT | |
| | | | | NNGKTELNYKNTEPVLDSDGSYFMYSKLRVE | |
| | | | | KKNWVERNSYSCSVVHEGLHNHHTTKSFSR | |
| CEACAM5 | Cibisatamab | 534 | | QVQLVQSGAEVKKPGASVKVSCKASGYTFTE | |
| | heavy | | | FGMNWVRQAPGQGLEWMGWINTKTGEATYV | |
| | chain 1- | | | EEFKGRVTFTTDTSTSTAYMELRSLRSDDTAV | |
| | bispecific | | | YYCARWDFAYYVEAMDYWGQGTTVTVSSAT | |
| | CEACAM5 | | | KGPSVFPLAPSSKSTSGGTAALGCLVKDYFPE | |
| | and CD3E | | | PVTVSWNSGALTSGVHTFPAVLQSSGLYSLSS | |
| | antibody | | | VVTVPSSSLGTQTYICNVNHKPSNTKVDKKVE | |
| | | | | PKSCDGGGGSGGGGSEVQLLESGGGLVQPG | |
| | | | | GSLRLSCAASGFTFSTYAMNWVRQAPGKGLE | |
| | | | | WVSRIRSKYNNYATYYADSVKGRFTISRDDSK | |
| | | | | NTLYLQMNSLRAEDTAVYYCVRHGNFGNSYV | |
| | | | | SWFAYWGQGTLVTVSSASVAAPSVFIFPPSDE | |
| | | | | QLKSGTASVVCLLNNFYPREAKVQWKVDNAL | |
| | | | | QSGNSQESVTEQDSKDSTYSLSSTLTLSKADY | |
| | | | | EKHKVYACEVTHQGLSSPVTKSFNRGECDKT | |
| | | | | HTCPPCPAPEAAGGPSVFLFPPKPKDTLMISR | |
| | | | | TPEVTCVVVDVSHEDPEVKFNWYVDGVEVHN | |
| | | | | AKTKPREEQYNSTYRVVSVLTVLHQDWLNGK | |
| | | | | EYKCKVSNKALGAPIEKTISKAKGQPREPQVY | |
| | | | | TLPPCRDELTKNQVSLWCLVKGFYPSDIAVEW | |
| | | | | ESNGQPENNYKTTPPVLDSDGSFFLYSKLTVD | |
| | | | | KSRWQQGNVFSCSVMHEALHNHYTQKSLSLS | |
| | | | | PGK | |
| CEACAM5 | Cibisatamab | 535 | 536 | QVQLVQSGAEVKKPGASVKVSCKASGYTFTE | DIQMTQSPSSLSASVGDRVTITCKASAAVGT |
| | heavy | | | FGMNWVRQAPGQGLEWMGWINTKTGEATYV | YVAWYQQKPGKAPKLLIYSASYRKRGVPSR |
| | and light | | | EEFKGRVTFTTDTSTSTAYMELRSLRSDDTAV | FSGSGSGTDFTLTISSLQPEDFATYYCHQYY |
| | chain 2- | | | YYCARWDFAYYVEAMDYWGQGTTVTVSSAS | TYPLFTFGQGTKLEIKRTVAAPSVFIFPPSDE |
| | bispecific | | | TKGPSVFPLAPSSKSTSGGTAALGCLVKDYFP | QLKSGTASVVCLLNNFYPREAKVQWKVDNAL |
| | CEACAM5 | | | EPVTVSWNSGALTSGVHTFPAVLQSSGLYSLS | QSGNSQESVTEQDSKDSTYSLSSTLTLSKAD |
| | and CD3E | | | SVVTVPSSSLGTQTYICNVNHKPSNTKVDKKV | YEKHKVYACEVTHQGLSSPVTKSFNRGEC |
| | antibody | | | EPKSCDKTHTCPPCPAPEAAGGPSVFLFPPKP | |
| | | | | KDTLMISRTPEVTCVVVDVSHEDPEVKFNWYV | |
| | | | | DGVEVHNAKTKPREEQYNSTYRVVSVLTVLH | |
| | | | | QDWLNGKEYKCKVSNKALGAPIEKTISKAKGQ | |
| | | | | PREPQVCTLPPSRDELTKNQVSLSCAVKGFYP | |
| | | | | SDIAVEWESNGQPENNYKTTPPVLDSDGSFFL | |
| | | | | VSKLTVDKSRWQQGNVFSCSVMHEALHNHYT | |
| | | | | QKSLSLSPGK | |
| CEACAM5 | Labetuzumab | 835 | 836 | EVQLVESGGGVVQPGRSLRLSCSASGFDFTT | DIQLTQSPSSLSASVGDRVTITCKASQDVGT |
| | | | | YWMSWVRQAPGKGLEWIGEIHPDSSTINYAP | SVAWYQQKPGKAPKLLIYWTSTRHTGVPSR |
| | | | | SLKDRFTISRDNAKNTLFLQMDSLRPEDTGVY | FSGSGSGTDFTFTISSLQPEDIATYYCQQYSL |
| | | | | FCASLYFGFPWFAYWGQGTPVTVSSASTKGP | YRSFGQGTKVEIKRTVAAPSVFIFPPSDEQLK |
| | | | | SVFPLAPSSKSTSGGTAALGCLVKDYFPEPVT | SGTASVVCLLNNFYPREAKVQWKVDNALQS |
| | | | | VSWNSGALTSGVHTFPAVLQSSGLYSLSSVVT | GNSQESVTEQDSKDSTYSLSSTLTLSKADYE |
| | | | | VPSSSLGTQTYICNVNHKPSNTKVDKRVEPKS | KHKVYACEVTHQGLSSPVTKSFNRGEC |
| | | | | CDKTHTCPPCPAPELLGGPSVFLFPPKPKDTL | |
| | | | | MISRTPEVTCVVVDVSHEDPEVKFNWYVDGV | |
| | | | | EVHNAKTKPREEQYNSTYRVVSVLTVLHQDW | |
| | | | | LNGKEYKCKVSNKALPAPIEKTISKAKGQPREP | |
| | | | | QVYTLPPSREEMTKNQVSLTCLVKGFYPSDIA | |
| | | | | VEWESNGQPENNYKTTPPVLDSDGSFFLYSK | |
| | | | | LTVDKSRWQQGNVFSCSVMHEALHNHYTQK | |
| | | | | SLSLSPGK | |
| CEACAM5 | Tusamitamab | 837 | 838 | EVQLQESGPGLVKPGGSLSLSCAASGFVFSS | DIQMTQSPASLSASVGDRVTITCRASENIFSY |
| | | | | YDMSWVRQTPERGLEWVAYISSGGGITYAPS | LAWYQQKPGKSPKLLVYNTRTLAEGVPSRF |
| | | | | TVKGRFTVSRDNAKNTLYLQMNSLTSEDTAVY | SGSGSGTDFSLTISSLQPEDFATYYCQHHYG |
| | | | | YCAAHYFGSSGPFAYWGQGTLVTVSSASTKG | TPFTFGSGTKLEIKRTVAAPSVFIFPPSDEQL |
| | | | | PSVFPLAPSSKSTSGGTAALGCLVKDYFPEPV | KSGTASVVCLLNNFYPREAKVQWKVDNALQ |
| | | | | TVSWNSGALTSGVHTFPAVLQSSGLYSLSSVV | SGNSQESVTEQDSKDSTYSLSSTLTLSKADY |
| | | | | TVPSSSLGTQTYICNVNHKPSNTKVDKKVEPK | EKHKVYACEVTHQGLSSPVTKSFNRGEC |
| | | | | SCDKTHTCPPCPAPELLGGPSVFLFPPKPKDT | |
| | | | | LMISRTPEVTCVVVDVSHEDPEVKFNWYVDG | |
| | | | | VEVHNAKTKPREEQYNSTYRVVSVLTVLHQD | |
| | | | | WLNGKEYKCKVSNKALPAPIEKTISKAKGQPR | |
| | | | | EPQVYTLPPSRDELTKNQVSLTCLVKGFYPSD | |
| | | | | IAVEWESNGQPENNYKTTPPVLDSDGSFFLYS | |
| | | | | KLTVDKSRWQQGNVFSCSVMHEALHNHYTQ | |
| | | | | KSLSLSPG | |
| CCR4 | mogamulizumab | 537 | 538 | EVQLVESGGDLVQPGRSLRLSCAASGFIFSNY | DVLMTQSPLSLPVTPGEPASISCRSSRNIVHI |
| | | | | GMSWVRQAPGKGLEWVATISSASTYSYYPDS | NGDTYLEWYLQKPGQSPQLLIYKVSNRFSG |
| | | | | VKGRFTISRDNAKNSLYLQMNSLRVEDTALYY | VPDRFSGSGSGTDFTLKISRVEAEDVGVYYC |
| | | | | CGRHSDGNFAFGYWGQGTLVTVSSASTKGP | FQGSLLPWTFGQGTKVEIKRTVAAPSVFIFPP |
| | | | | SVFPLAPSSKSTSGGTAALGCLVKDYFPEPVT | SDEQLKSGTASVVCLLNNFYPREAKVQWKV |
| | | | | VSWNSGALTSGVHTFPAVLQSSGLYSLSSVVT | DNALQSGNSQESVTEQDSKDSTYSLSSTLTL |
| | | | | VPSSSLGTQTYICNVNHKPSNTKVDKKVEPKS | SKADYEKHKVYACEVTHQGLSSPVTKSFNRG |
| | | | | CDKTHTCPPCPAPELLGGPSVFLFPPKPKDTL | EC |
| | | | | MISRTPEVTCVVVDVSHEDPEVKFNWYVDGV | |
| | | | | EVHNAKTKPREEQYNSTYRVVSVLTVLHQDW | |
| | | | | LNGKEYKCKVSNKALPAPIEKTISKAKGQPREP | |
| | | | | QVYTLPPSRDELTKNQVSLTCLVKGFYPSDIA | |
| | | | | VEWESNGQPENNYKTTPPVLDSDGSFFLYSK | |
| | | | | LTVDKSRWQQGNVFSCSVMHEALHNHYTQK | |
| | | | | SLSLSPGK | |
| CCR5 | Leronlimab | 539 | 540 | EVQLVESGGGLVKPGGSLRLSCAASGYTFSN | DIVMTQSPLSLPVTPGEPASISCRSSQRLLSS |
| | | | | YWIGWVRQAPGKGLEWIGDIYPGGNYIRNNE | YGHTYLHWYLQKPGQSPQLLIYEVSNRFSG |
| | | | | KFKDKTTLSADTSKNTAYLQMNSLKTEDTAVY | VPDRFSGSGSGTDFTLKISRVEAEDVGVYYC |
| | | | | YCGSSFGSNYVFAWFTYWGQGTLVTVSSAST | SQSTHVPLTFGQGTKVEIKRTVAAPSVFIFPP |
| | | | | KGPSVFPLAPCSRSTSESTAALGCLVKDYFPE | SDEQLKSGTASVVCLLNNFYPREAKVQWKV |
| | | | | PVTVSWNSGALTSGVHTFPAVLQSSGLYSLSS | DNALQSGNSQESVTEQDSKDSTYSLSSTLTL |
| | | | | VVTVPSSSLGTKTYTCNVDHKPSNTKVDKRVE | SKADYEKHKVYACEVTHQGLSSPVTKSFNRG |
| | | | | SKYGPPCPSCPAPEFLGGPSVFLFPPKPKDTL | EC |
| | | | | MISRTPEVTCVVVDVSQEDPEVQFNWYVDGV | |
| | | | | EVHNAKTKPREEQFNSTYRVVSVLTVLHQDW | |
| | | | | LNGKEYKCKVSNKGLPSSIEKTISKAKGQPRE | |
| | | | | PQVYTLPPSQEEMTKNQVSLTCLVKGFYPSDI | |
| | | | | AVEWESNGQPENNYKTTPPVLDSDGSFFLYS | |
| | | | | RLTVDKSRWQEGNVFSCSVMHEALHNHYTQ | |
| | | | | KSLSLSLGK | |
| CD19 | blinatumomab- | 541 | | DIQLTQSPASLAVSLGQRATISCKASQSVDYD | |
| | scFv-scFv | | | GDSYLNWYQQIPGQPPKLLIYDASNLVSGIPP | |
| | anti-CD3E | | | RFSGSGSGTDFTLNIHPVEKVDAATYHCQQS | |
| | and anti- | | | TEDPWTFGGGTKLEIKGGGGGGGGSGGGG | |
| | CD19 | | | SQVQLQQSGAELVRPGSSVKISCKASGYAFS | |
| | | | | SYWMNWVKQRPGQGLEWIGQIWPGDGDTNY | |
| | | | | NGKFKGKATLTADESSSTAYMQLSSLASEDSA | |
| | | | | VYFCARRETTTVGRYYYAMDYWGQGTTVTVS | |
| | | | | SGGGGSDIKLQQSGAELARPGASVKMSCKTS | |
| | | | | GYTFTRYTMHWVKQRPGQGLEWIGYINPSRG | |
| | | | | YTNYNQKFKDKATLTTDKSSSTAYMQLSSLTS | |
| | | | | EDSAVYYCARYYDDHYCLDYWGQGTTLTVSS | |
| | | | | VEGGSGGSGGSGGSGGVDDIQLTQSPAIMSA | |
| | | | | SPGEKVTMTCRASSSVSYMNWYQQKSGTSP | |
| | | | | KRWIYDTSKVASGVPYRFSGSGSGTSYSLTIS | |
| | | | | SMEAEDAATYYCQQWSSNPLTFGAGTKLELK | |
| | | | | HHHHHH | |
| CD19 | coltuximab | 542 | 543 | QVQLVQPGAEVVKPGASVKLSCKTSGYTFTS | EIVLTQSPAIMSASPGERVTMTCSASSGVNY |
| | | | | NWMHWVKQAPGQGLEWIGEIDPSDSYTNYN | MHWYQQKPGTSPRRWIYDTSKLASGVPARF |
| | | | | QNFQGKAKLTVDKSTSTAYMEVSSLRSDDTA | SGSGSGTDYSLTISSMEPEDAATYYCHQRG |
| | | | | VYYCARGSNPYYYAMDYWGQGTSVTVSSAS | SYTFGGGTKLEIKRTVAAPSVFIFPPSDEQLK |
| | | | | TKGPSVFPLAPSSKSTSGGTAALGCLVKDYFP | SGTASVVCLLNNFYPREAKVQWKVDNALQS |
| | | | | EPVTVSWNSGALTSGVHTFPAVLQSSGLYSLS | GNSQESVTEQDSKDSTYSLSSTLTLSKADYE |
| | | | | SVVTVPSSSLGTQTYICNVNHKPSNTKVDKKV | KHKVYACEVTHQGLSSPVTKSFNRGEC |
| | | | | EPKSCDKTHTCPPCPAPELLGGPSVFLFPPKP | |
| | | | | KDTLMISRTPEVTCVVVDVSHEDPEVKFNWYV | |
| | | | | DGVEVHNAKTKPREEQYNSTYRVVSVLTVLH | |
| | | | | QDWLNGKEYKCKVSNKALPAPIEKTISKAKGQ | |
| | | | | PREPQVYTLPPSRDELTKNQVSLTCLVKGFYP | |
| | | | | SDIAVEWESNGQPENNYKTTPPVLDSDGSFFL | |
| | | | | YSKLTVDKSRWQQGNVFSCSVMHEALHNHYT | |
| | | | | QKSLSLSPGK | |
| CD19 | Denintuzumab | 544 | 545 | QVQLQESGPGLVKPSQTLSLTCTVSGGSISTS | EIVLTQSPATLSLSPGERATLSCSASSSVSY |
| | | | | GMGVGWIRQHPGKGLEWIGHIWWDDDKRYN | MHWYQQKPGQAPRLLIYDTSKLASGIPARFS |
| | | | | PALKSRVTISVDTSKNQFSLKLSSVTAADTAVY | GSGSGTDFTLTISSLEPEDVAVYYCFQGSVY |
| | | | | YCARMELWSYYFDYWGQGTLVTVSSASTKG | PFTFGQGTKLEIKRTVAAPSVFIFPPSDEQLK |
| | | | | PSVFPLAPSSKSTSGGTAALGCLVKDYFPEPV | SGTASVVCLLNNFYPREAKVQWKVDNALQS |
| | | | | TVSWNSGALTSGVHTFPAVLQSSGLYSLSSVV | GNSQESVTEQDSKDSTYSLSSTLTLSKADYE |
| | | | | TVPSSSLGTQTYICNVNHKPSNTKVDKKVEPK | KHKVYACEVTHQGLSSPVTKSFNRGEC |
| | | | | SCDKTHTCPPCPAPELLGGPSVFLFPPKPKDT | |
| | | | | LMISRTPEVTCVVVDVSHEDPEVKFNWYVDG | |
| | | | | VEVHNAKTKPREEQYNSTYRVVSVLTVLHQD | |
| | | | | WLNGKEYKCKVSNKALPAPIEKTISKAKGQPR | |
| | | | | EPQVYTLPPSRDELTKNQVSLTCLVKGFYPSD | |
| | | | | IAVEWESNGQPENNYKTTPPVLDSDGSFFLYS | |
| | | | | KLTVDKSRWQQGNVFSCSVMHEALHNHYTQ | |
| | | | | KSLSLSPGK | |
| CD19 | Duvortuxizu- | 546 | 547 | ENVLTQSPATLSVTPGEKATITCRASQSVSYM | QAVVTQEPSLTVSPGGTVTLTCRSSTGAVTT |
| | mab | | | HWYQQKPGQAPRLLIYDASNRASGVPSRFS | SNYANWVQQKPGQAPRGLIGGTNKRAPWTP |
| | bispecific | | | GSGSGTDHTLTISSLEAEDAATYYCFQGSVYP | ARFSGSLLGGKAALTITGAQAEDEADYYCAL |
| | anti-CD3E | | | FTFGQGTKLEIKGGGSGGGGEVQLVESGGGL | WYSNLWVFGGGTKLTVLGGGGSGGGGQVT |
| | and anti- | | | VQPGGSLRLSCAASGFTFSTYAMNWVRQAP | LRESGPALVKPTQTLTLTCTFSGFSLSTSGM |
| | CD19 | | | GKGLEWVGRIRSKYNNYATYYADSVKGRFTIS | GVGWIRQPPGKALEWLAHIWWDDDKRYNPA |
| | | | | RDDSKNSLYLQMNSLKTEDTAVYYCVRHGNF | LKSRLTISKDTSKNQVFLTMTNMDPVDTATYY |
| | | | | GNSYVSWFAYWGQGTLVTVSSASTKGEVAA | CARMELWSYYFDYWGQGTTVTVSSASTKGK |
| | | | | CEKEVAALEKEVAALEKEVAALEKGGGDKTHT | VAACKEKVAALKEKVAALKEKVAALKE |
| | | | | CPPCPAPEAAGGPSVFLFPPKPKDTLMISRTP | |
| | | | | EVTCVVVDVSHEDPEVKFNWYVDGVEVHNAK | |
| | | | | TKPREEQYNSTYRVVSVLTVLHQDWLNGKEY | |
| | | | | KCKVSNKALPAPIEKTISKAKGQPREPQVYTLP | |
| | | | | PSREEMTKNQVSLWCLVKGFYPSDIAVEWES | |
| | | | | NGQPENNYKTTPPVLDSDGSFFLYSKLTVDKS | |
| | | | | RWQQGNVFSCSVMHEALHNHYTQKSLSLSP | |
| | | | | GK | |
| CD19 | inebilizumab | 548 | 549 | EVQLVESGGGLVQPGGSLRLSCAASGFTFSS | EIVLTQSPDFQSVTPKEKVTITCRASESVDTF |
| | | | | SWMNWVRQAPGKGLEWVGRIYPGDGDTNYN | GISFMNWFQQKPDQSPKLLIHEASNQGSGV |
| | | | | VKFKGRFTISRDDSKNSLYLQMNSLKTEDTAV | PSRFSGSGSGTDFTLTINSLEAEDAATYYCQ |
| | | | | YYCARSGFITTVRDFDYWGQGTLVTVSSASTK | QSKEVPFTFGGGTKVEIKRTVAAPSVFIFPPS |
| | | | | GPSVFPLAPSSKSTSGGTAALGCLVKDYFPEP | DEQLKSGTASVVCLLNNFYPREAKVQWKVD |
| | | | | VTVSWNSGALTSGVHTFPAVLQSSGLYSLSSV | NALQSGNSQESVTEQDSKDSTYSLSSTLTLS |
| | | | | VTVPSSSLGTQTYICNVNHKPSNTKVDKRVEP | KADYEKHKVYACEVTHQGLSSPVTKSFNRGE |
| | | | | KSCDKTHTCPPCPAPELLGGPSVFLFPPKPKD | C |
| | | | | TLMISRTPEVTCVVVDVSHEDPEVKFNWYVD | |
| | | | | GVEVHNAKTKPREEQYNSTYRVVSVLTVLHQ | |
| | | | | DWLNGKEYKCKVSNKALPAPIEKTISKAKGQP | |
| | | | | REPQVYTLPPSREEMTKNQVSLTCLVKGFYPS | |
| | | | | DIAVEWESNGQPENNYKTTPPVLDSDGSFFLY | |
| | | | | SKLTVDKSRWQQGNVFSCSVMHEALHNHYT | |
| | | | | QKSLSLSPGK | |
| CD19 | loncastuximab | 550 | 551 | QVQLVQPGAEVVKPGASVKLSCKTSGYTFTS | EIVLTQSPAIMSASPGERVTMTCSASSGVNY |
| | | | | NWMHWVKQAPGQGLEWIGEIDPSDSYTNYN | MHWYQQKPGTSPRRWIYDTSKLASGVPARF |
| | | | | QNFQGKAKLTVDKSTSTAYMEVSSLRSDDTA | SGSGSGTSYSLTISSMEPEDAATYYCHQRGS |
| | | | | VYYCARGSNPYYYAMDYWGQGTSVTVSSAS | YTFGGGTKLEIKRTVAAPSVFIFPPSDEQLKS |
| | | | | TKGPSVFPLAPSSKSTSGGTAALGCLVKDYFP | GTASVVCLLNNFYPREAKVQWKVDNALQSG |
| | | | | EPVTVSWNSGALTSGVHTFPAVLQSSGLYSLS | NSQESVTEQDSKDSTYSLSSTLTLSKADYEK |
| | | | | SVVTVPSSSLGTQTYICNVNHKPSNTKVDKKV | HKVYACEVTHQGLSSPVTKSFNRGEC |
| | | | | EPKSCDKTHTCPPCPAPELLGGPSVFLFPPKP | |
| | | | | KDTLMISRTPEVTCVVVDVSHEDPEVKFNWYV | |
| | | | | DGVEVHNAKTKPREEQYNSTYRVVSVLTVLH | |
| | | | | QDWLNGKEYKCKVSNKALPAPIEKTISKAKGQ | |
| | | | | PREPQVYTLPPSREEMTKNQVSLTCLVKGFYP | |
| | | | | SDIAVEWESNGQPENNYKTTPPVLDSDGSFFL | |
| | | | | YSKLTVDKSRWQQGNVFSCSVMHEALHNHYT | |
| | | | | QKSLSLSPG | |
| CD19 | Tafasitamab | 552 | 553 | EVQLVESGGGLVKPGGSLKLSCAASGYTFTS | DIVMTQSPATLSLSPGERATLSCRSSKSLQN |
| | | | | YVMHWVRQAPGKGLEWIGYINPYNDGTKYNE | VNGNTYLYWFQQKPGQSPQLLIYRMSNLNS |
| | | | | KFQGRVTISSDKSISTAYMELSSLRSEDTAMY | GVPDRFSGSGSGTEFTLTISSLEPEDFAVYY |
| | | | | YCARGTYYYGTRVFDYWGQGTLVTVSSASTK | CMQHLEYPITFGAGTKLEIKRTVAAPSVFIFP |
| | | | | GPSVFPLAPSSKSTSGGTAALGCLVKDYFPEP | PSDEQLKSGTASVVCLLNNFYPREAKVQWKV |
| | | | | VTVSWNSGALTSGVHTFPAVLQSSGLYSLSSV | DNALQSGNSQESVTEQDSKDSTYSLSSTLTL |
| | | | | VTVPSSSLGTQTYICNVNHKPSNTKVDKKVEP | SKADYEKHKVYACEVTHQGLSSPVTKSFNRG |
| | | | | KSCDKTHTCPPCPAPELLGGPDVFLFPPKPKD | EC |
| | | | | TLMISRTPEVTCVVVDVSHEDPEVQFNWYVD | |
| | | | | GVEVHNAKTKPREEQFNSTFRVVSVLTVVHQ | |
| | | | | DWLNGKEYKCKVSNKALPAPEEKTISKTKGQP | |
| | | | | REPQVYTLPPSREEMTKNQVSLTCLVKGFYPS | |
| | | | | DIAVEWESNGQPENNYKTTPPMLDSDGSFFL | |
| | | | | YSKLTVDKSRWQQGNVFSCSVMHEALHNHYT | |
| | | | | QKSLSLSPGK | |
| CD20 | Ibritumomab | 855 | 856 | QVQLQQPGAELVKAGASVKMSCKASGYTFTS | QIVLSQSPAILSASPGEKVTMTCRASSSVSYI |
| | | | | YNMHWVKQTPGRGLEWIGAIYPGNGDTSYNQ | HWFQQKPGSSPKPWIYATSNLASGVPVRFS |
| | | | | KFKGKATLTADKSSSTAYMQLSSLTSEDSAVY | GSGSGTSYSLTISRVEAEDAATYYCQQWTS |
| | | | | YCARSTYYGGDWYFNVWGAGTTVTVSA | NPPTFGGGTKLEIK |
| CD20 | Obinutuzumab | 554 | 555 | QVQLVQSGAEVKKPGSSVKVSCKASGYAFSY | DIVMTQTPLSLPVTPGEPASISCRSSKSLLHS |
| | | | | SWINWVRQAPGQGLEWMGRIFPGDGDTDYN | NGITYLYWYLQKPGQSPQLLIYQMSNLVSGV |
| | | | | GKFKGRVTITADKSTSTAYMELSSLRSEDTAV | PDRFSGSGSGTDFTLKISRVEAEDVGVYYCA |
| | | | | YYCARNVFDGYWLVYWGQGTLVTVSSASTK | QNLELPYTFGGGTKVEIKRTVAAPSVFIFPPS |
| | | | | GPSVFPLAPSSKSTSGGTAALGCLVKDYFPEP | DEQLKSGTASVVCLLNNFYPREAKVQWKVD |
| | | | | VTVSWNSGALTSGVHTFPAVLQSSGLYSLSSV | NALQSGNSQESVTEQDSKDSTYSLSSTLTLS |
| | | | | VTVPSSSLGTQTYICNVNHKPSNTKVDKKVEP | KADYEKHKVYACEVTHQGLSSPVTKSFNRGE |
| | | | | KSCDKTHTCPPCPAPELLGGPSVFLFPPKPKD | C |
| | | | | TLMISRTPEVTCVVVDVSHEDPEVKFNWYVD | |
| | | | | GVEVHNAKTKPREEQYNSTYRVVSVLTVLHQ | |
| | | | | DWLNGKEYKCKVSNKALPAPIEKTISKAKGQP | |
| | | | | REPQVYTLPPSRDELTKNQVSLTCLVKGFYPS | |
| | | | | DIAVEWESNGQPENNYKTTPPVLDSDGSFFLY | |
| | | | | SKLTVDKSRWQQGNVFSCSVMHEALHNHYT | |
| | | | | QKSLSLSPGK | |
| CD20 | Ocaratuzumab | 556 | 557 | EVQLVQSGAEVKKPGESLKISCKGSGRTFTSY | EIVLTQSPGTLSLSPGERATLSCRASSSVPYI |
| | | | | NMHWVRQMPGKGLEWMGAIYPLTGDTSYNQ | HWYQQKPGQAPRLLIYATSALASGIPDRFSG |
| | | | | KSKLQVTISADKSISTAYLQWSSLKASDTAMYY | SGSGTDFTLTISRLEPEDFAVYYCQQWLSNP |
| | | | | CARSTYVGGDWQFDVWGKGTTVTVSSASTK | PTFGQGTKLEIKRTVAAPSVFIFPPSDEQLKS |
| | | | | GPSVFPLAPSSKSTSGGTAALGCLVKDYFPEP | GTASVVCLLNNFYPREAKVQWKVDNALQSG |
| | | | | VTVSWNSGALTSGVHTFPAVLQSSGLYSLSSV | NSQESVTEQDSKDSTYSLSSTLTLSKADYEK |
| | | | | VTVPSSSLGTQTYICNVNHKPSNTKVDKKVEP | HKVYACEVTHQGLSSPVTKSFNRGEC |
| | | | | KSCDKTHTCPPCPAPELLGGPSVFLFPPKIKD | |
| | | | | TLMISRTPEVTCVVVDVSHEDPEVKFNWYVD | |
| | | | | GVEVHNAKTKPREEQYNSTYRVVSVLTVLHQ | |
| | | | | DWLNGKEYKCKVSNKALPAPIEKTISKQKGQP | |
| | | | | REPQVYTLPPSRDELTKNQVSLTCLVKGFYPS | |
| | | | | DIAVEWESNGQPENNYKTTPPVLDSDGSFFLY | |
| | | | | SKLTVDKSRWQQGNVFSCSVMHEALHNHYT | |
| | | | | QKSLSLSPG | |
| CD20 | ocrelizumab | 558 | 559 | EVQLVESGGGLVQPGGSLRLSCAASGYTFTS | DIQMTQSPSSLSASVGDRVTITCRASSSVSY |
| | | | | YNMHWVRQAPGKGLEWVGAIYPGNGDTSYN | MHWYQQKPGKAPKPLIYAPSNLASGVPSRF |
| | | | | QKFKGRFTISVDKSKNTLYLQMNSLRAEDTAV | SGSGSGTDFTLTISSLQPEDFATYYCQQWSF |
| | | | | YYCARVVYYSNSYWYFDVWGQGTLVTVSSAS | NPPTFGQGTKVEIKRTVAAPSVFIFPPSDEQL |
| | | | | TKGPSVFPLAPSSKSTSGGTAALGCLVKDYFP | KSGTASVVCLLNNFYPREAKVQWKVDNALQ |
| | | | | EPVTVSWNSGALTSGVHTFPAVLQSSGLYSLS | SGNSQESVTEQDSKDSTYSLSSTLTLSKADY |
| | | | | SVVTVPSSSLGTQTYICNVNHKPSNTKVDKKV | EKHKVYACEVTHQGLSSPVTKSFNRGEC |
| | | | | EPKSCDKTHTCPPCPAPELLGGPSVFLFPPKP | |
| | | | | KDTLMISRTPEVTCVVVDVSHEDPEVKFNWYV | |
| | | | | DGVEVHNAKTKPREEQYNSTYRVVSVLTVLH | |
| | | | | QDWLNGKEYKCKVSNKALPAPIEKTISKAKGQ | |
| | | | | PREPQVYTLPPSREEMTKNQVSLTCLVKGFYP | |
| | | | | SDIAVEWESNGQPENNYKTTPPVLDSDGSFFL | |
| | | | | YSKLTVDKSRWQQGNVFSCSVMHEALHNHYT | |
| | | | | QKSLSLSPGK | |
| CD20 | Ofatumumab | 560 | 561 | EVQLVESGGGLVQPGRSLRLSCAASGFTFND | EIVLTQSPATLSLSPGERATLSCRASQSVSS |
| | | | | YAMHWVRQAPGKGLEWVSTISWNSGSIGYAD | YLAWYQQKPGQAPRLLIYDASNRATGIPARF |
| | | | | SVKGRFTISRDNAKKSLYLQMNSLRAEDTALY | SGSGSGTDFTLTISSLEPEDFAVYYCQQRSN |
| | | | | YCAKDIQYGNYYYGMDVWGQGTTVTVSSAST | WPITFGQGTRLEIKRTVAAPSVFIFPPSDEQL |
| | | | | KGPSVFPLAPSSKSTSGGTAALGCLVKDYFPE | KSGTASVVCLLNNFYPREAKVQWKVDNALQ |
| | | | | PVTVSWNSGALTSGVHTFPAVLQSSGLYSLSS | SGNSQESVTEQDSKDSTYSLSSTLTLSKADY |
| | | | | VVTVPSSSLGTQTYICNVNHKPSNTKVDKRVE | EKHKVYACEVTHQGLSSPVTKSFNRGEC |
| | | | | PKSCDKTHTCPPCPAPELLGGPSVFLFPPKPK | |
| | | | | DTLMISRTPEVTCVVVDVSHEDPEVKFNWYVD | |
| | | | | GVEVHNAKTKPREEQYNSTYRVVSVLTVLHQ | |
| | | | | DWLNGKEYKCKVSNKALPAPIEKTISKAKGQP | |
| | | | | REPQVYTLPPSREEMTKNQVSLTCLVKGFYPS | |
| | | | | DIAVEWESNGQPENNYKTTPPVLDSDGSFFLY | |
| | | | | SKLTVDKSRWQQGNVFSCSVMHEALHNHYT | |
| | | | | QKSLSLSPGK | |
| CD20 | Rituximab | 562 | 563 | QVQLQQPGAELVKPGASVKMSCKASGYTFTS | QIVLSQSPAILSASPGEKVTMTCRASSSVSYI |
| | | | | YNMHWVKQTPGRGLEWIGAIYPGNGDTSYNQ | HWFQQKPGSSPKPWIYATSNLASGVPVRFS |
| | | | | KFKGKATLTADKSSSTAYMQLSSLTSEDSAVY | GSGSGTSYSLTISRVEAEDAATYYCQQWTS |
| | | | | YCARSTYYGGDWYFNVWGAGTTVTVSAAST | NPPTFGGGTKLEIKRTVAAPSVFIFPPSDEQL |
| | | | | KGPSVFPLAPSSKSTSGGTAALGCLVKDYFPE | KSGTASVVCLLNNFYPREAKVQWKVDNALQ |
| | | | | PVTVSWNSGALTSGVHTFPAVLQSSGLYSLSS | SGNSQESVTEQDSKDSTYSLSSTLTLSKADY |
| | | | | VVTVPSSSLGTQTYICNVNHKPSNTKVDKKVE | EKHKVYACEVTHQGLSSPVTKSFNRGEC |
| | | | | PKSCDKTHTCPPCPAPELLGGPSVFLFPPKPK | |
| | | | | DTLMISRTPEVTCVVVDVSHEDPEVKFNWYVD | |
| | | | | GVEVHNAKTKPREEQYNSTYRVVSVLTVLHQ | |
| | | | | DWLNGKEYKCKVSNKALPAPIEKTISKAKGQP | |
| | | | | REPQVYTLPPSRDELTKNQVSLTCLVKGFYPS | |
| | | | | DIAVEWESNGQPENNYKTTPPVLDSDGSFFLY | |
| | | | | SKLTVDKSRWQQGNVFSCSVMHEALHNHYT | |
| | | | | QKSLSLSPGK | |
| CD20 | Ublituximab | 564 | 565 | QAYLQQSGAELVRPGASVKMSCKASGYTFTS | QIVLSQSPAILSASPGEKVTMTCRASSSVSY |
| | | | | YNMHWVKQTPRQGLEWIGGIYPGNGDTSYN | MHWYQQKPGSSPKPWIYATSNLASGVPARF |
| | | | | QKFKGKATLTVGKSSSTAYMQLSSLTSEDSAV | SGSGSGTSYSFTISRVEAEDAATYYCQQWTF |
| | | | | YFCARYDYNYAMDYWGQGTSVTVSSASTKG | NPPTFGGGTRLEIKRTVAAPSVFIFPPSDEQL |
| | | | | PSVFPLAPSSKSTSGGTAALGCLVKDYFPEPV | KSGTASVVCLLNNFYPREAKVQWKVDNALQ |
| | | | | TVSWNSGALTSGVHTFPAVLQSSGLYSLSSVV | SGNSQESVTEQDSKDSTYSLSSTLTLSKADY |
| | | | | TVPSSSLGTQTYICNVNHKPSNTKVDKKVEPK | EKHKVYACEVTHQGLSSPVTKSFNRGEC |
| | | | | SCDKTHTCPPCPAPELLGGPSVFLFPPKPKDT | |
| | | | | LMISRTPEVTCVVVDVSHEDPEVKFNWYVDG | |
| | | | | VEVHNAKTKPREEQYNSTYRVVSVLTVLHQD | |
| | | | | WLNGKEYKCKVSNKALPAPIEKTISKAKGQPR | |
| | | | | EPQVYTLPPSRDELTKNQVSLTCLVKGFYPSD | |
| | | | | IAVEWESNGQPENNYKTTPPVLDSDGSFFLYS | |
| | | | | KLTVDKSRWQQGNVFSCSVMHEALHNHYTQ | |
| | | | | KSLSLSPGK | |
| CD20 | Veltuzumab | 566 | 567 | QVQLQQSGAEVKKPGSSVKVSCKASGYTFTS | DIQLTQSPSSLSASVGDRVTMTCRASSSVSY |
| | | | | YNMHWVKQAPGQGLEWIGAIYPGMGDTSYN | IHWFQQKPGKAPKPWIYATSNLASGVPVRF |
| | | | | QKFKGKATLTADESTNTAYMELSSLRSEDTAF | SGSGSGTDYTFTISSLQPEDIATYYCQQWTS |
| | | | | YYCARSTYYGGDWYFDVWGQGTTVTVSSAS | NPPTFGGGTKLEIKRTVAAPSVFIFPPSDEQL |
| | | | | TKGPSVFPLAPSSKSTSGGTAALGCLVKDYFP | KSGTASVVCLLNNFYPREAKVQWKVDNALQ |
| | | | | EPVTVSWNSGALTSGVHTFPAVLQSSGLYSLS | SGNSQESVTEQDSKDSTYSLSSTLTLSKADY |
| | | | | SVVTVPSSSLGTQTYICNVNHKPSNTKVDKRV | EKHKVYACEVTHQGLSSPVTKSFNRGEC |
| | | | | EPKSCDKTHTCPPCPAPELLGGPSVFLFPPKP | |
| | | | | KDTLMISRTPEVTCVVVDVSHEDPEVKFNWYV | |
| | | | | DGVEVHNAKTKPREEQYNSTYRVVSVLTVLH | |
| | | | | QDWLNGKEYKCKVSNKALPAPIEKTISKAKGQ | |
| | | | | PREPQVYTLPPSREEMTKNQVSLTCLVKGFYP | |
| | | | | SDIAVEWESNGQPENNYKTTPPVLDSDGSFFL | |
| | | | | YSKLTVDKSRWQQGNVFSCSVMHEALHNHYT | |
| | | | | QKSLSLSPGK | |
| CD20 | Mosunetuzu- | 568 | 569 | EVQLVESGGGLVQPGGSLRLSCAASGYTFTS | DIQMTQSPSSLSASVGDRVTITCRASSSVSY |
| | mab | | | YNMHWVRQAPGKGLEWVGAIYPGNGDTSYN | MHWYQQKPGKAPKPLIYAPSNLASGVPSRF |
| | bispecific | | | QKFKGRFTISVDKSKNTLYLQMNSLRAEDTAV | SGSGSGTDFTLTISSLQPEDFATYYCQQWSF |
| | anti-CD3 | | | YYCARVVYYSNSYWYFDVWGQGTLVTVSSAS | NPPTFGQGTKVEIKRTVAAPSVFIFPPSDEQL |
| | and anti- | | | TKGPSVFPLAPSSKSTSGGTAALGCLVKDYFP | KSGTASVVCLLNNFYPREAKVQWKVDNALQ |
| | CD20 | | | EPVTVSWNSGALTSGVHTFPAVLQSSGLYSLS | SGNSQESVTEQDSKDSTYSLSSTLTLSKADY |
| | antibody | | | SVVTVPSSSLGTQTYICNVNHKPSNTKVDKKV | EKHKVYACEVTHQGLSSPVTKSFNRGEC |
| | | | | EPKSCDKTHTCPPCPAPELLGGPSVFLFPPKP | |
| | | | | KDTLMISRTPEVTCVVVDVSHEDPEVKFNWYV | |
| | | | | DGVEVHNAKTKPREEQYGSTYRVVSVLTVLH | |
| | | | | QDWLNGKEYKCKVSNKALPAPIEKTISKAKGQ | |
| | | | | PREPQVYTLPPSREEMTKNQVSLWCLVKGFY | |
| | | | | PSDIAVEWESNGQPENNYKTTPPVLDSDGSF | |
| | | | | FLYSKLTVDKSRWQQGNVFSCSVMHEALHNH | |
| | | | | YTQKSLSLSPGK | |
| CD20 | Epcoritamab- | 570 | 571 | EVQLVESGGGLVQPDRSLRLSCAASGFTFHD | EIVLTQSPATLSLSPGERATLSCRASQSVSS |
| | bispecific | | | YAMHWVRQAPGKGLEWVSTISWNSGTIGYAD | YLAWYQQKPGQAPRLLIYDASNRATGIPARF |
| | anti-CD3 | | | SVKGRFTISRDNAKNSLYLQMNSLRAEDTALY | SGSGSGTDFTLTISSLEPEDFAVYYCQQRSN |
| | and anti- | | | YCAKDIQYGNYYYGMDVWGQGTTVTVSSAST | WPITFGQGTRLEIKRTVAAPSVFIFPPSDEQL |
| | CD20 | | | KGPSVFPLAPSSKSTSGGTAALGCLVKDYFPE | KSGTASVVCLLNNFYPREAKVQWKVDNALQ |
| | antibody | | | PVTVSWNSGALTSGVHTFPAVLQSSGLYSLSS | SGNSQESVTEQDSKDSTYSLSSTLTLSKADY |
| | | | | VVTVPSSSLGTQTYICNVNHKPSNTKVDKRVE | EKHKVYACEVTHQGLSSPVTKSFNRGEC |
| | | | | PKSCDKTHTCPPCPAPEFEGGPSVFLFPPKPK | |
| | | | | DTLMISRTPEVTCVVVAVSHEDPEVKFNWYVD | |
| | | | | GVEVHNAKTKPREEQYNSTYRVVSVLTVLHQ | |
| | | | | DWLNGKEYKCKVSNKALPAPIEKTISKAKGQP | |
| | | | | REPQVYTLPPSREEMTKNQVSLTCLVKGFYPS | |
| | | | | DIAVEWESNGQPENNYKTTPPVLDSDGSFFLY | |
| | | | | SRLTVDKSRWQQGNVFSCSVMHEALHNHYT | |
| | | | | QKSLSLSPG | |
| CD20 | Glofitamab | 572 | | QVQLVQSGAEVKKPGSSVKVSCKASGYAFSY | |
| | heavy | | | SWINWVRQAPGQGLEWMGRIFPGDGDTDYN | |
| | chain 1- | | | GKFKGRVTITADKSTSTAYMELSSLRSEDTAV | |
| | bispecific | | | YYCARNVFDGYWLVYWGQGTLVTVSSASTK | |
| | anti-CD3 | | | GPSVFPLAPSSKSTSGGTAALGCLVEDYFPEP | |
| | and anti- | | | VTVSWNSGALTSGVHTFPAVLQSSGLYSLSSV | |
| | CD20 | | | VTVPSSSLGTQTYICNVNHKPSNTKVDEKVEP | |
| | antibody | | | KSCDGGGGSGGGGSQAVVTQEPSLTVSPGG | |
| | | | | TVTLTCGSSTGAVTTSNYANWVQEKPGQAFR | |
| | | | | GLIGGTNKRAPGTPARFSGSLLGGKAALTLSG | |
| | | | | AQPEDEAEYYCALWYSNLWVFGGGTKLTVLS | |
| | | | | SASTKGPSVFPLAPSSKSTSGGTAALGCLVKD | |
| | | | | YFPEPVTVSWNSGALTSGVHTFPAVLQSSGL | |
| | | | | YSLSSVVTVPSSSLGTQTYICNVNHKPSNTKV | |
| | | | | DKKVEPKSCDKTHTCPPCPAPEAAGGPSVFL | |
| | | | | FPPKPKDTLMISRTPEVTCVVVDVSHEDPEVK | |
| | | | | FNWYVDGVEVHNAKTKPREEQYNSTYRVVSV | |
| | | | | LTVLHQDWLNGKEYKCKVSNKALGAPIEKTIS | |
| | | | | KAKGQPREPQVYTLPPCRDELTKNQVSLWCL | |
| | | | | VKGFYPSDIAVEWESNGQPENNYKTTPPVLD | |
| | | | | SDGSFFLYSKLTVDKSRWQQGNVFSCSVMHE | |
| | | | | ALHNHYTQKSLSLSPGK | |
| CD20 | Glofitamab | 573 | 574 | QVQLVQSGAEVKKPGSSVKVSCKASGYAFSY | DIVMTQTPLSLPVTPGEPASISCRSSKSLLHS |
| | heavy | | | SWINWVRQAPGQGLEWMGRIFPGDGDTDYN | NGITYLYWYLQKPGQSPQLLIYQMSNLVSGV |
| | chain 1- | | | GKFKGRVTITADKSTSTAYMELSSLRSEDTAV | PDRFSGSGSGTDFTLKISRVEAEDVGVYYCA |
| | bispecific | | | YYCARNVFDGYWLVYWGQGTLVTVSSASTK | QNLELPYTFGGGTKVEIKRTVAAPSVFIFPPS |
| | anti-CD3 | | | GPSVFPLAPSSKSTSGGTAALGCLVEDYFPEP | DRKLKSGTASVVCLLNNFYPREAKVQWKVD |
| | and anti- | | | VTVSWNSGALTSGVHTFPAVLQSSGLYSLSSV | NALQSGNSQESVTEQDSKDSTYSLSSTLTLS |
| | CD20 | | | VTVPSSSLGTQTYICNVNHKPSNTKVDEKVEP | KADYEKHKVYACEVTHQGLSSPVTKSFNRGE |
| | antibody | | | KSCDKTHTCPPCPAPEAAGGPSVFLFPPKPK | C |
| | | | | DTLMISRTPEVTCVVVDVSHEDPEVKFNWYVD | |
| | | | | GVEVHNAKTKPREEQYNSTYRVVSVLTVLHQ | |
| | | | | DWLNGKEYKCKVSNKALGAPIEKTISKAKGQP | |
| | | | | REPQVCTLPPSRDELTKNQVSLSCAVKGFYPS | |
| | | | | DIAVEWESNGQPENNYKTTPPVLDSDGSFFLV | |
| | | | | SKLTVDKSRWQQGNVFSCSVMHEALHNHYT | |
| | | | | QKSLSLSPGK | |
| CD20 | Odronextamab- | 575 | | EVQLVESGGGLVQPGRSLRLSCVASGFTFND | |
| | bispecific | | | YAMHWVRQAPGKGLEWVSVISWNSDSIGYAD | |
| | anti-CD3 | | | SVKGRFTISRDNAKNSLYLQMHSLRAEDTALY | |
| | and anti- | | | YCAKDNHYGSGSYYYYQYGMDVWGQGTTVT | |
| | CD20 | | | VSSASTKGPSVFPLAPCSRSTSESTAALGCLV | |
| | antibody | | | KDYFPEPVTVSWNSGALTSGVHTFPAVLQSS | |
| | | | | GLYSLSSVVTVPSSSLGTKTYTCNVDHKPSNT | |
| | | | | KVDKRVESKYGPPCPPCPAPPVAGPSVFLFP | |
| | | | | PKPKDTLMISRTPEVTCVVVDVSQEDPEVQFN | |
| | | | | WYVDGVEVHNAKTKPREEQFNSTYRVVSVLT | |
| | | | | VLHQDWLNGKEYKCKVSNKGLPSSIEKTISKA | |
| | | | | KGQPREPQVYTLPPSQEEMTKNQVSLTCLVK | |
| | | | | GFYPSDIAVEWESNGQPENNYKTTPPVLDSD | |
| | | | | GSFFLYSRLTVDKSRWQEGNVFSCSVMHEAL | |
| | | | | HNHYTQKSLSLSLGK | |
| CD22 | Epratuzumab- | 839 | 840 | QVQLVQSGAEVKKPGSSVKVSCKASGYTFTS | DIQLTQSPSSLSASVGDRVTMSCKSSQSVLY |
| | CD22 | | | YWLHWVRQAPGQGLEWIGYINPRNDYTEYNQ | SANHKNYLAWYQQKPGKAPKLLIYWASTRE |
| | binding | | | NFKDKATITADESTNTAYMELSSLRSEDTAFYF | SGVPSRFSGSGSGTDFTFTISSLQPEDIATYY |
| | fragment | | | CARRDITTFYWGQGTTVTVSS | CHQYLSSWTFGGGTKLEIK |
| CD22 | inotuzumab | 576 | 577 | QLVQSGAEVKKPGASVKVSCKASGYRFTNYW | DVQVTQSPSSLSASVGDRVTITCRSSQSLAN |
| | | | | IHWVRQAPGQGLEWIGGINPGNNYATYRRKE | SYGNTFLSWYLHKPGKAPQLLIYGISNRFSG |
| | | | | QGRVTMTADTSTSTVYMELSSLRSEDTAVYY | VPDRFSGSGSGTDFTLTISSLQPEDFATYYC |
| | | | | CTREGYGNYGAWFAYWGQGTLVTVSSASTK | LQGTHQPYTFGQGTKVEIKRTVAAPSVFIFPP |
| | | | | GPSVFPLAPCSRSTSESTAALGCLVKDYFPEP | SDEQLKSGTASVVCLLNNFYPREAKVQWKV |
| | | | | VTVSWNSGALTSGVHTFPAVLQSSGLYSLSSV | DNALQSGNSQESVTEQDSKDSTYSLSSTLTL |
| | | | | VTVPSSSLGTKTYTCNVDHKPSNTKVDKRVES | SKADYEKHKVYACEVTHQGLSSPVTKSFNRG |
| | | | | KYGPPCPPCPAPEFLGGPSVFLFPPKPKDTLM | EC |
| | | | | ISRTPEVTCVVVDVSQEDPEVQFNWYVDGVE | |
| | | | | VHNAKTKPREEQFNSTYRVVSVLTVLHQDWL | |
| | | | | NGKEYKCKVSNKGLPSSIEKTISKAKGQPREP | |
| | | | | QVYTLPPSQEEMTKNQVSLTCLVKGFYPSDIA | |
| | | | | VEWESNGQPENNYKTTPPVLDSDGSFFLYSR | |
| | | | | LTVDKSRWQEGNVFSCSVMHEALHNHYTQKS | |
| | | | | LSLSLGK | |
| CD22 | Moxetumomab- | 578 | 579 | MEVQLVESGGGLVKPGGSLKLSCAASGFAFSI | MDIQMTQTTSSLSASLGDRVTISCRASQDISN |
| | CD22 | | | YDMSWVRQTPEKCLEWVAYISSGGGTTYYPD | YLNWYQQKPDGTVKLLIYYTSILHSGVPSRF |
| | antibody | | | TVKGRFTISRDNAKNTLYLQMSSLKSEDTAMY | SGSGSGTDYSLTISNLEQEDFATYFCQQGNT |
| | fused to | | | YCARHSGYGTHWGVLFAYWGQGTLVTVSAK | LPWTFGCGTKLEIK |
| | LRP1 | | | ASGGPEGGSLAALTAHQACHLPLETFTRHRQ | |
| | | | | PRGWEQLEQCGYPVQRLVALYLAARLSWNQ | |
| | | | | VDQVIRNALASPGSGGDLGEAIREQPEQARLA | |
| | | | | LTLAAAESERFVRQGTGNDEAGAANGPADSG | |
| | | | | DALLERNYPTGAEFLGDGGDVSFSTRGTQNW | |
| | | | | TVERLLQAHRQLEERGYVFVGYHGTFLEAAQ | |
| | | | | SIVFGGVRARSQDLDAIWRGFYIAGDPALAYG | |
| | | | | YAQDQEPDARGRIRNGALLRVYVPRSSLPGF | |
| | | | | YRTSLTLAAPEAAGEVERLIGHPLPLRLDAITG | |
| | | | | PEEEGGRLETILGWPLAERTVVIPSAIPTDPRN | |
| | | | | VGGDLDPSSIPDKEQAISALPDYASQPGKPPR | |
| | | | | EDLK | |
| CD22 | Pinatuzumab | 580 | 581 | EVQLVESGGGLVQPGGSLRLSCAASGYEFSR | DIQMTQSPSSLSASVGDRVTITCRSSQSIVHS |
| | | | | SWMNWVRQAPGKGLEWVGRIYPGDGDTNYS | VGNTFLEWYQQKPGKAPKLLIYKVSNRFSG |
| | | | | GKFKGRFTISADTSKNTAYLQMNSLRAEDTAV | VPSRFSGSGSGTDFTLTISSLQPEDFATYYCF |
| | | | | YYCARDGSSWDWYFDVWGQGTLVTVSSAST | QGSQFPYTFGQGTKVEIKRTVAAPSVFIFPPS |
| | | | | KGPSVFPLAPSSKSTSGGTAALGCLVKDYFPE | DEQLKSGTASVVCLLNNFYPREAKVQWKVD |
| | | | | PVTVSWNSGALTSGVHTFPAVLQSSGLYSLSS | NALQSGNSQESVTEQDSKDSTYSLSSTLTLS |
| | | | | VVTVPSSSLGTQTYICNVNHKPSNTKVDKKVE | KADYEKHKVYACEVTHQGLSSPVTKSFNRGE |
| | | | | PKSCDKTHTCPPCPAPELLGGPSVFLFPPKPK | C |
| | | | | DTLMISRTPEVTCVVVDVSHEDPEVKFNWYVD | |
| | | | | GVEVHNAKTKPREEQYNSTYRVVSVLTVLHQ | |
| | | | | DWLNGKEYKCKVSNKALPAPIEKTISKAKGQP | |
| | | | | REPQVYTLPPSREEMTKNQVSLTCLVKGFYPS | |
| | | | | DIAVEWESNGQPENNYKTTPPVLDSDGSFFLY | |
| | | | | SKLTVDKSRWQQGNVFSCSVMHEALHNHYT | |
| | | | | QKSLSLSPGK | |
| CD23 | Lumiliximab- | 841 | 842 | EVQLVESGGGLAKPGGSLRLSCAASGFRFTF | DIQMTQSPSSLSASVGDRVTITCRASQDIRYY |
| | CD23 | | | NNYYMDWVRQAPGQGLEWVSRISSSGDPTW | LNWYQQKPGKAPKLLIYVASSLQSGVPSRF |
| | binding | | | YADSVKGRFTISRENANNTLFLQMNSLRAEDT | SGSGSGTEFTLTVSSLQPEDFATYYCLQVYS |
| | fragment | | | AVYYCASLTTGSDSWGQGVLVTVSS | TPRTFGQGTKVEIK |
| CD30 | brentuximab | 582 | 583 | QIQLQQSGPEVVKPGASVKISCKASGYTFTDY | DIVLTQSPASLAVSLGQRATISCKASQSVDF |
| | | | | YITWVKQKPGQGLEWIGWIYPGSGNTKYNEK | DGDSYMNWYQQKPGQPPKVLIYAASNLESG |
| | | | | FKGKATLTVDTSSSTAFMQLSSLTSEDTAVYE | IPARFSGSGSGTDFTLNIHPVEEEDAATYYC |
| | | | | CANYGNYWFAYWGQGTQVTVSAASTKGPSV | QQSNEDPWTFGGGTKLEIKRTVAAPSVFIFP |
| | | | | FPLAPSSKSTSGGTAALGCLVKDYFPEPVTVS | PSDEQLKSGTASVVCLLNNFYPREAKVQWKV |
| | | | | WNSGALTSGVHTFPAVLQSSGLYSLSSVVTVP | DNALQSGNSQESVTEQDSKDSTYSLSSTLTL |
| | | | | SSSLGTQTYICNVNHKPSNTKVDKKVEPKSCD | SKADYEKHKVYACEVTHQGLSSPVTKSFNRG |
| | | | | KTHTCPPCPAPELLGGPSVFLFPPKPKDTLMIS | EC |
| | | | | RTPEVTCVVVDVSHEDPEVKFNWYVDGVEVH | |
| | | | | NAKTKPREEQYNSTYRVVSVLTVLHQDWLNG | |
| | | | | KEYKCKVSNKALPAPIEKTISKAKGQPREPQV | |
| | | | | YTLPPSRDELTKNQVSLTCLVKGFYPSDIAVE | |
| | | | | WESNGQPENNYKTTPPVLDSDGSFFLYSKLT | |
| | | | | VDKSRWQQGNVFSCSVMHEALHNHYTQKSL | |
| | | | | SLSPG | |
| CD33 | lintuzumab | 584 | 585 | QVQLVQSGAEVKKPGSSVKVSCKASGYTFTD | DIQMTQSPSSLSASVGDRVTITCRASESVDN |
| | | | | YNMHWVRQAPGQGLEWIGYIYPYNGGTGYN | YGISFMNWFQQKPGKAPKLLIYAASNQGSG |
| | | | | QKFKSKATITADESTNTAYMELSSLRSEDTAV | VPSRFSGSGSGTDFTLTISSLQPDDFATYYC |
| | | | | YYCARGRPAMDYWGQGTLVTVSSASTKGPS | QQSKEVPWTFGQGTKVEIKRTVAAPSVFIFP |
| | | | | VFPLAPSSKSTSGGTAALGCLVKDYFPEPVTV | PSDEQLKSGTASVVCLLNNFYPREAKVQWKV |
| | | | | SWNSGALTSGVHTFPAVLQSSGLYSLSSVVTV | DNALQSGNSQESVTEQDSKDSTYSLSSTLTL |
| | | | | PSSSLGTQTYICNVNHKPSNTKVDKKVEPKSC | SKADYEKHKVYACEVTHQGLSSPVTKSFNRG |
| | | | | DKTHTCPPCPAPELLGGPSVFLFPPKPKDTLM | EC |
| | | | | ISRTPEVTCVVVDVSHEDPEVKFNWYVDGVE | |
| | | | | VHNAKTKPREEQYNSTYRVVSVLTVLHQDWL | |
| | | | | NGKEYKCKVSNKALPAPIEKTISKAKGQPREP | |
| | | | | QVYTLPPSRDELTKNQVSLTCLVKGFYPSDIA | |
| | | | | VEWESNGQPENNYKTTPPVLDSDGSFFLYSK | |
| | | | | LTVDKSRWQQGNVFSCSVMHEALHNHYTQK | |
| | | | | SLSLSPGK | |
| CD33 | Vadastuximab | 586 | 587 | QVQLVQSGAEVKKPGASVKVSCKASGYTFTN | DIQMTQSPSSLSASVGDRVTINCKASQDINS |
| | | | | YDINWVRQAPGQGLEWIGWIYPGDGSTKYNE | YLSWFQQKPGKAPKTLIYRANRLVDGVPSR |
| | | | | KFKAKATLTADTSTSTAYMELRSLRSDDTAVY | FSGSGSGQDYTLTISSLQPEDFATYYCLQYD |
| | | | | YCASGYEDAMDYWGQGTTVTVSSASTKGPS | EFPLTFGGGTKVEIKRTVAAPSVFIFPPSDEQ |
| | | | | VFPLAPSSKSTSGGTAALGCLVKDYFPEPVTV | LKSGTASVVCLLNNFYPREAKVQWKVDNALQ |
| | | | | SWNSGALTSGVHTFPAVLQSSGLYSLSSVVTV | SGNSQESVTEQDSKDSTYSLSSTLTLSKADY |
| | | | | PSSSLGTQTYICNVNHKPSNTKVDKKVEPKSC | EKHKVYACEVTHQGLSSPVTKSFNRGEC |
| | | | | DKTHTCPPCPAPELLGGPCVFLFPPKPKDTLM | |
| | | | | ISRTPEVTCVVVDVSHEDPEVKFNWYVDGVE | |
| | | | | VHNAKTKPREEQYNSTYRVVSVLTVLHQDWL | |
| | | | | NGKEYKCKVSNKALPAPIEKTISKAKGQPREP | |
| | | | | QVYTLPPSRDELTKNQVSLTCLVKGFYPSDIA | |
| | | | | VEWESNGQPENNYKTTPPVLDSDGSFFLYSK | |
| | | | | LTVDKSRWQQGNVFSCSVMHEALHNHYTQK | |
| | | | | SLSLSPGK | |
| CD33 | Gemtuzumab | 588 | 589 | EVQLVQSGAEVKKPGSSVKVSCKASGYTITDS | DIQLTQSPSTLSASVGDRVTITCRASESLDNY |
| | | | | NIHWVRQAPGQSLEWIGYIYPYNGGTDYNQK | GIRFLTWFQQKPGKAPKLLMYAASNQGSGV |
| | | | | FKNRATLTVDNPTNTAYMELSSLRSEDTAFYY | PSRFSGSGSGTEFTLTISSLQPDDFATYYCQ |
| | | | | CVNGNPWLAYWGQGTLVTVSSASTKGPSVFP | QTKEVPWSFGQGTKVEVKRTVAAPSVFIFPP |
| | | | | LAPCSRSTSESTAALGCLVKDYFPEPVTVSWN | SDEQLKSGTASVVCLLNNFYPREAKVQWKV |
| | | | | SGALTSGVHTFPAVLQSSGLYSLSSVVTVPSS | DNALQSGNSQESVTEQDSKDSTYSLSSTLTL |
| | | | | SLGTKTYTCNVDHKPSNTKVDKRVESKYGPP | SKADYEKHKVYACEVTHQGLSSPVTKSFNRG |
| | | | | CPPCPAPEFLGGPSVFLFPPKPKDTLMISRTP | EC |
| | | | | EVTCVVVDVSQEDPEVQFNWYVDGVEVHNA | |
| | | | | KTKPREEQFNSTYRVVSVLTVLHQDWLNGKE | |
| | | | | YKCKVSNKGLPSSIEKTISKAKGQPREPQVYTL | |
| | | | | PPSQEEMTKNQVSLTCLVKGFYPSDIAVEWES | |
| | | | | NGQPENNYKTTPPVLDSDGSFFLYSRLTVDKS | |
| | | | | RWQEGNVFSCSVMHEALHNHYTQKSLSLSLG | |
| | | | | K | |
| CD38 | Lilotomab | 590 | 591 | EIQLQQSGPELVKPGASVKVSCKASGYSFTDY | DIVMTQSHKLLSTSVGDRVSITCKASQDVST |
| | | | | NMYWVKQSHGKSLEWIGYIDPYNGDTTYNQK | AVDWYQQKPGQSPKLLINWASTRHTGVPDR |
| | | | | FKGKATLTVDKSSSTAFIHLNSLTSEDSAVYYC | FTGSGSGTDYTLTISSMQAEDLALYYCRQHY |
| | | | | ARSPYGHYAMDYWGQGTSVTVSSAKTTPPSV | STPFTFGSGTKLEIKRADAAPTVSIFPPSSEQ |
| | | | | YPLAPGSAAQTNSMVTLGCLVKGYFPEPVTVT | LTSGGASVVCFLNNFYPKDINVKWKIDGSER |
| | | | | WNSGSLSSGVHTFPAVLQSDLYTLSSSVTVPS | QNGVLNSWTDQDSKDSTYSMSSTLTLTKDE |
| | | | | STWPSETVTCNVAHPASSTKVDKKIVPRDCG | YERHNSYTCEATHKTSTSPIVKSFNRNEC |
| | | | | CKPCICTVPEVSSVFIFPPKPKDVLTITLTPKVT | |
| | | | | CVVVDISKDDPEVQFSWFVDDVEVHTAQTQP | |
| | | | | REEQFNSTFRSVSELPIMHQDWLNGKEFKCR | |
| | | | | VNSAAFPAPIEKTISKTKGRPKAPQVYTIPPPK | |
| | | | | EQMAKDKVSLTCMITDFFPEDITVEWQWNGQ | |
| | | | | PAENYKNTQPIMDTDGSYFVYSKLNVQKSNW | |
| | | | | EAGNTFTCSVLHEGLHNHHTEKSLSHSPGK | |
| CD37 | Naratuximab | 592 | 593 | QVQVQESGPGLVAPSQTLSITCTVSGFSLTTS | DIQMTQSPSSLSVSVGERVTITCRASENIRSN |
| | | | | GVSWVRQPPGKGLEWLGVIWGDGSTNYHPS | LAWYQQKPGKSPKLLVNVATNLADGVPSRF |
| | | | | LKSRLSIKKDHSKSQVFLKLNSLTAADTATYYC | SGSGSGTDYSLKINSLQPEDFGTYYCQHYW |
| | | | | AKGGYSLAHWGQGTLVTVSSASTKGPSVFPL | GTTWTFGQGTKLEIKRTVAAPSVFIFPPSDEQ |
| | | | | APSSKSTSGGTAALGCLVKDYFPEPVTVSWN | LKSGTASVVCLLNNFYPREAKVQWKVDNALQ |
| | | | | SGALTSGVHTFPAVLQSSGLYSLSSVVTVPSS | SGNSQESVTEQDSKDSTYSLSSTLTLSKADY |
| | | | | SLGTQTYICNVNHKPSNTKVDKKVEPKSCDKT | EKHKVYACEVTHQGLSSPVTKSFNRGEC |
| | | | | HTCPPCPAPELLGGPSVFLFPPKPKDTLMISR | |
| | | | | TPEVTCVVVDVSHEDPEVKFNWYVDGVEVHN | |
| | | | | AKTKPREEQYNSTYRVVSVLTVLHQDWLNGK | |
| | | | | EYKCKVSNKALPAPIEKTISKAKGQPREPQVYT | |
| | | | | LPPSRDELTKNQVSLTCLVKGFYPSDIAVEWE | |
| | | | | SNGQPENNYKTTPPVLDSDGSFFLYSKLTVDK | |
| | | | | SRWQQGNVFSCSVMHEALHNHYTQKSLSLSP | |
| | | | | G | |
| CD37 | Otlertuzumab- | 594 | | EVQLVQSGAEVKKPGESLKISCKGSGYSFTGY | |
| | heavy | | | NMNWVRQMPGKGLEWMGNIDPYYGGTTYNR | |
| | chain dimer | | | KFKGQVTISADKSISTAYLQWSSLKASDTAMY | |
| | | | | YCARSVGPFDSWGQGTLVTVSSGGGGSGGG | |
| | | | | GSGGGGSGGGGSGGGGSEIVLTQSPATLSL | |
| | | | | SPGERATLSCRASENVYSYLAWYQQKPGQA | |
| | | | | PRLLIYFAKTLAEGIPARFSGSGSGTDFTLTIS | |
| | | | | SLEPEDFAVYYCQHHSDNPWTFGQGTKVEIK | |
| | | | | GDQEPKSSDKTHTSPPCPAPELLGGPSVFLFP | |
| | | | | PKPKDTLMISRTPEVTCVVVDVSHEDPEVKFN | |
| | | | | WYVDGVEVHNAKTKPREEQYNSTYRVVSVLT | |
| | | | | VLHQDWLNGKEYKCKVSNKALPAPIEKTISKA | |
| | | | | KGQPREPQVYTLPPSRDELTKNQVSLTCLVK | |
| | | | | GFYPSDIAVEWESNGQPENNYKTTPPVLDSD | |
| | | | | GSFFLYSKLTVDKSRWQQGNVFSCSVMHEAL | |
| | | | | HNHYTQKSLSLSPGK | |
| CD37 | Ivicentamab- | 595 | 596 | EVQLVESGGGLVQPGGSLRLSCAASGFSLSN | DVVMTQSPSTLSASVGDRVTITCQASQNIDS |
| | biparatopic | | | YNMGWVRQAPGKGLEWVSVIDASGTTYYAT | NLAWYQQKPGKAPKFLIYYASNLPFGVPSR |
| | antibody | | | WAKGRFTISRDNSKNTLYLQMNSLRAEDTATY | FKGSGSGTEFTLTISSLQPDDFATYYCQSAD |
| | (Heavy/Light | | | YCARELLYFGSSYYDLWGQGTLVTVSSASTK | VGSTYVAAFGGGTKVEIKRTVAAPSVFIFPPS |
| | chain 1) | | | GPSVFPLAPSSKSTSGGTAALGCLVKDYFPEP | DEQLKSGTASVVCLLNNFYPREAKVQWKVD |
| | | | | VTVSWNSGALTSGVHTFPAVLQSSGLYSLSSV | NALQSGNSQESVTEQDSKDSTYSLSSTLTLS |
| | | | | VTVPSSSLGTQTYICNVNHKPSNTKVDKRVEP | KADYEKHKVYACEVTHQGLSSPVTKSFNRGE |
| | | | | KSCDKTHTCPPCPAPELLGGPSVFLFPPKPKD | C |
| | | | | TLMISRTPEVTCVVVDVSHEDPEVKFNWYVD | |
| | | | | GVEVHNAKTKPREEQYNSTYRVVSVLTVLHQ | |
| | | | | DWLNGKEYKCKVSNKALPAPIEKTISKAKGQP | |
| | | | | REPQVYTLPPSREEMTKNQVSLTCLVKGFYPS | |
| | | | | DIAVEWESNGQPENNYKTTPPVLDSDGSFLLY | |
| | | | | SKLTVDKSRWQQGNVFSCSVMHGALHNHYT | |
| | | | | QKSLSLSPG | |
| CD37 | Ivicentamab | 597 | 598 | EVQLVESGGGLVQPGGSLRLSCAASGFSLSY | AYDMTQSPSTLSASVGDRVTITCQASQNIIDY |
| | biparatopic | | | NAMNWVRQAPGKGLEWVSIIFASGRTDYASW | LAWYQQKPGKAPKLLIHKASTLASGVPSRF |
| | antibody | | | AKGRFTISRDNSKNTLYLQMNSLRAEDTAVYY | KGSGSGTEFTLTISSLQPDDFATYYCQQGYS |
| | (Heavy/Light | | | CAREGSTWGDALDPWGQGTLVTVSSASTKG | NSNIDNTFGGGTKVEIKRTVAAPSVFIFPPSD |
| | chain 2) | | | PSVFPLAPSSKSTSGGTAALGCLVKDYFPEPV | EQLKSGTASVVCLLNNFYPREAKVQWKVDN |
| | | | | TVSWNSGALTSGVHTFPAVLQSSGLYSLSSVV | ALQSGNSQESVTEQDSKDSTYSLSSTLTLSK |
| | | | | TVPSSSLGTQTYICNVNHKPSNTKVDKRVEPK | ADYEKHKVYACEVTHQGLSSPVTKSFNRGEC |
| | | | | SCDKTHTCPPCPAPELLGGPSVFLFPPKPKDT | |
| | | | | LMISRTPEVTCVVVDVSHEDPEVKFNWYVDG | |
| | | | | VEVHNAKTKPREEQYNSTYRVVSVLTVLHQD | |
| | | | | WLNGKEYKCKVSNKALPAPIEKTISKAKGQPR | |
| | | | | EPQVYTLPPSREEMTKNQVSLTCLVKGFYPSD | |
| | | | | IAVEWESNGQPENNYKTTPPVLDSDGSFFLYS | |
| | | | | RLTVDKSRWQQGNVFSCSVMHGALHNHYTQ | |
| | | | | KSLSLSPG | |
| CD38 | isatuximab | 599 | 600 | QVQLVQSGAEVAKPGTSVKLSCKASGYTFTD | DIVMTQSHLSMSTSLGDPVSITCKASQDVST |
| | | | | YWMQWVKQRPGQGLEWIGTIYPGDGDTGYA | VVAWYQQKPGQSPRRLIYSASYRYIGVPDRF |
| | | | | QKFQGKATLTADKSSKTVYMHLSSLASEDSAV | TGSGAGTDFTFTISSVQAEDLAVYYCQQHYS |
| | | | | YYCARGDYYGSNSLDYWGQGTSVTVSSASTK | PPYTFGGGTKLEIKRTVAAPSVFIFPPSDEQL |
| | | | | GPSVFPLAPSSKSTSGGTAALGCLVKDYFPEP | KSGTASVVCLLNNFYPREAKVQWKVDNALQ |
| | | | | VTVSWNSGALTSGVHTFPAVLQSSGLYSLSSV | SGNSQESVTEQDSKDSTYSLSSTLTLSKADY |
| | | | | VTVPSSSLGTQTYICNVNHKPSNTKVDKKVEP | EKHKVYACEVTHQGLSSPVTKSFNRGEC |
| | | | | KSCDKTHTCPPCPAPELLGGPSVFLFPPKPKD | |
| | | | | TLMISRTPEVTCVVVDVSHEDPEVKFNWYVD | |
| | | | | GVEVHNAKTKPREEQYNSTYRVVSVLTVLHQ | |
| | | | | DWLNGKEYKCKVSNKALPAPIEKTISKAKGQP | |
| | | | | REPQVYTLPPSRDELTKNQVSLTCLVKGFYPS | |
| | | | | DIAVEWESNGQPENNYKTTPPVLDSDGSFFLY | |
| | | | | SKLTVDKSRWQQGNVFSCSVMHEALHNHYT | |
| | | | | QKSLSLSPGK | |
| CD44 | Bivatuzumab- | 843 | 844 | EVQLVESGGGLVKPGGSLRLSCAASGFTFSS | EIVLTQSPATLSLSPGERATLSCSASSSINYIY |
| | CD44 | | | YDMSWVRQAPGKGLEWVSTISSGGSYTYYLD | WYQQKPGQAPRLLIYLTSNLASGVPARFSG |
| | binding | | | SIKGRFTISRDNAKNSLYLQMNSLRAEDTAVY | SGSGTDFTLTISSLEPEDFAVYYCLQWSSNP |
| | fragment | | | YCARQGLDYWGRGTLVTVSSA | LTFGGGTKVEIKR |
| CD51 | abituzumab | 601 | 602 | QVQLQQSGGELAKPGASVKVSCKASGYTESS | DIQMTQSPSSLSASVGDRVTITCRASQDISNY |
| | | | | FWMHWVRQAPGQGLEWIGYINPRSGYTEYN | LAWYQQKPGKAPKLLIYYTSKIHSGVPSRFS |
| | | | | EIFRDKATMTTDTSTSTAYMELSSLRSEDTAV | GSGSGTDYTFTISSLQPEDIATYYCQQGNTF |
| | | | | YYCASFLGRGAMDYWGQGTTVTVSSASTKG | PYTFGQGTKVEIKRTVAAPSVFIFPPSDEQLK |
| | | | | PSVFPLAPCSRSTSESTAALGCLVKDYFPEPV | SGTASVVCLLNNFYPREAKVQWKVDNALQS |
| | | | | TVSWNSGALTSGVHTFPAVLQSSGLYSLSSVV | GNSQESVTEQDSKDSTYSLSSTLTLSKADYE |
| | | | | TVPSSNFGTQTYTCNVDHKPSNTKVDKTVEP | KHKVYACEVTHQGLSSPVTKSFNRGEC |
| | | | | KSSDKTHTCPPCPAPPVAGPSVFLFPPKPKDT | |
| | | | | LMISRTPEVTCVVVDVSHEDPEVQFNWYVDG | |
| | | | | VEVHNAKTKPREEQAQSTFRVVSVLTVVHQD | |
| | | | | WLNGKEYKCKVSNKGLPAPIEKTISKTKGQPR | |
| | | | | EPQVYTLPPSREEMTKNQVSLTCLVKGFYPSD | |
| | | | | IAVEWESNGQPENNYKTTPPMLDSDGSFFLY | |
| | | | | SKLTVDKSRWQQGNVFSCSVMHEALHNHYT | |
| | | | | QKSLSLSPGK | |
| CD51 | Intetumumab | 603 | 604 | QVQLVESGGGVVQPGRSRRLSCAASGFTFS | EIVLTQSPATLSLSPGERATLSCRASQSVSS |
| | | | | RYTMHWVRQAPGKGLEWVAVISFDGSNKYY | YLAWYQQKPGQAPRLLIYDASNRATGIPARF |
| | | | | VDSVKGRFTISRDNSENTLYLQVNILRAEDTA | SGSGSGTDFTLTISSLEPEDFAVYYCQQRSN |
| | | | | VYYCAREARGSYAFDIWGQGTMVTVSSASTK | WPPFTFGPGTKVDIKRTVAAPSVFIFPPSDEQ |
| | | | | GPSVFPLAPSSKSTSGGTAALGCLVKDYFPEP | LKSGTASVVCLLNNFYPREAKVQWKVDNALQ |
| | | | | VTVSWNSGALTSGVHTFPAVLQSSGLYSLSSV | SGNSQESVTEQDSKDSTYSLSSTLTLSKADY |
| | | | | VTVPSSSLGTQTYICNVNHKPSNTKVDKKVEP | EKHKVYACEVTHQGLSSPVTKSFNRGEC |
| | | | | KSCDKTHTCPPCPAPELLGGPSVFLFPPKPKD | |
| | | | | TLMISRTPEVTCVVVDVSHEDPEVKFNWYVD | |
| | | | | GVEVHNAKTKPREEQYNSTYRVVSVLTVLHQ | |
| | | | | DWLNGKEYKCKVSNKALPAPIEKTISKAKGQP | |
| | | | | REPQVYTLPPSRDELTKNQVSLTCLVKGFYPS | |
| | | | | DIAVEWESNGQPENNYKTTPPVLDSDGSFFLY | |
| | | | | SKLTVDKSRWQQGNVFSCSVMHEALHNHYT | |
| | | | | QKSLSLSPGK | |
| CD52 | Alemtuzumab | 605 | 606 | QVQLQESGPGLVRPSQTLSLTCTVSGFTFTDF | DIQMTQSPSSLSASVGDRVTITCKASQNIDKY |
| | | | | YMNWVRQPPGRGLEWIGFIRDKAKGYTTEYN | LNWYQQKPGKAPKLLIYNTNNLQTGVPSRF |
| | | | | PSVKGRVTMLVDTSKNQFSLRLSSVTAADTAV | SGSGSGTDFTFTISSLQPEDIATYYCLQHISR |
| | | | | YYCAREGHTAAPFDYWGQGSLVTVSSASTKG | PRTFGQGTKVEIKRTVAAPSVFIFPPSDEQLK |
| | | | | PSVFPLAPSSKSTSGGTAALGCLVKDYFPEPV | SGTASVVCLLNNFYPREAKVQWKVDNALQS |
| | | | | TVSWNSGALTSGVHTFPAVLQSSGLYSLSSVV | GNSQESVTEQDSKDSTYSLSSTLTLSKADYE |
| | | | | TVPSSSLGTQTYICNVNHKPSNTKVDKKVEPK | KHKVYACEVTHQGLSSPVTKSFNRGEC |
| | | | | SCDKTHTCPPCPAPELLGGPSVFLFPPKPKDT | |
| | | | | LMISRTPEVTCVVVDVSHEDPEVKFNWYVDG | |
| | | | | VEVHNAKTKPREEQYNSTYRVVSVLTVLHQD | |
| | | | | WLNGKEYKCKVSNKALPAPIEKTISKAKGQPR | |
| | | | | EPQVYTLPPSRDELTKNQVSLTCLVKGFYPSD | |
| | | | | IAVEWESNGQPENNYKTTPPVLDSDGSFFLYS | |
| | | | | KLTVDKSRWQQGNVFSCSVMHEALHNHYTQ | |
| | | | | KSLSLSPGK | |
| CD52 | Gatralimab | 845 | 846 | VQLVESGGGLVQPGGSLRLSCAASGFPFSNY | DIVMTQTPLSLSVTPGQPASISCKSSQSLLYS |
| | | | | WMNWVRQAPGKGLEWVGQIRLKSNNYATHY | NGKTYLNWVLQKPGQSPQRLIYLVSKLDSG |
| | | | | AESVKGRFTISRDDSKNSLYLQMNSLKTEDTA | VPDRFSGSGSGTDFTLKISRVEAEDVGVYYC |
| | | | | VYYCTPIDYWGQGTTVTVSSASTKGPSVFPLA | VQGSHFHTFGQGTKLEIKRTVAAPSVFIFPPS |
| | | | | PSSKSTSGGTAALGCLVKDYFPEPVTVSWNS | DEQLKSGTASVVCLLNNFYPREAKVQWKVD |
| | | | | GALTSGVHTFPAVLQSSGLYSLSSVVTVPSSS | NALQSGNSQESVTEQDSKDSTYSLSSTLTLS |
| | | | | LGTQTYICNVNHKPSNTKVDKKVEPKSCDKTH | KADYEKHKVYACEVTHQGLSSPVTKSFNRGE |
| | | | | TCPPCPAPELLGGPSVFLFPPKPKDTLMISRTP | C |
| | | | | EVTCVVVDVSHEDPEVKFNWYVDGVEVHNAK | |
| | | | | TKPREEQYNSTYRVVSVLTVLHQDWLNGKEY | |
| | | | | KCKVSNKALPAPIEKTISKAKGQPREPQVYTLP | |
| | | | | PSRDELTKNQVSLTCLVKGFYPSDIAVEWESN | |
| | | | | GQPENNYKTTPPVLDSDGSFFLYSKLTVDKSR | |
| | | | | WQQGNVFSCSVMHEALHNHYTQKSLSLSPG | |
| | | | | K | |
| CD56 | Lorvotuzumab | 607 | 608 | QVQLVESGGGVVQPGRSLRLSCAASGFTFSS | DVVMTQSPLSLPVTLGQPASISCRSSQIIIHSD |
| | | | | FGMHWVRQAPGKGLEWVAYISSGSFTIYYAD | GNTYLEWFQQRPGQSPRRLIYKVSNRFSGV |
| | | | | SVKGRFTISRDNSKNTLYLQMNSLRAEDTAVY | PDRFSGSGSGTDFTLKISRVEAEDVGVYYCF |
| | | | | YCARMRKGYAMDYWGQGTLVTVSSASTKGP | QGSHVPHTFGQGTKVEIKRTVAAPSVFIFPPS |
| | | | | SVFPLAPSSKSTSGGTAALGCLVKDYFPEPVT | DEQLKSGTASVVCLLNNFYPREAKVQWKVD |
| | | | | VSWNSGALTSGVHTFPAVLQSSGLYSLSSVVT | NALQSGNSQESVTEQDSKDSTYSLSSTLTLS |
| | | | | VPSSSLGTQTYICNVNHKPSNTKVDKKVEPKS | KADYEKHKVYACEVTHQGLSSPVTKSFNRGE |
| | | | | CDKTHTCPPCPAPELLGGPSVFLFPPKPKDTL | C |
| | | | | MISRTPEVTCVVVDVSHEDPEVKFNWYVDGV | |
| | | | | EVHNAKTKPREEQYNSTYRVVSVLTVLHQDW | |
| | | | | LNGKEYKCKVSNKALPAPIEKTISKAKGQPREP | |
| | | | | QVYTLPPSRDELTKNQVSLTCLVKGFYPSDIA | |
| | | | | VEWESNGQPENNYKTTPPVLDSDGSFFLYSK | |
| | | | | LTVDKSRWQQGNVFSCSVMHEALHNHYTQK | |
| | | | | SLSLSPGK | |
| CD70 | cusatuzumab | 609 | 610 | EVQLVESGGGLVQPGGSLRLSCAASGFTFSV | QAVVTQEPSLTVSPGGTVTLTCGLKSGSVTS |
| | | | | YYMNWVRQAPGKGLEWVSDINNEGGTTYYA | DNFPTWYQQTPGQAPRLLIYNTNTRHSGVP |
| | | | | DSVKGRFTISRDNSKNSLYLQMNSLRAEDTAV | DRFSGSILGNKAALTITGAQADDEAEYFCAL |
| | | | | YYCARDAGYSNHVPIFDSWGQGTLVTVSSAS | FISNPSVEFGGGTQLTVLGQPKAAPSVTLFPP |
| | | | | TKGPSVFPLAPSSKSTSGGTAALGCLVKDYFP | SSEELQANKATLVCLISDFYPGAVTVAWKAD |
| | | | | EPVTVSWNSGALTSGVHTFPAVLQSSGLYSLS | SSPVKAGVETTTPSKQSNNKYAASSYLSLTP |
| | | | | SVVTVPSSSLGTQTYICNVNHKPSNTKVDKKV | EQWKSHRSYSCQVTHEGSTVEKTVAPTECS |
| | | | | EPKSCDKTHTCPPCPAPELLGGPSVFLFPPKP | |
| | | | | KDTLMISRTPEVTCVVVDVSHEDPEVKFNWYV | |
| | | | | DGVEVHNAKTKPREEQYNSTYRVVSVLTVLH | |
| | | | | QDWLNGKEYKCKVSNKALPAPIEKTISKAKGQ | |
| | | | | PREPQVYTLPPSRDELTKNQVSLTCLVKGFYP | |
| | | | | SDIAVEWESNGQPENNYKTTPPVLDSDGSFFL | |
| | | | | YSKLTVDKSRWQQGNVFSCSVMHEALHNHYT | |
| | | | | QKSLSLSPGK | |
| CD70 | Vorsetuzumab | 611 | 612 | QVQLVQSGAEVKKPGASVKVSCKASGYTFTN | DIVMTQSPDSLAVSLGERATINCRASKSVST |
| | | | | YGMNWVRQAPGQGLKWMGWINTYTGEPTYA | SGYSFMHWYQQKPGQPPKLLIYLASNLESG |
| | | | | DAFKGRVTMTRDTSISTAYMELSRLRSDDTAV | VPDRFSGSGSGTDFTLTISSLQAEDVAVYYC |
| | | | | YYCARDYGDYGMDYWGQGTTVTVSSASTKG | QHSREVPWTFGQGTKVEIKRTVAAPSVFIFP |
| | | | | PSVFPLAPSSKSTSGGTAALGCLVKDYFPEPV | PSDEQLKSGTASVVCLLNNFYPREAKVQWKV |
| | | | | TVSWNSGALTSGVHTFPAVLQSSGLYSLSSVV | DNALQSGNSQESVTEQDSKDSTYSLSSTLTL |
| | | | | TVPSSSLGTQTYICNVNHKPSNTKVDKKVEPK | SKADYEKHKVYACEVTHQGLSSPVTKSFNRG |
| | | | | SCDKTHTCPPCPAPELLGGPSVFLFPPKPKDT | EC |
| | | | | LMISRTPEVTCVVVDVSHEDPEVKFNWYVDG | |
| | | | | VEVHNAKTKPREEQYNSTYRVVSVLTVLHQD | |
| | | | | WLNGKEYKCKVSNKALPAPIEKTISKAKGQPR | |
| | | | | EPQVYTLPPSRDELTKNQVSLTCLVKGFYPSD | |
| | | | | IAVEWESNGQPENNYKTTPPVLDSDGSFFLYS | |
| | | | | KLTVDKSRWQQGNVFSCSVMHEALHNHYTQ | |
| | | | | KSLSLSPGK | |
| CD73 | Dresbuxelimab | 847 | 848 | QVQLVQSGAEVVKPGASVKVSCKASGYSFTG | DIVMTQSPSSLAVSVGERVTISCKSSQSLLNS |
| | | | | YTMNWVRQAPGQNLEWIGLINPYNAGTSYNQ | SNQKNYLAWYQQKPGQAPKLLIYFASTRES |
| | | | | KFQGKVTLTVDKSTSTAYMELSSLRSEDTAVY | GVPDRFSGSGSGTDFTLTISSLQAEDVAVYY |
| | | | | YCARSEYRYGGDYFDYWGQGTTLTVSSASTK | CQQHYDTPYTFGGGTKLEIKRTVAAPSVFIFP |
| | | | | GPSVFPLAPSSKSTSGGTAALGCLVKDYFPEP | PSDEQLKSGTASVVCLLNNFYPREAKVQWKV |
| | | | | VTVSWNSGALTSGVHTFPAVLQSSGLYSLSSV | DNALQSGNSQESVTEQDSKDSTYSLSSTLTL |
| | | | | VTVPSSSLGTQTYICNVNHKPSNTKVDKKVEP | SKADYEKHKVYACEVTHQGLSSPVTKSFNRG |
| | | | | KSCDKTHTCPPCPAPEAAGGPSVFLFPPKPK | EC |
| | | | | DTLMISRTPEVTCVVVDVSHEDPEVKFNWYVD | |
| | | | | GVEVHNAKTKPREEQYNSTYRVVSVLTVLHQ | |
| | | | | DWLNGKEYKCKVSNKALPAPIEKTISKAKGQP | |
| | | | | REPQVYTLPPSRDELTKNQVSLTCLVKGFYPS | |
| | | | | DIAVEWESNGQPENNYKTTPPVLDSDGSFFLY | |
| | | | | SKLTVDKSRWQQGNVFSCSVMHEALHNHYT | |
| | | | | QKSLSLSPGK | |
| CD73 | dalutrafusp | 849 | 850 | EVQLVQSGAEVKKPGESLKISCKASGYAFSSS | DIQMTQSPSSLSASVGDRVTITCRASQDISNY |
| | | | | WINWVRQMPGKGLEWMGRIYPRAGDTNYAG | LNWYQQKPGKAPKLLIYYTSRLHSGVPSRFS |
| | | | | KFKDQVTISADKSISTAYLQWSSLKASDTAMY | GSGSGTDFTFTISSLQPEDIATYYCQQGNTLP |
| | | | | YCASLLDYSMDYWGQGTLVTVSSASTKGPSV | LTFGQGTKVEIKRTVAAPSVFIFPPSDEQLKS |
| | | | | FPLAPSSKSTSGGTAALGCLVKDYFPEPVTVS | GTASVVCLLNNFYPREAKVQWKVDNALQSG |
| | | | | WNSGALTSGVHTFPAVLQSSGLYSLSSVVTVP | NSQESVTEQDSKDSTYSLSSTLTLSKADYEK |
| | | | | SSSLGTQTYICNVNHKPSNTKVDKRVEPKSCD | HKVYACEVTHQGLSSPVTKSFNRGEC |
| | | | | KTHTCPPCPAPELLGGPSVFLFPPKPKDTLMIS | |
| | | | | RTPEVTCVVVDVSHEDPEVKFNWYVDGVEVH | |
| | | | | NAKTKPREEQYASTYRVVSVLTVLHQDWLNG | |
| | | | | KEYKCKVSNKALPAPIEKTISKAKGQPREPQV | |
| | | | | YTLPPSREEMTKNQVSLTCLVKGFYPSDIAVE | |
| | | | | WESNGQPENNYKTTPPVLDSDGSFFLYSKLT | |
| | | | | VDKSRWQQGNVFSCSVMHEALHNHYTQKSL | |
| | | | | SLSPGGGGGSGGGGSGGGGSGGGGSGIPP | |
| | | | | HVQKSVNNDMIVTDNNGAVKFPQLCKFCDVR | |
| | | | | FSTCDNQKSCMSNCSITSICEKPQEVCVAVW | |
| | | | | RKNDENITLETVCHDPKLPYHDFILEDAASPKC | |
| | | | | IMKEKKKPGETFFMCSCSSDECNDNIIFSEEYN | |
| | | | | TSNPD | |
| CD73 | Oleclumab | 613 | 614 | EVQLLESGGGLVQPGGSLRLSCAASGFTFSS | QSVLTQPPSASGTPGQRVTISCSGSLSNIGR |
| | | | | YAYSWVRQAPGKGLEWVSAISGSGGRTYYAD | NPVNWYQQLPGTAPKLLIYLDNLRLSGVPDR |
| | | | | SVKGRFTISRDNSKNTLYLQMNSLRAEDTAVY | FSGSKSGTSASLAISGLQSEDEADYYCATW |
| | | | | YCARLGYGRVDEWGRGTLVTVSSASTKGPSV | DDSHPGWTFGGGTKLTVLGQPKAAPSVTLF |
| | | | | FPLAPSSKSTSGGTAALGCLVKDYFPEPVTVS | PPSSEELQANKATLVCLISDFYPGAVTVAWKA |
| | | | | WNSGALTSGVHTFPAVLQSSGLYSLSSVVTVP | DSSPVKAGVETTTPSKQSNNKYAASSYLSLT |
| | | | | SSSLGTQTYICNVNHKPSNTKVDKRVEPKSCD | PEQWKSHRSYSCQVTHEGSTVEKTVAPTEC |
| | | | | KTHTCPPCPAPEFEGGPSVFLFPPKPKDTLMI | S |
| | | | | SRTPEVTCVVVDVSHEDPEVKFNWYVDGVEV | |
| | | | | HNAKTKPREEQYNSTYRVVSVLTVLHQDWLN | |
| | | | | GKEYKCKVSNKALPASIEKTISKAKGQPREPQ | |
| | | | | VYTLPPSREEMTKNQVSLTCLVKGFYPSDIAV | |
| | | | | EWESNGQPENNYKTTPPVLDSDGSFFLYSKL | |
| | | | | TVDKSRWQQGNVFSCSVMHEALHNHYTQKS | |
| | | | | LSLSPGK | |
| CD74 | Milatuzumab | 615 | 616 | QVQLQQSGSELKKPGASVKVSCKASGYTFTN | DIQLTQSPLSLPVTLGQPASISCRSSQSLVHR |
| | | | | YGVNWIKQAPGQGLQWMGWINPNTGEPTFD | NGNTYLHWFQQRPGQSPRLLIYTVSNRFSG |
| | | | | DDFKGRFAFSLDTSVSTAYLQISSLKADDTAVY | VPDRFSGSGSGTDFTLKISRVEAEDVGVYFC |
| | | | | FCSRSRGKNEAWFAYWGQGTLVTVSSASTK | SQSSHVPPTFGAGTRLEIKRTVAAPSVFIFPP |
| | | | | GPSVFPLAPSSKSTSGGTAALGCLVKDYFPEP | SDEQLKSGTASVVCLLNNFYPREAKVQWKV |
| | | | | VTVSWNSGALTSGVHTFPAVLQSSGLYSLSSV | DNALQSGNSQESVTEQDSKDSTYSLSSTLTL |
| | | | | VTVPSSSLGTQTYICNVNHKPSNTKVDKRVEP | SKADYEKHKVYACEVTHQGLSSPVTKSFNRG |
| | | | | KSCDKTHTCPPCPAPELLGGPSVFLFPPKPKD | EC |
| | | | | TLMISRTPEVTCVVVDVSHEDPEVKFNWYVD | |
| | | | | GVEVHNAKTKPREEQYNSTYRVVSVLTVLHQ | |
| | | | | DWLNGKEYKCKVSNKALPAPIEKTISKAKGQP | |
| | | | | REPQVYTLPPSREEMTKNQVSLTCLVKGFYPS | |
| | | | | DIAVEWESNGQPENNYKTTPPVLDSDGSFFLY | |
| | | | | SKLTVDKSRWQQGNVFSCSVMHEALHNHYT | |
| | | | | QKSLSLSPGK | |
| CD79B | Iladatuzumab | 617 | 618 | EVQLVESGGGLVQPGGSLRLSCAASGYTFSS | DIQLTQSPSSLSASVGDRVTITCKASQSVDY |
| | | | | YWIEWVRQAPGKGLEWIGEILPGGGDTNYNEI | EGDSFLNWYQQKPGKAPKLLIYAASNLESG |
| | | | | FKGRATFSADTSKNTAYLQMNSLRAEDTAVYY | VPSRFSGSGSGTDFTLTISSLQPEDFATYYC |
| | | | | CTRRVPIRLDYWGQGTLVTVSSCSTKGPSVFP | QQSNEDPLTFGQGTKVEIKRTVAAPSVFIFPP |
| | | | | LAPSSKSTSGGTAALGCLVKDYFPEPVTVSW | SDEQLKSGTASVVCLLNNFYPREAKVQWKV |
| | | | | NSGALTSGVHTFPAVLQSSGLYSLSSVVTVPS | DNALQSGNSQESVTEQDSKDSTYSLSSTLTL |
| | | | | SSLGTQTYICNVNHKPSNTKVDKKVEPKSCDK | SKADYEKHKVYACEVTHQGLSSPVTKSFNRG |
| | | | | THTCPPCPAPELLGGPSVFLFPPKPKDTLMIS | EC |
| | | | | RTPEVTCVVVDVSHEDPEVKFNWYVDGVEVH | |
| | | | | NAKTKPREEQYNSTYRVVSVLTVLHQDWLNG | |
| | | | | KEYKCKVSNKALPAPIEKTISKAKGQPREPQV | |
| | | | | YTLPPSREEMTKNQVSLTCLVKGFYPSDIAVE | |
| | | | | WESNGQPENNYKTTPPVLDSDGSFFLYSKLT | |
| | | | | VDKSRWQQGNVFSCSVMHEALHNHYTQKSL | |
| | | | | SLSPGK | |
| CD79B | polatuzumab | 619 | 620 | EVQLVESGGGLVQPGGSLRLSCAASGYTFSS | DIQLTQSPSSLSASVGDRVTITCKASQSVDY |
| | | | | YWIEWVRQAPGKGLEWIGEILPGGGDTNYNEI | EGDSFLNWYQQKPGKAPKLLIYAASNLESG |
| | | | | FKGRATFSADTSKNTAYLQMNSLRAEDTAVYY | VPSRFSGSGSGTDFTLTISSLQPEDFATYYC |
| | | | | CTRRVPIRLDYWGQGTLVTVSSASTKGPSVFP | QQSNEDPLTFGQGTKVEIKRTVAAPSVFIFPP |
| | | | | LAPSSKSTSGGTAALGCLVKDYFPEPVTVSW | SDEQLKSGTASVVCLLNNFYPREAKVQWKV |
| | | | | NSGALTSGVHTFPAVLQSSGLYSLSSVVTVPS | DNALQSGNSQESVTEQDSKDSTYSLSSTLTL |
| | | | | SSLGTQTYICNVNHKPSNTKVDKKVEPKSCDK | SKADYEKHKVYACEVTHQGLSSPVTKSFNRG |
| | | | | THTCPPCPAPELLGGPSVFLFPPKPKDTLMIS | EC |
| | | | | RTPEVTCVVVDVSHEDPEVKFNWYVDGVEVH | |
| | | | | NAKTKPREEQYNSTYRVVSVLTVLHQDWLNG | |
| | | | | KEYKCKVSNKALPAPIEKTISKAKGQPREPQV | |
| | | | | YTLPPSREEMTKNQVSLTCLVKGFYPSDIAVE | |
| | | | | WESNGQPENNYKTTPPVLDSDGSFFLYSKLT | |
| | | | | VDKSRWQQGNVFSCSVMHEALHNHYTQKSL | |
| | | | | SLSPGK | |
| CD80 | Galiximab | 621 | 622 | QVQLQESGPGLVKPSETLSLTCAVSGGSISGG | ESALTQPPSVSGAPGQKVTISCTGSTSNIGG |
| | | | | YGWGWIRQPPGKGLEWIGSFYSSSGNTYYNP | YDLHWYQQLPGTAPKLLIYDINKRPSGISDRF |
| | | | | SLKSQVTISTDTSKNQFSLKLNSMTAADTAVY | SGSKSGTAASLAITGLQTEDEADYYCQSYDS |
| | | | | YCVRDRLFSVVGMVYNNWFDVWGPGVLVTV | SLNAQVFGGGTRLTVLGQPKAAPTVTLFPPS |
| | | | | SSASTKGPSVFPLAPSSKSTSGGTAALGCLVK | SEELQANKATLVCLISDFYPGAVTVAWKADS |
| | | | | DYFPEPVTVSWNSGALTSGVHTFPAVLQSSG | SPVKAGVETTTPSKQSNNKYAASSYLSLTPE |
| | | | | LYSLSSVVTVPSSSLGTQTYICNVNHKPSNTK | QWKSHRSYSCQVTHEGSTVEKTVAPTECS |
| | | | | VDKKAEPKSCDKTHTCPPCPAPELLGGPSVFL | |
| | | | | FPPKPKDTLMISRTPEVTCVVVDVSHEDPEVK | |
| | | | | FNWYVDGVEVHNAKTKPREEQYNSTYRVVSV | |
| | | | | LTVLHQDWLNGKEYKCKVSNKALPAPIEKTISK | |
| | | | | AKGQPREPQVYTLPPSRDELTKNQVSLTCLVK | |
| | | | | GFYPSDIAVEWESNGQPENNYKTTPPVLDSD | |
| | | | | GSFFLYSKLTVDKSRWQQGNVFSCSVMHEAL | |
| | | | | HNHYTQKSLSLSPGK | |
| CD123 | talacotuzumab | 623 | 624 | EVQLVQSGAEVKKPGESLKISCKGSGYSFTDY | DIVMTQSPDSLAVSLGERATINCESSQSLLN |
| | | | | YMKWARQMPGKGLEWMGDIIPSNGATFYNQ | SGNQKNYLTWYQQKPGQPPKPLIYWASTRE |
| | | | | KFKGQVTISADKSISTTYLQWSSLKASDTAMY | SGVPDRFSGSGSGTDFTLTISSLQAEDVAVY |
| | | | | YCARSHLLRASWFAYWGQGTMVTVSSASTK | YCQNDYSYPYTFGQGTKLEIKRTVAAPSVFIF |
| | | | | GPSVFPLAPSSKSTSGGTAALGCLVKDYFPEP | PPSDEQLKSGTASVVCLLNNFYPREAKVQWK |
| | | | | VTVSWNSGALTSGVHTFPAVLQSSGLYSLSSV | VDNALQSGNSQESVTEQDSKDSTYSLSSTLT |
| | | | | VTVPSSSLGTQTYICNVNHKPSNTKVDKKVEP | LSKADYEKHKVYACEVTHQGLSSPVTKSFNR |
| | | | | KSCDKTHTCPPCPAPELLGGPDVFLFPPKPKD | GEC |
| | | | | TLMISRTPEVTCVVVDVSHEDPEVQFNWYVD | |
| | | | | GVEVHNAKTKPREEQFNSTFRVVSVLTVVHQ | |
| | | | | DWLNGKEYKCKVSNKALPAPEEKTISKTKGQP | |
| | | | | REPQVYTLPPSREEMTKNQVSLTCLVKGFYPS | |
| | | | | DIAVEWESNGQPENNYKTTPPMLDSDGSFFL | |
| | | | | YSKLTVDKSRWQQGNVFSCSVMHEALHNHYT | |
| | | | | QKSLSLSPGK | |
| CD159 | monalizumab | 625 | 626 | QVQLVQSGAEVKKPGASVKVSCKASGYTFTS | DIQMTQSPSSLSASVGDRVTITCRASENIYSY |
| | | | | YWMNWVRQAPGQGLEWMGRIDPYDSETHYA | LAWYQQKPGKAPKLLIYNAKTLAEGVPSRF |
| | | | | QKLQGRVTMTTDTSTSTAYMELRSLRSDDTA | SGSGSGTDFTLTISSLQPEDFATYYCQHHYG |
| | | | | VYYCARGGYDFDVGTLYWFFDVWGQGTTVT | TPRTFGGGTKVEIKRTVAAPSVFIFPPSDEQL |
| | | | | VSSASTKGPSVFPLAPCSRSTSESTAALGCLV | KSGTASVVCLLNNFYPREAKVQWKVDNALQ |
| | | | | KDYFPEPVTVSWNSGALTSGVHTFPAVLQSS | SGNSQESVTEQDSKDSTYSLSSTLTLSKADY |
| | | | | GLYSLSSVVTVPSSSLGTKTYTCNVDHKPSNT | EKHKVYACEVTHQGLSSPVTKSFNRGEC |
| | | | | KVDKRVESKYGPPCPPCPAPEFLGGPSVFLFP | |
| | | | | PKPKDTLMISRTPEVTCVVVDVSQEDPEVQFN | |
| | | | | WYVDGVEVHNAKTKPREEQFNSTYRVVSVLT | |
| | | | | VLHQDWLNGKEYKCKVSNKGLPSSIEKTISKA | |
| | | | | KGQPREPQVYTLPPSQEEMTKNQVSLTCLVK | |
| | | | | GFYPSDIAVEWESNGQPENNYKTTPPVLDSD | |
| | | | | GSFFLYSRLTVDKSRWQEGNVFSCSVMHEAL | |
| | | | | HNHYTQKSLSLSLGK | |
| CD248 | ontuxizumab | 627 | 628 | QVQLQESGPGLVRPSQTLSLTCTASGYTFTDY | DIQMTQSPSSLSASVGDRVTITCRASQNVGT |
| | | | | VIHWVKQPPGRGLEWIGYINPYDDDTTYNQKE | AVAWLQQTPGKAPKLLIYSASNRYTGVPSR |
| | | | | KGRVTMLVDTSSNTAYLRLSSVTAEDTAVYYC | FSGSGSGTDYTFTISSLQPEDIATYYCQQYTN |
| | | | | ARRGNSYDGYFDYSMDYWGSGTPVTVSSAS | YPMYTFGQGTKVQIKRTVAAPSVFIFPPSDE |
| | | | | TKGPSVFPLAPSSKSTSGGTAALGCLVKDYFP | QLKSGTASVVCLLNNFYPREAKVQWKVDNAL |
| | | | | EPVTVSWNSGALTSGVHTFPAVLQSSGLYSLS | QSGNSQESVTEQDSKDSTYSLSSTLTLSKAD |
| | | | | SVVTVPSSSLGTQTYICNVNHKPSNTKVDKKV | YEKHKVYACEVTHQGLSSPVTKSFNRGEC |
| | | | | EPKSCDKTHTCPPCPAPELLGGPSVFLFPPKP | |
| | | | | KDTLMISRTPEVTCVVVDVSHEDPEVKFNWYV | |
| | | | | DGVEVHNAKTKPREEQYNSTYRVVSVLTVLH | |
| | | | | QDWLNGKEYKCKVSNKALPAPIEKTISKAKGQ | |
| | | | | PREPQVYTLPPSRDELTKNQVSLTCLVKGFYP | |
| | | | | SDIAVEWESNGQPENNYKTTPPVLDSDGFFFL | |
| | | | | YSKLTVDKSRWQQGNVFSCSVMHEALHNHYT | |
| | | | | QKSLSLSPGK | |
| CD276 | enoblituzumab | 629 | 630 | EVQLVESGGGLVQPGGSLRLSCAASGFTFSS | DIQLTQSPSFLSASVGDRVTITCKASQNVDT |
| | | | | FGMHWVRQAPGKGLEWVAYISSDSSAIYYAD | NVAWYQQKPGKAPKALIYSASYRYSGVPSR |
| | | | | TVKGRFTISRDNAKNSLYLQMNSLRDEDTAVY | FSGSGSGTDFTLTISSLQPEDFATYYCQQYN |
| | | | | YCGRGRENIYYGSRLDYWGQGTTVTVSSAST | NYPFTFGQGTKLEIKRTVAAPSVFIFPPSDEQ |
| | | | | KGPSVFPLAPSSKSTSGGTAALGCLVKDYFPE | LKSGTASVVCLLNNFYPREAKVQWKVDNALQ |
| | | | | PVTVSWNSGALTSGVHTFPAVLQSSGLYSLSS | SGNSQESVTEQDSKDSTYSLSSTLTLSKADY |
| | | | | VVTVPSSSLGTQTYICNVNHKPSNTKVDKRVE | EKHKVYACEVTHQGLSSPVTKSFNRGEC |
| | | | | PKSCDKTHTCPPCPAPELVGGPSVFLLPPKPK | |
| | | | | DTLMISRTPEVTCVVVDVSHEDPEVKFNWYVD | |
| | | | | GVEVHNAKTKPPEEQYNSTLRVVSVLTVLHQD | |
| | | | | WLNGKEYKCKVSNKALPAPIEKTISKAKGQPR | |
| | | | | EPQVYTLPPSREEMTKNQVSLTCLVKGFYPSD | |
| | | | | IAVEWESNGQPENNYKTTPLVLDSDGSFFLYS | |
| | | | | KLTVDKSRWQQGNVFSCSVMHEALHNHYTQ | |
| | | | | KSLSLSPGK | |
| CD276 | omburtamab | 631 | 632 | QVQLQQSGAELVKPGASVKLSCKASGYTFTN | DIVMTQSPATLSVTPGDRVSLSCRASQSISD |
| | | | | YDINWVRQRPEQGLEWIGWIFPGDGSTQYNE | YLHWYQQKSHESPRLLIKYASQSISGIPSRFS |
| | | | | KFKGKATLTTDTSSSTAYMQLSRLTSEDSAVY | GSGSGSDFTLSINSVEPEDVGVYYCQNGHSF |
| | | | | FCARQTTATWFAYWGQGTLVTVSAAKTTPPS | PLTFGAGTKLELKRADAAPTVSIFPPSSEQLT |
| | | | | VYPLAPGSAAQTNSMVTLGCLVKGYFPEPVTV | SGGASVVCFLNNFYPKDINVKWKIDGSERQN |
| | | | | TWNSGSLSSGVHTFPAVLQSDLYTLSSSVTVP | GVLNSWTDQDSKDSTYSMSSTLTLTKDEYER |
| | | | | SSTWPSETVTCNVAHPASSTKVDKKIVPRDCG | HNSYTCEATHKTSTSPIVKSFNRNEC |
| | | | | CKPCICTVPEVSSVFIFPPKPKDVLTITLTPKVT | |
| | | | | CVVVDISKDDPEVQFSWFVDDVEVHTAQTQP | |
| | | | | REEQFNSTFRSVSELPIMHQDWLNGKEFKCR | |
| | | | | VNSAAFPAPIEKTISKTKGRPKAPQVYTIPPPK | |
| | | | | EQMAKDKVSLTCMITDFFPEDITVEWQWNGQ | |
| | | | | PAENYKNTQPIMDTDGSYFVYSKLNVQKSNW | |
| | | | | EAGNTFTCSVLHEGLHNHHTEKSLSHSPGK | |
| CD319 | elotuzumab | 633 | 634 | EVQLVESGGGLVQPGGSLRLSCAASGFDFSR | DIQMTQSPSSLSASVGDRVTITCKASQDVGI |
| | | | | YWMSWVRQAPGKGLEWIGEINPDSSTINYAP | AVAWYQQKPGKVPKLLIYWASTRHTGVPDR |
| | | | | SLKDKFIISRDNAKNSLYLQMNSLRAEDTAVYY | FSGSGSGTDFTLTISSLQPEDVATYYCQQYS |
| | | | | CARPDGNYWYFDVWGQGTLVTVSSASTKGP | SYPYTFGQGTKVEIKRTVAAPSVFIFPPSDEQ |
| | | | | SVFPLAPSSKSTSGGTAALGCLVKDYFPEPVT | LKSGTASVVCLLNNFYPREAKVQWKVDNALQ |
| | | | | VSWNSGALTSGVHTFPAVLQSSGLYSLSSVVT | SGNSQESVTEQDSKDSTYSLSSTLTLSKADY |
| | | | | VPSSSLGTQTYICNVNHKPSNTKVDKKVEPKS | EKHKVYACEVTHQGLSSPVTKSFNRGEC |
| | | | | CDKTHTCPPCPAPELLGGPSVFLFPPKPKDTL | |
| | | | | MISRTPEVTCVVVDVSHEDPEVKFNWYVDGV | |
| | | | | EVHNAKTKPREEQYNSTYRVVSVLTVLHQDW | |
| | | | | LNGKEYKCKVSNKALPAPIEKTISKAKGQPREP | |
| | | | | QVYTLPPSRDELTKNQVSLTCLVKGFYPSDIA | |
| | | | | VEWESNGQPENNYKTTPPVLDSDGSFFLYSK | |
| | | | | LTVDKSRWQQGNVFSCSVMHEALHNHYTQK | |
| | | | | SLSLSPGK | |
| CD319 | Azintuxizumab | 635 | 636 | EVQLVESGGGLVQPGGSLRLSCAASGFTFSD | DVVMTQTPLSLSVTPGQPASISCRSSQSLVH |
| | | | | YYMAWVRQAPGKGLEWVASINYDGSSTYYVD | SNGNTYLHWYLQKPGQSPQLLIYKVSNRFS |
| | | | | SVKGRFTISRDNAKNSLYLQMNSLRAEDTAVY | GVPDRFSGSGSGTDFTLKISRVEAEDVGVYF |
| | | | | YCARDRGYYFDYWGQGTTVTVSSASTKGPSV | CSQSTHVPPFTFGGGTKVEIKRTVAAPSVFIF |
| | | | | FPLAPSSKSTSGGTAALGCLVKDYFPEPVTVS | PPSDEQLKSGTASVVCLLNNFYPREAKVQWK |
| | | | | WNSGALTSGVHTFPAVLQSSGLYSLSSVVTVP | VDNALQSGNSQESVTEQDSKDSTYSLSSTLT |
| | | | | SSSLGTQTYICNVNHKPSNTKVDKKVEPKSCD | LSKADYEKHKVYACEVTHQGLSSPVTKSFNR |
| | | | | KTHTCPPCPAPELLGGPSVFLFPPKPKDTLMIS | GEC |
| | | | | RTPEVTCVVVDVSHEDPEVKFNWYVDGVEVH | |
| | | | | NAKTKPREEQYNSTYRVVSVLTVLHQDWLNG | |
| | | | | KEYKCKVSNKALPAPIEKTISKAKGQPREPQV | |
| | | | | YTLPPSREEMTKNQVSLTCLVKGFYPSDIAVE | |
| | | | | WESNGQPENNYKTTPPVLDSDGSFFLYSKLT | |
| | | | | VDKSRWQQGNVFSCSVMHEALHNHYTQKSL | |
| | | | | SLSPGK | |
| Claudin-18 | Osemitamab | 851 | 852 | QVQLVQSGAEVKKPGASVKVSCKASGYTFTG | DIVMTQSPDSLAVSLGERATINCKSSQSLLN |
| isoform | | | | YNMNWVRQAPGQGLEWMGNIDPYYGGTSYN | SGNLKNYLTWYQQKPGQPPKLLIYWASTRK |
| 2 | | | | QKFKGRVTMTIDKSTSTVYMELSSLRSEDTAV | SGVPDRFSGSGSGTDFTLTISSLQAEDVAVY |
| | | | | YYCARMYHGNAFDYWGQGTTVTVSSASTKG | YCQNDYSYPLTFGGGTKVEIKRTVAAPSVFIF |
| | | | | PSVFPLAPSSKSTSGGTAALGCLVKDYFPEPV | PPSDEQLKSGTASVVCLLNNFYPREAKVQWK |
| | | | | TVSWNSGALTSGVHTFPAVLQSSGLYSLSSVV | VDNALQSGNSQESVTEQDSKDSTYSLSSTLT |
| | | | | TVPSSSLGTQTYICNVNHKPSNTKVDKKVEPK | LSKADYEKHKVYACEVTHQGLSSPVTKSFNR |
| | | | | SCDKTHTCPPCPAPELLGGPSVFLFPPKPKDT | GEC |
| | | | | LMISRTPEVTCVVVDVSHEDPEVKFNWYVDG | |
| | | | | VEVHNAKTKPREEQYNSTYRVVSVLTVLHQD | |
| | | | | WLNGKEYKCKVSNKALPAPIEKTISKAKGQPR | |
| | | | | EPQVYTLPPSRDELTKNQVSLTCLVKGFYPSD | |
| | | | | IAVEWESNGQPENNYKTTPPVLDSDGSFFLYS | |
| | | | | KLTVDKSRWQQGNVFSCSVMHEALHNHYTQ | |
| | | | | KSLSLSPGK | |
| Claudin-18 | Zolbetuximab | 637 | 640 | QVQLQQPGAELVRPGASVKLSCKASGYTFTS | DIVMTQSPSSLTVTAGEKVTMSCKSSQSLLN |
| isofirm | | | | YWINWVKQRPGQGLEWIGNIYPSDSYTNYNQ | SGNQKNYLTWYQQKPGQPPKLLIYWASTRE |
| 2 | | | | KFKDKATLTVDKSSSTAYMQLSSPTSEDSAVY | SGVPDRFTGSGSGTDFTLTISSVQAEDLAVY |
| | | | | YCTRSWRGNSFDYWGQGTTLTVSSASTKGP | YCQNDYSYPFTFGSGTKLEIKRTVAAPSVFIF |
| | | | | SVFPLAPSSKSTSGGTAALGCLVKDYFPEPVT | PPSDEQLKSGTASVVCLLNNFYPREAKVQWK |
| | | | | VSWNSGALTSGVHTFPAVLQSSGLYSLSSVVT | VDNALQSGNSQESVTEQDSKDSTYSLSSTLT |
| | | | | VPSSSLGTQTYICNVNHKPSNTKVDKRVEPKS | LSKADYEKHKVYACEVTHQGLSSPVTKSFNR |
| | | | | CDKTHTCPPCPAPELLGGPSVFLFPPKPKDTL | GEC |
| | | | | MISRTPEVTCVVVDVSHEDPEVKFNWYVDGV | |
| | | | | EVHNAKTKPREEQYNSTYRVVSVLTVLHQDW | |
| | | | | LNGKEYKCKVSNKALPAPIEKTISKAKGQPREP | |
| | | | | QVYTLPPSREEMTKNQVSLTCLVKGFYPSDIA | |
| | | | | VEWESNGQPENNYKTTPPVLDSDGSFFLYSK | |
| | | | | LTVDKSRWQQGNVFSCSVMHEALHNHYTQK | |
| | | | | SLSLSPGK | |
| CLEC12A | Tepoditamab- | 641 | | QVQLVQSGAEVKKPGASVKVSCKASGYTFTS | |
| | bispecfic | | | YYMHWVRQAPGQGLEWMGIINPSGGSTSYA | |
| | anti- | | | QKFQGRVTMTRDTSTSTVYMELSSLRSEDTA | |
| | CLEC12A | | | VYYCAKGTTGDWFDYWGQGTLVTVSSASTK | |
| | and anti- | | | GPSVFPLAPSSKSTSGGTAALGCLVKDYFPEP | |
| | CD3E | | | VTVSWNSGALTSGVHTFPAVLQSSGLYSLSSV | |
| | | | | VTVPSSSLGTQTYICNVNHKPSNTKVDKRVEP | |
| | | | | KSCDKTHTCPPCPAPELGRGPSVFLFPPKPKD | |
| | | | | TLMISRTPEVTCVVVDVSHEDPEVKFNWYVD | |
| | | | | GVEVHNAKTKPREEQYNSTYRVVSVLTVLHQ | |
| | | | | DWLNGKEYKCKVSNKALPAPIEKTISKAKGQP | |
| | | | | REPQVYTDPPSREEMTKNQVSLTCEVKGFYP | |
| | | | | SDIAVEWESNGQPENNYKTTPPVLDSDGSFFL | |
| | | | | YSKLTVDKSRWQQGNVFSCSVMHEALHNHYT | |
| | | | | QKSLSLSPG | |
| CXCR4 | ulocuplumab | 642 | 643 | EVQLVESGGGLVQPGGSLRLSCAAAGFTFSS | DIQMTQSPSSLSASVGDRVTITCRASQGISS |
| | | | | YSMNWVRQAPGKGLEWVSYISSRSRTIYYAD | WLAWYQQKPEKAPKSLIYAASSLQSGVPSR |
| | | | | SVKGRFTISRDNAKNSLYLQMNSLRDEDTAVY | FSGSGSGTDFTLTISSLQPEDFVTYYCQQYN |
| | | | | YCARDYGGQPPYYYYYGMDVWGQGTTVTVS | SYPRTFGQGTKVEIKRTVAAPSVFIFPPSDEQ |
| | | | | SASTKGPSVFPLAPCSRSTSESTAALGCLVKD | LKSGTASVVCLLNNFYPREAKVQWKVDNALQ |
| | | | | YFPEPVTVSWNSGALTSGVHTFPAVLQSSGL | SGNSQESVTEQDSKDSTYSLSSTLTLSKADY |
| | | | | YSLSSVVTVPSSSLGTKTYTCNVDHKPSNTKV | EKHKVYACEVTHQGLSSPVTKSFNRGEC |
| | | | | DKRVESKYGPPCPPCPAPEFLGGPSVFLFPPK | |
| | | | | PKDTLMISRTPEVTCVVVDVSQEDPEVQFNW | |
| | | | | YVDGVEVHNAKTKPREEQFNSTYRVVSVLTVL | |
| | | | | HQDWLNGKEYKCKVSNKGLPSSIEKTISKAKG | |
| | | | | QPREPQVYTLPPSQEEMTKNQVSLTCLVKGF | |
| | | | | YPSDIAVEWESNGQPENNYKTTPPVLDSDGS | |
| | | | | FFLYSRLTVDKSRWQEGNVFSCSVMHEALHN | |
| | | | | HYTQKSLSLSLG | |
| DLL3 | Rovalpituzumab | 644 | 645 | QVQLVQSGAEVKKPGASVKVSCKASGYTFTN | EIVMTQSPATLSVSPGERATLSCKASQSVSN |
| | | | | YGMNWVRQAPGQGLEWMGWINTYTGEPTYA | DVVWYQQKPGQAPRLLIYYASNRYTGIPARF |
| | | | | DDFKGRVTMTTDTSTSTAYMELRSLRSDDTA | SGSGSGTEFTLTISSLQSEDFAVYYCQQDYT |
| | | | | VYYCARIGDSSPSDYWGQGTLVTVSSASTKG | SPWTFGQGTKLEIKRTVAAPSVFIFPPSDEQL |
| | | | | PSVFPLAPSSKSTSGGTAALGCLVKDYFPEPV | KSGTASVVCLLNNFYPREAKVQWKVDNALQ |
| | | | | TVSWNSGALTSGVHTFPAVLQSSGLYSLSSVV | SGNSQESVTEQDSKDSTYSLSSTLTLSKADY |
| | | | | TVPSSSLGTQTYICNVNHKPSNTKVDKKVEPK | EKHKVYACEVTHQGLSSPVTKSFNRGEC |
| | | | | SCDKTHTCPPCPAPELLGGPSVFLFPPKPKDT | |
| | | | | LMISRTPEVTCVVVDVSHEDPEVKFNWYVDG | |
| | | | | VEVHNAKTKPREEQYNSTYRVVSVLTVLHQD | |
| | | | | WLNGKEYKCKVSNKALPAPIEKTISKAKGQPR | |
| | | | | EPQVYTLPPSRDELTKNQVSLTCLVKGFYPSD | |
| | | | | IAVEWESNGQPENNYKTTPPVLDSDGSFFLYS | |
| | | | | KLTVDKSRWQQGNVFSCSVMHEALHNHYTQ | |
| | | | | KSLSLSPG | |
| DLL4 | demcizumab | 646 | 647 | QVQLVQSGAEVKKPGASVKISCKASGYSFTAY | DIVMTQSPDSLAVSLGERATISCRASESVDN |
| | | | | YIHWVKQAPGQGLEWIGYISSYNGATNYNQKE | YGISFMKWFQQKPGQPPKLLIYAASNQGSG |
| | | | | KGRVTFTTDTSTSTAYMELRSLRSDDTAVYYC | VPDRFSGSGSGTDFTLTISSLQAEDVAVYYC |
| | | | | ARDYDYDVGMDYWGQGTLVTVSSASTKGPS | QQSKEVPWTFGGGTKVEIKRTVAAPSVFIFP |
| | | | | VFPLAPCSRSTSESTAALGCLVKDYFPEPVTV | PSDEQLKSGTASVVCLLNNFYPREAKVQWKV |
| | | | | SWNSGALTSGVHTFPAVLQSSGLYSLSSVVTV | DNALQSGNSQESVTEQDSKDSTYSLSSTLTL |
| | | | | PSSNFGTQTYTCNVDHKPSNTKVDKTVERKC | SKADYEKHKVYACEVTHQGLSSPVTKSFNRG |
| | | | | CVECPPCPAPPVAGPSVFLFPPKPKDTLMISR | EC |
| | | | | TPEVTCVVVDVSHEDPEVQFNWYVDGVEVHN | |
| | | | | AKTKPREEQFNSTFRVVSVLTVVHQDWLNGK | |
| | | | | EYKCKVSNKGLPAPIEKTISKTKGQPREPQVY | |
| | | | | TLPPSREEMTKNQVSLTCLVKGFYPSDIAVEW | |
| | | | | ESNGQPENNYKTTPPMLDSDGSFFLYSKLTV | |
| | | | | DKSRWQQGNVFSCSVMHEALHNHYTQKSLS | |
| | | | | LSPG | |
| DLL4 | enoticumab | 648 | 649 | QVQLVESGGGVVQPGRSLRLSCAASGFTFSS | EIVLTQSPATLSLSPGERATLSCRASQSVSS |
| | | | | YGMHWVRQAPGKGLEWVSFLWYDGTNKNYV | YLAWYQQKPGQAPRLLIYDASNRATGIPARF |
| | | | | ESVKGRFTISRDNSKNMLYLEMNSLRAEDTAV | SGSGSGTDFTLTISSLEPEDFAVYYCQHRSN |
| | | | | YYCARDHDFRSGYEGWFDPWGQGTLVTVSS | WPPTFGGGTKVEIKRTVAAPSVFIFPPSDEQL |
| | | | | ASTKGPSVFPLAPSSKSTSGGTAALGCLVKDY | KSGTASVVCLLNNFYPREAKVQWKVDNALQ |
| | | | | FPEPVTVSWNSGALTSGVHTFPAVLQSSGLY | SGNSQESVTEQDSKDSTYSLSSTLTLSKADY |
| | | | | SLSSVVTVPSSSLGTQTYICNVNHKPSNTKVD | EKHKVYACEVTHQGLSSPVTKSFNRGEC |
| | | | | KKVEPKSCDKTHTCPPCPAPELLGGPSVFLFP | |
| | | | | PKPKDTLMISRTPEVTCVVVDVSHEDPEVKFN | |
| | | | | WYVDGVEVHNAKTKPREEQYNSTYRVVSVLT | |
| | | | | VLHQDWLNGKEYKCKVSNKALPAPIEKTISKA | |
| | | | | KGQPREPQVYTLPPSRDELTKNQVSLTCLVK | |
| | | | | GFYPSDIAVEWESNGQPENNYKTTPPVLDSD | |
| | | | | GSFFLYSKLTVDKSRWQQGNVFSCSVMHEAL | |
| | | | | HNHYTQKSLSLSPG | |
| DLL4 | Navicixizumab- | 650 | | QVQLVQSGAEVKKPGASVKISCKASGYSFTAY | |
| | bispecific | | | YIHWVKQAPGQGLEWIGYISNYNRATNYNQKF | |
| | anti-DLL4 | | | KGRVTFTTDTSTSTAYMELRSLRSDDTAVYYC | |
| | and anti- | | | ARDYDYDVGMDYWGQGTLVTVSSASTKGPS | |
| | VEGF | | | VFPLAPCSRSTSESTAALGCLVKDYFPEPVTV | |
| | antibody | | | SWNSGALTSGVHTFPAVLQSSGLYSLSSVVTV | |
| | | | | PSSNFGTQTYTCNVDHKPSNTKVDKTVERKC | |
| | | | | CVECPPCPAPPVAGPSVFLFPPKPKDTLMISR | |
| | | | | TPEVTCVVVDVSHEDPEVQFNWYVDGVEVHN | |
| | | | | AKTKPREEQFNSTFRVVSVLTVVHQDWLNGK | |
| | | | | EYKCKVSNKGLPAPIEKTISKTKGQPREPQVY | |
| | | | | TLPPSREEMTKNQVSLTCLVEGFYPSDIAVEW | |
| | | | | ESNGQPENNYKTTPPMLDSDGSFFLYSELTV | |
| | | | | DKSRWQQGNVFSCSVMHEALHNHYTQKSLS | |
| | | | | LSPGK | |
| Egfl7 | parsatuzumab | 651 | 652 | EVQLVESGGGLVQPGGSLRLSCAASGYTFIDY | DIQMTQSPSSLSASVGDRVTITCRTSQSLVHI |
| | | | | YMNWVRQAPGKGLEWVGDINLDNSGTHYNQ | NAITYLHWYQQKPGKAPKLLIYRVSNRFSGV |
| | | | | KFKGRFTISRDKSKNTAYLQMNSLRAEDTAVY | PSRFSGSGSGTDFTLTISSLQPEDFATYYCG |
| | | | | YCAREGVYHDYDDYAMDYWGQGTLVTVSSA | QSTHVPLTFGQGTKVEIKRTVAAPSVFIFPPS |
| | | | | STKGPSVFPLAPSSKSTSGGTAALGCLVKDYF | DEQLKSGTASVVCLLNNFYPREAKVQWKVD |
| | | | | PEPVTVSWNSGALTSGVHTFPAVLQSSGLYSL | NALQSGNSQESVTEQDSKDSTYSLSSTLTLS |
| | | | | SSVVTVPSSSLGTQTYICNVNHKPSNTKVDKK | KADYEKHKVYACEVTHQGLSSPVTKSFNRGE |
| | | | | VEPKSCDKTHTCPPCPAPELLGGPSVFLFPPK | C |
| | | | | PKDTLMISRTPEVTCVVVDVSHEDPEVKFNWY | |
| | | | | VDGVEVHNAKTKPREEQYNSTYRVVSVLTVL | |
| | | | | HQDWLNGKEYKCKVSNKALPAPIEKTISKAKG | |
| | | | | QPREPQVYTLPPSREEMTKNQVSLTCLVKGF | |
| | | | | YPSDIAVEWESNGQPENNYKTTPPVLDSDGS | |
| | | | | FFLYSKLTVDKSRWQQGNVFSCSVMHEALHN | |
| | | | | HYTQKSLSLSPGK | |
| Endoglin | carotuximab | 653 | 654 | EVKLEESGGGLVQPGGSMKLSCAASGFTFSD | QIVLSQSPAILSASPGEKVTMTCRASSSVSY |
| | | | | AWMDWVRQSPEKGLEWVAEIRSKASNHATY | MHWYQQKPGSSPKPWIYATSNLASGVPVRF |
| | | | | YAESVKGRFTISRDDSKSSVYLQMNSLRAEDT | SGSGSGTSYSLTISRVEAEDAATYYCQQWS |
| | | | | GIYYCTRWRRFFDSWGQGTTLTVSSASTKGP | SNPLTFGAGTKLELKRTVAAPSVFIFPPSDEQ |
| | | | | SVFPLAPSSKSTSGGTAALGCLVKDYFPEPVT | LKSGTASVVCLLNNFYPREAKVQWKVDNALQ |
| | | | | VSWNSGALTSGVHTFPAVLQSSGLYSLSSVVT | SGNSQESVTEQDSKDSTYSLSSTLTLSKADY |
| | | | | VPSSSLGTQTYICNVNHKPSNTKVDKKVEPKS | EKHKVYACEVTHQGLSSPVTKSFNRGEC |
| | | | | CDKTHTCPPCPAPELLGGPSVFLFPPKPKDTL | |
| | | | | MISRTPEVTCVVVDVSHEDPEVKFNWYVDGV | |
| | | | | EVHNAKTKPREEQYNSTYRVVSVLTVLHQDW | |
| | | | | LNGKEYKCKVSNKALPAPIEKTISKAKGQPREP | |
| | | | | QVYTLPPSRDELTKNQVSLTCLVKGFYPSDIA | |
| | | | | VEWESNGQPENNYKTTPPVLDSDGSFFLYSK | |
| | | | | LTVDKSRWQQGNVFSCSVMHEALHNHYTQK | |
| | | | | SLSLSPGK | |
| EpCAM | adecatumumab | 655 | 656 | EVQLLESGGGVVQPGRSLRLSCAASGFTFSS | ELQMTQSPSSLSASVGDRVTITCRTSQSISSY |
| | | | | YGMHWVRQAPGKGLEWVAVISYDGSNKYYA | LNWYQQKPGQPPKLLIYWASTRESGVPDRF |
| | | | | DSVKGRFTISRDNSKNTLYLQMNSLRAEDTAV | SGSGSGTDFTLTISSLQPEDSATYYCQQSYDI |
| | | | | YYCAKDMGWGSGWRPYYYYGMDVWGQGTT | PYTFGQGTKLEIKRTVAAPSVFIFPPSDEQLK |
| | | | | VTVSSASTKGPSVFPLAPSSKSTSGGTAALGC | SGTASVVCLLNNFYPREAKVQWKVDNALQS |
| | | | | LVKDYFPEPVTVSWNSGALTSGVHTFPAVLQS | GNSQESVTEQDSKDSTYSLSSTLTLSKADYE |
| | | | | SGLYSLSSVVTVPSSSLGTQTYICNVNHKPSN | KHKVYACEVTHQGLSSPVTKSFNRGEC |
| | | | | TKVDKKVEPKSCDKTHTCPPCPAPELLGGPSV | |
| | | | | FLFPPKPKDTLMISRTPEVTCVVVDVSHEDPE | |
| | | | | VKFNWYVDGVEVHNAKTKPREEQYNSTYRVV | |
| | | | | SVLTVLHQDWLNGKEYKCKVSNKALPAPIEKTI | |
| | | | | SKAKGQPREPQVYTLPPSRDELTKNQVSLTCL | |
| | | | | VKGFYPSDIAVEWESNGQPENNYKTTPPVLD | |
| | | | | SDGSFFLYSKLTVDKSRWQQGNVFSCSVMHE | |
| | | | | ALHNHYTQKSLSLSPGK | |
| EpCAM | Citatuzumab- | 657 | 658 | HHHHHHEVQLVQSGPGLVQPGGSVRISCAAS | DIQMTQSPSSLSASVGDRVTITCRSTKSLLHS |
| | anti- | | | GYTFTNYGMNWVKQAPGKGLEWMGWINTYT | NGITYLYWYQQKPGKAPKLLIYQMSNLASGV |
| | EpCAM | | | GESTYADSFKGRFTFSLDTSASAAYLQINSLR | PSRFSSSGSGTDFTLTISSLQPEDFATYYCA |
| | FAB fused | | | AEDTAVYYCARFAIKGDYWGQGTLLTVSSAST | QNLEIPRTFGQGTKVELKRTVAAPSVFIFPPS |
| | with rRNA | | | KGPSVFPLAPSSKSTSGGTAALGCLVKDYFPE | DEQLKSGTASVVCLLNNFYPREAKVQWKVD |
| | N- | | | PVTVSWNSGALTSGVHTFPAVLQSSGLYSLSS | NALQSGNSQESVTEQDSKDSTYSLSSTLTLS |
| | glycosidase | | | VVTVPSSSLGTQTYICNVNHKPSNTKVDKKVE | KADYEKHKVYACEVTHQGLSSPVTKSFNRGE |
| | | | | PKSC | CTRHRQPRGWEQLYNTVSFNLGEAYEYPTFI |
| | | | | | QDLRNELAKGTPVCQLPVTLQTIADDKRFVLV |
| | | | | | DITTTSKKTVKVAIDVTDVYVVGYQDKWDGK |
| | | | | | DRAVFLDKVPTVATSKLFPGVTNRVTLTFDGS |
| | | | | | YQKLVNAAKADRKALELGVNKLEFSIEAIHGK |
| | | | | | TINGQEAAKFFLIVIQMVSEAARFKYIETEVVD |
| | | | | | RGLYGSFKPNFKVLNLENNWGDISDAIHKSS |
| | | | | | PQCTTINPALQLISPSNDPWVVNKVSQISPDM |
| | | | | | GILKFKSSK |
| EpCAM | Oportuzumab- | 659 | | HHHHHHDIQMTQSPSSLSASVGDRVTITCRST | |
| | anti- | | | KSLLHSNGITYLYWYQQKPGKAPKLLIYQMS | |
| | EpCAM | | | NLASGVPSRFSSSGSGTDFTLTISSLQPEDFA | |
| | scFv fused | | | TYYCAQNLEIPRTFGQGTKVELKRATPSHNS | |
| | to | | | HQVPSAGGPTANSGTSGSEVQLVQSGPGLV | |
| | Pseudomonas | | | QPGGSVRISCAASGYTFTNYGMNWVKQAPG | |
| | aeruginosa | | | KGLEWMGWINTYTGESTYADSFKGRFTFSLD | |
| | exotoxin A | | | TSASAAYLQINSLRAEDTAVYYCARFAIKGDY | |
| | | | | WGQGTLLTVSSEFGGAPEFPKPSTPPGSSGL | |
| | | | | EGGSLAALTAHQACHLPLETFTRHRQPRGWE | |
| | | | | QLEQCGYPVQRLVALYLAARLSWNQVDQVIR | |
| | | | | NALASPGSGGDLGEAIREQPEQARLALTLAAA | |
| | | | | ESERFVRQGTGNDEAGAASADVVSLTCPVAA | |
| | | | | GECAGPADSGDALLERNYPTGAEFLGDGGDV | |
| | | | | SFSTRGTQNWTVERLLQAHRQLEERGYVFVG | |
| | | | | YHGTFLEAAQSIVFGGVRARSQDLDAIWRGFY | |
| | | | | IAGDPALAYGYAQDQEPDARGRIRNGALLRVY | |
| | | | | VPRSSLPGFYRTGLTLAAPEAAGEVERLIGHP | |
| | | | | LPLRLDAITGPEEEGGRLETILGWPLAERTVVI | |
| | | | | PSAIPTDPRNVGGDLDPSSIPDKEQAISALPDY | |
| | | | | ASQPGKPPHHHHHHKDEL | |
| EpCAM | Solitomab- | 660 | | ELVMTQSPSSLTVTAGEKVTMSCKSSQSLLNS | |
| | bispecific | | | GNQKNYLTWYQQKPGQPPKLLIYWASTRESG | |
| | anti-CD3 | | | VPDRFTGSGSGTDFTLTISSVQAEDLAVYYCQ | |
| | and anti- | | | NDYSYPLTFGAGTKLEIKGGGGSGGGGSGGG | |
| | EPCAM | | | GSEVQLLEQSGAELVRPGTSVKISCKASGYA | |
| | antibody | | | FTNYWLGWVKQRPGHGLEWIGDIFPGSGNIH | |
| | | | | YNEKFKGKATLTADKSSSTAYMQLSSLTFED | |
| | | | | SAVYFCARLRNWDEPMDYWGQGTTVTVSSG | |
| | | | | GGGSDVQLVQSGAEVKKPGASVKVSCKASG | |
| | | | | YTFTRYTMHWVRQAPGQGLEWIGYINPSRGY | |
| | | | | TNYADSVKGRFTITTDKSTSTAYMELSSLRSE | |
| | | | | DTATYYCARYYDDHYCLDYWGQGTTVTVSSG | |
| | | | | EGTSTGSGGSGGSGGADDIVLTQSPATLSLSP | |
| | | | | GERATLSCRASQSVSYMNWYQQKPGKAPKR | |
| | | | | WIYDTSKVASGVPARFSGSGSGTDYSLTINSL | |
| | | | | EAEDAATYYCQQWSSNPLTFGGGTKVEIKHH | |
| | | | | HHHH | |
| EpCAM | Tucotuzumab- | 661 | 662 | EIQLVQSGAEVKKPGETVKISCKASGYTFTNY | EIVLTQSPATLSLSPGERVTLTCSASSSVSYM |
| | anti- | | | GMNWVKQTPGKGLKWMGWINTYTGEPTYAD | LWYQQKPGSSPKPWIFDTSNLASGFPARFS |
| | EpCAM | | | DFKGRFAFSLETSTSTAFLQINNLRSEDTATYE | GSGSGTSYSLIISSMEAEDAATYYCHQRSGY |
| | fused with | | | CVRFISKGDYWGQGTSVTVSSASTKGPSVFP | PYTFGGGTKLEIKRTVAAPSVFIFPPSDEQLK |
| | IL-2 | | | LAPSSKSTSGGTAALGCLVKDYFPEPVTVSW | SGTASVVCLLNNFYPREAKVQWKVDNALQS |
| | | | | NSGALTSGVHTFPAVLQSSGLYSLSSVVTVPS | GNSQESVTEQDSKDSTYSLSSTLTLSKADYE |
| | | | | SSLGTQTYICNVNHKPSNTKVDKRVEPKSCDK | KHKVYACEVTHQGLSSPVTKSFNRGEC |
| | | | | THTCPPCPAPELLGGPSVFLFPPKPKDTLMIS | |
| | | | | RTPEVTCVVVDVSHEDPEVKFNWYVDGVEVH | |
| | | | | NAKTKPREEQYNSTYRVVSVLTVLHQDWLNG | |
| | | | | KEYKCKVSNKALPAPIEKTISKAKGQPREPQV | |
| | | | | YTLPPSREEMTKNQVSLTCLVKGFYPSDIAVE | |
| | | | | WESNGQPENNYKTTPPVLDSDGSFFLYSKLT | |
| | | | | VDKSRWQQGNVFSCSVMHEALHNHYTQKSL | |
| | | | | SLSPGKAPTSSSTKKTQLQLEHLLLDLQMILNG | |
| | | | | INNYKNPKLTRMLTFKFYMPKKATELKHLQCL | |
| | | | | EEELKPLEEVLNLAQSKNFHLRPRDLISNINVIV | |
| | | | | LELKGSETTFMCEYADETATIVEFLNRWITFCQ | |
| | | | | SIISTLT | |
| EPHA3 | ifabotuzumab | 663 | 664 | QVQLVQSGAEVKKPGASVKVSCKASGYTFTG | DIQMTQSPSFLSASVGDRVTITCRASQGIISY |
| | | | | YWMNWVRQAPGQGLEWMGDIYPGSGNTNY | LAWYQQKPEKAPKRLIYAASSLQSGVPSRF |
| | | | | DEKFQGRVTMTRDTSISTAYMELSRLRSDDTA | SGSGSGTEFTLTISSLQPEDFATYYCGQYAN |
| | | | | VYYCARGGYYEDFDSWGQGTTVTVSSASTK | YPYTFGQGTKLEIKRTVAAPSVFIFPPSDEQL |
| | | | | GPSVFPLAPSSKSTSGGTAALGCLVKDYFPEP | KSGTASVVCLLNNFYPREAKVQWKVDNALQ |
| | | | | VTVSWNSGALTSGVHTFPAVLQSSGLYSLSSV | SGNSQESVTEQDSKDSTYSLSSTLTLSKADY |
| | | | | VTVPSSSLGTQTYICNVNHKPSNTKVDKRVEP | EKHKVYACEVTHQGLSSPVTKSFNRGEC |
| | | | | KSCDKTHTCPPCPAPELLGGPSVFLFPPKPKD | |
| | | | | TLMISRTPEVTCVVVDVSHEDPEVKFNWYVD | |
| | | | | GVEVHNAKTKPREEQYNSTYRVVSVLTVLHQ | |
| | | | | DWLNGKEYKCKVSNKALPAPIEKTISKAKGQP | |
| | | | | REPQVYTLPPSREEMTKNQVSLTCLVKGFYPS | |
| | | | | DIAVEWESNGQPENNYKTTPPVLDSDGSFFLY | |
| | | | | SKLTVDKSRWQQGNVFSCSVMHEALHNHYT | |
| | | | | QKSLSLSPGK | |
| EGFR | Amivantamab- | 665 | 666 | QVQLVESGGGVVQPGRSLRLSCAASGFTFST | AIQLTQSPSSLSASVGDRVTITCRASQDISSA |
| | bispecific | | | YGMHWVRQAPGKGLEWVAVIWDDGSYKYYG | LVWYQQKPGKAPKLLIYDASSLESGVPSRFS |
| | anti-EGFR | | | DSVKGRFTISRDNSKNTLYLQMNSLRAEDTAV | GSESGTDFTLTISSLQPEDFATYYCQQFNSY |
| | and anti- | | | YYCARDGITMVRGVMKDYFDYWGQGTLVTVS | PLTFGGGTKVEIKRTVAAPSVFIFPPSDEQLK |
| | HGFR | | | SASTKGPSVFPLAPSSKSTSGGTAALGCLVKD | SGTASVVCLLNNFYPREAKVQWKVDNALQS |
| | antibody | | | YFPEPVTVSWNSGALTSGVHTFPAVLQSSGL | GNSQESVTEQDSKDSTYSLSSTLTLSKADYE |
| | | | | YSLSSVVTVPSSSLGTQTYICNVNHKPSNTKV | KHKVYACEVTHQGLSSPVTKSFNRGEC |
| | | | | DKRVEPKSCDKTHTCPPCPAPELLGGPSVFLF | |
| | | | | PPKPKDTLMISRTPEVTCVVVDVSHEDPEVKF | |
| | | | | NWYVDGVEVHNAKTKPREEQYNSTYRVVSVL | |
| | | | | TVLHQDWLNGKEYKCKVSNKALPAPIEKTISK | |
| | | | | AKGQPREPQVYTLPPSREEMTKNQVSLTCLV | |
| | | | | KGFYPSDIAVEWESNGQPENNYKTTPPVLDS | |
| | | | | DGSFLLYSKLTVDKSRWQQGNVFSCSVMHEA | |
| | | | | LHNHYTQKSLSLSPGK | |
| EGFR | cetuximab | 667 | 668 | QVQLKQSGPGLVQPSQSLSITCTVSGFSLTNY | DILLTQSPVILSVSPGERVSFSCRASQSIGTNI |
| | | | | GVHWVRQSPGKGLEWLGVIWSGGNTDYNTP | HWYQQRTNGSPRLLIKYASESISGIPSRFSG |
| | | | | FTSRLSINKDNSKSQVFFKMNSLQSNDTAIYY | SGSGTDFTLSINSVESEDIADYYCQQNNNWP |
| | | | | CARALTYYDYEFAYWGQGTLVTVSAASTKGP | TTFGAGTKLELKRTVAAPSVFIFPPSDEQLKS |
| | | | | SVFPLAPSSKSTSGGTAALGCLVKDYFPEPVT | GTASVVCLLNNFYPREAKVQWKVDNALQSG |
| | | | | VSWNSGALTSGVHTFPAVLQSSGLYSLSSVVT | NSQESVTEQDSKDSTYSLSSTLTLSKADYEK |
| | | | | VPSSSLGTQTYICNVNHKPSNTKVDKRVEPKS | HKVYACEVTHQGLSSPVTKSFNRGEC |
| | | | | CDKTHTCPPCPAPELLGGPSVFLFPPKPKDTL | |
| | | | | MISRTPEVTCVVVDVSHEDPEVKFNWYVDGV | |
| | | | | EVHNAKTKPREEQYNSTYRVVSVLTVLHQDW | |
| | | | | LNGKEYKCKVSNKALPAPIEKTISKAKGQPREP | |
| | | | | QVYTLPPSREEMTKNQVSLTCLVKGFYPSDIA | |
| | | | | VEWESNGQPENNYKTTPPVLDSDGSFFLYSK | |
| | | | | LTVDKSRWQQGNVFSCSVMHEALHNHYTQK | |
| | | | | SLSLSPGK | |
| EGFR | depatuxizumab | 669 | 670 | QVQLQESGPGLVKPSQTLSLTCTVSGYSISSD | DIQMTQSPSSMSVSVGDRVTITCHSSQDINS |
| | | | | FAWNWIRQPPGKGLEWMGYISYSGNTRYQP | NIGWLQQKPGKSFKGLIYHGTNLDDGVPSRF |
| | | | | SLKSRITISRDTSKNQFFLKLNSVTAADTATYY | SGSGSGTDYTLTISSLQPEDFATYYCVQYAQ |
| | | | | CVTAGRGFPYWGQGTLVTVSSASTKGPSVFP | FPWTFGGGTKLEIKRTVAAPSVFIFPPSDEQL |
| | | | | LAPSSKSTSGGTAALGCLVKDYFPEPVTVSW | KSGTASVVCLLNNFYPREAKVQWKVDNALQ |
| | | | | NSGALTSGVHTFPAVLQSSGLYSLSSVVTVPS | SGNSQESVTEQDSKDSTYSLSSTLTLSKADY |
| | | | | SSLGTQTYICNVNHKPSNTKVDKKVEPKSCDK | EKHKVYACEVTHQGLSSPVTKSFNRGEC |
| | | | | THTCPPCPAPELLGGPSVFLFPPKPKDTLMIS | |
| | | | | RTPEVTCVVVDVSHEDPEVKFNWYVDGVEVH | |
| | | | | NAKTKPREEQYNSTYRVVSVLTVLHQDWLNG | |
| | | | | KEYKCKVSNKALPAPIEKTISKAKGQPREPQV | |
| | | | | YTLPPSRDELTKNQVSLTCLVKGFYPSDIAVE | |
| | | | | WESNGQPENNYKTTPPVLDSDGSFFLYSKLT | |
| | | | | VDKSRWQQGNVFSCSVMHEALHNHYTQKSL | |
| | | | | SLSPGK | |
| EGFR | futuximab | 671 | 672 | EVQLQQPGSELVRPGASVKLSCKASGYTFTS | DIQMTQTTSSLSASLGDRVTISCRTSQDIGNY |
| | | | | YWMHWVKQRPGQGLEWIGNIYPGSRSTNYD | LNWYQQKPDGTVKLLIYYTSRLHSGVPSRFS |
| | | | | EKFKSKATLTVDTSSSTAYMQLSSLTSEDSAV | GSGSGTDFSLTINNVEQEDVATYFCQHYNTV |
| | | | | YYCTRNGDYYVSSGDAMDYWGQGTSVTVSS | PPTFGGGTKLEIKRTVAAPSVFIFPPSDEQLK |
| | | | | ASTKGPSVFPLAPSSKSTSGGTAALGCLVKDY | SGTASVVCLLNNFYPREAKVQWKVDNALQS |
| | | | | FPEPVTVSWNSGALTSGVHTFPAVLQSSGLY | GNSQESVTEQDSKDSTYSLSSTLTLSKADYE |
| | | | | SLSSVVTVPSSSLGTQTYICNVNHKPSNTKVD | KHKVYACEVTHQGLSSPVTKSFNRGEC |
| | | | | KRVEPKSCDKTHTCPPCPAPELLGGPSVFLFP | |
| | | | | PKPKDTLMISRTPEVTCVVVDVSHEDPEVKFN | |
| | | | | WYVDGVEVHNAKTKPREEQYNSTYRVVSVLT | |
| | | | | VLHQDWLNGKEYKCKVSNKALPAPIEKTISKA | |
| | | | | KGQPREPQVYTLPPSREEMTKNQVSLTCLVK | |
| | | | | GFYPSDIAVEWESNGQPENNYKTTPPVLDSD | |
| | | | | GSFFLYSKLTVDKSRWQQGNVFSCSVMHEAL | |
| | | | | HNHYTQKSLSLSPG | |
| EGFR | imgatuzumab | 673 | 674 | QVQLVQSGAEVKKPGSSVKVSCKASGFTFTD | DIQMTQSPSSLSASVGDRVTITCRASQGINN |
| | | | | YKIHWVRQAPGQGLEWMGYFNPNSGYSTYA | YLNWYQQKPGKAPKRLIYNTNNLQTGVPSR |
| | | | | QKFQGRVTITADKSTSTAYMELSSLRSEDTAV | FSGSGSGTEFTLTISSLQPEDFATYYCLQHN |
| | | | | YYCARLSPGGYYVMDAWGQGTTVTVSSASTK | SFPTFGQGTKLEIKRTVAAPSVFIFPPSDEQL |
| | | | | GPSVFPLAPSSKSTSGGTAALGCLVKDYFPEP | KSGTASVVCLLNNFYPREAKVQWKVDNALQ |
| | | | | VTVSWNSGALTSGVHTFPAVLQSSGLYSLSSV | SGNSQESVTEQDSKDSTYSLSSTLTLSKADY |
| | | | | VTVPSSSLGTQTYICNVNHKPSNTKVDKKVEP | EKHKVYACEVTHQGLSSPVTKSFNRGEC |
| | | | | KSCDKTHTCPPCPAPELLGGPSVFLFPPKPKD | |
| | | | | TLMISRTPEVTCVVVDVSHEDPEVKFNWYVD | |
| | | | | GVEVHNAKTKPREEQYNSTYRVVSVLTVLHQ | |
| | | | | DWLNGKEYKCKVSNKALPAPIEKTISKAKGQP | |
| | | | | REPQVYTLPPSRDELTKNQVSLTCLVKGFYPS | |
| | | | | DIAVEWESNGQPENNYKTTPPVLDSDGSFFLY | |
| | | | | SKLTVDKSRWQQGNVFSCSVMHEALHNHYT | |
| | | | | QKSLSLSPG | |
| EGFR | laprituximab | 675 | 676 | QVQLVQSGAEVAKPGASVKLSCKASGYTFTS | DIQMTQSPSSLSASVGDRVTITCRASQDINNY |
| | | | | YWMQWVKQRPGQGLECIGTIYPGDGDTTYTQ | LAWYQHKPGKGPKLLIHYTSTLHPGIPSRFS |
| | | | | KFQGKATLTADKSSSTAYMQLSSLRSEDSAVY | GSGSGRDYSFSISSLEPEDIATYYCLQYDNLL |
| | | | | YCARYDAPGYAMDYWGQGTLVTVSSASTKG | YTFGQGTKLEIKRTVAAPSVFIFPPSDEQLKS |
| | | | | PSVFPLAPSSKSTSGGTAALGCLVKDYFPEPV | GTASVVCLLNNFYPREAKVQWKVDNALQSG |
| | | | | TVSWNSGALTSGVHTFPAVLQSSGLYSLSSVV | NSQESVTEQDSKDSTYSLSSTLTLSKADYEK |
| | | | | TVPSSSLGTQTYICNVNHKPSNTKVDKKVEPK | HKVYACEVTHQGLSSPVTKSFNRGEC |
| | | | | SCDKTHTCPPCPAPELLGGPSVFLFPPKPKDT | |
| | | | | LMISRTPEVTCVVVDVSHEDPEVKFNWYVDG | |
| | | | | VEVHNAKTKPREEQYNSTYRVVSVLTVLHQD | |
| | | | | WLNGKEYKCKVSNKALPAPIEKTISKAKGQPR | |
| | | | | EPQVYTLPPSRDELTKNQVSLTCLVKGFYPSD | |
| | | | | IAVEWESNGQPENNYKTTPPVLDSDGSFFLYS | |
| | | | | KLTVDKSRWQQGNVFSCSVMHEALHNHYTQ | |
| | | | | KSLSLSPG | |
| EGFR | losatuxizumab | 677 | 678 | EVQLQESGPGLVKPSQTLSLTCTVSGYSISRD | DIQMTQSPSSMSVSVGDRVTITCHSSQDINS |
| | | | | FAWNWIRQPPGKGLEWMGYISYNGNTRYQP | NIGWLQQKPGKSFKGLIYHGTNLDDGVPSRF |
| | | | | SLKSRITISRDTSKNQFFLKLNSVTAADTATYY | SGSGSGTDYTLTISSLQPEDFATYYCVQYAQ |
| | | | | CVTASRGFPYWGQGTLVTVSSASTKGPSVFP | FPWTFGGGTKLEIKRTVAAPSVFIFPPSDEQL |
| | | | | LAPSSKSTSGGTAALGCLVKDYFPEPVTVSW | KSGTASVVCLLNNFYPREAKVQWKVDNALQ |
| | | | | NSGALTSGVHTFPAVLQSSGLYSLSSVVTVPS | SGNSQESVTEQDSKDSTYSLSSTLTLSKADY |
| | | | | SSLGTQTYICNVNHKPSNTKVDKKVEPKSCDK | EKHKVYACEVTHQGLSSPVTKSFNRGEC |
| | | | | THTCPPCPAPELLGGPSVFLFPPKPKDTLMIS | |
| | | | | RTPEVTCVVVDVSHEDPEVKFNWYVDGVEVH | |
| | | | | NAKTKPREEQYNSTYRVVSVLTVLHQDWLNG | |
| | | | | KEYKCKVSNKALPAPIEKTISKAKGQPREPQV | |
| | | | | YTLPPSREEMTKNQVSLTCLVKGFYPSDIAVE | |
| | | | | WESNGQPENNYKTTPPVLDSDGSFFLYSKLT | |
| | | | | VDKSRWQQGNVFSCSVMHEALHNHYTQKSL | |
| | | | | SLSPGK | |
| EGFR | Matuzumab- | 853 | 854 | QVQLVQSGAEVKKPGASVKVSCKASGYTFTS | DIQMTQSPSSLSASVGDRVTITCSASSSVTY |
| | EGFR | | | HWMHWVRQAPGQGLEWIGEFNPSNGRTNYN | MYWYQQKPGKAPKLLIYDTSNLASGVPSRF |
| | binding | | | EKFKSKATMTVDTSTNTAYMELSSLRSEDTAV | SGSGSGTDYTFTISSLQPEDIATYYCQQWSS |
| | fragment | | | YYCASRDYDYAGRYFDYWGQGTLVTVSSA | HIFTFGQGTKVEIKR |
| EGFR | modotuximab | 679 | 680 | QVQLQQPGAELVEPGGSVKLSCKASGYTFTS | DIVMTQAAFSNPVTLGTSASISCRSSKSLLHS |
| | | | | HWMHWVKQRPGQGLEWIGEINPSSGRNNYN | NGITYLYWYLQKPGQSPQLLIYQMSNLASGV |
| | | | | EKFKSKATLTVDKSSSTAYMQFSSLTSEDSAV | PDRFSSSGSGTDFTLRISRVEAEDVGVYYCA |
| | | | | YYCVRYYGYDEAMDYWGQGTSVTVSSASTK | QNLELPYTFGGGTKLEIKRTVAAPSVFIFPPS |
| | | | | GPSVFPLAPSSKSTSGGTAALGCLVKDYFPEP | DEQLKSGTASVVCLLNNFYPREAKVQWKVD |
| | | | | VTVSWNSGALTSGVHTFPAVLQSSGLYSLSSV | NALQSGNSQESVTEQDSKDSTYSLSSTLTLS |
| | | | | VTVPSSSLGTQTYICNVNHKPSNTKVDKRVEP | KADYEKHKVYACEVTHQGLSSPVTKSFNRGE |
| | | | | KSCDKTHTCPPCPAPELLGGPSVFLFPPKPKD | C |
| | | | | TLMISRTPEVTCVVVDVSHEDPEVKFNWYVD | |
| | | | | GVEVHNAKTKPREEQYNSTYRVVSVLTVLHQ | |
| | | | | DWLNGKEYKCKVSNKALPAPIEKTISKAKGQP | |
| | | | | REPQVYTLPPSREEMTKNQVSLTCLVKGFYPS | |
| | | | | DIAVEWESNGQPENNYKTTPPVLDSDGSFFLY | |
| | | | | SKLTVDKSRWQQGNVFSCSVMHEALHNHYT | |
| | | | | QKSLSLSPG | |
| EGFR | necitumumab | 681 | 682 | QVQLQESGPGLVKPSQTLSLTCTVSGGSISSG | EIVMTQSPATLSLSPGERATLSCRASQSVSS |
| | | | | DYYWSWIRQPPGKGLEWIGYIYYSGSTDYNP | YLAWYQQKPGQAPRLLIYDASNRATGIPARF |
| | | | | SLKSRVTMSVDTSKNQFSLKVNSVTAADTAVY | SGSGSGTDFTLTISSLEPEDFAVYYCHQYGS |
| | | | | YCARVSIFGVGTFDYWGQGTLVTVSSASTKG | TPLTFGGGTKAEIKRTVAAPSVFIFPPSDEQL |
| | | | | PSVLPLAPSSKSTSGGTAALGCLVKDYFPEPV | KSGTASVVCLLNNFYPREAKVQWKVDNALQ |
| | | | | TVSWNSGALTSGVHTFPAVLQSSGLYSLSSVV | SGNSQESVTEQDSKDSTYSLSSTLTLSKADY |
| | | | | TVPSSSLGTQTYICNVNHKPSNTKVDKRVEPK | EKHKVYACEVTHQGLSSPVTKSFNRGEC |
| | | | | SCDKTHTCPPCPAPELLGGPSVFLFPPKPKDT | |
| | | | | LMISRTPEVTCVVVDVSHEDPEVKFNWYVDG | |
| | | | | VEVHNAKTKPREEQYNSTYRVVSVLTVLHQD | |
| | | | | WLNGKEYKCKVSNKALPAPIEKTISKAKGQPR | |
| | | | | EPQVYTLPPSREEMTKNQVSLTCLVKGFYPSD | |
| | | | | IAVEWESNGQPENNYKTTPPVLDSDGSFFLYS | |
| | | | | KLTVDKSRWQQGNVFSCSVMHEALHNHYTQ | |
| | | | | KSLSLSPGK | |
| EGFR | nimotuzumab | 683 | 684 | QVQLQQSGAEVKKPGSSVKVSCKASGYTFTN | DIQMTQSPSSLSASVGDRVTITCRSSQNIVHS |
| | | | | YYIYWVRQAPGQGLEWIGGINPTSGGSNFNE | NGNTYLDWYQQTPGKAPKLLIYKVSNRFSG |
| | | | | KFKTRVTITVDESTNTAYMELSSLRSEDTAFYF | VPSRFSGSGSGTDFTFTISSLQPEDIATYYCF |
| | | | | CARQGLWFDSDGRGFDFWGQGSTVTVSSAS | QYSHVPWTFGQGTKLQITRTVAAPSVFIFPPS |
| | | | | TKGPSVFPLAPSSKSTSGGTAALGCLVKDYFP | DEQLKSGTASVVCLLNNFYPREAKVQWKVD |
| | | | | EPVTVSWNSGALTSGVHTFPAVLQSSGLYSLS | NALQSGNSQESVTEQDSKDSTYSLSSTLTLS |
| | | | | SVVTVPSSSLGTQTYICNVNHKPSNTKVDKKV | KADYEKHKVYACEVTHQGLSSPVTKSFNRGE |
| | | | | EPKSCDKTHTCPPCPAPELLGGPSVFLFPPKP | C |
| | | | | KDTLMISRTPEVTCVVVDVSHEDPEVKFNWYV | |
| | | | | DGVEVHNAKTKPREEQYNSTYRVVSVLTVLH | |
| | | | | QDWLNGKEYKCKVSNKALPAPIEKTISKAKGQ | |
| | | | | PREPQVYTLPPSRDELTKNQVSLTCLVKGFYP | |
| | | | | SDIAVEWESNGQPENNYKTTPPVLDSDGSFFL | |
| | | | | YSKLTVDKSRWQQGNVFSCSVMHEALHNHYT | |
| | | | | QKSLSLSPGK | |
| EGFR | panitumumab | 685 | 686 | QVQLQESGPGLVKPSETLSLTCTVSGGSVSS | DIQMTQSPSSLSASVGDRVTITCQASQDISNY |
| | | | | GDYYWTWIRQSPGKGLEWIGHIYYSGNTNYN | LNWYQQKPGKAPKLLIYDASNLETGVPSRF |
| | | | | PSLKSRLTISIDTSKTQFSLKLSSVTAADTAIYY | SGSGSGTDFTFTISSLQPEDIATYFCQHFDHL |
| | | | | CVRDRVTGAFDIWGQGTMVTVSSASTKGPSV | PLAFGGGTKVEIKRTVAAPSVFIFPPSDEQLK |
| | | | | FPLAPCSRSTSESTAALGCLVKDYFPEPVTVS | SGTASVVCLLNNFYPREAKVQWKVDNALQS |
| | | | | WNSGALTSGVHTFPAVLQSSGLYSLSSVVTVP | GNSQESVTEQDSKDSTYSLSSTLTLSKADYE |
| | | | | SSNFGTQTYTCNVDHKPSNTKVDKTVERKCC | KHKVYACEVTHQGLSSPVTKSFNRGEC |
| | | | | VECPPCPAPPVAGPSVFLFPPKPKDTLMISRT | |
| | | | | PEVTCVVVDVSHEDPEVQFNWYVDGVEVHNA | |
| | | | | KTKPREEQFNSTFRVVSVLTVVHQDWLNGKE | |
| | | | | YKCKVSNKGLPAPIEKTISKTKGQPREPQVYTL | |
| | | | | PPSREEMTKNQVSLTCLVKGFYPSDIAVEWES | |
| | | | | NGQPENNYKTTPPMLDSDGSFFLYSKLTVDK | |
| | | | | SRWQQGNVFSCSVMHEALHNHYTQKSLSLSP | |
| | | | | GK | |
| EGFR | tomuzotuximab | 687 | 688 | QVQLKQSGPGLVQPSQSLSITCTVSGFSLTNY | DILLTQSPVILSVSPGERVSFSCRASQSIGTNI |
| | | | | GVHWVRQSPGKGLEWLGVIWSGGNTDYNTP | HWYQQRTNGSPRLLIKYASESISGIPSRFSG |
| | | | | FTSRLSINKDNSKSQVFFKMNSLQSNDTAIYY | SGSGTDFTLSINSVESEDIADYYCQQNNNWP |
| | | | | CARALTYYDYEFAYWGQGTLVTVSTASTKGP | TTFGAGTKLELKRTVAAPSVFIFPPSDEQLKS |
| | | | | SVFPLAPSSKSTSGGTAALGCLVKDYFPEPVT | GTASVVCLLNNFYPREAKVQWKVDNALQSG |
| | | | | VSWNSGALTSGVHTFPAVLQSSGLYSLSSVVT | NSQESVTEQDSKDSTYSLSSTLTLSKADYEK |
| | | | | VPSSSLGTQTYICNVNHKPSNTKVDKKVEPKS | HKVYACEVTHQGLSSPVTKSFNRGEC |
| | | | | CDKTHTCPPCPAPELLGGPSVFLFPPKPKDTL | |
| | | | | MISRTPEVTCVVVDVSHEDPEVKFNWYVDGV | |
| | | | | EVHNAKTKPREEQYNSTYRVVSVLTVLHQDW | |
| | | | | LNGKEYKCKVSNKALPAPIEKTISKAKGQPREP | |
| | | | | QVYTLPPSRDELTKNQVSLTCLVKGFYPSDIA | |
| | | | | VEWESNGQPENNYKTTPPVLDSDGSFFLYSK | |
| | | | | LTVDKSRWQQGNVFSCSVMHEGLHNHYTQK | |
| | | | | SLSLSPGK | |
| EGFR | zalutumumab | 689 | 690 | QVQLVESGGGVVQPGRSLRLSCAASGFTFST | AIQLTQSPSSLSASVGDRVTITCRASQDISSA |
| | | | | YGMHWVRQAPGKGLEWVAVIWDDGSYKYYG | LVWYQQKPGKAPKLLIYDASSLESGVPSRFS |
| | | | | DSVKGRFTISRDNSKNTLYLQMNSLRAEDTAV | GSESGTDFTLTISSLQPEDFATYYCQQFNSY |
| | | | | YYCARDGITMVRGVMKDYFDYWGQGTLVTVS | PLTFGGGTKVEIKRTVAAPSVFIFPPSDEQLK |
| | | | | SASTKGPSVFPLAPSSKSTSGGTAALGCLVKD | SGTASVVCLLNNFYPREAKVQWKVDNALQS |
| | | | | YFPEPVTVSWNSGALTSGVHTFPAVLQSSGL | GNSQESVTEQDSKDSTYSLSSTLTLSKADYE |
| | | | | YSLSSVVTVPSSSLGTQTYICNVNHKPSNTKV | KHKVYACEVTHQGLSSPVTKSFNRGEC |
| | | | | DKRVEPKSCDKTHTCPPCPAPELLGGPSVFLF | |
| | | | | PPKPKDTLMISRTPEVTCVVVDVSHEDPEVKF | |
| | | | | NWYVDGVEVHNAKTKPREEQYNSTYRVVSVL | |
| | | | | TVLHQDWLNGKEYKCKVSNKALPAPIEKTISK | |
| | | | | AKGQPREPQVYTLPPSREEMTKNQVSLTCLV | |
| | | | | KGFYPSDIAVEWESNGQPENNYKTTPPVLDS | |
| | | | | DGSFFLYSKLTVDKSRWQQGNVFSCSVMHEA | |
| | | | | LHNHYTQKSLSLSPGK | |
| FGFR2 | bemarituzumab | 691 | 692 | QVQLVQSGAEVKKPGSSVKVSCKASGYIFTTY | DIQMTQSPSSLSASVGDRVTITCKASQGVSN |
| | | | | NVHWVRQAPGQGLEWIGSIYPDNGDTSYNQN | DVAWYQQKPGKAPKLLIYSASYRYTGVPSR |
| | | | | FKGRATITADKSTSTAYMELSSLRSEDTAVYY | FSGSGSGTDFTFTISSLQPEDIATYYCQQHST |
| | | | | CARGDFAYWGQGTLVTVSSASTKGPSVFPLA | TPYTFGQGTKLEIKRTVAAPSVFIFPPSDEQL |
| | | | | PSSKSTSGGTAALGCLVKDYFPEPVTVSWNS | KSGTASVVCLLNNFYPREAKVQWKVDNALQ |
| | | | | GALTSGVHTFPAVLQSSGLYSLSSVVTVPSSS | SGNSQESVTEQDSKDSTYSLSSTLTLSKADY |
| | | | | LGTQTYICNVNHKPSNTKVDKRVEPKSCDKTH | EKHKVYACEVTHQGLSSPVTKSFNRGEC |
| | | | | TCPPCPAPELLGGPSVFLFPPKPKDTLMISRTP | |
| | | | | EVTCVVVDVSHEDPEVKFNWYVDGVEVHNAK | |
| | | | | TKPREEQYNSTYRVVSVLTVLHQDWLNGKEY | |
| | | | | KCKVSNKALPAPIEKTISKAKGQPREPQVYTLP | |
| | | | | PSREEMTKNQVSLTCLVKGFYPSDIAVEWESN | |
| | | | | GQPENNYKTTPPVLDSDGSFFLYSKLTVDKSR | |
| | | | | WQQGNVFSCSVMHEALHNHYTQKSLSLSPG | |
| | | | | K | |
| Fibronectin | radretumab | 693 | | EVQLLESGGGLVQPGGSLRLSCAASGFTFSS | |
| extra | scFv- | | | FSMSWVRQAPGKGLEWVSSISGSSGTTYYAD | |
| domain-B | protein | | | SVKGRFTISRDNSKNTLYLQMNSLRAEDTAVY | |
| | fusion | | | YCAKPFPYFDYWGQGTLVTVSSGDGSSGGS | |
| | | | | GGASEIVLTQSPGTLSLSPGERATLSCRASQS | |
| | | | | VSSSFLAWYQQKPGQAPRLLIYYASSRATGIP | |
| | | | | DRFSGSGSGTDFTLTISRLEPEDFAVYYCQQT | |
| | | | | GRIPPTFGQGTKVEIKSGGSGGPRAAPEVYAF | |
| | | | | ATPEWPGSRDKRTLACLIQNFMPEDISVQWLH | |
| | | | | NEVQLPDARHSTTQPRKTKGSGFFVFSRLEVT | |
| | | | | RAEWEQKDEFICRAVHEAASPSQTVQRAVSV | |
| | | | | NPESSRRGGC | |
| Folate | farletuzumab | 694 | 695 | EVQLVESGGGVVQPGRSLRLSCSASGFTFSG | DIQLTQSPSSLSASVGDRVTITCSVSSSISSN |
| receptor 1 | | | | YGLSWVRQAPGKGLEWVAMISSGGSYTYYAD | NLHWYQQKPGKAPKPWIYGTSNLASGVPSR |
| | | | | SVKGRFAISRDNAKNTLFLQMDSLRPEDTGVY | FSGSGSGTDYTFTISSLQPEDIATYYCQQWS |
| | | | | FCARHGDDPAWFAYWGQGTPVTVSSASTKG | SYPYMYTFGQGTKVEIKRTVAAPSVFIFPPSD |
| | | | | PSVFPLAPSSKSTSGGTAALGCLVKDYFPEPV | EQLKSGTASVVCLLNNFYPREAKVQWKVDN |
| | | | | TVSWNSGALTSGVHTFPAVLQSSGLYSLSSVV | ALQSGNSQESVTEQDSKDSTYSLSSTLTLSK |
| | | | | TVPSSSLGTQTYICNVNHKPSNTKVDKKVEPK | ADYEKHKVYACEVTHQGLSSPVTKSFNRGEC |
| | | | | SCDKTHTCPPCPAPELLGGPSVFLFPPKPKDT | |
| | | | | LMISRTPEVTCVVVDVSHEDPEVKFNWYVDG | |
| | | | | VEVHNAKTKPREEQYNSTYRVVSVLTVLHQD | |
| | | | | WLNGKEYKCKVSNKALPAPIEKTISKAKGQPR | |
| | | | | EPQVYTLPPSRDELTKNQVSLTCLVKGFYPSD | |
| | | | | IAVEWESNGQPENNYKTTPPVLDSDGSFFLYS | |
| | | | | KLTVDKSRWQQGNVFSCSVMHEALHNHYTQ | |
| | | | | KSLSLSPGK | |
| Folate | mirvetuximab | 696 | 697 | QVQLVQSGAEVVKPGASVKISCKASGYTFTGY | DIVLTQSPLSLAVSLGQPAIISCKASQSVSFA |
| receptor 1 | | | | FMNWVKQSPGQSLEWIGRIHPYDGDTFYNQK | GTSLMHWYHQKPGQQPRLLIYRASNLEAGV |
| | | | | FQGKATLTVDKSSNTAHMELLSLTSEDFAVYY | PDRFSGSGSKTDFTLTISPVEAEDAATYYCQ |
| | | | | CTRYDGSRAMDYWGQGTTVTVSSASTKGPS | QSREYPYTFGGGTKLEIKRTVAAPSVFIFPPS |
| | | | | VFPLAPSSKSTSGGTAALGCLVKDYFPEPVTV | DEQLKSGTASVVCLLNNFYPREAKVQWKVD |
| | | | | SWNSGALTSGVHTFPAVLQSSGLYSLSSVVTV | NALQSGNSQESVTEQDSKDSTYSLSSTLTLS |
| | | | | PSSSLGTQTYICNVNHKPSNTKVDKKVEPKSC | KADYEKHKVYACEVTHQGLSSPVTKSFNRGE |
| | | | | DKTHTCPPCPAPELLGGPSVFLFPPKPKDTLM | C |
| | | | | ISRTPEVTCVVVDVSHEDPEVKFNWYVDGVE | |
| | | | | VHNAKTKPREEQYNSTYRVVSVLTVLHQDWL | |
| | | | | NGKEYKCKVSNKALPAPIEKTISKAKGQPREP | |
| | | | | QVYTLPPSRDELTKNQVSLTCLVKGFYPSDIA | |
| | | | | VEWESNGQPENNYKTTPPVLDSDGSFFLYSK | |
| | | | | LTVDKSRWQQGNVFSCSVMHEALHNHYTQK | |
| | | | | SLSLSPG | |
| FZD1 | vantictumab | 698 | 699 | EVQLVESGGGLVQPGGSLRLSCAASGFTFSH | DIELTQPPSVSVAPGQTARISCSGDNIGSFYV |
| | | | | YTLSWVRQAPGKGLEWVSVISGDGSYTYYAD | HWYQQKPGQAPVLVIYDKSNRPSGIPERFS |
| | | | | SVKGRFTISSDNSKNTLYLQMNSLRAEDTAVY | GSNSGNTATLTISGTQAEDEADYYCQSYANT |
| | | | | YCARNFIKYVFANWGQGTLVTVSSASTKGPSV | LSLVFGGGTKLTVLGQPKAAPSVTLFPPSSE |
| | | | | FPLAPCSRSTSESTAALGCLVKDYFPEPVTVS | ELQANKATLVCLISDFYPGAVTVAWKADSSP |
| | | | | WNSGALTSGVHTFPAVLQSSGLYSLSSVVTVP | VKAGVETTTPSKQSNNKYAASSYLSLTPEQW |
| | | | | SSNFGTQTYTCNVDHKPSNTKVDKTVERKCC | KSHRSYSCQVTHEGSTVEKTVAPTECS |
| | | | | VECPPCPAPPVAGPSVFLFPPKPKDTLMISRT | |
| | | | | PEVTCVVVDVSHEDPEVQFNWYVDGVEVHNA | |
| | | | | KTKPREEQFNSTFRVVSVLTVVHQDWLNGKE | |
| | | | | YKCKVSNKGLPAPIEKTISKTKGQPREPQVYTL | |
| | | | | PPSREEMTKNQVSLTCLVKGFYPSDIAVEWES | |
| | | | | NGQPENNYKTTPPMLDSDGSFFLYSKLTVDK | |
| | | | | SRWQQGNVFSCSVMHEALHNHYTQKSLSLSP | |
| | | | | G | |
| GPRC5D | talquetamab | 700 | 701 | QVQLVQSGAEVKKPGASVKVSCKASGYSFTG | DIQMTQSPSSLSASVGDRVTITCKASQNVAT |
| | | | | YTMNWVRQAPGQGLEWMGLINPYNSDTNYA | HVGWYQQKPGKAPKRLIYSASYRYSGVPSR |
| | | | | QKLQGRVTMTTDTSTSTAYMELRSLRSDDTA | FSGSGSGTEFTLTISNLQPEDFATYYCQQYN |
| | | | | VYYCARVALRVALDYWGQGTLVTVSSASTKG | RYPYTFGQGTKLEIKRTVAAPSVFIFPPSDEQ |
| | | | | PSVFPLAPCSRSTSESTAALGCLVKDYFPEPV | LKSGTASVVCLLNNFYPREAKVQWKVDNALQ |
| | | | | TVSWNSGALTSGVHTFPAVLQSSGLYSLSSVV | SGNSQESVTEQDSKDSTYSLSSTLTLSKADY |
| | | | | TVPSSSLGTKTYTCNVDHKPSNTKVDKRVESK | EKHKVYACEVTHQGLSSPVTKSFNRGEC |
| | | | | YGPPCPPCPAPEAAGGPSVFLFPPKPKDTLMI | |
| | | | | SRTPEVTCVVVDVSQEDPEVQFNWYVDGVEV | |
| | | | | HNAKTKPREEQFNSTYRVVSVLTVLHQDWLN | |
| | | | | GKEYKCKVSNKGLPSSIEKTISKAKGQPREPQ | |
| | | | | VYTLPPSQEEMTKNQVSLTCLVKGFYPSDIAV | |
| | | | | EWESNGQPENNYKTTPPVLDSDGSFFLYSRL | |
| | | | | TVDKSRWQEGNVFSCSVMHEALHNHYTQKSL | |
| | | | | SLSLGK | |
| GD2 | dinutuximab | 702 | 703 | EVQLLQSGPELEKPGASVMISCKASGSSFTGY | EIVMTQSPATLSVSPGERATLSCRSSQSLVH |
| | | | | NMNWVRQNIGKSLEWIGAIDPYYGGTSYNQK | RNGNTYLHWYLQKPGQSPKLLIHKVSNRFS |
| | | | | FKGRATLTVDKSSSTAYMHLKSLTSEDSAVYY | GVPDRFSGSGSGTDFTLKISRVEAEDLGVYF |
| | | | | CVSGMEYWGQGTSVTVSSASTKGPSVFPLAP | CSQSTHVPPLTFGAGTKLELKRTVAAPSVFIF |
| | | | | SSKSTSGGTAALGCLVKDYFPEPVTVSWNSG | PPSDEQLKSGTASVVCLLNNFYPREAKVQWK |
| | | | | ALTSGVHTFPAVLQSSGLYSLSSVVTVPSSSL | VDNALQSGNSQESVTEQDSKDSTYSLSSTLT |
| | | | | GTQTYICNVNHKPSNTKVDKRVEPKSCDKTHT | LSKADYEKHKVYACEVTHQGLSSPVTKSFNR |
| | | | | CPPCPAPELLGGPSVFLFPPKPKDTLMISRTP | GEC |
| | | | | EVTCVVVDVSHEDPEVKFNWYVDGVEVHNAK | |
| | | | | TKPREEQYNSTYRVVSVLTVLHQDWLNGKEY | |
| | | | | KCKVSNKALPAPIEKTISKAKGQPREPQVYTLP | |
| | | | | PSREEMTKNQVSLTCLVKGFYPSDIAVEWESN | |
| | | | | GQPENNYKTTPPVLDSDGSFFLYSKLTVDKSR | |
| | | | | WQQGNVFSCSVMHEALHNHYTQKSLSLSPG | |
| | | | | K | |
| GD2 | naxitamab | 704 | 705 | QVQLVESGPGVVQPGRSLRISCAVSGFSVTN | EIVMTQTPATLSVSAGERVTITCKASQSVSN |
| | | | | YGVHWVRQPPGKGLEWLGVIWAGGITNYNSA | DVTWYQQKPGQAPRLLIYSASNRYSGVPAR |
| | | | | FMSRLTISKDNSKNTVYLQMNSLRAEDTAMYY | FSGSGYGTEFTFTISSVQSEDFAVYFCQQDY |
| | | | | CASRGGHYGYALDYWGQGTLVTVSSASTKG | SSFGQGTKLEIKRTVAAPSVFIFPPSDEQLKS |
| | | | | PSVFPLAPSSKSTSGGTAALGCLVKDYFPEPV | GTASVVCLLNNFYPREAKVQWKVDNALQSG |
| | | | | TVSWNSGALTSGVHTFPAVLQSSGLYSLSSVV | NSQESVTEQDSKDSTYSLSSTLTLSKADYEK |
| | | | | TVPSSSLGTQTYICNVNHKPSNTKVDKRVEPK | HKVYACEVTHQGLSSPVTKSFNRGEC |
| | | | | SCDKTHTCPPCPAPELLGGPSVFLFPPKPKDT | |
| | | | | LMISRTPEVTCVVVDVSHEDPEVKFNWYVDG | |
| | | | | VEVHNAKTKPREEQYNSTYRVVSVLTVLHQD | |
| | | | | WLNGKEYKCKVSNKALPAPIEKTISKAKGQPR | |
| | | | | EPQVYTLPPSRDELTKNQVSLTCLVKGFYPSD | |
| | | | | IAVEWESNGQPENNYKTTPPVLDSDGSFFLYS | |
| | | | | KLTVDKSRWQQGNVFSCSVMHEALHNHYTQ | |
| | | | | KSLSLSPGK | |
| GD3 | ecromeximab | 706 | 707 | EVTLVESGGDFVKPGGSLKVSCAASGFAFSH | DIQMTQTASSLPASLGDRVTISCSASQDISNY |
| | | | | YAMSWVRQTPAKRLEWVAYISSGGSGTYYSD | LNWYQQKPDGTVKLLIFYSSNLHSGVPSRFS |
| | | | | SVKGRFTISRDNAKNTLYLQMRSLRSEDSAMY | GGGSGTDYSLTISNLEPEDIATYFCHQYSKLP |
| | | | | FCTRVKLGTYYFDSWGQGTTLTVSSASTKGP | WTFGGGTKLEIKRTVAAPSVFIFPPSDEQLKS |
| | | | | SVFPLAPSSKSTSGGTAALGCLVKDYFPEPVT | GTASVVCLLNNFYPREAKVQWKVDNALQSG |
| | | | | VSWNSGALTSGVHTFPAVLQSSGLYSLSSVVT | NSQESVTEQDSKDSTYSLSSTLTLSKADYEK |
| | | | | VPSSSLGTQTYICNVNHKPSNTKVDKKVEPKS | HKVYACEVTHQGLSSPVTKSFNRGEC |
| | | | | CDKTHTCPPCPAPELLGGPSVFLFPPKPKDTL | |
| | | | | MISRTPEVTCVVVDVSHEDPEVKFNWYVDGV | |
| | | | | EVHNAKTKPREEQYNSTYRVVSVLTVLHQDW | |
| | | | | LNGKEYKCKVSNKALPAPIEKTISKAKGQPREP | |
| | | | | QVYTLPPSRDELTKNQVSLTCLVKGFYPSDIA | |
| | | | | VEWESNGQPENNYKTTPPVLDSDGSFFLYSK | |
| | | | | LTVDKSRWQQGNVFSCSVMHEALHNHYTQK | |
| | | | | SLSLSPGK | |
| Gelatinase | andecaliximab | | 709 | QVQLQESGPGLVKPSETLSLTCTVSGFSLLSY | DIQMTQSPSSLSASVGDRVTITCKASQDVRN |
| B | | | | GVHWVRQPPGKGLEWLGVIWTGGTTNYNSA | TVAWYQQKPGKAPKLLIYSSSYRNTGVPDR |
| | | | | LMSRFTISKDDSKNTVYLKMNSLKTEDTAIYYC | FSGSGSGTDFTLTISSLQAEDVAVYYCQQHY |
| | | | | ARYYYGMDYWGQGTLVTVSSASTKGPSVFPL | ITPYTFGGGTKVEIKRTVAAPSVFIFPPSDEQL |
| | | | | APCSRSTSESTAALGCLVKDYFPEPVTVSWN | KSGTASVVCLLNNFYPREAKVQWKVDNALQ |
| | | | | SGALTSGVHTFPAVLQSSGLYSLSSVVTVPSS | SGNSQESVTEQDSKDSTYSLSSTLTLSKADY |
| | | | | SLGTKTYTCNVDHKPSNTKVDKRVESKYGPP | EKHKVYACEVTHQGLSSPVTKSFNRGEC |
| | | | | CPPCPAPEFLGGPSVFLFPPKPKDTLMISRTP | |
| | | | | EVTCVVVDVSQEDPEVQFNWYVDGVEVHNA | |
| | | | | KTKPREEQFNSTYRVVSVLTVLHQDWLNGKE | |
| | | | | YKCKVSNKGLPSSIEKTISKAKGQPREPQVYTL | |
| | | | | PPSQEEMTKNQVSLTCLVKGFYPSDIAVEWES | |
| | | | | NGQPENNYKTTPPVLDSDGSFFLYSRLTVDKS | |
| | | | | RWQEGNVFSCSVMHEALHNHYTQKSLSLSLG | |
| | | | | K | |
| Glypican 3 | codrituzumab | 710 | 711 | QVQLVQSGAEVKKPGASVKVSCKASGYTFTD | DVVMTQSPLSLPVTPGEPASISCRSSQSLVH |
| | | | | YEMHWVRQAPGQGLEWMGALDPKTGDTAYS | SNRNTYLHWYLQKPGQSPQLLIYKVSNRFS |
| | | | | QKFKGRVTLTADKSTSTAYMELSSLTSEDTAV | GVPDRFSGSGSGTDFTLKISRVEAEDVGVYY |
| | | | | YYCTRFYSYTYWGQGTLVTVSSASTKGPSVF | CSQNTHVPPTFGQGTKLEIKRTVAAPSVFIFP |
| | | | | PLAPSSKSTSGGTAALGCLVKDYFPEPVTVSW | PSDEQLKSGTASVVCLLNNFYPREAKVQWKV |
| | | | | NSGALTSGVHTFPAVLQSSGLYSLSSVVTVPS | DNALQSGNSQESVTEQDSKDSTYSLSSTLTL |
| | | | | SSLGTQTYICNVNHKPSNTKVDKKVEPKSCDK | SKADYEKHKVYACEVTHQGLSSPVTKSFNRG |
| | | | | THTCPPCPAPELLGGPSVFLFPPKPKDTLMIS | EC |
| | | | | RTPEVTCVVVDVSHEDPEVKFNWYVDGVEVH | |
| | | | | NAKTKPREEQYNSTYRVVSVLTVLHQDWLNG | |
| | | | | KEYKCKVSNKALPAPIEKTISKAKGQPREPQV | |
| | | | | YTLPPSRDELTKNQVSLTCLVKGFYPSDIAVE | |
| | | | | WESNGQPENNYKTTPPVLDSDGSFFLYSKLT | |
| | | | | VDKSRWQQGNVFSCSVMHEALHNHYTQKSL | |
| | | | | SLSPGK | |
| GUCY2C | indusatumab | 712 | 713 | QVQLQQWGAGLLKPSETLSLTCAVFGGSFSG | EIVMTQSPATLSVSPGERATLSCRASQSVSR |
| | | | | YYWSWIRQPPGKGLEWIGEINHRGNTNDNPS | NLAWYQQKPGQAPRLLIYGASTRATGIPARF |
| | | | | LKSRVTISVDTSKNQFALKLSSVTAADTAVYYC | SGSGSGTEFTLTIGSLQSEDFAVYYCQQYKT |
| | | | | ARERGYTYGNFDHWGQGTLVTVSSASTKGPS | WPRTFGQGTNVEIKRTVAAPSVFIFPPSDEQL |
| | | | | VFPLAPSSKSTSGGTAALGCLVKDYFPEPVTV | KSGTASVVCLLNNFYPREAKVQWKVDNALQ |
| | | | | SWNSGALTSGVHTFPAVLQSSGLYSLSSVVTV | SGNSQESVTEQDSKDSTYSLSSTLTLSKADY |
| | | | | PSSSLGTQTYICNVNHKPSNTKVDKKVEPKSC | EKHKVYACEVTHQGLSSPVTKSFNRGEC |
| | | | | DKTHTCPPCPAPELLGGPSVFLFPPKPKDTLM | |
| | | | | ISRTPEVTCVVVDVSHEDPEVKFNWYVDGVE | |
| | | | | VHNAKTKPREEQYNSTYRVVSVLTVLHQDWL | |
| | | | | NGKEYKCKVSNKALPAPIEKTISKAKGQPREP | |
| | | | | QVYTLPPSRDELTKNQVSLTCLVKGFYPSDIA | |
| | | | | VEWESNGQPENNYKTTPPVLDSDGSFFLYSK | |
| | | | | LTVDKSRWQQGNVFSCSVMHEALHNHYTQK | |
| | | | | SLSLSPGK | |
| Hepatocyte | emibetuzumab | 714 | 715 | QVQLVQSGAEVKKPGASVKVSCKASGYTFTD | DIQMTQSPSSLSASVGDRVTITCSVSSSVSSI |
| growth | | | | YYMHWVRQAPGQGLEWMGRVNPNRRGTTY | YLHWYQQKPGKAPKLLIYSTSNLASGVPSRF |
| factor | | | | NQKFEGRVTMTTDTSTSTAYMELRSLRSDDT | SGSGSGTDFTLTISSLQPEDFATYYCQVYSG |
| receptor | | | | AVYYCARANWLDYWGQGTTVTVSSASTKGP | YPLTFGGGTKVEIKRTVAAPSVFIFPPSDEQL |
| (HGFR) | | | | SVFPLAPCSRSTSESTAALGCLVKDYFPEPVT | KSGTASVVCLLNNFYPREAKVQWKVDNALQ |
| | | | | VSWNSGALTSGVHTFPAVLQSSGLYSLSSVVT | SGNSQESVTEQDSKDSTYSLSSTLTLSKADY |
| | | | | VPSSSLGTKTYTCNVDHKPSNTKVDKRVESKY | EKHKVYACEVTHQGLSSPVTKSFNRGEC |
| | | | | GPPCPPCPAPEAAGGPSVFLFPPKPKDTLMIS | |
| | | | | RTPEVTCVVVDVSQEDPEVQFNWYVDGVEVH | |
| | | | | NAKTKPREEQFNSTYRVVSVLTVLHQDWLNG | |
| | | | | KEYKCKVSNKGLPSSIEKTISKAKGQPREPQV | |
| | | | | YTLPPSQEEMTKNQVSLTCLVKGFYPSDIAVE | |
| | | | | WESNGQPENNYKTTPPVLDSDGSFFLYSRLT | |
| | | | | VDKSRWQEGNVFSCSVMHEALHNHYTQKSL | |
| | | | | SLSLG | |
| HGFR | Amivantamab- | 716 | 717 | QVQLVQSGAEVKKPGASVKVSCETSGYTFTS | DIQMTQSPSSVSASVGDRVTITCRASQGISN |
| | bispecific | | | YGISWVRQAPGHGLEWMGWISAYNGYTNYA | WLAWFQHKPGKAPKLLIYAASSLLSGVPSR |
| | anti-EGFR | | | QKLQGRVTMTTDTSTSTAYMELRSLRSDDTA | FSGSGSGTDFTLTISSLQPEDFATYYCQQAN |
| | and anti- | | | VYYCARDLRGTNYFDYWGQGTLVTVSSASTK | SFPITFGQGTRLEIKRTVAAPSVFIFPPSDEQL |
| | HGFR | | | GPSVFPLAPSSKSTSGGTAALGCLVKDYFPEP | KSGTASVVCLLNNFYPREAKVQWKVDNALQ |
| | antibody | | | VTVSWNSGALTSGVHTFPAVLQSSGLYSLSSV | SGNSQESVTEQDSKDSTYSLSSTLTLSKADY |
| | | | | VTVPSSSLGTQTYICNVNHKPSNTKVDKRVEP | EKHKVYACEVTHQGLSSPVTKSFNRGEC |
| | | | | KSCDKTHTCPPCPAPELLGGPSVFLFPPKPKD | |
| | | | | TLMISRTPEVTCVVVDVSHEDPEVKFNWYVD | |
| | | | | GVEVHNAKTKPREEQYNSTYRVVSVLTVLHQ | |
| | | | | DWLNGKEYKCKVSNKALPAPIEKTISKAKGQP | |
| | | | | REPQVYTLPPSREEMTKNQVSLTCLVKGFYPS | |
| | | | | DIAVEWESNGQPENNYKTTPPVLDSDGSFFLY | |
| | | | | SRLTVDKSRWQQGNVFSCSVMHEALHNHYT | |
| | | | | QKSLSLSPGK | |
| HGFR | ficlatuzumab | 718 | 719 | QVQLVQPGAEVKKPGTSVKLSCKASGYTFTT | DIVMTQSPDSLAMSLGERVTLNCKASENVVS |
| | | | | YWMHWVRQAPGQGLEWIGEINPTNGHTNYN | YVSWYQQKPGQSPKLLIYGASNRESGVPDR |
| | | | | QKFQGRATLTVDKSTSTAYMELSSLRSEDTAV | FSGSGSATDFTLTISSVQAEDVADYHCGQSY |
| | | | | YYCARNYVGSIFDYWGQGTLLTVSSASTKGP | NYPYTFGQGTKLEIKRTVAAPSVFIFPPSDEQ |
| | | | | SVFPLAPSSKSTSGGTAALGCLVKDYFPEPVT | LKSGTASVVCLLNNFYPREAKVQWKVDNALQ |
| | | | | VSWNSGALTSGVHTFPAVLQSSGLYSLSSVVT | SGNSQESVTEQDSKDSTYSLSSTLTLSKADY |
| | | | | VPSSSLGTQTYICNVNHKPSNTKVDKRVEPKS | EKHKVYACEVTHQGLSSPVTKSFNRGEC |
| | | | | CDKTHTCPPCPAPELLGGPSVFLFPPKPKDTL | |
| | | | | MISRTPEVTCVVVDVSHEDPEVKFNWYVDGV | |
| | | | | EVHNAKTKPREEQYNSTYRVVSVLTVLHQDW | |
| | | | | LNGKEYKCKVSNKALPAPIEKTISKAKGQPREP | |
| | | | | QVYTLPPSREEMTKNQVSLTCLVKGFYPSDIA | |
| | | | | VEWESNGQPENNYKTTPPVLDSDGSFFLYSK | |
| | | | | LTVDKSRWQQGNVFSCSVMHEALHNHYTQK | |
| | | | | SLSLSPGK | |
| HGFR | Onartuzumab- | 720 | 721 | EVQLVESGGGLVQPGGSLRLSCAASGYTFTS | DIQMTQSPSSLSASVGDRVTITCKSSQSLLYT |
| | Heterodimeric | | | YWLHWVRQAPGKGLEWVGMIDPSNSDTRFN | SSQKNYLAWYQQKPGKAPKLLIYWASTRES |
| | Fab-Fc | | | PNFKDRFTISADTSKNTAYLQMNSLRAEDTAV | GVPSRFSGSGSGTDFTLTISSLQPEDFATYY |
| | | | | YYCATYRSYVTPLDYWGQGTLVTVSSASTKG | CQQYYAYPWTFGQGTKVEIKRTVAAPSVFIF |
| | | | | PSVFPLAPSSKSTSGGTAALGCLVKDYFPEPV | PPSDEQLKSGTASVVCLLNNFYPREAKVQWK |
| | | | | TVSWNSGALTSGVHTFPAVLQSSGLYSLSSVV | VDNALQSGNSQESVTEQDSKDSTYSLSSTLT |
| | | | | TVPSSSLGTQTYICNVNHKPSNTKVDKKVEPK | LSKADYEKHKVYACEVTHQGLSSPVTKSFNR |
| | | | | SCDKTHTCPPCPAPELLGGPSVFLFPPKPKDT | GEC |
| | | | | LMISRTPEVTCVVVDVSHEDPEVKFNWYVDG | |
| | | | | VEVHNAKTKPREEQYNSTYRVVSVLTVLHQD | |
| | | | | WLNGKEYKCKVSNKALPAPIEKTISKAKGQPR | |
| | | | | EPQVYTLPPSREEMTKNQVSLSCAVKGFYPS | |
| | | | | DIAVEWESNGQPENNYKTTPPVLDSDGSFFLV | |
| | | | | SKLTVDKSRWQQGNVFSCSVMHEALHNHYT | |
| | | | | QKSLSLSPGK | |
| HGFR | rilotumumab | 722 | 723 | QVQLQESGPGLVKPSETLSLTCTVSGGSISIYY | EIVMTQSPATLSVSPGERATLSCRASQSVDS |
| | | | | WSWIRQPPGKGLEWIGYVYYSGSTNYNPSLK | NLAWYRQKPGQAPRLLIYGASTRATGIPARF |
| | | | | SRVTISVDTSKNQFSLKLNSVTAADTAVYYCA | SGSGSGTEFTLTISSLQSEDFAVYYCQQYIN |
| | | | | RGGYDFWSGYFDYWGQGTLVTVSSASTKGP | WPPITFGQGTRLEIKRTVAAPSVFIFPPSDEQ |
| | | | | SVFPLAPCSRSTSESTAALGCLVKDYFPEPVT | LKSGTASVVCLLNNFYPREAKVQWKVDNALQ |
| | | | | VSWNSGALTSGVHTFPAVLQSSGLYSLSSVVT | SGNSQESVTEQDSKDSTYSLSSTLTLSKADY |
| | | | | VPSSNFGTQTYTCNVDHKPSNTKVDKTVERK | EKHKVYACEVTHQGLSSPVTKSFNRGEC |
| | | | | CCVECPPCPAPPVAGPSVFLFPPKPKDTLMIS | |
| | | | | RTPEVTCVVVDVSHEDPEVQFNWYVDGVEVH | |
| | | | | NAKTKPREEQFNSTFRVVSVLTVVHQDWLNG | |
| | | | | KEYKCKVSNKGLPAPIEKTISKTKGQPREPQV | |
| | | | | YTLPPSREEMTKNQVSLTCLVKGFYPSDIAVE | |
| | | | | WESNGQPENNYKTTPPMLDSDGSFFLYSKLT | |
| | | | | VDKSRWQQGNVFSCSVMHEALHNHYTQKSL | |
| | | | | SLSPGK | |
| HGFR | telisotuzumab | 724 | 725 | QVQLVQSGAEVKKPGASVKVSCKASGYIFTAY | DIVMTQSPDSLAVSLGERATINCKSSESVDS |
| | | | | TMHWVRQAPGQGLEWMGWIKPNNGLANYA | YANSFLHWYQQKPGQPPKLLIYRASTRESG |
| | | | | QKFQGRVTMTRDTSISTAYMELSRLRSDDTAV | VPDRFSGSGSGTDFTLTISSLQAEDVAVYYC |
| | | | | YYCARSEITTEFDYWGQGTLVTVSSASTKGPS | QQSKEDPLTFGGGTKVEIKRTVAAPSVFIFPP |
| | | | | VFPLAPSSKSTSGGTAALGCLVKDYFPEPVTV | SDEQLKSGTASVVCLLNNFYPREAKVQWKV |
| | | | | SWNSGALTSGVHTFPAVLQSSGLYSLSSVVTV | DNALQSGNSQESVTEQDSKDSTYSLSSTLTL |
| | | | | PSSSLGTQTYICNVNHKPSNTKVDKRVEPKSC | SKADYEKHKVYACEVTHQGLSSPVTKSFNRG |
| | | | | DCHCPPCPAPELLGGPSVFLFPPKPKDTLMIS | EC |
| | | | | RTPEVTCVVVDVSHEDPEVKFNWYVDGVEVH | |
| | | | | NAKTKPREEQYNSTYRVVSVLTVLHQDWLNG | |
| | | | | KEYKCKVSNKALPAPIEKTISKAKGQPREPQV | |
| | | | | YTLPPSREEMTKNQVSLTCLVKGFYPSDIAVE | |
| | | | | WESNGQPENNYKTTPPVLDSDGSFFLYSKLT | |
| | | | | VDKSRWQQGNVFSCSVMHEALHNHYTQKSL | |
| | | | | SLSPG | |
| HER2 | Gancotamab- | 726 | | QVQLVESGGGLVQPGGSLRLSCAASGFTFRS | |
| | scFv | | | YAMSWVRQAPGKGLEWVSAISGRGDNTYYA | |
| | | | | DSVKGRFTISRDNSKNTLYLQMNSLRAEDTAV | |
| | | | | YYCAKMTSNAFAFDYWGQGTLVTVSSGGGG | |
| | | | | SGGGGSGGGGSQSVLTQPPSVSGAPGQRVT | |
| | | | | ISCTGSSSNIGAGYGVHWYQQLPGTAPKLLIY | |
| | | | | GNTNRPSGVPDRFSGFKSGTSASLAITGLQAE | |
| | | | | DEADYYCQSYDSSLSGWVFGGGTKLTVLGGS | |
| | | | | GGC | |
| HER2 | margetuximab | 727 | 728 | QVQLQQSGPELVKPGASLKLSCTASGFNIKDT | DIVMTQSHKFMSTSVGDRVSITCKASQDVNT |
| | | | | YIHWVKQRPEQGLEWIGRIYPTNGYTRYDPKF | AVAWYQQKPGHSPKLLIYSASFRYTGVPDR |
| | | | | QDKATITADTSSNTAYLQVSRLTSEDTAVYYC | FTGSRSGTDFTFTISSVQAEDLAVYYCQQHY |
| | | | | SRWGGDGFYAMDYWGQGASVTVSSASTKGP | TTPPTFGGGTKVEIKRTVAAPSVFIFPPSDEQ |
| | | | | SVFPLAPSSKSTSGGTAALGCLVKDYFPEPVT | LKSGTASVVCLLNNFYPREAKVQWKVDNALQ |
| | | | | VSWNSGALTSGVHTFPAVLQSSGLYSLSSVVT | SGNSQESVTEQDSKDSTYSLSSTLTLSKADY |
| | | | | VPSSSLGTQTYICNVNHKPSNTKVDKRVEPKS | EKHKVYACEVTHQGLSSPVTKSFNRGEC |
| | | | | CDKTHTCPPCPAPELVGGPSVFLLPPKPKDTL | |
| | | | | MISRTPEVTCVVVDVSHEDPEVKFNWYVDGV | |
| | | | | EVHNAKTKPREEQYNSTLRVVSVLTVLHQDW | |
| | | | | LNGKEYKCKVSNKALPAPIEKTISKAKGQPREP | |
| | | | | QVYTLPPSRDELTKNQVSLTCLVKGFYPSDIA | |
| | | | | VEWESNGQPENNYKTTPLVLDSDGSFFLYSK | |
| | | | | LTVDKSRWQQGNVFSCSVMHEALHNHYTQK | |
| | | | | SLSLSPGK | |
| HER2 | pertuzumab | 729 | 730 | EVQLVESGGGLVQPGGSLRLSCAASGFTFTD | DIQMTQSPSSLSASVGDRVTITCKASQDVSIG |
| | | | | YTMDWVRQAPGKGLEWVADVNPNSGGSIYN | VAWYQQKPGKAPKLLIYSASYRYTGVPSRF |
| | | | | QRFKGRFTLSVDRSKNTLYLQMNSLRAEDTA | SGSGSGTDFTLTISSLQPEDFATYYCQQYYIY |
| | | | | VYYCARNLGPSFYFDYWGQGTLVTVSSASTK | PYTFGQGTKVEIKRTVAAPSVFIFPPSDEQLK |
| | | | | GPSVFPLAPSSKSTSGGTAALGCLVKDYFPEP | SGTASVVCLLNNFYPREAKVQWKVDNALQS |
| | | | | VTVSWNSGALTSGVHTFPAVLQSSGLYSLSSV | GNSQESVTEQDSKDSTYSLSSTLTLSKADYE |
| | | | | VTVPSSSLGTQTYICNVNHKPSNTKVDKKVEP | KHKVYACEVTHQGLSSPVTKSFNRGEC |
| | | | | KSCDKTHTCPPCPAPELLGGPSVFLFPPKPKD | |
| | | | | TLMISRTPEVTCVVVDVSHEDPEVKFNWYVD | |
| | | | | GVEVHNAKTKPREEQYNSTYRVVSVLTVLHQ | |
| | | | | DWLNGKEYKCKVSNKALPAPIEKTISKAKGQP | |
| | | | | REPQVYTLPPSREEMTKNQVSLTCLVKGFYPS | |
| | | | | DIAVEWESNGQPENNYKTTPPVLDSDGSFFLY | |
| | | | | SKLTVDKSRWQQGNVFSCSVMHEALHNHYT | |
| | | | | QKSLSLSPG | |
| HER2 | timigutuzumab | 731 | 732 | EVQLVESGGGLVQPGGSLRLSCAASGFNIKDT | DIQMTQSPSSLSASVGDRVTITCRASQDVNT |
| | | | | YIHWVRQAPGKGLEWVARIYPTNGYTRYADS | AVAWYQQKPGKAPKLLIYSASFLYSGVPSR |
| | | | | VKGRFTISADTSKNTAYLQMNSLRAEDTAVYY | FSGSRSGTDFTLTISSLQPEDFATYYCQQHY |
| | | | | CSRWGGDGFYAMDYWGQGTLVTVSSASTKG | TTPPTFGQGTKVEIKRTVAAPSVFIFPPSDEQ |
| | | | | PSVFPLAPSSKSTSGGTAALGCLVKDYFPEPV | LKSGTASVVCLLNNFYPREAKVQWKVDNALQ |
| | | | | TVSWNSGALTSGVHTFPAVLQSSGLYSLSSVV | SGNSQESVTEQDSKDSTYSLSSTLTLSKADY |
| | | | | TVPSSSLGTQTYICNVNHKPSNTKVDKKVEPK | EKHKVYACEVTHQGLSSPVTKSFNRGEC |
| | | | | SCDKTHTCPPCPAPELLGGPSVFLFPPKPKDT | |
| | | | | LMISRTPEVTCVVVDVSHEDPEVKFNWYVDG | |
| | | | | VEVHNAKTKPREEQYNSTYRVVSVLTVLHQD | |
| | | | | WLNGKEYKCKVSNKALPAPIEKTISKAKGQPR | |
| | | | | EPQVYTLPPSRDELTKNQVSLTCLVKGFYPSD | |
| | | | | IAVEWESNGQPENNYKTTPPVLDSDGSFFLYS | |
| | | | | KLTVDKSRWQQGNVFSCSVMHEGLHNHYTQ | |
| | | | | KSLSLSPGK | |
| HER2 | Trastuzumab | 733 | 734 | EVQLVESGGGLVQPGGSLRLSCAASGFNIKDT | DIQMTQSPSSLSASVGDRVTITCRASQDVNT |
| | | | | YIHWVRQAPGKGLEWVARIYPTNGYTRYADS | AVAWYQQKPGKAPKLLIYSASFLYSGVPSR |
| | | | | VKGRFTISADTSKNTAYLQMNSLRAEDTAVYY | FSGSRSGTDFTLTISSLQPEDFATYYCQQHY |
| | | | | CSRWGGDGFYAMDYWGQGTLVTVSSASTKG | TTPPTFGQGTKVEIKRTVAAPSVFIFPPSDEQ |
| | | | | PSVFPLAPSSKSTSGGTAALGCLVKDYFPEPV | LKSGTASVVCLLNNFYPREAKVQWKVDNALQ |
| | | | | TVSWNSGALTSGVHTFPAVLQSSGLYSLSSVV | SGNSQESVTEQDSKDSTYSLSSTLTLSKADY |
| | | | | TVPSSSLGTQTYICNVNHKPSNTKVDKKVEPK | EKHKVYACEVTHQGLSSPVTKSFNRGEC |
| | | | | SCDKTHTCPPCPAPELLGGPSVFLFPPKPKDT | |
| | | | | LMISRTPEVTCVVVDVSHEDPEVKFNWYVDG | |
| | | | | VEVHNAKTKPREEQYNSTYRVVSVLTVLHQD | |
| | | | | WLNGKEYKCKVSNKALPAPIEKTISKAKGQPR | |
| | | | | EPQVYTLPPSREEMTKNQVSLTCLVKGFYPSD | |
| | | | | IAVEWESNGQPENNYKTTPPVLDSDGSFFLYS | |
| | | | | KLTVDKSRWQQGNVFSCSVMHEALHNHYTQ | |
| | | | | KSLSLSPG | |
| HER3 | duligotuzumab | 735 | 736 | EVQLVESGGGLVQPGGSLRLSCAASGFTLSG | DIQMTQSPSSLSASVGDRVTITCRASQNIATD |
| | | | | DWIHWVRQAPGKGLEWVGEISAAGGYTDYAD | VAWYQQKPGKAPKLLIYSASFLYSGVPSRFS |
| | | | | SVKGRFTISADTSKNTAYLQMNSLRAEDTAVY | GSGSGTDFTLTISSLQPEDFATYYCQQSEPE |
| | | | | YCARESRVSFEAAMDYWGQGTLVTVSSASTK | PYTFGQGTKVEIKRTVAAPSVFIFPPSDEQLK |
| | | | | GPSVFPLAPSSKSTSGGTAALGCLVKDYFPEP | SGTASVVCLLNNFYPREAKVQWKVDNALQS |
| | | | | VTVSWNSGALTSGVHTFPAVLQSSGLYSLSSV | GNSQESVTEQDSKDSTYSLSSTLTLSKADYE |
| | | | | VTVPSSSLGTQTYICNVNHKPSNTKVDKKVEP | KHKVYACEVTHQGLSSPVTKSFNRGEC |
| | | | | KSCDKTHTCPPCPAPELLGGPSVFLFPPKPKD | |
| | | | | TLMISRTPEVTCVVVDVSHEDPEVKFNWYVD | |
| | | | | GVEVHNAKTKPREEQYNSTYRVVSVLTVLHQ | |
| | | | | DWLNGKEYKCKVSNKALPAPIEKTISKAKGQP | |
| | | | | REPQVYTLPPSREEMTKNQVSLTCLVKGFYPS | |
| | | | | DIAVEWESNGQPENNYKTTPPVLDSDGSFFLY | |
| | | | | SKLTVDKSRWQQGNVFSCSVMHEALHNHYT | |
| | | | | QKSLSLSPGK | |
| HER3 | elgemtumab | 737 | 738 | EVQLLESGGGLVQPGGSLRLSCAASGFTFSS | DIQMTQSPSSLSASVGDRVTITCRASQGISN |
| | | | | YAMSWVRQAPGKGLEWVSAINSQGKSTYYA | WLAWYQQKPGKAPKLLIYGASSLQSGVPSR |
| | | | | DSVKGRFTISRDNSKNTLYLQMNSLRAEDTAV | FSGSGSGTDFTLTISSLQPEDFATYYCQQYS |
| | | | | YYCARWGDEGFDIWGQGTLVTVSSASTKGPS | SFPTTFGQGTKVEIKRTVAAPSVFIFPPSDEQ |
| | | | | VFPLAPSSKSTSGGTAALGCLVKDYFPEPVTV | LKSGTASVVCLLNNFYPREAKVQWKVDNALQ |
| | | | | SWNSGALTSGVHTFPAVLQSSGLYSLSSVVTV | SGNSQESVTEQDSKDSTYSLSSTLTLSKADY |
| | | | | PSSSLGTQTYICNVNHKPSNTKVDKRVEPKSC | EKHKVYACEVTHQGLSSPVTKSFNRGEC |
| | | | | DKTHTCPPCPAPELLGGPSVFLFPPKPKDTLM | |
| | | | | ISRTPEVTCVVVDVSHEDPEVKFNWYVDGVE | |
| | | | | VHNAKTKPREEQYNSTYRVVSVLTVLHQDWL | |
| | | | | NGKEYKCKVSNKALPAPIEKTISKAKGQPREP | |
| | | | | QVYTLPPSREEMTKNQVSLTCLVKGFYPSDIA | |
| | | | | VEWESNGQPENNYKTTPPVLDSDGSFFLYSK | |
| | | | | LTVDKSRWQQGNVFSCSVMHEALHNHYTQK | |
| | | | | SLSLSPGK | |
| HER3 | lumretuzumab | 739 | 740 | QVQLVQSGAEVKKPGASVKVSCKASGYTFRS | DIVMTQSPDSLAVSLGERATINCKSSQSVLN |
| | | | | SYISWVRQAPGQGLEWMGWIYAGTGSPSYN | SGNQKNYLTWYQQKPGQPPKLLIYWASTRE |
| | | | | QKLQGRVTMTTDTSTSTAYMELRSLRSDDTA | SGVPDRFSGSGSGTDFTLTISSLQAEDVAVY |
| | | | | VYYCARHRDYYSNSLTYWGQGTLVTVSSAST | YCQSDYSYPYTFGQGTKLEIKRTVAAPSVFIF |
| | | | | KGPSVFPLAPSSKSTSGGTAALGCLVKDYFPE | PPSDEQLKSGTASVVCLLNNFYPREAKVQWK |
| | | | | PVTVSWNSGALTSGVHTFPAVLQSSGLYSLSS | VDNALQSGNSQESVTEQDSKDSTYSLSSTLT |
| | | | | VVTVPSSSLGTQTYICNVNHKPSNTKVDKKVE | LSKADYEKHKVYACEVTHQGLSSPVTKSFNR |
| | | | | PKSCDKTHTCPPCPAPELLGGPSVFLFPPKPK | GEC |
| | | | | DTLMISRTPEVTCVVVDVSHEDPEVKFNWYVD | |
| | | | | GVEVHNAKTKPREEQYNSTYRVVSVLTVLHQ | |
| | | | | DWLNGKEYKCKVSNKALPAPIEKTISKAKGQP | |
| | | | | REPQVYTLPPSRDELTKNQVSLTCLVKGFYPS | |
| | | | | DIAVEWESNGQPENNYKTTPPVLDSDGSFFLY | |
| | | | | SKLTVDKSRWQQGNVFSCSVMHEALHNHYT | |
| | | | | QKSLSLSPG | |
| HER3 | patritumab | 741 | 742 | QVQLQQWGAGLLKPSETLSLTCAVYGGSFSG | DIEMTQSPDSLAVSLGERATINCRSSQSVLY |
| | | | | YYWSWIRQPPGKGLEWIGEINHSGSTNYNPS | SSSNRNYLAWYQQNPGQPPKLLIYWASTRE |
| | | | | LKSRVTISVETSKNQFSLKLSSVTAADTAVYYC | SGVPDRFSGSGSGTDFTLTISSLQAEDVAVY |
| | | | | ARDKWTWYFDLWGRGTLVTVSSASTKGPSVF | YCQQYYSTPRTFGQGTKVEIKRTVAAPSVFIF |
| | | | | PLAPSSKSTSGGTAALGCLVKDYFPEPVTVSW | PPSDEQLKSGTASVVCLLNNFYPREAKVQWK |
| | | | | NSGALTSGVHTFPAVLQSSGLYSLSSVVTVPS | VDNALQSGNSQESVTEQDSKDSTYSLSSTLT |
| | | | | SSLGTQTYICNVNHKPSNTKVDKRVEPKSCDK | LSKADYEKHKVYACEVTHQGLSSPVTKSFNR |
| | | | | THTCPPCPAPELLGGPSVFLFPPKPKDTLMIS | GEC |
| | | | | RTPEVTCVVVDVSHEDPEVKFNWYVDGVEVH | |
| | | | | NAKTKPREEQYNSTYRVVSVLTVLHQDWLNG | |
| | | | | KEYKCKVSNKALPAPIEKTISKAKGQPREPQV | |
| | | | | YTLPPSREEMTKNQVSLTCLVKGFYPSDIAVE | |
| | | | | WESNGQPENNYKTTPPVLDSDGSFFLYSKLT | |
| | | | | VDKSRWQQGNVFSCSVMHEALHNHYTQKSL | |
| | | | | SLSPGK | |
| HER3 | seribantumab | 743 | 744 | EVQLLESGGGLVQPGGSLRLSCAASGFTFSH | QSALTQPASVSGSPGQSITISCTGTSSDVGS |
| | | | | YVMAWVRQAPGKGLEWVSSISSSGGWTLYA | YNVVSWYQQHPGKAPKLIIYEVSQRPSGVSN |
| | | | | DSVKGRFTISRDNSKNTLYLQMNSLRAEDTAV | RFSGSKSGNTASLTISGLQTEDEADYYCCSY |
| | | | | YYCTRGLKMATIFDYWGQGTLVTVSSASTKG | AGSSIFVIFGGGTKVTVLGQPKAAPSVTLFPP |
| | | | | PSVFPLAPCSRSTSESTAALGCLVKDYFPEPV | SSEELQANKATLVCLVSDFYPGAVTVAWKAD |
| | | | | TVSWNSGALTSGVHTFPAVLQSSGLYSLSSVV | GSPVKVGVETTKPSKQSNNKYAASSYLSLTP |
| | | | | TVPSSNFGTQTYTCNVDHKPSNTKVDKTVER | EQWKSHRSYSCRVTHEGSTVEKTVAPAECS |
| | | | | KCCVECPPCPAPPVAGPSVFLFPPKPKDTLMI | |
| | | | | SRTPEVTCVVVDVSHEDPEVQFNWYVDGVEV | |
| | | | | HNAKTKPREEQFNSTFRVVSVLTVVHQDWLN | |
| | | | | GKEYKCKVSNKGLPAPIEKTISKTKGQPREPQ | |
| | | | | VYTLPPSREEMTKNQVSLTCLVKGFYPSDIAV | |
| | | | | EWESNGQPENNYKTTPPMLDSDGSFFLYSKL | |
| | | | | TVDKSRWQQGNVFSCSVMHEALHNHYTQKS | |
| | | | | LSLSPGK | |
| HER2/HER3 | Zenocutuzumab- | 745 | 746 | QVQLVQSGAEVKKPGASVKVSCKASGYTFTG | DIQMTQSPSSLSASVGDRVTITCRASQSISSY |
| | bispecific | | | YYMHWVRQAPGQGLEWMGWINPNSGGTNY | LNWYQQKPGKAPKLLIYAASSLQSGVPSRF |
| | anti-HER2 | | | AQKFQGRVTMTRDTSISTAYMELSRLRSDDTA | SGSGSGTDFTLTISSLQPEDFATYYCQQSYS |
| | and anti- | | | VYYCARDHGSRHFWSYWGFDYWGQGTLVTV | TPPTFGQGTKVEIKRTVAAPSVFIFPPSDEQL |
| | HER3 | | | SSASTKGPSVFPLAPSSKSTSGGTAALGCLVK | KSGTASVVCLLNNFYPREAKVQWKVDNALQ |
| | antibody | | | DYFPEPVTVSWNSGALTSGVHTFPAVLQSSG | SGNSQESVTEQDSKDSTYSLSSTLTLSKADY |
| | | | | LYSLSSVVTVPSSSLGTQTYICNVNHKPSNTK | EKHKVYACEVTHQGLSSPVTKSFNRGEC |
| | | | | VDKRVEPKSCDKTHTCPPCPAPELLGGPSVFL | |
| | | | | FPPKPKDTLMISRTPEVTCVVVDVSHEDPEVK | |
| | | | | FNWYVDGVEVHNAKTKPREEQYNSTYRVVSV | |
| | | | | LTVLHQDWLNGKEYKCKVSNKALPAPIEKTISK | |
| | | | | AKGQPREPQVYTKPPSREEMTKNQVSLKCLV | |
| | | | | KGFYPSDIAVEWESNGQPENNYKTTPPVLDS | |
| | | | | DGSFFLYSKLTVDKSRWQQGNVFSCSVMHEA | |
| | | | | LHNHYTQKSLSLSPG | |
| IGF-1R | cixutumumab | 747 | 748 | EVQLVQSGAEVKKPGSSVKVSCKASGGTFSS | SSELTQDPAVSVALGQTVRITCQGDSLRSYY |
| | | | | YAISWVRQAPGQGLEWMGGIIPIFGTANYAQK | ATWYQQKPGQAPILVIYGENKRPSGIPDRFS |
| | | | | FQGRVTITADKSTSTAYMELSSLRSEDTAVYY | GSSSGNTASLTITGAQAEDEADYYCKSRDG |
| | | | | CARAPLRFLEWSTQDHYYYYYMDVWGKGTT | SGQHLVFGGGTKLTVLGQPKAAPSVTLFPPS |
| | | | | VTVSSASTKGPSVFPLAPSSKSTSGGTAALGC | SEELQANKATLVCLISDFYPGAVTVAWKADS |
| | | | | LVKDYFPEPVTVSWNSGALTSGVHTFPAVLQS | SPVKAGVETTTPSKQSNNKYAASSYLSLTPE |
| | | | | SGLYSLSSVVTVPSSSLGTQTYICNVNHKPSN | QWKSHRSYSCQVTHEGSTVEKTVAPAECS |
| | | | | TKVDKKVEPKSCDKTHTCPPCPAPELLGGPSV | |
| | | | | FLFPPKPKDTLMISRTPEVTCVVVDVSHEDPE | |
| | | | | VKFNWYVDGVEVHNAKTKPREEQYNSTYRVV | |
| | | | | SVLTVLHQDWLNGKEYKCKVSNKALPAPIEKTI | |
| | | | | SKAKGQPREPQVYTLPPSREEMTKNQVSLTC | |
| | | | | LVKGFYPSDIAVEWESNGQPENNYKTTPPVLD | |
| | | | | SDGSFFLYSKLTVDKSRWQQGNVFSCSVMHE | |
| | | | | ALHNHYTQKSLSLSPGK | |
| IGF-1R | dalotuzumab | 749 | 750 | QVQLQESGPGLVKPSETLSLTCTVSGYSITGG | DIVMTQSPLSLPVTPGEPASISCRSSQSIVHS |
| | | | | YLWNWIRQPPGKGLEWIGYISYDGTNNYKPSL | NGNTYLQWYLQKPGQSPQLLIYKVSNRLYG |
| | | | | KDRVTISRDTSKNQFSLKLSSVTAADTAVYYC | VPDRFSGSGSGTDFTLKISRVEAEDVGVYYC |
| | | | | ARYGRVFFDYWGQGTLVTVSSASTKGPSVFP | FQGSHVPWTFGQGTKVEIKRTVAAPSVFIFP |
| | | | | LAPSSKSTSGGTAALGCLVKDYFPEPVTVSW | PSDEQLKSGTASVVCLLNNFYPREAKVQWKV |
| | | | | NSGALTSGVHTFPAVLQSSGLYSLSSVVTVPS | DNALQSGNSQESVTEQDSKDSTYSLSSTLTL |
| | | | | SSLGTQTYICNVNHKPSNTKVDKRVEPKSCDK | SKADYEKHKVYACEVTHQGLSSPVTKSFNRG |
| | | | | THTCPPCPAPELLGGPSVFLFPPKPKDTLMIS | EC |
| | | | | RTPEVTCVVVDVSHEDPEVKFNWYVDGVEVH | |
| | | | | NAKTKPREEQYNSTYRVVSVLTVLHQDWLNG | |
| | | | | KEYKCKVSNKALPAPIEKTISKAKGQPREPQV | |
| | | | | YTLPPSREEMTKNQVSLTCLVKGFYPSDIAVE | |
| | | | | WESNGQPENNYKTTPPVLDSDGSFFLYSKLT | |
| | | | | VDKSRWQQGNVFSCSVMHEALHNHYTQKSL | |
| | | | | SLSPGK | |
| IGF-1R | Figitumumab | 751 | 752 | EVQLLESGGGLVQPGGSLRLSCTASGFTFSS | DIQMTQFPSSLSASVGDRVTITCRASQGIRN |
| | | | | YAMNWVRQAPGKGLEWVSAISGSGGTTFYA | DLGWYQQKPGKAPKRLIYAASRLHRGVPSR |
| | | | | DSVKGRFTISRDNSRTTLYLQMNSLRAEDTAV | FSGSGSGTEFTLTISSLQPEDFATYYCLQHN |
| | | | | YYCAKDLGWSDSYYYYYGMDVWGQGTTVTV | SYPCSFGQGTKLEIKRTVAAPSVFIFPPSDEQ |
| | | | | SSASTKGPSVFPLAPCSRSTSESTAALGCLVK | LKSGTASVVCLLNNFYPREAKVQWKVDNALQ |
| | | | | DYFPEPVTVSWNSGALTSGVHTFPAVLQSSG | SGNSQESVTEQDSKDSTYSLSSTLTLSKADY |
| | | | | LYSLSSVVTVPSSNFGTQTYTCNVDHKPSNTK | EKHKVYACEVTHQGLSSPVTKSFNRGEC |
| | | | | VDKTVERKCCVECPPCPAPPVAGPSVFLFPPK | |
| | | | | PKDTLMISRTPEVTCVVVDVSHEDPEVQFNW | |
| | | | | YVDGVEVHNAKTKPREEQFNSTFRVVSVLTVV | |
| | | | | HQDWLNGKEYKCKVSNKGLPAPIEKTISKTKG | |
| | | | | QPREPQVYTLPPSREEMTKNQVSLTCLVKGF | |
| | | | | YPSDIAVEWESNGQPENNYKTTPPMLDSDGS | |
| | | | | FFLYSKLTVDKSRWQQGNVFSCSVMHEALHN | |
| | | | | HYTQKSLSLSPG | |
| IGF-1R | ganitumab | 753 | 754 | QVQLQESGPGLVKPSGTLSLTCAVSGGSISSS | DVVMTQSPLSLPVTPGEPASISCRSSQSLLH |
| | | | | NWWSWVRQPPGKGLEWIGEIYHSGSTNYNP | SNGYNYLDWYLQKPGQSPQLLIYLGSNRAS |
| | | | | SLKSRVTISVDKSKNQFSLKLSSVTAADTAVYY | GVPDRFSGSGSGTDFTLKISRVEAEDVGVYY |
| | | | | CARWTGRTDAFDIWGQGTMVTVSSASTKGPS | CMQGTHWPLTFGQGTKVEIKRTVAAPSVFIF |
| | | | | VFPLAPSSKSTSGGTAALGCLVKDYFPEPVTV | PPSDEQLKSGTASVVCLLNNFYPREAKVQWK |
| | | | | SWNSGALTSGVHTFPAVLQSSGLYSLSSVVTV | VDNALQSGNSQESVTEQDSKDSTYSLSSTLT |
| | | | | PSSSLGTQTYICNVNHKPSNTKVDKKVEPKSC | LSKADYEKHKVYACEVTHQGLSSPVTKSFNR |
| | | | | DKTHTCPPCPAPELLGGPSVFLFPPKPKDTLM | GEC |
| | | | | ISRTPEVTCVVVDVSHEDPEVKFNWYVDGVE | |
| | | | | VHNAKTKPREEQYNSTYRVVSVLTVLHQDWL | |
| | | | | NGKEYKCKVSNKALPAPIEKTISKAKGQPREP | |
| | | | | QVYTLPPSRDELTKNQVSLTCLVKGFYPSDIA | |
| | | | | VEWESNGQPENNYKTTPPVLDSDGSFFLYSK | |
| | | | | LTVDKSRWQQGNVFSCSVMHEALHNHYTQK | |
| | | | | SLSLSPGK | |
| IGF-1R | robatumumab | 755 | 756 | EVQLVQSGGGLVKPGGSLRLSCAASGFTFSS | EIVLTQSPGTLSVSPGERATLSCRASQSIGSS |
| | | | | FAMHWVRQAPGKGLEWISVIDTRGATYYADS | LHWYQQKPGQAPRLLIKYASQSLSGIPDRFS |
| | | | | VKGRFTISRDNAKNSLYLQMNSLRAEDTAVYY | GSGSGTDFTLTISRLEPEDFAVYYCHQSSRL |
| | | | | CARLGNFYYGMDVWGQGTTVTVSSASTKGP | PHTFGQGTKVEIKRTVAAPSVFIFPPSDEQLK |
| | | | | SVFPLAPSSKSTSGGTAALGCLVKDYFPEPVT | SGTASVVCLLNNFYPREAKVQWKVDNALQS |
| | | | | VSWNSGALTSGVHTFPAVLQSSGLYSLSSVVT | GNSQESVTEQDSKDSTYSLSSTLTLSKADYE |
| | | | | VPSSSLGTQTYICNVNHKPSNTKVDKKVEPKS | KHKVYACEVTHQGLSSPVTKSFNRGEC |
| | | | | CDKTHTCPPCPAPELLGGPSVFLFPPKPKDTL | |
| | | | | MISRTPEVTCVVVDVSHEDPEVKFNWYVDGV | |
| | | | | EVHNAKTKPREEQYNSTYRVVSVLTVLHQDW | |
| | | | | LNGKEYKCKVSNKALPAPIEKTISKAKGQPREP | |
| | | | | QVYTLPPSRDELTKNQVSLTCLVKGFYPSDIA | |
| | | | | VEWESNGQPENNYKTTPPVLDSDGSFFLYSK | |
| | | | | LTVDKSRWQQGNVFSCSVMHEALHNHYTQK | |
| | | | | SLSLSPGK | |
| IGF-1R | teprotumumab | 757 | 758 | QVELVESGGGVVQPGRSQRLSCAASGFTFSS | EIVLTQSPATLSLSPGERATLSCRASQSVSS |
| | | | | YGMHWVRQAPGKGLEWVAIIWFDGSSTYYAD | YLAWYQQKPGQAPRLLIYDASKRATGIPARF |
| | | | | SVRGRFTISRDNSKNTLYLQMNSLRAEDTAVY | SGSGSGTDFTLTISSLEPEDFAVYYCQQRSK |
| | | | | FCARELGRRYFDLWGRGTLVSVSSASTKGPS | WPPWTFGQGTKVESKRTVAAPSVFIFPPSDE |
| | | | | VFPLAPSSKSTSGGTAALGCLVKDYFPEPVTV | QLKSGTASVVCLLNNFYPREAKVQWKVDNAL |
| | | | | SWNSGALTSGVHTFPAVLQSSGLYSLSSVVTV | QSGNSQESVTEQDSKDSTYSLSSTLTLSKAD |
| | | | | PSSSLGTQTYICNVNHKPSNTKVDKKVEPKSC | YEKHKVYACEVTHQGLSSPVTKSFNRGEC |
| | | | | DKTHTCPPCPAPELLGGPSVFLFPPKPKDTLM | |
| | | | | ISRTPEVTCVVVDVSHEDPEVKFNWYVDGVE | |
| | | | | VHNAKTKPREEQYNSTYRVVSVLTVLHQDWL | |
| | | | | NGKEYKCKVSNKALPAPIEKTISKAKGQPREP | |
| | | | | QVYTLPPSRDELTKNQVSLTCLVKGFYPSDIA | |
| | | | | VEWESNGQPENNYKTTPPVLDSDGSFFLYSK | |
| | | | | LTVDKSRWQQGNVFSCSVMHEALHNHYTQK | |
| | | | | SLSLSPGK | |
| IGF-1R/ | xentuzumab | 759 | 760 | QVELVESGGGLVQPGGSLRLSCAASGFTFTS | DIVLTQPPSVSGAPGQRVTISCSGSSSNIGSN |
| IGF-2R | | | | YWMSWVRQAPGKGLELVSSITSYGSFTYYAD | SVSWYQQLPGTAPKLLIYDNSKRPSGVPDR |
| | | | | SVKGRFTISRDNSKNTLYLQMNSLRAEDTAVY | FSGSKSGTSASLAITGLQSEDEADYYCQSRD |
| | | | | YCARNMYTHFDSWGQGTLVTVSSASTKGPSV | TYGYYWVFGGGTKLTVLGQPKAAPSVTLFPP |
| | | | | FPLAPSSKSTSGGTAALGCLVKDYFPEPVTVS | SSEELQANKATLVCLISDFYPGAVTVAWKGD |
| | | | | WNSGALTSGVHTFPAVLQSSGLYSLSSVVTVP | SSPVKAGVETTTPSKQSNNKYAASSYLSLTP |
| | | | | SSSLGTQTYICNVNHKPSNTKVDKKVEPKSCD | EQWKSHRSYSCQVTHEGSTVEKTVAPTECS |
| | | | | KTHTCPPCPAPELLGGPSVFLFPPKPKDTLMIS | |
| | | | | RTPEVTCVVVDVSHEDPEVKFNWYVDGVEVH | |
| | | | | NAKTKPREEQYNSTYRVVSVLTVLHQDWLNG | |
| | | | | KEYKCKVSNKALPAPIEKTISKAKGQPREPQV | |
| | | | | YTLPPSRDELTKNQVSLTCLVKGFYPSDIAVE | |
| | | | | WESNGQPENNYKTTPPVLDSDGSFFLYSKLT | |
| | | | | VDKSRWQQGNVFSCSVMHEALHNHYTQKSL | |
| | | | | SLSPGK | |
| IGF-2R | dusigitumab | 761 | 762 | QVQLVQSGAEVKKPGASVKVSCKASGYTFTS | QSVLTQPPSVSAAPGQKVTISCSGSSSNIEN |
| | | | | YDINWVRQATGQGLEWMGWMNPNSGNTGY | NHVSWYQQLPGTAPKLLIYDNNKRPSGIPDR |
| | | | | AQKFQGRVTMTRNTSISTAYMELSSLRSEDTA | FSGSKSGTSATLGITGLQTGDEADYYCETW |
| | | | | VYYCARDPYYYYYGMDVWGQGTTVTVSSAS | DTSLSAGRVFGGGTKLTVLGQPKAAPSVTLF |
| | | | | TKGPSVFPLAPCSRSTSESTAALGCLVKDYFP | PPSSEELQANKATLVCLISDFYPGAVTVAWKA |
| | | | | EPVTVSWNSGALTSGVHTFPAVLQSSGLYSLS | DSSPVKAGVETTTPSKQSNNKYAASSYLSLT |
| | | | | SVVTVPSSNFGTQTYTCNVDHKPSNTKVDKT | PEQWKSHRSYSCQVTHEGSTVEKTVAPTEC |
| | | | | VERKCCVECPPCPAPPVAGPSVFLFPPKPKDT | S |
| | | | | LMISRTPEVTCVVVDVSHEDPEVQFNWYVDG | |
| | | | | VEVHNAKTKPREEQFNSTFRVVSVLTVVHQD | |
| | | | | WLNGKEYKCKVSNKGLPAPIEKTISKTKGQPR | |
| | | | | EPQVYTLPPSREEMTKNQVSLTCLVKGFYPSD | |
| | | | | IAVEWESNGQPENNYKTTPPMLDSDGSFFLY | |
| | | | | SKLTVDKSRWQQGNVFSCSVMHEALHNHYT | |
| | | | | QKSLSLSPGK | |
| Integrin | etaracizumab | 763 | 764 | QVQLVESGGGVVQPGRSLRLSCAASGFTFSS | EIVLTQSPATLSLSPGERATLSCQASQSISNF |
| αvβ3 | | | | YDMSWVRQAPGKGLEWVAKVSSGGGSTYYL | LHWYQQRPGQAPRLLIRYRSQSISGIPARFS |
| | | | | DTVQGRFTISRDNSKNTLYLQMNSLRAEDTAV | GSGSGTDFTLTISSLEPEDFAVYYCQQSGSW |
| | | | | YYCARHLHGSFASWGQGTTVTVSSASTKGPS | PLTFGGGTKVEIKRTVAAPSVFIFPPSDEQLK |
| | | | | VFPLAPSSKSTSGGTAALGCLVKDYFPEPVTV | SGTASVVCLLNNFYPREAKVQWKVDNALQS |
| | | | | SWNSGALTSGVHTFPAVLQSSGLYSLSSVVTV | GNSQESVTEQDSKDSTYSLSSTLTLSKADYE |
| | | | | PSSSLGTQTYICNVNHKPSNTKVDKRVEPKSC | KHKVYACEVTHQGLSSPVTKSFNRGEC |
| | | | | DKTHTCPPCPAPELLGGPSVFLFPPKPKDTLM | |
| | | | | ISRTPEVTCVVVDVSHEDPEVKFNWYVDGVE | |
| | | | | VHNAKTKPREEQYNSTYRVVSVLTVLHQDWL | |
| | | | | NGKEYKCKVSNKALPAPIEKTISKAKGQPREP | |
| | | | | QVYTLPPSREEMTKNQVSLTCLVKGFYPSDIA | |
| | | | | VEWESNGQPENNYKTTPPVLDSDGSFFLYSK | |
| | | | | LTVDKSRWQQGNVFSCSVMHEALHNHYTQK | |
| | | | | SLSLSPGK | |
| Integrin α₅ | volociximab | 765 | 766 | QVQLKESGPGLVAPSQSLSITCTISGFSLTDYG | QIVLTQSPAIMSASLGERVTMTCTASSSVSS |
| β₁ | | | | VHWVRQPPGKGLEWLVVIWSDGSSTYNSALK | NYLHWYQQKPGSAPNLWIYSTSNLASGVPA |
| | | | | SRMTIRKDNSKSQVFLIMNSLQTDDSAMYYCA | RFSGSGSGTSYSLTISSMEAEDAATYYCHQY |
| | | | | RHGTYYGMTTTGDALDYWGQGTSVTVSSAST | LRSPPTFGGGTKLEIKRTVAAPSVFIFPPSDE |
| | | | | KGPSVFPLAPCSRSTSESTAALGCLVKDYFPE | QLKSGTASVVCLLNNFYPREAKVQWKVDNAL |
| | | | | PVTVSWNSGALTSGVHTFPAVLQSSGLYSLSS | QSGNSQESVTEQDSKDSTYSLSSTLTLSKAD |
| | | | | VVTVPSSSLGTKTYTCNVDHKPSNTKVDKRVE | YEKHKVYACEVTHQGLSSPVTKSFNRGEC |
| | | | | SKYGPPCPSCPAPEFLGGPSVFLFPPKPKDTL | |
| | | | | MISRTPEVTCVVVDVSQEDPEVQFNWYVDGV | |
| | | | | EVHNAKTKPREEQFNSTYRVVSVLTVLHQDW | |
| | | | | LNGKEYKCKVSNKGLPSSIEKTISKAKGQPRE | |
| | | | | PQVYTLPPSQEEMTKNQVSLTCLVKGFYPSDI | |
| | | | | AVEWESNGQPENNYKTTPPVLDSDGSFFLYS | |
| | | | | RLTVDKSRWQEGNVFSCSVMHEALHNHYTQ | |
| | | | | KSLSLSLGK | |
| IL-3R | Flotetuzumab- | 767 | 768 | QAVVTQEPSLTVSPGGTVTLTCRSSTGAVTTS | DFVMTQSPDSLAVSLGERVTMSCKSSQSLL |
| | anti- | | | NYANWVQQKPGQAPRGLIGGTNKRAPWTPA | NSGNQKNYLTWYQQKPGQPPKLLIYWASTR |
| | CD3 and | | | RFSGSLLGGKAALTITGAQAEDEADYYCALWY | ESGVPDRFSGSGSGTDFTLTISSLQAEDVAV |
| | anti-IL-3R | | | SNLWVFGGGTKLTVLGGGGSGGGGEVQLVQ | YYCQNDYSYPYTFGQGTKLEIKGGGSGGGG |
| | diabody | | | SGAELKKPGASVKVSCKASGYTFTDYYMKWV | EVQLVESGGGLVQPGGSLRLSCAASGFTFST |
| | | | | RQAPGQGLEWIGDIIPSNGATFYNQKFKGRVT | YAMNWVRQAPGKGLEWVGRIRSKYNNYATY |
| | | | | ITVDKSTSTAYMELSSLRSEDTAVYYCARSHLL | YADSVKDRFTISRDDSKNSLYLQMNSLKTED |
| | | | | RASWFAYWGQGTLVTVSSGGGGGGEVAALE | TAVYYCVRHGNFGNSYVSWFAYWGQGTLVT |
| | | | | KEVAALEKEVAALEKEVAALEK | VSSGGCGGGKVAALKEKVAALKEKVAALKEK |
| | | | | | VAALKE |
| KIR2D | lirilumab | 769 | 770 | QVQLVQSGAEVKKPGSSVKVSCKASGGTFSF | EIVLTQSPVTLSLSPGERATLSCRASQSVSS |
| | | | | YAISWVRQAPGQGLEWMGGFIPIFGAANYAQ | YLAWYQQKPGQAPRLLIYDASNRATGIPARF |
| | | | | KFQGRVTITADESTSTAYMELSSLRSDDTAVY | SGSGSGTDFTLTISSLEPEDFAVYYCQQRSN |
| | | | | YCARIPSGSYYYDYDMDVWGQGTTVTVSSAS | WMYTFGQGTKLEIKRTVAAPSVFIFPPSDEQ |
| | | | | TKGPSVFPLAPCSRSTSESTAALGCLVKDYFP | LKSGTASVVCLLNNFYPREAKVQWKVDNALQ |
| | | | | EPVTVSWNSGALTSGVHTFPAVLQSSGLYSLS | SGNSQESVTEQDSKDSTYSLSSTLTLSKADY |
| | | | | SVVTVPSSSLGTKTYTCNVDHKPSNTKVDKRV | EKHKVYACEVTHQGLSSPVTKSFNRGEC |
| | | | | ESKYGPPCPPCPAPEFLGGPSVFLFPPKPKDT | |
| | | | | LMISRTPEVTCVVVDVSQEDPEVQFNWYVDG | |
| | | | | VEVHNAKTKPREEQFNSTYRVVSVLTVLHQD | |
| | | | | WLNGKEYKCKVSNKGLPSSIEKTISKAKGQPR | |
| | | | | EPQVYTLPPSQEEMTKNQVSLTCLVKGFYPS | |
| | | | | DIAVEWESNGQPENNYKTTPPVLDSDGSFFLY | |
| | | | | SRLTVDKSRWQEGNVFSCSVMHEALHNHYT | |
| | | | | QKSLSLSLGK | |
| LIV-1 | ladiratuzumab | 771 | 772 | QVQLVQSGAEVKKPGASVKVSCKASGLTIEDY | DVVMTQSPLSLPVTLGQPASISCRSSQSLLH |
| | | | | YMHWVRQAPGQGLEWMGWIDPENGDTEYG | SSGNTYLEWYQQRPGQSPRPLIYKISTRFSG |
| | | | | PKFQGRVTMTRDTSINTAYMELSRLRSDDTAV | VPDRFSGSGSGTDFTLKISRVEAEDVGVYYC |
| | | | | YYCAVHNAHYGTWFAYWGQGTLVTVSSASTK | FQGSHVPYTFGGGTKVEIKRTVAAPSVFIFPP |
| | | | | GPSVFPLAPSSKSTSGGTAALGCLVKDYFPEP | SDEQLKSGTASVVCLLNNFYPREAKVQWKV |
| | | | | VTVSWNSGALTSGVHTFPAVLQSSGLYSLSSV | DNALQSGNSQESVTEQDSKDSTYSLSSTLTL |
| | | | | VTVPSSSLGTQTYICNVNHKPSNTKVDKKVEP | SKADYEKHKVYACEVTHQGLSSPVTKSFNRG |
| | | | | KSCDKTHTCPPCPAPELLGGPSVFLFPPKPKD | EC |
| | | | | TLMISRTPEVTCVVVDVSHEDPEVKFNWYVD | |
| | | | | GVEVHNAKTKPREEQYNSTYRVVSVLTVLHQ | |
| | | | | DWLNGKEYKCKVSNKALPAPIEKTISKAKGQP | |
| | | | | REPQVYTLPPSRDELTKNQVSLTCLVKGFYPS | |
| | | | | DIAVEWESNGQPENNYKTTPPVLDSDGSFFLY | |
| | | | | SKLTVDKSRWQQGNVFSCSVMHEALHNHYT | |
| | | | | QKSLSLSPGK | |
| LRRC15 | samrotamab | 773 | 774 | EVQLVQSGAEVKKPGASVKVSCKASGYKFSS | DIQMTQSPSSLSASVGDRVTITCRASQDISNY |
| | | | | YWIEWVKQAPGQGLEWIGEILPGSDTTNYNEK | LNWYQQKPGGAVKFLIYYTSRLHSGVPSRF |
| | | | | FKDRATFTSDTSINTAYMELSRLRSDDTAVYY | SGSGSGTDYTLTISSLQPEDFATYFCQQGEA |
| | | | | CARDRGNYRAWFGYWGQGTLVTVSSASTKG | LPWTFGGGTKVEIKRTVAAPSVFIFPPSDEQL |
| | | | | PSVFPLAPSSKSTSGGTAALGCLVKDYFPEPV | KSGTASVVCLLNNFYPREAKVQWKVDNALQ |
| | | | | TVSWNSGALTSGVHTFPAVLQSSGLYSLSSVV | SGNSQESVTEQDSKDSTYSLSSTLTLSKADY |
| | | | | TVPSSSLGTQTYICNVNHKPSNTKVDKKVEPK | EKHKVYACEVTHQGLSSPVTKSFNRGEC |
| | | | | SCDKTHTCPPCPAPELLGGPSVFLFPPKPKDT | |
| | | | | LMISRTPEVTCVVVDVSHEDPEVKFNWYVDG | |
| | | | | VEVHNAKTKPREEQYNSTYRVVSVLTVLHQD | |
| | | | | WLNGKEYKCKVSNKALPAPIEKTISKAKGQPR | |
| | | | | EPQVYTLPPSREEMTKNQVSLTCLVKGFYPSD | |
| | | | | IAVEWESNGQPENNYKTTPPVLDSDGSFFLYS | |
| | | | | KLTVDKSRWQQGNVFSCSVMHEALHNHYTQ | |
| | | | | KSLSLSPGK | |
| Mesothelin | amatuximab | 775 | 776 | QVQLQQSGPELEKPGASVKISCKASGYSFTG | DIELTQSPAIMSASPGEKVTMTCSASSSVSY |
| | | | | YTMNWVKQSHGKSLEWIGLITPYNGASSYNQ | MHWYQQKSGTSPKRWIYDTSKLASGVPGRF |
| | | | | KFRGKATLTVDKSSSTAYMDLLSLTSEDSAVY | SGSGSGNSYSLTISSVEAEDDATYYCQQWS |
| | | | | FCARGGYDGRGFDYWGSGTPVTVSSASTKG | KHPLTFGSGTKVEIKRTVAAPSVFIFPPSDEQ |
| | | | | PSVFPLAPSSKSTSGGTAALGCLVKDYFPEPV | LKSGTASVVCLLNNFYPREAKVQWKVDNALQ |
| | | | | TVSWNSGALTSGVHTFPAVLQSSGLYSLSSVV | SGNSQESVTEQDSKDSTYSLSSTLTLSKADY |
| | | | | TVPSSSLGTQTYICNVNHKPSNTKVDKKVEPK | EKHKVYACEVTHQGLSSPVTKSFNRGEC |
| | | | | SCDKTHTCPPCPAPELLGGPSVFLFPPKPKDT | |
| | | | | LMISRTPEVTCVVVDVSHEDPEVKFNWYVDG | |
| | | | | VEVHNAKTKPREEQYNSTYRVVSVLTVLHQD | |
| | | | | WLNGKEYKCKVSNKALPAPIEKTISKAKGQPR | |
| | | | | EPQVYTLPPSRDELTKNQVSLTCLVKGFYPSD | |
| | | | | IAVEWESNGQPENNYKTTPPVLDSDGSFFLYS | |
| | | | | KLTVDKSRWQQGNVFSCSVMHEALHNHYTQ | |
| | | | | KSLSLSPGK | |
| Mesothelin | anetumab | 777 | 778 | QVELVQSGAEVKKPGESLKISCKGSGYSFTSY | DIALTQPASVSGSPGQSITISCTGTSSDIGGY |
| | | | | WIGWVRQAPGKGLEWMGIIDPGDSRTRYSPS | NSVSWYQQHPGKAPKLMIYGVNNRPSGVSN |
| | | | | FQGQVTISADKSISTAYLQWSSLKASDTAMYY | RFSGSKSGNTASLTISGLQAEDEADYYCSSY |
| | | | | CARGQLYGGTYMDGWGQGTLVTVSSASTKG | DIESATPVFGGGTKLTVLGQPKAAPSVTLFPP |
| | | | | PSVFPLAPSSKSTSGGTAALGCLVKDYFPEPV | SSEELQANKATLVCLISDFYPGAVTVAWKGD |
| | | | | TVSWNSGALTSGVHTFPAVLQSSGLYSLSSVV | SSPVKAGVETTTPSKQSNNKYAASSYLSLTP |
| | | | | TVPSSSLGTQTYICNVNHKPSNTKVDKKVEPK | EQWKSHRSYSCQVTHEGSTVEKTVAPTECS |
| | | | | SCDKTHTCPPCPAPELLGGPSVFLFPPKPKDT | |
| | | | | LMISRTPEVTCVVVDVSHEDPEVKFNWYVDG | |
| | | | | VEVHNAKTKPREEQYNSTYRVVSVLTVLHQD | |
| | | | | WLNGKEYKCKVSNKALPAPIEKTISKAKGQPR | |
| | | | | EPQVYTLPPSRDELTKNQVSLTCLVKGFYPSD | |
| | | | | IAVEWESNGQPENNYKTTPPVLDSDGSFFLYS | |
| | | | | KLTVDKSRWQQGNVFSCSVMHEALHNHYTQ | |
| | | | | KSLSLSPGK | |
| MUC1 | clivatuzumab | 779 | 780 | QVQLQQSGAEVKKPGASVKVSCEASGYTFPS | DIQLTQSPSSLSASVGDRVTMTCSASSSVSS |
| | | | | YVLHWVKQAPGQGLEWIGYINPYNDGTQYNE | SYLYWYQQKPGKAPKLWIYSTSNLASGVPA |
| | | | | KFKGKATLTRDTSINTAYMELSRLRSDDTAVY | RFSGSGSGTDFTLTISSLQPEDSASYFCHQW |
| | | | | YCARGFGGSYGFAYWGQGTLVTVSSASTKG | NRYPYTFGGGTRLEIKRTVAAPSVFIFPPSDE |
| | | | | PSVFPLAPSSKSTSGGTAALGCLVKDYFPEPV | QLKSGTASVVCLLNNFYPREAKVQWKVDNAL |
| | | | | TVSWNSGALTSGVHTFPAVLQSSGLYSLSSVV | QSGNSQESVTEQDSKDSTYSLSSTLTLSKAD |
| | | | | TVPSSSLGTQTYICNVNHKPSNTKVDKRVEPK | YEKHKVYACEVTHQGLSSPVTKSFNRGEC |
| | | | | SCDKTHTCPPCPAPELLGGPSVFLFPPKPKDT | |
| | | | | LMISRTPEVTCVVVDVSHEDPEVKFNWYVDG | |
| | | | | VEVHNAKTKPREEQYNSTYRVVSVLTVLHQD | |
| | | | | WLNGKEYKCKVSNKALPAPIEKTISKAKGQPR | |
| | | | | EPQVYTLPPSREEMTKNQVSLTCLVKGFYPSD | |
| | | | | IAVEWESNGQPENNYKTTPPVLDSDGSFFLYS | |
| | | | | KLTVDKSRWQQGNVFSCSVMHEALHNHYTQ | |
| | | | | KSLSLSPGK | |
| MUC1 | gatipotuzumab | 781 | 782 | EVQLVESGGGLVQPGGSMRLSCVASGFPFSN | DIVMTQSPLSNPVTPGEPASISCRSSKSLLHS |
| | | | | YWMNWVRQAPGKGLEWVGEIRLKSNNYTTH | NGITYFFWYLQKPGQSPQLLIYQMSNLASGV |
| | | | | YAESVKGRFTISRDDSKNSLYLQMNSLKTEDT | PDRFSGSGSGTDFTLRISRVEAEDVGVYYCA |
| | | | | AVYYCTRHYYFDYWGQGTLVTVSSASTKGPS | QNLELPPTFGQGTKVEIKRTVAAPSVFIFPPS |
| | | | | VFPLAPSSKSTSGGTAALGCLVKDYFPEPVTV | DEQLKSGTASVVCLLNNFYPREAKVQWKVD |
| | | | | SWNSGALTSGVHTFPAVLQSSGLYSLSSVVTV | NALQSGNSQESVTEQDSKDSTYSLSSTLTLS |
| | | | | PSSSLGTQTYICNVNHKPSNTKVDKKVEPKSC | KADYEKHKVYACEVTHQGLSSPVTKSFNRGE |
| | | | | DKTHTCPPCPAPELLGGPSVFLFPPKPKDTLM | C |
| | | | | ISRTPEVTCVVVDVSHEDPEVKFNWYVDGVE | |
| | | | | VHNAKTKPREEQYNSTYRVVSVLTVLHQDWL | |
| | | | | NGKEYKCKVSNKALPAPIEKTISKAKGQPREP | |
| | | | | QVYTLPPSRDELTKNQVSLTCLVKGFYPSDIA | |
| | | | | VEWESNGQPENNYKTTPPVLDSDGSFFLYSK | |
| | | | | LTVDKSRWQQGNVFSCSVMHEGLHNHYTQK | |
| | | | | SLSLSPGK | |
| Muc5AC | ensituximab | 783 | 784 | QVQLKESGPDLVAPSQSLSITCTVSGFSLSKE | QVVLTQSPVIMSASPGEKVTMTCSASSSISY |
| | | | | GVNWVRQPPGKGLEWLGVIWGDGSTSYNSG | MYWYQQKPGTSPKRWIYDTSKLASGVPARF |
| | | | | LISRLSISKENSKSQVFLKLNSLQADDTATYYC | SGSGSGTSYSLTISNMEAGDAATYYCHQRD |
| | | | | VKPGGDYWGHGTSVTVSSASTKGPSVFPLAP | SYPWTFGGGTNLEIKRTVAAPSVFIFPPSDEQ |
| | | | | SSKSTSGGTAALGCLVKDYFPEPVTVSWNSG | LKSGTASVVCLLNNFYPREAKVQWKVDNALQ |
| | | | | ALTSGVHTFPAVLQSSGPYSLSSVVTVPSSSL | SGNSQESVTEQDSKDSTYSLSSTLTLSKADY |
| | | | | GTQTYICNVNHKPSNTKVDKKVEPKSCDKTHT | EKHKVYACEVTHQGLSSPVTKSFNRGEC |
| | | | | CPPCPAPELLGGPSVFLFPPKPKDTLMISRTP | |
| | | | | EVTCVVVDVSHEDPEVKFNWYVDGVEVHNAK | |
| | | | | TKPREEQYNSTYRVVSVLTVLHQDWLNGKEY | |
| | | | | KCKVSNKALPAPIEKTISKAKGQPREPQVYTLP | |
| | | | | PSRDELTKNQVSLTCLVKGFYPSDIAVEWESN | |
| | | | | GQPENNYKTMPPVLDSDGSFFLYSKLTVDKS | |
| | | | | RWQQGNVFSCSVMHEALHNHYTQKSLSLSP | |
| | | | | GK | |
| Nectin 4 | enfortumab | 785 | 786 | EVQLVESGGGLVQPGGSLRLSCAASGFTFSS | DIQMTQSPSSVSASVGDRVTITCRASQGISG |
| | | | | YNMNWVRQAPGKGLEWVSYISSSSSTIYYAD | WLAWYQQKPGKAPKFLIYAASTLQSGVPSR |
| | | | | SVKGRFTISRDNAKNSLSLQMNSLRDEDTAVY | FSGSGSGTDFTLTISSLQPEDFATYYCQQAN |
| | | | | YCARAYYYGMDVWGQGTTVTVSSASTKGPS | SFPPTFGGGTKVEIKRTVAAPSVFIFPPSDEQ |
| | | | | VFPLAPSSKSTSGGTAALGCLVKDYFPEPVTV | LKSGTASVVCLLNNFYPREAKVQWKVDNALQ |
| | | | | SWNSGALTSGVHTFPAVLQSSGLYSLSSVVTV | SGNSQESVTEQDSKDSTYSLSSTLTLSKADY |
| | | | | PSSSLGTQTYICNVNHKPSNTKVDKRVEPKSC | EKHKVYACEVTHQGLSSPVTKSFNRGEC |
| | | | | DKTHTCPPCPAPELLGGPSVFLFPPKPKDTLM | |
| | | | | ISRTPEVTCVVVDVSHEDPEVKFNWYVDGVE | |
| | | | | VHNAKTKPREEQYNSTYRVVSVLTVLHQDWL | |
| | | | | NGKEYKCKVSNKALPAPIEKTISKAKGQPREP | |
| | | | | QVYTLPPSREEMTKNQVSLTCLVKGFYPSDIA | |
| | | | | VEWESNGQPENNYKTTPPVLDSDGSFFLYSK | |
| | | | | LTVDKSRWQQGNVFSCSVMHEALHNHYTQK | |
| | | | | SLSLSPGK | |
| Notch 1 | brontictuzumab | 787 | 788 | QVQLVQSGAEVKKPGASVKISCKVSGYTLRG | QAVVTQEPSLTVSPGGTVTLTCRSSTGAVTT |
| | | | | YWIEWVRQAPGKGLEWIGQILPGTGRTNYNE | SNYANWFQQKPGQAPRTLIGGTNNRAPGVP |
| | | | | KFKGRVTMTADTSTDTAYMELSSLRSEDTAVY | ARFSGSLLGGKAALTLSGAQPEDEAEYYCA |
| | | | | YCARFDGNYGYYAMDYWGQGTTVTVSSAST | LWYSNHWVFGGGTKLTVLGQPKAAPSVTLF |
| | | | | KGPSVFPLAPCSRSTSESTAALGCLVKDYFPE | PPSSEELQANKATLVCLVSDFYPGAVTVAWK |
| | | | | PVTVSWNSGALTSGVHTFPAVLQSSGLYSLSS | ADGSPVKVGVETTKPSKQSNNKYAASSYLSL |
| | | | | VVTVPSSNFGTQTYTCNVDHKPSNTKVDKTV | TPEQWKSHRSYSCRVTHEGSTVEKTVAPAE |
| | | | | ERKCCVECPPCPAPPVAGPSVFLFPPKPKDTL | CS |
| | | | | MISRTPEVTCVVVDVSHEDPEVQFNWYVDGV | |
| | | | | EVHNAKTKPREEQFNSTFRVVSVLTVVHQDW | |
| | | | | LNGKEYKCKVSNKGLPAPIEKTISKTKGQPRE | |
| | | | | PQVYTLPPSREEMTKNQVSLTCLVKGFYPSDI | |
| | | | | AVEWESNGQPENNYKTTPPMLDSDGSFFLYS | |
| | | | | KLTVDKSRWQQGNVFSCSVMHEALHNHYTQ | |
| | | | | KSLSLSPGK | |
| Notch 2/3 | tarextumab | 789 | 790 | EVQLVESGGGLVQPGGSLRLSCAASGFTFSS | DIVLTQSPATLSLSPGERATLSCRASQSVRS |
| | | | | SGMSWVRQAPGKGLEWVSVIASSGSNTYYA | NYLAWYQQKPGQAPRLLIYGASSRATGVPA |
| | | | | DSVKGRFTISRDNSKNTLYLQMNSLRAEDTAV | RFSGSGSGTDFTLTISSLEPEDFAVYYCQQY |
| | | | | YYCARSIFYTTWGQGTLVTVSSASTKGPSVFP | SNFPITFGQGTKVEIKRTVAAPSVFIFPPSDE |
| | | | | LAPCSRSTSESTAALGCLVKDYFPEPVTVSWN | QLKSGTASVVCLLNNFYPREAKVQWKVDNAL |
| | | | | SGALTSGVHTFPAVLQSSGLYSLSSVVTVPSS | QSGNSQESVTEQDSKDSTYSLSSTLTLSKAD |
| | | | | NFGTQTYTCNVDHKPSNTKVDKTVERKCCVE | YEKHKVYACEVTHQGLSSPVTKSFNRGEC |
| | | | | CPPCPAPPVAGPSVFLFPPKPKDTLMISRTPE | |
| | | | | VTCVVVDVSHEDPEVQFNWYVDGVEVHNAKT | |
| | | | | KPREEQFNSTFRVVSVLTVVHQDWLNGKEYK | |
| | | | | CKVSNKGLPAPIEKTISKTKGQPREPQVYTLPP | |
| | | | | SREEMTKNQVSLTCLVKGFYPSDIAVEWESN | |
| | | | | GQPENNYKTTPPMLDSDGSFFLYSKLTVDKS | |
| | | | | RWQQGNVFSCSVMHEALHNHYTQKSLSLSP | |
| | | | | GK | |
| PD-L1 | atezolizumab | 791 | 792 | EVQLVESGGGLVQPGGSLRLSCAASGFTFSD | DIQMTQSPSSLSASVGDRVTITCRASQDVST |
| | | | | SWIHWVRQAPGKGLEWVAWISPYGGSTYYA | AVAWYQQKPGKAPKLLIYSASFLYSGVPSR |
| | | | | DSVKGRFTISADTSKNTAYLQMNSLRAEDTAV | FSGSGSGTDFTLTISSLQPEDFATYYCQQYL |
| | | | | YYCARRHWPGGFDYWGQGTLVTVSSASTKG | YHPATFGQGTKVEIKRTVAAPSVFIFPPSDEQ |
| | | | | PSVFPLAPSSKSTSGGTAALGCLVKDYFPEPV | LKSGTASVVCLLNNFYPREAKVQWKVDNALQ |
| | | | | TVSWNSGALTSGVHTFPAVLQSSGLYSLSSVV | SGNSQESVTEQDSKDSTYSLSSTLTLSKADY |
| | | | | TVPSSSLGTQTYICNVNHKPSNTKVDKKVEPK | EKHKVYACEVTHQGLSSPVTKSFNRGEC |
| | | | | SCDKTHTCPPCPAPELLGGPSVFLFPPKPKDT | |
| | | | | LMISRTPEVTCVVVDVSHEDPEVKFNWYVDG | |
| | | | | VEVHNAKTKPREEQYASTYRVVSVLTVLHQD | |
| | | | | WLNGKEYKCKVSNKALPAPIEKTISKAKGQPR | |
| | | | | EPQVYTLPPSREEMTKNQVSLTCLVKGFYPSD | |
| | | | | IAVEWESNGQPENNYKTTPPVLDSDGSFFLYS | |
| | | | | KLTVDKSRWQQGNVFSCSVMHEALHNHYTQ | |
| | | | | KSLSLSPGK | |
| PD-L1 | avelumab | 793 | 794 | EVQLLESGGGLVQPGGSLRLSCAASGFTFSS | QSALTQPASVSGSPGQSITISCTGTSSDVGG |
| | | | | YIMMWVRQAPGKGLEWVSSIYPSGGITFYADT | YNYVSWYQQHPGKAPKLMIYDVSNRPSGVS |
| | | | | VKGRFTISRDNSKNTLYLQMNSLRAEDTAVYY | NRFSGSKSGNTASLTISGLQAEDEADYYCSS |
| | | | | CARIKLGTVTTVDYWGQGTLVTVSSASTKGPS | YTSSSTRVFGTGTKVTVLGQPKANPTVTLFP |
| | | | | VFPLAPSSKSTSGGTAALGCLVKDYFPEPVTV | PSSEELQANKATLVCLISDFYPGAVTVAWKA |
| | | | | SWNSGALTSGVHTFPAVLQSSGLYSLSSVVTV | DGSPVKAGVETTKPSKQSNNKYAASSYLSLT |
| | | | | PSSSLGTQTYICNVNHKPSNTKVDKKVEPKSC | PEQWKSHRSYSCQVTHEGSTVEKTVAPTEC |
| | | | | DKTHTCPPCPAPELLGGPSVFLFPPKPKDTLM | S |
| | | | | ISRTPEVTCVVVDVSHEDPEVKFNWYVDGVE | |
| | | | | VHNAKTKPREEQYNSTYRVVSVLTVLHQDWL | |
| | | | | NGKEYKCKVSNKALPAPIEKTISKAKGQPREP | |
| | | | | QVYTLPPSRDELTKNQVSLTCLVKGFYPSDIA | |
| | | | | VEWESNGQPENNYKTTPPVLDSDGSFFLYSK | |
| | | | | LTVDKSRWQQGNVFSCSVMHEALHNHYTQK | |
| | | | | SLSLSPGK | |
| Phosphate- | lifastuzumab | 795 | 796 | EVQLVESGGGLVQPGGSLRLSCAASGFSFSD | DIQMTQSPSSLSASVGDRVTITCRSSETLVHS |
| sodium | | | | FAMSWVRQAPGKGLEWVATIGRVAFHTYYPD | SGNTYLEWYQQKPGKAPKLLIYRVSNRFSG |
| CO- | | | | SMKGRFTISRDNSKNTLYLQMNSLRAEDTAVY | VPSRFSGSGSGTDFTLTISSLQPEDFATYYCF |
| transporter | | | | YCARHRGFDVGHFDFWGQGTLVTVSSASTK | QGSFNPLTFGQGTKVEIKRTVAAPSVFIFPPS |
| | | | | GPSVFPLAPSSKSTSGGTAALGCLVKDYFPEP | DEQLKSGTASVVCLLNNFYPREAKVQWKVD |
| | | | | VTVSWNSGALTSGVHTFPAVLQSSGLYSLSSV | NALQSGNSQESVTEQDSKDSTYSLSSTLTLS |
| | | | | VTVPSSSLGTQTYICNVNHKPSNTKVDKKVEP | KADYEKHKVYACEVTHQGLSSPVTKSFNRGE |
| | | | | KSCDKTHTCPPCPAPELLGGPSVFLFPPKPKD | C |
| | | | | TLMISRTPEVTCVVVDVSHEDPEVKFNWYVD | |
| | | | | GVEVHNAKTKPREEQYNSTYRVVSVLTVLHQ | |
| | | | | DWLNGKEYKCKVSNKALPAPIEKTISKAKGQP | |
| | | | | REPQVYTLPPSREEMTKNQVSLTCLVKGFYPS | |
| | | | | DIAVEWESNGQPENNYKTTPPVLDSDGSFFLY | |
| | | | | SKLTVDKSRWQQGNVFSCSVMHEALHNHYT | |
| | | | | QKSLSLSPGK | |
| PDGF-Rα | olaratumab | 797 | 798 | QLQLQESGPGLVKPSETLSLTCTVSGGSINSS | EIVLTQSPATLSLSPGERATLSCRASQSVSS |
| | | | | SYYWGWLRQSPGKGLEWIGSFFYTGSTYYNP | YLAWYQQKPGQAPRLLIYDASNRATGIPARF |
| | | | | SLRSRLTISVDTSKNQFSLMLSSVTAADTAVYY | SGSGSGTDFTLTISSLEPEDFAVYYCQQRSN |
| | | | | CARQSTYYYGSGNYYGWFDRWDQGTLVTVS | WPPAFGQGTKVEIKRTVAAPSVFIFPPSDEQ |
| | | | | SASTKGPSVFPLAPSSKSTSGGTAALGCLVKD | LKSGTASVVCLLNNFYPREAKVQWKVDNALQ |
| | | | | YFPEPVTVSWNSGALTSGVHTFPAVLQSSGL | SGNSQESVTEQDSKDSTYSLSSTLTLSKADY |
| | | | | YSLSSVVTVPSSSLGTQTYICNVNHKPSNTKV | EKHKVYACEVTHQGLSSPVTKSFNRGEC |
| | | | | DKRVEPKSCDKTHTCPPCPAPELLGGPSVFLF | |
| | | | | PPKPKDTLMISRTPEVTCVVVDVSHEDPEVKF | |
| | | | | NWYVDGVEVHNAKTKPREEQYNSTYRVVSVL | |
| | | | | TVLHQDWLNGKEYKCKVSNKALPAPIEKTISK | |
| | | | | AKGQPREPQVYTLPPSREEMTKNQVSLTCLV | |
| | | | | KGFYPSDIAVEWESNGQPENNYKTTPPVLDS | |
| | | | | DGSFFLYSKLTVDKSRWQQGNVFSCSVMHEA | |
| | | | | LHNHYTQKSLSLSPGK | |
| PDGF-Rα | tovetumab | 799 | 800 | QVQLVESGGGLVKPGGSLRLSCAASGFTFSD | DIQMTQSPSSLSASVGDRVSITCRPSQSFSR |
| | | | | YYMNWIRQAPGKGLEWVSYISSSGSIIYYADS | YINWYQQKPGKAPKLLIHAASSLVGGVPSRF |
| | | | | VKGRFTISRDNAKNSLYLQMNSLRAEDTAVYY | SGSGSGTDFTLTISSLQPEDFATYYCQQTYS |
| | | | | CAREGRIAARGMDVWGQGTTVTVSSASTKGP | NPPITFGQGTRLEMKRTVAAPSVFIFPPSDEQ |
| | | | | SVFPLAPCSRSTSESTAALGCLVKDYFPEPVT | LKSGTASVVCLLNNFYPREAKVQWKVDNALQ |
| | | | | VSWNSGALTSGVHTFPAVLQSSGLYSLSSVVT | SGNSQESVTEQDSKDSTYSLSSTLTLSKADY |
| | | | | VPSSNFGTQTYTCNVDHKPSNTKVDKTVERK | EKHKVYACEVTHQGLSSPVTKSFNRGEC |
| | | | | CCVECPPCPAPPVAGPSVFLFPPKPKDTLMIS | |
| | | | | RTPEVTCVVVDVSHEDPEVQFNWYVDGVEVH | |
| | | | | NAKTKPREEQFNSTFRVVSVLTVVHQDWLNG | |
| | | | | KEYKCKVSNKGLPAPIEKTISKTKGQPREPQV | |
| | | | | YTLPPSREEMTKNQVSLTCLVKGFYPSDIAVE | |
| | | | | WESNGQPENNYKTTPPMLDSDGSFFLYSKLT | |
| | | | | VDKSRWQQGNVFSCSVMHEALHNHYTQKSL | |
| | | | | SLSPGK | |
| PTK7 | cofetuzumab | 801 | 802 | QVQLVQSGPEVKKPGASVKVSCKASGYTFTD | EIVLTQSPATLSLSPGERATLSCRASESVDS |
| | | | | YAVHWVRQAPGKRLEWIGVISTYNDYTYNNQ | YGKSFMHWYQQKPGQAPRLLIYRASNLESG |
| | | | | DFKGRVTMTRDTSASTAYMELSRLRSEDTAV | IPARFSGSGSGTDFTLTISSLEPEDFAVYYCQ |
| | | | | YYCARGNSYFYALDYWGQGTSVTVSSASTKG | QSNEDPWTFGGGTKLEIKRTVAAPSVFIFPP |
| | | | | PSVFPLAPSSKSTSGGTAALGCLVKDYFPEPV | SDEQLKSGTASVVCLLNNFYPREAKVQWKV |
| | | | | TVSWNSGALTSGVHTFPAVLQSSGLYSLSSVV | DNALQSGNSQESVTEQDSKDSTYSLSSTLTL |
| | | | | TVPSSSLGTQTYICNVNHKPSNTKVDKKVEPK | SKADYEKHKVYACEVTHQGLSSPVTKSFNRG |
| | | | | SCDKTHTCPPCPAPELLGGPSVFLFPPKPKDT | EC |
| | | | | LMISRTPEVTCVVVDVSHEDPEVKFNWYVDG | |
| | | | | VEVHNAKTKPREEQYNSTYRVVSVLTVLHQD | |
| | | | | WLNGKEYKCKVSNKALPAPIEKTISKAKGQPR | |
| | | | | EPQVYTLPPSRDELTKNQVSLTCLVKGFYPSD | |
| | | | | IAVEWESNGQPENNYKTTPPVLDSDGSFFLYS | |
| | | | | KLTVDKSRWQQGNVFSCSVMHEALHNHYTQ | |
| | | | | KSLSLSPG | |
| RANKL | denosumab | 803 | 804 | EVQLLESGGGLVQPGGSLRLSCAASGFTFSS | EIVLTQSPGTLSLSPGERATLSCRASQSVRG |
| | | | | YAMSWVRQAPGKGLEWVSGITGSGGSTYYA | RYLAWYQQKPGQAPRLLIYGASSRATGIPD |
| | | | | DSVKGRFTISRDNSKNTLYLQMNSLRAEDTAV | RFSGSGSGTDFTLTISRLEPEDFAVFYCQQY |
| | | | | YYCAKDPGTTVIMSWFDPWGQGTLVTVSSAS | GSSPRTFGQGTKVEIKRTVAAPSVFIFPPSDE |
| | | | | TKGPSVFPLAPCSRSTSESTAALGCLVKDYFP | QLKSGTASVVCLLNNFYPREAKVQWKVDNAL |
| | | | | EPVTVSWNSGALTSGVHTFPAVLQSSGLYSLS | QSGNSQESVTEQDSKDSTYSLSSTLTLSKAD |
| | | | | SVVTVPSSNFGTQTYTCNVDHKPSNTKVDKT | YEKHKVYACEVTHQGLSSPVTKSFNRGEC |
| | | | | VERKCCVECPPCPAPPVAGPSVFLFPPKPKDT | |
| | | | | LMISRTPEVTCVVVDVSHEDPEVQFNWYVDG | |
| | | | | VEVHNAKTKPREEQFNSTFRVVSVLTVVHQD | |
| | | | | WLNGKEYKCKVSNKGLPAPIEKTISKTKGQPR | |
| | | | | EPQVYTLPPSREEMTKNQVSLTCLVKGFYPSD | |
| | | | | IAVEWESNGQPENNYKTTPPMLDSDGSFFLY | |
| | | | | SKLTVDKSRWQQGNVFSCSVMHEALHNHYT | |
| | | | | QKSLSLSPGK | |
| RSPO3 | rosmantuzumab | 805 | 806 | QVQLVQSGAEVKKPGASVKVSCKASGYTFTD | DIQMTQSPSSLSASVGDRVTITCKASQSVDY |
| | | | | YSIHWVRQAPGQGLEWIGYIYPSNGDSGYNQ | DGDSYMNWYQQKPGKAPKLLIYAASNLESG |
| | | | | KFKNRVTMTRDTSTSTAYMELSRLRSEDTAVY | VPSRFSGSGSGTDFTLTISPVQAEDFATYYC |
| | | | | YCATYFANNFDYWGQGTTLTVSSASTKGPSV | QQSNEDPLTFGAGTKLELKRTVAAPSVFIFP |
| | | | | FPLAPSSKSTSGGTAALGCLVKDYFPEPVTVS | PSDEQLKSGTASVVCLLNNFYPREAKVQWKV |
| | | | | WNSGALTSGVHTFPAVLQSSGLYSLSSVVTVP | DNALQSGNSQESVTEQDSKDSTYSLSSTLTL |
| | | | | SSSLGTQTYICNVNHKPSNTKVDKRVEPKSCD | SKADYEKHKVYACEVTHQGLSSPVTKSFNRG |
| | | | | KTHTCPPCPAPELLGGPSVFLFPPKPKDTLMIS | EC |
| | | | | RTPEVTCVVVDVSHEDPEVKFNWYVDGVEVH | |
| | | | | NAKTKPREEQYNSTYRVVSVLTVLHQDWLNG | |
| | | | | KEYKCKVSNKALPAPIEKTISKAKGQPREPQV | |
| | | | | YTLPPSREEMTKNQVSLTCLVKGFYPSDIAVE | |
| | | | | WESNGQPENNYKTTPPVLDSDGSFFLYSKLT | |
| | | | | VDKSRWQQGNVFSCSVMHEALHNHYTQKSL | |
| | | | | SLSPGK | |
| STEAP1 | vandortuzumab | 807 | 808 | EVQLVESGGGLVQPGGSLRLSCAVSGYSITSD | DIQMTQSPSSLSASVGDRVTITCKSSQSLLY |
| | | | | YAWNWVRQAPGKGLEWVGYISNSGSTSYNP | RSNQKNYLAWYQQKPGKAPKLLIYWASTRE |
| | | | | SLKSRFTISRDTSKNTLYLQMNSLRAEDTAVY | SGVPSRFSGSGSGTDFTLTISSLQPEDFATY |
| | | | | YCARERNYDYDDYYYAMDYWGQGTLVTVSS | YCQQYYNYPRTFGQGTKVEIKRTVAAPSVFIF |
| | | | | ASTKGPSVFPLAPSSKSTSGGTAALGCLVKDY | PPSDEQLKSGTASVVCLLNNFYPREAKVQWK |
| | | | | FPEPVTVSWNSGALTSGVHTFPAVLQSSGLY | VDNALQSGNSQESVTEQDSKDSTYSLSSTLT |
| | | | | SLSSVVTVPSSSLGTQTYICNVNHKPSNTKVD | LSKADYEKHKVYACEVTHQGLSSPVTKSFNR |
| | | | | KKVEPKSCDKTHTCPPCPAPELLGGPSVFLFP | GEC |
| | | | | PKPKDTLMISRTPEVTCVVVDVSHEDPEVKFN | |
| | | | | WYVDGVEVHNAKTKPREEQYNSTYRVVSVLT | |
| | | | | VLHQDWLNGKEYKCKVSNKALPAPIEKTISKA | |
| | | | | KGQPREPQVYTLPPSREEMTKNQVSLTCLVK | |
| | | | | GFYPSDIAVEWESNGQPENNYKTTPPVLDSD | |
| | | | | GSFFLYSKLTVDKSRWQQGNVFSCSVMHEAL | |
| | | | | HNHYTQKSLSLSPGK | |
| SLITRK6 | sirtratumab | 809 | 810 | QVQLVESGGGVVQPGRSLRLSCAASGFTFSS | DIVMTQSPLSLPVTPGEPASISCRSSQSLLLS |
| | | | | YGMHWVRQAPGKGLEWVAVIWYDGSNQYYA | HGFNYLDWYLQKPGQSPQLLIYLGSSRASG |
| | | | | DSVKGRFTISRDNSKNTLFLQMHSLRAEDTAV | VPDRFSGSGSGTDFTLKISRVEAEDVGLYYC |
| | | | | YYCARGLTSGRYGMDVWGQGTTVTVSSAST | MQPLQIPWTFGQGTKVEIKRTVAAPSVFIFPP |
| | | | | KGPSVFPLAPCSRSTSESTAALGCLVKDYFPE | SDEQLKSGTASVVCLLNNFYPREAKVQWKV |
| | | | | PVTVSWNSGALTSGVHTFPAVLQSSGLYSLSS | DNALQSGNSQESVTEQDSKDSTYSLSSTLTL |
| | | | | VVTVPSSNFGTQTYTCNVDHKPSNTKVDKTV | SKADYEKHKVYACEVTHQGLSSPVTKSFNRG |
| | | | | ERKCCVECPPCPAPPVAGPSVFLFPPKPKDTL | EC |
| | | | | MISRTPEVTCVVVDVSHEDPEVQFNWYVDGV | |
| | | | | EVHNAKTKPREEQFNSTFRVVSVLTVVHQDW | |
| | | | | LNGKEYKCKVSNKGLPAPIEKTISKTKGQPRE | |
| | | | | PQVYTLPPSREEMTKNQVSLTCLVKGFYPSDI | |
| | | | | AVEWESNGQPENNYKTTPPMLDSDGSFFLYS | |
| | | | | KLTVDKSRWQQGNVFSCSVMHEALHNHYTQ | |
| | | | | KSLSLSPGK | |
| Syndecan | indatuximab | 811 | 812 | QVQLQQSGSELMMPGASVKISCKATGYTFSN | DIQMTQSTSSLSASLGDRVTISCSASQGINNY |
| 1 | | | | YWIEWVKQRPGHGLEWIGEILPGTGRTIYNEK | LNWYQQKPDGTVELLIYYTSTLQSGVPSRFS |
| | | | | FKGKATFTADISSNTVQMQLSSLTSEDSAVYY | GSGSGTDYSLTISNLEPEDIGTYYCQQYSKL |
| | | | | CARRDYYGNFYYAMDYWGQGTSVTVSSAST | PRTFGGGTKLEIKRTVAAPSVFIFPPSDEQLK |
| | | | | KGPSVFPLAPCSRSTSESTAALGCLVKDYFPE | SGTASVVCLLNNFYPREAKVQWKVDNALQS |
| | | | | PVTVSWNSGALTSGVHTFPAVLQSSGLYSLSS | GNSQESVTEQDSKDSTYSLSSTLTLSKADYE |
| | | | | VVTVPSSSLGTKTYTCNVDHKPSNTKVDKRVE | KHKVYACEVTHQGLSSPVTKSFNRGEC |
| | | | | SKYGPPCPSCPAPEFLGGPSVFLFPPKPKDTL | |
| | | | | MISRTPEVTCVVVDVSQEDPEVQFNWYVDGV | |
| | | | | EVHNAKTKPREEQFNSTYRVVSVLTVLHQDW | |
| | | | | LNGKEYKCKVSNKGLPSSIEKTISKAKGQPRE | |
| | | | | PQVYTLPPSQEEMTKNQVSLTCLVKGFYPSDI | |
| | | | | AVEWESNGQPENNYKTTPPVLDSDGSFFLYS | |
| | | | | RLTVDKSRWQEGNVFSCSVMHEALHNHYTQ | |
| | | | | KSLSLSLGK | |
| TRAIL-R2 | conatumumab | 813 | 814 | QVQLQESGPGLVKPSQTLSLTCTVSGGSISSG | EIVLTQSPGTLSLSPGERATLSCRASQGISRS |
| | | | | DYFWSWIRQLPGKGLEWIGHIHNSGTTYYNPS | YLAWYQQKPGQAPSLLIYGASSRATGIPDRF |
| | | | | LKSRVTISVDTSKKQFSLRLSSVTAADTAVYYC | SGSGSGTDFTLTISRLEPEDFAVYYCQQFGS |
| | | | | ARDRGGDYYYGMDVWGQGTTVTVSSASTKG | SPWTFGQGTKVEIKRTVAAPSVFIFPPSDEQL |
| | | | | PSVFPLAPSSKSTSGGTAALGCLVKDYFPEPV | KSGTASVVCLLNNFYPREAKVQWKVDNALQ |
| | | | | TVSWNSGALTSGVHTFPAVLQSSGLYSLSSVV | SGNSQESVTEQDSKDSTYSLSSTLTLSKADY |
| | | | | TVPSSSLGTQTYICNVNHKPSNTKVDKRVEPK | EKHKVYACEVTHQGLSSPVTKSFNRGEC |
| | | | | SCDKTHTCPPCPAPELLGGPSVFLFPPKPKDT | |
| | | | | LMISRTPEVTCVVVDVSHEDPEVKFNWYVDG | |
| | | | | VEVHNAKTKPREEQYNSTYRVVSVLTVLHQD | |
| | | | | WLNGKEYKCKVSNKALPAPIEKTISKAKGQPR | |
| | | | | EPQVYTLPPSREEMTKNQVSLTCLVKGFYPSD | |
| | | | | IAVEWESNGQPENNYKTTPPVLDSDGSFFLYS | |
| | | | | KLTVDKSRWQQGNVFSCSVMHEALHNHYTQ | |
| | | | | KSLSLSPGK | |
| TRAIL-R2 | lexatumumab | 815 | 816 | EVQLVQSGGGVERPGGSLRLSCAASGFTFDD | SSELTQDPAVSVALGQTVRITCQGDSLRSYY |
| | | | | YGMSWVRQAPGKGLEWVSGINWNGGSTGYA | ASWYQQKPGQAPVLVIYGKNNRPSGIPDRF |
| | | | | DSVKGRVTISRDNAKNSLYLQMNSLRAEDTAV | SGSSSGNTASLTITGAQAEDEADYYCNSRDS |
| | | | | YYCAKILGAGRGWYFDLWGKGTTVTVSSAST | SGNHVVFGGGTKLTVLGQPKAAPSVTLFPPS |
| | | | | KGPSVFPLAPSSKSTSGGTAALGCLVKDYFPE | SEELQANKATLVCLISDFYPGAVTVAWKADS |
| | | | | PVTVSWNSGALTSGVHTFPAVLQSSGLYSLSS | SPVKAGVETTTPSKQSNNKYAASSYLSLTPE |
| | | | | VVTVPSSSLGTQTYICNVNHKPSNTKVDKRVE | QWKSHRSYSCQVTHEGSTVEKTVAPTECS |
| | | | | PKSCDKTHTCPPCPAPELLGGPSVFLFPPKPK | |
| | | | | DTLMISRTPEVTCVVVDVSHEDPEVKFNWYVD | |
| | | | | GVEVHNAKTKPREEQYNSTYRVVSVLTVLHQ | |
| | | | | DWLNGKEYKCKVSNKALPAPIEKTISKAKGQP | |
| | | | | REPQVYTLPPSREEMTKNQVSLTCLVKGFYPS | |
| | | | | DIAVEWESNGQPENNYKTTPPVLDSDGSFFLY | |
| | | | | SKLTVDKSRWQQGNVFSCSVMHEALHNHYT | |
| | | | | QKSLSLSPGK | |
| TRAIL-R2 | tigatuzumab | 817 | 818 | EVQLVESGGGLVQPGGSLRLSCAASGFTFSS | DIQMTQSPSSLSASVGDRVTITCKASQDVGT |
| | | | | YVMSWVRQAPGKGLEWVATISSGGSYTYYPD | AVAWYQQKPGKAPKLLIYWASTRHTGVPSR |
| | | | | SVKGRFTISRDNAKNTLYLQMNSLRAEDTAVY | FSGSGSGTDFTLTISSLQPEDFATYYCQQYS |
| | | | | YCARRGDSMITTDYWGQGTLVTVSSASTKGP | SYRTFGQGTKVEIKRTVAAPSVFIFPPSDEQL |
| | | | | SVFPLAPSSKSTSGGTAALGCLVKDYFPEPVT | KSGTASVVCLLNNFYPREAKVQWKVDNALQ |
| | | | | VSWNSGALTSGVHTFPAVLQSSGLYSLSSVVT | SGNSQESVTEQDSKDSTYSLSSTLTLSKADY |
| | | | | VPSSSLGTQTYICNVNHKPSNTKVDKRVEPKS | EKHKVYACEVTHQGLSSPVTKSFNRGEC |
| | | | | CDKTHTCPPCPAPELLGGPSVFLFPPKPKDTL | |
| | | | | MISRTPEVTCVVVDVSHEDPEVKFNWYVDGV | |
| | | | | EVHNAKTKPREEQYNSTYRVVSVLTVLHQDW | |
| | | | | LNGKEYKCKVSNKALPAPIEKTISKAKGQPREP | |
| | | | | QVYTLPPSREEMTKNQVSLTCLVKGFYPSDIA | |
| | | | | VEWESNGQPENNYKTTPPVLDSDGSFFLYSK | |
| | | | | LTVDKSRWQQGNVFSCSVMHEALHNHYTQK | |
| | | | | SLSLSPGK | |
| GPNB | glembatumumab | 819 | 820 | QVQLQESGPGLVKPSQTLSLTCTVSGGSISSE | EIVMTQSPATLSVSPGERATLSCRASQSVDN |
| | | | | NYYWSWIRHHPGKGLEWIGYIYYSGSTYSNP | NLVWYQQKPGQAPRLLIYGASTRATGIPARF |
| | | | | SLKSRVTISVDTSKNQFSLTLSSVTAADTAVYY | SGSGSGTEFTLTISSLQSEDFAVYYCQQYNN |
| | | | | CARGYNWNYFDYWGQGTLVTVSSASTKGPS | WPPWTFGQGTKVEIKRTVAAPSVFIFPPSDE |
| | | | | VFPLAPCSRSTSESTAALGCLVKDYFPEPVTV | QLKSGTASVVCLLNNFYPREAKVQWKVDNAL |
| | | | | SWNSGALTSGVHTFPAVLQSSGLYSLSSVVTV | QSGNSQESVTEQDSKDSTYSLSSTLTLSKAD |
| | | | | PSSNFGTQTYTCNVDHKPSNTKVDKTVERKC | YEKHKVYACEVTHQGLSSPVTKSFNRGEC |
| | | | | CVECPPCPAPPVAGPSVFLFPPKPKDTLMISR | |
| | | | | TPEVTCVVVDVSHEDPEVQFNWYVDGVEVHN | |
| | | | | AKTKPREEQFNSTFRVVSVLTVVHQDWLNGK | |
| | | | | EYKCKVSNKGLPAPIEKTISKTKGQPREPQVY | |
| | | | | TLPPSREEMTKNQVSLTCLVKGFYPSDIAVEW | |
| | | | | ESNGQPENNYKTTPPMLDSDGSFFLYSKLTV | |
| | | | | DKSRWQQGNVFSCSVMHEALHNHYTQKSLS | |
| | | | | LSPGK | |
| Trophoblast | Naptumomab- | 821 | 822 | EVQLQQSGPDLVKPGASVKISCKASGYSFTGY | SIVMTQTPTSLLVSAGDRVTITCKASQSVSND |
| glycoprotein | Fab- | | | YMHWVKQSPGKGLEWIGRINPNNGVTLYNQK | VAWYQQKPGQSPKLLISYTSSRYAGVPDRF |
| | protein | | | FKDKATLTVDKSSTTAYMELRSLTSEDSAVYY | SGSGYGTDFTLTISSVQAEDAAVYFCQQDYN |
| | fusion (type | | | CARSTMITNYVMDYWGQGTSVTVSSAKTTPP | SPPTFGGGTKLEIKRADAAPTVSIFPPSSEQL |
| | 1) | | | SVYPLAPGSAAQTNSMVTLGCLVKGYFPEPVT | TSGGASVVCFLNNFYPKDINVKWKIDGSERQ |
| | | | | VTWNSGSLSSGVHTFPAVLQSDLYTLSSSVTV | NGVLNSWTDQDSKDSTYSMSSTLTLTKDEYE |
| | | | | PSSTWPSETVTCNVAHPASSTKVDKKIVPRDS | RHNSYTCEATHKTSTSPIVKSFNRNES |
| | | | | GGPSEKSEEINEKDLRKKSELQGTALGNLKQI | |
| | | | | YYYNSKAITSSEKSADQFLTNTLLFKGFFTGHP | |
| | | | | WYNDLLVDLGSTAATSEYEGSSVDLYGAYYG | |
| | | | | YQCAGGTPNKTACMYGGVTLHDNNRLTEEKK | |
| | | | | VPINLWIDGKQTTVPIDKVKTSKKEVTVQELDL | |
| | | | | QARHYLHGKFGLYNSDSFGGKVQRGLIVFHS | |
| | | | | SEGSTVSYDLFDAQGQYPDTLLRIYRDNTTISS | |
| | | | | TSLSISLYLYTT | |
| Trop-2 | Sacituzumab | 823 | 824 | QVQLQQSGSELKKPGASVKVSCKASGYTFTN | DIQLTQSPSSLSASVGDRVSITCKASQDVSIA |
| | | | | YGMNWVKQAPGQGLKWMGWINTYTGEPTYT | VAWYQQKPGKAPKLLIYSASYRYTGVPDRF |
| | | | | DDFKGRFAFSLDTSVSTAYLQISSLKADDTAVY | SGSGSGTDFTLTISSLQPEDFAVYYCQQHYIT |
| | | | | FCARGGFGSSYWYFDVWGQGSLVTVSSAST | PLTFGAGTKVEIKRTVAAPSVFIFPPSDEQLK |
| | | | | KGPSVFPLAPSSKSTSGGTAALGCLVKDYFPE | SGTASVVCLLNNFYPREAKVQWKVDNALQS |
| | | | | PVTVSWNSGALTSGVHTFPAVLQSSGLYSLSS | GNSQESVTEQDSKDSTYSLSSTLTLSKADYE |
| | | | | VVTVPSSSLGTQTYICNVNHKPSNTKVDKRVE | KHKVYACEVTHQGLSSPVTKSFNRGEC |
| | | | | PKSCDKTHTCPPCPAPELLGGPSVFLFPPKPK | |
| | | | | DTLMISRTPEVTCVVVDVSHEDPEVKFNWYVD | |
| | | | | GVEVHNAKTKPREEQYNSTYRVVSVLTVLHQ | |
| | | | | DWLNGKEYKCKVSNKALPAPIEKTISKAKGQP | |
| | | | | REPQVYTLPPSREEMTKNQVSLTCLVKGFYPS | |
| | | | | DIAVEWESNGQPENNYKTTPPVLDSDGSFFLY | |
| | | | | SKLTVDKSRWQQGNVFSCSVMHEALHNHYT | |
| | | | | QKSLSLSPGK | |
| TAG-72 | satumomab | 825 | 826 | QVQLQQSDAELVKPGASVKISCKASGYTFTDH | DIQMTQSPASLSVSVGETVTITCRASENIYSN |
| | | | | AIHWAKQKPEQGLEWIGYISPGNDDIKYNEKE | LAWYQQKQGKSPQLLVYAATNLADGVPSRF |
| | | | | KGKATLTADKSSSTAYMQLNSLTSEDSAVYFC | SGSGSGTQYSLKINSLQSEDFGSYYCQHFW |
| | | | | KRSYYGHWGQGTTLTVSSASTKGPSVFPLAP | GTPYTFGGGTRLEIKRADAAPTVFIFPPSDEQ |
| | | | | CSRSTSESTAALGCLVKDYFPEPVTVSWNSG | LKSGTASVVCLLNNFYPREAKVQWKVDNALQ |
| | | | | ALTSGVHTFPAVLQSSGLYSLSSVVTVPSSSL | SGNSQESVTEQDSKDSTYSLSSTLTLSKADY |
| | | | | GTKTYTCNVDHKPSNTKVDKRVCPPCKCPAP | EKHKVYACEVTHQGLSSPVTKSFN |
| | | | | NLLGGPSVFIFPPKIKDVLMISLSPIVTCVVVDV | |
| | | | | SEDDPDVQISWFVNNVEVHTAQTQTHREDYN | |
| | | | | STLRVVSALPIQHQDWMSGKEFKCKVNNKDL | |
| | | | | PAPIERTISKPKGSVRAPQVYVLPPPEEEMTKK | |
| | | | | QVTLTCMVTDFMPEDIYVEWTNNGKTELNYK | |
| | | | | NTEPVLDSDGSYFMYSKLRVEKKNWVERNSY | |
| | | | | SCSVVHEGLHNHHTTKSFSR | |
| TWEAKR | enavatuzumab | 827 | 828 | EVQLVESGGGLVQPGGSLRLSCAASGFTFSS | DIQMTQSPSSLSASVGDRVTITCRASQSVST |
| | | | | YWMSWVRQAPGKGLEWVAEIRLKSDNYATH | SSYSYMHWYQQKPGKAPKLLIKYASNLESG |
| | | | | YAESVKGRFTISRDDSKNSLYLQMNSLRAEDT | VPSRFSGSGSGTDFTLTISSLQPEDFATYYC |
| | | | | AVYYCTGYYADAMDYWGQGTLVTVSSASTKG | QHSWEIPYTFGGGTKVEIKRTVAAPSVFIFPP |
| | | | | PSVFPLAPSSKSTSGGTAALGCLVKDYFPEPV | SDEQLKSGTASVVCLLNNFYPREAKVQWKV |
| | | | | TVSWNSGALTSGVHTFPAVLQSSGLYSLSSVV | DNALQSGNSQESVTEQDSKDSTYSLSSTLTL |
| | | | | TVPSSSLGTQTYICNVNHKPSNTKVDKKVEPK | SKADYEKHKVYACEVTHQGLSSPVTKSFNRG |
| | | | | SCDKTHTCPPCPAPELLGGPSVFLFPPKPKDT | EC |
| | | | | LMISRTPEVTCVVVDVSHEDPEVKFNWYVDG | |
| | | | | VEVHNAKTKPREEQYNSTYRVVSVLTVLHQD | |
| | | | | WLNGKEYKCKVSNKALPAPIEKTISKAKGQPR | |
| | | | | EPQVYTLPPSREEMTKNQVSLTCLVKGFYPSD | |
| | | | | IAVEWESNGQPENNYKTTPPVLDSDGSFFLYS | |
| | | | | KLTVDKSRWQQGNVFSCSVMHEALHNHYTQ | |
| | | | | KSLSLSPGK | |
| TYRP1 | flanvotumab | 829 | 830 | QVQLVQSGSELKKPGASVKISCKASGYTFTSY | EIVLTQSPATLSLSPGERATLSCRASQSVSS |
| | | | | AMNWVRQAPGQGLESMGWINTNTGNPTYAQ | YLAWYQQKPGQAPRLLIYDASNRATGIPARF |
| | | | | GFTGRFVFSMDTSVSTAYLQISSLKAEDTAIYY | SGSGSGTDFTLTISSLEPEDFAVYYCQQRSN |
| | | | | CAPRYSSSWYLDYWGQGTLVTVSSASTKGPS | WLMYTFGQGTKLEIKRTVAAPSVFIFPPSDE |
| | | | | VFPLAPSSKSTSGGTAALGCLVKDYFPEPVTV | QLKSGTASVVCLLNNFYPREAKVQWKVDNAL |
| | | | | SWNSGALTSGVHTFPAVLQSSGLYSLSSVVTV | QSGNSQESVTEQDSKDSTYSLSSTLTLSKAD |
| | | | | PSSSLGTQTYICNVNHKPSNTKVDKRVEPKSC | YEKHKVYACEVTHQGLSSPVTKSFNRGEC |
| | | | | DKTHTCPPCPAPELLGGPSVFLFPPKPKDTLM | |
| | | | | ISRTPEVTCVVVDVSHEDPEVKFNWYVDGVE | |
| | | | | VHNAKTKPREEQYNSTYRVVSVLTVLHQDWL | |
| | | | | NGKEYKCKVSNKALPAPIEKTISKAKGQPREP | |
| | | | | QVYTLPPSREEMTKNQVSLTCLVKGFYPSDIA | |
| | | | | VEWESNGQPENNYKTTPPVLDSDGSFFLYSK | |
| | | | | LTVDKSRWQQGNVFSCSVMHEALHNHYTQK | |
| | | | | SLSLSPGK | |
| Tyrosine- | zilovertamab | 831 | 832 | QVQLQESGPGLVKPSQTLSLTCTVSGYAFTAY | DIVMTQTPLSLPVTPGEPASISCRASKSISKY |
| protein | | | | NIHWVRQAPGQGLEWMGSFDPYDGGSSYNQ | LAWYQQKPGQAPRLLIYSGSTLQSGIPPRFS |
| kinase | | | | KFKDRLTISKDTSKNQVVLTMTNMDPVDTATY | GSGYGTDFTLTINNIESEDAAYYFCQQHDES |
| transmembrane | | | | YCARGWYYFDYWGHGTLVTVSSASTKGPSVF | PYTFGEGTKVEIKRTVAAPSVFIFPPSDEQLK |
| receptor | | | | PLAPSSKSTSGGTAALGCLVKDYFPEPVTVSW | SGTASVVCLLNNFYPREAKVQWKVDNALQS |
| | | | | NSGALTSGVHTFPAVLQSSGLYSLSSVVTVPS | GNSQESVTEQDSKDSTYSLSSTLTLSKADYE |
| | | | | SSLGTQTYICNVNHKPSNTKVDKKVEPKSCDK | KHKVYACEVTHQGLSSPVTKSFNRGEC |
| | | | | THTCPPCPAPELLGGPSVFLFPPKPKDTLMIS | |
| | | | | RTPEVTCVVVDVSHEDPEVKFNWYVDGVEVH | |
| | | | | NAKTKPREEQYNSTYRVVSVLTVLHQDWLNG | |
| | | | | KEYKCKVSNKALPAPIEKTISKAKGQPREPQV | |
| | | | | YTLPPSRDELTKNQVSLTCLVKGFYPSDIAVE | |
| | | | | WESNGQPENNYKTTPPVLDSDGSFFLYSKLT | |
| | | | | VDKSRWQQGNVFSCSVMHEALHNHYTQKSL | |
| | | | | SLSPGK | |
| Vimentin | pritumumab | 833 | 834 | EVQLLESGGDLVQPGGSLRLSCAASGFTFSN | DIQMTQSPSSLSASVGDRVTITCRASQDISNY |
| | | | | YAMSWVRQAPGKGLEWVSAITPSGGSTNYAD | LAWFQQKPGKAPKSLIYAASSLHSKVPTQF |
| | | | | SVKGRFTISRDNSQNTLYLQMNSLRVEDTAVY | SGSGSGTDFTLTISSLQPEDFATYYCLQYST |
| | | | | ICGRVPYRSTWYPLYWGQGTLVTVSSASTKG | YPITFGGGTKVEIKRTVAAPSVFIFPPSDEQLK |
| | | | | PSVFPLAPSSKSTSGGTAALGCLVKDYFPEPV | SGTASVVCLLNNFYPREAKVQWKVDNALQS |
| | | | | TVSWNSGALTSGVHTFPAVLQSSGLYSLSSVV | GNSQESVTEQDSKDSTYSLSSTLTLSKADYE |
| | | | | TVPSSSLGTQTYICNVNHKPSNTKVDKKVEPK | KHKVYACEVTHQGLSSPVTKSFNRGEC |
| | | | | SCDKTHTCPPCPAPELLGGPSVFLFPPKPKDT | |
| | | | | LMISRTPEVTCVVVDVSHEDPEVKFNWYVDG | |
| | | | | VEVHNAKTKPREEQYNSTYRVVSVLTVLHQD | |
| | | | | WLNGKEYKCKVSNKALPAPIEKTISKAKGQPR | |
| | | | | EPQVYTLPPSRDELTKNQVSLTCLVKGFYPSD | |
| | | | | IAVEWESNGQPENNYKTTPPVLDSDGSFFLYS | |
| | | | | KLTVDKSRWQQGNVFSCSVMHEALHNHYTQ | |
| | | | | KSLSLSPGK | |

Similarly, monoclonal antibodies that bind surface markers for many other cell types are known. When treating fibrosis, the conditioning agent can be targeted to cells-expressing FAP, Thy1 (CD90), PDGF-Rα, or DDR2. Examples of anti-FAP antibodies include sibrotuzumab and 4G5, as disclosed above. Examples of anti-PDGF-Ra antibodies include olaratumab and tovetumab, as disclosed above. Numerous reagent monoclonal antibodies recognizing Thy1 (CD90), or DDR2 are commercially available, but would ideally be humanized for use in the disclosed methods.

When treating antibody-mediated autoimmunity the conditioning agent can be targeted to B lineage cells. B cell lineage markers include BCMA, CD19, CD20, CD22, CD37, CD38, CD74, CD79B, CD80, CD138, CD319, GPRC5D, RANKL, and TRAIL-R1. Examples of monoclonal antibodies binding these markers are disclosed in the list of anti-tumor antigen antibodies above.

Monoclonal antibodies can be adapted to use as binding moieties in a variety of ways. The whole antibody, whether commercially available or isolated from hybridoma (or other cell) culture, can itself be used as a binding moiety or cleaved to generate Fab or F(ab)2 and the fragment used as the binding moiety. Accordingly, it is not necessary to know the amino acid sequence of the antibody. Production of engineered forms of antibody that can be used as a binding moiety, such as scFv, minibodies, and diabodies, is facilitated by knowledge of the amino acid sequence and/or access to the cloned genes. It is within the skill of one in the art to clone and sequence antibody encoding genes from hybridomas (or other cells expressing the mAb). The amino acid sequences of many antibodies are published and can be found in databases such as The ABCD (AntiBodies Chemically Defined) Database (web.expasy.org/abcd/), the international ImMunoGeneTics information system monoclonal antibody database (https://www.imgt.org/mAb-DB/), and the Therapeutic Antibody Database (https://tabs.craic.com/), as well as in Dataset S2 of Jain et al., Proc. Natl. Acad. Sci. 114(5):944-949 (2017) and through Table 9 of U.S. Pat. No. 11,326,182, both of which are incorporated by reference.

When treating T cell-mediated autoimmunity the conditioning agent can be targeted to CD4⁺ cells, especially Th17 cells which are predominantly CD4⁺. Examples of anti-CD4 antibodies include OKT4, keliximab (IDEC CE9.1), MTRX1011A, Ibalizumab (TNX-355, Hu5A8), and RPA-T4. Nanoparticles targeted to CD4 are disclosed in WO2022081702 which is incorporated by reference for all that it teaches regarding the manufacture and use of CD4-targeted nanoparticles that is not inconsistent with the present disclosure. The Th17 subset plays a major role in T cell-mediated autoimmunity. This subset can be targeted by binding to CCR4 or CCR6. Examples of CCR4 binding monoclonal antibodies include mogamulizumab (humanized), mAb2-3 (humanized 1567), and KM2760 (chimeric). Further mouse monoclonal antibodies recognizing CCR4 are disclosed in U.S. Pat. No. 6,488,930 which is incorporated by reference herein in its entirety to the extent that it is not inconsistent with the present disclosure. Examples of CCR6 binding monoclonal antibodies include KM4703, BV786, and 18B9E6. Additionally, human antibodies recognizing CCR6 are disclosed in WO2013184218A1 which is incorporated by reference herein in its entirety to the extent that it is not inconsistent with the present disclosure.

Although the herein disclosed conditioning regimens are generally agnostic to the specificity of the reprogramming agent these various monoclonal antibodies can also be useful to provide the specificity of a reprogramming agent, whether that is a CAR or a BiTE. When the reprogramming agent is a TCR epitopes from internal antigens, such as the tumor antigens MAGE, NY-ESO, human papilloma virus E6 and E7 proteins (found in cervical cancer (and also oropharyngeal, anal, penile, vaginal and vulvar cancers)), and many others known in the literature, can additionally be the target of the reprogramming agent.

The tLNP further comprises a nucleic acid. In various embodiments the nucleic acid is mRNA, self-replicating RNA, siRNA, miRNA, antisense oligonucleotides, DNA, DNA-RNA hybrids, a gene editing component (for example, a guide RNA a tracr RNA, sgRNA, an mRNA encoding an RNA-guided nuclease, a gene or base editing protein, a zinc-finger nuclease, a Talen, a CRISPR nuclease, such as Cas9, Cas12 or CasX, a DNA molecule to be inserted or serve as a template for repair), and the like, or a combination thereof. In some embodiments the mRNA encodes a chimeric antigen receptor (CAR). In some embodiments the mRNA encodes a TCR. In some embodiments, the mRNA encodes a bispecific T cell engager. In other embodiments, the mRNA encodes a gene-editing or base-editing protein. In some embodiments, the nucleic acid is a guide RNA. In some embodiments, the LNP or tLNP comprises both a gene- or base-editing protein-encoding mRNA and one or more guide RNAs. CRISPR nucleases may have altered activity, for example, modifying the nuclease so that it is a nickase instead of making double-strand cuts or so that it binds the sequence specified by the guide RNA but has no enzymatic activity. Base-editing proteins are often fusion proteins comprising a deaminase domain and a sequence-specific DNA binding domain (such as an inactive CRISPR nuclease). In alternative embodiments, rather than comprising an mRNA encoding an RNA-guided nuclease and a guide RNA, the nanoparticle comprises a ribonucleoprotein, that is a complex comprising a guide RNA bound to an RNA-guided nuclease. In other embodiments, the nanoparticle comprises an RNA and reverse transcriptase. In still other embodiments, the nanoparticle comprises a virion, virus-like particle, or nucleocapsid. Genome-, gene-, and base-editing technology are reviewed in Anzalone et al., Nature Biotechnology 38:824-844, 2020, Sakuma, Gene and Genome Editing 3-4:100017, 2022, and Zhou et al., MedComm 3(3):e155, 2022, each of which is incorporated by reference for all that they teach about the components and uses of this technology to the extent that it does not conflict with the present disclosure.

In some embodiments, the RNA comprises at least one modified nucleoside. In some embodiments, the modified nucleoside is pseudouridine, N1-methylpseudouridine, 5-methylcytosine, 5-methyluridine, N6-methyladenosine, 2′-O-methyluridine, or 2-thiouridine.

With respect to the LNP or the tLNP, in some embodiments the ratio of total lipid to nucleic acid is 10:1 to 50:1 on a weight basis. In some embodiments, that ratio of total lipid to nucleic acid is 10:1, 20:1, 30:1, or 40:1 to 50:1, or 10:1 to 20:1, 30:1, 40:1 or 50:1, or any range bound by a pair of these ratios.

With respect to the LNP or the tLNP, in some embodiments the ratio of positively-chargeable lipid amine (N=nitrogen) groups to negatively-charged nucleic acid phosphate (P) groups (N/P) is in the range of 4 to 8. In some instances, N/P is 6.

With respect to the various nanoparticle (including tLNP and tropic LNP) embodiments, some embodiments specifically exclude one or more of the of the various, embodiments, instances, or species of lipid or nucleic acid. Some embodiments specifically exclude various ionizable cationic lipids, phospholipids, sterols, co-lipids, PEG-lipids and/or functionalized PEG-lipids. Other embodiments specifically include such features.

Certain aspects include an activating conditioning regimen for expanding the number of polyfunctional immune effector cells or mobilizing immune effector cells comprising providing an activating conditioning agent prior to or concurrently with an in vivo engineering agent, wherein the activating conditioning agent comprises a γ-chain receptor cytokine or other agonist, an inflammatory chemokine, a pan-activating cytokine or a CTLA-4 checkpoint inhibitor. In some embodiments, the activating conditioning agent is provided by administering the activating conditioning agent. In some embodiments, the activating conditioning agent is provided by administering a nanoparticle comprising a nucleic acid encoding the activating conditioning agent. In some embodiments the activating conditioning agent or its encoding nucleic acid are referred to as means for activating conditioning. Some embodiments specifically include or exclude one or more species of activating conditioning agent. In various embodiments, the activating conditioning agents are applied as disclosed herein for the particular agent.

Certain aspects include an adjuvant conditioning regimen for diminishing Treg cell activity or recruiting endogenous immunity comprising providing an adjuvant conditioning agent concurrently with or after an in vivo engineering agent, wherein the adjuvant conditioning agent comprises an immune checkpoint inhibitor, low-dose cyclophosphamide, a γ-chain receptor cytokine or other agonist, an antigen presenting cell activity enhancer, or a pan-activating cytokine. In some embodiments, the adjuvant conditioning agent is provided by administering the adjuvant conditioning agent. In some embodiments, the adjuvant conditioning agent is provided by administering a nanoparticle comprising a nucleic acid encoding the adjuvant conditioning agent. In some embodiments the adjuvant conditioning agent or its encoding nucleic acid are referred to as means for adjuvant conditioning. Some embodiments specifically include or exclude one or more species of adjuvant conditioning agent. In various embodiments, the adjuvant conditioning agents are applied as disclosed herein for the particular agent.

Certain aspects include a composition comprising a targeted nanoparticle bearing a binding moiety on its surface to target the nanoparticle to a tumor or other diseased tissue and comprising a biological response modifier or a nucleic acid encoding the biological response modifier. In some embodiments, the nanoparticle is a lipid nanoparticle. In some embodiments, the binding moiety comprises an antibody or antigen binding portion thereof. In some embodiments, the binding moiety binds to a tumor antigen expressed on the surface of a tumor cell. In some instances, the tumor cell is a neoplastic cell. In some instances, the tumor cell is a stromal cell. In some embodiments, the disease is a B cell-mediated autoimmunity, and the binding moiety binds a B cell lineage marker. In some embodiments, the disease is a T cell-mediated autoimmunity, and the binding moiety binds CD4, CCR4, or CCR6. In some embodiments, the disease is a fibrotic condition and the binding moiety binds a fibrogenic cell marker such as FAP or periostin.

In various embodiments, the BRM comprises a γ-chain receptor cytokine, an immune checkpoint inhibitor, an inflammatory chemokine, an enhancer of APC activity, or a highly active cytokine, for example, as disclosed herein.

The following examples are intended to illustrate various embodiments of the invention. As such, the specific embodiments discussed are not to be constructed as limitations on the scope of the invention. It will be apparent to one skilled in the art that various equivalents, changes, and modifications may be made without departing from the scope of invention, and it is understood that such equivalent embodiments are to be included herein.

### Administration of Conditioning Agents

The present technology includes methods of conditioning a subject who receives an immune cell in vivo engineering agent comprising providing a conditioning agent by systemic administration to the subject prior to, during, or after administration of the immune cell in vivo engineering agent. In alternative embodiments, local administration, such as intralesional, topical, or intraperitoneal administrations, can be used.

In some embodiments, the present technology includes a method of conditioning a subject who receives an immune cell in vivo engineering agent comprising providing a conditioning agent, wherein the conditioning agent comprises a γ-chain receptor cytokine or other γ-chain receptor agonist, to the subject prior to administration of the immune cell in vivo engineering agent, wherein the γ-chain receptor cytokine is provided by systemic administration of the cytokine or other γ-chain receptor agonist. In some aspects, the γ-chain receptor cytokine comprises IL-15, IL-2, IL-7, or IL-21.

In some aspects, the systemic administration of the cytokine is by intravenous or subcutaneous infusion or injection. In some aspects, the γ-chain receptor cytokine is provided to the subject by 3 weekly administrations and the third administration is 3 to 7 days before the subject receives the immune cell in vivo engineering agent.

In some aspects, conditioning increases the number of polyfunctional immune effector cells. In some aspects, conditioning leads to mobilization of reprogrammed cells into a tumor or other locus of disease subsequent to administration of the immune cell in vivo engineering agent.

In some aspects, the systemic administration of the immune checkpoint inhibitor is by intravenous or subcutaneous infusion or injection. In some aspects, the systemic administration of the immune checkpoint inhibitor occurs at 3-week intervals. In some aspects, the first administration of the immune cell in vivo engineering agent occurs about 1 week after a 2^{nd} systemic administration of the immune checkpoint inhibitor.

In some aspects, the immune checkpoint inhibitor is an anti-CTLA-4 antibody. In some aspects, the immune checkpoint inhibitor is an anti-PD-1, anti-PD-L1, anti-Tim-3, or anti-LAG-3 antibody.

In some aspects, conditioning reduces Treg cell activity. In some aspects, conditioning activates T effector cells. In some aspects, conditioning mobilizes immune cells into a tumor or other locus of disease.

In some embodiments, the present technology includes a method of conditioning a subject who receives an immune cell in vivo engineering agent comprising providing a conditioning agent, wherein the conditioning agent comprises an agent that enhances the activity of antigen presenting cells, to the subject prior to, concurrently with, or subsequent to administration of the immune cell in vivo engineering agent. In some embodiments, the agent that enhances the activity of antigen presenting cells is provided by systemic administration of the agent. In some embodiments, the administration of the agent that enhances the activity of antigen presenting cells is by intravenous, intralesional, or intraperitoneal infusion or injection.

In some aspects, the agent that enhances the activity of antigen presenting cells is provided 3-4 days and 12-24 hours prior to the in vivo immune cell engineering agent. In some aspects, the agent that enhances the activity of antigen presenting cells is provided anytime the same day as or 12-24 hours in advance of each of multiple administrations of the in vivo immune cell engineering agent. In some aspects, the agent that enhances the activity of antigen presenting cells is provided every 3-7 days subsequent to a pause in or conclusion of treatment with the in vivo immune cell engineering agent while the tumor is shrinking.

In some aspects, the agent that enhances the activity of antigen presenting cells comprises Flt-3 ligand, gm-CSF, or IL-18.

In some aspects, epitope spreading is promoted by the conditioning. In some aspects, polyfunctional effector cells are expanded.

In some embodiments, the present technology includes a method of conditioning a subject who receives an immune cell in vivo engineering agent comprising administering low-dose cyclophosphamide prior to administration of the immune cell in vivo engineering agent.

In some aspects, the cyclophosphamide is administered with metronomic dosing. In some aspects, the cyclophosphamide is administered at a dose of 50 mg daily or 100 mg every other day. In some aspects, the cyclophosphamide is administered over a period of 5 to 8 days. In some aspects, the cyclophosphamide is administered at a daily dose of 10-50 mg for up to 3 days. In some aspects, the immune cell in vivo engineering agent is administered 3 to 4 days after a last dose of the cyclophosphamide. In some aspects, Treg cell activity is reduced.

In some embodiments, the present technology includes a method of activating conditioning for a subject who receives an immune cell in vivo engineering agent comprising providing an activating conditioning agent prior to or concurrently with the in vivo engineering agent, wherein the activating conditioning agent comprises a γ-chain receptor cytokine, an inflammatory chemokine, a pan-activating cytokine, an antigen presenting cell activity enhancer, or a CTLA-4 checkpoint inhibitor.

In some embodiments, the present technology includes a method of adjuvant conditioning for a subject who receives an immune cell in vivo engineering agent comprising providing an adjuvant conditioning agent concurrently with or after an in vivo engineering agent, wherein the adjuvant conditioning agent comprises an immune checkpoint inhibitor, low-dose cyclophosphamide, a γ-chain receptor cytokine, an antigen presenting cell activity enhancer, an anti-CCR4 antibody, or a pan-activating cytokine. In some aspects, Treg cell activity is reduced, or endogenous immunity is recruited to a tumor or other locus of disease.

In some aspects, the activating or adjuvant conditioning agent is provided as a nanoparticle comprising a nucleic acid encoding the activating or adjuvant conditioning agent.

In some embodiments, the immune cell in vivo engineering agent comprises a nucleic acid encoding a reprogramming agent packaged in a nanoparticle, wherein the reprogramming agent is a chimeric antigen receptor (CAR), an T cell receptor (TCR), or an immune cell or T cell engager (such as a BiTE). In some aspects, the nucleic acid encoding the reprogramming agent is an mRNA. In some aspects, the nanoparticle in which the nucleic acid encoding a reprogramming agent is packaged is a tropic lipid nanoparticle. In some aspects, the nanoparticle in which the nucleic acid encoding a reprogramming agent is packaged is targeted nanoparticle (tNP). In some aspects, the tNP is a targeted lipid nanoparticle (tLNP). In some aspects, the targeted nanoparticle comprises a binding moiety on its surface. In some aspects, the binding moiety comprises an antibody antigen binding domain. In some aspects, the binding moiety binds to a T cell or NK cell surface antigen. In some aspects, the nanoparticle is a tLNP comprising a binding moiety on its surface, wherein the binding moiety binds to a tumor surface antigen. In some aspects, the binding moiety comprises means for binding an immune cell. In some aspects, the binding moiety comprises means for binding CD5.

Targeted Administration of mRNA Encoding a BRM as a Conditioning Agent

As with administration of the BRM itself, administration of tLNP comprising a nucleic acid encoded BRM can be administered systemically, for example by intravenous or subcutaneous injection or infusion, or locally, such as by intralesional, topical, or intraperitoneal administrations.

In some embodiments, the present technology includes a method of conditioning a subject who receives an immune cell in vivo engineering agent comprising providing a conditioning agent, wherein the conditioning agent comprises a γ-chain receptor cytokine or other γ-chain receptor agonist, to the subject prior to administration of the immune cell in vivo engineering agent, wherein the γ-chain receptor cytokine is provided by administration of a nanoparticle comprising a nucleic acid encoding the γ-chain receptor cytokine. In some aspects, the γ-chain receptor cytokine comprises IL-15, IL-2, IL-7, or IL-21.

In some aspects the administration of a nanoparticle comprising a nucleic acid encoding the γ-chain receptor cytokine, is by intravenous or subcutaneous infusion or injection. In some aspects, the nanoparticle is provided to the subject by 3 weekly administrations and the third administration is 3 to 7 days before the subject receives the immune cell in vivo engineering agent.

In some aspects, conditioning increases the number of polyfunctional immune effector cells. In some aspects, conditioning leads to mobilization of reprogrammed cells into a tumor or other locus of disease subsequent to administration of the immune cell in vivo engineering agent.

In some embodiments, the present technology includes a method of conditioning a subject who receives an immune cell in vivo engineering agent comprising providing a conditioning agent, wherein the conditioning agent comprises an immune checkpoint inhibitor, to the subject prior to, concurrently with, or subsequent to administration of the immune cell in vivo engineering agent, wherein the immune checkpoint inhibitor is provided by administration of a nanoparticle comprising a nucleic acid encoding the immune checkpoint inhibitor. In some aspects, the immune checkpoint inhibitor is an anti-CTLA-4 antibody. In some aspects, the immune checkpoint inhibitor is an anti-PD-1, anti-PD-L1, anti-Tim-3, or anti-LAG-3 antibody.

In some aspects, the administration of the nanoparticle comprising the nucleic acid encoding the immune checkpoint inhibitor is by intravenous or subcutaneous infusion or injection. In some aspects, administration of the nanoparticle comprising the nucleic acid encoding the immune checkpoint inhibitor occurs every 3 to 7 days over a period of 1 week to 1 month. In some aspects, a first administration of the immune cell in vivo engineering agent occurs at least about 2 weeks after a first administration of the nanoparticle comprising the nucleic acid encoding the immune checkpoint inhibitor.

In some aspects, conditioning reduces Treg cell activity. In some aspects, conditioning activates T effector cells. In some aspects, conditioning mobilizes immune cells into a tumor or other locus of disease.

In some embodiments, the present technology includes a method of conditioning a subject who receives an immune cell in vivo engineering agent comprising administering a conditioning agent, wherein the conditioning agent comprises a nanoparticle comprising a nucleic acid encoding an inflammatory chemokine. In some aspects, the inflammatory chemokine comprises CCL2, CCL3, CCL4, CCL5, CCL11, CXCL1, CXCL2, CXCL-8, CXCL9, CXCL10, or CXCL11. In some aspects, the inflammatory chemokine comprises CCL5.

In some aspects, the administration of the nanoparticle comprising the nucleic acid encoding the inflammatory chemokine is by intravenous, intralesional, or intraperitoneal infusion or injection. In some aspects, the nanoparticle comprising the nucleic acid encoding the inflammatory chemokine is administered at 3- to 4-day intervals. In some aspects, the nanoparticle comprising the nucleic acid encoding the inflammatory chemokine is administered 2, 3, or 4 times prior to administration of the immune cell in vivo engineering agent. In some aspects, the immune cell in vivo engineering agent is administered the day following the most recent administration of the nanoparticle comprising the nucleic acid encoding the inflammatory chemokine. In some aspects, the nanoparticle comprising the nucleic acid encoding the inflammatory chemokine is administered following every 1, 2, or 3 administrations of the in vivo engineering agent. In some aspects, the conditioning expands and/or mobilizes immune cells to a tumor or other locus of disease.

In some embodiments, the present technology includes a method of conditioning a subject who receives an immune cell in vivo engineering agent comprising providing a conditioning agent, wherein the conditioning agent comprises an agent that enhances the activity of antigen presenting cells, to the subject prior to, concurrently with, or subsequent to administration of the immune cell in vivo engineering agent, wherein the agent that enhances the activity of antigen presenting cells is provided by administration of a nanoparticle comprising a nucleic acid encoding the agent that enhances the activity of antigen presenting cells. In some aspects, the agent that enhances the activity of antigen presenting cells comprises Flt-3 ligand, gm-CSF, or IL-18.

In some aspects, the administration of a nanoparticle comprising a nucleic acid encoding the agent that enhances the activity of antigen presenting cells, is by intravenous, intralesional, or intraperitoneal infusion or injection. In some aspect, the agent that enhances the activity of antigen presenting cells is provided 3-4 days and 12-24 hours prior to the in vivo immune cell engineering agent. In some aspects, the agent that enhances the activity of antigen presenting cells is provided anytime the same day as or 12-24 hours in advance of each of multiple administrations of the in vivo immune cell engineering agent. In some aspects, the agent that enhances the activity of antigen presenting cells is provided every 3-7 days subsequent to a pause in or conclusion of treatment with the in vivo immune cell engineering agent while the tumor is shrinking.

In some aspects, the conditioning promotes epitope spreading. In some aspects, the conditioning expands polyfunctional effector cells.

In some embodiments, the present technology includes a method of conditioning a subject who receives an immune cell in vivo engineering agent comprising administering a conditioning agent, wherein the conditioning agent comprises a nanoparticle comprising a nucleic acid encoding a pan-activating cytokine, prior or subsequent to administration of the immune cell in vivo engineering agent. In some aspects, the pan-activating cytokine comprises IL-12 or IL-18.

In some embodiments, the present technology includes a method of activating conditioning for a subject who receives an immune cell in vivo engineering agent comprising providing an activating conditioning agent prior to or concurrently with the in vivo engineering agent, wherein the activating conditioning agent comprises a nanoparticle comprising a nucleic acid encoding a γ-chain receptor cytokine, an inflammatory chemokine, a pan-activating cytokine, an antigen presenting cell activity enhancer, or a CTLA-4 checkpoint inhibitor.

In some embodiments, the present technology includes a method of adjuvant conditioning for a subject who receives an immune cell in vivo engineering agent comprising providing an adjuvant conditioning agent concurrently with or after an in vivo engineering agent, wherein the adjuvant conditioning agent comprises a nanoparticle comprising a nucleic acid encoding an immune checkpoint inhibitor, low-dose cyclophosphamide, a γ-chain receptor cytokine, an antigen presenting cell activity enhancer, an anti-CCR4 antibody, or a pan-activating cytokine. In some aspects, Treg cell activity is reduced, or endogenous immunity is recruited to a tumor or other locus of disease.

In some aspects, the administration of the nanoparticle comprising the nucleic acid encoding the pan-activating cytokine is by intravenous, intralesional, or intraperitoneal infusion or injection. In some aspects, the nanoparticle comprising the nucleic acid encoding the pan-activating cytokine is administered at 3- to 4-day intervals. In some aspects, the nanoparticle comprising the nucleic acid encoding the pan-activating cytokine is administered 1, 2, 3, or 4 times prior to administration of the immune cell in vivo engineering agent which is administered 1 to 7 days after the most recent administration of the nucleic acid encoding the pan-activating cytokine. In some aspects, the nanoparticle comprising the nucleic acid encoding the pan-activating cytokine is administered within 4 days following the most recent administration of the immune cell in vivo engineering agent. In some aspects, the conditioning activates immune cells in a tumor or other locus of disease.

In some embodiments, the nanoparticle in which the conditioning agent is provided is a targeted nanoparticle. In some aspects, the targeted nanoparticle comprises a binding moiety on its surface. In some aspects, the binding moiety comprises an antibody antigen binding domain. In some aspects, the binding moiety comprises means for binding an immune cell. In some aspects, the binding moiety comprises means for binding CD5. In some aspects, the binding moiety binds to a tumor surface antigen. In some aspects, the binding moiety comprises means for binding a tumor surface antigen.

In some aspects, the nanoparticle is a lipid nanoparticle. In some aspects, the nanoparticle in which the conditioning agent is provided is a tropic lipid nanoparticle. In some aspects, the nucleic acid encoding the conditioning agent is an mRNA.

Many of the herein disclosed aspects are methods of conditioning a subject who receives an engineering agent wherein the method does not include a positive step of administering the engineering agent. For each such aspects there is a parallel aspect further comprising administration of the engineering agent. In some embodiments, the engineering agent is administered systemically. In some embodiments, the engineering agent is administered by intravenous or subcutaneous infusion or injection. In some embodiments, the engineering agent is administered locally. In some embodiments, the engineering agent is administered by intraperitoneal or intralesional infusion injection.

Some embodiments of these methods of treatment comprise administration of an effective amount of a compound or a composition disclosed herein. Some instances relate to a therapeutically (or prophylactically) effective amount. Other instances relate to a pharmacologically effective amount, that is an amount or dose that produces an effect that correlates with or is reasonably predictive of therapeutic (or prophylactic) utility. As used herein, the term “therapeutically effective amount” is synonymous with “therapeutically effective dose” and means at least the minimum dose of a compound or composition disclosed herein necessary to achieve the desired therapeutic or prophylactic effect. Similarly, a pharmacologically effective dose means at least the minimum dose of a compound or composition disclosed herein necessary to achieve the desired pharmacologic effect. Some embodiments refer to an amount sufficient to prevent or disrupt a disease process, or to reduce the extent or duration of pathology. Some embodiments refer to a dose sufficient to reduce a symptom associated with the disease or condition being treated. An effective dosage or amount of a compound or a composition disclosed herein can readily be determined by the person of ordinary skill in the art considering all criteria (for example, the rate of excretion of the compound or composition used, the pharmacodynamics of the compound or composition used, the nature of the other compounds to be included in the composition, the particular route of administration, the particular characteristics, history and risk factors of the individual, such as, e.g., age, weight, general health and the like, the response of the individual to the treatment, or any combination thereof) and utilizing his best judgment on the individual's behalf. Exemplary dosages are also disclosed in the Examples herein below.

In some embodiments, the immune cell in vivo engineering agent comprises a nucleic acid encoding a reprogramming agent packaged in a nanoparticle, wherein the reprogramming agent is a chimeric antigen receptor (CAR), an T cell receptor (TCR), or an immune cell engager, such as a bispecific T cell engager (BiTE). The CAR, TCR, or immune cell engager will generally bind to an antigen found on a tumor, autoimmunity-mediating, or other pathogenic cell. In some embodiments, the CAR, TCR, or immune cell engager binds to CD19, CD20, BCMA, mesothelin, PSMA, PSCA, or FAP. In some aspects, the nucleic acid encoding the reprogramming agent is an mRNA. In some aspects, the nanoparticle in which the nucleic acid encoding a reprogramming agent is packaged is a tropic lipid nanoparticle. In some aspects, the nanoparticle in which the nucleic acid encoding a reprogramming agent is packaged is targeted nanoparticle (tNP). In some aspects, the tNP is a targeted lipid nanoparticle (tLNP). In some aspects, the targeted nanoparticle comprises a binding moiety on its surface. In some aspects, the binding moiety comprises an antibody antigen binding domain. In some aspects, the binding moiety binds to a T cell or NK cell surface antigen. In some aspects, the nanoparticle is a tLNP comprising a binding moiety on its surface, wherein the binding moiety binds to a tumor surface antigen. In some aspects, the binding moiety comprises means for binding an immune cell. In some aspects, the binding moiety comprises means for binding CD2, CD5, or CD8.

Some of these disclosed methods of treatment relate to treatment of a particular disease or disorder in a subject in need thereof. In some embodiments, the subject is a human.

In some embodiments, the disease or disorder is an autoimmune disease or disorder for example, a T cell-mediated autoimmunity or a B cell-mediated (antibody-mediated) autoimmunity. In some instances, the B cell-mediated (antibody-mediated) autoimmune disease is systemic lupus erythematosus, neuromyelitis optica spectrum disorders, myasthenia gravis, pemphigus vulgaris, systemic sclerosis, antisynthetase syndrome (idiopathic inflammatory myopathy), multiple sclerosis, lupus nephritis, Sjörgen's syndrome, IgA nephropathy, myositis, or membranous nephropathy, severe combined immunodeficiency, or Fanconi anemia.

In some embodiments, the disease or disorder is a cancer. In some embodiments, the cancer is a hematologic cancer, for example, a lymphoma, leukemia, or myeloma. In some instances, the hematologic cancer is a B lineage or T lineage cancer. In some instances, the B lineage cancer is multiple myeloma, diffuse large B cell lymphoma, acute myeloid leukemia, Mantle Cell lymphoma, follicular lymphoma, B acute lymphoblastic leukemia and chronic lymphocytic leukemia, or myelodysplastic syndrome. In some embodiments, the cancer is a sarcoma. In some embodiments, the cancer is a carcinoma.

In some embodiments, the disease or disorder is a genetic disease or disorder. In some instances, the genetic disease or disorder is a hemoglobinopathy, for example, sickle cell disease or R-thalassemia.

In some embodiments, the disease or disorder is a fibrotic disease or disorder. In some instances, the fibrotic disease is cardiac fibrosis, arthritis, idiopathic pulmonary fibrosis, and Nonalcoholic steatohepatitis. In other instances, the disorder is involves tumor-associated fibroblasts.

### EXAMPLES

### Example 1: Conditioning Through Systemic Administration of a γ-Chain Receptor Cytokine (IL-15) for Priming of the Immune System Prior to In Vivo Reprogramming of the Immune System

Prior to administration of an in vivo immune cell engineering agent, patients are administered a continuous intravenous (CIV) infusion of recombinant human IL-15 at a rate of 1.0 to 2.0 μg/kg/day for at least 24 to 48 hours and up to 72 hours. The CIV is terminated on the day the engineering agent is first administered.

The immune cell engineering agent may be administered multiple times with IL-15 administered prior to only the initial administration of immune cell engineering agent, prior to each administration, or prior to any administration occurring more than 2 weeks after termination of the most recent administration of IL-15.

As a result of the IL-15 treatment there is an expansion of immune cells including T cells and NK cells, which are also activated to polyfunctional cells. Consequently, upon administration of an in vivo immune cell engineering agent there are more cells to transform, the proportion of cells transformed is increased, and the efficacy of transformed cells is increased due to a greater proportion of the transformed cells being polyfunctional.

### Example 2: Conditioning Through Targeted Administration a γ-Chain Receptor Cytokine (IL-15) for Priming of the Immune System Prior to, or in Conjunction with In Vivo Reprogramming of the Immune System

Prior to administration of an in vivo immune cell engineering agent, patients are administered a tLNP containing IL-15 encoding mRNA and targeted to the cancer to be treated. The amino acid sequence of the human IL-15 precursor (including signal sequence, residues 1-21) is:

(SEQ ID NO: 15; GenBank accession AAB97518)
MVLGTIDLCSCFSAGLPKTEANWVNVISDLKKIEDLIQSMHIDATLY
TESDVHPSCKVTAMKCFLLELQVISLESGDASIHDTVENLIILANNS
LSSNGNVTESGCKECEELEKKNIKEFLQSFVHIVQMFINTS

It is within the ability of one of skill in the art to convert the amino acid sequences into mRNA sequences encoding the cytokine. The IL-15 tLNP are administered by intravenous (IV) infusion or injection (or alternatively by intraperitoneal or intralesional infusion injection). The IL-15 tLNP are administered 3-4 days and 12-24 hours prior to the in vivo immune cell engineering agent. The IL-15 tLNP comprises a scFv on its exterior surface which binds a surface antigen expressed by the subject's tumor.

The immune cell engineering agent may be administered multiple times with the tLNP comprising the encoded IL-15 administered prior to only the initial administration of immune cell engineering agent, prior to each administration, or prior to any administration occurring more than 2 weeks after termination of the most recent administration of IL-15.

As a result of the IL-15 treatment there is an expansion of immune cells including T cells and NK cells, which are also activated to polyfunctional cells. Upon administration of an in vivo immune cell engineering agent there are more cells to transform, the proportion of cells transformed is increased, and the efficacy of transformed cells is increased due to a greater proportion of the transformed cells being polyfunctional.

### Example 3: Conditioning Through Systemic Administration of CTLA4 Blocking Agent to Enable In Vivo Reprogramming of the Immune System

The anti-CTLA-4 monoclonal antibody iplimumab is administered to a subject with cancer by intravenous infusion at a dose of 3 mg/kg, 2 to 14 days prior to, concurrent with, or up to 2 days after an administration of an in vivo T cell engineering agent. The anti-CTLA-4 monoclonal antibody can also be administered in the weeks following a course of multiple administrations of the immune cell engineering agent to promote development/activity of an endogenous anti-tumor immune response, for example, involving epitope spreading.

As a result of the anti-CTLA-4 treatment the number of functional Treg cells is reduced, engineered and endogenous anti-tumor T cells become activated/functional, and epitope spreading is promoted.

### Example 4: Conditioning Through Targeted Administration of CTLA4 Blocking Agent in Conjunction with In Vivo Reprogramming of the Immune System

An anti-CTLA-4 monoclonal antibody, (such as) iplimumab, is administered to a subject with cancer as a tLNP containing an encoding mRNA and targeted to the cancer to be treated. The amino acid sequences of the mature heavy and light chains of ipilimumab (as reported in US Pat. Pub. 20150283234) are:

>Ipilimumab heavy chain
(SEQ ID NO: 16)
QVQLVESGGGVVQPGRSLRLSCAASGFTFSSYTMHWVRQAPGK
GLEWVTFISYDGNNKYYADSVKGRFTISRDNSKNTLYLQMNSLRA
EDTAIYYCARTGWLGPFDYWGQGTLVTVSSASTKGPSVFPLAPSS
KSTSGGTAALGCLVKDYFPEPVTVSWNSGALTSGVHTFPAVLQSS
GLYSLSSVVTVPSSSLGTQTYICNVNHKPSNTKVDKRVEPKSCDK
THTCPPCPAPELLGGPSVFLFPPKPKDTLMISRTPEVTCVVVDVSH
EDPEVKFNWYVDGVEVHNAKTKPREEQYNSTYRVVSVLTVLHQD
WLNGKEYKCKVSNKALPAPIEKTISKAKGQPREPQVYTLPPSRDE
LTKNQVSLTCLVKGFYPSDIAVEWESNGQPENNYKTTPPVLDSDG
SFFLYSKLTVDKSRWQQGNVFSCSVMHEALHNHYTQKSLSLSPG
K
>Ipilimumab light chain
(SEQ ID NO: 17)
EIVLTQSPGTLSLSPGERATLSCRASQSVGSSYLAWYQQKPGQAP
RLLIYGAFSRATGIPDRFSGSGSGTDFTLTISRLEPEDFAVYYCQQ
YGSSPWTFGQGTKVEIKRTVAAPSVFIFPPSDEQLKSGTASVVCLL
NNFYPREAKVQWKVDNALQSGNSQESVTEQDSKDSTYSLSSTLT
LSKADYEKHKVYACEVTHQGLSSPVTKSFNRGEC

It is within the ability of one of skill in the art to convert the amino acid sequences into mRNA sequences encoding the antibody with signal sequences appropriate for the expression of an immunoglobulin. The anti-CTLA-4 mAb tLNP are administered by intravenous (IV) infusion or injection (or alternatively by intraperitoneal or intralesional infusion or injection). The anti-CTLA-4 mAb tLNP are administered 3-4 days and 12-24 hours prior to the in vivo immune cell engineering agent. Further administrations of the anti-CTLA-4 mAb tLNP are given periodically throughout the interval in which additional doses of the in vivo immune cell engineering agent are administered. The anti-CTLA-4 mAb tLNP comprises a scFv on its exterior surface which binds a surface antigen expressed by the subject's tumor.

As a result of the anti-CTLA-4 treatment the number of functional Treg cells is reduced, engineered and endogenous anti-tumor T cells become activated/functional, and epitope spreading is promoted.

### Example 5: Conditioning Through Targeted Administration of Chemokines to Facilitate In Vivo Reprogramming of the Immune System

Prior to administration of an in vivo immune cell engineering agent, patients are administered a tLNP containing CCL5 encoding mRNA and targeted to the cancer to be treated. The amino acid sequence of the human CCL5 precursor (including signal sequence, residues 1-23) is:

(SEQ ID NO: 18; UniProt accession P13501)
MKVSAAALAVILIATALCAPASASPYSSDTTPCCFAYIARPLPRAHIK
EYFYTSGKCSNPAVVFVTRKNRQVCANPEKKWVREYINSLEMS

It is within the ability of one of skill in the art to convert the amino acid sequences into mRNA sequence encoding the cytokine. The CCL5 tLNP are administered by intravenous (IV) infusion or injection (or alternatively by intraperitoneal or intralesional infusion or injection). The CCL5 tLNP are administered 3-4 days and 12-24 hours prior to the in vivo immune cell engineering agent. The CCL5 tLNP comprises a scFv on its exterior surface which binds a surface antigen expressed by the subject's tumor.

The in vivo immune cell engineering agent may be administered multiple times with the tLNP comprising the encoded CCL5 administered prior to only the initial administration of immune cell engineering agent, prior to each administration, or prior to any administration occurring more than 4 to 10 days after the most recent administration of CCL5. Further administrations of the tLNP comprising the encoded CCL5 are interposed between every 1, 2, or 3 administrations of the in vivo engineering agent being administered every 3 to 4 days.

The infusion of tumor targeted tLNP comprising CCL5 mRNA results in local recruitment and expansion of T cells, as well as other immune cells. Thus, the total number and percentage of immune effector cells locally within the tissue of interest amenable to in vivo reprogramming are increased. In addition to providing an activating conditioning effect, concurrent usage can also provide an adjuvant conditioning effect due to its generally ability to mobilize immune cells.

### Example 6: Conditioning Through Targeted Administration of a Biologically Active Agent, Flt3 Ligand, that Enhances the Activity of Antigen Presenting Cells, Thereby Augmenting In Vivo Reprogramming of the Immune System

Tumor cell-targeted tLNP in which Flt3 ligand-encoding mRNA is packaged are administered by intravenous infusion to a subject having cancer before, concurrently with, and/or subsequent to administration of an in vivo immune cell engineering agent. FLT3 ligand exists in soluble and integral membrane forms. The sequence of the soluble form (including signal sequence, residues 1-26) is:

(SEQ ID NO: 19; UniProt accession P49771-2)
MTVLAPAWSPTTYLLLLLLLSSGLSGTQDCSFQHSPISSDFAVKIR
ELSDYLLQDYPVTVASNLQDEELCGGLWRLVLAQRWMERLKTVA
GSKMQGLLERVNTEIHFVTKCAFQPPPSCLRFVQTNISRLLQETSE
QLVALKPWITRQNFSRCLELQCQPVETVFHRVSQDGLDLLTS

It is within the ability of one of skill in the art to convert the amino acid sequences into mRNA sequence encoding the cytokine. The Flt3 ligand tLNP are administered by intravenous (IV) infusion or injection (or alternatively by intraperitoneal or intralesional infusion or injection). When administered prior to the in vivo immune cell engineering agent, the Flt3 ligand tLNP are administered 3-4 days and 12-24 hours prior to the in vivo immune cell engineering agent. When administered concurrently with the in vivo immune cell engineering agent the Flt3 ligand tLNP is administered anytime the same day or 12-24 hours in advance for each of multiple administrations of the in vivo immune cell engineering agent. When administered subsequent to the in vivo immune cell engineering agent the Flt3 ligand tLNP is administered every 3-7 days while the tumor is shrinking, thereby promoting epitope spreading. The Flt3 ligand tLNP comprises a scFv on its exterior surface which binds a surface antigen expressed by the subject's tumor.

The infusion of tumor targeted tLNP comprising Flt3 ligand stimulate the activity of APCs and can lead to enhanced uptake, processing, presentation of tumor antigens and broadening of responding T cell repertoire against the tumors. Within solid tumors this also results in an expansion of the number and percentage of local immune effector cells that are amenable to reprogramming by the in vivo immune cell engineering agent, whether it delivers a CAR, TCR, or BiTE.

### Example 7: Conditioning Through Targeted Administration of Highly Active, Pan-Activating, Biological Response Modifiers in Conjunction with In Vivo Reprogramming of the Immune System

Tumor cell-targeted tLNP in which IL-12-encoding mRNA is packaged are administered by intravenous infusion to a subject having cancer before and/or subsequent to administration of an in vivo immune cell engineering agent. The tLNP delivering IL-12 is administered once or multiple times, every 3 to 4 days, with the last administration 1 day before the engineering agent. Optionally, administration is continued every 3 to 4 days concurrently with multiple administrations of the in vivo immune cell engineering agent or within 4 days or having been administered the in vivo immune cell engineering agent.

Active IL-12 is a heterodimeric cytokine composed of IL-12A and IL-12B chains encoded in separate genes. For efficient expression mRNA encoding both chains should be packaged in the same tLNP, either as two separate mRNAs or as a bicistronic mRNA. The sequence of IL-12A (including signal sequence, residues 1-22) is:

(SEQ ID NO: 20; UniProt accession P29459)
MCPARSLLLVATLVLLDHLSLARNLPVATPDPGMFPCLHHSQNLL
RAVSNMLQKARQTLEFYPCTSEEIDHEDITKDKTSTVEACLPLELT
KNESCLNSRETSFITNGSCLASRKTSFMMALCLSSIYEDLKMYQVE
FKTMNAKLLMDPKRQIFLDQNMLAVIDELMQALNFNSETVPQKSS
LEEPDFYKTKIKLCILLHAFRIRAVTIDRVMSYLNAS

The sequence of IL-12B (including signal sequence, residues 1-22) is:

(SEQ ID NO: 21; UniProt accession P29460)
MCHQQLVISWFSLVFLASPLVAIWELKKDVYVVELDWYPDAPGEM
VVLTCDTPEEDGITWTLDQSSEVLGSGKTLTIQVKEFGDAGQYTC
HKGGEVLSHSLLLLHKKEDGIWSTDILKDQKEPKNKTFLRCEAKNY
SGRFTCWWLTTISTDLTFSVKSSRGSSDPQGVTCGAATLSAERVR
GDNKEYEYSVECQEDSACPAAEESLPIEVMVDAVHKLKYENYTSS
FFIRDIIKPDPPKNLQLKPLKNSRQVEVSWEYPDTWSTPHSYFSLT
FCVQVQGKSKREKKDRVFTDKTSATVICRKNASISVRAQDRYYSS
SWSEWASVPCS

It is within the ability of one of skill in the art to convert the amino acid sequences into mRNA sequence encoding the cytokine. The Flt3 ligand tLNP are administered by intravenous (IV) infusion or injection (or alternatively by intraperitoneal or intralesional infusion or injection).

Infusion of tumor-targeted tLNP delivering IL-12 results in massive local activation of most if not all arms of cellular immunity within the microenvironment. It can also co-opt endogenous immunity as well as enabling the activity of tLNPs delivering in vivo immune cell engineering agent. Especially in the context of solid tumors, this approach expands the total number, percentage and activity of local immune effector cells that are amenable to reprogramming by the in vivo immune cell engineering agent.

### Example 8: Conditioning Through Use of Low Dose Cyclophosphamide to Enable In Vivo Reprogramming of the Immune System

Cyclophosphamide is generally thought of as a cytotoxic agent and is often used for lymphodepletion. However, at lower doses it has an immunomodulating effect that can enhance CAR or TCR therapy as delivered by an in vivo immune cell engineering agent. Treg cells have apparently greater sensitivity to cyclophosphamide so that metronomic dosing (50 mg daily or 100 mg every other day) shifts that balance toward a productive immune response. However, if dosing is continued over an extended period of time the Treg develop resistance.

Subjects with cancer are administered metronomic cyclophosphamide over a period of one week. The cyclophosphamide is then ceased, and the subjects are administered an in vivo immune cell engineering agent. The in vivo immune cell engineering agent can be administered at 3-4 day intervals. After 2-4 administrations of the in vivo immune cell engineering agent a new cycle of metronomic cyclophosphamide followed by the in vivo immune cell engineering agent and such cycles repeated until the cancer is eliminated or the treatment no longer has an effect.

### Example 9: Conditioning Through Use of IL-7 to Increase Translation of mRNA Delivered by Lipid Nanoparticles

LNPs encapsulating mRNA encoding modified mCherry were conjugated with an anti-CD5 antibody so they are targeted to CD5⁺ cells. CD5 is a marker expressed highly on the surface of T cells in both mice and humans. CD5 is also expressed at marginal/low levels on B cells/NK cells/myeloid cells.

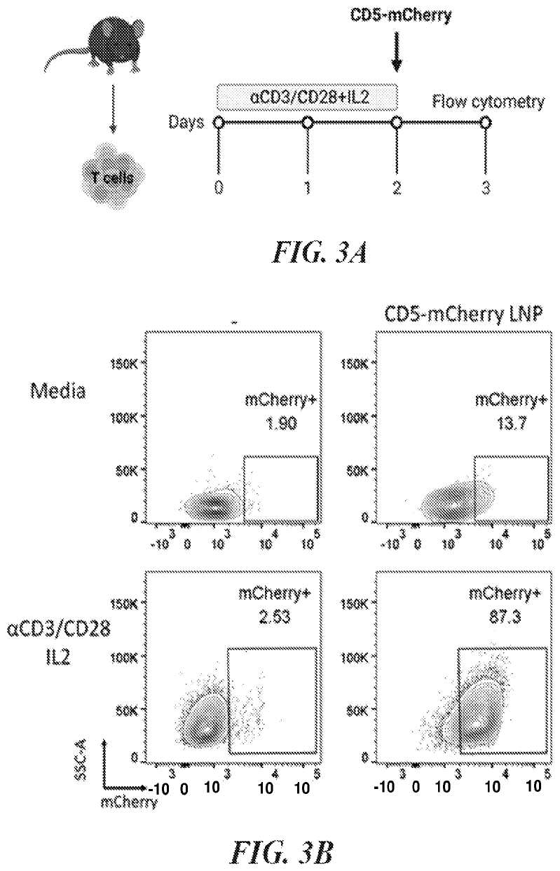

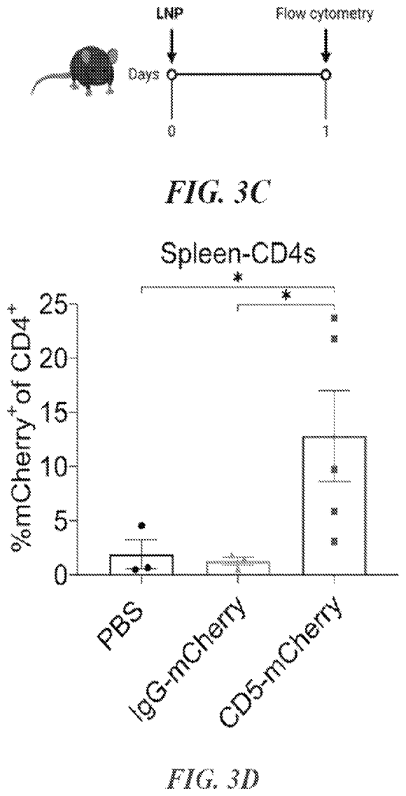

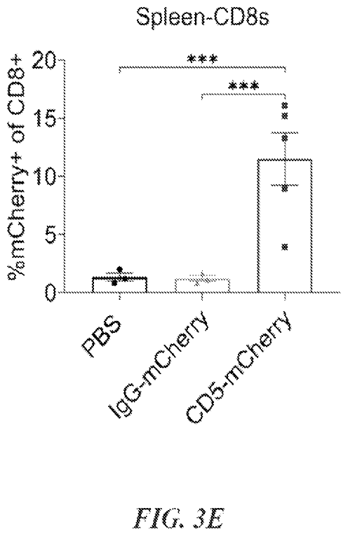

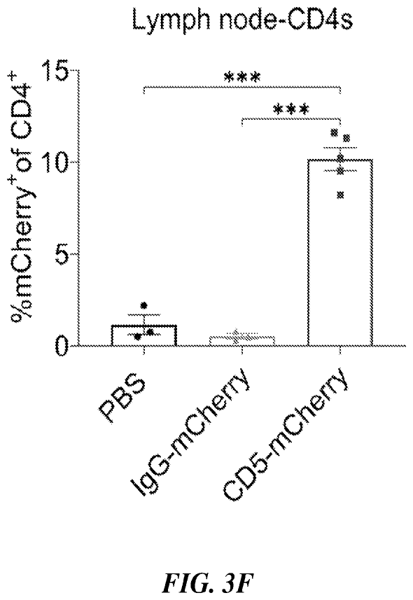

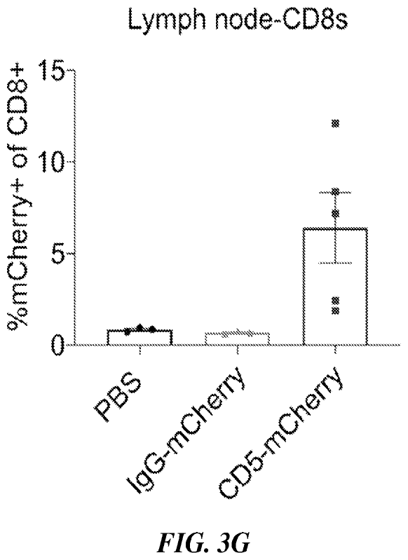

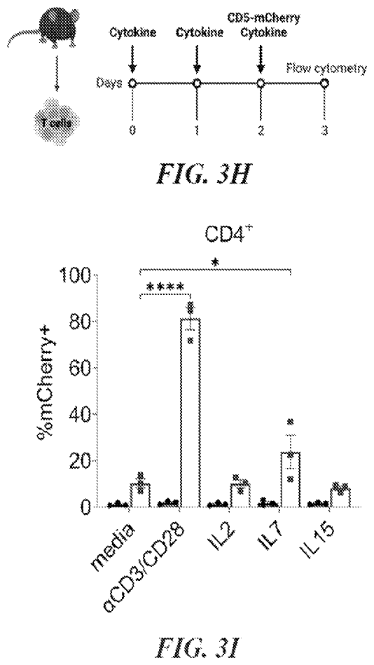

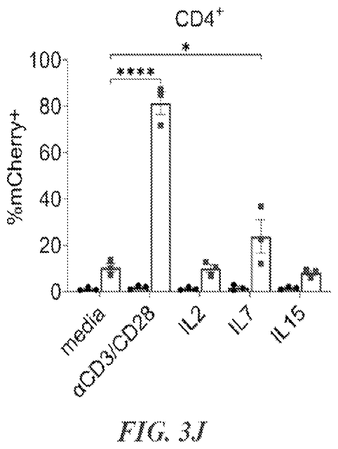

Using CD5-targeted tLNPs containing mCherry mRNA (referred to as CD5-mCherry tLNPs), mCherry expression was induced in vitro in T cells isolated from the spleens of C57/BL6 mice (FIG. 3A-3B). Low levels of expression were observed in ‘rested’ T cells (those cultured in T cell media alone). However, when T cells were activated by conditioning with αCD3/CD28 beads the transfection efficiency markedly increased from 13.7% of T cells expressing mCherry to 87.3% (FIG. 3B).

Next tLNPs were tested in vivo. Either IgG-mCherry or CD5-mCherry tLNPs were administered intravenously to mice and flow cytometry performed 24 hours later (FIG. 3C). Between 5 and 15% of CD4⁺ and CD8⁺ T cells were found in the spleen (FIG. 3D-E) and lymph node (FIG. 3F-G) were successfully transfected by CD5-mCherry targeted LNPs, as indicated by mCherry expression. LNPs conjugated with IgG induced no mCherry in T cells.

The effects of activating T cells using three common gamma chain cytokines-IL-2, IL-7 and IL-15 were also tested. IL-2 acts to stimulate the proliferation, activation, and effector function of T cells, along with promoting the survival and differentiation of memory T cells. IL-7 is critical for the development of T cells in the thymus, driving their maturation and differentiation. IL-7 also promotes the survival of T cells in the periphery, maintains T cell homeostasis and can enhance cytokine production by CD4⁺ and CD8 T cells⁺. IL-15 has similar effects, promoting the survival and proliferation of T cells, development of memory T cells, and enhances production of cytokines by T cells and direct cytotoxic activity of CD8⁺ T cells.

The amino acid sequence of the human IL-7 is:

(SEQ ID NO: 22; UniProt accession P13232)
MFHVSFRYIFGLPPLILVLLPVASSDCDIEGKDGKQYESVLMVSIDQ
LLDSMKEIGSNCLNNEFNFFKRHICDANKEGMFLFRAARKLRQFLK
MNSTGDFDLHLLKVSEGTTILLNCTGQVKGRKPAALGEAQPTKSL
EENKSLKEQKKLNDLCFLKRLLQEIKTCWNKILMGTKEH

The amino acid sequence of the human IL-2 is:

(SEQ ID NO: 23; UniProt accession P60568)
MYRMQLLSCIALSLALVTNSAPTSSSTKKTQLQLEHLLLDLQMILN
GINNYKNPKLTRMLTFKFYMPKKATELKHLQCLEEELKPLEEVLNL
AQSKNFHLRPRDLISNINVIVLELKGSETTFMCEYADETATIVEFLN
RWITFCQSIISTLT

The amino acid sequence of the human IL-15 is:

(SEQ ID NO: 24; UniProt accession P40933)
MRISKPHLRSISIQCYLCLLLNSHFLTEAGIHVFILGCFSAGLPKTEA
NWVNVISDLKKIEDLIQSMHIDATLYTESDVHPSCKVTAMKCFLLEL
QVISLESGDASIHDTVENLIILANNSLSSNGNVTESGCKECEELEEK
NIKEFLQSFVHIVQMFINTS

It is within the ability of one of skill in the art to convert the amino acid sequences into mRNA sequence encoding the cytokine.

First, T cells were isolated from the spleens of mice and cultured them in the presence of IL-2, IL-7, and IL-15 (FIG. 3H). Consistent with previous results, ‘rested’ T cells cultured in T cell media alone expressed low levels of mCherry after tLNP treatment while close to 80% of bead-activated T cells expressed mCherry within both the CD4⁺ and CD8⁺ subsets (FIG. 3I-3J). Although IL-2 and IL-15 had limited effects on increasing tLNP efficacy in vitro, IL-7 significantly increased mCherry expression in both CD4⁺ and CD8⁺ T cells, more than doubling the percent of cells expressing mCherry compared to resting T cells (FIG. 3I-3J).

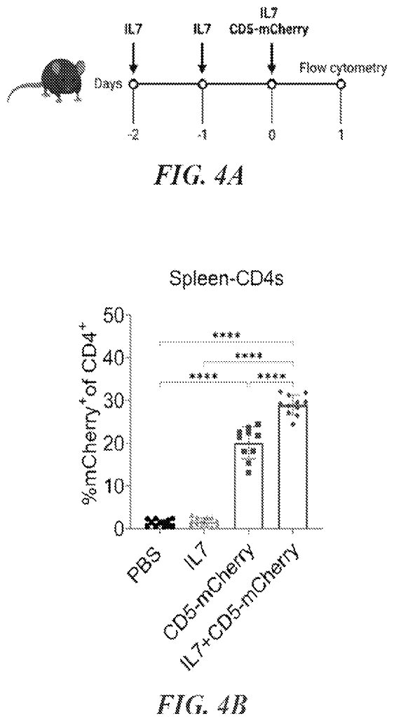

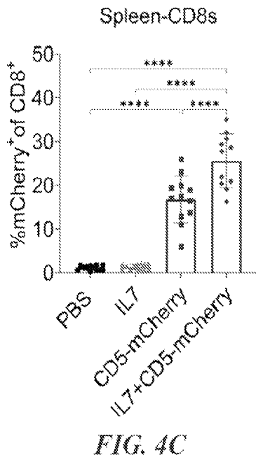

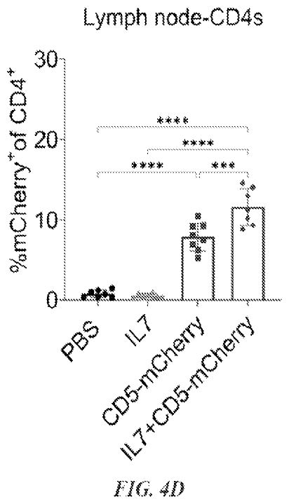

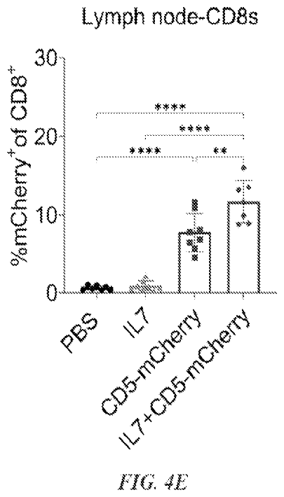

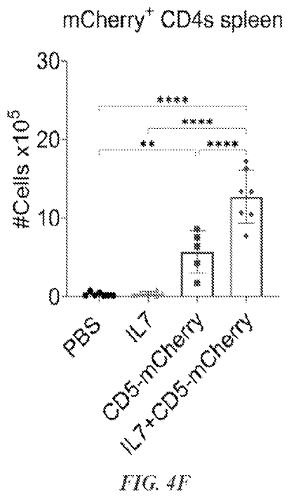

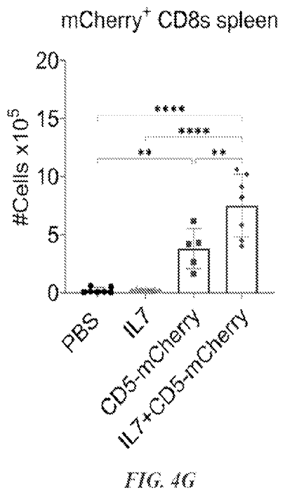

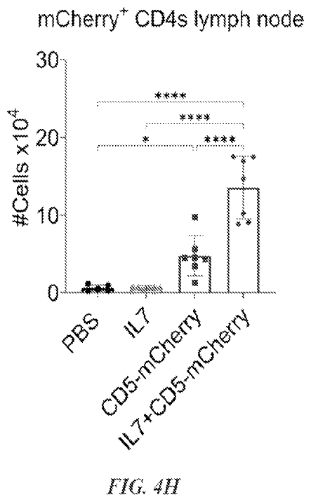

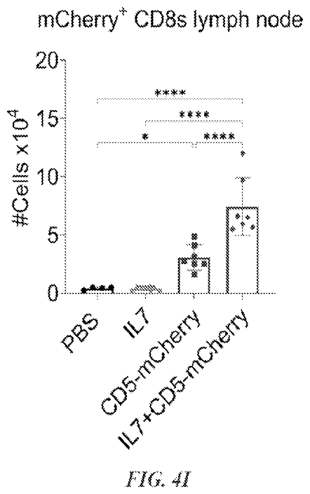

Given that IL-7 increased tLNP transfection in vitro, pre-treating mice with IL-7 was tested in vivo to determine if IL-7 had a similar effect in vivo. Mice were dosed with 5 μg IL-7 interperitoneally daily for three days. On the third day, mice also received CD5-mCherry tLNPs containing 10 μg of mCherry mRNA (FIG. 4A). The addition of IL-7 significantly increased the proportion of mCherry⁺ CD4⁺ and CD8⁺ T cells in the spleen and the lymph node (FIG. 4B-4E). Additionally, IL-7 also increased the total number of mCherry⁺ T cells (FIG. 4F-4I).

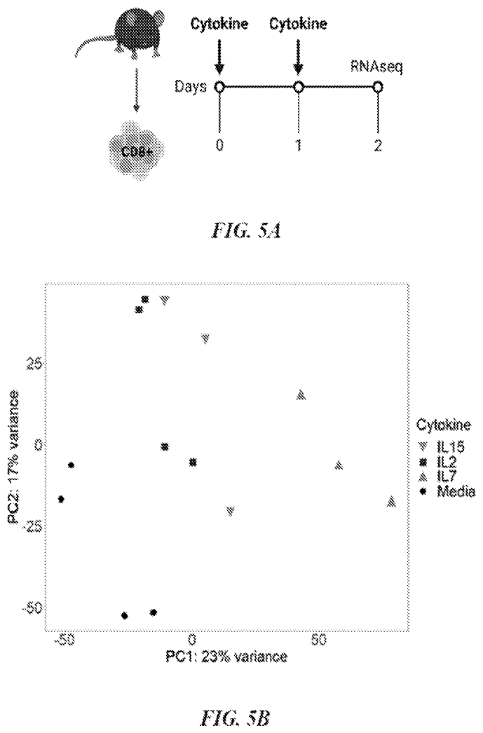

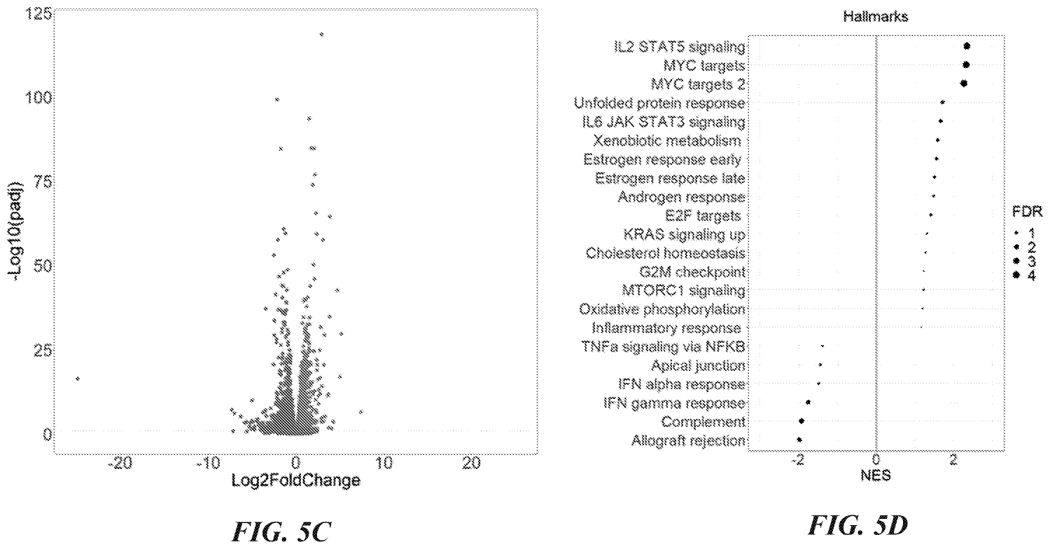

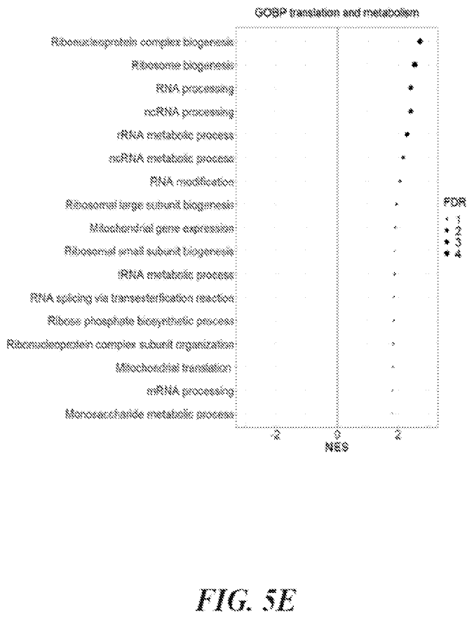

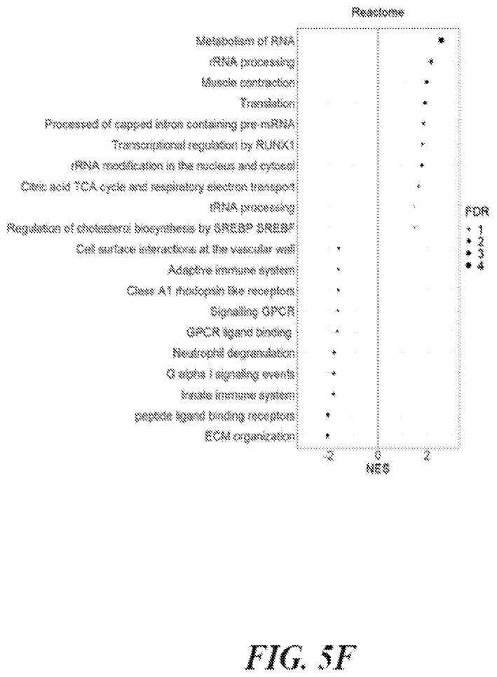

To identify how IL-7 increased the transfection rate of tLNPs, bulk RNA sequencing was performed on CD8⁺ T cells cultured in the presence of IL-2, IL-7 and IL-15. T cells were isolated from the spleens of C57BL/6 mice and cultured with T cell media alone or supplemented with IL-2, IL-7 or IL-15. 48 hours later, the cells were collected for sequencing (FIG. 5A). Principal component analysis showed a clear separation between IL-7 treated cells and cells treated with either IL-2 or IL-15 (FIG. 5B).

Differential gene analysis identified 1141 differentially expressed genes (581 up and 560 down) between IL-7 and IL-15 treated CD8⁺ T cells (FIG. 5C). Gene set enrichment analysis was performed using this list of differentially expressed genes against the hallmarks, reactome and gene ontology databases. IL-7 treatment was associated with the upregulation of IL-2 STAT5 signaling and MYC associated pathways, while inflammatory and type I and II IFN related genes were upregulated with IL-15 (FIG. 5D). Consistent with the results obtained from the hallmarks gene set collection, IL-15 treated CD8⁺ T cells upregulated reactome pathways associated with the immune system and interferons compared to IL-7 (FIG. 5E).

Genes associated with protein translation, RNA processing and cell metabolism were selectively enriched in IL-7 treated T cells (FIG. 5E-5F). Since a step in the expression of protein encoded by mRNA in tLNPs is the translation of the cargo mRNA into protein (whether that be a marker gene or CAR or some other agent), this observation indicates that a potential mechanism for the increased tLNP transfection efficiency observed after IL-7 treatment could be an increase of translation efficiency in the cells.

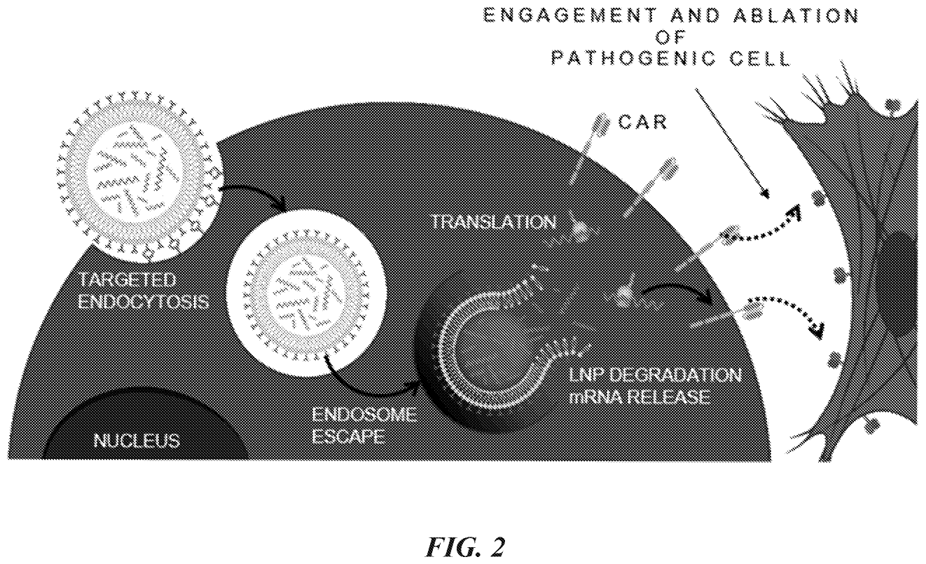

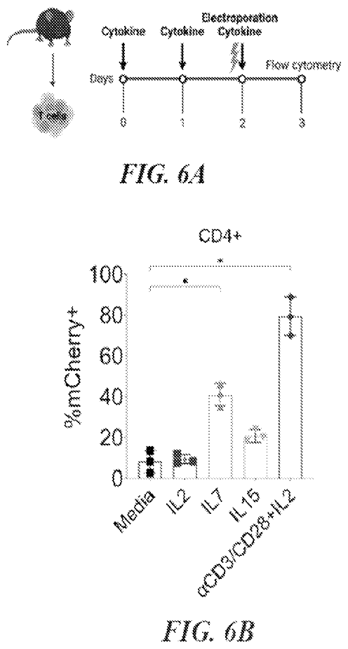

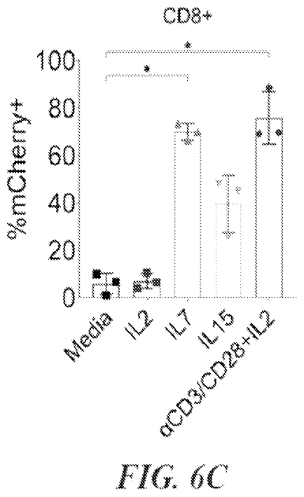

To directly assess the effects of IL-7 on protein translation, mCherry mRNA was electroporated into T cells cultured in T cell media alone or supplemented with IL-2, IL-7 or IL-15 (FIG. 6A). Since the transfection of T cells by tLNPs is a multistep process (FIG. 2), removal of the LNP component allows specific focus on translation.

Consistent with earlier studies using tLNPs, after electroporation, rested T cells expressed low levels of mCherry while up to 80% of αCD3/CD28 activated T cells were mCherry positive (FIG. 6B-6C). IL-2 treatment has little effect on increasing mCherry expression and IL-15 led to a small increase in expression in CD8⁺ T cells. IL-7 significantly increased mCherry expression after electroporation in both CD4⁺ and CD8⁺ T cells, with the effect more pronounced in the later.

These findings indicate that IL-7 increases protein translation in T cells, leading to increased expression of mRNA after electroporation.

### Example 10: Conditioning Through Use of IL-7 Pre-Treatment in Human T Cells Improves the Transfection Efficiency of CD5-mCherry tLNPs In Vitro

Human PBMCs were isolated from freshly acquired leukopaks from healthy donors and subsequently used to isolate T cells using a negative selection immunomagnetic cell separation method and cryopreserved until needed.

tLNP were prepared comprising ALC-0315:DSPC:cholesterol:DMG-PEG-2000:DSPE-PEG-2000-maleimide in a ratio of 50:10:38.5:1.4:0.1. The terminal group of the DMG-PEG2000 (non-functionalized PEG) was methoxy. The N/P ratio (the ratio of positively-chargeable lipid amine (N=nitrogen) groups to negatively-charged nucleic acid phosphate (P) groups) was 6. After initial LNP formation a SATA-modified anti-CD5 antibody was reacted with the maleimide moiety to provide the final tLNP. The payload was CleanCap® mCherry 5-methoxyuridine (5moU) mRNA encoding the fluorescent protein mCherry (Trilink).

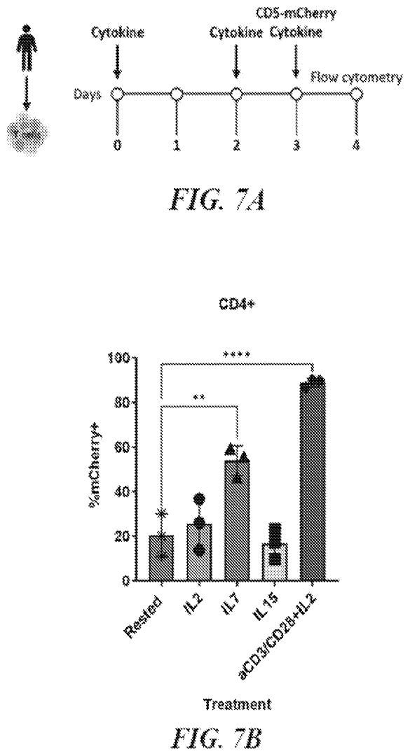

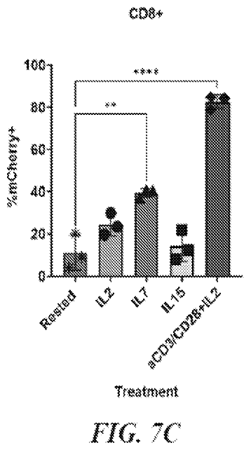

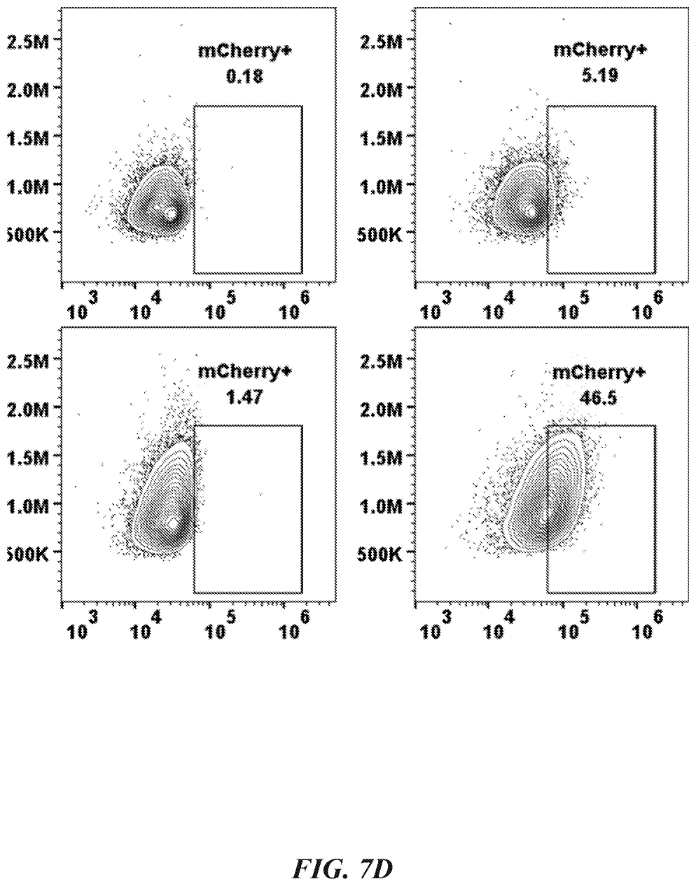

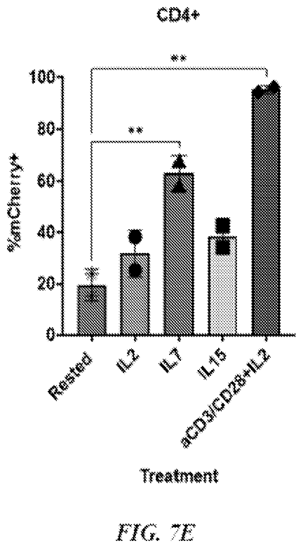

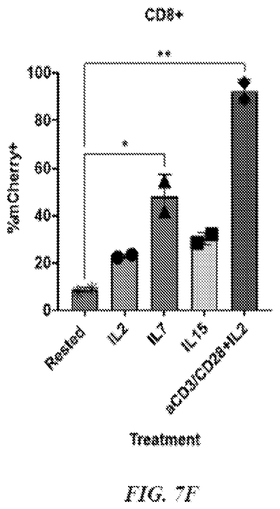

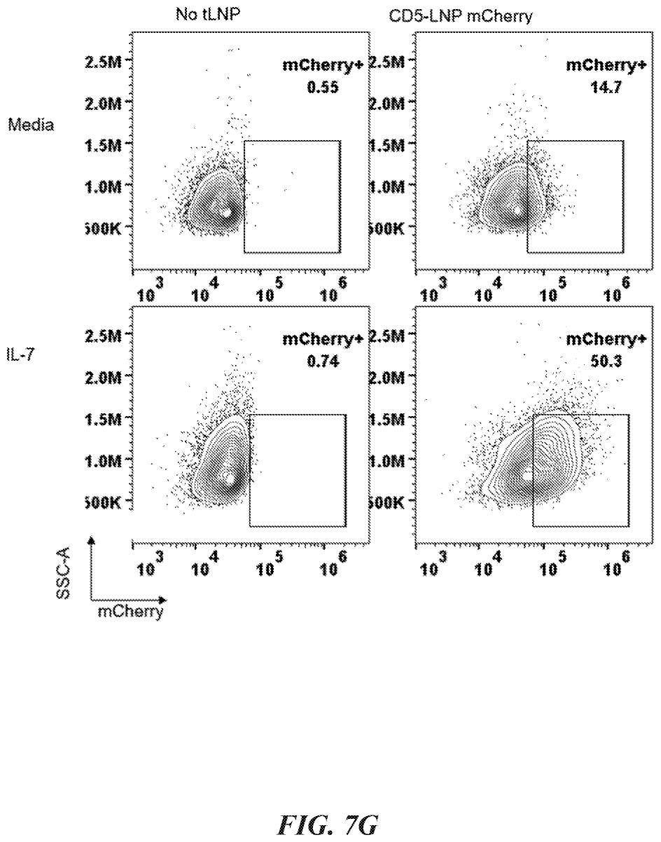

T cells were thawed and separately cultured with: a) rhIL-2 (11 ng/ml), b) rhIL-7 (15 ng/ml), c) rhIL-15 (20 ng/ml), or with d) anti-CD3/CD28 beads and rhIL-2 (11 ng/ml) to activate the T cells (see Table 4 for cytokine details). After 48 hours, the T cells were collected, counted, and resuspended in fresh media with fresh cytokines (FIG. 7A). Three experiments were conducted using cells from two different donors (two experiments from a first donor and one from a second donor).

TABLE 4
Concentration of Cytokines Used in Example 10

| Interleukins | Concentration ng/ml |
|---|---|
| Control (Basal Media only) | 0 |
| IL-2 | 11 |
| IL-7 | 15 |
| IL-15 | 20 |

At 72 hours, the cells were collected, counted, and resuspended in fresh media with fresh cytokines, and plated in U-bottom 96 well plates in triplicate at 2×10⁵ cells per well. The activated T cells were debeaded prior to counting and resuspended in media containing rhIL-2 (11 ng/ml). The cells were transfected with 600 ng CD5-tLNP-mCherry (MB22_00044_C2, see Table 1 for tLNP composition) for 1 hour and washed with PBS twice to remove excess tLNP and minimize non-specific uptake. The transfected cells were then incubated as before for a further 23 hours, for a total of 24 hours post transfection.

At 24 hours post transfection, the cells were washed and stained with a viability dye and labelled with anti-CD3 (pan-T cell marker), anti-CD4, and anti-CD8 antibodies to distinguish between the different T cell subpopulations, and an anti-CD25 antibody (a T cell activation marker). See Table 5 for flow cytometry antibody details. The labelled cells were then analyzed on a flow cytometer, and the flow cytometry data is shown in FIG. 7D. The data was then analyzed using FlowJo version 10.8.1 and a gating strategy was established to exclude dead cells using the viability dye, followed by gating on CD3+ cells (pan-T cell), and further gating on the two T cell subpopulations CD4+ and CD8+ cells. Last, the expression of mCherry (average of the triplicate samples) was recorded on the gated CD4+ cells (FIG. 7B) and CD8+ cells (FIG. 7C) for each cell culture condition.

TABLE 5
Flow Cytometry Antibodies

| Antibody | Color |
|---|---|
| Live Dead (Zombie Aqua) | AmCyan |
| CD3 | BV605 |
| CD4 | PE-Cy7 |
| CD8 | AF700 |
| tLNP-mCherry | PE/Texas Red |
| CD25 | FITC |

For both CD4+ and CD8+ T cells, IL-7 pretreatment of the T cells provided a significant increase in the percentage of cells expressing mCherry (FIG. 7B-7C) and in the level of expression (FIG. 7D).

This experiment was repeated using a tLNP comprising a different ionizable cationic lipid, CICL1 instead of ALC-0315. The tLNP had the composition CICL1:DSPC:CHOL:DMG-PEG2000:DSPE-PEG2000-MAL [58:10:30.5:1.4:0.1] and encapsulated mCherry mRNA with N/P=6. In this case the U bases in the mCherry mRNA were substituted with N¹-methylpseudouridine and the anti-CD5 antibody was a variant of the one previously used. The protocol was otherwise essentially as described above with cells from two different donors cultured without or with cytokine or beads and transfected in triplicate.

Again, for both CD4+ and CD8+ T cells, IL-7 pretreatment of the T cells provided a significant increase in the percentage of cells expressing mCherry (FIG. 7E-7F) and in the level of expression (FIG. 7G). The percentage of cells expressing mCherry was higher for all three of the cytokines (IL-2, IL-7, and IL-15) upon transfection with the CICL1-containing tLNPs that had been observed with the ALC-0315-containing tLNPs.

### Example 11: Ex Vivo Conditioning of and Transfection of T Cells

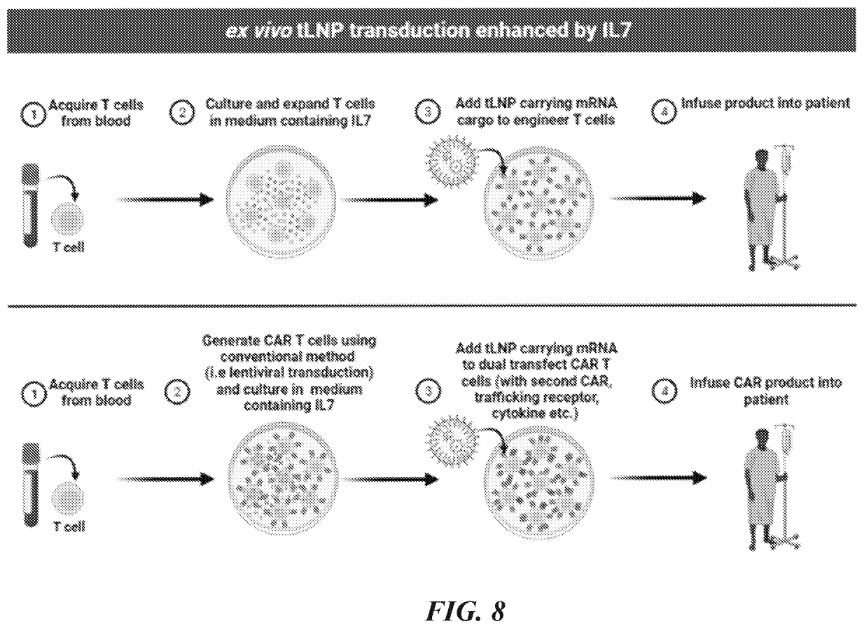

As an alternative to in vivo use of tLNP, conditioning and transfection can take place ex vivo, similar to the Example 10, but in a clinical setting. Cells are acquired from a patient, for example by apheresis. T cells are cultured and expanded in media containing IL-7. The T cells are contacted with a tLNP comprising a therapeutic payload (for example, a CAR) and reinfused into the patient (FIG. 8).

Further alternatives include conventional viral transduction which could be followed by tLNP-mediated transfection of a second therapeutic agent prior to reinfusion into the patient (FIG. 8).

### Example 12: Transfection of Tumor Cells with tLNP

tLNP were prepared comprising CICL1:DSPC:CHOL:DMG-PEG2000:DSPE-PEG2000-MAL [58:10:30.5:1.4:0.1], encapsulating mCherry mRNA with N/P=6. After initial LNP formation a SATA-modified antibody was reacted with the maleimide moiety to provide the final tLNP. Each of the following antibodies were individually conjugated to LNPs: 47G4 and FMC63 for targeting CD19, Leu16 and 2.1.2 for targeting CD20, a chimeric RPA-T8 for targeting CD8, a humanized 5D7 for targeting CD5, and as non-targeting controls cetuximab (anti-EGFR) and teropavinab (anti-HIV gp120). The Fc portion of each of the antibodies had also been modified to disrupt Fc receptor binding (L234A/L235A/P329A).

The antibody-conjugated LNP were used to transfect: the human B cell tumor lines Raji (CD19+CD20+), NALM6 (CD19+CD20low or dim), and Daudi (CD19+CD20+), RPM18226 (CD19−CD20low or dim), and JeKo (CD19+CD20+): the human T cell tumor lines Jurkat (CD5+CD8−) and HPB-ALL (CD5+CD8+); healthy unactivated pan-B cells isolated from PBML; and expanded T cells from two donors (D1 and D2). One hour after adding antibody-conjugated LNP containing 0.6 μg of mRNA to the cultures in duplicate, the cells were washed and 24 hours after transfection they were analyzed by flow cytometry to detect and quantitate mCherry expression.

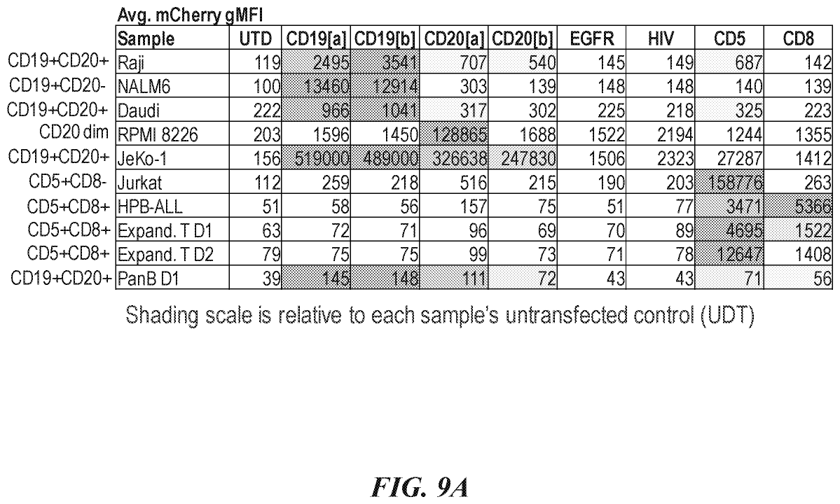

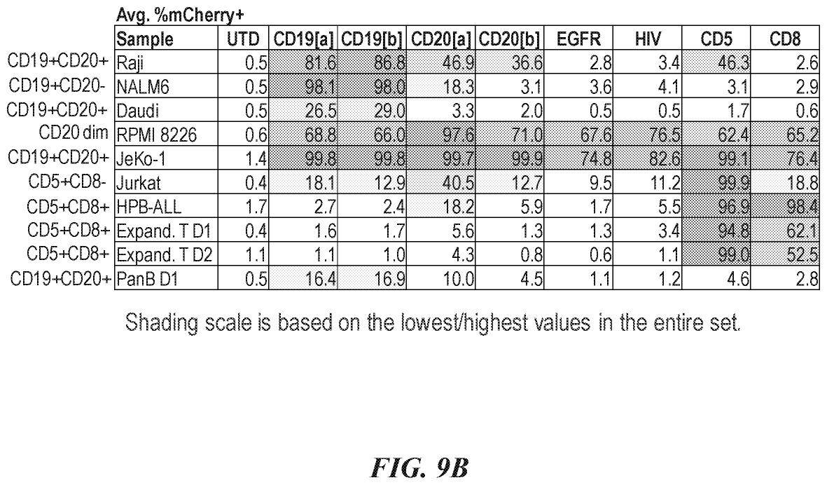

As seen in FIGS. 9A-B, anti-CD19 or anti-CD20 antibody-conjugated LNPs effectively and selectively delivered the mCherry payload to specific human B tumor cell lines, including Raji (CD19+CD20+), NALM6 (CD19+CD20low or dim), and Daudi (CD19+CD20+). However, transfection of non-activated primary pan B cells, though target specific, was relatively poor. Unexpectedly, RPMI 8226 (CD19−CD20low or dim) and JEKO-1 (CD19+CD20+) displayed non-selective uptake of tLNPs in an antigen-independent manner, as non-targeting LNP could comparably deliver the mCherry payload to these cells, although it is known that some tumor cell lines have this property.

Anti-CD5 and anti-CD8 antibody-conjugated LNPs also exhibited successful delivery and expression of the mCherry payload to human T cell leukemia cell lines in vitro, including Jurkat (CD5+CD8−) and HPB-ALL (CD5+CD8+). Furthermore, the same tLNPs successfully engineered primary expanded human T cells.

The ability of CD19 to mediate tLNP uptake in B cell tumor lines to an extent comparable to CD5 (a scavenger receptor) in T cell tumor lines was a major surprise and bodes well for the ability of CD19-targeted LNP to deliver a variety of payloads to tumor cells in vivo to condition the tumor environment to be more susceptible to immunologic attack.

### Enumerated Embodiments

The following listing of enumerated embodiments exemplifies how the disclosed aspects may be expressed in varying scope but is in no way exhaustive.

1. A method of conditioning a subject who receives an engineering agent comprising providing a conditioning agent to the subject by systemic administration prior to, concurrently with, or subsequent to administration of the engineering agent, wherein the conditioning agent comprises an immune checkpoint inhibitor.

2. The method of embodiment 1 wherein the systemic administration of the immune checkpoint inhibitor is by intravenous or subcutaneous infusion or injection.

3. The method of embodiment 1 or 2, wherein the systemic administration of the immune checkpoint inhibitor occurs at 3-week intervals.

4. The method of embodiment 3, wherein a first administration of the engineering agent occurs about 1 week after a 2^{nd} systemic administration of the immune checkpoint inhibitor.

5. The method of any one of embodiments 1-4, wherein the immune checkpoint inhibitor comprises an anti-CTLA-4 antibody.

6. The method of any one of embodiments 1-5, wherein the immune checkpoint inhibitor comprises an anti-PD-1, anti-PD-L1, anti-Tim-3, or anti-LAG-3 antibody.

7. The method of any one of embodiments 1-6, wherein conditioning reduces Treg cell activity.

8. The method of any one of embodiments 1-7, wherein conditioning activates T effector cells.

9. The method of any one of embodiments 1-8, wherein conditioning mobilizes immune cells into a tumor or other locus of disease.

10. A method of conditioning a subject who receives an engineering agent comprising providing a conditioning agent, wherein the conditioning agent comprises an agent that enhances the activity of antigen presenting cells, to the subject prior to, concurrently with, or subsequent to administration of the engineering agent.

11. The method of embodiment 10, wherein the administration of the agent that enhances the activity of antigen presenting cells is by intravenous, intralesional, or intraperitoneal infusion or injection.

12. The method of any one of embodiments 10 or 11, wherein the agent that enhances the activity of antigen presenting cells is provided 3-4 days and 12-24 hours prior to the in vivo immune cell engineering agent.

13. The method of any one of embodiments 10-12, wherein the agent that enhances the activity of antigen presenting cells is provided anytime the same day as or 12-24 hours in advance of each of multiple administrations of the in vivo immune cell engineering agent.

14. The method of any one of embodiments 10-13, wherein the agent that enhances the activity of antigen presenting cells is provided every 3-7 days subsequent to a pause in or conclusion of treatment with the in vivo immune cell engineering agent while the tumor is shrinking.

15. The method of any one of embodiments 10-14, wherein the agent that enhances the activity of antigen presenting cells comprises Flt-3 ligand or gm-CSF.

16. The method of any one of embodiments 10-15, wherein epitope spreading is promoted.

17. The method of any one of embodiments 10-16, wherein polyfunctional effector cells are expanded.

18. A method of conditioning a subject who receives an engineering agent comprising administering low-dose cyclophosphamide prior to administration of the engineering agent.

19. The method of embodiment 18, wherein the cyclophosphamide is administered with metronomic dosing.

20. The method of embodiment 18 or 19 wherein the cyclophosphamide is administered at a dose of 50 mg daily or 100 mg every other day.

21. The method of any one of embodiments 18-20, wherein the cyclophosphamide is administered over a period of 5 to 8 days.

22. The method of any one of embodiments 18-21 wherein the cyclophosphamide is administered at a daily dose of 10-50 mg for up to 3 days.

23. The method of any one of embodiments 18-22, wherein the engineering agent is administered 3 to 4 days after a last dose of the cyclophosphamide.

24. The method of any one of embodiments 18-23, whereby Treg cell activity is reduced.

25. A method of adjuvant conditioning for a subject who receives an engineering agent comprising providing an activating conditioning agent prior to or concurrently with the engineering agent, wherein the activating conditioning agent comprises a γ-chain receptor cytokine, an inflammatory chemokine, a pan-activating cytokine, an antigen presenting cell activity enhancer, or a CTLA-4 checkpoint inhibitor.

26. The method of embodiment 25, whereby the number of polyfunctional immune effector cells is expanded or immune effector cells are mobilized.

27. A method of adjuvant conditioning for a subject who receives an engineering agent comprising providing an adjuvant conditioning agent concurrently with or after an in vivo engineering agent, wherein the adjuvant conditioning agent comprises an immune checkpoint inhibitor, low-dose cyclophosphamide, a γ-chain receptor cytokine, an antigen presenting cell activity enhancer, an anti-CCR4 antibody, or a pan-activating cytokine.

28. The method of embodiment 27, whereby Treg cell activity is reduced, or endogenous immunity is recruited to a tumor or other locus of disease.

29. A method of conditioning a subject who receives an engineering agent comprising providing a nanoparticle comprising a nucleic acid encoding a conditioning agent to the subject prior to administration of the engineering agent, wherein the conditioning agent comprises a γ-chain receptor cytokine or other γ-chain receptor agonist.

30. The method of embodiment 29, wherein the nanoparticle is administered by intravenous or subcutaneous infusion or injection.

31. The method of embodiment 29 or 30, wherein the nanoparticle is provided to the subject by 3 weekly administrations and the third administration is 3 to 7 days before the subject receives the engineering agent.

32. The method of any one of embodiments 29-31, wherein the γ-chain receptor cytokine comprises IL-15, IL-2, IL-7, or IL-21.

33. The method of any one of embodiments 29-32, wherein conditioning increases the number of polyfunctional immune effector cells.

34. The method of any one of embodiments 29-33, wherein conditioning leads to mobilization of reprogrammed cells into a tumor or other locus of disease subsequent to administration of the engineering agent.

35. A method of conditioning a subject who receives an engineering agent comprising providing a nanoparticle comprising a nucleic acid encoding a conditioning agent to the subject prior to, concurrently with, or subsequent to administration of the engineering agent, wherein the conditioning agent comprises an immune checkpoint inhibitor.

36. The method of embodiment 35, wherein the nanoparticle is administered by intravenous or subcutaneous infusion or injection.

37. The method of embodiment 35 or 36, wherein administration of the nanoparticle comprising the nucleic acid encoding the immune checkpoint inhibitor occurs every 3 to 7 days over a period of 1 week to 1 month.

38. The method of embodiment 37, wherein a first administration of the engineering agent occurs at least about 2 weeks after a first administration of the nanoparticle comprising the nucleic acid encoding the immune checkpoint inhibitor.

39. The method of any one of embodiments 35-38, wherein the immune checkpoint inhibitor is an anti-CTLA-4 antibody.

40. The method of any one of embodiments 35-39, wherein the immune checkpoint inhibitor is an anti-PD-1, anti-PD-L1, anti-Tim-3, or anti-LAG-3 antibody.

41. The method of any one of embodiments 35-40, wherein conditioning reduces Treg cell activity.

42. The method of any one of embodiments 35-41, wherein conditioning activates T effector cells.

43. The method of any one of embodiments 35-42, wherein conditioning mobilizes immune cells into a tumor or other locus of disease.

44. A method of conditioning a subject who receives an engineering agent comprising administering a conditioning agent, wherein the conditioning agent comprises a nanoparticle comprising a nucleic acid encoding an inflammatory chemokine.

45. The method of embodiment 44, wherein the administration of the nanoparticle comprising the nucleic acid encoding the inflammatory chemokine is by intravenous, intralesional, or intraperitoneal infusion or injection.

46. The method of embodiment 44 or 45, wherein the nanoparticle comprising the nucleic acid encoding the inflammatory chemokine is administered at 3- to 4-day intervals.

47. The method of embodiment 46, wherein the nanoparticle comprising the nucleic acid encoding the inflammatory chemokine is administered 2, 3, or 4 times prior to administration of the engineering agent.

48. The method of embodiment 46 or 47, wherein the engineering agent is administered the day following the most recent administration of the nanoparticle comprising the nucleic acid encoding the inflammatory chemokine.

49. The method of embodiment 48, wherein the nanoparticle comprising the nucleic acid encoding the inflammatory chemokine is administered following every 1, 2, or 3 administrations of the in vivo engineering agent.

50. The method of any one of embodiments 44-49, wherein the inflammatory chemokine comprises CCL2, CCL3, CCL4, CCL5, CCL11, CXCL1, CXCL2, CXCL-8, CXCL9, CXCL10, or CXCL11.

51. The method of any one of embodiments 44-50, wherein the inflammatory chemokine comprises CCL5.

52. The method of any one of embodiments 44-51, wherein the conditioning expands and/or mobilizes immune cells to a tumor or other locus of disease.

53. A method of conditioning a subject who receives an engineering agent comprising providing a nanoparticle comprising a nucleic acid encoding a conditioning agent to the subject prior to, concurrently with, or subsequent to administration of the engineering agent, wherein the conditioning agent comprises an agent that enhances activity of antigen presenting cells.

54. The method of embodiment 53, wherein the administration of a nanoparticle comprising a nucleic acid encoding the agent that enhances the activity of antigen presenting cells, is by intravenous, intralesional, or intraperitoneal infusion or injection.

55. The method of embodiment 53 or 54, wherein the agent that enhances the activity of antigen presenting cells is provided 3-4 days and 12-24 hours prior to the in vivo immune cell engineering agent.

56. The method of any one of embodiments 53-55, wherein the agent that enhances the activity of antigen presenting cells is provided anytime the same day as or 12-24 hours in advance of each of multiple administrations of the in vivo immune cell engineering agent.

57. The method of any one of embodiments 53-56, wherein the agent that enhances the activity of antigen presenting cells is provided every 3-7 days subsequent to a pause in or conclusion of treatment with the in vivo immune cell engineering agent while the tumor is shrinking.

58. The method of any one of embodiments 53-57, wherein the agent that enhances the activity of antigen presenting cells comprises Flt-3 ligand, gm-CSF, or IL-18.

59. The method of any one of embodiments 53-58, wherein epitope spreading is promoted.

60. A method of conditioning a subject who receives an engineering agent comprising administering a nanoparticle comprising a nucleic acid encoding a conditioning agent prior or subsequent to administration of the engineering agent, wherein the conditioning agent comprises a pan-activating cytokine.

61. The method of embodiment 60, wherein the administration of the nanoparticle comprising the nucleic acid encoding the pan-activating cytokine is by intravenous, intralesional, or intraperitoneal infusion or injection.

62. The method of embodiment 60 or 61, wherein the nanoparticle comprising the nucleic acid encoding the pan-activating cytokine is administered at 3- to 4-day intervals.

63. The method of embodiment 62, wherein the nanoparticle comprising the nucleic acid encoding the pan-activating cytokine is administered 1, 2, 3, or 4 times prior to administration of the engineering agent which is administered 1 to 7 days after the most recent administration of the nucleic acid encoding the pan-activating cytokine.

64. The method of embodiment 62 or 63, wherein the nanoparticle comprising the nucleic acid encoding the pan-activating cytokine is administered within 4 days following the most recent administration of the engineering agent.

65. The method of any one of embodiments 62-64, wherein the pan-activating cytokine comprises IL-12 or IL-18.

66. The method of any one of embodiments 62-65, wherein the conditioning activates immune cells in a tumor or other locus of disease.

67. The method of any one of embodiments 1-66, wherein polyfunctional effector cells are expanded.

68. The method of any one of embodiments 1-67, wherein the nanoparticle in which the conditioning agent is provided is a targeted nanoparticle.

69. The method of embodiment 68, wherein the targeted nanoparticle comprises a binding moiety on its surface.

70. The method of embodiment 69, wherein the binding moiety comprises an antibody antigen binding domain.

71. The method of any one of embodiments 69 or 70, wherein the binding moiety binds to a tumor surface antigen.

72. The method of any one of embodiments 69-71, wherein the nanoparticle is a lipid nanoparticle.

73. The method of any one of embodiments 1-72, wherein the nanoparticle in which the conditioning agent is provided is a tropic lipid nanoparticle.

74. The method of any one of embodiments 1-73, wherein the nucleic acid encoding the conditioning agent is an mRNA.

75. The method of any one of embodiments 1 to 74, wherein the engineering agent comprises a nucleic acid encoding a reprogramming agent packaged in a nanoparticle, wherein the reprogramming agent is a chimeric antigen receptor (CAR), an T cell receptor (TCR), or an immune cell engager.

76. The method of embodiment 75, wherein the immune cell engager is a bispecific T cell engager (BiTE).

77. The method of embodiment 75 or 76, wherein the nucleic acid encoding the reprogramming agent is an mRNA.

78. The method of any one of embodiments 75 to 77, wherein the nanoparticle in which the nucleic acid encoding a reprogramming agent is packaged is a tropic lipid nanoparticle.

79. The method of any one of embodiments 75 to 78, wherein the nanoparticle in which the nucleic acid encoding the reprogramming agent is packaged is targeted nanoparticle (tNP).

80. The method of embodiment 79, wherein the targeted nanoparticle comprises a binding moiety on its surface.

81. The method of embodiment 80, wherein the binding moiety comprises an antibody antigen binding domain.

82. The method of embodiment 79 or 80, wherein the binding moiety binds to a T cell or NK cell surface antigen.

83. The method of embodiment 82, wherein the binding moiety comprises means for binding an immune cell.

84. The method of embodiment 83, wherein the binding moiety binds to a tumor surface antigen.

85. The method of embodiment 80, wherein the binding moiety binds to CD5.

86. The method of embodiment 80, wherein the binding moiety binds to CD8.

87. The method of embodiment 80, wherein the binding moiety binds to CD2.

88. The method of any one of embodiments 79 to 87, wherein the tNP is a targeted lipid nanoparticle (tLNP).

89. A method of treatment comprising the method conditioning of any one of embodiments 1-88 further comprising administration of an engineering agent.

90. The method of treatment of embodiment 89, wherein the engineering agent comprises a nucleic acid encapsulated in a tLNP.

91. A use of a conditioning agent for conditioning a subject who receives an engineering agent, wherein conditioning the subject comprises providing a nanoparticle comprising a nucleic acid encoding the conditioning agent to the subject prior to, concurrently with, or subsequent to administration of the engineering agent, wherein the conditioning agent comprises a γ-chain receptor agonist, an inflammatory chemokine, a pan-activating cytokine, an antigen presenting cell activity enhancer, a CTLA-4 checkpoint inhibitor, an immune checkpoint inhibitor or an anti-CCR4 antibody.

92. The use of embodiment 91, wherein the conditioning agent comprises a γ-chain receptor agonist.

93. The use of embodiment 92, wherein the nanoparticle is administered by intravenous or subcutaneous infusion or injection.

94. The use of embodiment 92 or 93, wherein the nanoparticle is provided to the subject by 3 weekly administrations.

95. The use of embodiment 94, wherein the third administration is 3 to 7 days before the subject receives the engineering agent, whereby the conditioning is activating conditioning.

96. The use of any one of embodiments 91 to 95, wherein conditioning increases the number of polyfunctional immune effector cells.

97. The use of embodiment 94, wherein at least one of the weekly administrations occurs after the subject receives the engineering agent, whereby the conditioning is adjuvant conditioning.

98. The use of any one of embodiments 91-94, or 97, wherein conditioning leads to mobilization of reprogrammed cells into a tumor or other locus of disease subsequent to administration of the engineering agent.

99. The use of embodiment of any one of embodiments 91 to 98, wherein the γ-chain receptor agonists comprises a γ-chain receptor cytokine.

100. The use of embodiment 99, wherein the γ-chain receptor cytokine comprises IL-15, IL-2, IL-7, or IL-21.

101. The use of embodiment 91, wherein the conditioning agent comprises an immune checkpoint inhibitor.

102. The use of embodiment 101, comprising providing the nanoparticle comprising the nucleic acid encoding the immune checkpoint inhibitor to the subject prior to, concurrently with, or subsequent to administration of the engineering agent.

103. The use of embodiment 101 or 102, wherein the nanoparticle is administered by intravenous or subcutaneous infusion or injection.

104. The use of any one of embodiments 101 to 103, wherein administration of the nanoparticle comprising the nucleic acid encoding the immune checkpoint inhibitor occurs every 3 to 7 days over a period of 1 week to 1 month.

105. The use of embodiment 104, wherein a first administration of the engineering agent occurs at least about 2 weeks after a first administration of the nanoparticle comprising the nucleic acid encoding the immune checkpoint inhibitor whereby the conditioning is activating conditioning.

106. The use of any one of embodiments 101-105, wherein the immune checkpoint inhibitor comprises an anti-CTLA-4 antibody.

107. The use of any one of embodiments 101 to 104, wherein the immune checkpoint inhibitor comprises an anti-PD-1, anti-PD-L1, anti-Tim-3, or anti-LAG-3 antibody, wherein the conditioning is adjuvant conditioning.

108. The use of any one of embodiments 101 to 104 or 107, wherein conditioning reduces Treg cell activity.

109. The use of any one of embodiments 101 to 104 or 107, wherein conditioning activates T effector cells.

110. The use of any one of embodiments 101 to 104 or 107, wherein conditioning mobilizes immune cells into a tumor or other locus of disease.

111. The use of embodiment 91 wherein the conditioning agent comprises an inflammatory chemokine.

112. The use of embodiment 111, wherein the nanoparticle comprising the nucleic acid encoding the inflammatory chemokine is administered by intravenous, intralesional, or intraperitoneal infusion or injection.

113. The use of embodiment 111 or 112 wherein the nanoparticle comprising the nucleic acid encoding the inflammatory chemokine is administered at 3- to 4-day intervals.

114. The use of embodiment 113, wherein the nanoparticle comprising the nucleic acid encoding the inflammatory chemokine is administered 2, 3, or 4 times prior to administration of the engineering agent whereby the conditioning is activating conditioning.

115. The use of embodiment 113 or 114, wherein the engineering agent is administered the day following the most recent administration of the nanoparticle comprising the nucleic acid encoding the inflammatory chemokine.

116. The use of embodiment 113, wherein the nanoparticle comprising the nucleic acid encoding the inflammatory chemokine is administered following every 1, 2, or 3 administrations of the in vivo engineering agent, whereby the conditioning is adjuvant conditioning.

117. The use of embodiment 116, wherein the conditioning expands and/or mobilizes immune cells to a tumor or other locus of disease.

118. The use of any one of embodiments 111 to 116, wherein the inflammatory chemokine comprises CCL2, CCL3, CCL4, CCL5, CCL11, CXCL1, CXCL2, CXCL-8, CXCL9, CXCL10, or CXCL11.

119. The use of embodiment 117, wherein the inflammatory chemokine comprises CCL5.

120. The use of embodiment 91 wherein the conditioning agent comprises an agent that enhances activity of antigen presenting cells.

121. The use of embodiment 120, wherein the administration of the nanoparticle comprising the nucleic acid encoding the agent that enhances the activity of antigen presenting cells, is by intravenous, intralesional, or intraperitoneal infusion or injection.

122. The use of embodiment 120 or 121, wherein the agent that enhances the activity of antigen presenting cells is provided 3-4 days and 12-24 hours prior to the engineering agent, whereby the conditioning is adjuvant conditioning.

123. The use of any one of embodiments 120 to 122, wherein the agent that enhances the activity of antigen presenting cells is provided anytime the same day as or 12-24 hours in advance of each of multiple administrations of the engineering agent, whereby the conditioning is adjuvant conditioning.

124. The use of any one of embodiments 120-123, wherein the agent that enhances the activity of antigen presenting cells is provided every 3-7 days subsequent to a pause in or conclusion of treatment with the engineering agent while the tumor is shrinking.

125. The use of any one of embodiments 120-124, wherein epitope spreading is promoted.

126. The use of any one of embodiments 120-125, wherein the agent that enhances the activity of antigen presenting cells comprises Flt-3 ligand, gm-CSF, or IL-18.

127. The use of embodiment 91 wherein the conditioning agent comprises a pan-activating cytokine.

128. The use of embodiment 127, wherein the nanoparticle comprising the nucleic acid encoding the pan-activating cytokine is administered prior or subsequent to the engineering agent.

129. The use of embodiment 127 or 128, wherein the nanoparticle comprising the nucleic acid encoding the pan-activating cytokine is administered by intravenous, intralesional, or intraperitoneal infusion or injection.

130. The use of any one of embodiments 127 to 129, wherein the nanoparticle comprising the nucleic acid encoding the pan-activating cytokine is administered at 3- to 4-day intervals.

131. The use of any one of embodiments 127 to 130, wherein the nanoparticle comprising the nucleic acid encoding the pan-activating cytokine is administered 1, 2, 3, or 4 times prior to administration of the engineering agent which is administered 1 to 7 days after the most recent administration of the nucleic acid encoding the pan-activating cytokine, whereby the conditioning is activating conditioning.

132. The use of any one of embodiments 127 to 130, wherein the nanoparticle comprising the nucleic acid encoding the pan-activating cytokine is administered within 4 days following the most recent administration of the engineering agent, whereby the conditioning is adjuvant conditioning.

133. The use of embodiment 132, wherein the conditioning activates immune cells in a tumor or other locus of disease.

134. The use of any one of embodiments 127 to 133, wherein the pan-activating cytokine comprises IL-12 of IL 18.

135. The use of any one of embodiments 91 to 96, 99 to 106, 111 to 115, 118 to 123, 126 to 131, or 134, wherein polyfunctional effector cells are expanded.

136. The use of any one of embodiments 91 to 135, wherein the nanoparticle comprising the nucleic acid encoding the conditioning agent is a targeted nanoparticle.

137. The use of embodiment 136, wherein the targeted nanoparticle comprises a binding moiety on its surface.

138. The use of embodiment 137, wherein the binding moiety comprises an antibody antigen binding domain.

139. The use of embodiment 137 or 138, wherein the binding moiety binds to a tumor surface antigen.

140. The use of any one of embodiments 136-139, wherein the nanoparticle is a lipid nanoparticle.

141. The use of any one of embodiments 91 to 135, wherein the nanoparticle comprising the nucleic acid encoding the conditioning agent is a tropic nanoparticle.

142. The use of any one of embodiments 91 to 141, wherein the nucleic acid encoding the conditioning agent is an mRNA.

143. A use of a conditioning agent conditioning a subject who receives an engineering agent comprising providing low dose cyclophosphamide to the subject prior to administration of the engineering agent, whereby the low dose cyclophosphamide acts as an adjuvant conditioning agent.

144. The use of embodiment 143, wherein the cyclophosphamide is administered with metronomic dosing.

145. The use of embodiment 143 or 144 wherein the cyclophosphamide is administered at a dose of 50 mg daily or 100 mg every other day.

146. The use of any one of embodiments 143 to 145, wherein the cyclophosphamide is administered over a period of 5 to 8 days.

147. The use of any one of embodiments 143 to 146 wherein the cyclophosphamide is administered at a daily dose of 10-50 mg for up to 3 days.

148. The use of any one of embodiments 143 to 147, wherein the engineering agent is administered 3 to 4 days after a last dose of the cyclophosphamide.

149. The use of any one of embodiments 143 to 148, whereby Treg cell activity is reduced.

150. A use of a conditioning agent for treating a subject comprising the use of any one of embodiments 91-149, further comprising administering the engineering agent.

151. The use of any one of embodiments 91-150, wherein the engineering agent comprises a nucleic acid encoding a reprogramming agent packaged in a nanoparticle, wherein the reprogramming agent is a chimeric antigen receptor (CAR), an T cell receptor (TCR), or a T cell engager.

152. The use of embodiment 151, wherein the nucleic acid encoding the reprogramming agent is an mRNA.

153. The use of embodiment 151 or 152, wherein the nucleic acid encoding the reprogramming agent is packaged is targeted nanoparticle (tNP).

154. The use of embodiment 153, wherein the targeted nanoparticle comprises a binding moiety on its surface.

155. The use of embodiment 154, wherein the binding moiety comprises an antibody antigen binding domain.

156. The use of embodiment 154 or 155, wherein the binding moiety binds to a T cell of NK cell surface antigen.

157. The use of embodiment 156, wherein the binding moiety binds CD5.

158. The use of embodiment 156, wherein the binding moiety binds CD8.

159. The use of embodiment 156, wherein the binding moiety binds CD2.

160. The use of any one of embodiments 153-159, wherein the targeted nanoparticle is a targeted lipid nanoparticle.

161. A conditioning agent for use in conditioning a subject who receives an engineering agent, wherein conditioning the subject comprises providing a nanoparticle comprising a nucleic acid encoding the conditioning agent to the subject prior to, concurrently with, or subsequent to administration of the engineering agent, wherein the conditioning agent comprises a γ-chain receptor agonist, an inflammatory chemokine, a pan-activating cytokine, an antigen presenting cell activity enhancer, a CTLA-4 checkpoint inhibitor, an immune checkpoint inhibitor or an anti-CCR4 antibody.

162. The conditioning agent of embodiment 161, wherein the conditioning agent comprises a γ-chain receptor agonist.

163. The conditioning agent of embodiment 162, wherein the nanoparticle is administered by intravenous or subcutaneous infusion or injection.

164. The conditioning agent of embodiment 162 or 163, wherein the nanoparticle is provided to the subject by 3 weekly administrations.

165. The conditioning agent of embodiment 164, wherein the third administration is 3 to 7 days before the subject receives the engineering agent, whereby the conditioning is activating conditioning.

166. The conditioning agent of any one of embodiments 161 to 165, wherein conditioning increases the number of polyfunctional immune effector cells.

167. The conditioning agent of embodiment 164, wherein at least one of the weekly administrations occurs after the subject receives the engineering agent, whereby the conditioning is adjuvant conditioning.

168. The conditioning agent of any one of embodiments 161-164, or 167, wherein conditioning leads to mobilization of reprogrammed cells into a tumor or other locus of disease subsequent to administration of the engineering agent.

169. The conditioning agent of embodiment of any one of embodiments 161 to 168, wherein the γ-chain receptor agonists comprises a γ-chain receptor cytokine.

170. The conditioning agent of embodiment 169, wherein the γ-chain receptor cytokine comprises IL-15, IL-2, IL-7, or IL-21.

171. The conditioning agent of embodiment 161, wherein the conditioning agent comprises an immune checkpoint inhibitor.

172. The conditioning agent of embodiment 171, comprising providing the nanoparticle comprising the nucleic acid encoding the immune checkpoint inhibitor to the subject prior to, concurrently with, or subsequent to administration of the engineering agent.

173. The conditioning agent of embodiment 171 or 172, wherein the nanoparticle is administered by intravenous or subcutaneous infusion or injection.

174. The conditioning agent of any one of embodiments 171 to 173, wherein administration of the nanoparticle comprising the nucleic acid encoding the immune checkpoint inhibitor occurs every 3 to 7 days over a period of 1 week to 1 month.

175. The conditioning agent of embodiment 174, wherein a first administration of the engineering agent occurs at least about 2 weeks after a first administration of the nanoparticle comprising the nucleic acid encoding the immune checkpoint inhibitor whereby the conditioning is activating conditioning.

176. The conditioning agent of any one of embodiments 171-175, wherein the immune checkpoint inhibitor comprises an anti-CTLA-4 antibody.

177. The conditioning agent of any one of embodiments 171 to 174, wherein the immune checkpoint inhibitor comprises an anti-PD-1, anti-PD-L1, anti-Tim-3, or anti-LAG-3 antibody, wherein the conditioning is adjuvant conditioning.

178. The conditioning agent of any one of embodiments 171 to 174 or 177, wherein conditioning reduces Treg cell activity.

179. The conditioning agent of any one of embodiments 171 to 174 or 177, wherein conditioning activates T effector cells.

180. The conditioning agent of any one of embodiments 171 to 174 or 177, wherein conditioning mobilizes immune cells into a tumor or other locus of disease.

181. The conditioning agent of embodiment 161 wherein the conditioning agent comprises an inflammatory chemokine.

182. The conditioning agent of embodiment 181, wherein the nanoparticle comprising the nucleic acid encoding the inflammatory chemokine is administered by intravenous, intralesional, or intraperitoneal infusion or injection.

183. The conditioning agent of embodiment 181 or 182 wherein the nanoparticle comprising the nucleic acid encoding the inflammatory chemokine is administered at 3- to 4-day intervals.

184. The conditioning agent of embodiment 183, wherein the nanoparticle comprising the nucleic acid encoding the inflammatory chemokine is administered 2, 3, or 4 times prior to administration of the engineering agent whereby the conditioning is activating conditioning.

185. The conditioning agent of embodiment 183 or 184, wherein the engineering agent is administered the day following the most recent administration of the nanoparticle comprising the nucleic acid encoding the inflammatory chemokine.

186. The conditioning agent of embodiment 183, wherein the nanoparticle comprising the nucleic acid encoding the inflammatory chemokine is administered following every 1, 2, or 3 administrations of the in vivo engineering agent, whereby the conditioning is adjuvant conditioning.

187. The conditioning agent of embodiment 186, wherein the conditioning expands and/or mobilizes immune cells to a tumor or other locus of disease.

188. The conditioning agent of any one of embodiments 181 to 186, wherein the inflammatory chemokine comprises CCL2, CCL3, CCL4, CCL5, CCL11, CXCL1, CXCL2, CXCL-8, CXCL9, CXCL10, or CXCL11.

189. The conditioning agent of embodiment 187, wherein the inflammatory chemokine comprises CCL5.

190. The conditioning agent of embodiment 161 wherein the conditioning agent comprises an agent that enhances activity of antigen presenting cells.

191. The conditioning agent of embodiment 190, wherein the administration of the nanoparticle comprising the nucleic acid encoding the agent that enhances the activity of antigen presenting cells, is by intravenous, intralesional, or intraperitoneal infusion or injection.

192. The conditioning agent of embodiment 190 or 191, wherein the agent that enhances the activity of antigen presenting cells is provided 3-4 days and 12-24 hours prior to the engineering agent, whereby the conditioning is adjuvant conditioning.

193. The conditioning agent of any one of embodiments 190 to 192, wherein the agent that enhances the activity of antigen presenting cells is provided anytime the same day as or 12-24 hours in advance of each of multiple administrations of the engineering agent, whereby the conditioning is adjuvant conditioning.

194. The conditioning agent of any one of embodiments 190-193, wherein the agent that enhances the activity of antigen presenting cells is provided every 3-7 days subsequent to a pause in or conclusion of treatment with the engineering agent while the tumor is shrinking.

195. The conditioning agent of any one of embodiments 190-194, wherein epitope spreading is promoted.

196. The conditioning agent of any one of embodiments 190-195, wherein the agent that enhances the activity of antigen presenting cells comprises Flt-3 ligand, gm-CSF, or IL-18.

197. The conditioning agent of embodiment 161 wherein the conditioning agent comprises a pan-activating cytokine.

198. The conditioning agent of embodiment 197, wherein the nanoparticle comprising the nucleic acid encoding the pan-activating cytokine is administered prior or subsequent to the engineering agent.

199. The conditioning agent of embodiment 197 or 198, wherein the nanoparticle comprising the nucleic acid encoding the pan-activating cytokine is administered by intravenous, intralesional, or intraperitoneal infusion or injection.

200. The conditioning agent of any one of embodiments 197 to 199, wherein the nanoparticle comprising the nucleic acid encoding the pan-activating cytokine is administered at 3- to 4-day intervals.

201. The conditioning agent of any one of embodiments 197 to 200, wherein the nanoparticle comprising the nucleic acid encoding the pan-activating cytokine is administered 1, 2, 3, or 4 times prior to administration of the engineering agent which is administered 1 to 7 days after the most recent administration of the nucleic acid encoding the pan-activating cytokine, whereby the conditioning is activating conditioning.

202. The conditioning agent of any one of embodiments 197 to 200, wherein the nanoparticle comprising the nucleic acid encoding the pan-activating cytokine is administered within 4 days following the most recent administration of the engineering agent, whereby the conditioning is adjuvant conditioning.

203. The conditioning agent of embodiment 202, wherein the conditioning activates immune cells in a tumor or other locus of disease.

204. The conditioning agent of any one of embodiments 197 to 203, wherein the pan-activating cytokine comprises IL-12 of IL 18.

205. The conditioning agent of any one of embodiments 161 to 166, 169 to 176, 181 to 185, 188 to 193, 196 to 201, or 204, wherein polyfunctional effector cells are expanded.

206. The conditioning agent of any one of embodiments 161 to 205, wherein the nanoparticle comprising the nucleic acid encoding the conditioning agent is a targeted nanoparticle.

207. The conditioning agent of embodiment 206, wherein the targeted nanoparticle comprises a binding moiety on its surface.

208. The conditioning agent of embodiment 207, wherein the binding moiety comprises an antibody antigen binding domain.

209. The conditioning agent of embodiment 207 or 208, wherein the binding moiety binds to a tumor surface antigen.

210. The conditioning agent of any one of embodiments 206-209, wherein the nanoparticle is a lipid nanoparticle.

211. The conditioning agent of any one of embodiments 161 to 205, wherein the nanoparticle comprising the nucleic acid encoding the conditioning agent is a tropic nanoparticle.

212. The conditioning agent of any one of embodiments 161 to 211, wherein the nucleic acid encoding the conditioning agent is an mRNA.

213. A conditioning agent for use in conditioning a subject who receives an engineering agent comprising providing a conditioning agent to the subject prior to administration of the engineering agent, whereby the low dose cyclophosphamide acts as the conditioning agent.

214. The conditioning agent of embodiment 213, wherein the cyclophosphamide is administered with metronomic dosing.

215. The conditioning agent of embodiment 213 or 214 wherein the cyclophosphamide is administered at a dose of 50 mg daily or 100 mg every other day.

216. The conditioning agent of any one of embodiments 213 to 215, wherein the cyclophosphamide is administered over a period of 5 to 8 days.

217. The conditioning agent of any one of embodiments 213 to 216 wherein the cyclophosphamide is administered at a daily dose of 10-50 mg for up to 3 days.

218. The conditioning agent of any one of embodiments 213 to 217, wherein the engineering agent is administered 3 to 4 days after a last dose of the cyclophosphamide.

219. The conditioning agent of any one of embodiments 213 to 218, whereby Treg cell activity is reduced.

220. A conditioning agent for use in treating a subject comprising administering the conditioning agent of any one of embodiments 161-219, further comprising administering the engineering agent.

221. The conditioning agent of any one of embodiments 161-220, wherein the engineering agent comprises a nucleic acid encoding a reprogramming agent packaged in a nanoparticle, wherein the reprogramming agent is a chimeric antigen receptor (CAR), an T cell receptor (TCR), or a T cell engager.

222. The conditioning agent of embodiment 221, wherein the nucleic acid encoding the reprogramming agent is an mRNA.

223. The conditioning agent of embodiment 221 or 222, wherein the nucleic acid encoding the reprogramming agent is packaged is targeted nanoparticle (tNP).

224. The conditioning agent of embodiment 223, wherein the targeted nanoparticle comprises a binding moiety on its surface.

225. The conditioning agent of embodiment 224, wherein the binding moiety comprises an antibody antigen binding domain.

226. The conditioning agent of embodiment 224 or 225, wherein the binding moiety binds to a T cell of NK cell surface antigen.

227. The conditioning agent of embodiment 226, wherein the binding moiety binds CD5.

228. The conditioning agent of embodiment 226, wherein the binding moiety binds CD8.

229. The conditioning agent of embodiment 226, wherein the binding moiety binds CD2.

230. The conditioning agent of any one of embodiments 223-229, wherein the targeted nanoparticle is a targeted lipid nanoparticle.

1. A method of conditioning a subject who receives an engineering agent comprising providing a nanoparticle comprising a nucleic acid encoding a conditioning agent to the subject prior to, concurrently with, or subsequent to administration of the engineering agent, wherein the conditioning agent comprises a γ-chain receptor agonist, an inflammatory chemokine, a pan-activating cytokine, an antigen presenting cell activity enhancer, a CTLA-4 checkpoint inhibitor, an immune checkpoint inhibitor or an anti-CCR4 antibody.

2. The method of claim 1, wherein the conditioning agent comprises a γ-chain receptor agonist.

3. The method of claim 2, wherein the nanoparticle is administered by intravenous or subcutaneous infusion or injection.

4. The method of claim 2 or 3, wherein the nanoparticle is provided to the subject by 3 weekly administrations.

5. The method of claim 4, wherein the third administration is 3 to 7 days before the subject receives the engineering agent, whereby the conditioning is activating conditioning.

6. The method of any one of claims 1 to 5, wherein conditioning increases the number of polyfunctional immune effector cells.

7. The method of claim 4, wherein at least one of the weekly administrations occurs after the subject receives the engineering agent, whereby the conditioning is adjuvant conditioning.

8. The method of any one of claims 1-4, or 7, wherein conditioning leads to mobilization of reprogrammed cells into a tumor or other locus of disease subsequent to administration of the engineering agent.

9. The method of claim of any one of claims 1 to 8, wherein the γ-chain receptor agonists comprises a γ-chain receptor cytokine.

10. The method of claim 9, wherein the γ-chain receptor cytokine comprises IL-15, IL-2, IL-7, or IL-21.

11. The method of claim 1, wherein the conditioning agent comprises an immune checkpoint inhibitor.

12. The method of claim 11, comprising providing the nanoparticle comprising the nucleic acid encoding the immune checkpoint inhibitor to the subject prior to, concurrently with, or subsequent to administration of the engineering agent.

13. The method of claim 11 or 12, wherein the nanoparticle is administered by intravenous or subcutaneous infusion or injection.

14. The method of any one of claims 11 to 13, wherein administration of the nanoparticle comprising the nucleic acid encoding the immune checkpoint inhibitor occurs every 3 to 7 days over a period of 1 week to 1 month.

15. The method of claim 14, wherein a first administration of the engineering agent occurs at least about 2 weeks after a first administration of the nanoparticle comprising the nucleic acid encoding the immune checkpoint inhibitor whereby the conditioning is activating conditioning.

16. The method of any one of claims 11-15, wherein the immune checkpoint inhibitor comprises an anti-CTLA-4 antibody.

17. The method of any one of claims 11 to 14, wherein the immune checkpoint inhibitor comprises an anti-PD-1, anti-PD-L1, anti-Tim-3, or anti-LAG-3 antibody, wherein the conditioning is adjuvant conditioning.

18. The method of any one of claims 11 to 14 or 17, wherein conditioning reduces Treg cell activity.

19. The method of any one of claims 11 to 14 or 17, wherein conditioning activates T effector cells.

20. The method of any one of claims 11 to 14 or 17, wherein conditioning mobilizes immune cells into a tumor or other locus of disease.

21. The method of claim 1 wherein the conditioning agent comprises an inflammatory chemokine.

22. The method of claim 21, wherein the nanoparticle comprising the nucleic acid encoding the inflammatory chemokine is administered by intravenous, intralesional, or intraperitoneal infusion or injection.

23. The method of claim 21 or 22 wherein the nanoparticle comprising the nucleic acid encoding the inflammatory chemokine is administered at 3- to 4-day intervals.

24. The method of claim 23, wherein the nanoparticle comprising the nucleic acid encoding the inflammatory chemokine is administered 2, 3, or 4 times prior to administration of the engineering agent whereby the conditioning is activating conditioning.

25. The method of claim 23 or 24, wherein the engineering agent is administered the day following the most recent administration of the nanoparticle comprising the nucleic acid encoding the inflammatory chemokine.

26. The method of claim 23, wherein the nanoparticle comprising the nucleic acid encoding the inflammatory chemokine is administered following every 1, 2, or 3 administrations of the in vivo engineering agent, whereby the conditioning is adjuvant conditioning.

27. The method of claim 26, wherein the conditioning expands and/or mobilizes immune cells to a tumor or other locus of disease.

28. The method of any one of claims 21 to 26, wherein the inflammatory chemokine comprises CCL2, CCL3, CCL4, CCL5, CCL1, CXCL1, CXCL2, CXCL-8, CXCL9, CXCL10, or CXCL11.

29. The method of claim 27, wherein the inflammatory chemokine comprises CCL5.

30. The method of claim 1 wherein the conditioning agent comprises an agent that enhances activity of antigen presenting cells.

31. The method of claim 30, wherein the administration of the nanoparticle comprising the nucleic acid encoding the agent that enhances the activity of antigen presenting cells, is by intravenous, intralesional, or intraperitoneal infusion or injection.

32. The method of claim 30 or 31, wherein the agent that enhances the activity of antigen presenting cells is provided 3-4 days and 12-24 hours prior to the engineering agent, whereby the conditioning is adjuvant conditioning.

33. The method of any one of claims 30 to 32, wherein the agent that enhances the activity of antigen presenting cells is provided anytime the same day as or 12-24 hours in advance of each of multiple administrations of the engineering agent, whereby the conditioning is adjuvant conditioning.

34. The method of any one of claims 30-33, wherein the agent that enhances the activity of antigen presenting cells is provided every 3-7 days subsequent to a pause in or conclusion of treatment with the engineering agent while the tumor is shrinking.

35. The method of any one of claims 30-34, wherein epitope spreading is promoted.

36. The method of any one of claims 30-35, wherein the agent that enhances the activity of antigen presenting cells comprises Flt-3 ligand, gm-CSF, or IL-18.

37. The method of claim 1 wherein the conditioning agent comprises a pan-activating cytokine.

38. The method of claim 37, wherein the nanoparticle comprising the nucleic acid encoding the pan-activating cytokine is administered prior or subsequent to the engineering agent.

39. The method of claim 37 or 38, wherein the nanoparticle comprising the nucleic acid encoding the pan-activating cytokine is administered by intravenous, intralesional, or intraperitoneal infusion or injection.

40. The method of any one of claims 37 to 39, wherein the nanoparticle comprising the nucleic acid encoding the pan-activating cytokine is administered at 3- to 4-day intervals.

41. The method of any one of claims 37 to 40, wherein the nanoparticle comprising the nucleic acid encoding the pan-activating cytokine is administered 1, 2, 3, or 4 times prior to administration of the engineering agent which is administered 1 to 7 days after the most recent administration of the nucleic acid encoding the pan-activating cytokine, whereby the conditioning is activating conditioning.

42. The method of any one of claims 37 to 40, wherein the nanoparticle comprising the nucleic acid encoding the pan-activating cytokine is administered within 4 days following the most recent administration of the engineering agent, whereby the conditioning is adjuvant conditioning.

43. The method of claim 42, wherein the conditioning activates immune cells in a tumor or other locus of disease.

44. The method of any one of claims 37 to 43, wherein the pan-activating cytokine comprises IL-12 of IL 18.

45. The method of any one of claims 1 to 6, 9 to 16, 21 to 25, 28 to 33, 36 to 41, or 44, wherein polyfunctional effector cells are expanded.

46. The method of any one of claims 1 to 45, wherein the nanoparticle comprising the nucleic acid encoding the conditioning agent is a targeted nanoparticle.

47. The method of claim 46, wherein the targeted nanoparticle comprises a binding moiety on its surface.

48. The method of claim 47, wherein the binding moiety comprises an antibody antigen binding domain.

49. The method of claim 47 or 48, wherein the binding moiety binds to a tumor surface antigen.

50. The method of any one of embodiments 46-49, wherein the nanoparticle is a lipid nanoparticle.

51. The method of any one of claims 1 to 45, wherein the nanoparticle comprising the nucleic acid encoding the conditioning agent is a tropic nanoparticle.

52. The method of any one of claims 1 to 51, wherein the nucleic acid encoding the conditioning agent is an mRNA.

53. A method of conditioning a subject who receives an engineering agent comprising providing low dose cyclophosphamide to the subject prior to administration of the engineering agent, whereby the low dose cyclophosphamide acts as an adjuvant conditioning agent.

54. The method of claim 53, wherein the cyclophosphamide is administered with metronomic dosing.

55. The method of claim 53 or 54 wherein the cyclophosphamide is administered at a dose of 50 mg daily or 100 mg every other day.

56. The method of any one of claims 53 to 55, wherein the cyclophosphamide is administered over a period of 5 to 8 days.

57. The method of any one of claims 53 to 56 wherein the cyclophosphamide is administered at a daily dose of 10-50 mg for up to 3 days.

58. The method of any one of claims 53 to 57, wherein the engineering agent is administered 3 to 4 days after a last dose of the cyclophosphamide.

59. The method of any one of claims 53 to 58, whereby Treg cell activity is reduced.

60. A method of treatment comprising the method of any one of claims 1-59, further comprising administering the engineering agent.

61. The method of any one of claims 1-60, wherein the engineering agent comprises a nucleic acid encoding a reprogramming agent packaged in a nanoparticle, wherein the reprogramming agent is a chimeric antigen receptor (CAR), an T cell receptor (TCR), or a T cell engager.

62. The method of claim 61, wherein the nucleic acid encoding the reprogramming agent is an mRNA.

63. The method of claim 62 or 62, wherein the nucleic acid encoding the reprogramming agent is packaged is targeted nanoparticle (tNP).

64. The method of claim 63, wherein the targeted nanoparticle comprises a binding moiety on its surface.

65. The method of claim 64, wherein the binding moiety comprises an antibody antigen binding domain.

66. The method of claim 64 or 65, wherein the binding moiety binds to a T cell of NK cell surface antigen.

67. The method of claim 66, wherein the binding moiety binds CD5.

68. The method of claim 66, wherein the binding moiety binds CD8.

69. The method of claim 66, wherein the binding moiety binds CD2.

70. The method of any one of claims 63-69, wherein the targeted nanoparticle is a targeted lipid nanoparticle.
